(19) United States
(12) Patent Application Publication
(10) Pub. No.: US 2025/0297007 A1
(43) Pub. Date: Sep. 25, 2025

(54) MULTIFUNCTIONAL MOLECULES BINDING TO TCR AND USES THEREOF

(71) Applicant: Marengo Therapeutics, Inc., Cambridge, MA (US)

(72) Inventors: Andrew Bayliffe, London (GB); Jessica Lynn Lowry, Southborough, MA (US); Jacques Moisan, Acton, MA (US); Madan Katragadda, Acton, MA (US); Gurkan Guntas, Newton, MA (US)

(21) Appl. No.: 19/175,269

(22) Filed: Apr. 10, 2025

Related U.S. Application Data
(63) Continuation of application No. PCT/US23/35056, filed on Oct. 12, 2023.
(60) Provisional application No. 63/379,243, filed on Oct. 12, 2022.

(51) Int. Cl. C07K 16/28; A61K 39/00; A61P 35/00; C07K 14/55; C07K 16/30

(52) U.S. Cl. CPC C07K 16/2809; A61P 35/00; C07K 14/55; C07K 16/2887; C07K 16/30; A61K 2039/505; C07K 2317/24; C07K 2317/31; C07K 2317/33; C07K 2317/622; C07K 2317/77; C07K 2317/92; C07K 2317/94

(57) ABSTRACT

Provides herein are multifunctional molecules comprising T cell receptor variable beta-binding moieties and cytokines, and methods of treating conditions or diseases in a subject using the same.

### CROSS REFERENCE

This application is a continuation of International Application No. PCT/US2023/035056 filed Oct. 12, 2023, which claims the benefit of U.S. Provisional Patent Application No. 63/379,243 filed Oct. 12, 2022, each of which is incorporated herein by reference in its entirety.

### SEQUENCE LISTING

The instant application contains a Sequence listing which has been submitted electronically in XML format and is herein incorporated by reference in its entirety. Said XML copy, created on Dec. 15, 2023, is named 53676-756_601_SL.xml and is 1,750,290 bytes in size.

### BACKGROUND

Currently available molecules designed to redirect T cells to promote tumor cell lysis for cancer immunotherapy typically target the CD3 epsilon (CD3e) subunit of the T cell receptor (TCR). However, there are limitations to this approach. Previous studies have shown that, e.g., low doses of anti-CD3e monoclonal antibody (mAb) can cause T cell dysfunction and exert immunosuppressive effects. In addition, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Thus, there is a need for improved T cell receptor-binding molecules that redirect T cells for cancer immunotherapy.

### SUMMARY

In an aspect, provided herein is, inter alia, a multifunctional molecule comprising: (a) a tumor-associated antigen binding moiety; (b) at least one cytokine molecule or a functional fragment or functional variant thereof, and (c) a TCRβV-binding moiety covalently linked to the at least one cytokine molecule or a functional fragment or functional variant thereof.

In some embodiments, the multifunctional molecule comprises a first polypeptide chain comprising a first portion of a dimerization module, and a second polypeptide chain comprising a second portion of the dimerization module; wherein the first polypeptide chain and the second polypeptide chain are non-contiguous, and wherein the tumor-associated antigen binding moiety is linked to the first portion of the dimerization module, and the at least one cytokine molecule or a functional fragment or functional variant thereof is linked to the first portion of the dimerization module, the second portion of the dimerization module, or a combination thereof.

In some embodiments, (i) the tumor-associated antigen binding moiety is linked to the N-terminus of the first portion of the dimerization module, and the at least one cytokine molecule or a functional fragment or functional variant thereof is linked to the C-terminus of the first portion of the dimerization module, the N-terminus of the second portion of the dimerization module, the C-terminus of the second portion of the dimerization module, or any combination thereof; or (ii) the tumor-associated antigen binding moiety is linked to the C-terminus of the first portion of the dimerization module, and the at least one cytokine molecule or a functional fragment or functional variant thereof is linked to the N-terminus of the first portion of the dimerization module, the N-terminus of the second portion of the dimerization module, the C-terminus of the second portion of the dimerization module, or any combination thereof.

In some embodiments, the TCRβV-binding moiety and the at least one cytokine molecule or a functional fragment or functional variant thereof is within a single contiguous polypeptide chain of the first polypeptide chain or the second polypeptide chain.

In some embodiments, the tumor-associated antigen binding moiety, the TCRβV-binding moiety, or a combination thereof comprises an antibody or antigen binding fragment thereof, wherein the antigen binding fragment comprises any one selected from the group consisting of a Fab, a F(ab′)2, an Fv, a single chain Fv (scFv), a single domain antibody, a diabody (dAb), a camelid antibody, and any combination thereof.

In some embodiments, the TCRβV-binding moiety comprises a heavy chain variable domain (VH) and a light chain variable domain (VL), or a single domain antibody.

In some embodiments, the TCRβV-binding moiety comprises a first portion of the TCRβV-binding moiety, and wherein the multifunctional molecule further comprises a third polypeptide chain comprising a second portion of the TCRβV-binding moiety, wherein the third polypeptide chain is non-contiguous with the first polypeptide chain and the second polypeptide chain.

In some embodiments, the first portion of the TCRβV-binding moiety comprises a VH of the TCRβV-binding moiety and the second portion of the TCRβV-binding moiety comprises a VL of the TCRβV-binding moiety, or the first portion of the TCRβV-binding moiety comprises a VL of the TCRβV-binding moiety and the second portion of the TCRβV-binding moiety comprises a VH of the TCRβV-binding moiety.

In some embodiments, the tumor-associated antigen binding moiety comprises a VH and a VL, or a single domain antibody.

In some embodiments, the tumor-associated antigen binding moiety comprises a first portion of the tumor-associated antigen binding moiety, and wherein the multifunctional molecule further comprises a fourth polypeptide chain comprising a second portion of the tumor-associated antigen binding moiety, wherein the fourth polypeptide chain is non-contiguous with the first polypeptide chain, the second polypeptide chain, and the third polypeptide chain.

In some embodiments, the first portion of the tumor-associated antigen binding moiety comprises a VH of the tumor-associated antigen binding moiety and the second portion of the tumor-associated antigen binding moiety comprises a VL of the tumor-associated antigen binding moiety, or the first portion of the tumor-associated antigen binding moiety comprises a VL of the tumor-associated antigen binding moiety and the second portion of the tumor-associated antigen binding moiety comprises a VH of the tumor-associated antigen binding moiety.

In some embodiments, the first portion of the dimerization module and the second portion of the dimerization module are dimerized.

In some embodiments. (i) the tumor-associated antigen binding moiety further comprises a heavy chain constant domain 1 (CH1) linked to the VH of the tumor-associated antigen binding moiety; (ii) the TCRβV-binding moiety further comprises a heavy chain constant domain 1 (CH1) linked to the VH of the TCRβV-binding moiety; or (iii) a combination thereof.

In some embodiments, (i) the tumor-associated antigen binding moiety further comprises a light chain constant domain (CL) linked to the VL of the tumor-associated antigen binding moiety; (ii) the TCRβV-binding moiety further comprises a light chain constant domain (CL) linked to the VL of the TCRβV-binding moiety; or (iii) a combination thereof.

In some embodiments. (i) the CL linked to the VL of the tumor-associated antigen binding moiety comprises a kappa chain constant domain or a lambda chain constant domain; (ii) the CL linked to the VL of the TCRβV-binding moiety comprises a kappa chain constant domain or a lambda chain constant domain; or (iii) a combination thereof.

In some embodiments, the kappa chain constant domain or the lambda chain constant domain comprises any one of the light chain constant region sequences listed in Table 3, 21, or 22.

In some embodiments, the multifunctional molecule as provided herein further comprises: (i) a linker between the first portion of the dimerization module and the tumor-associated antigen binding moiety or the first portion of the tumor-associated antigen; (ii) a linker between the at least one cytokine molecule or a functional fragment or functional variant thereof and the first portion of the dimerization module, a linker between the at least one cytokine molecule or a functional fragment or functional variant thereof and the second portion of the dimerization module, or any combination thereof; (iii) a linker between the at least one cytokine molecule or a functional fragment or functional variant thereof and the TCRβV-binding moiety or the first portion of the TCRβV-binding moiety; (iv) a linker between the VH and the VL of the tumor-associated antigen binding moiety; (v) a linker between the VH and the VL of the TCRβV-binding moiety; (vi) a linker between the CH1 and the VH of the tumor-associated antigen binding moiety; (vii) a linker between the CH1 and the VH of the TCRβV-binding moiety; (viii) a linker between the CL and the VL of the tumor-associated antigen binding moiety; (ix) a linker between the CL and the VL of the TCRβV-binding moiety; or (x) any combination thereof.

In some embodiments, the linker is selected from the group consisting of a cleavable linker, a non-cleavable linker, a peptide linker, a flexible linker, a rigid linker, a helical linker, and a non-helical linker.

In some embodiments, the linker is the peptide linker, and wherein the linker comprises the sequence of SEQ ID NO: 3308 or SEQ ID NO: 3643.

In some embodiments, the multifunctional molecule is an isolated multifunctional molecule.

In some embodiments, the tumor-associated antigen binding moiety, the TCRβV-binding moiety, or a combination thereof comprises the Fab or the scFv.

In some embodiments, the at least one cytokine molecule or a functional fragment or functional variant thereof is selected from the group consisting of interleukin-2 (IL-2) or a functional fragment or functional variant thereof, interleukin-7 (IL-7) or a functional fragment or functional variant thereof, interleukin-12 (IL-12) or a functional fragment or functional variant thereof, interleukin-15 (IL-15) or a functional fragment or functional variant thereof, interleukin-18 (IL-18) or a functional fragment or functional variant thereof, interleukin-21 (IL-21) or a functional fragment or functional variant thereof, or interferon gamma or a functional fragment or functional variant thereof, or any combination thereof.

In some embodiments, the at least one cytokine molecule or a functional fragment or functional variant thereof comprises interleukin-2 (IL-2) or a functional fragment or functional variant thereof.

In some embodiments, the at least one cytokine molecule or a functional fragment or functional variant thereof is an IL-2 variant comprising a substitution mutation.

In some embodiments, the at least one cytokine molecule or a functional fragment or functional variant thereof is an IL-2 variant comprising C125A mutation.

In some embodiments, the at least one cytokine molecule or a functional fragment or functional variant thereof comprises a sequence having at least 75% sequence identity to the sequence of SEQ ID NO: 2270 or the sequence of SEQ ID NO: 2191.

In some embodiments, the at least one cytokine molecule or a functional fragment or functional variant thereof comprises the sequence of SEQ ID NO: 2270 or the sequence of SEQ ID NO: 2191.

In some embodiments, the first portion of the dimerization module comprises a first immunoglobulin constant regions (Fc regions) and the second portion of the dimerization module comprises a second Fc region.

In some embodiments, the first Fc region, the second Fc region, or a combination thereof is selected from the group consisting of an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from the group consisting of a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

In some embodiments, the first Fc region, the second Fc region, or a combination thereof comprises an amino acid substitution listed in Table 14, 21, or 22.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75% sequence identity to the sequence of SEQ ID NO: 40, SEQ ID NO: 42, SEQ ID NO: 3645, SEQ ID NO: 3646, SEQ ID NO: 3647, SEQ ID NO:3648, SEQ ID NO: 3649, SEQ ID NO: 3792, or SEQ ID NO: 3794.

In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, SEQ ID NO: 3649, SEQ ID NO: 3792, or SEQ ID NO: 3794.

In some embodiments, the first Fc region comprises a sequence having at least 75% sequence identity to the sequence of SEQ ID NO: 3649 or SEQ ID NO: 3792, and the second Fc region comprises a sequence having at least 75% sequence identity to the sequence of SEQ ID NO: 3648 or SEQ ID NO: 3794, or the first Fc region comprises a sequence having at least 75% sequence identity to the sequence of SEQ ID NO: 3648 or SEQ ID NO: 3794, and the second Fc region comprises a sequence having at least 75% sequence identity to the sequence of SEQ ID NO: 3648 or SEQ ID NO: 3794.

In some embodiments, the first Fc region comprises a sequence having the sequence of SEQ ID NO: 3649 or SEQ ID NO: 3792, and the second Fc region comprises the sequence of SEQ ID NO: 3648 or SEQ ID NO: 3794, or the first Fc region comprises the sequence of SEQ ID NO: 3648 or SEQ ID NO: 3794, and the second Fc region comprises the sequence of SEQ ID NO: 3648 or SEQ ID NO: 3794.

In some embodiments, the TCRβV-binding moiety binds to one or more of a TCRβV subfamily selected from the group consisting of TCRβ V2 subfamily, TCRβ V3 subfamily, TCRβ V4 subfamily, TCRβ V5 subfamily, TCRβ V6 subfamily, TCRβ V9 subfamily, TCRβ V10 subfamily, TCRβ V11 subfamily, TCRβ V12 subfamily, TCRβ V13 subfamily, TCRβ V16 subfamily, TCRβ V19, TCRβ V20 subfamily, TCRβ V21 subfamily, TCRβ V23 subfamily, TCRβ V27 subfamily, and TCRβ V28 subfamily.

In some embodiments, the TCRβV-binding moiety binds to one or more of a TCRβV subfamily selected from the group consisting of: (i) TCRβ V2 subfamily comprising TCRβ V2*01; (ii) TCRβ V3 subfamily comprising TCRβ V3-1*01; (iii) TCRβ V4 subfamily comprising one or more selected from TCRB V4-1, TCRβ V4-2, and TCRβ V4-3; (iv) TCRβ V5 subfamily comprising one or more selected from TCRB V5-6*01, TCRβ V5-4*01, TCRβ V5-1*01, and TCRβ V5-8*01; (v) TCRβ V6 subfamily comprising one or more selected from TCRβ V6-4*01, TCRβ V6-4*02, TCRβ V6-9*01, TCRβ V6-8*01, TCRβ V6-5*01. TCRβ V6-6*02, TCRβ V6-6*01, TCRβ V6-2*01, TCRβ V6-3*01, and TCRβ V6-1*01; (vi) TCRβ V10 subfamily comprising one or more selected from TCRβ V10-1*01, TCRβ V10-1*02, TCRβ V10-3*01, and TCRβ V10-2*01; (vii) TCRβ V11 subfamily comprising TCRβ V11-2; (viii) TCRβ V12 subfamily comprising one or more selected from TCRβ V12-4*01, TCRβ V12-3*01, and TCRβ V12-5*01; (ix) TCRβ V13 subfamily comprising TCRβ V13*01; (x) TCRβ V16 subfamily comprising TCRβ V16*01; (xi) TCRβ V19 subfamily comprising one or more selected from TCRβ V19*01 and TCRβ V19*02; or (xii) TCRβ V20 subfamily comprising TCRβ V20-1*01, or TCRβ V20-1*02.

In some embodiments, the TCRβV-binding moiety binds to TCRβ V6 subfamily or TCRβ V20 subfamily.

In some embodiments, the TCRβV-binding moiety comprises: (i) a VH comprising a combination of a HC CDR1, a HC CDR2 and a HC CDR3 listed in Tables 1, 2, 10B, 11, 12, 13, 21, or 22; (ii) a VL comprising a combination of a LC CDR1, a LC CDR2, and a LC CDR3 listed in Tables 1, 2, 10B, 11, 12, 13, 21, or 22; or (iii) a combination thereof.

In some embodiments, the TCRβV-binding moiety comprises: (i) a VH comprising a HC CDR1, a HC CDR2 and a HC CDR3 of any one of the heavy chain variable domain amino acid sequences listed in Tables 1, 2, 10B, 11, 12, 13, 21, or 22; (ii) a VL comprising a LC CDR1, a LC CDR2, and a LC CDR3 of any one of the light chain variable domain amino acid sequences listed in Tables 1, 2, 10B, 11, 12, 13, 21, or 22; or (iii) a combination thereof.

In some embodiments, the TCRβV-binding moiety comprises: (i) a VH comprising a sequence having at least 70% sequence identity to any one of the heavy chain variable domain amino acid sequences listed in Tables 1, 2, 10B, 11, 12, 13, 21, or 22; (i) a VL comprising a sequence having at least 70% sequence identity to any one of the light chain variable domain amino acid sequences listed in Tables 1, 2, 10B, 11, 12, 13, 21, or 22; (iii) a combination thereof.

In some embodiments, the TCRβV-binding moiety comprises: (i) a VH comprising any one of the heavy chain variable domain amino acid sequences listed in Tables 1, 2, 10B, 11, 12, 13, 21, or 22; (ii) a VL comprising any one of the light chain variable domain amino acid sequences listed in Tables 1, 2, 10B, 11, 12, 13, 21, or 22. (iii) a combination thereof.

In some embodiments, the second polypeptide chain comprises a sequence having at least 70% sequence identity to the sequence of SEQ ID NO: 1346, a sequence having at least 70% sequence identity to the sequence of SEQ ID NO: 1349, a sequence having at least 70% sequence identity to the sequence of SEQ ID NO: 2270, and a sequence having at least 70% sequence identity to the sequence of SEQ ID NO: 3648.

In some embodiments, the second polypeptide chain comprises the sequence of SEQ ID NO: 1346, the sequence of SEQ ID NO: 1349, the sequence of SEQ ID NO: 2270, and the sequence of SEQ ID NO: 3648.

In some embodiments, the second polypeptide chain further comprises the sequence of SEQ ID NO: 3801, the sequence of SEQ ID NO: 3309, the sequence of SEQ ID NO: 3308, or any combination thereof.

In some embodiments, the second polypeptide chain comprises a sequence having at least 70% sequence identity to the sequence of SEQ ID NO: 1346 operatively linked to a sequence having at least 70% sequence identity to the sequence of SEQ ID NO: 1349 operatively linked to a sequence having at least 70% sequence identity to the sequence of SEQ ID NO: 2270 operatively linked to a sequence having at least 70% sequence identity to the sequence of SEQ ID NO: 3648.

In some embodiments, the sequence having at least 70% sequence identity to the sequence of SEQ ID NO: 1346 is operatively linked to the sequence having at least 70% sequence identity to the sequence of SEQ ID NO: 1349 via the sequence of SEQ ID NO: 3801.

In some embodiments, the sequence having at least 70% sequence identity to the sequence of SEQ ID NO: 1349 is operatively linked to the sequence having at least 70% sequence identity to the sequence of SEQ ID NO: 2270 via the sequence of SEQ ID NO: 3309.

In some embodiments, the sequence having at least 70% sequence identity to the sequence of SEQ ID NO: 2270 is operatively linked to the sequence having at least 70% sequence identity to the sequence of SEQ ID NO: 3648 via the sequence of SEQ ID NO: 3308.

In some embodiments, the second polypeptide chain comprises the sequence of SEQ ID NO: 1346 operatively linked to the sequence of SEQ ID NO: 1349 operatively linked to the sequence of SEQ ID NO: 2270 operatively linked to the sequence of SEQ ID NO: 3648.

In some embodiments, the sequence of SEQ ID NO: 1346 is operatively linked to the sequence of SEQ ID NO: 1349 via the sequence of SEQ ID NO: 3801.

In some embodiments, the sequence of SEQ ID NO: 1349 is operatively linked to the sequence of SEQ ID NO: 2270 via the sequence of SEQ ID NO: 3309.

In some embodiments, the sequence of SEQ ID NO: 2270 is operatively linked to the sequence of SEQ ID NO: 3648 via the sequence of SEQ ID NO: 3308.

In some embodiments, the second polypeptide chain comprises a sequence having at least 70% sequence identity to the sequence of SEQ ID NO: 3800.

In some embodiments, the second polypeptide chain comprises the sequence of SEQ ID NO: 3800.

In some embodiments, the multifunctional molecule consists of a first polypeptide chain and a second polypeptide chain, wherein the first polypeptide chain comprises the tumor-associated antigen binding moiety linked to an Fc region, wherein the tumor-associated antigen binding moiety is an scFv that binds to a tumor-associated antigen, and the second polypeptide chain comprises the sequence of SEQ ID NO 3800.

In some embodiments, the multifunctional molecule consists of a first polypeptide chain and a second polypeptide chain, wherein the first polypeptide chain comprises the tumor-associated antigen binding moiety linked to a sequence having at least 75% sequence identity to the sequence of SEQ ID NO: 3649 or SEQ ID NO: 3792, wherein the tumor-associated antigen binding moiety is an scFv that binds to a tumor-associated antigen, and the second polypeptide chain comprises the sequence of SEQ ID NO 3800.

In some embodiments, the multifunctional molecule consists of a first polypeptide chain and a second polypeptide chain, wherein the first polypeptide chain comprises the tumor-associated antigen binding moiety linked to the sequence of SEQ ID NO. 3649 or SEQ ID NO: 3792, wherein the tumor-associated antigen binding moiety is an scFv that binds to a tumor-associated antigen, and the second polypeptide chain comprises the sequence of SEQ ID NO 3800.

In some embodiments, the multifunctional molecule consists of a first polypeptide chain, a second polypeptide chain, and a third polypeptide chain, wherein the first polypeptide chain comprises a first portion of the tumor-associated antigen binding moiety linked to an Fc region, wherein the tumor-associated antigen binding moiety is an Fab that binds to a tumor-associated antigen: the second polypeptide chain comprises the sequence of SEQ ID NO 3800; and the third polypeptide chain comprises a second portion of the tumor-associated antigen binding moiety.

In some embodiments, the multifunctional molecule consists of a first polypeptide chain, a second polypeptide chain, and a third polypeptide chain, wherein the first polypeptide chain comprises a first portion of the tumor-associated antigen binding moiety linked to a sequence having at least 75% sequence identity to the sequence of SEQ ID NO: 3649 or SEQ ID NO: 3792, wherein the tumor-associated antigen binding moiety is an Fab that binds to a tumor-associated antigen; the second polypeptide chain comprises the sequence of SEQ ID NO 3800; and the third polypeptide chain comprises a second portion of the tumor-associated antigen binding moiety.

In some embodiments, the multifunctional molecule consists of a first polypeptide chain, a second polypeptide chain, and a third polypeptide chain, wherein the first polypeptide chain comprises a first portion of the tumor-associated antigen binding moiety linked to the sequence of SEQ ID NO: 3649 or SEQ ID NO: 3792, wherein the tumor-associated antigen binding moiety is an Fab that binds to a tumor-associated antigen; the second polypeptide chain comprises the sequence of SEQ ID NO 3800; and the third polypeptide chain comprises a second portion of the tumor-associated antigen binding moiety.

In some embodiments, the tumor-associated antigen binding moiety binds to a cancer antigen selected from the group consisting of CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA). Receptor tyrosine kinase-like orphan receptor 1 (ROR1), Fms-Like Tyrosine Kinase 3 (FLT3), Tumor-associated glycoprotein 72 (TAG72), CD38, CD44v6. Carcinoembryonic antigen (CEA). Epithelial cell adhesion molecule (EPCAM). B7H3 (CD276), KIT (CD117). Interleukin-13 receptor subunit alpha-2, mesothelin, Interleukin 11 receptor alpha (IL-11Ra), prostate stem cell antigen (PSCA). Protease Serine 21, vascular endothelial growth factor receptor 2 (VEGFR2), Lewis (Y) antigen, CD24. Platelet-derived growth factor receptor beta (PDGFR-beta). Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM). Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type. 9 (LMP2), glycoprotein 100 (gp100/pmel17), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3). G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1/LAGE-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, Survivin, telomerase, prostate carcinoma tumor antigen-1, melanoma antigen recognized by T cells 1. Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN). Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3). Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2). Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut bsp 70-2), CD79a, CD79b, CD72. Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), FcRH5, PDL1, CD47, prostate specific membrane antigen (PMSA), prostate-specific antigen (PSA), Ron Kinase, c-Met, Immature laminin receptor, TAG-72, BING-4, Calcium-activated chloride channel 2, Cyclin-B1, 9D7, Ep-CAM, EphA3, SAP-1, PRAME, SSX-2, Melan-A/MART-1, TRP1/gp75, MC1R, β-catenin, BRCA1/2, CDK4, CML66, Fibronectin. Ras, TGF-B receptor, AFP, ETA, MAGE, CA-125, BAGE, GAGE, CDC27, α actinin-4, gangliosides, MART-2, MUC2, MUM1, MUM2, MUM3, NA88-1, NPM, OA1, OGT, RCC, RUI1, RU12, SAGE, TRG, TSTA, L1-CAM, gpA33, GM2, VEGFR, Integrins, carbohydrates, TRAILR1, TRAILR2, RANKL, TGF-beta, hyaluronic acid, collagen, tenascin C, tenascin W, and immunoglobulin lambda-like polypeptide 1 (IGLL1).

In some embodiments, the tumor-associated antigen binding moiety binds to a cancer antigen selected from the group consisting of CD20, MSLN, gp75 (Tryp1), or any combination thereof.

In some embodiments, the multifunctional molecule is a polypeptide molecule.

In some embodiments, the tumor-associated antigen binding moiety comprises a VH comprising a HC CDR1, a HC CDR2 and a HC CDR3 comprising the sequences of SEQ ID NO: 524, SEQ ID NO: 525, and SEQ ID NO: 526, respectively, or a VH comprising a HC CDR1, a HC CDR2 and a HC CDR3 comprising the sequences of SEQ ID NO: 538, SEQ ID NO: 539, and SEQ ID NO: 540, respectively.

In some embodiments, the tumor-associated antigen binding moiety comprises a VL comprising a LC CDR1, a LC CDR2 and a LC CDR3 comprising the sequences of SEQ ID NO: 1168, SEQ ID NO: 1295, and SEQ ID NO: 528, respectively, or a VL comprising a LC CDR1, a LC CDR2 and a LC CDR3 comprising the sequences of SEQ ID NO: 562, SEQ ID NO: 563, and SEQ ID NO: 564, respectively.

In some embodiments, the tumor-associated antigen binding moiety comprises a VH comprising a HC CDR1, a HC CDR2 and a HC CDR3 comprising the sequences of SEQ ID NO: 524, SEQ ID NO: 525, and SEQ ID NO: 526, respectively, and a VL comprising a LC CDR1, a LC CDR2 and a LC CDR3 comprising the sequences of SEQ ID NO: 1168, SEQ ID NO: 1295, and SEQ ID NO: 528, respectively.

In some embodiments, the tumor-associated antigen binding moiety comprises a VH comprising a HC CDR1, a HC CDR2 and a HC CDR3 comprising the sequences of SEQ ID NO: 538, SEQ ID NO: 539, and SEQ ID NO: 540, respectively, and a VL comprising a LC CDR1, a LC CDR2 and a LC CDR3 comprising the sequences of SEQ ID NO: 562, SEQ ID NO: 563, and SEQ ID NO: 564, respectively.

In some embodiments, the tumor-associated antigen binding moiety comprises a VH comprising a sequence having at least 70% sequence identity to the sequence of SEQ ID NO: 523 or the sequence of SEQ ID NO: 537.

In some embodiments, the tumor-associated antigen binding moiety comprises a VL comprising a sequence having at least 70% sequence identity to the sequence of SEQ ID NO: 527 or the sequence of SEQ ID NO: 561.

In some embodiments, the tumor-associated antigen binding moiety comprises a VH comprising the sequence of SEQ ID NO: 523 or the sequence of SEQ ID NO: 537.

In some embodiments, the tumor-associated antigen binding moiety comprises a VL comprising the sequence of SEQ ID NO: 527 or the sequence of SEQ ID NO: 561.

In some embodiments, the tumor-associated antigen binding moiety comprises a VH comprising a sequence having at least 70% sequence identity to the sequence of SEQ ID NO: 523 and a VL comprising a sequence having at least 70% sequence identity to the sequence of SEQ ID NO: 527.

In some embodiments, the tumor-associated antigen binding moiety comprises a VH comprising the sequence of SEQ ID NO: 523 and a VL comprising the sequence of SEQ ID NO: 527.

In some embodiments, the tumor-associated antigen binding moiety comprises a VH comprising a sequence having at least 70% sequence identity to the sequence of SEQ ID NO: 537 and a VL comprising a sequence having at least 70% sequence identity to the sequence of SEQ ID NO: 561.

In some embodiments, the tumor-associated antigen binding moiety comprises a VH comprising the sequence of SEQ ID NO: 537 and a VL comprising the sequence of SEQ ID NO: 561.

In some embodiments, the tumor-associated antigen binding moiety comprises a VH comprising a HC CDR1, a HC CDR2 and a HC CDR3 comprising the sequences of SEQ ID NO: 581, SEQ ID NO. 582, and SEQ ID NO: 583, respectively.

In some embodiments, the tumor-associated antigen binding moiety comprises a VL comprising a LC CDR1, a LC CDR2 and a LC CDR3 comprising the sequences of SEQ ID NO: 586, SEQ ID NO: 587, and SEQ ID NO: 588, respectively.

In some embodiments, the tumor-associated antigen binding moiety comprises a VH comprising a HC CDR1, a HC CDR2 and a HC CDR3 comprising the sequences of SEQ ID NO: 581, SEQ ID NO: 582, and SEQ ID NO: 583, respectively, and a VL comprising a LC CDR1, a LC CDR2 and a LC CDR3 comprising the sequences of SEQ ID NO: 586, SEQ ID NO: 587, and SEQ ID NO: 588, respectively.

In some embodiments, the tumor-associated antigen binding moiety comprises a VH comprising a sequence having at least 70% sequence identity to the sequence of SEQ ID NO: 580.

In some embodiments, the tumor-associated antigen binding moiety comprises a VL comprising a sequence having at least 70% sequence identity to the sequence of SEQ ID NO: 585.

In some embodiments, the tumor-associated antigen binding moiety comprises a VH comprising the sequence of SEQ ID NO: 580.

In some embodiments, the tumor-associated antigen binding moiety comprises a VL comprising the sequence of SEQ ID NO. 585.

In some embodiments, the tumor-associated antigen binding moiety comprises a VH comprising a sequence having at least 70% sequence identity to the sequence of SEQ ID NO: 580 and a VL comprising a sequence having at least 70% sequence identity to the sequence of SEQ ID NO: 585.

In some embodiments, the tumor-associated antigen binding moiety comprises a VH comprising the sequence of SEQ ID NO: 580 and a VL comprising the sequence of SEQ ID NO: 585.

In some embodiments, the TCRβV-binding moiety comprises a VH comprising a HC CDR1, a HC CDR2 and a HC CDR3 comprising the sequences of SEQ ID NO: 3650, SEQ ID NO: 3651, and SEQ ID NO: 5, respectively, or a VH comprising a HC CDR1, a HC CDR2 and a HC CDR3 comprising the sequences of SEQ ID NO: 542, SEQ ID NO. 543, and SEQ ID NO: 544, respectively.

In some embodiments, the TCRβV-binding moiety comprises a VL comprising a LC CDR1, a LC CDR2 and a LC CDR3 comprising the sequences of SEQ ID NO: 3655, SEQ ID NO: 3653, and SEQ ID NO: 8, respectively, or a VL comprising a LC CDR1, a LC CDR2 and a LC CDR3 comprising the sequences of SEQ ID NO: 545, SEQ ID NO: 546, and SEQ ID NO: 547, respectively.

In some embodiments, the TCRβV-binding moiety comprises a VH comprising a HC CDR1, a HC CDR2 and a HC CDR3 comprising the sequences of SEQ ID NO: 3650, SEQ ID NO: 3651, and SEQ ID NO: 5, respectively, and a VL comprising a LC CDR1, a LC CDR2 and a LC CDR3 comprising the sequences of SEQ ID NO: 3655, SEQ ID NO: 3653, and SEQ ID NO: 8, respectively.

In some embodiments, the TCRβV-binding moiety comprises a VH comprising a HC CDR1, a HC CDR2 and a HC CDR3 comprising the sequences of SEQ ID NO: 542, SEQ ID NO: 543, and SEQ ID NO: 544, respectively, and a VL comprising a LC CDR1, a LC CDR2 and a LC CDR3 comprising the sequences of SEQ ID NO: 545, SEQ ID NO: 546, and SEQ ID NO: 547, respectively.

In some embodiments, the TCRβV-binding moiety comprises a VH comprising a sequence having at least 70% sequence identity to the sequence of SEQ ID NO: 1346 or the sequence of SEQ ID NO: 541.

In some embodiments, the TCRβV-binding moiety comprises a VL comprising a sequence having at least 70% sequence identity to the sequence of SEQ ID NO: 1349 or the sequence of SEQ ID NO: 3527.

In some embodiments, the TCRβV-binding moiety comprises a VH comprising the sequence of SEQ ID NO: 1346 or the sequence of SEQ ID NO: 541.

In some embodiments, the TCRβV-binding moiety comprises a VL comprising the sequence of SEQ ID NO: 1349 or the sequence of SEQ ID NO: 3527.

In some embodiments, the TCRβV-binding moiety comprises a VH comprising a sequence having at least 70% sequence identity to the sequence of SEQ ID NO: 1346 and a VL comprising a sequence having at least 70% sequence identity to the sequence of SEQ ID NO: 1349.

In some embodiments, the TCRβV-binding moiety comprises a VH comprising the sequence of SEQ ID NO: 1346 and a VL comprising the sequence of SEQ ID NO. 1349.

In some embodiments, the TCRβV-binding moiety comprises a VH comprising a sequence having at least 70% sequence identity to the sequence of SEQ ID NO: 541 and a VL comprising a sequence having at least 70% sequence identity the sequence of SEQ ID NO: 3527.

In some embodiments, the TCRβV-binding moiety comprises a VH comprising the sequence of SEQ ID NO: 541 and a VL comprising the sequence of SEQ ID NO: 3527.

In some embodiments, the TCRβV-binding moiety comprises an scFv comprising the sequence of SEQ ID NO: 1346 operatively linked to the sequence of SEQ ID NO: 1349 via a linker comprising the sequence of SEQ ID NO. 3801.

In some embodiments, the TCRβV-binding moiety comprises an scFv comprising the sequence of SEQ ID NO: 541 operatively linked to the sequence of SEQ ID NO: 3527 via a linker comprising the sequence of SEQ ID NO: 3801.

In some embodiments, the TCRβV-binding moiety comprises an scFv comprising a sequence having at least 70% sequence identity to the sequence of SEQ ID NO: 1331 or the sequence of SEQ ID NO: 1376.

In some embodiments, the TCRβV-binding moiety comprises an scFv comprising the sequence of SEQ ID NO: 1331 or the sequence of SEQ ID NO: 1376.

In some embodiments, the multifunctional molecule consists of a first polypeptide chain, a second polypeptide chain, and a third polypeptide chain, wherein:
(i) the first polypeptide chain comprises the sequence of SEQ ID NO: 523, and the sequence of SEQ ID NO: 3649;
(ii) the second polypeptide chain comprises the sequence of SEQ ID NO: 1331, the sequence of SEQ ID NO: 2270, and the sequence of SEQ ID NO: 3648; and
(iii) the third polypeptide chain comprises the sequence of SEQ ID NO: 527, and the sequence of SEQ ID NO: 3644.

In some embodiments, the multifunctional molecule consists of a first polypeptide chain, a second polypeptide chain, and a third polypeptide chain, wherein:
(i) the first polypeptide chain comprises the sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO: 2270 operatively linked to the sequence of SEQ ID NO: 3648; and
(iii) the third polypeptide chain comprises the sequence of SEQ ID NO: 527 operatively linked to the sequence of SEQ ID NO: 3644.

In some embodiments, the multifunctional molecule consists of a first polypeptide chain, a second polypeptide chain, and a third polypeptide chain, wherein:
(i) the first polypeptide chain comprises a sequence having at least 70% sequence identity to the sequence of SEQ ID NO: 501;
(ii) the second polypeptide chain comprises a sequence having at least 70% sequence identity to the sequence of SEQ ID NO: 3800; and
(iii) the third polypeptide chain comprises a sequence having at least 70% sequence identity to the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 501;
(ii) the second polypeptide chain comprises the sequence of SEQ ID NO: 3800; and
(iii) the third polypeptide chain comprises the sequence of SEQ ID NO: 502.

In some embodiments, the multifunctional molecule consists of a first polypeptide chain, a second polypeptide chain, and a third polypeptide chain, wherein:
(i) the first polypeptide chain comprises a first sequence of SEQ ID NO: 523, a second sequence of SEQ ID NO: 523, and the sequence of SEQ ID NO: 3649;
(ii) the second polypeptide chain comprises the sequence of SEQ ID NO: 1331, the sequence of SEQ ID NO: 2270, and the sequence of SEQ ID NO: 3648; and
(iii) the third polypeptide chain comprises the sequence of SEQ ID NO: 527, and the sequence of SEQ ID NO: 3644.

In some embodiments, the multifunctional molecule consists of a first polypeptide chain, a second polypeptide chain, and a third polypeptide chain, wherein:
(i) the first polypeptide chain comprises the first sequence of SEQ ID NO: 523 operatively linked to the second sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO: 2270 operatively linked to the sequence of SEQ ID NO: 3648; and
(iii) the third polypeptide chain comprises the sequence of SEQ ID NO: 527 operatively linked to the sequence of SEQ ID NO: 3644.

In some embodiments, the multifunctional molecule consists of a first polypeptide chain, a second polypeptide chain, and a third polypeptide chain, wherein:
(i) the first polypeptide chain comprises a sequence having at least 70% sequence identity to the sequence of SEQ ID NO: 590;
(ii) the second polypeptide chain comprises a sequence having at least 70% sequence identity to the sequence of SEQ ID NO: 3800; and
(iii) the third polypeptide chain comprises a sequence having at least 70% sequence identity to the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 590;
(ii) the second polypeptide chain comprises the sequence of SEQ ID NO: 3800; and
(iii) the third polypeptide chain comprises the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 537, and the sequence of SEQ ID NO: 559;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 561, and the sequence of SEQ ID NO: 558.

In some embodiments, the multifunctional molecule consists of a first polypeptide chain, a second polypeptide chain, and a third polypeptide chain, wherein:
(i) the first polypeptide chain comprises the sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558.

In some embodiments, the multifunctional molecule consists of a first polypeptide chain, a second polypeptide chain, and a third polypeptide chain, wherein:
(i) the first polypeptide chain comprises a sequence having at least 70% sequence identity to the sequence of SEQ ID NO: 509;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 509;
(ii) the second polypeptide chain comprises the sequence of SEQ ID NO: 511; and
(iii) the third polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises a first sequence of SEQ ID NO: 537, a second sequence of SEQ ID NO: 537, and the sequence of SEQ ID NO: 559;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 561, and the sequence of SEQ ID NO: 558.

In some embodiments, the multifunctional molecule consists of a first polypeptide chain, a second polypeptide chain, and a third polypeptide chain, wherein:
(i) the first polypeptide chain comprises the first sequence of SEQ ID NO: 537 operatively linked to the second sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558.

In some embodiments, the multifunctional molecule consists of a first polypeptide chain, a second polypeptide chain, and a third polypeptide chain, wherein:
(i) the first polypeptide chain comprises a sequence having at least 70% sequence identity to the sequence of SEQ ID NO: 512;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 512;
(ii) the second polypeptide chain comprises the sequence of SEQ ID NO: 511; and
(iii) the third polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 580, and the sequence of SEQ ID NO: 584;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 585, and the sequence of SEQ ID NO: 3528.

In some embodiments, the multifunctional molecule consists of a first polypeptide chain, a second polypeptide chain, and a third polypeptide chain, wherein:
(i) the first polypeptide chain comprises the sequence of SEQ ID NO: 580 operatively linked to the sequence of SEQ ID NO: 584;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 585 operatively linked to the sequence of SEQ ID NO: 3528.

In some embodiments, the multifunctional molecule consists of a first polypeptide chain, a second polypeptide chain, and a third polypeptide chain, wherein:
(i) the first polypeptide chain comprises a sequence having at least 70% sequence identity to the sequence of SEQ ID NO: 519;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 520.

In some embodiments, the multifunctional molecule consists of a first polypeptide chain, a second polypeptide chain, and a third polypeptide chain, wherein:
(i) the first polypeptide chain comprises the sequence of SEQ ID NO: 519;
(ii) the second polypeptide chain comprises the sequence of SEQ ID NO: 511; and
(iii) the third polypeptide chain comprises the sequence of SEQ ID NO: 520.

In some embodiments, the multifunctional molecule is a multispecific molecule.

In another aspect, provided herein is a polynucleotide comprising a sequence encoding the multifunctional molecule as provided herein.

In some embodiments, the polynucleotide is an isolated nucleic acid molecule.

In another aspect, provided herein is a vector comprising one or more of the polynucleotide as provided herein.

In another aspect, provided herein is a cell comprising the polynucleotide as provided herein, or the vector as provided herein.

In another aspect, provided herein is a pharmaceutical composition comprising the multifunctional molecule as provided herein, the polynucleotide as provided herein, the vector as provided herein, or the cell as provided herein, and a pharmaceutically acceptable carrier, excipient, or diluent.

In another aspect, provided herein is a method of treating a condition or disease in a subject in need therefor comprising administering to the subject a therapeutically effective amount of the multifunctional molecule as provided herein, the polynucleotide as provided herein, the vector as provided herein, the cell as provided herein, the pharmaceutical composition as provided herein, or any combination thereof, wherein the administering is effective to treat the condition or disease in the subject.

In some embodiments, the condition or disease is cancer.

In some embodiments, the cancer is a solid tumor, a hematological cancer, a metastatic cancer, a soft tissue tumor, or any combination thereof.

In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and any combination thereof.

In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and any combination thereof.

In some embodiments, the T-cell lymphoma is peripheral T-cell lymphoma.

In some embodiments, the cancer is characterized by a cancer antigen present on the cancer.

In some embodiments, the cancer antigen is a tumor antigen, a stromal antigen, or a hematological antigen.

In some embodiments, the cancer antigen is selected from the group consisting of CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM). B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type. 9 (LMP2), glycoprotein 100 (gp100/pmel17), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1/LAGE-1), Cancer/testis antigen 2 (LAGE-1a). Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein. Survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene). N-Acetyl glucosaminyl-transferase V (NA17), paired box protein Pax-3 (PAX3). Androgen receptor. Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like. Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), FcRH5, PDL1, CD47, prostate specific membrane antigen (PMSA), prostate-specific antigen (PSA), Ron Kinase, c-Met, Immature laminin receptor, TAG-72, BING-4, Calcium-activated chloride channel 2, Cyclin-B1, 9D7, Ep-CAM, EphA3, SAP-1, PRAME, SSX-2, Melan-A/MART-1, TRP1/gp75, MC1R, β-catenin, BRCA1/2, CDK4, CML66, Fibronectin, Ras, TGF-B receptor, AFP, ETA, MAGE, CA-125, BAGE, GAGE, CDC27, α actinin-4, gangliosides, MART-2, MUC2, MUM1, MUM2, MUM3, NA88-1, NPM, OA1, OGT, RCC, RU11, RU12, SAGE, TRG, TSTA, L1-CAM, gpA33, GM2, VEGFR, Integrins, carbohydrates, TRAILR1, TRAILR2, RANKL, TGF-beta, hyaluronic acid, collagen, tenascin C, tenascin W, and immunoglobulin lambda-like polypeptide 1 (IGLL1).

In some embodiments, the method as provided herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional molecule as provided herein, the polynucleotide as provided herein, the vector as provided herein, the cell as provided herein, the pharmaceutical composition as provided herein, sequentially, simultaneously, or concurrently.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-IT depict exemplary embodiments of multifunctional molecules as described herein. FIGS. 1A, 1B and 1C depict exemplary embodiments of multifunctional molecules containing multiple, e.g., two, molecules of an exemplary cytokine, interleukin-2 (IL-2), linked to an antibody molecule that binds to a T cell receptor beta variable region (TCRβV) (“anti-TCRβV antibody molecule”). FIGS. 1D, 1E and IF depict exemplary embodiments of multifunctional molecules containing a single molecule of an exemplary cytokine, IL-2, linked to an anti-TCRβV antibody molecule. FIGS. 1G, 1H, 1I, and 1J depict exemplary embodiments of multifunctional molecules containing an exemplary cytokine, IL-2, linked to a first dimerization module. FIGS. 1K, 1L and 1M depict exemplary embodiments of multifunctional molecules containing an exemplary dimerization module, e.g., an Fc region comprising a N297A mutation, and multiple. e.g., two, molecules of an exemplary cytokine, IL-2, linked to an anti-TCRβV antibody molecule. FIGS. 1N, 1O and 1P depict exemplary embodiments of multifunctional molecules containing an exemplary dimerization module, e.g., an Fc region comprising a N297A mutation (Knob-in-hole), and a single molecule of an exemplary cytokine, IL-2, linked to an anti-TCRβV antibody molecule. FIGS. 1Q, 1R, 1S and 1T depict exemplary embodiments of multifunctional molecules containing an exemplary dimerization module. e.g., an Fc region comprising a N297A mutation (Knob-in-hole), and an exemplary cytokine, IL-2, linked to the exemplary dimerization module.

FIGS. 2A-2B shows the alignment of the Antibody A source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 2A shows VH sequences for murine Antibody A (SEQ ID NO: 1) and humanized Antibody A-H (SEQ ID NO: 9). FIG. 2B shows VL sequences for murine Antibody A (SEQ ID NO: 2) and humanized Antibody A-H (SEQ ID NO: 10 and SEQ ID NO: 11).

FIGS. 3A-3B shows the alignment of the Antibody B source mouse VH and VL framework 1, CDR 1, framework 2, CDR 2, framework 3, CDR3, and framework 4 regions with their respective humanized sequences. Kabat CDRs are shown in bold. Chothia CDRs are shown in italics, and combined CDRs are shown in boxes. The framework positions that were back mutated are double underlined. FIG. 3A shows the VH sequence for murine Antibody B (SEQ ID NO: 15) and humanized VH sequences B-H.1A to B-H.1C (SEQ ID NOs: 23-25). FIG. 3B shows the VL sequence for murine Antibody B (SEQ ID NO: 16) and humanized VL sequences B-H.1D to B-H.1H (SEQ ID NOs: 26-30).

FIG. 4 depicts the phylogenetic tree of TCRβV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily I: TCRβ V18; Subfamily J: TCRβ V9; Subfamily K: TCRβ V13; Subfamily L: TCRβ V4; Subfamily M: TCRβ V3; Subfamily N: TCRβ V2; Subfamily O: TCRβ V15; Subfamily P: TCRβ V30; Subfamily Q: TCRβ V19; Subfamily R: TCRβ V27; Subfamily S: TCRβ V28; Subfamily T: TCRβ V24; Subfamily U: TCRβ V20; Subfamily V: TCRβ V25; and Subfamily W: TCRβ V29 subfamily. Subfamily members are described in detail herein in the Section titled “TCR beta V (TCRβV)”.

FIGS. 5A-5C show human CD3+ T cells activated by anti-TCR Vβ13.1 antibody (A-H.1) for 6-days. Human CD3+ T cells were isolated using magnetic-bead separation (negative selection) and activated with immobilized (plate-coated) anti-TCR Vβ13.1 (A-H.1) or anti-CD3ε (OKT3) antibodies at 100 nM for 6 days. FIG. 5A shows two scatter plots (left: activated with OKT3; and right: activated with A-H.1) of expanded T cells assessed for TCR Vβ13.1 surface expression using anti-TCR Vβ13.1 (A-H.1) followed by a secondary fluorochrome-conjugated antibody for flow cytometry analysis. FIG. 5B shows percentage (%) of TCR Vβ13.1 positive T cells activated by anti-TCR Vβ13.1 (A-H.1) or anti-CD3e (OKT3) plotted against total T cells (CD3+). FIG. 5C shows relative cell count acquired by counting the number of events in each T cell subset gate (CD3 or TCR Vβ13.1) for 20 seconds at a constant rate of 60 μl/min. Data shown as mean value from 3 donors.

FIGS. 6A-6B show cytolytic activity of human CD3+ T cells activated by anti-TCR Vβ13.1 antibody (A-H.1) against transformed cell line RPMI 8226. FIG. 6A depicts target cell lysis of human CD3+ T cells activated with A-H.1 or OKT3. Human CD3+ T cells were isolated using magnetic-bead separation (negative selection) and activated with immobilized (plate-coated) A-H.1 or OKT3 at the indicated concentrations for 4 days prior to co-culture with RPMI 8226 cells at a (E:T) ratio of 5:1 for 2 days. Samples were next analyzed for cell lysis of RPMI 8226 cells by FACS staining for CFSE/CD138-labeled, and membrane-impermeable DNA dyes (DRAQ7) using flow cytometry analysis FIG. 6B shows target cell lysis of human CD3+ T cells activated with A-H.1 or OKT3 incubated with RPMI-8226 at a (E:T) ratio of 5:1 for 6 days followed by cell lysis analysis of RPMI 8226 cells as described above. Percentage (%) target cell lysis was determined by normalizing to basal target cell lysis (i.e. without antibody treatment) using the following formula. [(x−basal)/(100%−basal), where x is cell lysis of sample]. Data shown is a representative of n=1 donor.

FIGS. 7A-7B show IFNγ production by human PBMCs activated with the indicated antibodies. Human PBMCs were isolated from whole blood from the indicated number of donors, followed by solid-phase (plate-coated) stimulation with the indicated antibodies at 100 Nm. Supernatant was collected on Days 1, 2, 3, 5, or 6. FIG. 7A is a graph comparing the production of IFNγ in human PBMCs activated with the antibodies indicated activated with anti-TCR Vβ13.1 antibodies (A-H.1 or A-H.2) or anti-CD3e antibodies (OKT3 or SP34-2) on Day 1, 2, 3, 5, or 6 post-activation. FIG. 7B shows IFNγ production in human PBMCs activated with the antibodies indicated activated with the indicated anti-TCR Vβ13.1 antibodies or anti-CD3e antibody (OKT3) on Day 1, 2, 3, 5, or 6 post-activation.

FIGS. 8A-8B show IL-2 production by human PBMCs activated with the indicated antibodies. A similar experimental setup as described for FIGS. 7A-7B was used.

FIGS. 9A-9B show IL-6 production by human PBMCs activated with the indicated antibodies. A similar experimental setup as described for FIGS. 7A-7B was used.

FIGS. 10A-10B show TNF-alpha production by human PBMCs activated with the indicated antibodies. A similar experimental setup as described for FIGS. 7A-7B was used.

FIGS. 11A-11B show IL-1beta production by human PBMCs activated with the indicated antibodies. A similar experimental setup as described for FIGS. 7A-7B was used.

FIGS. 12A-12B are graphs showing delayed kinetics of IFNγ secretion in human PMBCs activated by anti-TCR Vβ13.1 antibody A-H.1 when compared to PBMCs activated by anti-CD3e antibody OKT3. FIG. 12A shows IFNγ secretion data from 4 donors. FIG. 12B shows IFNγ secretion data from 4 additional donors. Data shown is representative of n=8 donors.

FIG. 13 depicts increased CD8+ TSCM and Temra T cell subsets in human PBMCs activated by anti-TCR Vβ13.1 antibodies (A-H.1 or A-H.2) compared to PBMCs activated by anti-CD3e antibodies (OKT3 or SP34-2).

FIGS. 14A-14F show characterization of an anti-TCRVb antibody. FIG. 14A is a graph depicting proliferation of T cells activated with anti-CD3 (OKT3) antibody or anti-TCRVb antibody. FIG. 14B shows selective expansion of CD45RA+ effector memory CD8+ and CD4+ T cells (TEMRA) cells with anti-TCRVb antibodies. Tn=naïve T cell: Tscm=stem cell memory T cell: Tcm=central memory T cell; Tem=effector memory T cell: Temra=effector memory CD45RA+ T cell. FIG. 14C is a graph showing IFN-g secretion by PBMCs stimulated with an anti-TCRVb antibody, or anti-CD3 antibodies. FIG. 14D shows target cell lysis by T cells stimulated with an anti-TCRVb antibody, or anti-CD3 antibodies. Cells were stimulated for 4 days followed by 2 days incubation with multiple myeloma target cells for assessment of cell killing. FIG. 14E is a graph showing perforin secretion by T cells stimulated with an anti-TCRVb antibody, or an anti-CD3 antibody. Perforin was analyzed by FACS staining in TCRVB-positive and TCRVB-negative T cells in PBMCs after 5 days of stimulation with 100 ng/ml plate-bound antibody. FIG. 14F is a graph showing Granzyme B by T cells stimulated with an anti-TCRVb antibody, or an anti-CD3 antibody. Granzyme B was analyzed by FACS staining in TCRVB-positive and TCRVB-negative T cells in PBMCs after 5 days of stimulation with 100 ng/ml plate-bound antibody.

FIGS. 15A-15B show production of IL-2 and IL-15 and expansion of human NK cells by stimulation of PBMCs with anti-TCRVb antibody for 6 days at a dose of 100 nM. FIG. 15A shows secretion of IL-2 or IL-15 in T cells stimulated with an anti-TCRVb antibody, or anti-CD3 antibodies. FIG. 15B depicts flow cytometry dot plots showing NKp46 staining vs CD56 antibody staining in cells stimulated with an anti-TCRVb antibody or an anti-CD3 antibody or a control sample.

FIGS. 16A-16C show secretion of cytokines in PBMCs stimulated with an anti-TCRVb antibody, or anti-CD3 antibodies.

FIGS. 17A-17B show killing of MM cells by dual targeting BCMA-TCRvb antibody molecules. FIG. 17A shows in vitro killing by one of the following dual-targeting antibody molecules: BCMA-TCRVb (Molecule I), BCMA-CD3, or Control-TCRVb; or an isotype control. FIG. 17B shows in vivo killing of MM cells by a dual-targeting BCM-TCRVb antibody (Molecule I).

FIG. 18 shows lysis of MM target cells with a dual targeting antibody (Molecule E) which recognized FcRH5 on one arm and TCRVb on the other arm.

FIGS. 19A-19B demonstrate cytokine production from human PBMCs activated by anti-TCR Vβ8a antibodies (B-H.1) when compared to those activated by anti-CD3e antibodies (OKT3 or SP34-2). FIG. 19A shows that human PBMCs activated by anti-TCR Vβ8a antibodies (B-H.1) produce similar or reduced levels of IFNγ. FIG. 19B shows human PBMCs activated by anti-TCR Vβ8a antibodies (B-H.1) produce higher levels of IL-2 when compared to those activated by anti-CD3ε antibodies (OKT3 or SP34-2). Data shown is representative of n=6 donors.

FIGS. 20A-20C demonstrate cytokine production from human PBMCs activated by anti-TCR Vβ8a antibodies (B-H.1). Human PBMCs activated by anti-TCR Vβ8a antibodies (B-H.1) do not significantly produce IL-6 (FIG. 20A), IL1β (FIG. 20B), and less TNFα (FIG. 20C), when compared to PBMCs activated by anti-CD3e antibodies (OKT3 or SP34-2). Data shown is representative of n=6 donors.

FIGS. 21A-21E demonstrate cytokine production from human PBMCs activated by anti-TCRβV Antibody D antibody compared to control anti-CD3e antibody (OKT3). FIG. 21A shows that human PBMCs activated by anti-TCRβV Antibody D antibody produce similar or reduced levels of IFNγ. FIG. 21B shows human PBMCs activated by anti-TCRβV Antibody D antibody produce higher levels of IL-2 when compared to those activated by anti-CD3ε antibodies (OKT3). Human PBMCs activated by anti-TCRβV Antibody D antibody do not significantly produce IL-1beta (FIG. 21C), IL-6. (FIG. 21D), or TNFalpha (FIG. 21E) Data shown is representative of n=4 donors.

FIGS. 22A-22B demonstrate cytokine production from human PBMCs activated by anti-TCR Vβ5 antibody (Antibody E). FIG. 22A shows that human PBMCs activated by anti-TCR Vβ5 antibody produce similar or reduced levels of IFNγ compared to PBMCS activated by anti-CD3ε antibodies (OKT3 or SP34-2). FIG. 22B shows human PBMCs activated by the anti-TCR Vβ5 1 antibody produce higher levels of IL-2 when compared to those activated by anti-CD3e antibodies (OKT3 or SP34-2). Data shown is representative of n=4 donors.

FIGS. 23A-23D demonstrate cytokine production from human PBMCs activated by an anti-TCR Vβ5 antibody (Antibody E). Human PBMCs activated by anti-TCR Vβ5 antibody do not significantly produce IL-1beta (FIG. 23A), IL-6, (FIG. 23B). TNFalpha (FIG. 23C), or IL-10 (FIG. 23D) as compared to PBMCs activated by anti-CD3e antibodies (OKT3 or SP34-2). Data shown is representative of n=4 donors.

FIGS. 24A-24F demonstrate cytokine production from human PBMCs activated by a dual targeting (bispecific molecule) comprising an anti-TCRβV binding moiety and a BCMA binding moiety. FIG. 24A shows that human PBMCs activated by the bispecific molecule produce similar or reduced levels of IFNγ as PBMCS activated by anti-CD3e antibodies (OKT3). FIG. 24B shows human PBMCs activated by the bispecific molecule produce higher levels of IL-2 when compared to PBMCs activated by anti-CD3ε antibodies (OKT3). Human PBMCs activated by the bispecific molecule do not significantly produce IL-1beta (FIG. 24C), IL-6, (FIG. 24D). TNFalpha (FIG. 24E), or IL-10 (FIG. 24F). Data shown is representative of n=3 donors.

FIGS. 25A-25B show the structure and sequence of eight TCRβV proteins from seven different subfamilies: TCRβV6 subfamily (TCRβV6-5 and TCRβV6-4 are shown), TCRβV28 subfamily, TCRβV19 subfamily, TCRβV9 subfamily, TCRβV5 subfamily, TCRβV20 subfamily and TCRβV12 subfamily. FIG. 25A shows the structural alignment of the different TCRβV proteins. The circled area represents the outward facing region comprising the proposed binding site for the anti-TCRβV antibodies as described herein. FIG. 25B shows the amino acid sequence alignment of the proteins shown in FIG. 25A (SEQ ID NOS 3449-3456, respectively, in order of appearance). The various TCRβV proteins (from 7 different TCRβV subfamilies) have diverse sequences but share a conserved (similar) structure and function.

FIGS. 26A-26J show cytokine or chemokine secretion of PBMCs activated with anti-TCRVb antibodies (A-H.1, B-H.1), a bispecific molecule comprising an anti-TCRVb antibody (Molecule H), control isotype (122) or anti-CD3e antibody (OKT3). Data shown is representative of n=2 donors and representative of 2 independent experiments.

FIGS. 27A-27H show cytokine or chemokine secretion of PBMCs activated with anti-TCRVb antibodies (A-H.1, B-H.1), a bispecific molecule comprising an anti-TCRVb antibody (Molecule H), control isotype (122) or anti-CD3e antibody (OKT3). Data shown is representative of n=2 donors and representative of 2 independent experiments.

FIGS. 28A-28L show cytokine or chemokine secretion of PBMCs activated with anti-TCRVb antibodies (A-H.1, B-H.1), a bispecific molecule comprising an anti-TCRVb antibody (Molecule H), control isotype (122) or anti-CD3e antibody (OKT3). Data shown is representative of n=2 donors and representative of 2 independent experiments.

FIG. 29 is a graph depicting mean tumor volume in NOD/SCID/IL-2Rγnull (NSG) mice engrafted with Raji-luc cells at days 10 to 28. The Star denotes PBMC implantation. Open triangles denote antibody treatment with the indicated antibodies.

FIGS. 30A-30F are graphs showing cytokine secretion stimulated by anti-TRBC1 (Antibody F) or anti-CD3 (OKT3) at Days 2 and 5. Cytokines examined include: IFNγ (FIG. 30A), IL-2 (FIG. 30B), IL-1β (FIG. 30C), IL-6 (FIG. 30D), IL-10 (FIG. 30E), and TNFα (FIG. 31F).

FIG. 31 is a FACS plot showing the expansion of TCRvb 6-5+ T cells over 8 days using anti-TCRvb 6-5 v1.

FIG. 32 is a bar graph showing the expansion of TCRvb 6-5+ CD4+ T cells and TCRvb 6-5+ CD8+ T cells over 8 days using the anti-CD3ε antibody OKT3 (100 nM).

FIG. 33 is a bar graph showing the expansion of TCRvb 6-5+ CD4+ T cells and TCRvb 6-5+ CD8+ T cells over 8 days using the anti-TCRvb 6-5 v1 antibody (100 nM).

FIG. 34 is a FACS plot showing the showing the expansion of TCRvb 6-5+ T cells over 8 days using anti-TCRvb 6-5 v1 or the anti-CD3ε antibody OKT3.

FIG. 35A is a bar graph showing the percentage of TCRβV 6-5+ T cells in PBMC cultures after 8 days of culture with the indicated antibody. Data for 5 replicates are shown. FIG. 35B is a bar graph showing the percentage of TCRβV 6-5+ T cells in purified T cell cultures after 8 days of culture with the indicated antibody. Data for 5 replicates are shown.

FIG. 36A is a bar graph showing the relative count of TCRβV 6-5+ T cells in PBMC culture after 8 days of culture with the indicated antibody. FIG. 36B is a bar graph showing the relative count of TCRβV 6-5+ T cells in PBMC culture after 8 days of culture with the indicated antibody.

FIG. 37A is a bar graph showing the relative count of TCRβV 6-5+ T cells in a purified T cell culture after 8 days of culture with the indicated antibody. FIG. 37B is a bar graph showing the relative count of TCRβV 6-5+ T cells in a purified T cell culture after 8 days of culture with the indicated antibody.

FIG. 38 is a line graph showing the total CD3+ T cell count (fold increase) after 8 days of T cell culture with either the anti-CD3ε antibody OKT3 or the anti-TCRvb 6-5 v1 antibody.

FIG. 39 is a series of line graphs showing the kinetics of target cells by TCRβV 6-5 v1 activated T cells or anti-CD3ε (OKT3) activated T cells. T cells from three different donors were utilized (donor 6769, donor 9880, donor 5411).

FIG. 40A is a scatter plot showing the percent of target cell lysis by T cells by TCRβV 6-5 v1 activated T cells or anti-CD3ε (OKT3) activated T cells without T cell pre activation. The data is presented at day 6 of co-culture between target cells and effector T cells. FIG. 40B is a scatter plot showing the percent of target cell lysis by T cells by TCRβV 6-5 v1 activated T cells or anti-CD3ε (OKT3) activated T cells with 4 days of T cell pre activation. The data is presented at day 2 of co-culture between target cells and effector T cells (after 4 days of T cell pre-activation).

FIG. 41 is a scatter plot showing the percent of target cell lysis by T cells by TCRβV 6-5 v1 activated T cells or anti-CD3ε (OKT3) activated T cells with 4 days of T cell pre activation. The data is presented at day 2 of co-culture between target cells and effector T cells (after 4 days of T cell pre-activation).

FIG. 42 is a bar graph showing target cell lysis by T cells by TCRβV 6-5 v1 activated T cells or anti-CD3ε (OKT3) activated T cells (100 nM each antibody). The data includes seven replicates of each experimental condition.

FIG. 43 is a series of FACS plots that show the cell surface expression of CD3ε on CD4+ TCRβV 6-5⁻ or CD4+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 44 is a series of FACS plots that show the cell surface expression of CD3ε on CD8+ TCRβV 6-5⁻ or CD8+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 45 is a series of FACS plots that show the cell surface expression of TCRβV on CD4+ TCRβV 6-5⁻ or CD4+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 46 is a series of FACS plots that show the cell surface expression of TCRβV on CD8+ TCRβV 6-5⁻ or CD8+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 47A shows FACS plot of TCRβV 6-5⁺ cynomolgus T cell expansion either unstimulated (left) or stimulated with anti-TCRβV 6-5 v1 (right) 7 days post activation of cynomolgus PBMCs from Donor DW8N (fresh PBMC sample, male, age 8, weight 7.9 kgs) were used. FIG. 47B shows FACS plot of TCRβV 6-5⁺ cynomolgus T cell expansion either unstimulated (left) or stimulated with anti-TCRβV 6-5 v1 (right) 7 days post activation of cynomolgus PBMCs. PBMCs from Donor G709 (cryopreserved sample, male, age 6, weight 4.7 kgs) were used.

FIG. 48 shows FACS plot and corresponding microscopy images of TCRβV 6-5⁺ cynomolgus T cell expansion either unstimulated (left), stimulated with SP34-2 (anti-CD3ε antibody) (middle); or stimulated with anti-TCRβV 6-5 v1 (right) post activation of cryopreserved donor DW8N cynomolgus PBMCs. The microscopy images show the cell cluster formation (indicated by circles).

FIG. 49 shows a schematic of FACS plot showing the FACS gating/staining of PBMCs prior γδ T cell purification.

FIG. 50 shows a schematic of FACS plot showing the FACS gating/staining of purified γδ T cell population.

FIG. 51 show activation of purified γδ T cell population with anti-CD3ε antibody (SP34-2) (left) or anti-TCRβV antibody (anti-TCRβV 6-5 v1) (right).

FIG. 52A shows the release of IFNγ from purified γδ T cell populations activated with anti-CD3ε antibody (SP34-2), anti-TCRβV antibody (anti-TCRβV 6-5 v1), or unstimulated. FIG. 52B shows the release of TNFα from purified γδ T cell populations activated with anti-CD3ε antibody (SP34-2), anti-TCRβV antibody (anti-TCRβV 6-5 v1), or unstimulated. FIG. 52C shows the release of IL-2 from purified γδ T cell populations activated with anti-CD3ε antibody (SP34-2), anti-TCRβV antibody (anti-TCRβV 6-5 v1), or unstimulated. FIG. 52D shows the release of IL-17A from purified γδ T cell populations activated with anti-CD3ε antibody (SP34-2), anti-TCRβV antibody (anti-TCRβV 6-5 v1), or unstimulated. FIG. 52E shows the release of IL-1a from purified γδ T cell populations activated with anti-CD3ε antibody (SP34-2), anti-TCRβV antibody (anti-TCRβV 6-5 v1), or unstimulated. FIG. 52F shows the release of IL-1β from purified γδ T cell populations activated with anti-CD3ε antibody (SP34-2), anti-TCRβV antibody (anti-TCRβV 6-5 v1), or unstimulated FIG. 52G shows the release of IL-6 from purified γδ T cell populations activated with anti-CD3ε antibody (SP34-2), anti-TCRβV antibody (anti-TCRβV 6-5 v1), or unstimulated. FIG. 52H shows the release of IL-10 from purified γδ T cell populations activated with anti-CD3ε antibody (SP34-2), anti-TCRβV antibody (anti-TCRβV 6-5 v1), or unstimulated.

FIG. 53 shows the relative representations of all TCR alpha V segments (TRAV group of genes) and their variants (top), all TCR beta V segment 6-5 variants (TRBV6-5 gene) (bottom left), and all TCR beta V segments and variants excluding 6-5 (bottom right).

FIG. 54A is a FACS plot showing phenotypic markers of CD4+ T cells expanded with anti-TCRβV antibody (anti-TCRβV 6-5 v1). Defined phenotypes include TEMRA (top left). Naïve/TSCM (top right), TEM (bottom left), and TCM (bottom right). FIG. 54B is a FACS plot showing phenotypic markers of CD4+ T cells expanded with anti-CD3ε antibody (OKT3). Defined phenotypes include TEMRA (top left). Naïve/TSCM (top right), TEM (bottom left), and TCM (bottom right).

FIG. 55A is a FACS plot showing phenotypic markers of CD8+ T cells expanded with anti-TCRβV antibody (anti-TCRβV 6-5 v1). Defined phenotypes include TEMRA (top left), Naïve/TSCM (top right). TEM (bottom left), and TCM (bottom right). FIG. 55B is a FACS plot showing phenotypic markers of CD8+ T cells expanded with anti-CD3ε antibody (OKT3). Defined phenotypes include TEMRA (top left), Naïve/TSCM (top right). TEM (bottom left), and TCM (bottom right).

FIG. 56A is a bar graph showing the percentage of PD1 expressing CD4+ T cells from T cell cultures activated with anti-TCRβV antibody (anti-TCRβV 6-5 v1), anti-CD3ε antibody (OKT3), or unstimulated. FIG. 56B is a bar graph showing the percentage of PD1 expressing CD8+ T cells from T cell cultures activated with anti-TCRβV antibody (anti-TCRβV 6-5 v1), anti-CD3ε antibody (OKT3), or unstimulated.

FIG. 57A is a bar graph showing the expression of Ki-67 by CD4+ T cells from T cell cultures activated with anti-TCRβV antibody (anti-TCRβV 6-5 v1), anti-CD3ε antibody (OKT3), or unstimulated. FIG. 57B is a bar graph showing the expression of Ki-67 by CD8+ T cells from T cell cultures activated with anti-TCRβV antibody (anti-TCRβV 6-5 v1), anti-CD3ε antibody (OKT3), or unstimulated.

FIG. 58A is a FACS plot showing the percentage of TEMRA-like CD8+ T cells activated using anti-TCRβV antibody (anti-TCRβV 6-5 v1) that express CD57 (18.7%). FIG. 58B is a FACS plot showing the percentage of TEM-like CD8+ T cells activated using anti-CD3ε antibody (OKT3) that express CD57 (46.8%) and the percentage of TCM-like CD8+ T cells activated using anti-CD3ε antibody (OKT3) that express CD57 (18.9%).

FIG. 59 shows a series of FACS plots showing the expression of expression of CD27 and by CD4+ (top) or CD8+ (bottom) T cells from T cell cultures activated with anti-TCRβV antibody (anti-TCRβV 6-5 v1), anti-CD3ε antibody (OKT3), or unstimulated.

FIG. 60 shows a series of FACS plots showing the expression of expression of OX40, 41BB, and ICOS by CD4+ (top) or CD8+ (bottom) T cells from T cell cultures activated with anti-TCRβV antibody (anti-TCRβV 6-5 v1), anti-CD3ε antibody (OKT3), or unstimulated.

FIG. 61 shows a series of FACS plots showing the percentage of CD3+ (CD4 gated) TCRβV 6-5+ T cells 1, 2, 3, 4, 5, 6, and 8 days port activation with BCMA and the anti-TCR Vβ antibody anti-TCR Vβ 6-5 v1.

FIG. 62A shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 0 post activation. FIG. 62B shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 1 post activation FIG. 62C shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 2 post activation. FIG. 62D shows a series of FACS plots showing the percentage of CD4+ T cells expanded using isotype control (IgG1 N297A), anti-TCRβV (anti-TCR Vβ 6-5 v1), or anti-CD3E (OKT3) antibodies on day 3 post activation. FIG. 62E shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 4 post activation. FIG. 62F shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 5 post activation. FIG. 62G shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 6 post activation. FIG. 62H shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 8 post activation.

FIG. 63A is a bar graph showing ATP production from glycolysis of T cell cultures activated with the indicated antibodies. FIG. 63B is a bar graph showing ATP production from oxidative phosphorylation of T cell cultures activated with the indicated antibodies.

FIG. 64 is a line graph showing the oxygen consumption rate (OCR) of T cells from about 0 to 75 minutes activated with the indicated antibody.

FIG. 65A shows the oxygen consumption rate (OCR) of T cells activated with the indicated antibody during basal respiration. FIG. 65B shows the oxygen consumption rate (OCR) of T cells activated with the indicated antibody during maximal respiration. FIG. 65C shows the oxygen consumption rate (OCR) of T cells activated with the indicated antibody during spare respiratory capacity. FIG. 65D is a line graph indicates the areas of basal respiration and maximal respiration as shown in FIG. 64A and FIG. 64B, respectively.

FIG. 66A is a bar graph showing ATP production from glycolysis of T cell cultures activated with anti-TCRβV 6-5 v1 and re-stimulated with the indicated antibody. FIG. 66B is a bar graph showing ATP production from oxidative phosphorylation of T cell cultures activated with anti-TCRβV 6-5 v1 and re-stimulated with the indicated antibody.

FIGS. 67A-67G are graphs showing expression of IFNγ (FIG. 67A), TNFα (FIG. 67E), IL-1α (FIG. 67B), IL-1β (FIG. 67C), IL-6 (CRS and neurotoxicity associated cytokines) (FIG. 67D) with BHM1710 (anti TCRVB), a reduced affinity anti CD3 antibody (TB) and the SP34 anti CD3e antibody, IL-10 (FIG. 67F), IL-17A (FIG. 67G).

FIG. 68 is a FACS plot showing the percentage of NK cells expanded from T cell cultures activated with the indicated antibody.

FIG. 69 is a bar graph showing the number of NK cells expanded from T cell cultures activated with the indicated antibody.

FIG. 70 shows a series of FACS plots showing NK cell proliferation induced by T cell cultures activated with the indicated antibody.

FIG. 71 is a schematic showing an assay described in Example for determining NK cell mediated lysis of target K562 cells.

FIG. 72 is a bar graph showing the percent target cell lysis mediated by NK cells activated by PBMC's activated with the indicated antibody.

FIG. 73 shows a series of FACS plots showing the proliferation of NK cells from PBMC cultures activated/expanded with the indicated antibody (isotype control or OKT3). PBMCs from three donors (D1, D2, and D3) were analyzed.

FIG. 74 shows a series of FACS plots showing the proliferation of NK cells from PBMC cultures activated/expanded with the indicated antibody (anti-TCRvβ 12-3/4 v1 or anti-TCRvβ 12-3/4 v2). PBMCs from three donors (D1, D2, and D3) were analyzed.

FIG. 75 shows a series of FACS plots showing the proliferation of NK cells from PBMC cultures activated/expanded with the indicated antibody (anti-TCRvβ 12-3/4 v3 or SP34-2). PBMCs from three donors (D1, D2, and D3) were analyzed.

FIG. 76 is a bar graph showing the level of secreted IFNγ by T cells activated/expanded with the indicated antibody (anti-TCRβV 6-5 v1. OKT3, or SP34) and cultured with said antibody for the indicated number of days (1, 3, or 5).

FIG. 77 is a bar graph showing the level of secreted IL-2 by T cells activated/expanded with the indicated antibody (anti-TCRβV 6-5 v1, OKT3, or SP34) and cultured with said antibody for the indicated number of days (1, 3, or 5).

FIG. 78 is a bar graph showing the level of secreted IL-15 by T cells activated/expanded with the indicated antibody (anti-TCRβV 6-5 v1, OKT3, or SP34) and cultured with said antibody for the indicated number of days (1, 3, or 5).

FIG. 79 is a bar graph showing the level of secreted IL-1β by T cells activated/expanded with the indicated antibody (anti-TCRβV 6-5 v1. OKT3, or SP34) and cultured with said antibody for the indicated number of days (1, 3, or 5).

FIG. 80 is a bar graph showing the level of secreted IL-6 by T cells activated/expanded with the indicated antibody (anti-TCRβV 6-5 v1, OKT3, or SP34) and cultured with said antibody for the indicated number of days (1, 3, or 5).

FIG. 81 is a bar graph showing the level of secreted IL-10 by T cells activated/expanded with the indicated antibody (anti-TCRβV 6-5 v1, OKT3, or SP34) and cultured with said antibody for the indicated number of days (1, 3, or 5).

FIG. 82 is a bar graph showing the level of the indicated cytokine secreted by T cells activated/expanded with the indicated antibody (anti-TCRβV 6-5 v1 or SP34). The data includes use of 17 individual PBMC donors.

FIG. 83A is a bar graph showing the level of secreted IFNγ by T cells activated/expanded with the indicated antibody (anti-TCRβV 6-5 v1 or OKT3) and cultured with said antibody for the indicated number of days (1, 2, 3, 5, or 6). FIG. 83B is a bar graph showing the level of secreted IL-1β by T cells activated/expanded with the indicated antibody (anti-TCRβV 6-5 v1 or OKT3) and cultured with said antibody for the indicated number of days (1, 2, 3, 5, or 6). FIG. 83C is a bar graph showing the level of secreted IL-4 by T cells activated/expanded with the indicated antibody (anti-TCRβV 6-5 v1 or OKT3) and cultured with said antibody for the indicated number of days (1, 2, 3, 5, or 6). FIG. 83D is a bar graph showing the level of secreted IL-6 by T cells activated/expanded with the indicated antibody (anti-TCRβV 6-5 v1 or OKT3) and cultured with said antibody for the indicated number of days (1, 2, 3, 5, or 6). FIG. 83E is a bar graph showing the level of secreted IL-10 by T cells activated/expanded with the indicated antibody (anti-TCRβV 6-5 v1 or OKT3) and cultured with said antibody for the indicated number of days (1, 2, 3, 5, or 6). FIG. 83F is a bar graph showing the level of secreted TNFα by T cells activated/expanded with the indicated antibody (anti-TCRβV 6-5 v1 or OKT3) and cultured with said antibody for the indicated number of days (1, 2, 3, 5, or 6). FIG. 83G is a bar graph showing the level of secreted IL-2 by T cells activated/expanded with the indicated antibody (anti-TCRβV 6-5 v1 or OKT3) and cultured with said antibody for the indicated number of days (1, 2, 3, 5, or 6).

FIG. 84A is a bar graph showing the level of secreted IFNγ by T cells activated/expanded with the indicated antibody (anti-TCRβV 6-5 v1, anti-TCRβV 6-5 v1. OKT3, SP34-2, or isotype control) and cultured with said antibody for the indicated number of days (1, 2, 3, 5, or 6) FIG. 84B is a bar graph showing the level of secreted IL-1β by T cells activated/expanded with the indicated antibody (anti-TCRβV 6-5 v1, anti-TCRβV 6-5 v1. OKT3, SP34-2, or isotype control) and cultured with said antibody for the indicated number of days (1, 2, 3, 5, or 6). FIG. 84C is a bar graph showing the level of secreted IL-4 by T cells activated/expanded with the indicated antibody (anti-TCRβV 6-5 v1, anti-TCRβV 6-5 v1. OKT3, SP34-2, or isotype control) and cultured with said antibody for the indicated number of days (1, 2, 3, 5, or 6). FIG. 84D is a bar graph showing the level of secreted IL-6 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 84E is a bar graph showing the level of secreted IL-10 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 84F is a bar graph showing the level of secreted TNFα by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 84G is a bar graph showing the level of secreted IL-2 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6).

FIG. 85A is a bar graph showing the level of secreted IFNγ by T cells activated/expanded with the indicated antibody (anti-TCRβV 6-5 v1, anti-TCRβV 6-5 v1. OKT3, or SP34-2) and cultured with said antibody for the indicated number of days (1, 2, 3, 4, 5, 6, or 8). FIG. 85B is a bar graph showing the level of secreted IL-1β by T cells activated/expanded with the indicated antibody (anti-TCRβV 6-5 v1, anti-TCRβV 6-5 v1. OKT3, or SP34-2) and cultured with said antibody for the indicated number of days (1, 2, 3, 4, 5, 6, or 8). FIG. 85C is a bar graph showing the level of secreted IL-4 by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 85D is a bar graph showing the level of secreted IL-6 by T cells activated/expanded with the indicated antibody (anti-TCRβV 6-5 v1, anti-TCRβV 6-5 v1. OKT3, or SP34-2) and cultured with said antibody for the indicated number of days (1, 2, 3, 4, 5, 6, or 8). FIG. 85E is a bar graph showing the level of secreted IL-10 by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 85F is a bar graph showing the level of secreted TNFα by T cells activated/expanded with the indicated antibody (anti-TCRβV 6-5 v1, anti-TCRβV 6-5 v1. OKT3, or SP34-2) and cultured with said antibody for the indicated number of days (1, 2, 3, 4, 5, 6, or 8). FIG. 85G is a bar graph showing the level of secreted IL-2 by T cells activated/expanded with the indicated antibody (anti-TCRβV 6-5 v1, anti-TCRβV 6-5 v1. OKT3, or SP34-2) and cultured with said antibody for the indicated number of days (1, 2, 3, 4, 5, 6, or 8).

FIG. 86A is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 7). FIG. 86B is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 8). FIG. 86C is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 7). FIG. 86D is a bar graph showing the level of secreted IL-17A by T cells activated/expanded with the indicated antibody (anti-TCRβV 6-5 v1 or SP34-2) and cultured with said antibody for the indicated number of days (1, 3, 5, or 7).

FIG. 87A is a bar graph showing the level of secreted IFNγ by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87B is a bar graph showing the level of secreted IL-1β by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87C is a bar graph showing the level of secreted IL-4 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87D is a bar graph showing the level of secreted IL-6 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87E is a bar graph showing the level of secreted IL-10 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87F is a bar graph showing the level of secreted TNFα by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87G is a bar graph showing the level of secreted IL-2 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87H is a bar graph showing the level of secreted IL-12p70 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87I is a bar graph showing the level of secreted IL-13 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87J is a bar graph showing the level of secreted IL-8 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87K is a bar graph showing the level of secreted exotoxin by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87L is a bar graph showing the level of secreted exotoxin-3 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87M is a bar graph showing the level of secreted IL-8 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87N is a bar graph showing the level of secreted IP-10 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87O is a bar graph showing the level of secreted MCP-1 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87P is a bar graph showing the level of secreted MCP-4 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87Q is a bar graph showing the level of secreted MDC by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87R is a bar graph showing the level of secreted MIP-1a by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87S is a bar graph showing the level of secreted MIP-1b by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87T is a bar graph showing the level of secreted TARC by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87U is a bar graph showing the level of secreted GMCSF by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87V is a bar graph showing the level of secreted IL-12-23p40 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87W is a bar graph showing the level of secreted IL-15 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87X is a bar graph showing the level of secreted IL-16 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87Y is a bar graph showing the level of secreted IL-17a by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87Z is a bar graph showing the level of secreted IL-1a by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87AA is a bar graph showing the level of secreted IL-5 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87BB is a bar graph showing the level of secreted IL-7 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87CC is a bar graph showing the level of secreted TNF-β by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 87DD is a bar graph showing the level of secreted VEGF by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8).

FIG. 88 shows a graphical representation of the relation of sequences between different TCRVB clonotype subfamilies.

FIG. 89A is a bar graph showing the percentage of cytokine release from PBMCs activated/expanded for eight days using the indicated antibody (anti-TCRβV 12-3/4 vi or SP34-2). FIG. 89B is a bar graph showing the percentage of cytokine release from PBMCs activated/expanded for eight days using the indicated antibody (anti-TCRβV 5 or SP34-2). FIG. 89C is a bar graph showing the percentage of cytokine release from PBMCs activated/expanded for eight days using the indicated antibody (anti-TCRβV 10 or SP34-2).

FIG. 90A a bar graph showing the level of secreted IFNγ by T cells activated/expanded with the indicated antibody for the indicated number of days (3 or 6). FIG. 90B a bar graph showing the level of secreted IL-10 by T cells activated/expanded with the indicated antibody for the indicated number of days (3 or 6). FIG. 90C a bar graph showing the level of secreted IL-17A by T cells activated/expanded with the indicated antibody for the indicated number of days (3 or 6). FIG. 90D a bar graph showing the level of secreted IL-1a by T cells activated/expanded with the indicated antibody for the indicated number of days (3 or 6). FIG. 90E a bar graph showing the level of secreted IL-1β by T cells activated/expanded with the indicated antibody for the indicated number of days (3 or 6). FIG. 90F a bar graph showing the level of secreted IL-6 by T cells activated/expanded with the indicated antibody for the indicated number of days (3 or 6). FIG. 90G a bar graph showing the level of secreted TNFα by T cells activated/expanded with the indicated antibody for the indicated number of days (3 or 6). FIG. 90H a bar graph showing the level of secreted IL-2 by T cells activated/expanded with the indicated antibody for the indicated number of days (3 or 6).

FIG. 91 is a bar graph summarizing data from FACS analysis of PBMCs activated/expanded for 6 days using the indicated anti-TCRVβ antibody.

FIG. 92A a bar graph showing the level of secreted IFNγ by T cells activated/expanded with the indicated antibody for the indicated number of days (1, 3, 5, or 7). FIG. 92B a bar graph showing the level of secreted IL-10 by T cells activated/expanded with the indicated antibody for the indicated number of days (1, 3, 5, or 7). FIG. 92C a bar graph showing the level of secreted IL-17A by T cells activated/expanded with the indicated antibody for the indicated number of days (1, 3, 5, or 7). FIG. 92D a bar graph showing the level of secreted IL-1a by T cells activated/expanded with the indicated antibody for the indicated number of days (1, 3, 5, or 7). FIG. 92E a bar graph showing the level of secreted IL-1β by T cells activated/expanded with the indicated antibody for the indicated number of days (1, 3, 5, or 7). FIG. 92F a bar graph showing the level of secreted IL-6 by T cells activated/expanded with the indicated antibody for the indicated number of days (1, 3, 5, or 7). FIG. 92G a bar graph showing the level of secreted IL-4 by T cells activated/expanded with the indicated antibody for the indicated number of days (1, 3, 5, or 7). FIG. 92H a bar graph showing the level of secreted IL-2 by T cells activated/expanded with the indicated antibody for the indicated number of days (1, 3, 5, or 7).

FIG. 93 is a bar graph summarizing data from FACS analysis of PBMCs activated/expanded for 7 days using the indicated anti-TCRVβ antibody.

FIG. 94A is a bar graph showing the level of secreted IFNγ by T cells activated/expanded with the indicated antibody for the indicated number of days (3 or 6). FIG. 94B a bar graph showing the level of secreted IL-10 by T cells activated/expanded with the indicated antibody for the indicated number of days (3 or 6). FIG. 94C a bar graph showing the level of secreted IL-17A by T cells activated/expanded with the indicated antibody for the indicated number of days (3 or 6). FIG. 94D a bar graph showing the level of secreted IL-1a by T cells activated/expanded with the indicated antibody for the indicated number of days (3 or 6). FIG. 94E a bar graph showing the level of secreted IL-1β by T cells activated/expanded with the indicated antibody for the indicated number of days (3 or 6). FIG. 94F a bar graph showing the level of secreted IL-6 by T cells activated/expanded with the indicated antibody for the indicated number of days (3 or 6). FIG. 94G a bar graph showing the level of secreted IL-4 by T cells activated/expanded with the indicated antibody for the indicated number of days (3 or 6). FIG. 94H a bar graph showing the level of secreted TNFα, by T cells activated/expanded with the indicated antibody for the indicated number of days (3 or 6). FIG. 94I a bar graph showing the level of secreted IL-2 by T cells activated/expanded with the indicated antibody for the indicated number of days (3 or 6).

FIG. 95A is a bar graph showing the level of secreted IFN-γ by T cells activated/expanded with the indicated antibody (anti-TCRβV 6-5 v1 (plate coated), anti-CD3s (plate coated), anti-TCRβV 6-5 v1 (in solution), or anti-CD3ε (in solution) and cultured with said antibody for the indicated number of days (1, 3, 5, or 7). FIG. 95B is a bar graph showing the level of secreted IFN-γ by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95C is a bar graph showing the level of secreted IL-1b by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95D is a bar graph showing the level of secreted IL-6 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95E is a bar graph showing the level of secreted IL-10 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95F is a bar graph showing the level of secreted IL-15 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95G is a bar graph showing the level of secreted IL-17A by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95H is a bar graph showing the level of secreted IL-1a by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95I is a bar graph showing the level of secreted IL-1b by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95J is a bar graph showing the level of secreted IL-2 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95K is a bar graph showing the level of secreted IL-4 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 95L is a bar graph showing the level of secreted TNF-α by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7).

FIG. 96 is a FACS plot showing the showing the ability of MH3-2 to bind PBMCs from one of two donors when the PBMCs are either preincubated with TM23 or not (MH3-2 Alone).

FIG. 97 is a FACS plot showing the ability of MH3-2 to bind PBMCs from one of two donors when the PBMCs are either preincubated with TM23 or not (MH3-2 Alone).

FIG. 98A is a bar graph showing the polyfunctional strength index (PSI) of PBMC CD4+ T cells. CD4+ T cells expanded with anti-CD3 antibody, (CD3 Expanded T cells), and CD4+ T cells expanded with anti-TCRVβ 6-5 antibody (Drug Expanded T cells). The Effector mediators are Granzyme B, IFNγ, MIP-1α, perforin, TNFα, and TNFβ. The Stimulatory mediators are IL-5. The Chemoattractive mediators are MIP-1b. FIG. 98B is a bar graph showing the polyfunctional strength index (PSI) of PBMC CD8+ T cells, CD8+ T cells expanded with anti-CD3 antibody, (CD3 Expanded T cells), and CD8+ T cells expanded with anti-TCRVβ 6-5 antibody (Drug Expanded T cells) The Effector mediators are Granzyme B, IFNγ, MIP-1α, perforin, and TNFβ. The Chemoattractive mediators are MIP-1b and RANTES.

FIG. 99 is a schematic of the experimental design for the pharmacokinetic (PK) profile and dosing strategy of the multifunctional polypeptide molecule as described herein.

FIG. 100 shows Table 9, which depicts alignment of TCRβV amino acid sequences (SEQ ID NOS 3457-3516, 3669-3673, 3522, 3674-3675, 3525, 3676-3687, 3538, 3688-3698 and 3550-3639, respectively, in order of appearance). The alignment of TCRβV amino acid sequences in Table 9 underscores the diversity of TCR sequences. In particular, the TRBV sequences from different subfamilies are considerably different from each other.

FIG. 101 shows alignment of affinity matured humanized Antibody A-H VL sequences (SEQ ID NOS: 3377-3389, respectively, in order of appearance).

FIG. 102 shows alignment of affinity matured humanized Antibody A-H VH sequences (SEQ ID NOS: 3390-3436, respectively, in order of appearance).

FIG. 103A shows an exemplary embodiment (e.g., BKM0186) of multifunctional molecules comprising a TCRβV-binding moiety and a cytokine polypeptide (e.g., IL2 or IL2-C125A) as described herein. FIG. 103B shows an exemplary embodiment of multifunctional molecules comprising a TCRβV-binding moiety and a cytokine polypeptide as described herein. FIGS. 103C, 103D, 103E, and 103F show exemplary embodiments of multifunctional molecules comprising a first TCRβV-binding moiety, a second TCRβV-binding moiety, and two cytokine polypeptides as described herein. In some embodiments, the cytokine polypeptide comprises IL-2 or a functional fragment or a functional variant thereof, IL2-C125A or a functional fragment or a functional variant thereof, IL-15 or a functional fragment or a functional variant thereof, IL-7 or a functional fragment or a functional variant thereof, IL-12 or a functional fragment or a functional variant thereof, or IL-21 or a functional fragment or a functional variant thereof. In embodiments, the cytokine polypeptide further comprises a cytokine receptor. In some embodiments, the cytokine polypeptide comprises IL-15 linked to a IL-15Ra. In some embodiments, the cytokine polypeptide comprises IL-15 linked to a IL-15Ra sushi domain. In some embodiments, the cytokine polypeptide comprises a cytokine dimer. In some embodiments, the cytokine polypeptide comprises an IL-12 beta subunit linked to an IL-12 alpha subunit.

FIG. 104 shows FACS plots showing binding of BKM0186 to different immune cell populations in Human PBMCs.

FIG. 105 shows binding of BKM0186 to pure human T cells expressing either Vβ6 or CD25 (IL-2Rα) or both.

FIG. 106 shows in vitro concentration-effect relationships for BKM0186-mediated in vitro expansion of Vβ6 T cells and activated (CD25) Vβ6 T cells from human PBMCs at day 5 as a % of total T-Cytotoxic (CD8) and T-helper (CD4) populations. Left graph: T-cytotoxic lymphocytes: right graph: T-helper lymphocytes.

FIG. 107 shows in vitro TCR sequencing. PBMCs were incubated with 100 nM of BKM0186 for 5 days and T cells were sequenced for TCR β chain V (TRBV) genes. Compared to unstimulated T cells (grey), BKM0186 selectively expanded T cells bearing TRBV6-1, TRBV6-2, TRBV6-3, TRBV6-5, and TRBV10-3.

FIG. 108A and FIG. 108B show a series of graphs (FIG. 108A) and a series of FACS plots (FIG. 108B) exhibiting activation of CD4+ and CD8+ T cells as assessed by CD25 expression following stimulation with BKM0186. RSV-IL2 and Anti-TCRVβ6 control in solution.

FIG. 109 shows a series of FACS plots demonstrating differentiation of memory T cells mediated by BKM0186 in comparison to unstimulated and the controls RSV-IL2 and anti-TCRVβ6. Upper left quadrant represents Central memory (CM), lower left quadrant represents Effector memory (EM), upper right quadrant represents Naïve (N) and lower right quadrant represents Effector memory RA (TEMRA).

FIG. 110 shows in vitro concentration-effect relationships for BKM0186-induced cytokine release from human PBMCs at day 4 using MSD V-plex human cytokine panel.

FIG. 111 shows BKM0186-mediated killing of human tumor organoids generated from primary, patient-derived tissue from colorectal and NSCLC cancer patients. Vertical bars represent percentage of organoid area reduced relative to isotype control following incubation of organoids with BKM0186 and autologous TILs.

FIG. 112 shows tumor growth curves of mBKM0186-treated EMT6 tumor-bearing mice. Studies were performed in randomized mice with tumor volumes of 80-150 mm³. For all models except MC38, mice were dosed for 3 weeks with a weekly dosing of 0.5-1.5 mg/kg and survival was determined based on 2000 mm³ tumor volume end point.

FIG. 113 shows tumor growth curves of mBKM0186-treated mice. Studies were performed in randomized mice with tumor volumes of 80-150 mm³. For all models except MC38, mice were dosed for 4 weeks with a weekly dosing of 1-1.5 mg/kg and survival was determined based on 2000 mm³ tumor volume end point. For MC38, mice were given first dose of 3 mg/kg followed by 1 mg/kg for subsequent three weekly (QW) doses.

FIG. 114 shows Kaplan-Meier survival curves of treated mice. Studies were performed in randomized mice with tumor volumes of 80-150 mm³. For all models except MC38, mice were dosed for 4 weeks with a weekly dosing of 1-1.5 mg/kg and survival was determined based on 2000 mm³ tumor volume end point. For MC38, mice were given first dose of 3 mg/kg followed by 1 mg/kg for subsequent three weekly (QW) doses.

FIG. 115 shows the experimental design for the tumor rechallenge study. Cured EMT6 tumor bearing mice were rechallenged with EMT6 tumor cells in one flank and CT26 tumor cells in another flank and monitored for tumor growth for 28 days.

FIG. 116 shows the results of the tumor rechallenge study. While the EMT6 tumors were rejected. CT26 tumors grew, suggesting that the memory response against EMT6 tumors likely mediated through mBKM0186 treatment has been established.

FIG. 117 shows immune profiling of T cells in blood and tumor tissue on day 14 post dosing of mBKM0186.

FIG. 118 shows tumor growth curves of EMT6 tumors after weekly (QW) treatment of mice bearing 150 mm³ tumors with 1 mg/kg of mBKM0186 with and without depletion of Vβ-specific T cells Filled Triangles indicate dosing intervals of the depleting antibodies and open Triangles indicate dosing intervals of mBKM0186.

FIG. 119A and FIG. 119B show Pharmacokinetic profiles of BKM0186 (FIG. 119A) and BKM0281 (FIG. 119B) administered single dose IV in cynomolgus monkeys.

FIG. 120A shows T cell expansion following a single IV dose of BKM0186. FIG. 120B shows T cell expansion following a single IV dose BKM0281. n=3 Monkeys, n=1 monkey vehicle control.

FIG. 121 shows serum soluble CD25 levels in monkeys administered a single IV dose of BKM0186. Mean values, n=2-3 monkeys per group.

FIG. 122A shows serum IL-6 levels in monkeys administered a single IV dose of BKM0186. FIG. 122B shows serum IL-6 levels in monkeys administered a single IV dose of BKM0281. Mean values, n=2-3 monkeys per group.

FIG. 123A shows serum IFN-γ levels in monkeys administered a single IV dose of BKM0186. FIG. 123B shows IFN-γ levels in monkeys administered a single IV dose of BKM0281. Mean values, n=2-3 monkeys per group. Mean values, n=2-3 monkeys per group.

FIG. 124 shows in vitro concentration-effect relationships for bispecific-mediated in vitro expansion of Vβ6 T cells.

FIGS. 125A and 125B show exemplary embodiments of multifunctional molecules comprising a TCRβV-binding moiety and a cytokine molecule (e.g., wild-type human IL2 or IL 15-IL 15R sushi fusion) as described herein. FIGS. 125C-125T show exemplary embodiments of multifunctional molecules, e.g., multifunctional molecules comprising a tumor-associated antigen (TAA) binding moiety, a TCRβV-binding moiety, and at least one cytokine molecule or a functional fragment or functional variant thereof as described herein. In some embodiments, a tumor-associated antigen (TAA)-binding moiety and/or a TCRβV-binding moiety are antibody, an antigen binding fragment thereof or an antibody fragment. In some embodiments, an antigen binding fragment or an antibody fragment comprises Fab. Fab′, F(ab′)₂, F(ab)₂, variable fragment (Fv), domain antibody (dAb), or single chain variable fragment (scFv). In some embodiments, the at least one cytokine molecule or a functional fragment or functional variant thereof is selected from the group consisting of interleukin-2 (IL-2) or a functional fragment or functional variant thereof, interleukin-7 (IL-7) or a functional fragment or functional variant thereof, interleukin-12 (IL-12) or a functional fragment or functional variant thereof, interleukin-15 (IL-15) or a functional fragment or functional variant thereof, interleukin-18 (IL-18) or a functional fragment or functional variant thereof, interleukin-21 (IL-21) or a functional fragment or functional variant thereof, or interferon gamma or a functional fragment or functional variant thereof, or a combination thereof. In some embodiments, exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein comprises a dimerization module comprising an Fc region comprising N297A mutation. FIGS. 125C-125H show exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the multifunctional molecules comprise an exemplary dimerization module, e.g., an Fc region comprising Knob-in-hole mutations and disulfide bridges. FIGS. 1251-125N show exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the multifunctional molecules comprise an exemplary dimerization module, e.g., an Fc region comprising disulfide bridges, but not comprising Knob-in-hole mutations. FIGS. 1250-125T show exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the multifunctional molecules comprise an exemplary dimerization module, e.g., an Fc region not comprising disulfide bridges nor Knob-in-hole mutations. FIGS. 125C-125E, 125I-125K, and 1250-135Q show exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the TCRβV-binding moiety comprises an scFv. FIGS. 125F-125H, 125L-125N, and 125R-135T show exemplary embodiments of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the TCRβV-binding moiety comprises an Fab. FIG. 125U shows an exemplary embodiment of multifunctional molecules, e.g., multifunctional molecules comprising a tumor-associated antigen (TAA)-binding moiety, a TCRβV-binding moiety, and at least one cytokine molecule or a functional fragment or functional variant thereof as described herein, wherein the multifunctional molecule comprise two TAA-binding moieties. In some embodiments, the at least one cytokine molecule or a functional fragment or functional variant thereof is interleukin-7 (IL-7) or a functional fragment or functional variant thereof, interleukin-12 (IL-12) or a functional fragment or functional variant thereof, interleukin-15 (IL-15) or a functional fragment or functional variant thereof, interleukin-18 (IL-18) or a functional fragment or functional variant thereof, interleukin-21 (IL-21) or a functional fragment or functional variant thereof, or interferon gamma or a functional fragment or functional variant thereof, or a combination thereof instead of interleukin-2 (IL-2) or a functional fragment or functional variant thereof as indicated as “IL2”.

FIGS. 126A, 126B and 126C show that anti-CD20 TriSTAR molecules, which are exemplary embodiments of the multifunctional molecule as provided herein, induce highly potent Vβ-specific expansion, CD8+ T cell activation and killing of B cells in vitro. FIG. 126A depicts in vitro murine Vβ T cell expansion, FIG. 126B depicts in vitro murine CD8+ T cell activation and expansion, and FIG. 126C depicts in vitro murine B cell depletion.

FIG. 127 shows that anti-CD20 TriSTAR T-cell engagers (TCEs), which are exemplary embodiments of the multifunctional molecule as provided herein, also show potent B cell-depletion in vivo upon single IP dose of 3 mg/kg. In the same study anti-CD3/CD20 bispecific was also tested and showed to have comparable potency in vivo.

FIGS. 128A and 128B show in vitro expansion with anti-MSLN TriSTAR (BLM0155), which is an exemplary embodiment of the multifunctional molecule as provided herein, and CD3 TCE (BMM0388). Both TriSTAR and CD3 engagers expanded in the presence of plate-bound MSLN. FIG. 128A depicts % CD25⁺, and FIG. 128B depicts EC50 and Emax (%).

FIGS. 129A and 129B show in vitro expansion with anti-MSLN Vb bispecific TCEs (BLM0158, 1×1 format: BMM0274, 2×2 format), which are exemplary embodiments of the multifunctional molecule as provided herein, and CD3 TCE. Both Vb and CD3 engagers expanded in the presence of plate-bound MSLN. CD3 TCE expanded much more. FIG. 129A depicts % CD25⁺, and FIG. 129B depicts EC50 and Emax (%).

FIGS. 130A and 130B show in vitro cytotoxicity with anti-MSLN TriSTARs (BLM0155, 1×1×1 TriSTAR: BMM0462, 2×1×1 TriSTAR format), which are exemplary embodiments of the multifunctional molecule as provided herein, and CD3 TCE (BMM0388). FIG. 130A depicts % cytotoxicity, and FIG. 130B depicts EC50 and Emax (%).

FIGS. 131A and 131B show in vitro cytotoxicity with anti-MSLN Vb bispecific TCEs (BLM0158, 1×1 format: BMM0274, 2×2 format), which are exemplary embodiments of the multifunctional molecule as provided herein, and CD3 TCE. FIG. 131A depicts % cytotoxicity, and FIG. 131B depicts EC50 and Emax (%).

FIGS. 132A and 132B show in vitro expansion with anti-MSLN TriSTAR murine surrogates (BMM0449, 1×1×1 TriSTAR format: BMM0456, 2×1×1 TriSTAR format), which are exemplary embodiments of the multifunctional molecule as provided herein, and CD3 TCE (BMM0456, 1×1 CD3 TCE; BMM0469, 2×1 CD3 TCE). All the TCE constructs showed potent expansion in the presence of the plate-bound MSLN. FIG. 132A depicts % CD25′, and FIG. 132B depicts EC50 and Emax (%).

FIGS. 133A-133D show in vitro expansion with anti-MSLN Vb TCE murine surrogates (BMM0446, 1×1 format: BMM0439, 2×2 format), which is an exemplary embodiment of the multifunctional molecule as provided herein, and CD3 TCE (BMM0456). All the constructs demonstrated expansion in the presence of plate-bund MSLN. FIGS. 133A and 133C depict % CD25⁺, and FIGS. 133B and 133D depict EC50 and Emax (%).

FIG. 134 shows in vitro cytotoxicity of EMT6 cells with anti-MSLN TriSTAR murine surrogate (BMM0449) and CD3 TCE (BMM0456).

FIGS. 135A and 135B show in vitro cytotoxicity of EMT6 cells anti-MSLN Vb TCE murine surrogates (BMM0446, 1×1 format: BMM0439, 2×2 format), which are exemplary embodiments of the multifunctional molecule as provided herein, and CD3 TCE FIG. 135A depicts % cytotoxicity, and FIG. 135B depicts EC50 and Emax (%).

FIGS. 136A and 136B show that anti-MSLN TriSTAR murine surrogate (BMM0449), which are exemplary embodiments of the multifunctional molecule as provided herein, show potent anti-tumor activity vs, αCD3 TCE (BMM0456) in EMT6 syngeneic mouse model. Tumor bearing mice received weekly doses of 3 mg/kg of the test articles for 4 weeks. FIG. 136A depicts a scheme of the experimental design, and FIG. 136B depicts mean tumor volume.

FIG. 137 shows PK data for anti-MSLN TriSTAR murine surrogate (BMM0449), which is an exemplary embodiments of the multifunctional molecule as provided herein.

FIG. 138 shows that anti-gp75 Vβ bispecific TCEs murine surrogates (BNM0104, 1×1 format and BNM0869, 2×2 format), which are exemplary embodiments of the multifunctional molecule as provided herein, showed limited activity in solid (B16F10) syngeneic tumors. Limited anti-tumor activity of 1×1 and 2×2 Vβ×gp75 TCEs, which are exemplary embodiments of the multifunctional molecule as provided herein, in B16F10 solid tumor model.

FIG. 139 shows that when combined with an exemplary multifunctional molecule comprising a anti-Vβ binder and IL-2 (BKM0307), anti-gp75 Vβ murine surrogate TCEs (BNM0104, 1×1 format and BNM0869, 2×2 format), which are exemplary embodiments of the multifunctional molecule as provided herein, are highly active. 1×1 and 2×2 Vβ×gp75 T cell engagers are active in B16F10 tumor model when dosed with or following an exemplary multifunctional molecule comprising a anti-Vβ binder and IL-2.

FIG. 140 shows that anti-gp75 TriSTAR TCE murine surrogate (BNM0094), which is an exemplary embodiments of the multifunctional molecule as provided herein, promotes superior anti-tumor activity vs. anti-gp75/αCD3 TCEs (BNM0092) in B16F10 model. Significant anti-tumor activity in refractory B16 mouse with TriSTAR TCE was observed.

FIGS. 141A-141C show pharmacokinetics of TriSTAR construct (BNM0094), which is an exemplary embodiments of the multifunctional molecule as provided herein. FIG. 141A depicts the results of an exemplary embodiments of the multifunctional molecule as provided herein, BNM0094, and FIG. 141B depicts the results of a CD3×gp75 bispecific molecule, BNM0092. FIG. 141C depicts the experimental design.

FIGS. 142A-142C show that in B16F10 tumors. TriSTAR, which is an exemplary embodiments of the multifunctional molecule as provided herein, promotes greater expansion of cytotoxic CD8 T cells in the tumor microenvironment. FIG. 142A depicts % CD8 of CD45 cells, FIG. 142B depicts % CD& CD25 cells, and FIG. 142C depicts % CD8 granzyme B cells.

FIGS. 143A and 143B show Vβ13+ T cell expansion by Tri-STAR, which is an exemplary embodiments of the multifunctional molecule as provided herein, in tumor microenvironment (TME). FIG. 143A depicts CD8 Vb cells/mg tumor, and FIG. 143B depicts % CD8 Vb subset frequency.

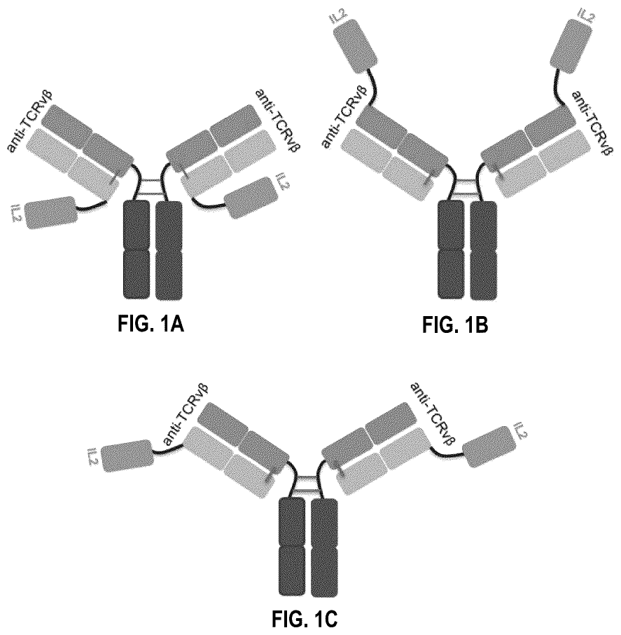

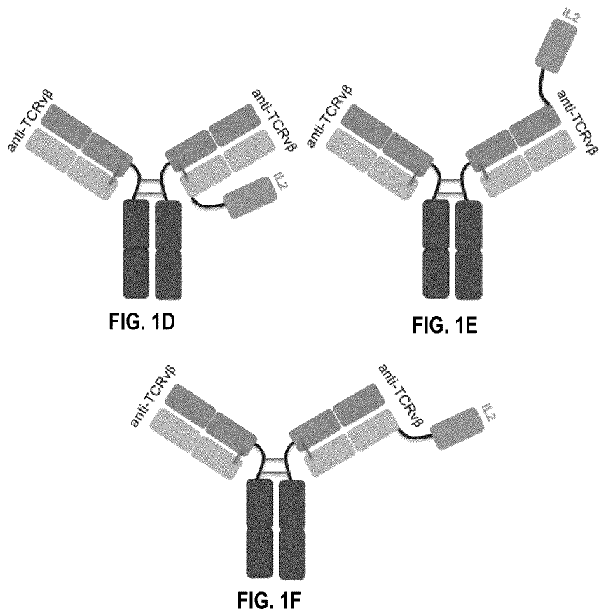

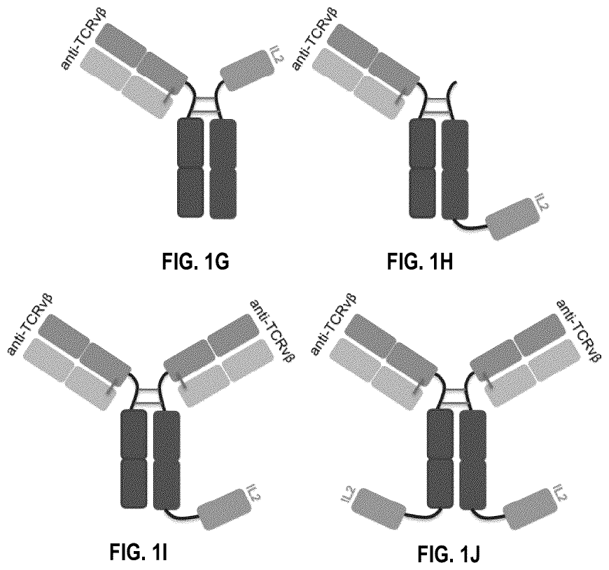

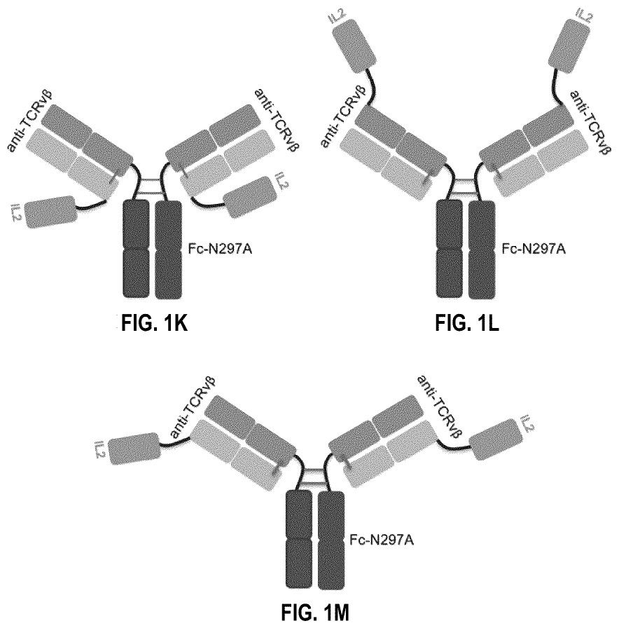

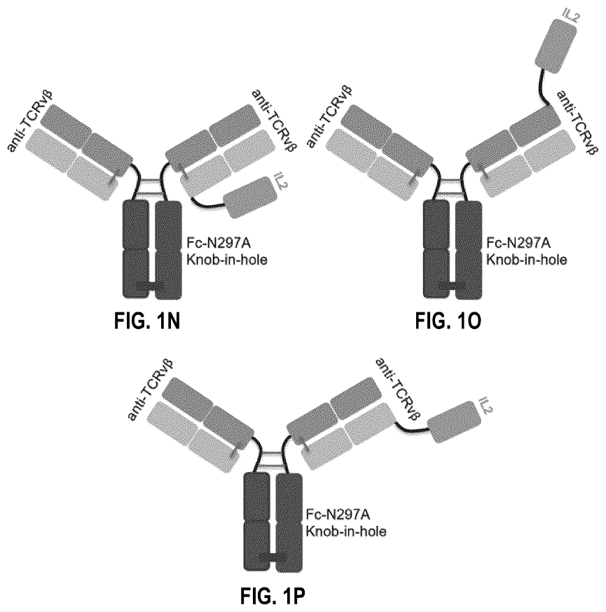

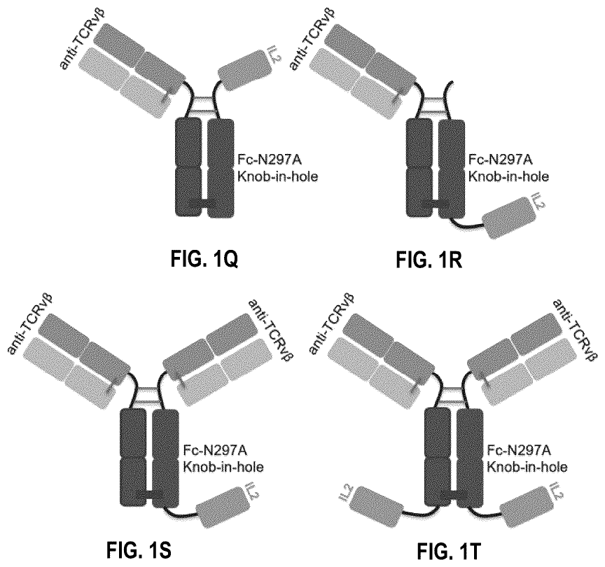

### DETAILED DESCRIPTION

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g. Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat. E. A., et al. (1991) Sequences of Proteins of Immunological Interest. Fifth Edition, U.S. Department of Health and Human Services, NIH Publication No. 91-3242, and Chothia, C et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system. e.g., to protect against agents of infection and foreign matter. In embodiments, this term includes leukocytes. e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid.” “nucleic acid sequence.” “nucleotide sequence,” or “polynucleotide sequence.” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRβV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score =50, wordlength=3 to obtain amino acid sequences homologous to protein molecules of the invention. To obtain gapped alignments for comparison purposes. Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids: analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

“Interleukin-2” also known as IL2, IL-2, IL 2, TCGF, lymphokine, and interleukin 2, as referred to herein, includes any of the recombinant or naturally-occurring forms of IL-2 or variants or homologs thereof that have or maintain IL-2 activity (e.g., at least 40% 50%, 60%, 70%, 80%, 90%, 95%, 96%, 97%, 98%, 99% or 100% activity) In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring IL-2. In some embodiments, IL-2 is substantially identical to the protein identified by the UniProt reference number P60568 or a variant or homolog having substantial identity thereto.

### Anti-TCRβV Antibodies

### Human T Cell Receptor (TCR) Complex

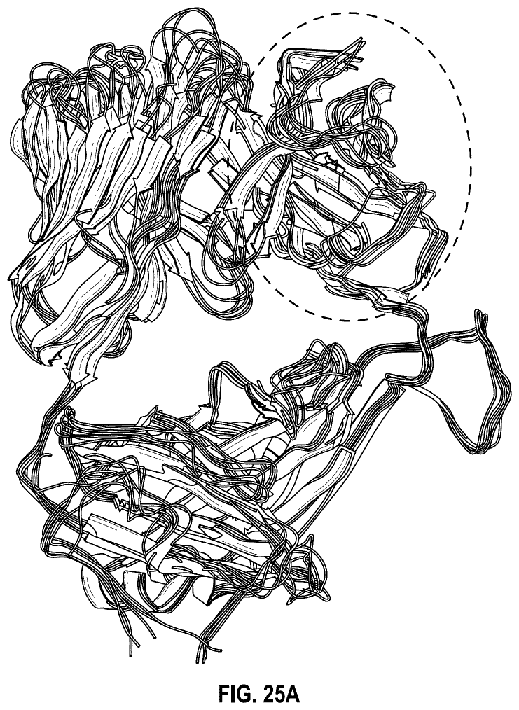

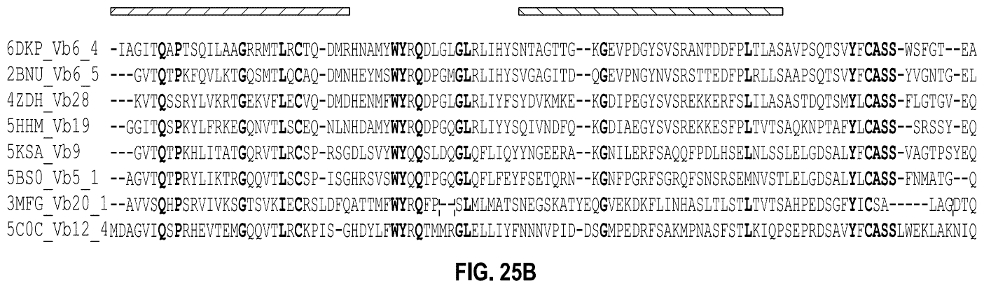

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRβV chains can be further classified into 30 subfamilies (TRBV1-30) Despite their high structural and functional homology, the amino acid sequence homology in the TRBV genes is very low. Only 4 amino acids out of approximately 95 are identical while 10 additional amino acids are conserved among all subfamilies (see, an alignment of TCRβV amino acid sequences in Table 9). Nevertheless, TCRs formed between alpha and beta chains of highly diverse sequences show a remarkable structural homology (FIGS. 25A and 25B) and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G, and Nyland J. (2010) Chapter 10: Major Histocompatibility Complex and T-cell Receptors-Role in Immune Responses. In: Microbiology and Immunology on-line. University of South Carolina School of Medicine). The TCR alpha chain consists of V. J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01, or a variant thereof, e.g., a variant having 85%, 90%, 95%, 99% or more identity the naturally-occurring sequence. TCRβ V6-5*01 is also known as TRBV65; TCRβV6S5; TCRβV13S1, or TCRβ V13.1. The amino acid sequence of TCRβ V6-5*01, e.g., human TCRβ V6-5*01, is known in that art, e.g., as provided by IMGT ID L36092. In some embodiments. TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAG
GTCCAGTGAATGCTGGTGTCACTCAGACCCCAAAATTCCAGGTCCTGAA
GACAGGACAGAGCATGACACTGCAGTGTGCCCAGGATATGAACCATGAA
TACATGTCCTGGTATCGACAAGACCCAGGCATGGGGCTGAGGCTGATTC
ATTACTCAGTTGGTGCTGGTATCACTGACCAAGGAGAAGTCCCCAATGG
CTACAATGTCTCCAGATCAACCACAGAGGATTTCCCGCTCAGGCTGCTG
TCGGCTGCTCCCTCCCAGACATCTGTGTACTTCTGTGCCAGCAGTTACT
C
SEQ ID NO: 44
MSIGLLCCAALSLLWAGPVNAGVTQTPKFQVLKTGQSMTLQCAQDMNHE
YMSWYRQDPGMGLRLIHYSVGAGITDQGEVPNGYNVSRSTTEDFPLRLL
SAAPSQTSVYFCASSY

### TCR Beta V (TCRβV)

Diversity in the immune system enables protection against a huge array of pathogens. Since the germline genome is limited in size, diversity is achieved not only by the process of V (D) J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7) pp: 493-502; Wei S, and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRVB, TRBV, TCRβV, TCRVβ or TRβV are used interchangeably herein and refer to a TCR beta V chain, e.g., as described herein.

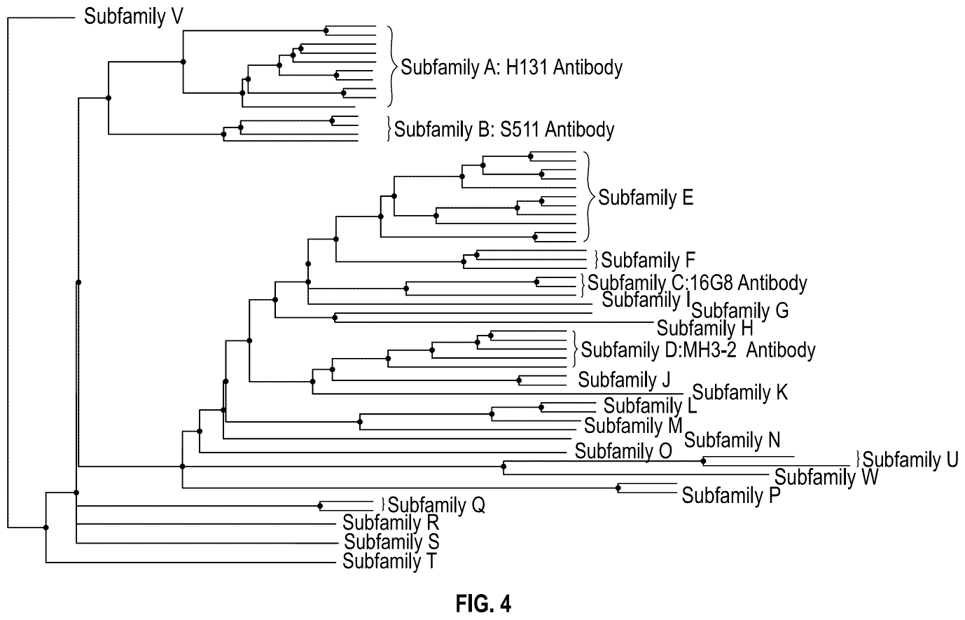

In some embodiments, provided herein is an anti-TCRβV antibody molecule that binds to human TCRβV, e.g., a TCRβV family, e.g., gene family or a variant thereof. In some embodiments a TCRβV gene family comprises one or more subfamilies, e.g., as described herein, e.g., in FIG. 4, Table 8A or Table 8B. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments. TCRβ V6 comprises TCRβ V6-4*02, or a variant thereof. In some embodiments, TCRβ V6 comprises TCRβ V6-9*01, or a variant thereof. In some embodiments. TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments. TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments. TCRβ V6, e.g., TCRβ V6-5*01, is recognized, e.g., bound, by SEQ ID NO: 1 and/or SEQ ID NO: 2. In some embodiments. TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof. In some embodiments, the TCRβ V14 subfamily comprises TCRβ V14*01, or a variant thereof. In some embodiments, the TCRβ V16 subfamily comprises TCRβ V16*01, or a variant thereof. In some embodiments, the TCRβ V18 subfamily comprises TCRβ V18*01, or a variant thereof. In some embodiments, the TCRβ V9 subfamily comprises TCRβ V9*01 or TCRβ V9*02, or a variant thereof. In some embodiments, the TCRβ V13 subfamily comprises TCRβ V13*01, or a variant thereof. In some embodiments, the TCRβ V4 subfamily comprises TCRβ V4-2*01, TCRβ V4-3*01, or TCRβ V4-1*01, or a variant thereof. In some embodiments, the TCRβ V3 subfamily comprises TCRβ V3-1*01, or a variant thereof. In some embodiments, the TCRβ V2 subfamily comprises TCRβ V2*01, or a variant thereof. In some embodiments, the TCRβ VIS subfamily comprises TCRβ V15*01, or a variant thereof. In some embodiments, the TCRβ V30 subfamily comprises TCRβ V30*01, or TCRβ V30*02, or a variant thereof. In some embodiments, the TCRβ V19 subfamily comprises TCRβ V19*01, or TCRβ V19*02, or a variant thereof. In some embodiments, the TCRβ V27 subfamily comprises TCRβ V27*01, or a variant thereof. In some embodiments, the TCRβ V28 subfamily comprises TCRβ V28*01, or a variant thereof. In some embodiments, the TCRβ V24 subfamily comprises TCRβ V24-1*01, or a variant thereof. In some embodiments, the TCRβ V20 subfamily comprises TCRβ V20-1*01, or TCRβ V20-1*02, or a variant thereof. In some embodiments, the TCRβ V25 subfamily comprises TCRβ V25-1*01, or a variant thereof. In some embodiments, the TCRβ V29 subfamily comprises TCRβ V29-1*01, or a variant thereof.

Exemplary amino acid sequences for TCRβV subfamily members can be found on the ImMunoGene Tics Information System website: http://www.imgt.org/, or in a similar resource.

### Anti-TCRβV Antibodies

Current bispecific constructs designed to redirect T cells to promote tumor cell lysis for cancer immunotherapy typically utilize antibody fragments (Fab, scFv. VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein (as denoted by the circled area in FIG. 25A) and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein, e.g., as denoted by the circled area in FIG. 25A. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. As shown in Table 9, TCRβV proteins from the different TCRβV subfamilies share minimal sequence similarity. However, as shown in FIG. 25A-25B, TCRβV proteins which have minimal sequence similarity, share a similar 3D conformation and structure.

The alignment of TCRβV amino acid sequences in Table 9 underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV: TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as de-scribed in Viney et al., (Hybridoma. 1992 December: 11 (6): 701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV 19, TRBV20-1, TRBV24-1, TRBV25-1, TRBV27, TRBV28, TRBV29-1 and TRBV30. In some embodiments, the anti-TCRβV antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV2, anti-TRBV3-1, anti-TRBV4-1, anti-TRBV4-2, anti-TRBV4-3, anti-TRBV5-1, anti-TRBV5-4, anti-TRBV5-5, anti-TRBV5-6, anti-TRBV5-8, anti-TRBV6-1, anti-TRBV6-2, anti-TRBV6-3, anti-TRBV6-4, anti-TRBV6-5, anti-TRBV6-6, anti-TRBV6-8, anti-TRBV6-9, anti-TRBV7-2, anti-TRBV7-3, anti-TRBV7-4, anti-TRBV7-6, anti-TRBV7-7, anti-TRBV7-8, anti-TRBV7-9, anti-TRBV9, anti-TRBV10-1, anti-TRBV10-2, anti-TRBV 10-3, anti-TRBV11-1, anti-TRBV11-2, anti-TRBV11-3, anti-TRBV 12-3, anti-TRBV 12-4, anti-TRBV12-5, anti-TRBV13, anti-TRBV14, anti-TRBV15, anti-TRBV16, anti-TRBV18, anti-TRBV19, anti-TRBV20-1, anti-TRBV24-1, anti-TRBV25-1, anti-TRBV27, anti-TRBV28, anti-TRBV29-1, or anti-TRBV30. Exemplary anti-TCRβV antibody molecules and the corresponding TCRβV subfamilies recognized by said anti-TCRβV antibody molecules are disclosed in Table 10A.

In some embodiments, the anti-TCRβV antibody molecule binds specifically to TRBV2, TRBV3-1. TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1 or TRBV30. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the anti-TCRβV antibody molecule does not bind to TCRβ V12, or binds to TCRβ V12 with an affinity and/or binding specificity that is less than (e.g., less than about 10%, 20%, 30%. 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5- or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule does not comprise the CDRs of the Antibody B murine antibody.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as de-scribed in U.S. Pat. No. 5,861,155.

In some embodiments, the anti-TCRβV antibody molecule binds to a TCRβV region other than TCRB V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule does not comprise the CDRs of the TM23 murine antibody.

In some embodiments, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence: (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

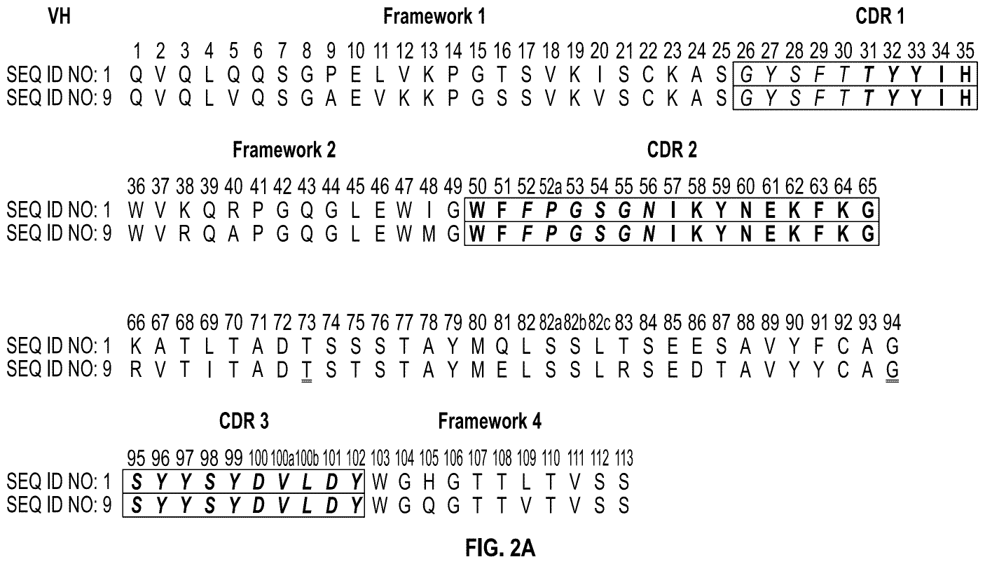

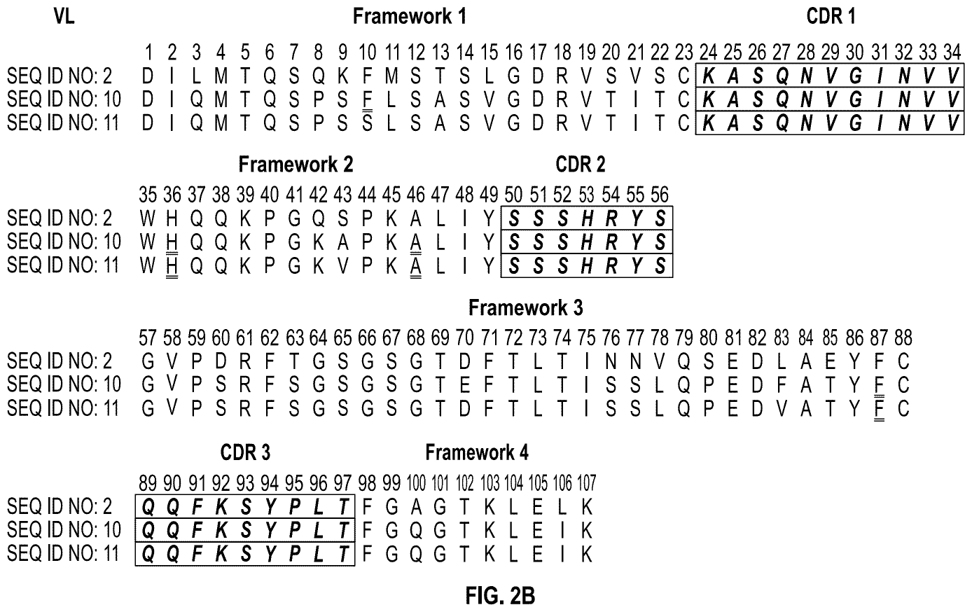

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 2A, or in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in FIG. 2B, or in SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, includes one, two, three, or four heavy chain framework regions shown in FIG. 2A, or a sequence substantially identical thereto.

In some embodiments, the anti-TCRβV antibody molecule, e.g., anti-TCRβ V6 (e.g., anti-TCRβ V6-5*01) antibody molecule, includes one, two, three, or four light chain framework regions shown in FIG. 2B, or a sequence substantially identical thereto.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2, e.g., as shown in FIG. 2B.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2, e.g., as shown in FIG. 2B.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2, e.g., as shown in FIG. 2B.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2, e.g., as shown in FIG. 2B.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenylalanine at position 87, e.g., a substitution at position 87 according to Kabat numbering, e.g., a Tyrosine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering, e.g., a Serine to Phenylalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering. e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering. e.g., a Tyrosine to Phenylalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2, e.g., as shown in FIG. 2A. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2, e.g., as shown in FIG. 2A. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2, e.g., as shown in FIG. 2A. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2, e.g., as shown in FIG. 2A.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO. 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9, or as shown in FIGS. 2A and 2B. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10, or as shown in FIGS. 2A and 2B. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11, or as shown in FIGS. 2A and 2B. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO. 9; and the light chain framework regions 1-4 of A-H.1, e.g., SEQ ID NO: 10, or as shown in FIGS. 2A and 2B. In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11, or as shown in FIGS. 2A and 2B.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 1, or encoded by the nucleotide sequence in Table 1; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO. 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO. 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y). 298 (S to T). 300 (T to E). 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 110, or a sequence having at least 85%, 90%, 95%, 96%, 97%, 98%, 99%, 99.5%, 99.9%, or 100% sequence identity thereto.

Additional exemplary humanized anti-TCRβ V6 antibodies are provided in Table 1. In some embodiments, the anti-TCRβ V6 is antibody A, e.g., humanized antibody A (antibody A-H), as provided in Table 1. In some embodiments, the anti-TCRβV antibody comprises one or more (e.g., all three) of a LC CDR1, LC CDR2, and LC CDR3 provided in Table 1; and/or one or more (e.g., all three) of a HC CDR1, HC CDR2, and HC CDR3 provided in Table 1, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21. A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.41, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21. A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 10A.

The various TCRβV subfamilies and/or subfamily members can be expressed at different levels in individuals, e.g., healthy individuals, as disclosed in Kitaura K. et al (2016), BMC Immunology vol 17:38, the entire contents of which are hereby incorporated by reference. For example, TCRβ V6-5 is represented in approximately 3-6% healthy donors.

The representation of various TCRβV subfamilies and/or subfamily members can also be different in cancer cells. For example, TCRβV is present in about 3-6% of tumor infiltrating T cells irrespective of tumor type (see Li B, et al., Nature Genetics, 2016, vol: 48 (7): 725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising. TCRβ V6-4*01, TCRβ V6-4*02, TCRβ V6-9*01, TCRβ V6-8*01. TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments. TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments. TCRβ V6 comprises TCRβ V6-5*01, or a variant thereof. In some embodiments, TCRβ V6 comprises TCRβ V6-6*02, or a variant thereof. In some embodiments. TCRβ V6 comprises TCRβ V6-6*01, or a variant thereof. In some embodiments, TCRβ V6 comprises TCRβ V6-2*01, or a variant thereof. In some embodiments. TCRβ V6 comprises TCRβ V6-3*01, or a variant thereof. In some embodiments. TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or an amino acid sequence having 85%, 90%, 95%. 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule. e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

-Consensus VH
SEQ ID NO: 231
QVQLVQSGAEVKKPGSSVKVSCKASGH/T/G/YD/T/SFH/R/D/K/
TL/D/K/T/NW/F/T/I/Y/GYIHWVRQAPGQGLEWMGR/WV/I/FF/
S/YA/PGSGN/ST/V/Y/IK/RYNEKFKGRVTITADTSTSTAYMELSSL
RSEDTAVYYCAG/VSY/IYSY/AD/GVLDYWGQGTTVTVSS
-Consensus VH
SEQ ID NO: 3290
QVQLVQSGAEVKKPGSSVKVSCKASGX₁X₂FX₃X₄X₅YIHWVRQAPGQGLE
WMGX₆X₇X₈X₉GSGX₁₀X₁₁X₁₂YNEKFKGRVTITADTSTSTAYMELSSLRSE
DTAVYYCAX₁₃SX₁₄YSX₁₅X₁₆VLDYWGQGTTVTVSS

where-in: X1 is H or Tor G or Y: X2 is D or Tor S; X3 is H or R or D or K or T: X4 is L or D or K or Tor N; X5 is W or F or T or I or Y or G; X6 is R or W: X7 is V or I or F; X8 is F or S or Y; X9 is A or P; X10 is Nor S: X11 is Tor V or Y or I: X12 is K or R; X13 is G or V: X14 is Y or I: X15 is Y or A; and X16 is D or G.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

-Consensus VL
SEQ ID NO: 230
DIQMTQSPSFLSASVGDRVTITCKASQNVG/E/A/DN/DR/KVAWY/HQ
QKPGKAPKALIYSSSHRYK/SGVPSRFSGSGSGTEFTLTISSLQPEDFA
TYFCQQFKSYPLTFGQGTKLEIK

SEQ ID NO: 3289-Consensus VL DIQMTQSPSFLSASVGDRVTITCKASQNVX₁X₂X₃VAWX₄QQKPGKAPKALIYSSSHRYX₅GVPSRFSGS GSGTEFTLTISSLQPEDFATYFCQQFKSYPLTFGQGTKLEIK, wherein X1 is G, E, A or D; X2 is N or D; X3 is R or K: X4 is Y or H; and X5 is K or S

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, 21, or 22, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, 21, or 22, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%. 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%. 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%. 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al, shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al, shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%. 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to one, two, three, four, five, or six CDRs according to Kabat et al. shown in Table 1.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al, shown in Table 1. In some embodiments, the anti-TCRβV antibody molecule. e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al. (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a heavy chain variable region of an antibody described herein, e.g., an antibody chosen from any one of A-H.1 to A-H.85, e.g., A-H.1, A-H.2 or A-H.68, or as described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al, shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al, shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al (e.g., at least one, two, three, four, five, or six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al, shown in Table 1.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., by ImMunoGeneTics (IMGT) numbering system or as described in Table 1.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions, or by ImMunoGeneTics (IMGT) numbering system.

In some embodiments, a combined CDR as set out in Table 1 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 1.

In some embodiments, e.g., an embodiment comprising a variable region, a CDR (e.g., a combined CDR. Chothia CDR or Kabat CDR, or by ImMunoGene Tics (IMGT) numbering system), or other sequence referred to herein, e.g., in Table 1, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1. HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO. 8; and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W, position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβ V12 Antibodies

In one aspect, provided herein is an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In some embodiments, the heavy chain constant region comprises an amino sequence set forth in Table 3, 21, or 22, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In some embodiments, the light chain constant region comprises an amino sequence set forth in Table 3, 21, or 22, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, molecule includes all six CDRs from an antibody described herein, e.g., an antibody as described in Table 2, or encoded by the nucleotide sequence in Table 2, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al, shown in Table 2.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al, shown in Table 2.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%. 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to one, two, three, four, five, or six CDRs according to Kabat et al. shown in Table 2.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al, shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 2, e.g., the same canonical structures as at least loop 1 and/or loop 2 of the heavy and/or light chain variable domains of an antibody described herein. See, e.g., Chothia et al. (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al, shown in Table 2.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al, shown in Table 2.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al, shown in Table 2.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to all six CDRs according to Chothia et al, shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 2.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 2.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 2.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 2) from the heavy and light chain variable regions of an antibody described herein, e.g., an antibody as described in Table 2, or encoded by the nucleotide sequence in Table 2; or encoded by the nucleotide sequence in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 2. In some embodiments, the anti-TCRβV antibody molecule, e.g., anti-TCRβ V12 antibody molecule may include any CDR described herein.

In some embodiments, a combined CDR as set out in Table 1 is a CDR that comprises a Kabat CDR and a Chothia CDR or by ImMunoGeneTics (IMGT) numbering system.

In some embodiments, the anti-TCRβV antibody molecule, e e.g., anti-TCRβ V12 antibody molecule, molecule includes a combination of CDRs or hypervariable loops identified as combined CDRs in Table 1. In some embodiments, the anti-TCRβV antibody molecule, e.g., anti-TCRβ V12 antibody molecule, can contain any combination of CDRs or hypervariable loops according the “combined” CDRs are described in Table 1.

In some embodiments, the anti-TCRβV antibody molecule, e.g., anti-TCRβ V12 antibody molecule includes a combination of CDRs or hypervariable loops defined according to the Kabat et al, and Chothia et al., by ImMunoGeneTics (IMGT) numbering system, or as described in Table 1.

In some embodiments, the anti-TCRβV antibody molecule, e.g., anti-TCRβ V12 antibody molecule can contain any combination of CDRs or hypervariable loops according to the Kabat and Chothia definitions, or by ImMunoGeneTics (IMGT) numbering system.

In some embodiments, e.g., an embodiment comprising a variable region, a CDR (e.g., a combined CDR, Chothia CDR or Kabat CDR, or by ImMunoGeneTies (IMGT) numbering system), or other sequence referred to herein, e.g., in Table 2, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V12 antibody molecule includes. (i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24 or SEQ ID NO: 25.

In some embodiments, the anti-TCRβV antibody molecule, e.g., anti-TCRβ V12 antibody molecule comprises: (i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or (ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises: (1) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or (ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises: (i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a HC CDR1 amino acid sequence of SEQ ID NO. 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or (ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO. 58, or a HC CDR3 amino acid sequence of SEQ ID NO: 59.

In some embodiments, the light or the heavy chain variable framework (e.g., the region encompassing at least FR1, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence: (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

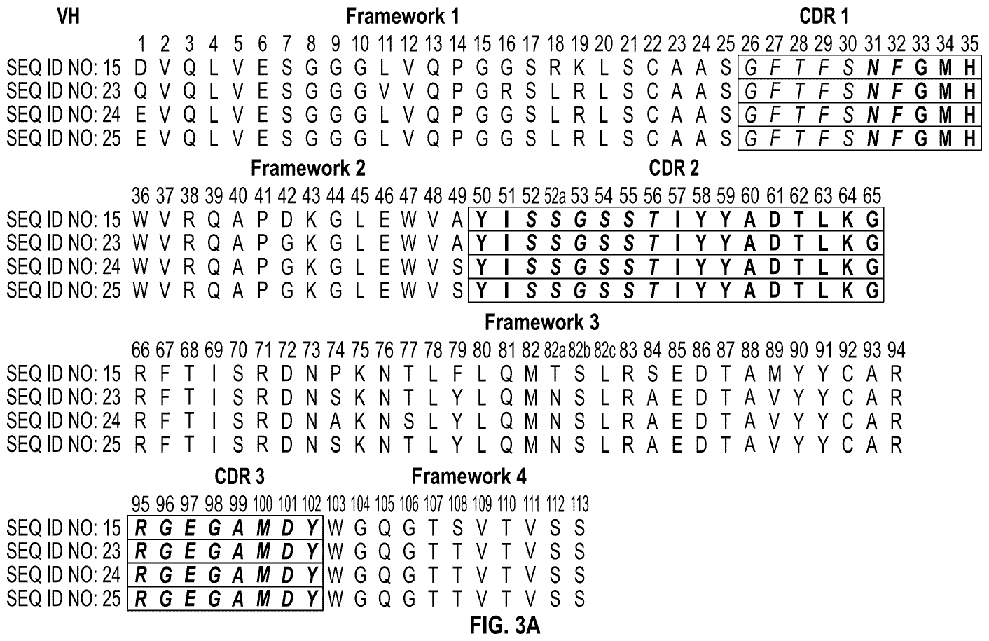

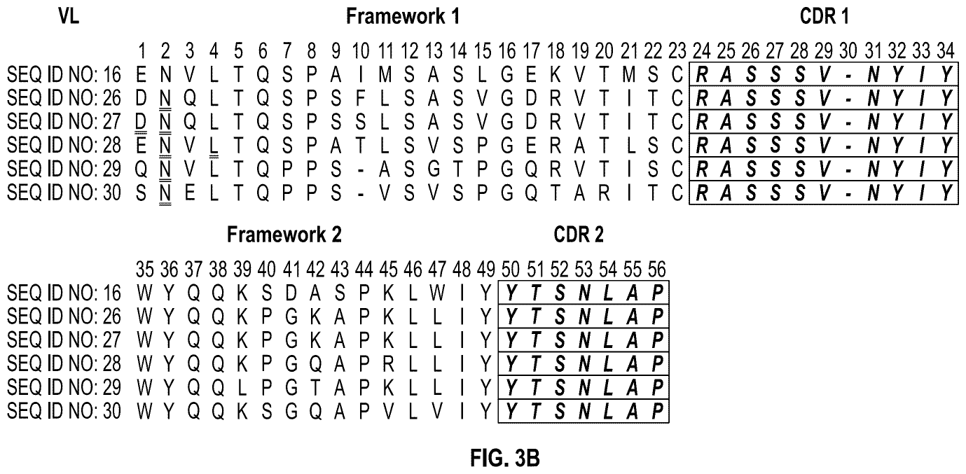

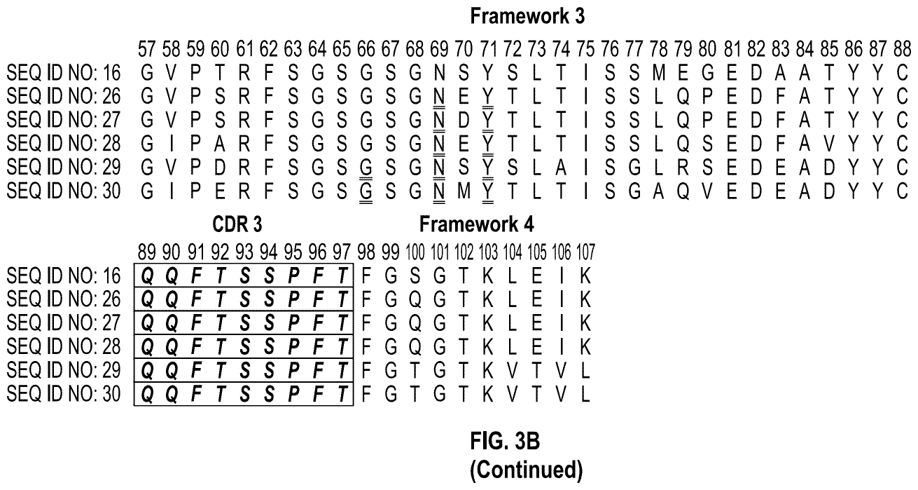

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 2. e.g., the amino acid sequence of the FR region in the entire variable region, e.g., shown in FIGS. 3A and 3B, or in SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein e.g., the amino acid sequence of the FR region in the entire variable region, e.g., shown in FIGS. 3A and 3B, or in SEQ ID NOs: 26-30.

In some embodiments, the anti-TCRβV antibody molecule, e.g., anti-TCRβ V12 antibody molecule includes one, two, three, or four heavy chain framework regions shown in FIG. 3A, or a sequence substantially identical thereto.

In some embodiments, the anti-TCRβV antibody molecule, e.g., anti-TCRβ V12 antibody molecule includes one, two, three, or four light chain framework regions shown in FIG. 3B, or a sequence substantially identical thereto. In some embodiments, the anti-TCRβV antibody molecule, e.g., anti-TCRβ V12 antibody molecule comprises the light chain framework region 1 e.g., as shown in FIG. 3B. In some embodiments, the anti-TCRβV antibody molecule, e.g., anti-TCRβ V12 antibody molecule comprises the light chain framework region 2 e.g., as shown in FIG. 3B. In some embodiments, the anti-TCRβV antibody molecule, e.g., anti-TCRβV12 antibody molecule comprises the light chain framework region 3, e.g., as shown in FIG. 3B. In some embodiments, the anti-TCRβV antibody molecule, e.g., anti-TCRβ V12 antibody molecule comprises the light chain framework region 4, e.g., as shown in FIG. 3B.

In some embodiments, the anti-TCRβV antibody molecule, e.g., anti-TCRβ V12 antibody molecule comprises a light chain comprising a framework region, e.g., framework region 1 (FR1), comprising a change, e.g., a substitution (e.g., a conservative substitution) at one or more, e.g., all, position as described herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution. Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering. e.g., an Alanine to Aspartic Acid substitution, and a substitution at position 2 according to Kabat numbering. e.g., an Isoleucine to Asparagine substitution. Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V12 antibody molecule comprises a light chain comprising a framework region, e.g., framework region 3 (FR3), comprising a change, e.g., a substitution (e.g., a conservative substitution) at one or more, e.g., all, position as described herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising. (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution, and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1, e.g., as shown in FIG. 3A. In some embodiments, the anti-TCRβV antibody molecule, e.g., anti-TCRβ V12 antibody molecule comprises the heavy chain framework region 2, e.g., as shown in FIG. 3A. In some embodiments, the anti-TCRβV antibody molecule, e.g., anti-TCRβ V12 antibody molecule comprises the heavy chain framework region 3, e.g., as shown in FIG. 3A. In some embodiments, the anti-TCRβV antibody molecule, e.g., anti-TCRβ V12 antibody molecule comprises the heavy chain framework region 4, e.g., as shown in FIG. 3A. In some embodiments, the anti-TCRβV antibody molecule, e.g., anti-TCRβ V12 antibody molecule comprises the heavy chain framework regions 1-4, e.g., SEQ ID NOS: 20-23, or as shown in FIG. 3A. In some embodiments, the anti-TCRβV antibody molecule, e.g., anti-TCRβ V12 antibody molecule comprises the light chain framework regions 1-4, e.g., SEQ ID NOs: 26-30, or as shown in FIG. 3B.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30, or as shown in FIGS. 3A and 3B.

In some embodiments, the heavy or light chain variable domain, or both, of, the anti-TCRβV antibody molecule, e.g., anti-TCRβ V12 antibody molecule includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody as described in Table 2, or encoded by the nucleotide sequence in Table 2; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 2, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 2. In another embodiment, the anti-TCRβV antibody molecule, e.g., anti-TCRβ V12 antibody molecule includes a VH and/or VL domain encoded by a nucleic acid having a nucleotide sequence as set forth in Table 2, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2.

In some embodiments, the anti-TCRβV antibody molecule, e.g., anti-TCRβ V12 antibody molecule comprises: a VH domain comprising an amino acid sequence chosen from the amino acid sequence of SEQ ID NO: 23, SEQ ID NO:24 or SEQ ID NO:25, an amino acid sequence at least about 85%, 90%, 95%, 99% or more identical to the amino acid sequence SEQ ID NO: 23, SEQ ID NO:24 or SEQ ID NO:25, or an amino acid sequence which differs by no more than 1, 2, 5, 10, or 15 amino acid residues from the amino acid sequence of SEQ ID NO: 23, SEQ ID NO:24 or SEQ ID NO:25; and/or a VL domain comprising an amino acid sequence chosen from the amino acid sequence of SEQ ID NO: 26, SEQ ID NO: 27, SEQ ID NO: 28, SEQ ID NO: 29 or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29 or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29 or SEQ ID NO: 30. In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO. 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO. 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO. 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO. 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab, F(ab′)₂. Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab, F(ab′) 2. Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a multispecific molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1. IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 296 (M to Y). 298 (S to T), 300 (T to E), 477 (H to K) and 478 (N to F) to alter Fc receptor binding (e.g., the mutated positions correspond to positions 132 (M to Y), 134 (S to T). 136 (T to E). 313 (H to K) and 314 (N to F) of SEQ ID NOs: 212 or 214; or positions 135 (M to Y), 137 (S to T). 139 (T to E). 316 (H to K) and 317 (N to F) of SEQ ID NOs: 215, 216, 217 or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies are provided in Table 2. In some embodiments, the anti-TCRβ V12 is antibody B, e.g., humanized antibody B (antibody B-H), as provided in Table 2. In some embodiments, the anti-TCRβV antibody comprises one or more (e.g., all three) of a LC CDR1, LC CDR2, and LC CDR3 provided in Table 2, and/or one or more (e.g., all three) of a HC CDR1, HC CDR2, and HC CDR3 provided in Table 2, or a sequence with at least 95% sequence identity thereto. In some embodiments, antibody B comprises a variable heavy chain (VH) and/or a variable light chain (VL) provided in Table 2, or a sequence with at least 95% sequence identity thereto.

In some embodiments, the anti-TCRVB 12 antibody molecule (e.g., anti-TCRVB 12-3 or anti-TCRVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VL of B-H.1A, B-H.1B, B-H.1C, B-H.1D, B-H.1E, B-H.1F, B-H.1G. B-H.1H. B-H.1. B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VH of B-H.1A. B-H.1B. B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto; and a VL of B-H.1A, B-H.1B, B-H.1C, B-H.1D, B-H.1E. B-H.1F, B-H.1G, B-H.1H. B-H.1. B-H.2, B-H.3. B-H.4, B-H.5, or B-H.6, or a sequence with at least 80%, 85%, 90%, 95%, 96%, 97%, 98%, 99% or more identity thereto.

### Anti-TCRβ V5 Antibodies

In one aspect, provided herein is an anti-TCRβV antibody molecule that binds to human TCRβ V5. In some embodiments, the TCRβ V5 subfamily comprises TCRβ V5-5*01, TCRβ V5-6*01, TCRβ V5-4*01. TCRβ V5-8*01, TCRβ V5-1*01, or a variant thereof.

Exemplary anti-TCRβ V5 antibodies are provided in Table 10B. In some embodiments, the anti-TCRβ V5 is antibody C, e.g., humanized antibody C (antibody C-H), as provided in Table 10B. In some embodiments, the anti-TCRβV antibody comprises one or more (e.g., all three) of a LC CDR1, LC CDR2, and LC CDR3 provided in Table 10B; and/or one or more (e.g., all three) of a HC CDR1, HC CDR2, and HC CDR3 provided in Table 10B, or a sequence with at least 95% sequence identity thereto. In some embodiments, antibody C comprises a variable heavy chain (VH) and/or a variable light chain (VL) provided in Table 10B, or a sequence with at least 95% sequence identity thereto.

Exemplary anti-TCRβ V5 antibodies are provided in Table 11. In some embodiments, the anti-TCRB V5 is antibody E, e.g., humanized antibody E (antibody E-H), as provided in Table 11. In some embodiments, the anti-TCRβV antibody comprises one or more (e.g., all three) of a LC CDR1, LC CDR2, and LC CDR3 provided in Table 11; and/or one or more (e.g., all three) of a HC CDR1, HC CDR2, and HC CDR3 provided in Table 11, or a sequence with at least 95% sequence identity thereto. In some embodiments, antibody E comprises a variable heavy chain (VH) and/or a variable light chain (VL) provided in Table 11, or a sequence with at least 95% sequence identity thereto.

In some embodiments, antibody E comprises a heavy chain comprising the amino acid sequence of SEQ ID NO: 3284 and/or a light chain comprising the amino acid sequence of SEQ ID NO: 3285, or a sequence with at least 95% sequence identity thereto.

In some embodiments, the anti-TCRβ V5 antibody molecule comprises a VH and/or a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11, or a sequence with at least 80%, 85%, 90%, 95%, 96%, 97%, 98%, 99% or more identity thereto

In some embodiments, the anti-TCRβ V5 antibody molecule comprises a VH and a VL of an antibody described in Table 11, or a sequence with at least 80%, 85%, 90%, 95%, 96%, 97%, 98%, 99% or more identity thereto.

### Anti-TCRβ V10 Antibodies

In one aspect, provided herein is an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβ V10 antibodies are provided in Table 12. In some embodiments, the anti-TCRβ V10 is antibody D, e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% sequence identity thereto. In some embodiments, antibody D comprises a variable heavy chain (VH) and/or a variable light chain (VL) provided in Table 12, or a sequence with at least 95% sequence identity thereto.

In some embodiments, the anti-TCRβ V10 antibody molecule comprises a VH or a VL of an antibody described in Table 12, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

Additional anti-TCRVβ Antibodies

Additional exemplary anti-TCRβV antibodies are provided in Table 13. In some embodiments, the anti-TCRβV antibody is a humanized antibody, e.g., as provided in Table 13. In some embodiments, the anti-TCRβV antibody comprises one or more (e.g., all three) of a LC CDR1, LC CDR2, and LC CDR3 provided in Table 13; and/or one or more (e.g., all three) of a HC CDR1, HC CDR2, and HC CDR3 provided in Table 13, or a sequence with at least 95% sequence identity thereto. In some embodiments, the anti-TCRβV antibody comprises a variable heavy chain (VH) and/or a variable light chain (VL) provided in Table 13, or a sequence with at least 95% sequence identity thereto.

### Antibody-Like Frameworks or Scaffolds

A wide variety of antibody/immunoglobulin frameworks or scaffolds can be employed in the anti-TCRvb antibody molecules as described herein or multifunctional formats thereof so long as the resulting polypeptide includes at least one binding region which specifically binds to the target antigen, e.g., a TCRvb, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRvb antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis. Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRvb antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRVβ Antibody Effector Function and Fc Variants

In some embodiments, an anti-TCRVβ antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including; antibody dependent cell-mediated cytotoxicity (ADCC), Antibody-dependent cellular phagocytosis (ADCP) and complement dependent cytotoxicity (CDC).

In some embodiments, an anti-TCRVβ antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ antibody comprising a variant Fc region has one or more of the following properties: (1) reduced effector function (e.g., reduced ADCC. ADCP and/or CDC): (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ antibody comprising a variant Fc region has reduced affinity to a human Fc receptor, e.g., FcγR I, FcγR II and/or FcγR III. In some embodiments, the anti-TCRVβ antibody comprising a variant Fc region comprises a human IgG1 region or a human IgG4 region.

In some embodiments, an anti-TCRVβ antibody comprising a variant Fc region activates and/or expands T cells, e.g., as described herein. In some embodiments, an anti-TCRVβ antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule.

Exemplary Fc region variants are provided in Table 14 and also disclosed in Saunders O, (2019) Frontiers in Immunology; vol 10, article 1296, the entire contents of which is hereby incorporated by reference.

In some embodiments, an anti-TCRVβ antibody as described herein comprises any one or all, or any combination of Fc region variants disclosed in Table 14.

In some embodiments, an anti-TCRVβ antibody as described herein comprises any one or all, or any combination of Fc region variants, e.g., mutations, disclosed in Table 14. In some embodiments, an anti-TCRVβ antibody as described herein comprise an Asn297Ala (N297A) mutation. In some embodiments, an anti-TCRVβ antibody as described herein comprise a Leu234Ala/Leu235Ala (LALA) mutation.

### Multifunctional Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule, e.g., a bispecific antibody molecule. In some embodiments, the multispecific molecule includes an anti-TCRVb antibody molecule as described herein.

Described herein, in certain embodiments, is a multifunctional polypeptide molecule comprising a first polypeptide chain, a second polypeptide chain, a third polypeptide chain, a fourth polypeptide chain, and at least one cytokine polypeptide or a functional fragment or a functional variant thereof, wherein the first polypeptide chain, the second polypeptide chain, the third polypeptide chain, and the fourth polypeptide chain are non-contiguous, wherein: (i) the first polypeptide chain comprising a first portion of a first T cell receptor variable beta (TCRβV)-binding moiety and a first dimerization module linked to the first portion of the first TCRβV-binding moiety; (ii) the second polypeptide chain comprising a second portion of the first TCRβV-binding moiety; (iii) the third polypeptide chain comprising a first portion of a second TCRβV-binding moiety and a second dimerization module linked to the first portion of the second TCRβV-binding moiety; and (iv) the fourth polypeptide chain comprising a second portion of the second TCRβV-binding moiety; and wherein the at least one cytokine polypeptide or a functional fragment or a functional variant thereof is covalently linked to the first polypeptide chain, the second polypeptide chain, the third polypeptide chain, the fourth polypeptide chain, or a combination thereof.

Described herein, in certain embodiments, is a multifunctional polypeptide molecule comprising a first polypeptide chain, a second polypeptide chain, a third polypeptide chain, and at least one cytokine polypeptide or a functional fragment or a functional variant thereof, wherein the first polypeptide chain, the second polypeptide chain, and the third polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; and (iii) the third polypeptide chain comprising a second dimerization module; and wherein the at least one cytokine polypeptide or a functional fragment or a functional variant thereof is covalently linked to the first polypeptide chain, the second polypeptide chain, the third polypeptide chain, or a combination thereof.

Described herein, in certain embodiments, is a multifunctional polypeptide molecule comprising a first polypeptide chain, a second polypeptide chain, a third polypeptide chain, and at least one cytokine polypeptide or a functional fragment or a functional variant thereof, wherein the first polypeptide chain, the second polypeptide chain, and the third polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; and (iii) the third polypeptide chain comprising a second dimerization module: wherein the at least one cytokine polypeptide or a functional fragment or a functional variant thereof is covalently linked to the first polypeptide chain, the second polypeptide chain, the third polypeptide chain, or a combination thereof; and wherein the multifunctional polypeptide molecule does not comprise an additional TCRβV-binding moiety except the first TCRβV-binding moiety.

In some embodiments, the first portion of the first TCRβV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRβV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRβV-binding moiety. In some embodiments, the first dimerization module is linked to the C-terminus of the first portion of the first TCRβV-binding moiety. In some embodiments, wherein the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments. (a) the N-terminus of the first polypeptide chain is linked to a first cytokine polypeptide or a functional fragment or a functional variant thereof; the C-terminus of the first polypeptide chain is linked to a second cytokine polypeptide or a functional fragment or a functional variant thereof; or a combination thereof; (b) the N-terminus of the second polypeptide chain is linked to a third cytokine polypeptide or a functional fragment or a functional variant thereof; the C-terminus of the second polypeptide chain is linked to a fourth cytokine polypeptide or a functional fragment or a functional variant thereof; or a combination thereof; (c) the N-terminus of the third polypeptide chain is linked to a fifth cytokine polypeptide or a functional fragment or a functional variant thereof; the C-terminus of the third polypeptide chain is linked to a sixth cytokine polypeptide or a functional fragment or a functional variant thereof; or a combination thereof; (d) the N-terminus of the fourth polypeptide chain is linked to a seventh cytokine polypeptide or a functional fragment or a functional variant thereof; the C-terminus of the fourth polypeptide chain is linked to an eighth cytokine polypeptide or a functional fragment or a functional variant thereof; or a combination thereof; or (e) a combination thereof.

In some embodiments, (a-1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; and (a-2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; (b-1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; and (b-2) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; (c-1) the N-terminus of the first polypeptide chain is linked to the first cytokine polypeptide or a functional fragment or a functional variant thereof, the C-terminus of the first polypeptide chain is linked to the second cytokine poly peptide or a functional fragment or a functional variant thereof; or a combination thereof; and (c-2) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof, or a combination thereof; (d-1) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; and (d-2) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; (e-1) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; and (e-2) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof; or a combination thereof; or (f-1) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; and (f-2) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof; or a combination thereof.

In some embodiments, (a-1) the N-terminus of the first polypeptide chain is linked to the first cytokine polypeptide or a functional fragment or a functional variant thereof; the C-terminus of the first poly peptide chain is linked to the second cytokine poly peptide or a functional fragment or a functional variant thereof; or a combination thereof; (a-2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; and (a-3) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; (b-1) the N-terminus of the first polypeptide chain is linked to the first cytokine polypeptide or a functional fragment or a functional variant thereof, the C-terminus of the first polypeptide chain is linked to the second cytokine poly peptide or a functional fragment or a functional variant thereof; or a combination thereof; (b-2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof, or a combination thereof; and (b-3) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof; or a combination thereof; or (c-1) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; (c-2) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; and (c-3) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof; or a combination thereof.

In some embodiments. (1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; (2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; (3) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; and (4) the N-terminus of the fourth polypeptide chain is linked to the seventh cytokine polypeptide or a functional fragment or a functional variant thereof; the C-terminus of the fourth polypeptide chain is linked to the eighth cytokine polypeptide or a functional fragment or a functional variant thereof; or a combination thereof.

In some embodiments, the first cytokine polypeptide, the second cytokine polypeptide, or a combination thereof is within a single contiguous polypeptide chain of the first polypeptide chain, the third cytokine polypeptide, the fourth cytokine polypeptide, or a combination thereof is within a single contiguous polypeptide chain of the second polypeptide chain, the fifth cytokine polypeptide, the sixth cytokine polypeptide, or a combination thereof is within a single contiguous polypeptide chain of the third polypeptide chain, the seventh cytokine polypeptide, the eighth cytokine polypeptide, or a combination thereof is within a single contiguous polypeptide chain of the fourth polypeptide chain, or a combination thereof.

In some embodiments, (a) the N-terminus of the first polypeptide chain is linked to a first cytokine polypeptide or a functional fragment or a functional variant thereof; the C-terminus of the first polypeptide chain is linked to a second cytokine polypeptide or a functional fragment or a functional variant thereof; or a combination thereof; (b) the N-terminus of the second polypeptide chain is linked to a third cytokine polypeptide or a functional fragment or a functional variant thereof; the C-terminus of the second polypeptide chain is linked to a fourth cytokine polypeptide or a functional fragment or a functional variant thereof; or a combination thereof; (c) the N-terminus of the third polypeptide chain is linked to a fifth cytokine polypeptide or a functional fragment or a functional variant thereof; the C-terminus of the third polypeptide chain is linked to a sixth cytokine polypeptide or a functional fragment or a functional variant thereof; or a combination thereof; or (d) a combination thereof.

In some embodiments. (a-1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; and (a-2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; (b-1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; and (b-2) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof; or (c-1) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof, or a combination thereof; and (c-2) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof.

In some embodiments. (1) the N-terminus of the first polypeptide chain is linked to the first cytokine polypeptide or a functional fragment or a functional variant thereof; the C-terminus of the first polypeptide chain is linked to the second cytokine polypeptide or a functional fragment or a functional variant thereof; or a combination thereof; (2) the N-terminus of the second polypeptide chain is linked to the third cytokine polypeptide or a functional fragment or a functional variant thereof; the C-terminus of the second polypeptide chain is linked to the fourth cytokine polypeptide or a functional fragment or a functional variant thereof; or a combination thereof; and (3) the N-terminus of the third polypeptide chain is linked to the fifth cytokine polypeptide or a functional fragment or a functional variant thereof; the C-terminus of the third polypeptide chain is linked to the sixth cytokine polypeptide or a functional fragment or a functional variant thereof; or a combination thereof.

In some embodiments, the first cytokine polypeptide, the second cytokine polypeptide, or a combination thereof is within a single contiguous polypeptide chain of the first polypeptide chain, the third cytokine polypeptide, the fourth cytokine polypeptide, or a combination thereof is within a single contiguous polypeptide chain of the second polypeptide chain, the fifth cytokine polypeptide, the sixth cytokine polypeptide, or a combination thereof is within a single contiguous polypeptide chain of the third polypeptide chain, or a combination thereof.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide chain, a linker between the at least one cytokine polypeptide or a functional fragment or a functional variant thereof and the second polypeptide chain, a linker between the at least one cytokine polypeptide or a functional fragment or a functional variant thereof and the third polypeptide chain, a linker between the at least one cytokine polypeptide or a functional fragment or a functional variant thereof and the fourth polypeptide chain, or a combination thereof.

In some embodiments, the multifunctional polypeptide molecule as described herein further comprises comprising a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the first polypeptide chain, a linker between the at least one cytokine polypeptide or a functional fragment or a functional variant thereof and the second polypeptide chain, a linker between the at least one cytokine polypeptide or a functional fragment or a functional variant thereof and the third polypeptide chain, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide chain, a second polypeptide chain, a third polypeptide chain, a fourth polypeptide chain, a first cytokine polypeptide or a functional fragment or a functional variant thereof, and a second cytokine polypeptide or a functional fragment or a functional variant thereof, wherein the first polypeptide chain, the second polypeptide chain, the third polypeptide chain, and the fourth polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; (iii) the third polypeptide chain comprising a first portion of a second TCRβV-binding moiety and a second dimerization module linked to the first portion of the second TCRβV-binding moiety; and (iv) the fourth polypeptide chain comprising a second portion of the second TCRβV-binding moiety; and wherein the first cytokine polypeptide or a functional fragment or a functional variant thereof is covalently linked to the C-terminus of the second polypeptide chain, and the second cytokine polypeptide or a functional fragment or a functional variant thereof is covalently linked to the C-terminus of the fourth polypeptide chain.

Described herein, in certain embodiments, is a multifunctional polypeptide molecule comprising a first polypeptide chain, a second polypeptide chain, a third polypeptide chain, a fourth polypeptide chain, a cytokine polypeptide or a functional fragment or a functional variant thereof, wherein the first polypeptide chain, the second polypeptide chain, the third poly peptide chain, and the fourth polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; (iii) the third polypeptide chain comprising a first portion of a second TCRβV-binding moiety and a second dimerization module linked to the first portion of the second TCRβV-binding moiety; and (iv) the fourth polypeptide chain comprising a second portion of the second TCRβV-binding moiety; and wherein the cytokine polypeptide or a functional fragment or a functional variant thereof is covalently linked to the C-terminus of the second polypeptide chain or the C-terminus of the fourth polypeptide chain.

Described herein, in certain embodiments, is a multifunctional polypeptide molecule comprising a first polypeptide chain, a second polypeptide chain, a third polypeptide chain, a fourth polypeptide chain, a cytokine polypeptide or a functional fragment or a functional variant thereof, wherein the first polypeptide chain, the second polypeptide chain, the third polypeptide chain, and the fourth polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; (iii) the third polypeptide chain comprising a first portion of a second TCRβV-binding moiety and a second dimerization module linked to the first portion of the second TCRβV-binding moiety; and (iv) the fourth polypeptide chain comprising a second portion of the second TCRβV-binding moiety; and wherein the cytokine polypeptide or a functional fragment or a functional variant thereof is covalently linked to the C-terminus of the first polypeptide chain or the C-terminus of the third polypeptide chain.

Described herein, in certain embodiments, is a multifunctional polypeptide molecule comprising a first polypeptide chain, a second polypeptide chain, a third polypeptide chain, and a cytokine polypeptide or a functional fragment or a functional variant thereof, wherein the first polypeptide chain, the second polypeptide chain, and the third polypeptide chain are non-contiguous, wherein: (i) the first polypeptide chain comprising a first portion of a first TCRβV-binding moiety and a first dimerization module linked to the first portion of the first TCRβV-binding moiety; (ii) the second polypeptide chain comprising a second portion of the first TCRβV-binding moiety; and (iii) the third polypeptide chain comprising a second dimerization module: wherein the at least one cytokine polypeptide or a functional fragment or a functional variant thereof is covalently linked to the N terminus of the third polypeptide chain; and wherein the multifunctional polypeptide molecule does not comprise an additional TCRβV-binding moiety except the first TCRβV-binding moiety.

In some embodiments, the first portion of the first TCRβV-binding moiety comprises a first VH and a first CH1 linked to the first VH. In some embodiments, the first CH1 is linked to the C-terminus of the first VH.

In some embodiments, the second portion of the first TCRβV-binding moiety comprises a first VL and a first CL linked to the first VL. In some embodiments, first CL is linked to the C-terminus of the first VL.

In some embodiments, the first dimerization module is linked to the first portion of the first TCRβV-binding moiety. In some embodiments, the first dimerization module is linked to the C-terminus of the first portion of the first TCRβV-binding moiety. In some embodiments, the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide chain, a linker between the at least one cytokine polypeptide or a functional fragment or a functional variant thereof and the second polypeptide chain, a linker between the at least one cytokine polypeptide or a functional fragment or a functional variant thereof and the third polypeptide chain, a linker between the at least one cytokine polypeptide or a functional fragment or a functional variant thereof and the fourth polypeptide chain, or a combination thereof. In some embodiments, the multifunctional polypeptide molecule as described herein further comprises a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the third polypeptide chain, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRβV-binding moiety, the second TCRβV-binding moiety, or a combination thereof comprises any one selected from the group consisting of a Fab, F(ab′) 2, Fv, a single chain Fv (scFv), a single domain antibody, a diabody (dAb), a camelid antibody and a combination thereof. In some embodiments, the first TCRβV-binding moiety, the second TCRβV-binding moiety, or a combination thereof comprises a scFv or a Fab.

In some embodiments, the multifunctional polypeptide molecule does not comprise an additional antigen-binding moiety except the TCRβV-binding moiety. In some embodiments, the multifunctional polypeptide molecule further comprise an additional antigen-binding moiety that is not the TCRβV-binding moiety.

Described herein, in certain embodiments, is a multifunctional polypeptide molecule comprising a first poly peptide chain, a second polypeptide chain, and at least one cytokine poly peptide or a functional fragment or a functional variant thereof, wherein the first polypeptide chain and the second polypeptide chain are non-contiguous, wherein: (i) the first polypeptide chain comprising a first TCRβV-binding moiety and a first dimerization module linked to the C-terminus of the first TCRβV-binding moiety, wherein the first TCRβV-binding moiety comprises a first VL and a first VH; and (ii) the second polypeptide chain comprising a second TCRβV-binding moiety and a second dimerization module linked to the C-terminus of the second TCRβV-binding moiety: wherein the at least one cytokine polypeptide or a functional fragment or a functional variant thereof is covalently linked to the first polypeptide chain, the second polypeptide chain, or a combination thereof; wherein the first TCRβV-binding moiety, the second TCRβV-binding moiety, or a combination thereof comprises a scFv; and wherein the multifunctional polypeptide molecule does not comprise an additional antigen-binding moiety except the first TCRβV-binding moiety and the second TCRβV-binding moiety.

Described herein, in certain embodiments, is a multifunctional polypeptide molecule comprising a first polypeptide chain, a second polypeptide chain, and at least one cytokine polypeptide or a functional fragment or a functional variant thereof, wherein the first polypeptide chain and the second polypeptide chain are non-contiguous, wherein: (i) the first polypeptide chain comprising a first TCRβV-binding moiety and a first dimerization module linked to the C-terminus of the first TCRβV-binding moiety, wherein the first TCRβV-binding moiety comprises a first VL and a first VH; and (ii) the second polypeptide chain comprising a second dimerization module: wherein the at least one cytokine polypeptide or a functional fragment or a functional variant thereof is covalently linked to the first polypeptide chain, the second polypeptide chain, or a combination thereof; wherein the first TCRβV-binding moiety comprises a scFv: wherein the multifunctional poly peptide molecule does not comprise an additional antigen-binding moiety except the first TCRβV-binding moiety; and wherein the multifunctional polypeptide molecule does not comprise an additional TCRβV-binding moiety except the first TCRβV-binding moiety.

In some embodiments, (a) the N-terminus of the first polypeptide chain is linked to a first cytokine polypeptide or a functional fragment or a functional variant thereof; the C-terminus of the first polypeptide chain is linked to a second cytokine polypeptide or a functional fragment or a functional variant thereof; or a combination thereof; (b) the N-terminus of the second polypeptide chain is linked to a third cytokine polypeptide or a functional fragment or a functional variant thereof; the C-terminus of the second polypeptide chain is linked to a fourth cytokine polypeptide or a functional fragment or a functional variant thereof; or a combination thereof; or (e) a combination thereof.

In some embodiments, the first cytokine polypeptide, the second cytokine polypeptide, or a combination thereof is within a single contiguous polypeptide chain of the first polypeptide chain, the third cytokine polypeptide, the fourth cytokine polypeptide, or a combination thereof is within a single contiguous polypeptide chain of the second polypeptide chain, or a combination thereof.

In some embodiments, the multifunctional polypeptide molecule as described herein further comprises a linker between the first TCRβV-binding moiety and the first dimerization module, a linker between the second TCRβV-binding moiety and the second dimerization module, a linker between the at least one cytokine polypeptide or a functional fragment or a functional variant thereof and the first polypeptide chain, a linker between the at least one cytokine polypeptide or a functional fragment or a functional variant thereof and the second polypeptide chain, or a combination thereof.

In some embodiments, the multifunctional polypeptide molecule as described herein further comprises a linker between the first TCRβV-binding moiety and the first dimerization module, a linker between the at least one cytokine polypeptide or a functional fragment or a functional variant thereof and the first polypeptide chain, a linker between the at least one cytokine polypeptide or a functional fragment or a functional variant thereof and the second polypeptide chain, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptide. In some embodiments, the multifunctional polypeptide molecule comprises at least three of the cytokine polypeptide. In some embodiments, the multifunctional polypeptide molecule comprises at least four of the cytokine polypeptide. In some embodiments, the multifunctional polypeptide molecule comprises at least five of the cytokine polypeptide. In some embodiments, the multifunctional polypeptide molecule comprises at least six of the cytokine polypeptide. In some embodiments, the multifunctional polypeptide molecule comprises at least seven of the cytokine polypeptide. In some embodiments, the multifunctional polypeptide molecule comprises at least eight of the cytokine polypeptide. In some embodiments, the multifunctional polypeptide molecule comprises two of the cytokine polypeptide. In some embodiments, the multifunctional polypeptide molecule comprises three of the cytokine polypeptide. In some embodiments, the multifunctional polypeptide molecule comprises four of the cytokine polypeptide. In some embodiments, the multifunctional polypeptide molecule comprises five of the cytokine polypeptide. In some embodiments, the multifunctional polypeptide molecule comprises six of the cytokine polypeptide. In some embodiments, the multifunctional polypeptide molecule comprises seven of the cytokine polypeptide. In some embodiments, the multifunctional polypeptide molecule comprises eight of the cytokine polypeptide. In some embodiments, the multifunctional polypeptide molecule comprises two of the cytokine polypeptide, each of which is linked to the first polypeptide chain and the second polypeptide chain: the first polypeptide chain and the third polypeptide chain; the first polypeptide chain and the fourth polypeptide chain; the second and the third polypeptide chain: the second polypeptide chain and the fourth polypeptide chain; or the third polypeptide chain and the fourth polypeptide chain, respectively. In some embodiments, the multifunctional polypeptide molecule comprises three of the cytokine polypeptide, each of which is linked to the first polypeptide chain, the second polypeptide chain, and the third polypeptide chain; the first polypeptide chain, the second polypeptide chain, and the fourth poly peptide chain: the first poly peptide chain, the third polypeptide chain, and the fourth polypeptide chain; or the second polypeptide chain, the third polypeptide chain, and the fourth polypeptide chain, respectively. In some embodiments, the multifunctional polypeptide molecule comprises four of the cytokine polypeptide, each of which is linked to the first polypeptide chain, the second polypeptide chain, the third polypeptide chain, and the fourth polypeptide chain, respectively. In some embodiments, the cytokine polypeptide is not linked to the polypeptides that comprise the first TCRβV-binding moiety.

In some embodiments, the at least one cytokine polypeptide is selected from the group consisting of interleukin-2 (IL-2) or a fragment or a functional fragment or a functional variant thereof, interleukin-7 (IL-7) or a fragment or a functional fragment or a functional variant thereof, interleukin-12 (IL-12) or a fragment or a functional fragment or a functional variant thereof, interleukin-15 (IL-15) or a fragment or a functional fragment or a functional variant thereof, interleukin-18 (IL-18) or a fragment or a functional fragment or a functional variant thereof, interleukin-21 (IL-21) or a fragment or a functional fragment or a functional variant thereof, or interferon gamma or a fragment or a functional fragment or a functional variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of, a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold. 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold. 3 fold, 4 fold, S fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO. 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42. SEQ ID NO: 3645, SEQ ID NO: 3646, SEQ ID NO: 3647, SEQ ID NO:3648, or SEQ ID NO: 3649.

In some embodiments, the first TCRβV-binding moiety, the second TCRβV-binding moiety, or a combination thereof binds to one or more of a TCRβV subfamily selected from the group consisting of: (i) TCRβ V2 subfamily comprising TCRβ V2*01; (ii) TCRβ V3 subfamily comprising TCRβ V3-1*01; (iii) TCRβ V4 subfamily comprising one or more selected from TCRβ V4-1, TCRβ V4-2, and TCRβ V4-3; (iv) TCRβ V5 subfamily comprising one or more selected from TCRβ V5-6*01, TCRβ V5-4*01, TCRβ V5-1*01, and TCRβ V5-8*01; (v) the TCRβ V6 subfamily comprising one or more selected from TCRβ V6-4*01. TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01; (vi) TCRβ V9 subfamily; (vii) TCRβ V10 subfamily comprising one or more selected from TCRβ V10-1*01, TCRβ V10-J*02, TCRβ V10-3*01, and TCRβ V10-2*01; (viii) TCRβ V11 subfamily comprising TCRβ V11-2; (ix) TCRβ V12 subfamily comprising one or more selected from TCRβ V12-4*01, TCRβ V12-3*01, and TCRβ V12-5*01; (x) TCRβ V13 subfamily comprising TCRβ V13*01; (xi) TCRβ V16 subfamily comprising TCRβ V16*01; (xii) TCRβ V19 subfamily comprising one or more selected from TCRβ V19*01 and TCRβ V19*02; (xiii) TCRβ V21 subfamily. (xiv) TCRβ V23 subfamily. (xv) TCRβ V27 subfamily; and (xvi) TCRβ V28 subfamily.

In some embodiments, the first TCRβV-binding moiety and the second TCRβV-binding moiety are same. In some embodiments, the first TCRβV-binding moiety and the second TCRβV-binding moiety are different.

In some embodiments, the first TCRβV-binding moiety and the second TCRβV-binding moiety binds: (i) one or more of a TCRβ V6 subfamily member and one or more of a TCRβ V10 subfamily member, respectively; (ii) one or more of a TCRβ V6 subfamily member and one or more of a TCRβ V5 subfamily member, respectively; (iii) one or more of a TCRβ V6 subfamily member and one or more of a TCRβ V12 subfamily member, respectively; (iv) one or more of a TCRβ V10 subfamily member and one or more of a TCRβ V5 subfamily member, respectively; (v) one or more of a TCRβ V10 subfamily member and one or more of a TCRβ V12 subfamily member, respectively; or (vi) one or more of a TCRβ V5 subfamily member and one or more of a TCRβ V12 subfamily member, respectively.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1; (ii) a LC CDR1, a LC CDR2, and a LC CDR3 having any one of the CDR1, CDR2, and CDR3 the sequences listed in Table 1; or (ii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1, respectively; (ii) a LC CDR1, a LC CDR2, and a LC CDR3 having any one of the CDR1, CDR2, and CDR3 the sequences listed in Table 1, respectively; or (iii) a combination thereof.

In some embodiments, the first TCRβV-binding moiety, the second TCRβV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRβV-binding moiety, the second TCRβV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the VH comprises the FR3 comprising (i) a Threonine at position 73 according to Kabat numbering; (ii) a Glycine a position 94 according to Kabat numbering; or (iii) a combination thereof. In some embodiments, the VL comprises the FR1 comprising a Phenylalanine at position 10 according to Kabat numbering. In some embodiments, the VL comprises the FR2 comprising (i) a Histidine at position 36 according to Kabat numbering; (ii) an Alanine at position 46 according to Kabat numbering; or (iii) a combination thereof. In some embodiments, the VL comprises the FR3 comprising a Phenylalanine at position 87 according to Kabat numbering.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2; (ii) a LC CDR1, a LC CDR2, and a LC CDR3 having any one of the CDR1, CDR2, and CDR3 sequences listed in Table 2; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively. (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2, respectively; (ii) a LC CDR1, a LC CDR2, and a LC CDR3 having any one of the CDR1, CDR2, and CDR3 sequences listed in Table 2, respectively; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2; (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 that have the sequence of a FR1, a FR2, a FR3, and a FR4 of a humanized B-H LC of Table 2; (ii) a VL comprising a FR comprising a FR1, a FR2, a FR3, and a FR4 that have the sequence of a FR1, a FR2, a FR3, and a FR4 of a humanized B-H LC of Table 2; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively. (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 that have the sequence of a FR1, a FR2, a FR3, and a FR4 of a humanized B-H LC of Table 2, respectively; (ii) a VL comprising a FR comprising a FR1, a FR2, a FR3, and a FR4 that have the sequence of a FR1, a FR2, a FR3, and a FR4 of a humanized B-H LC of Table 2, respectively; or (iii) a combination thereof.

In some embodiments, the first TCRβV-binding moiety, the second TCRβV-binding moiety, or a combination thereof comprises: (i) a VH comprising a sequence having at least 75%, 80%, 85%, 90%, 95%, 99%, 99.5%, 99.9%, or 100% sequence identity to the VH sequence of a humanized Antibody B-H listed in Table 2; (ii) a VL comprising a sequence having at least at least 75%, 80%, 85%, 90%, 95%, 99%, 99.5%, 99.9%, or 100% sequence identity to the VL sequence of a humanized Antibody B-H listed in Table 2; or (iii) a combination thereof. In some embodiments, the first TCRβV-binding moiety, the second TCRβV-binding moiety, or a combination thereof comprises: (i) a VH comprising the VH sequence of a humanized Antibody B-H listed in Table 2; (ii) a VL comprising the VL sequence of a humanized Antibody B-H listed in Table 2; or (iii) a combination thereof. In some embodiments, the first TCRβV-binding moiety, the second TCRβV-binding moiety, or a combination thereof comprises: (i) the VH of a humanized Antibody B-H listed in Table 2; (ii) the VL sequence of a humanized Antibody B-H listed in Table 2; or (iii) a combination thereof.

In some embodiments, the first polypeptide chain, the second polypeptide chain, the third polypeptide chain, the fourth poly peptide chain, or a combination thereof comprises a heavy chain constant region having a sequence having at least 75%, 80%, 85%, 90%, 95%, 99%, 99.5%, 99.9%, or 100% sequence identity to any one of the sequences listed in Table 3, 21, or 22 or a combination thereof. In some embodiments, the first polypeptide chain, the second polypeptide chain, the third polypeptide chain, the fourth polypeptide chain, or a combination thereof comprises a heavy chain constant region having any one of the sequences listed in Table 3, 21, or 22 or a combination thereof. In some embodiments, the first polypeptide chain, the second polypeptide chain, the third polypeptide chain, the fourth polypeptide chain, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 3, 21, or 22 or a combination thereof. In some embodiments, the first poly peptide chain, the second polypeptide chain, the third polypeptide chain, the fourth polypeptide chain, or a combination thereof comprises a heavy chain constant region having any one of the heavy chain constant region sequences listed in Table 3, 21, or 22 or a combination thereof. In some embodiments, the first polypeptide chain, the second polypeptide chain, the third polypeptide chain, the fourth polypeptide chain, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide chain, the second polypeptide chain, the third polypeptide chain, the fourth polypeptide chain, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide chain, the second polypeptide chain, the third polypeptide chain, the fourth polypeptide chain, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide chain, the second polypeptide chain, the third polypeptide chain, the fourth poly peptide chain, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide chain, the second polypeptide chain, the third polypeptide chain, the fourth polypeptide chain, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide chain, the second polypeptide chain, the third polypeptide chain, the fourth polypeptide chain, or a combination thereof comprises a light chain constant region having a sequence having at least 75%, 80%, 85%, 90%, 95%, 99%, 99.5%, 99.9%, or 100% sequence identity to any one of the sequences listed in Table 3, 21, or 22 or a combination thereof. In some embodiments, the first polypeptide chain, the second polypeptide chain, the third polypeptide chain, the fourth polypeptide chain, or a combination thereof comprises a light chain constant region having any one of the sequences listed in Table 3, 21, or 22 or a combination thereof. In some embodiments, the first polypeptide chain, the second polypeptide chain, the third polypeptide chain, the fourth polypeptide chain, or a combination thereof comprises a light chain constant region having any one of the light chain constant region sequences listed in Table 3, 21, or 22 or a combination thereof.

In some embodiments, the first polypeptide chain, the second polypeptide chain, the third polypeptide chain, the fourth polypeptide chain, or a combination thereof comprises a light chain constant region of a kappa chain or a fragment thereof. In some embodiments, the light chain constant region of a kappa chain comprises a light chain constant region sequence listed in Table 3, 21, or 22.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13; (ii) a LC CDR1, a LC CDR2, and a LC CDR3 comprising an amino acid sequence having at least 75%, 80%, 85%, 90%, 95%, 99%. 99.5%, 99.9%, or 100% sequence identity to CDR1, CDR2, and CDR3 sequences of a VL disclosed in Tables 1, 2, 10, 11, 12 or 13; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1, CDR2, and CDR3 sequences of a VH disclosed in Tables 1, 2, 10, 11, 12 or 13; (ii) a LC CDR1, a LC CDR2, and a LC CDR3 comprising the CDR1, CDR2, and CDR3 sequences of a VL disclosed in Tables 1, 2, 10, 11, 12 or 13; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13, respectively; (ii) a LC CDR1, a LC CDR2, and a LC CDR3 comprising an amino acid sequence having at least 75%, 80%, 85%, 90%, 95%, 99%, 99.5%, 99.9%, or 100% sequence identity to CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1, CDR2, and CDR3 sequences of a VH disclosed in Tables 1, 2, 10, 11, 12 or 13, respectively; (ii) a LC CDR1, a LC CDR2, and a LC CDR3 comprising the CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 of a VH disclosed in Tables 1, 2, 10, 11, 12 or 13; (ii) a LC CDR1, a LC CDR2, and a LC CDR3 of a VL disclosed in Tables 1, 2, 10, 11, 12 or 13; or (iii) a combination thereof.

In some embodiments, the first TCRβV-binding moiety, the second TCRβV-binding moiety, or a combination thereof comprises a light chain comprising a FR1 comprising: (i) an Aspartic Acid at position 1 according to Kabat numbering; (ii) an Asparagine at position 2 according to Kabat numbering; (iii) a Leucine at position 4 according to Kabat numbering; or (iv) a combination thereof.

In some embodiments, the first TCRβV-binding moiety, the second TCRβV-binding moiety, or a combination thereof comprises a light chain comprising a FR3 comprising: (i) a Glycine at position 66 according to Kabat numbering; (ii) an Asparagine at position 69 according to Kabat numbering; (iii) a Tyrosine at position 71 according to Kabat numbering; or (iv) a combination thereof.

In some embodiments, the first TCRβV-binding moiety, the second TCRβV-binding moiety, or a combination thereof binds to an outward facing region on a TCRβV protein. In some embodiments, the outward facing region on the TCRβV protein comprises a structurally conserved region of TCRβV having a similar structure across one or more TCRβV subfamilies.

### Cytokine Molecules

In some embodiments, the multifunctional molecule includes a cytokine molecule. As used herein, a “cytokine molecule” or a “cytokine polypeptide” as interchangeably used herein, refers to full length, a fragment or a variant of a cytokine, a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis: cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD 122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 2270

[APTSSSTKKTQLQLEHLLLDLQMILNGINNYKNPKLTRMLTFKFYMPK
KATELKHLQCLEEELKPLEEVLNLAQSKNFHLRPRDLISNINVIVLELK
GSETTFMCEYADETATIVEFLNRWITFAQSIISTLT].

In another specific embodiment the IL-2 cytokine of the multispecific or multifunctional polypeptide comprises the polypeptide sequence of SEQ ID NO: 2280

[APASSSTKKTQLQLEHLLLDLQMILNGINNYKNPKLTRMLTAKFAMPK
KATELKHLQCLEEELKPLEEVLNGAQSKNFHLRPRDLISNINVIVLELK
GSETTFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 2290 [IWELKKDVYVVELDWYPDAPGEMVVLTCDTPEEDGITWTLDQSSEVLGSGKTLTIQVKEFGDAGQ YTCHKGGEVLSHSLLLLHKKEDGIWSTDILKDQKEPKNKTFLRCEAKNYSGRFTCWWLTTISTDLTF SVKSSRGSSDPQGVTCGAATLSAERVRGDNKEYEYSVECQEDSACPAAEESLPIEVMVDAVHKLKY ENYTSSFFIRDIIKPDPPKNLQLKPLKNSRQVEVSWEYPDTWSTPHSYFSLTFCVQVQGKSKREKKDR VFTDKTSATVICRKNASISVRAQDRYYSSSWSEWASVPCSGGGGSGGGGSGGGGSRNLPVATPDPG MFPCLHHSQNLLRAVSNMLQKARQTLEFYPCTSEEIDHEDITKDKTSTVEACLPLELTKNESCLNSRE TSFITNGSCLASRKTSFMMALCLSSIYEDLKMYQVEFKTMNAKLLMDPKRQIFLDQNMLAVIDELMQ ALNFNSETVPQKSSLEEPDFYKTKIKLCILLHAFRIRAVTIDRVMSYLNAS]. In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 2300

[SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGEN
LKTLRLRLRRCHRFLPCENKSKAVEQVKNAFNKLQEKGIYKAMSEFDIF
INYIEAYMTMKIRNGGGGSGGGGSGGGGSGGGGSSPGQGTQSENSCTHF
PGNLPNMLRDLRDAFSRVKTFFQMKDQLDNLLLKESLLEDFKGYLGCQA
LSEMIQFYLEEVMPQAENQDPDIKAHVNSLGENLKTLRLRLRRCHRFLP
CENKSKAVEQVKNAFNKLQEKGIYKAMSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 2310 [SPGQGTQSENSCTHFPGNLPNMLRDLRDAFSRVKTFFQMKDQLDNLLLKESLLEDFKGYLGCQALS EMIQFYLEEVMPQAENQDPDIKAHVNSLGENLKTLRLRLRRCHRFLPCENGGGSGGKSKAVEQVKN AFNKLQEKGIYKAMSEFDIFINYIEAYMTMKIRN]. In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional poly peptide according to the invention, having a long serum half-life due to the presence of an Fc domain. In some embodiments the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 2320 [NWVNVISDLKKIEDLIQSMHIDATLYTESDVHPSCKVTAMKCFLLELQVISLASGDASIHDTVENLIIL ANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence. PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-γ cytokine can elicit one or more of the cellular responses selected from the group of, increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-1a. In a specific embodiment, the MIP-1α cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-β cytokine can elicit one or more of the cellular responses selected from the group consisting of, chemotaxis in monocytes, chemotaxis in macrophages, upregulation of IL-1 expression in activated macrophages, and upregulation of IgA expression in activated B cells.

In some embodiments, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine: a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIHDTVENLIIL ANNSLSSNGNVTESGCKECEELEEKNIKEFLQSFVHIVQMFINTS (SEQ ID NO: 2170), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2170.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYICNSGFKRKAG TSSLTECVL (SEQ ID NO: 2180), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2180. In some embodiments, the cytokine molecule (e.g. IL-15) and the receptor dimerizing domain (e.g., an IL. 15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 2190). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCLEEELKPLE EVLNLAQSKNFHLRPRDLISNINVIVLELKGSETTFMCEYADETATIVEFLNRWITFCQSIISTLT (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGMAVTISVKC EKISTLSCENKIISFKEMNPPDNIKDTKSDIIFFQRSVPGHDNKMQFESSSYEGYFLACEKERDLFKLIL KKEDELGDRSIMFTVQNED (SEQ ID NO: 2192), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2192).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSANTGNNERII NVSIKKLKRKPPSTNAGRRQKHRLTCPSCDSYEKKPPKEFLERFKSLLQKMIHQHLSSRTHGSEDS (SEQ ID NO: 2193), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2193).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFKNFKDDQSIQ KSVETIKEDMNVKFFNSNKKKRDDFEKLTNYSVTDLNVQRKAIHELIQVMAELSPAAKTGKRKRSQ MLFRG (SEQ ID NO: 2194), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2194).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers:

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells: this receptor plays a major role in triggering cytotoxicity against target cells expressing cell surface ligands such as MHC class I chain-related MICA and MICB, and U (optionally L1) 6-binding proteins (ULBPs): it KLRK1-HCST receptor plays a role in immune surveillance against tumors and is required for cytolysis of tumors cells: indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

Provided herein are, inter alia, multispecific (e.g., bi-, tri-, quad-specific) or multifunctional molecules that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGDHQEAFRPG AIVSPWRLKSGDASLRLPGIQLEEAGEYRCEVVVTPLKAQGTVQLEVVASPASRLLLDQVGMKENE DKYMCESSGFYPEAINITWEKQTQKFPHPIEISEDVITGPTIKNMDGTFNVTSCLKLNSSQEDPGTVYQ CVVRHASLHTPLRSNFTLTAARHSLSETEKTDNFS (SEQ ID NO: 3291), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3291.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September: 31 (9): 2680-9 “Recognition of viral hemagglutinins by NKp44 but not by NKp30”; and Nature. 2001 Feb. 22; 409 (6823): 1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein: (i) MICA comprises the amino acid sequence: EPHSLRYNLTVLSWDGSVQSGFLTEVHLDGQPFLRCDRQKCRAKPQGQWAEDVLGNKTWDRETRD LTGNGKDLRMTLAHIKDQKEGLHSLQEIRVCEIHEDNSTRSSQHFYYDGELFLSQNLETKEWTMPQS SRAQTLAMNVRNFLKEDAMKTKTHYHAMHADCLQELRRYLKSGVVLRRTVPPMVNVTRSEASEG NITVTCRASGFYPWNITLSWRQDGVSLSHDTQQWGDVLPDGNGTYQTWVATRICQGEEQRFTCYM EHSGNHSTHPVPSGKVLVLQSHW (SEQ ID NO: 3292), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3292; (ii) MICB comprises the amino acid sequence: AEPHSLRYNLMVLSQDESVQSGFLAEGHLDGQPFLRYDRQKRRAKPQGQWAEDVLGAKTWDTETE DLTENGQDLRRTLTHIKDQKGGLHSLQEIRVCEIHEDSSTRGSRHFYYDGELFLSQNLETQESTVPQSS RAQTLAMNVTNFWKEDAMKTKTHYRAMQADCLQKLQRYLKSGVAIRRTVPPMVNVTCSEVSEGN ITVTCRASSFYPRNITLTWRQDGVSLSHNTQQWGDVLPDGNGTYQTWVATRIRQGEEQRFTCYMEH SGNHGTHPVPSGKVLVLQSQRTD (SEQ ID NO: 3293), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3293; or (iii) ULBP1 comprises the amino acid sequence: GWVDTHCLCYDFIITPKSRPEPQWCEVQGLVDERPFLHYDCVNHKAKAFASLGKKVNVTKTWEEQT ETLRDVVDFLKGQLLDIQVENLIPIEPLTLQARMSCEHEAHGHGRGSWQFLFNGQKFLLFDSNNRKW TALHPGAKKMTEKWEKNRDVTMFFQKISLGDCKMWLEEFLMYWEQMLDPTKPPSLAPG (SEQ ID NO: 3294), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3294.

In other embodiments, the NK cell engager is a ligand of DNAM1 chosen from NECTIN2 or NECL5, e.g., wherein: (i) NECTIN2 comprises the amino acid sequence: QDVRVQVLPEVRGQLGGTVELPCHLLPPVPGLYISLVTWQRPDAPANHQNVAAFHPKMGPSFPSPKP GSERLSFVSAKQSTGQDTEAELQDATLALHGLTVEDEGNYTCEFATFPKGSVRGMTWLRVIAKPKN QAEAQKVTFSQDPTTVALCISKEGRPPARISWLSSLDWEAKETQVSGTLAGTVTVTSRFTLVPSGRAD GVTVTCKVEHESFEEPALIPVTLSVRYPPEVSISGYDDNWYLGRTDATLSCDVRSNPEPTGYDWSTTS GTFPTSAVAQGSQLVIHAVDSLFNTTFVCTVTNAVGMGRAEQVIFVRETPNTAGAGATGG (SEQ ID NO: 3295), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3295; or (ii) NECL5 comprises the amino acid sequence: WPPPGTGDVVVQAPTQVPGFLGDSVTLPCYLQVPNMEVTHVSQLTWARHGESGSMAVFHQTQGPS YSESKRLEFVAARLGAELRNASLRMFGLRVEDEGNYTCLFVTFPQGSRSVDIWLRVLAKPQNTAEV QKVQLTGEPVPMARCVSTGGRPPAQITWHSDLGGMPNTSQVPGFLSGTVTVTSLWILVPSSQVDGK NVTCKVEHESFEKPQLLTVNLTVYYPPEVSISGYDNNWYLGQNEATLTCDARSNPEPTGYNWSTTM GPLPPFAVAQGAQLLIRPVDKPINTTLICNVTNALGARQAELTVQVKEGPPSEHSGISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD 16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSSELKVSLTN VSISDEGRYFCQLYTDPPQESYTTITVLVPPRNLMIDIQKDTAVEGEEIEVNCTAMASKPATTIRWFKG NTELKGKSEVEEWSDMYTVTSQLMLKVHKEDDGVPVICQVEHPAVTGNLQTQRYLEVQYKPQVHI QMTYPLQGLTREGDALELTCEAIGKPQPVMVTWVRVDDEMPQHAVLSGPNLFINNLNKTDNGTYR CEASNIVGKAHSDYMLYVYDPPTTIPPPTTTTTTTTTTTTTILTIITDSRAGEEGSIRAVDH (SEQ ID NO: 3297), a fragment thereof, or an amino acid sequence substantially identical thereto (e.g. 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3297.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRDGIY MVHIQVTLAICSSTTASRHHPTTLAVGICSPASRSISLLRLSFHQGCTIASQRLTPLARGDTLCTNLTGT LLPSRNTDETFFGVQWVRP (SEQ ID NO: 3298), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3298.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWVGTN KSLTEEAENWGDGEPNNKKNKEDCVEIYIKRNKDAGKWNDDACHKLKAALCYTASCQPWSCSGH GECVEIINNYTCNCDVGYYGPQCQFVIQCEPLEAPELGTMDCTHPLGNFSFSSQCAFSCSEGTNLTGIE ETTCGPFGNWSSPEPTCQVIQCEPLSAPDLGIMNCSHPLASFSFTSACTFICSEGTELIGKKKTICESSGI WSNPSPICQKLDKSFSMIKEGDYN (SEQ ID NO: 3299), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3299.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAVFHQTQGPS YSESKRLEFVAARLGAELRNASLRMFGLRVEDEGNYTCLFVTFPQGSRSVDIWLRVLAKPQNTAEV QKVQLTGEPVPMARCVSTGGRPPAQITWHSDLGGMPNTSQVPGFLSGTVTVTSLWILVPSSQVDGK NVTCKVEHESFEKPQLLTVNLTVYYPPEVSISGYDNNWYLGQNEATLTCDARSNPEPTGYNWSTTM GPLPPFAVAQGAQLLIRPVDKPINTTLICNVTNALGARQAELTVQVKEGPPSEHSGISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQRAHQAAE GQLQACQADRQKTKETLQSEEQQRRALEQKLSNMENRLKPFFTCGSADTCCPSGWIMHQKSCFYISL TSKNWQESQKQCETLSSKLATFSEIYPQSHSYYFLNSLLPNGGSGNSYWTGLSSNKDWKLTDDTQRT RTYAQSSKCNKVHKTWSWWTLESESCRSSLPYICEMTAFRFPD (SEQ ID NO: 3300), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3300.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSSDHW IGLKMAKNRTGQWVDGATFTKSFGMRGSEGCAYLSDDGAATARCYTERKWICRKRIH (SEQ ID NO: 3301), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3301.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQSG ALYISKVQKEDNSTYIMRVLKKTGNEQEWKIKLQVLDPVPKPVIKIEKIEDMDDNCYLKLSCVIPGES VNYTWYGDKRPFPKELQNSVLETTLMPHNYSRCYTCQVSNSVSSKNGTVCLSPPCTLARS (SEQ ID NO: 3302), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3302.

### T Cell Engagers

Provided herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRβ, e.g., a TCRβV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

Provided herein are, inter alia, multispecific (e.g., bi-, tri-, quad-specific) or multifunctional molecules that further include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist. TLR4 (e.g., caTLR4 (constitutively active TLR4)). CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L): a Toll-like receptor agonist (e.g., a TLR4, e.g., a constitutively active TLR4 (caTLR4) or a TLR9 agonist); a 41BB agonist: a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist: a CD40L; an OX40L; an antibody molecule that binds to OX40, CD40 or CD70; a Toll-like receptor agonist or a fragment thereof (e.g., a TLR4, e.g., a constitutively active TLR4 (caTLR4)): a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4. e.g., a constitutively active TLR4 (caTLR4)), CD47 agonist, or a STING agonist.

In some embodiments, the OX40L comprises the amino acid sequence: QVSHRYPRIQSIKVQFTEYKKEKGFILTSQKEDEIMKVQNNSVIINCDGFYLISLKGYFSQEVNISLHY QKDEEPLFQLKKVRSVNSLMVASLTYKDKVYLNVTTDNTSLDDFHVNGGELILIHQNPGEFCVL (SEQ ID NO: 3303), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3303.

In another embodiment, the CD40L comprises the amino acid sequence: MQKGDQNPQIAAHVISEASSKTTSVLQWAEKGYYTMSNNLVTLENGKQLTVKRQGLYYIYAQVTF CSNREASSQAPFIASLCLKSPGRFERILLRAANTHSSAKPCGQQSIHLGGVFELQPGASVFVNVTDPSQ VSHGTGFTSFGLLKL (SEQ ID NO: 3304), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3304.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In some embodiments, the immune cell engager includes 41BB ligand, e.g., comprising the amino acid sequence: ACPWAVSGARASPGSAASPRLREGPELSPDDPAGLLDLRQGMFAQLVAQNVLLIDGPLSWYSDPGL AGVSLTGGLSYKEDTKELVVAKAGVYYVFFQLELRRVVAGEGSGSVSLALHLQPLRSAAGAAALAL TVDLPPASSEARNSAFGFQGRLLHLSAGQRLGVHLHTEARARHAWQLTQGATVLGLFRVTPEIPAGL PSPRSE (SEQ ID NO: 3305), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3305.

Toll-Like Receptors: Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63).

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a My D88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O, et al., 1998. Targeted disruption of the MyD88 gene results in loss of IL-1- and IL-18-mediated function. Immunity. 9 (1): 143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S, et al. 2002. IRF3 mediates a TLR3/TLR4-specific antiviral gene program. Immunity 17 (3): 251-63) Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDC's through mTOR. Nature Immunol. 9:1097-1099).

TLR-9: TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-1 interferon and IL-12.

TLR Agonists: a TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate. In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides. In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3″ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Stromal Modifying Moieties

In some embodiments, the multifunctional molecule further includes a stromal modifying moiety. A “stromal modifying moiety.” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g. collagen, laminin, elastin, fibrinogen, fibronectin, and vitronectin.

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton. B C Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101:805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates: (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products: (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stern, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations J Carcinog Mutage 5:178; U.S. Pat. Nos. 7,767,429; 8,202,517; 7,431,380; 8,450,470; 8,772,246; 8,580,252, the entire contents of each of which is incorporated by reference herein), rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 3306)
LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 139, such as 36-481, 36-482, 36-483, where the full-length PH20 has the sequence of amino acids set forth in SEQ ID NO: 139; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 139; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94%, 95%, 96%, 97%, 98%, 99% or more sequence identity with the polypeptide set forth in SEQ ID NO: 139 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947. T. Yamagata et al. J. Biol. Chem., 243, 1523 (1968). S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada. J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp 102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich. Biochem. J., 151, 121 (1975), Kenichi Maeyama et al, Seikagaku, 57, 1189 (1985)), chondroitinase C (derived from Flavobacterium sp. Hp102; Hirofumi Miyazono et al. Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15, -15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases. TIMPs, as well as by non-specific proteinase inhibitors (Ala-aho R et al. Biochimie. Collagenases in cancer. 2005 March-April: 87 (3-4): 273-86) In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119; doi:10.1046/j. 1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component: decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a functional fragment or a functional variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDRLGYYPYIDSI TGVTVNGGIPQKISLQDHLDKAKKDITFYMPVDNLGMAVIDWEEWRPTWARNWKPKDVYKNRSIE LVQQQNVQLSLTEATEKAKQEFEKAGKDFLVETIKLGKLLRPNHLWGYYLFPDCYNHHYKKPGYN GSCFNVEIKRNDDLSWLWNESTALYPSIYLNTQQSPVAATLYVRNRVREAIRVSKIPDAKSPLPVFAY TRIVFTDQVLKFLSQDELVYTFGETVALGASGIVIWGTLSIMRSMKSCLLLDNYMETILNPYIINVTLA AKMCSQVLCQEQGVCIRKNWNSSDYLHLNPDNFAIQLEKGGKFTVRGKPTLEDLEQFSEKFYCSCYS TLSCKEKADVKDTDAVDVCIADGVCIDAFLKPPMETEEPQIFYNASPSTLS (SEQ ID NO:3311), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3311.

In some embodiments, the hyaluronidase molecule comprises: (i) the amino acid sequence of 36-464 of SEQ ID NO: 3311; (ii) the amino acid sequence of 36-481, 36-482, or 36-483 of PH20, wherein PH20 has the sequence of amino acids set forth in SEQ ID NO: 3311; or (iii) an amino acid sequence having at least 95% to 100% sequence identity to the polypeptide or truncated form of sequence of amino acids set forth in SEQ ID NO: 3311; or (iv) an amino acid sequence having 30, 20, 10, 5 or fewer amino acid substitutions to the amino acid sequence set forth in SEQ ID NO: 3311. In some embodiments, the hyaluronidase molecule comprises an amino acid sequence at least 95% (e.g., at least 95%, 96%, 97%, 98%, 99%, 100%) identical to the amino acid sequence of SEQ ID NO: 3311. In some embodiments, the hyaluronidase molecule is encoded by a nucleotide sequence at least 95% (e.g., at least 96%, 97%, 98%, 99%, 100%) identical to the nucleotide sequence of SEQ ID NO: 3311.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQGTYPYYTPT GEPVFGGLPQNASLIAHLARTFQDILAAIPAPDFSGLAVIDWEAWRPRWAFNWDTKDIYRQRSRALV QAQHPDWPAPQVEAVAQDQFQGAARAWMAGTLQLGRALRPRGLWGFYGFPDCYNYDFLSPNYTG QCPSGIRAQNDQLGWLWGQSRALYPSIYMPAVLEGTGKSQMYVQHRVAEAFRVAVAAGDPNLPVL PYVQIFYDTTNHFLPLDELEHSLGESAAQGAAGVVLWVSWENTRTKESCQAIKEYMDTTLGPFILNV TSGALLCSQALCSGHGRCVRRTSHPKALLLLNPASFSIQLTPGGGPLSLRGALSLEDQAQMAVEFKCR CYPGWQAPWCERKSMW (SEQ ID NO: 3312), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3312.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADIMINFGRWE HGDGYPFDGKDGLLAHAFAPGTGVGGDSHFDDDELWTLGEGQVVRVKYGNADGEYCKFPFLFNG KEYNSCTDTGRSDGFLWCSTTYNFEKDGKYGFCPHEALFTMGGNAEGQPCKFPFRFQGTSYDSCTT EGRTDGYRWCGTTEDYDRDKKYGFCPETAMSTVGGNSEGAPCVFPFTFLGNKYESCTSAGRSDGK MWCATTANYDDDRKWGFCPDQGYSLFLVAAHEFGHAMGLEHSQDPGALMAPIYTYTKNFRLSQD DIKGIQELYGASPDIDLGTGPTPTLGPVTPEICKQDIVFDGIAQIRGEIFFFKDRFIWRTVTPRDKPMGPL LVATFWPELPEKIDAVYEAPQEEKAVFFAGNEYWIYSASTLERGYPKPLTSLGLPPDVQRVDAAFNW SKNKKTYIFAGDKFWRYNEVKKKMDPGFPKLIADAWNAIPDNLDAVVDLQGGGHSYFFKGAYYLK LENQSLKSVKFGSIKSDWLGC (SEQ ID NO: 3313), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3313.

### Tumor Antigen Moiety

In some embodiments, the multifunctional molecule further includes a tumor antigen moiety. In some embodiments, the tumor-associated antigen binding moiety is an antigen, e.g., a cancer antigen. In some embodiments, the cancer antigen is a tumor antigen or stromal antigen, or a hematological antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors. As used herein, the terms “tumor antigen”, “tumor-associated antigen”, and “tumor associate antigen” can be used interchangeably. As used herein, the terms “tumor-targeting moiety” and “the tumor-associated antigen binding moiety” can be used interchangeably.

In some embodiments, the tumor-associated antigen binding moiety, e.g., cancer antigen, is chosen from: BCMA, FcRH5, CD19, CD20, CD22, CD30, CD33, CD38. CD47, CD99, CD 123, FcRH5, CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP 1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Integrins (Integrin alphaVbeta3, Integrin alpha5Beta1), Carbohydrates (Le), IGF1R, EPHA3, TRAILR1, TRAILR2, RANKL, (FAP), TGF-beta, hyaluronic acid, collagen, e.g., collagen IV, tenascin C, or tenascin W. In some embodiments, the tumor-associated antigen binding moiety, e.g., cancer antigen, is BCMA. In some embodiments, the tumor-associated antigen binding moiety, e.g., cancer antigen, is FcRH5.

In some embodiments, the tumor-associated antigen binding moiety binds to a cancer antigen chosen from: CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38. CD44v6. Carcinoembryonic antigen (CEA), Epithelial cell adhesion molecule (EPCAM), B7H3 (CD276). KIT (CD117). Interleukin-13 receptor subunit alpha-2, mesothelin. Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit. Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3). G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP). Wilms tumor protein (WT1), Cancer/testis antigen 1 (NY-ESO-1). Cancer/testis antigen 2 (LAGE-1a), Melanoma-associated antigen 1 (MAGE-A1). ETS translocation-variant gene 6, located on chromosome 12p (ETV6-AML), sperm protein 17 (SPA 17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA 17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2). Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma. X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2). CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

### FcRH5 Targeting Moieties:

In some embodiments, the multispecific molecules as described herein include a targeting moiety that binds to FcRH5 (e.g., a FcRH5 targeting moiety). The FcRH5 targeting moiety can be chosen from an antibody molecule (e.g., an antigen binding domain as described herein), a receptor or a receptor fragment, or a ligand or a ligand fragment, or a combination thereof. In some embodiments, the FcRH5 targeting moiety associates with, e.g., binds to, a cancer or hematopoietic cell (e.g., a molecule, e.g., antigen, present on the surface of the cancer or hematopoietic cell). In certain embodiments, the FcRH5 targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the FcRH5 targeting moiety, binds to a FcRH5 antigen on the surface of a cell, e.g., a cancer or hematopoietic cell. The FcRH5 antigen can be present on a primary tumor cell, or a metastatic lesion thereof. In some embodiments, the cancer is a hematological cancer, e.g., multiple myeloma. For example, the FcRH5 antigen can be present on a tumor, e.g., a tumor of a class typified by having one or more of: limited tumor perfusion, compressed blood vessels, or fibrotic tumor interstitium.

The multispecific molecules described herein includes a FcRH5 targeting moiety that comprises an anti-FcRH5 antibody or antigen-binding fragment thereof described in U.S. Pat. No. 7,999,077, US20150098900, U.S. Pat. No. 8,299,220. U.S. Pat. Nos. 7,105,149, 8,362,213, 8,466,260. U.S. Pat. No. 8,617,559, US20160368985, US20150166661, and US20080247944, the entire contents of any of the aforesaid publications are herein incorporated by reference.

In some embodiments, the multispecific molecules described herein includes a FcRH5 targeting moiety that comprises an anti-FcRH5 antibody or antigen-binding fragment thereof described in U.S. Pat. No. 7,999,077, the entire contents of which are herein incorporated by reference.

### BCMA Targeting Moieties:

In certain embodiments, the multispecific molecules as described herein include a targeting moiety that binds to BCMA (e.g., a BCMA targeting moiety). The BCMA targeting moiety can be chosen from an antibody molecule (e.g., an antigen binding domain as described herein), a receptor or a receptor fragment, or a ligand or a ligand fragment, or a combination thereof. In some embodiments, the BCMA targeting moiety associates with, e.g., binds to, a cancer or hematopoietic cell (e.g., a molecule, e.g., antigen, present on the surface of the cancer or hematopoietic cell). In certain embodiments, the BCMA targeting moiety targets, e.g. directs the multispecific molecules as described herein to a cancer or hematopoietic cell. In some embodiments, the cancer is a hematological cancer, e.g., multiple myeloma.

In some embodiments, the multispecific molecule, e.g., the BCMA targeting moiety, binds to a BCMA antigen on the surface of a cell, e.g., a cancer or hematopoietic cell. The BCMA antigen can be present on a primary tumor cell, or a metastatic lesion thereof. In some embodiments, the cancer is a hematological cancer, e.g., multiple myeloma. For example, the BCMA antigen can be present on a tumor, e.g., a tumor of a class typified by having one or more of: limited tumor perfusion, compressed blood vessels, or fibrotic tumor interstitium.

Exemplary BCMA targeting moieties: the multispecific molecules described herein can include a BCMA targeting moiety that comprises an anti-BCMA antibody or antigen-binding fragment thereof described in U.S. Pat. Nos. 8,920,776, 9,243,058, 9,340,621, 8,846,042, 7,083,785, 9,545,086, 7,276,241, 9,034,324, 7,799,902, 9,387,237. U.S. Pat. No. 8,821,883. U.S. Pat. No. 861,745, US20130273055, US20160176973. US20150368351, US20150376287, US20170022284, US20160015749, US20140242077, US20170037128, US20170051068, US20160368988, US20160311915, US20160131654, US20120213768, US20110177093, US20160297885, EP3137500, EP2699259, EP2982694, EP3029068, EP3023437, WO2016090327, WO2017021450, WO2016110584, WO2016118641, WO2016168149, the entire contents of which are incorporated herein by reference.

In some embodiments, the BCMA-targeting moiety includes an antibody molecule (e.g., Fab or scFv) that binds to BCMA. In some embodiments, the antibody molecule to BCMA comprises one, two, or three CDRs from any of the heavy chain variable domain sequences of Table 1, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a heavy chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

Alternatively, or in combination with the heavy chain to BCMA as described herein, the antibody molecule to BCMA comprises one, two, or three CDRs from any of the light chain variable domain sequences of Table 15, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a light chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells) In some embodiments, the cancer is chosen from a hematological cancer, a solid cancer, a metastatic cancer, or a combination thereof.

In some embodiments, the multispecific molecule, e.g., the tumor-associated antigen binding moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, ganglioside 2 (GD2), prostate stem cell antigen (PSCA), prostate specific membrane antigen (PMSA), prostate-specific antigen (PSA), carcinoembryonic antigen (CEA), Ron Kinase, c-Met. Immature laminin receptor. TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17. Tyrosinase, TRP-1/-2, MC1R, β-catenin, BRCA1/2, CDK4, CML66, Fibronectin, p53. Ras, TGF-B receptor. AFP. ETA. MAGE, MUC-1, CA-125, BAGE, GAGE, NY-ESO-1, β-catenin, CDK4, CDC27, CD47, a actinin-4, TRP1/gp75, TRP2, gp100, Melan-A/MARTI, gangliosides, WT1, EphA3. Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33. GD3. GM2. VEGFR, Integrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-associated antigen binding moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma. Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma. Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA. CD19, CD20, CD22, CD33. CD123, FcRH5, CLEC12, or CD179A.

In some embodiments, the tumor-associated antigen-binding moiety comprises an antibody or an antigen-binding moiety thereof that binds to CD19, CD123, CD22, CD30, CD171, CS-1, C-type lectin-like molecule-1. CD33, CISH, epidermal growth factor receptor variant III (EGFRvIII), ganglioside G2 (GD2), ganglioside GD3, DLL3, CLDN18.2. TNF receptor family member B cell maturation (BCMA), In antigen, prostate-specific membrane antigen (PSMA). Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin. Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta). Stage-specific embryonic antigen-4 (SSEA-4). CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2). Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2). Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97. CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2). Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP). Wilms tumor protein (WT1). Cancer/testis antigen 1 (NY-ESO-1), Cancer/testis antigen 2 (LAGE-1a), Melanoma-associated antigen 1 (MAGE-A1). ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant prostein, survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene). N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK). A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a. CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF). C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1) antigen.

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian. e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule. e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g. IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883). (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain: such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat. E. A., et al. (1991) Sequences of Proteins of Immunological Interest, Fifth Edition, U.S. Department of Health and Human Services. NIH Publication No. 91-3242; Chothia, C, et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg; or by ImMunoGene Tics (IMGT) numbering system).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273.927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO) J 12:725-734; Hawkins et al. (1992). J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg. N, et al. 1994 Nature 368:856-859; Green. L. L, et al. 1994 Nature Genet. 7:13-21; Morrison, S. L, et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32: 180-190 (1990)) and also because of potential allergic reactions (see, e.g., LoBuglio et al., Hybridoma. 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi. M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043): Liu et al. (1987) PNAS 84.3439-3443; Liu et al., 1987, J. Immunol. 139:3521-3526; Sun et al. (1987) PNAS 84:214-218; Nishimura et al. 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker. From Genes to Clones (Verlagsgesellschaft. Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229: 1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoevan et al. 1988 Science 239:1534; Beidler et al. 1988 J. Immunol. 141:4053-4060. Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D, et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g. the heavy chain constant regions of IgG1, IgG2, IgG3, IgG4. IgM, IgA1, IgA2. IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not: recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g., altered affinity for an effector ligand, such as FcR on a cell, or the C1 component of complement can be produced by replacing at least one amino acid residue in the constant portion of the antibody with a different residue (see e.g., EP 388.151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR 1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess. 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors: MHC proteins; extracellular domains (e.g., fibronectin Type Ill repeats, EGF repeats); protease inhibitors (e.g., Kunitz domains, ecotin, BPTI, and so forth): TPR repeats: trifoil structures: zinc finger domains: DNA-binding proteins: particularly monomeric DNA binding proteins: RNA binding proteins: enzymes, e.g., proteases (particularly inactivated proteases), RNase: chaperones, e.g., thioredoxin, and heat shock proteins; and intracellular signaling domains (such as SH2 and SH3 domains). See, e.g., US20040009530 and U.S. Pat. No. 7,501,121, incorporated herein by reference.

In some embodiments, a scaffold domain is evaluated and chosen, e.g., by one or more of the following criteria: (1) amino acid sequence. (2) sequences of several homologous domains. (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma. J, et al. (1997) Nature Biotech 15:159.

Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J, et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. No. 9,145,588B. U.S. Pat. No. 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670. U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S. L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 14.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain) Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J, et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g. T366Y or T366W): “Holes” or “cavities” are created by replacing a larger residue in a parental antibody with a smaller amino acid (e.g., Y407T. T366S, L368A and/or Y407V).

For bispecific antibodies including an Fc domain, introduction of specific mutations into the constant region of the heavy chains to promote the correct heterodimerization of the Fc portion can be utilized Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A. Y407V in the “hole” heavy chain. Other exemplary KiH mutations are provided in Table 4, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006:12:6144-52; PMID: 17062691). Xencor defined 41 variant pairs based on combining structural calculations and sequence information that were subsequently screened for maximal beterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C (H) 3 heterodimers are known. These derivatives of human IgG and IgA C (H) 3 domains create complementary human SEED C (H) 3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID: 20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110 (13): 5145-5150 and Labrijn et al. Nature Protocols 2014; 9 (10): 2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133. U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4 (28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001. US20140072581A1, US20160039947A1, and US20150368352.

### Lambda Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017, and designated publication number WO 2018/057955, incorporated herein by reference in its entirety.

In some embodiments, the multispecific molecule includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1, LLCP1, together with its HCP1, provide specificity for a first epitope (while KLCP2, together with its HCP2, provide specificity for a second epitope). As described elsewhere herein, LLCP1 has a higher affinity for HCP 1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1, HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2. (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide molecule as described herein:
LLCP1 has a higher affinity for HCP1 than for HCP2; and/or
KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments, in the multifunctional polypeptide molecule as described herein:
the HCP1 has a greater affinity for HCP2, than for a second molecule of HCP1; and/or
the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, described herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments. (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda- and/or kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In some embodiments, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes: (i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope:
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid VLλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant. CH2-CH3 domain (having a bole modification).

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10:1-18 (2013); and Spiess C et al. (2015) Alternative molecular formats and therapeutic applications for bispecific antibodies. Molecular Immmology 67:95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67 (2015). 95-106. Exemplary BsIgGs include catumaxomab (Fresenius Biotech. Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67 (2015): 95-106. Strategies that have been used to avoid heavy chain pairing of homodimers in BsIgG include knobs-in-boles, duobody, asymmetric, charge pair, HA-TF. SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H) IgG, IgG(L)-scFv, scFv-(L) IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67 (2015): 95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS. BITE, Diabody. DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Exemplary Bispecific Molecules

In an aspect, a multispecific molecule as described herein comprises a sequence as described herein. e.g., a sequence chosen from SEQ ID NOs: 1004-1007, 3275-3277, 3286, or 3287, or a sequence with at least 85%, 90%, 95%, 96%, 97%, 98%, 99% or more identity thereto. In some embodiments, a multispecific molecule as described herein comprises a leader sequence comprising the amino acid sequence of SEQ ID NO: 3288. In some embodiments, a multispecific molecule as described herein does not comprise a leader sequence comprising the amino acid sequence of SEQ ID NO: 3288.

Molecule F: aCD19×aVb6.5: Molecule F comprises a heavy chain comprising the amino acid sequence of SEQ ID NO: 1004 and a light chain comprising the amino acid sequence of SEQ ID NO: 1005

Molecule F.1
(heavy chain) (Torvbeta6_5 scFv/anti-CD19 heavy chain)
SEQ ID NO: 1004
METDTLLLWVLLLWVPGSTGQVQLVQSGAEVKKPGSSVKVSCKASGYSFTTYYIHWVRQAPGQGL
EWMGWFFPGSGNIKYNEKFKGRVTITADTSTSTAYMELSSLRSEDTAVYYCAGSYYSYDVLDYWG
QGTTVTVSSGGGGSGGGGSGGGGSGGGGSDIQMTQSPSFLSASVGDRVTITCKASQNVGINVVWHQ
QKPGKAPKALIYSSSHRYSGVPSRFSGSGSGTEFTLTISSLQPEDFATYFCQQFKSYPLTFGQGTKLEIK
GGGGSQVTLRESGPALVKPTQTLTLTCTFSGFSLSTSGMGVGWIRQPPGKALEWLAHIWWDDDKRY
NPALKSRLTISKDTSKNQVFLTMTNMDPVDTATYYCARMELWSYYFDYWGQGTTVTVSSASTKGP
SVFPLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
Molecule F.2
(light chain) (anti-CD19 light chain)
SEQ ID NO: 1005
METPAQLLFLLLLWLPDTTGENVLTQSPATLSLSPGERATLSCSASSSVSYMHWYQQKPGQAPRLLI
YDTSKLASGIPARFSGSGSGTDHTLTISSLEPEDFAVYYCFQGSVYPFTFGQGTKLEIKRTVAAPSVFIF
PPSDEQLKSGTASVVCLLNNFYPREAKVQWKVDNALQSGNSQESVTEQDSKDSTYSLSSTLTLSKAD
YEKHKVYACEVTHQGLSSPVTKSFNRGEC

In an aspect, a multispecific molecule as described herein comprises SEQ ID NO: 1004 and/or SEQ ID NO: 1005 or a sequence with at least 85%, 90%, 95%, 96%, 97%, 98%, 99% or more identity thereto.

Molecule G: aBCMA×aVb6.5: Molecule G comprises a heavy chain comprising the amino acid sequence of SEQ ID NO: 1006 and a light chain comprising the amino acid sequence of SEQ ID NO: 1007.

Molecule G.1
(heavy chain)
SEQ ID NO: 1006
METDTLLLWVLLLWVPGSTGQVQLVQSGAEVKKPGSSVKVSCKASGYSFTTYYIHWVRQAPGQGL
EWMGWFFPGSGNIKYNEKFKGRVTITADTSTSTAYMELSSLRSEDTAVYYCAGSYYSYDVLDYWG
QGTTVTVSSGGGGSGGGGSGGGGSGGGGSDIQMTQSPSFLSASVGDRVTITCKASQNVGINVVWHQ
QKPGKAPKALIYSSSHRYSGVPSRFSGSGSGTEFTLTISSLQPEDFATYFCQQFKSYPLTFGQGTKLEIK
GGGGSQVQLVESGGGVVQPGRSLRLSCAASGIDFSRYWMSWVRQAPGKGLEWVGEINPDSSTINYA
PSLKDRFTISRDNSKNTLYLQMSSLRAEDTAVYYCASLYYDYGDAMDYWGQGTTVTVSSASTKGPS
VFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNRFTQKSLSLSPGK
Molecule G.2
(light chain)
SEQ ID NO: 1007
METDTLLLWVLLLWVPGSTGDIQMTQSPSSLSASVGDRVTITCKASQSVDSNVAWYQQKPEKAPKA
LIFSASLRFSGVPSRFSGSGSGTDFTLTISSLQPEDFATYFCQQYNNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC

In an aspect, a multispecific molecule as described herein comprises SEQ ID NO: 1006 and/or SEQ ID NO: 1007 or a sequence with at least 85%, 90%, 95%, 96%, 97%, 98%, 99% or more identity thereto.

Molecule H: aBCMA×aTCRvbeta6_5: Molecule H comprises a first heavy chain comprising the amino acid sequence of SEQ ID NO: 3275, a light chain comprising the amino acid sequence of SEQ ID NO: 3277, and a second heavy chain comprising the amino acid sequence of SEQ ID NO: 3276.

Molecule H.1
(anti-BCMA heavy chain)
SEQ ID NO: 3275
METDTLLLWVLLLWVPGSTGQVQLVESGGGVVQPGRSLRLSCAASGIDFSRYWMSWVRQAPGKGL
EWVGEINPDSSTINYAPSLKDRFTISRDNSKNTLYLQMSSLRAEDTAVYYCASLYYDYGDAMDYWG
QGTTVTVSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNATYRVVSVLTVLH
QDWLNGKEYKCKVSNKALPAPIEKTISKAKGQPREPQVYTLPPCREEMTKNQVSLWCLVKGFYPSDI
AVEWESNGQPENNYKTTPPVLDSDGSFFLYSKLTVDKSRWQQGNVFSCSVMHEALHNHYTQKSLSL
SPGK
Molecule H.2
(TCRvbeta_6_5 scFv humanized)
SEQ ID NO: 3276
METDTLLLWVLLLWVPGSTGQVQLVQSGAEVKKPGSSVKVSCKASGYSFTTYYIHWVRQAPGQGL
EWMGWFFPGSGNIKYNEKFKGRVTITADTSTSTAYMELSSLRSEDTAVYYCAGSYYSYDVLDYWG
QGTTVTVSSGGGGSGGGGSGGGGSGGGGSDIQMTQSPSFLSASVGDRVTITCKASQNVGINVVWHQ
QKPGKAPKALIYSSSHRYSGVPSRFSGSGSGTEFTLTISSLQPEDFATYFCQQFKSYPLTFGQGTKLEIK
GGGGSGGGGSDKTHTCPPCPAPELLGGPSVFLFPPKPKDTLMISRTPEVTCVVVDVSHEDPEVKFNW
YVDGVEVHNAKTKPREEQYASTYRVVSVLTVLHQDWLNGKEYKCKVSNKALPAPIEKTISKAKGQP
REPQVCTLPPSREEMTKNQVSLSCAVKGFYPSDIAVEWESNGQPENNYKTTPPVLDSDGSFFLVSKLT
VDKSRWQQGNVFSCSVMHEALHNHYTQKSLSLSPGK
Molecule H.3
(anti-BCMA light chain)
SEQ ID NO: 3277
METDTLLLWVLLLWVPGSTGDIQMTQSPSSLSASVGDRVTITCKASQSVDSNVAWYQQKPEKAPKA
LIFSASLRFSGVPSRFSGSGSGTDFTLTISSLQPEDFATYFCQQYNNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC

In an aspect, a multispecific molecule as described herein comprises SEQ ID NO: 3275, SEQ ID NO: 3276, and/or SEQ ID NO: 3277 or a sequence with at least 85%, 90%, 95%, 96%, 97%, 98%, 99% or more identity thereto.

Molecule I: half arm BCMA Fab with c-terminal scFv TCRvbeta: Molecule I comprises a first heavy chain comprising the amino acid sequence of SEQ ID NO: 3286, a light chain comprising the amino acid sequence of SEQ ID NO: 3277, and a second heavy chain comprising the amino acid sequence of SEQ ID NO: 3287.

Molecule I.1
(heavy chain 1)
SEQ ID NO: 3286
METDTLLLWVLLLWVPGSTGQVQLVESGGGVVQPGRSLRLSCAASGIDFSRYWMSWVRQAPGKGL
EWVGEINPDSSTINYAPSLKDRFTISRDNSKNTLYLQMSSLRAEDTAVYYCASLYYDYGDAMDYWG
QGTTVTVSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPCREEMTKNQVSLWCLVKGFYPSDI
AVEWESNGQPENNYKTTPPVLDSDGSFFLYSKLTVDKSRWQQGNVFSCSVMHEALHNHYTQKSLSL
SPGKGGGGSGGGGSGGGGSEVQLVESGGGLVQPGGSLRLSCAASGFTFSTYAMNWVRQAPGKGLE
WVSRIRSKYNNYATYYADSVKDRFTISRDDSKNTLYLQMNSLKTEDTAVYYCVRHGNFGNSYVSW
FAYWGQGTLVTVSSGGGGSGGGGSGGGGSGGGGSQAVVTQEPSLTVSPGGTVTLTCRSSTGAVTTS
NYANWVQQKPGQAPRGLIGGTNKRAPWTPARFSGSLLGGKAALTLSGAQPEDEAEYYCALWYSNL
WVFGGGTKLTVL
Molecule I.2
(light chain)
SEQ ID NO: 3277
METDTLLLWVLLLWVPGSTGDIQMTQSPSSLSASVGDRVTITCKASQSVDSNVAWYQQKPEKAPKA
LIFSASLRFSGVPSRFSGSGSGTDFTLTISSLQPEDFATYFCQQYNNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC
Molecule I.3
(heavy chain 2)
SEQ ID NO: 3287
METDTLLLWVLLLWVPGSTGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPI
EKTISKAKGQPREPQVCTLPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGK

In an aspect, a multispecific molecule as described herein comprises SEQ ID NO: 3286, SEQ ID NO: 3277, and/or SEQ ID NO: 3287 or a sequence with at least 85%, 90%, 95%, 96%, 97%, 98%, 99% or more identity thereto.

In some embodiments, the multispecific or multifunctional molecule as described herein binds to immune cells. In some embodiments, the multispecific or multifunctional molecule as described herein binds to subsets of immune cells. In some embodiments, the multispecific or multifunctional molecule as described herein binds to T cells. In some embodiments, the multispecific or multifunctional molecule as described herein binds to gamma/delta T cells. In some embodiments, the multispecific or multifunctional molecule as described herein binds to NKT cells. In some embodiments, the multispecific or multifunctional molecule as described herein binds to T cells via a binding moiety. Exemplary binding moieties include, but are not limited to, the binding moieties that bind to TRBC1, TRBC2, CD3, TRAC, TRAV subtypes. CD4. CD8, CD2, CD28, 41BB, PD1, CTLA4, OX40, TIM3, or LAG3. In some embodiments, the multispecific or multifunctional molecule as described herein binds to T cells via a binding moiety that binds to TRBC1. In some embodiments, the multispecific or multifunctional molecule as described herein binds to T cells via a binding moiety that binds to TRBC2. In some embodiments, the multispecific or multifunctional molecule as described herein comprises a binding moiety that binds to TRBC1. In some embodiments, the multispecific or multifunctional molecule as described herein comprises a binding moiety that binds to TRBC2.

In some embodiments, in exemplary embodiments of multifunctional molecules comprises a tumor-associated antigen (TAA)-binding moiety, a TCRβV-binding moiety, and at least one cytokine polypeptide as described herein, a tumor-associated antigen (TAA)-binding moiety and/or a TCRβV-binding moiety are antibody, an antigen binding fragment thereof or an antibody fragment. In some embodiments, in exemplary embodiments of multifunctional molecules comprises a tumor-associated antigen (TAA)-binding moiety, a TCRβV-binding moiety, and at least one cytokine polypeptide as described herein, an antigen binding fragment or an antibody fragment comprises Fab, Fab′. F(ab′)₂, F(ab)₂, variable fragment (Fv), domain antibody (dAb), or single chain variable fragment (scFv). In some embodiments, in exemplary embodiments of multifunctional molecules comprises a tumor-associated antigen (TAA)-binding moiety, a TCRβV-binding moiety, and at least one cytokine polypeptide as described herein, the at least one cytokine polypeptide is selected from the group consisting of interleukin-2 (IL-2) or a fragment or a functional fragment or a functional variant thereof, interleukin-7 (IL-7) or a fragment or a functional fragment or a functional variant thereof, interleukin-12 (IL-12) or a fragment or a functional fragment or a functional variant thereof, interleukin-15 (IL-15) or a fragment or a functional fragment or a functional variant thereof, interleukin-18 (IL-18) or a fragment or a functional fragment or a functional variant thereof, interleukin-21 (IL-21) or a fragment or a functional fragment or a functional variant thereof, or interferon gamma or a fragment or a functional fragment or a functional variant thereof, or a combination thereof. In some embodiments, exemplary embodiments of multifunctional molecules comprises a tumor-associated antigen (TAA)-binding moiety, a TCRβV-binding moiety, and at least one cytokine polypeptide as described herein comprises an exemplary dimerization module comprising an Fc region comprising N297A mutation. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region comprising Knob-in-bole mutations and disulfide bridges. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region comprising disulfide bridges, but not comprising Knob-in-hole mutations. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region not comprising disulfide bridges nor Knob-in-hole mutations. In some embodiments, in exemplary embodiment of multifunctional molecules comprising a tumor-associated antigen (TAA)-binding moiety, a TCRβV-binding moiety, and at least one cytokine polypeptide as described herein, the TCRβV-binding moiety comprises an scFv. In some embodiments, in exemplary embodiment of multifunctional molecules comprising a tumor-associated antigen (TAA)-binding moiety, a TCRβV-binding moiety, and at least one cytokine polypeptide as described herein, the TCRβV-binding moiety comprises an Fab.

In some embodiments, the multifunctional molecule as described herein comprises the at least one cytokine molecule or a functional fragment or functional variant thereof comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270.

In some embodiments, the multifunctional molecule as described herein comprises the at least one cytokine molecule or a functional fragment or functional variant thereof comprising the sequence of SEQ ID NO: 2270.

In some embodiments, the multifunctional molecule as described herein comprises the first Fc region, the second Fc region, or a combination thereof comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 40, SEQ ID NO: 42, SEQ ID NO: 3645, SEQ ID NO: 3646, SEQ ID NO: 3647. SEQ ID NO:3648, SEQ ID NO: 3649, SEQ ID NO: 3792, or SEQ ID NO: 3794.

In some embodiments, the multifunctional molecule as described herein comprises the first Fc region, the second Fc region, or a combination thereof comprising the sequence of SEQ ID NO. 40, SEQ ID NO: 42, SEQ ID NO: 3645, SEQ ID NO: 3646, SEQ ID NO: 3647, SEQ ID NO:3648, SEQ ID NO: 3649, SEQ ID NO: 3792, or SEQ ID NO: 3794.

In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649 or SEQ ID NO: 3792, the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, or a combination thereof. In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, or a combination thereof.

In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649 or SEQ ID NO: 3792, and the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794. In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, and the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794.

In some embodiments, the multifunctional molecule as described herein comprises the first Fc region comprising a sequence having the sequence of SEQ ID NO: 3649 or SEQ ID NO: 3792, the second Fc region comprising the sequence of SEQ ID NO: 3648 or SEQ ID NO: 3794, or a combination thereof. In some embodiments, the multifunctional molecule as described herein comprises the first Fc region comprising the sequence of SEQ ID NO: 3648 or SEQ ID NO: 3794, the second Fc region comprising the sequence of SEQ ID NO: 3648 or SEQ ID NO: 3794, or a combination thereof.

In some embodiments, the multifunctional molecule as described herein comprises the first Fc region comprising a sequence having the sequence of SEQ ID NO: 3649 or SEQ ID NO: 3792, and the second Fc region comprising the sequence of SEQ ID NO: 3648 or SEQ ID NO: 3794. In some embodiments, the multifunctional molecule as described herein comprises the first Fc region comprising the sequence of SEQ ID NO: 3648 or SEQ ID NO: 3794, and the second Fc region comprising the sequence of SEQ ID NO: 3648 or SEQ ID NO: 3794.

In some embodiments, the multifunctional molecule as described herein comprises the TCRβV-binding moiety comprising: (i) a VH comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to any one of the heavy chain variable domain amino acid sequences listed in Tables 1, 2, 10B, 11, 12, 13, 21, or 22; (ii) a VL comprising a sequence having at least at least 50%, 55%, 60%, 65%, 70%, 75%, 80%, 85%, 90%, 95%, 96%, 97%, 98%, 99%, 99.5%, 99.7%, 99.9% sequence identity to any one of the light chain variable domain amino acid sequences listed in Tables 1, 2, 10B, 11, 12, 13, 21, or 22; (iii) a combination thereof.

In some embodiments, the multifunctional molecule as described herein comprises the TCRβV-binding moiety comprising: (i) a VH comprising any one of the heavy chain variable domain amino acid sequences listed in Tables 1, 2, 10B, 11, 12, 13, 21, or 22; (ii) a VL comprising any one of the light chain variable domain amino acid sequences listed in Tables 1, 2, 10B, 11, 12, 13, 21, or 22; (iii) a combination thereof.

In some embodiments, the multifunctional molecule as described herein comprises the second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346, a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO. 1349, a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270, and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO. 3648.

In some embodiments, the multifunctional molecule as described herein comprises the second polypeptide chain comprising the sequence of SEQ ID NO: 1346, the sequence of SEQ ID NO: 1349, the sequence of SEQ ID NO: 2270, and the sequence of SEQ ID NO: 3648.

In some embodiments, the multifunctional molecule as described herein comprises the second polypeptide chain further comprising the sequence of SEQ ID NO: 3801, the sequence of SEQ ID NO: 3309, the sequence of SEQ ID NO: 3308, or any combination thereof.

In some embodiments, the multifunctional molecule as described herein comprises the second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 via the sequence of SEQ ID NO: 3801.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 via the sequence of SEQ ID NO: 3309.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to the sequence having at least 50%, 55%, 60%, 65%, 70%, 75%, 80%, 85%, 90%, 95%, 96%, 97%, 98%, 99%, 99.5%. 99.7%, or 99.9% sequence identity to the sequence of SEQ ID NO: 3648 via the sequence of SEQ ID NO: 3308.

In some embodiments, the multifunctional molecule as described herein comprises the second polypeptide chain comprising the sequence of SEQ ID NO: 1346 operatively linked to the sequence of SEQ ID NO: 1349 operatively linked to the sequence of SEQ ID NO: 2270 operatively linked to the sequence of SEQ ID NO: 3648.

In some embodiments, the multifunctional molecule as described herein comprises the sequence of SEQ ID NO: 1346 operatively linked to the sequence of SEQ ID NO: 1349 via the sequence of SEQ ID NO: 3801.

In some embodiments, the multifunctional molecule as described herein comprises the sequence of SEQ ID NO: 1349 operatively linked to the sequence of SEQ ID NO: 2270 via the sequence of SEQ ID NO: 3309.

In some embodiments, the multifunctional molecule as described herein comprises the sequence of SEQ ID NO: 2270 operatively linked to the sequence of SEQ ID NO: 3648 via the sequence of SEQ ID NO: 3308.

In some embodiments, the multifunctional molecule as described herein comprises the second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3800.

In some embodiments, the multifunctional molecule as described herein comprises the second polypeptide chain comprising the sequence of SEQ ID NO: 3800.

In some embodiments, the multifunctional molecule as described herein comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to any component sequences listed in Table 21, e.g., the VH sequences of TCRvβ listed in Table 21, the VL sequences of TCRvβ listed in Table 21, Fc region sequences listed in Table 21, cytokine sequences listed in Table 21, immunoglobulin light chain constant region sequences listed in Table 21, and linker sequences listed in Table 21, or any combination thereof. In some embodiments, the multifunctional molecule as described herein comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to any chain or polypeptide chain sequences listed in Table 21.

In some embodiments, the multifunctional molecule as described herein comprises any component sequences listed in Table 21, e.g., the VH sequences of TCRvβ listed in Table 21, the VL sequences of TCRvβ listed in Table 21. Fc region sequences listed in Table 21, cytokine sequences listed in Table 21, immunoglobulin light chain constant region sequences listed in Table 21, and linker sequences listed in Table 21, or any combination thereof. In some embodiments, the multifunctional molecule as described herein comprises any chain or polypeptide chain sequences listed in Table 21.

In some embodiments, the multifunctional molecule as provided herein comprises, from the N-terminus to the C-terminus, the following configuration:
A-, B-[dimerization module]-C, -D.

In some embodiments, the dimerization module comprises a first immunoglobulin chain constant region and a second immunoglobulin chain constant region.

In some embodiments. A and C are linked to the first immunoglobulin chain constant region; and B and D are linked to the second immunoglobulin chain constant region.

In some embodiments, A or C is the tumor-associated antigen binding moiety.

In some embodiments, B and D are independently absent or at least one cytokine molecule or a functional fragment or functional variant thereof.

In some embodiments, when A is the tumor-associated antigen binding moiety, C is absent or at least one cytokine molecule or a functional fragment or functional variant thereof.

In some embodiments, when C is the tumor-associated antigen binding moiety. A is absent or at least one cytokine molecule or a functional fragment or functional variant thereof.

In some embodiments, the Fc region comprises one or more mutations that modify the binding to the Fc gamma receptors. In some embodiments, the Fc region comprises one or more mutations that reduce the binding to Fc gamma receptors. In some embodiments, the Fc region comprises N297A mutation.

In some embodiments the multifunctional molecule consists of two polypeptide chains. In some embodiments the multifunctional molecule consists of two polypeptide chains, wherein the first polypeptide chain comprises an anti-tumor antigen binder, e.g., an scFv that binds to a tumor antigen. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain that comprises an anti-TCRvB binder linked to a cytokine, e.g., IL-2, wherein the cytokine is linked to an Fc, e.g., a human IgG1 knob cys N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain comprises, from N to C terminus an anti-TCRvB binder linked to a cytokine, e.g., IL-2, linked to an Fc. e.g., a human IgG1 knob cys N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain comprises SEQ ID NO 3800.

In some embodiments the multifunctional molecule consists of three polypeptide chains. In some embodiments the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab. In some embodiments the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 bole N297A. In some embodiments the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab; and the second polypeptide chain that comprises an anti-TCRvB binder linked to a cytokine, e.g., IL-2, wherein the cytokine is linked to an Fc, e.g., a human IgG1 knob cys N297A. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g, heavy chain variable domain of a Fab, linked to an Fc, e.g., a human IgG1 hole N297A; and the second polypeptide chain comprises, from N to C terminus an anti-TCRvB binder linked to a cytokine, e.g., IL-2, linked to an Fc, e.g., a human IgG1 knob cys N297A. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 hole N297A: and the second polypeptide chain comprises SEQ ID NO 3800. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g, heavy chain variable domain of a Fab, linked to an Fc, e.g., a human IgG1 hole N297A; and the second polypeptide chain comprises, from N to C terminus an anti-TCRvB binder linked to a cytokine, e.g., IL-2, linked to an Fc, e.g., a human IgG1 knob cys N297A; and the third polypeptide chain comprises a second portion of the anti-tumor antigen binder that binds to a tumor antigen, e.g., a second portion of the Fab, e.g., a light chain variable domain of the Fab. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g, heavy chain variable domain of a Fab, linked to an Fc, e.g., a human IgG1 hole N297A; and the second polypeptide chain comprises SEQ ID NO 3800; and the third polypeptide chain comprises a second portion of the anti-tumor antigen binder that binds to a tumor antigen, e.g., a second portion of the Fab, e.g., a light chain variable domain of the Fab.

### Linkers

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 3307): GGGGSGGGGS (SEQ ID NO: 3308); GGGGSGGGGSGGGGS (SEQ ID NO: 3309): DVPSGPGGGGGSGGGGS (SEQ ID NO: 3310); and GGGGGGGGSGGGGGS (SEQ ID NO: 3643). In some embodiments, the peptide linker is a A (EAAAK) nA (SEQ ID NO: 3437) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3314): AEAAAKEAAAKEAAAKAAA (SEQ ID NO: 3315): AEAAAKEAAAKEAAAKEAAAKAAA (SEQ ID NO: 3316); and AEAAAKEAAAKEAAAKEAAAKEAAAKAAA (SEQ ID NO: 3317).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRβV antibody molecules, multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or baving one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions) In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein) In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRβV antibody molecules, or a multispecific or multifunctional molecule described herein. In some embodiments, the vectors comprise nucleic acid sequences encoding antibody molecules, e.g., anti-TCRβV antibody molecules, or multispecific or multifunctional molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette.” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells. COS cells, CHO cells, HEK293 cells, BHK cells and MDCKII cells. Suitable insect cells include, but are not limited to, Sf9 cells.

Method of Expanding Cells with Anti-TCRVB Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells. B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In some embodiments, the methods of the application can utilize culture media conditions comprising DMEM. DMEM F12. RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 μM). IN some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8): 3975-3977, 1998; Haanen et al., J. Exp. Med. 190(9): 13191328, 1999; Garland et al., J. Immunol Meth. 227(1-2):53-63, 1999).

A TIL population can also be expanded by methods known in the art. For example, a population of TILs can be expanded as described in Hall et al., Journal for Immuno Therapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a T_{EMRA} cell or a TIL population) can be expanded by contacting the immune cell population with an anti-TCRVB antibody, e.g., as described herein.

In some embodiments, the expansion occurs in vivo, e.g., in a subject. In some embodiments, a subject is administered the multispecific or multifunctional molecules comprising TCRβV-binding moieties as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multispecific or multifunctional molecules as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion as described herein comprises culturing, e.g., expanding, the cells for at least about 4 hours, 6 hours, 10 hours, 12 hours, 15 hours. 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multispecific or multifunctional molecules as described herein can expand, e.g., selectively or preferentially expand, T cells expressing a T cell receptor (TCR) comprising a TCR alpha and/or TCR beta molecule, e.g., TCR alpha-beta T cells (αβ T cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 5, Table 6, and Table 7. Unless otherwise specified. CRS as used herein refers to CRS according to the criteria of Table 6.

In some embodiments, CRS is graded according to Table 5.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a functional fragment or a functional variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a functional fragment or a functional variant thereof. In some embodiments, a cytokine profile comprises the level and/or activity of a naturally occurring chemokine, a fragment or a functional fragment or a functional variant thereof. In some embodiments, a cytokine profile comprises the level and/or activity of one or more of: IL-2 (e.g., full length, a variant, or a fragment thereof); IL-1beta (e.g., full length, a variant, or a fragment thereof); IL-6 (e.g., full length, a variant, or a fragment thereof): TNFα (e.g., full length, a variant, or a fragment thereof); IFNgamma (e.g., full length, a variant, or a fragment thereof) IL-10 (e.g., full length, a variant, or a fragment thereof); IL-4 (e.g., full length, a variant, or a fragment thereof): TNF alpha (e.g., full length, a variant, or a fragment thereof): IL-12p70 (e.g., full length, a variant, or a fragment thereof); IL-13 (e.g., full length, a variant, or a fragment thereof): IL-8 (e.g., full length, a variant, or a fragment thereof): Eotaxin (e.g., full length, a variant, or a fragment thereof): Eotaxin-3 (e.g., full length, a variant, or a fragment thereof): IL-8 (HA) (e.g., full length, a variant, or a fragment thereof): IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-1a (e.g., full length, a variant, or a fragment thereof): MIP-1b (e.g., full length, a variant, or a fragment thereof): TARC (e.g., full length, a variant, or a fragment thereof): GM-CSF (e.g., full length, a variant, or a fragment thereof): IL-12 23p40 (e.g., full length, a variant, or a fragment thereof): IL-15 (e.g., full length, a variant, or a fragment thereof); IL-1β (e.g., full length, a variant, or a fragment thereof): IL-17a (e.g., full length, a variant, or a fragment thereof): IL-1a (e.g., full length, a variant, or a fragment thereof): IL-5 (e.g., full length, a variant, or a fragment thereof): IL-7 (e.g., full length, a variant, or a fragment thereof): TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRβV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergie response, etc., and are commensurate with a reasonable benefit/risk ratio. (Sec, e.g., S. M. Berge, et al., J. Pharmaceutical Sciences, 66:1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quatemary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427. “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment.” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes. (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease.” or “disorder.” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Cancer Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma. Non-Hodgkin's lymphoma, acute myeloid leukemia (AML), chronic myeloid leukemia, myelodysplastic syndrome, multiple myeloma. T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma. Burkitt lymphoma, lymphoplasmacytic lymphoma, hairy cell leukemia, and a combination thereof. In some embodiments, the T-cell lymphoma is peripheral T-cell lymphoma.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, DLL3. CLDN18.2, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2. Melan-A/MART-1, Gp100/pmel17, Tyrosinase, TRP-1/-2, MC1R, β-catenin, BRCA 1/2, CDK4, CML66, Fibronectin, p53, Ras, TGF-B receptor, AFP, ETA, MAGE, MUC-1, CA-125, BAGE, GAGE, NY-ESO-1, β-catenin. CDK4, CDC27, α actinin-4, TRP 1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Integrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRβV molecules in a subject. In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Without wishing to be bound by theory, it is believed that a biased T cell population, e.g., a T cell population expressing a TCRβV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRβV molecules associated with the cancer. In some embodiments, the TCRβV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRβV molecules in a sample from the subject, comprising contacting said immune effector cell population with an anti-TCRβV antibody molecule as described herein, e.g., an anti-TCRβV antibody molecule that binds to the same TCRβV molecule that is higher, e.g., increased in the immune effector cell population in the sample from the subject. In some embodiments, contacting the population of immune effector cells (e.g., comprising T cells that express one or more TCRβV molecules) with an anti-TCRβV molecule results in expansion of the population of immune effector cells expressing one or more TCRβV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRβV molecules in a sample from the subject, and determining whether the one or more TCRβV molecules is higher, e.g., increased, in a sample from the subject compared to a reference value, wherein responsive to said determination, administering to the subject an effective amount of an anti-TCRβV antibody molecule, e.g., an agonistic anti-TCRβV antibody molecule, e.g., as described herein.

In some embodiments, the subject has B-CLL. In some embodiments, a subject having B-CLL has a higher, e.g., increased, level or activity of one or more TCRβV molecules, e.g., one or more TCRβV molecules comprising: (i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01; (ii) TCRβ V5 subfamily comprising TCRβ V5-6*01, TCRβ V5-4*01, or TCRβ V5-8*01; (iii) TCRβ V3 subfamily comprising TCRβ V3-1*01; (iv) TCRβ V2 subfamily comprising TCRβ V2*01; or (v) TCRβ V19 subfamily comprising TCRβ V19*01, or TCRβ V19*02.

In some embodiments, a subject having B-CLL has a higher, e.g., increased, level or activity of a TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, a subject having B-CLL has a higher, e.g., increased, level or activity of a TCRβ V5 subfamily comprising TCRβ V5-6*01, TCRβ V5-4*01, or TCRβ V5-8*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V5 subfamily. In some embodiments, administration of the multifunctional polypeptide molecule as described herein results in expansion of immune cells expressing one or more members of the TCRβ V5 subfamily.

In some embodiments, a subject having B-CLL has a higher, e.g., increased, level or activity of a TCRβ V3 subfamily comprising TCRβ V3-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V3 subfamily. In some embodiments, administration of the multifunctional polypeptide molecule as described herein results in expansion of immune cells expressing one or more members of the TCRβ V3 subfamily.

In some embodiments, a subject having B-CLL has a higher, e.g., increased, level or activity of a TCRβ V2 subfamily comprising TCRβ V2*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V2 subfamily. In some embodiments, administration of the multifunctional polypeptide molecule as described herein results in expansion of immune cells expressing one or more members of the TCRβ V2 subfamily.

In some embodiments, a subject having B-CLL has a higher, e.g., increased, level or activity of a TCRβ V19 subfamily comprising TCRβ V19*01, or TCRβ V19*02. In some embodiments, the subject is administered the multifunctional polypeptide molecule as described herein comprising an anti-TCRβV molecule (e.g., an agonistic anti-TCRβV molecule as described herein) that binds to one or more members of the TCRβ V19 subfamily. In some embodiments, administration of the multifunctional polypeptide molecule as described herein results in expansion of immune cells expressing one or more members of the TCRβ V19 subfamily.

In some embodiments, the subject has melanoma. In some embodiments, a subject having melanoma has a higher, e.g., increased, level or activity of one or more TCRβV molecules, e.g., one or more TCRβV molecules comprising the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has DLBCL (diffuse large B-cell lymphoma). In some embodiments, a subject having DLBCL has a higher, e.g., increased, level or activity of one or more TCRβV molecules, e.g., one or more TCRβV molecules comprising: (i) TCRβ V13 subfamily comprising TCRβ V13*01; (ii) TCRβ V3 subfamily comprising TCRβ V3-1*01; or (iii) TCRβ V23 subfamily.

In some embodiments, a subject having DLBCL has a higher, e.g., increased, level or activity of a TCRβ V13 subfamily comprising TCRβ V13*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V13 subfamily. In some embodiments, administration of the multifunctional polypeptide molecule as described herein results in expansion of immune cells expressing one or more members of the TCRβ V13 subfamily.

In some embodiments, a subject having DLBCL has a higher, e.g., increased, level or activity of a TCRβ V3 subfamily comprising TCRβ V3-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V3 subfamily. In some embodiments, administration of the multifunctional polypeptide molecule as described herein results in expansion of immune cells expressing one or more members of the TCRβ V3 subfamily.

In some embodiments, a subject having DLBCL has a higher, e.g., increased, level or activity of a TCRβ V23 subfamily. In some embodiments, the subject is administered the multifunctional polypeptide molecule as described herein comprising an anti-TCRβV molecule (e.g., an agonistic anti-TCRβV molecule as described herein) that binds to one or more members of the TCRβ V23 subfamily. In some embodiments, administration of the multifunctional polypeptide molecule as described herein results in expansion of immune cells expressing one or more members of the TCRβ V23 subfamily.

In some embodiments, the subject has CRC (colorectal cancer). In some embodiments, a subject having CRC has a higher, e.g., increased, level or activity of one or more TCRβV molecules, e.g., one or more TCRβV molecules comprising: (i) TCRβ V19 subfamily comprising TCRβ V19*01, or TCRβ V19*02; (ii) TCRβ V12 subfamily comprising TCRβ V12-4*01, TCRβ V12-3*01, or TCRβ V12-5*01; (iii) TCRβ V16 subfamily comprising TCRβ V16*01; or (iv) TCRβ V21 subfamily.

In some embodiments, a subject having CRC has a higher, e.g., increased, level or activity of a TCRβ V19 subfamily comprising TCRβ V19*01, or TCRβ V19*02. In some embodiments, the subject is administered the multifunctional polypeptide molecule as described herein comprising an anti-TCRβV molecule (e.g., an agonistic anti-TCRβV molecule as described herein) that binds to one or more members of the TCRβ V19 subfamily. In some embodiments, administration of the multifunctional polypeptide molecule as described herein results in expansion of immune cells expressing one or more members of the TCRβ V19 subfamily.

In some embodiments, a subject having CRC has a higher, e.g., increased, level or activity of a TCRβ V12 subfamily comprising TCRβ V12-4*01, TCRβ V12-3*01, or TCRβ V12-5*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V12 subfamily. In some embodiments, administration of the multifunctional polypeptide molecule as described herein results in expansion of immune cells expressing one or more members of the TCRβ V12 subfamily.

In some embodiments, a subject having CRC has a higher, e.g., increased, level or activity of a TCRβ V16 subfamily comprising TCRβ V16*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V16 subfamily. In some embodiments, administration of the multifunctional polypeptide molecule as described herein results in expansion of immune cells expressing one or more members of the TCRβ V16 subfamily.

In some embodiments, a subject having CRC has a higher, e.g., increased, level or activity of a TCRβ V21 subfamily. In some embodiments, the subject is administered the multifunctional polypeptide molecule as described herein comprising an anti-TCRβV molecule (e.g., an agonistic anti-TCRβV molecule as described herein) that binds to one or more members of the TCRβ V21 subfamily. In some embodiments, administration of the multifunctional polypeptide molecule as described herein results in expansion of immune cells expressing one or more members of the TCRβ V21 subfamily.

In some embodiments, acquiring a value for the status, e.g., presence, level and/or activity, of one or more TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRβV molecules is obtained, e.g., measured, using an assay described in Wang C Y, et al., Int J Oncol (2016) 48 (6): 2247-56, the entire contents of which are hereby incorporated by reference.

In some embodiments, a value for the status of one or more TCRβV molecules is obtained, e.g., measured, using flow cytometry.

### Combination Therapies

In some embodiments, the method as described herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the nucleic acid molecules as described herein, the vector as described herein, the cell as described herein, the pharmaceutical composition as described herein, sequentially, simultaneously, or concurrently.

The multispecific or multifunctional molecules as described herein can be used in combination with a second therapeutic agent or procedure.

In some embodiments, the multispecific or multifunctional molecules as described herein and the second therapeutic agent or procedure are administered/performed after a subject has been diagnosed with a cancer, e.g., before the cancer has been eliminated from the subject. In some embodiments, the multispecific or multifunctional molecules as described herein and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecules as described herein and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or cancer cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In some embodiments, the anti-TCRβV antibody, multispecific or multifunctional molecule is administered in combination with a therapy, e.g., a cancer therapy (e.g, one or more of anti-cancer agents, immunotherapy, photodynamic therapy (PDT), surgery and/or radiation). The terms “chemotherapeutic.” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the anti-TCRβV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P, et al. (2011) CA Cancer J. Clin. 61.250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g. B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia. AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency. Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma. Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma. Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the multispecific or multifunctional molecules as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g. Rosenberg et al., New Eng. J, of Med. 319:1676, 1988).

In some embodiments, the multispecific or multifunctional molecules as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J, of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the multispecific or multifunctional molecules as described herein is administered in combination with a low or small molecular weight chemotherapeutic agent Exemplary low or small molecular weight chemotherapeutic agents include, but not limited to, 13-cis-retinoic acid (isotretinoin. ACCUTANE®), 2-CdA (2-chlorodeoxyadenosine, cladribine, LEUSTATIN™), 5-azacitidine (azacitidine. VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomyein, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BICNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXANR, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard. MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecules as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab. Genentech Inc., South San Francisco, Calif.; a humanized monoclonal antibody that has anti-tumor activity in HER2-positive breast cancer); FASLODEX® (fulvestrant, AstraZeneca Pharmaceuticals. LP, Wilmington. Del.; an estrogen-receptor antagonist used to treat breast cancer): ARIMIDEX® (anastrozole, AstraZeneca Pharmaceuticals, LP: a nonsteroidal aromatase inhibitor which blocks aromatase, an enzyme needed to make estrogen): Aromasin® (exemestane, Pfizer Inc., New York, N.Y.; an irreversible, steroidal aromatase inactivator used in the treatment of breast cancer); FEMARA® (lctrozole. Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules as described herein may be combined include: AVASTIN® (bevacizumab. Genentech Inc.; the first FDA-approved therapy designed to inhibit angiogenesis); and ZEVALIN® (ibritumomab tiuxetan, Biogen Idec. Cambridge, Mass.: a radiolabeled monoclonal antibody currently approved for the treatment of B-cell lymphomas).

In addition, the FDA has approved the following biologics for the treatment of colorectal cancer; AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate: a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.: a small molecule designed to target the human epidermal growth factor receptor 1 (HER1) pathway).

For the treatment of multiple myeloma, exemplary biologics include VELCADE® (bortezomib, Millennium Pharmaceuticals, Cambridge Mass.: a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan. ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®), RITUXAN®), robatumumab, satumomab pendetide, sibrotuzumab, siltuximab, sontuzumab, tacatuzumab tetraxetan (AFP-CIDER), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In some embodiments, the multispecific or multifunctional molecules as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001. Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine. MVA-EBNA1/LMP2 Inj, vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine: recombinant vaccinia-CEA (6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN. HspE7. FP253/Fludarabine, ALVAC (2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the multispecific or multifunctional molecules as described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecules as described herein are administered in combination with paclitaxel or a paclitaxel formulation, e.g., TAXOL R, protein-bound paclitaxel (e.g. ABRAXANE®). Exemplary paclitaxel formulations include, but are not limited to, nanoparticle albumin-bound paclitaxel (ABRAXANE®, marketed by Abravis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug Elute®), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b). ROFERON-A® (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®). Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), Jeuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g. BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate. ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®. SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the multispecific or multifunctional molecules as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®) CGP57148B, STI-571), lapatinib (TYKERBR_TYVERBR), lestaurtinib (CEP-701), neratinib (HKI-272), nilotinib (TASIGNA®), semavanib (scmaxinib, SU5416), sunitinib (SUTENT®, SUI 1248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647. XL228, AEE788. AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the multispecific or multifunctional molecules as described herein are administered in combination with one of more of an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab). VEGF receptor inhibitors (e.g., itraconazole), inhibitors of cell proliferatin and/or migration of endothelial cells (e.g. carboxy amidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe. P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecules as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4 (2012): 252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04. ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475. SCH-900475 or KEYTRUDA®; Merck) is a humanized IgG4 monoclonal antibody that binds to PD-1. See, e.g. Hamid, O, et al. (2013) New England Journal of Medicine 369 (2): 134-44. U.S. Pat. No. 8,354,509 and WO2009/114335 Pidilizumab (also called CT-011 or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US2010028330, and/or US20120114649.

In some embodiments, the PD-1 inhibitor is an immunoadhesin, e.g., an immunoadhesin comprising an extracellular/PD-1 binding portion of a PD-1 ligand (e.g. PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In some embodiments, the PD-L1 inhibitor is an anti-PD-L1 antibody, e.g., YW243.55.S70. The YW243.55.S70 antibody is described, e.g., in WO 2010/077634. In some embodiments, the PD-L1 inhibitor is MDX-1105 (also called BMS-936559), which is described, e.g., in WO2007/005874. In some embodiments, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In some embodiments, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010. CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoictic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells. B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes. B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM. DMEM F12. RPMI 1640, and/or AIM V media. The media can be supplemented with glutamine. HEPES buffer (e.g., 10 mM), scrum (e.g., heat-inactivated serum, e.g., 10%), and/or beta mercaptoethanol (e.g., 55 μM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190 (9):13191328, 1999; Garland et al., J. Immunol Meth 227(1-2):53-63, 1999).

In some embodiments, a TIL population can also be expanded by methods known in the art. For example, a population of TILs can be expanded as described in Hall et al., Journal for Immuno Therapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours. 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g. selectively or preferentially expand, T cells expressing a T cell receptor (TCR) comprising a TCR alpha and/or TCR beta molecule, e.g., TCR alpha-beta T cells (αβ T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands αβ T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRβV region results in one, two, three or all of: (1) reduced T cell proliferation kinetics; (ii) cell killing, e.g., target cell killing, e.g. cancer cell killing, e.g., as measured by an assay of Example 4; (iii) increased Natural Killer (NK) cell proliferation, e.g., expansion; or (iv) expansion, e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRβV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule or the multispecific or multifunctional molecules as described herein.

In some embodiments, expansion of the population of memory effector T cells, e.g., TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of. T cells expressing a T cell receptor (TCR) comprising a TCR alpha and/or TCR beta molecule, e.g., TCR alpha-beta T cells (αβ T cells).

In some embodiments, the method results in expansion of αβT cells over expansion of T cells expressing a TCR comprising a TCR gamma and/or TCR delta molecule, e.g., TCR gamma-delta T cells (γδ T cells). In some embodiments, expansion of αβT cells over γδ T cells results in reduced production of cytokines associated with CRS. In some embodiments, expansion of αβT cells over γδ T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor beta variable (TCRβV) region. In some embodiments, binding of the multifunctional polypeptide molecule as described herein results in expansion. e.g., at least about 1.1-50 fold expansion (e.g., at least about 1.5-40 fold, 2-35 fold, 3-30 fold, 5-25 fold, 8-20 fold, or 10-15 fold expansion), of a population of T cells, e.g., a population of T cells having a memory-like phenotype, e.g., CD45RA+ CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g. T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+ CCR7− CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity, e.g., as compared to a reference cell population, e.g., an otherwise similar population of cells that has not been contacted with an anti-TCRβV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype, e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule, e.g., CD3 epsilon (CD3e) molecule; or a TCR alpha (TCRa) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype. T effector memory cells, comprises CD3+ and CD8+ T cells. In some embodiments, the population of expanded T cells having a memory-like phenotype, e.g., T effector memory cells comprises CD3+ and CD4+ T cells.

In some embodiments, the population of expanded T cells having a memory-like phenotype, T effector memory (TEM) cells, comprises cells T cells, e.g., CD3+, CD8+ or CD4+ T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein, e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein, e.g., flow cytometry.

In some embodiments, the population of T cells having a memory-like phenotype, e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+, CCR7− and CD95+ cells. In some embodiments, the population of T cells having a memory-like phenotype, e.g., TEMRA cells comprise CD3+, CD4+ or CD8+ T cells (e.g., CD3+ T cells, CD3+ CD8+ T cells, or CD3+ CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRGI. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein, e.g., a method described in Examples 10 and 35. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion). In some embodiments, the expansion is compared to expansion of a similar population of cells with an antibody that binds to a CD3 molecule, e.g., CD3 epsilon (CD3e) molecule; or a TCR alpha (TCRα) molecule.

In some embodiments, binding of the multifunctional polypeptide molecule as described herein results in proliferation, e.g., expansion, e.g., at least about 1.1-50 fold expansion (e.g., at least about 1.5-40 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing, e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional polypeptide molecule as described herein to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding, e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional polypeptide molecule as described herein comprising an antibody molecule, e.g., humanized antibody molecule, which binds, e.g., specifically binds, to a T cell receptor beta variable chain (TCRβV) region (e.g., anti-TCRβV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject: e.g., a peripheral blood sample, biopsy sample, or bone marrow sample; (ii) acquiring a value of the status of one or more TCRβV molecules for the subject, e.g., in the biological sample from the subject, wherein said value comprises a measure of the presence of, e.g., level or activity of, a TCRβV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Accordingly, provided herein are, inter alia, multispecific or multifunctional molecules comprising TCRβV-binding moieties as described herein (e.g., multispecific or multifunctional antibody molecules) that comprise anti-TCRβV antibody molecules, nucleic acids encoding the same, methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer, e.g., as described herein.

### Exemplary Multifunctional Polypeptide Molecule

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 3523, the sequence of SEQ ID NO: 2170, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%. 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO. 3649; (ii) a second polypeptide chain comprising, from the N-terminus to the C-terminus, the sequence of SEQ ID NO: 3523 operatively linked to the sequence of SEQ ID NO: 2170 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3524, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3523 is operatively linked to the sequence of SEQ ID NO: 2170 via the sequence of SEQ ID NO: 3524, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3519; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO. 3517; (ii) a second polypeptide chain comprising the sequence of SEQ ID NO: 3519; and (iii) a third polypeptide chain comprising to the sequence of SEQ ID NO: 3518.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%. 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO. 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising the sequence of SEQ ID NO. 1346, and the sequence of SEQ ID NO: 3649; (ii) a second polypeptide chain comprising the sequence of SEQ ID NO: 2170, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%. 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO. 3649; (ii) a second polypeptide chain comprising, from the N-terminus to the C-terminus, the sequence of SEQ ID NO: 2170 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3520; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3520; and (iii) a third polypeptide chain comprising to the sequence of SEQ ID NO: 3518.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide chain comprising the sequence of SEQ ID NO. 1346, and the sequence of SEQ ID NO: 3649; (ii) a second polypeptide chain comprising the sequence of SEQ ID NO: 2270, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%. 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO. 3649; (ii) a second polypeptide chain comprising, from the N-terminus to the C-terminus, the sequence of SEQ ID NO: 2270 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2270 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3521; and (iii) a third polypeptide chain comprising to the sequence of SEQ ID NO: 3518.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530, and a sequence having at least 50%, 55%, 60%, 65%, 70%, 75%, 80%, 85%, 90%, 95%, 97%, 98%, 99%. 99.5%, 99.6%, 99.7%, 99.8%, or 99.9% sequence identity to the sequence of SEQ ID NO: 3531; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide chain comprising the sequence of SEQ ID NO: 3530, and the sequence of SEQ ID NO: 3531; (ii) a second polypeptide chain comprising the sequence of SEQ ID NO: 2191, and the sequence of SEQ ID NO: 3533; and (iii) a third polypeptide chain comprising the sequence of SEQ ID NO: 3527, and the sequence of SEQ ID NO: 3528.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide chain comprising, from the N-terminus to the C-terminus, the sequence of SEQ ID NO: 3530 operatively linked to the sequence of SEQ ID NO: 3531; (ii) a second polypeptide chain comprising, from the N-terminus to the C-terminus, the sequence of SEQ ID NO: 2191 operatively linked to the sequence of SEQ ID NO: 3533; and (iii) a third polypeptide chain comprising, from the N-terminus to the C-terminus, the sequence of SEQ ID NO: 3527 operatively linked to the sequence of SEQ ID NO: 3528.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2191 is operatively linked to the sequence of SEQ ID NO: 3533 via the sequence of SEQ ID NO: 3308, or a combination thereof.

In some embodiments, the first polypeptide chain further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3547 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531, the second polypeptide chain further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3534 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533, or a combination thereof.

In some embodiments, the first polypeptide chain further comprises the sequence of SEQ ID NO: 3547 operatively linked to the sequence of SEQ ID NO: 3531, the second polypeptide chain further comprises the sequence of SEQ ID NO: 3534 operatively linked to the sequence of SEQ ID NO: 3533, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3529 or the sequence of SEQ ID NO: 3548; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3532 or the sequence of SEQ ID NO: 3549; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3526.

In some embodiments, the multifunctional polypeptide molecule comprises: (i) a first polypeptide chain comprising the sequence of SEQ ID NO. 3529 or the sequence of SEQ ID NO: 3548; (ii) a second polypeptide chain comprising the sequence of SEQ ID NO: 3532 or the sequence of SEQ ID NO: 3549; and (iii) a third polypeptide chain comprising to the sequence of SEQ ID NO: 3526.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%. 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 3540, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 3540 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3540 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%. 99.7%, 99.8%, or 99.9% sequence identity to the sequence of SEQ ID NO: 3539; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3539; and (iii) a third polypeptide chain comprising to the sequence of SEQ ID NO: 3518.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3542, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648, and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO. 3542, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3542 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 3542 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the IL-12 molecule or a functional fragment or a functional variant thereof comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 and a sequence having at least 50%, 55%, 60%, 65%, 70%, 75%, 80%, 85%, 90%, 95%, 97%, 98%, 99%, 99.5%. 99.6%, 99.7%, 99.8%, or 99.9% sequence identity to the sequence of SEQ ID NO: 3545.

In some embodiments, the IL-12 molecule or a functional fragment or a functional variant thereof comprises the sequence of SEQ ID NO: 3543 and the sequence of SEQ ID NO: 3545.

In some embodiments, the IL-12 molecule or a functional fragment or a functional variant thereof comprises, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO:3545.

In some embodiments, the IL-12 molecule or a functional fragment or a functional variant thereof comprises, from the N-terminus to the C-terminus, the sequence of SEQ ID NO: 3543 operatively linked to the sequence of SEQ ID NO:3545.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3545 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3544, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3545 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3543 is operatively linked to the sequence of SEQ ID NO: 3545 via the sequence of SEQ ID NO: 3544, the sequence of SEQ ID NO: 3545 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3541; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3541; and (iii) a third polypeptide chain comprising to the sequence of SEQ ID NO: 3518.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 2193, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2193 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO. 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 2193 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2193 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308.

In some embodiments, the sequence of SEQ ID NO: 2193 is operatively linked to the sequence of SEQ ID NO: 3648 via the sequence of SEQ ID NO: 3308.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3546; and (iii) a third polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3546; and (iii) a third polypeptide chain comprising to the sequence of SEQ ID NO: 3518.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises: (i) a first polypeptide chain comprising the sequence of SEQ ID NO. 3530 and the sequence of SEQ ID NO: 3537; and (ii) a second polypeptide chain comprising the sequence of SEQ ID NO: 3527, the sequence of SEQ ID NO: 3528, and the sequence of SEQ ID NO: 2191.

In some embodiments, the multifunctional polypeptide molecule comprises: (i) a first polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide chain comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises: (i) a first polypeptide chain comprising, from the N-terminus to the C-terminus, the sequence of SEQ ID NO: 3530 operatively linked to the sequence of SEQ ID NO: 3537; and (ii) a second polypeptide chain comprising, from the N-terminus to the C-terminus, the sequence of SEQ ID NO: 3527 operatively linked to the sequence of SEQ ID NO: 3528 operatively linked to the sequence of SEQ ID NO: 2191.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3309.

In some embodiments, the sequence of SEQ ID NO: 3528 is operatively linked to the sequence of SEQ ID NO: 2191 via the sequence of SEQ ID NO: 3309.

In some embodiments, the multifunctional polypeptide molecule comprises two first polypeptide chains and two second polypeptide chains.

In some embodiments, the multifunctional polypeptide molecule comprises: (i) a first polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3536; and (ii) a second polypeptide chain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3535.

In some embodiments, the multifunctional polypeptide molecule comprises: (i) a first polypeptide chain comprising the sequence of SEQ ID NO: 3536; and (ii) a second polypeptide chain comprising the sequence of SEQ ID NO: 3535.

In some embodiments, the multifunctional polypeptide molecule comprises: (i) a first polypeptide chain comprising one or more components as listed in Table 21; and (ii) a second polypeptide chain comprising one or more components as listed in Table 21. In some embodiments, the multifunctional polypeptide molecule further comprises: (i) a third polypeptide chain comprising one or more components as listed in Table 21; and (ii) a fourth polypeptide chain comprising one or more components as listed in Table 21.

In some embodiments, the multifunctional polypeptide molecule comprises: (i) a first polypeptide chain comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21; and (ii) a second polypeptide chain comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21. In some embodiments, the multifunctional polypeptide molecule further comprises: (i) a third polypeptide chain comprising one or more components comprising a sequence having at least 50%, 55%, 60%, 65%, 70%, 75%, 80%, 85%, 90%, 95%, 97%, 98%, 99%, 99.5%, 99.6%. 99.7%, 99.8%, or 99.9% sequence identity to any one of the component sequences as listed in Table 21; and (ii) a fourth polypeptide chain comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21.

In some embodiments, the multifunctional polypeptide molecule comprises: (i) a first polypeptide chain comprising one or more component sequences as listed in Table 21; and (ii) a second polypeptide chain comprising one or more component sequences as listed in Table 21. In some embodiments, the multifunctional polypeptide molecule further comprises: (i) a third polypeptide chain comprising one or more component sequences as listed in Table 21; and (ii) a fourth polypeptide chain comprising one or more component sequences as listed in Table 21.

In some embodiments, the multifunctional polypeptide molecule comprises: (i) a first polypeptide chain comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21; and (ii) a second polypeptide chain comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21. In some embodiments, the multifunctional polypeptide molecule further comprises: (i) a third polypeptide chain comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21; and (ii) a fourth polypeptide chain comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21.

In some embodiments, the multifunctional polypeptide molecule comprises: (i) a first polypeptide chain comprising any one of the polypeptide sequences as listed in Table 21; and (ii) a second polypeptide chain comprising any one of the polypeptide sequences as listed in Table 21. In some embodiments, the multifunctional polypeptide molecule further comprises: (i) a third polypeptide chain comprising any one of the polypeptide sequences as listed in Table 21; and (ii) a fourth polypeptide chain comprising one or more comprising any one of the polypeptide sequences as listed in Table 21.

In another aspect, described herein is an antibody comprising an anti-T cell receptor beta variable chain (TCRβV) binding domain comprising: (i) a heavy chain variable region (VH) comprising a heavy chain complementarity determining region 1 (HC CDR1), a heavy chain complementarity determining region 2 (HC CDR2), and a heavy chain complementarity determining region 3 (HC CDR3) comprising an amino acid sequence having at least 50%, 55%, 60%, 65%, 70%, 75%, 80%, 85%, 90%, 95%, 97%, 98%, 99%, 99.5%. 99.6%, 99.7%, 99.8%, or 99.9% sequence identity to SEQ ID NO: 3650, SEQ ID NO: 3651, and SEQ ID NO: 5, respectively; (ii) a light chain variable region (VL) comprising a light chain complementarity determining region 1 (LC CDR1), a light chain complementarity determining region 2 (LC CDR2), and a light chain complementarity determining region 3 (LC CDR3) comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 3655, SEQ ID NO: 3653, and SEQ ID NO: 8, respectively; or (iii) a combination thereof.

In some embodiments, the TCRβV binding domain comprising: (i) a VH comprising a HC CDR1, a HC CDR2, and a HC CDR3 comprising the amino acid sequence of SEQ ID NO: 3650, SEQ ID NO: 3651, and SEQ ID NO: 5, respectively; (ii) a VL comprising a LC CDR1, a LC CDR2, and a LC CDR3 comprising the amino acid sequence of SEQ ID NO: 3655, SEQ ID NO: 3653, and SEQ ID NO: 8, respectively; or (iii) a combination thereof.

In some embodiments, the TCRβV binding domain comprising: (i) a VH comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1346; (ii) a VL comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1349; or (iii) a combination thereof.

In some embodiments, the TCRβV binding domain comprising: (i) a VH comprising the amino acid sequence of SEQ ID NO: 1346; (ii) a VL comprising the amino acid sequence of SEQ ID NO: 1349; or (iii) a combination thereof.

In some embodiments, the antibody comprising an anti-T cell receptor beta variable chain (TCRβV) binding domain further comprises a cytokine polypeptide or a functional fragment or a functional variant thereof. In some embodiments, the cytokine polypeptide may be IL-2, IL2-C125A, IL-15, IL-7, IL-12, or IL-21. In embodiments, the cytokine polypeptide may further comprise a cytokine receptor. In some embodiments, the cytokine polypeptide may comprise IL-15 linked to a IL-15Ra. In some embodiments, the cytokine polypeptide may comprise IL-15 linked to a IL-15Ra sushi domain. In some embodiments, the cytokine polypeptide may comprise a cytokine dimer. In some embodiments, the cytokine polypeptide may comprise an IL-12 beta subunit linked to an IL-12 alpha subunit.

TABLE 1
Amino acid and nucleotide sequences for murine, chimeric and humanized antibody molecules
which bind to TCRVB 6, e.g., TCRVB 6-5. The antibody molecules include murine m.Ab Antibody A,
and humanized mAb Antibody A-H Clones A-H.1 to A-H.85. The amino acid the heavy and light
chain CDRs, and the amino acid and nucleotide sequences of the heavy and light chain variable
regions, and the heavy and light chains are shown.
Antibody A (murine), also referred to as H131, TCRVB 6-5 binder

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQLS |
| | | SLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (Chothia) | SSSHRYS |
| SEQ ID NO: 56 | LC CDR3 (Chothia) | QQFKSYPLT |
| SEQ ID NO: 2 | VL | DILMTQSQKFMSTSLGDRVSVSCKASQNVGINVVWHQQKPG |
| | | QSPKALIYSSSHRYSGVPDRFTGSGSGTDFTLTINNVQSEDLA |
| | | EYFCQQFKSYPLTFGAGTKLELK |

Antibody A humanized (A-H antibody), TCRVB 6-5 binder
A-H.1 antibody (also referred to as BHM1709)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: 69 | VH-IgM constant delta | METDTLLLWVLLLWVPGSTGQVQLVQSGAEVKKPGSSVKVS |
| | CDC | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSGSASAPTLFPLVSCENSPSDTSSVAVG |
| | | CLAQDFLPDSITFSWKYKNNSDISSTRGFPSVLRGGKYAATSQ |
| | | VLLPSKDVMQGTDEHVVCKVQHPNGNKEKNVPLPVIAELPP |
| | | KVSVFVPPRDGFFGNPRKSKLICQATGFSPRQIQVSWLREGKQ |
| | | VGSGVTTDQVQAEAKESGPTTYKVTSTLTIKESDWLGQSMFT |
| | | CRVDHRGLTFQQNASSMCVPDQDTAIRVFAIPPSFASIFLTKS |
| | | TKLTCLVTDLTTYDSVTISWTRQNGEAVKTHTNISESHPNATE |
| | | SAVGEASICEDDWNSGERFTCTVTHTDLASSLKQTISRPKGVA |
| | | LHRPDVYLLPPAREQLNLRESATITCLVTGFSPADVFVQWMQ |
| | | RGQPLSPEKYVTSAPMPEPQAPGRYFAHSILTVSEEEWNTGET |
| | | YTCVVAHEALPNRVTERTVDKSTGKPTLYNVSLVMSDTAGT |
| | | CY |
| SEQ ID NO: 70 | VH-IgGA1 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLCSTQPDGNVVIACL |
| | | VQGFFPQEPLSVTWSESGQGVTARNFPPSQDASGDLYTTSSQ |
| | | LTLPATQCLAGKSVTCHVKHYTNPSQDVTVPCPVPSTPPTPSP |
| | | STPPTPSPSCCHPRLSLHRPALEDLLLGSEANLTCILTGLRDAS |
| | | GVTFTWTPSSGKSAVQGPPERDLCGCYSVSSVLPGCAEPWNH |
| | | GKTFTCTAAYPESKTPLTATLSKSGNTFRPEVHLLPPPSEELAL |
| | | NELVTLTCLARGFSPKDVLVRWLQGSQELPREKYLTWASRQ |
| | | EPSQGTTTFAVTSILRVAAEDWKKGDTFSCMVGHEALPLAFT |
| | | QKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLDSTPQDGNVVVACL |
| | | VQGFFPQEPLSVTWSESGQNVTARNFPPSQDASGDLYTTSSQ |
| | | LTLPATQCPDGKSVTCHVKHYTNSSQDVTVPCRVPPPPPCCH |
| | | PRLSLHRPALEDLLLGSEANLTCTLTGLRDASGATFTWTPSSG |
| | | KSAVQGPPERDLCGCYSVSSVLPGCAQPWNHGETFTCTAAH |
| | | PELKTPLTANITKSGNTFRPEVHLLPPPSEELALNELVTLTCLA |
| | | RGFSPKDVLVRWLQGSQELPREKYLTWASRQEPSQGTTTYA |
| | | VTSILRVAAEDWKKGETFSCMVGHEALPLAFTQKTIDRMAG |
| | | KPTHINVSVVMAEADGTCY |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKQFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPGK |
| | | APKALIYSSSHRYSGVPSRFSGSGSGTEFTLTISSLQPEDFATYF |
| | | CQQFKSYPLTFGQGTKLEIK |
| SEQ ID NO: 13 | DNA VL | GACATCCAGATGACCCAGTCTCCATCCTTCCTGTCCGCCTC |
| | | TGTGGGCGACAGAGTGACCATCACATGCAAGGCCTCTCAG |
| | | AACGTGGGCATCAACGTCGTGTGGCACCAGCAGAAGCCTG |
| | | GCAAGGCTCCTAAGGCTCTGATCTACTCCTCCAGCCACCG |
| | | GTACTCTGGCGTGCCCTCTAGATTTTCCGGCTCTGGCTCTG |
| | | GCACCGAGTTTACCCTGACAATCTCCAGCCTGCAGCCTGA |
| | | GGACTTCGCCACCTACTTTTGCCAGCAGTTCAAGAGCTACC |
| | | CTCTGACCTTTGGCCAGGGCACCAAGCTGGAAATCAAG |
| SEQ ID NO: 72 | VL and kappa constant | METDTLLLWVLLLWVPGSTGDIQMTQSPSFLSASVGDRVTIT |
| | region/light chain | CKASQNVGINVVWHQQKPGKAPKALIYSSSHRYSGVPSRFSG |
| | | SGSGTEFTLTISSLQPEDFATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.2 antibody (also referred to as BHM1710)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TOKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPGK |
| | | VPKALIYSSSHRYSGVPSRFSGSGSGTDFTLTISSLOPEDVATY |
| | | FCQQFKSYPLTFGQGTKLEIK |
| SEQ ID NO: 14 | DNA VL | GACATCCAGATGACCCAGTCTCCATCCTCTCTGTCCGCCTC |
| | | TGTGGGCGACAGAGTGACCATCACATGCAAGGCCTCTCAG |
| | | AACGTGGGCATCAACGTCGTGTGGCACCAGCAGAAACCTG |
| | | GCAAGGTGCCCAAGGCTCTGATCTACTCCTCCAGCCACAG |
| | | ATACTCCGGCGTGCCCTCTAGATTCTCCGGCTCTGGCTCTG |
| | | GCACCGACTTTACCCTGACAATCTCCAGCCTGCAGCCTGA |
| | | GGACGTGGCCACCTACTTTTGCCAGCAGTTCAAGAGCTAC |
| | | CCTCTGACCTTTGGCCAGGGCACCAAGCTGGAAATCAAG |
| SEQ ID NO: | Light chain | METDTLLLWVLLLWVPGSTGDIQMTQSPSSLSASVGDRVTIT |
| 3279 | | CKASQNVGINVVWHQQKPGKVPKALIYSSSHRYSGVPSRFSG |
| | | SGSGTDFTLTISSLQPEDVATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 81 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 82 | VH | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDEDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 84 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 85 | VH | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.5

| | | |
|---|---|---|
| SEQ ID NO: 86 | VH + VL | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 87 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 88 | VH | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 90 | VL | DIQMTQSPSFLSASVGDRVTITCKASQNVDNRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 91 | VH | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVENKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: 93 | VL | DIQMTQSPSFLSASVGDRVTITCKASQNVENKVAWHQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 94 | VH | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 96 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 97 | VH | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQAP |
| | | GQQLEWMGRVSAGSQNVKYNEKFKGRVTITADTSTSTAYME |
| | | LSSLRSEDTAVYYCAGSYYSYDVLDYWGQGTTVTVSSGGGG |
| | | SGGGGSGQGGSGGGGSDIQMTQSPSFLSASVGDRVTITCKAS |
| | | QNVGNRVAWYQQKPGKAPKALIYSSSHRYSGVPSRFSGSGS |
| | | GTEFTLTISSLQPEDFATYFCQQFKSYPLTFGQGTKLEIK |
| SEQ ID NO: 99 | VL | DIQMTQSPSFLSASVGDRVTITCKASQNVGNRVAWYQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 100 | | GQGLEWMGRVSAGSGNVKYNEKFKGRVTITADTSTSTAYME |
| | | LSSLRSEDTAVYYCAGSYYSYDVLDYWGQGTTVTVSS |

A-H.10

| | | |
|---|---|---|
| SEQ ID NO: | VH + VL | QVQLVQSGAEVKKPGSSVKVSCKASGHDEDKFYIHWVRQAP |
| 101 | | GQGLEWMGRIFAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIKS |
| SEQ ID NO: | VL | DIQMTQSPSFLSASVGDRVTITCKASQNVGDRVAWYQQKPG |
| 102 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 103 | | GQGLEWMGRIFAGSGNVKYNEKFKGRVTITADTSTSTAYME |
| | | LSSLRSEDTAVYYCAGSYYSYDVLDYWGQGTTVTVSS |

A-H.11

| | | |
|---|---|---|
| SEQ ID NO: | VH + VL | QVQLVQSGAEVKKPGSSVKVSCKASGTDFKLTYIHWVRQAP |
| 104 | | GQGLEWMGRVSPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 105 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 106 | | GQGLEWMGRVSPGSGNVKYNEKFKGRVTITADTSTSTAYME |
| | | LSSLRSEDTAVYYCAGSYYSYDVLDYWGQGTTVTVSS |

A-H.12

| | | |
|---|---|---|
| SEQ ID NO: | VH + VL | QVQLVQSGAEVKKPGSSVKVSCKASGTDFDKIYIHWVRQAP |
| 107 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 108 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 109 | | GQGLEWMGRVSAGSGNTKYNEKFKGRVTITADTSTSTAYME |
| | | LSSLRSEDTAVYYCAGSYYSYDVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 111 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 112 | | GQGLEWMGRIFPGSGNVKYNEKFKGRVTITADTSTSTAYME |
| | | LSSLRSEDTAVYYCAGSYYSYDVLDYWGQGTTVTVSS |

A-H.14

| | | |
|---|---|---|
| SEQ ID NO: | VH + VL | QVQLVQSGAEVKKPGSSVKVSCKASGTDEDKIYIHWVRQAP |
| 113 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 114 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 115 | | GQGLEWMGRISAGSGNTKYNEKFKGRVTITADTSTSTAYME |
| | | LSSLRSEDTAVYYCAGSYYSYDVLDYWGQGTTVTVSS |

A-H.15

| | | |
|---|---|---|
| SEQ ID NO: | VH + VL | QVQLVQSGAEVKKPGSSVKVSCKASGTDFRLTYIHWVRQAP |
| 116 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPQ |
| 117 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 118 | | GQGLEWMGRVSPGSGNTKYNEKFKGRVTITADTSTSTAYME |
| | | LSSLRSEDTAVYYCAGSYYSYDVLDYWGQGTTVTVSS |

A-H.16

| | | |
|---|---|---|
| SEQ ID NO: | VH + VL | QVQLVQSGAEVKKPGSSVKVSCKASGGTFRLTYIHWVRQAP |
| 119 | | GQGLEWMGRVYPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 120 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 121 | | GQGLEWMGRVYPGSGNTKYNEKFKGRVTITADTSTSTAYME |
| | | LSSLRSEDTAVYYCAGSYYSYDVLDYWGQGTTVTVSS |

A-H.17

| | | |
|---|---|---|
| SEQ ID NO: | VH + VL | QVQLVQSGAEVKKPGSSVKVSCKASGTDFRLTYIHWVRQAP |
| 122 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 123 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 124 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.18

| | | |
|---|---|---|
| SEQ ID NO: | VH + VL | QVQLVQSGAEVKKPGSSVKVSCKASGTDFKLTYIHWVRQAP |
| 125 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 126 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 127 | | GQGLEWMGRIFPGSGNVKYNEKFKGRVTITADTSTSTAYME |
| | | LSSLRSEDTAVYYCAGSYYSYDVLDYWGQGTTVTVSS |

A-H.19

| | | |
|---|---|---|
| SEQ ID NO: | VH + VL | QVQLVQSGAEVKKPGSSVKVSCKASGGTFRLTYIHWVRQAP |
| 128 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 129 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 130 | | GQGLEWMGRISAGSQNVKYNEKFKGRVTITADTSTSTAYME |
| | | LSSLRSEDTAVYYCAGSYYSYDVLDYWGQGTTVTVSS |

A-H.20

| | | |
|---|---|---|
| SEQ ID NO: | VH + VL | QVQLVQSGAEVKKPGSSVKVSCKASGQTFDKTYIHWVRQAP |
| 131 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 132 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFDKTYIHWVRQAP |
| 133 | | GQGLEWMGRISAGSGNTKYNEKFKGRVTITADTSTSTAYME |
| | | LSSLRSEDTAVYYCAGSYYSYDVLDYWGQGTTVTVSS |

A-H.21

| | | |
|---|---|---|
| SEQ ID NO: | VH + VL | QVQLVQSGAEVKKPGSSVKVSCKASGHDFDKFYIHWVRQAP |
| 134 | | CQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGQSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 135 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 136 | | GQGLEWMGRISAGSQNTKYNEKFKGRVTITADTSTSTAYME |
| | | LSSLRSEDTAVYYCAGSYYSYDVLDYWGQGTTVTVSS |

A-H.22

| | | |
|---|---|---|
| SEQ ID NO: | VH + VL | QVQLVQSGAEVKKPGSSVKVSCKASGTDFKLTYIHWVRQAP |
| 137 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 138 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 139 | | GQGLEWMGRISAGSGNVKYNEKFKGRVTITADTSTSTAYME |
| | | LSSLRSEDTAVYYCAGSYYSYDVLDYWGQGTTVTVSS |

A-H.23

| | | |
|---|---|---|
| SEQ ID NO: | VH + VL | QVQLVQSGAEVKKPGSSVKVSCKASGHDFRLTYIHWVRQAP |
| 140 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 141 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFRLTYIHWVRQAP |
| 142 | | GQGLEWMGRISAGSGNVKYNEKFKGRVTITADTSTSTAYME |
| | | LSSLRSEDTAVYYCAGSYYSYDVLDYWGQGTTVTVSS |

A-H.24

| | | |
|---|---|---|
| SEQ ID NO: | VH + VL | QVQLVQSGAEVKKPGSSVKVSCKASGHDFHLWYIHWVRQA |
| 143 | | PGQGLEWMGRVSAGSGNVKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDNKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 144 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 145 | | PGQGLEWMGRVSAGSGNVKYNEKFKGRVTITADTSTSTAYM |
| | | ELSSLRSEDTAVYYCAGSYYSYDVLDYWGQGTTVTVSS |

A-H.25

| | | |
|---|---|---|
| SEQ ID NO: | VH + VL | QVQLVQSGAEVKKPGSSVKVSCKASGHDFHLWYIHWVRQA |
| 146 | | PGQGLEWMGRVFAGSGNTKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDKVAWYQQKPGKAPKALIYSSSHRYKQVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDKVAWYQQKPG |
| 147 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 148 | | PGQGLEWMGRVFAGSGNTKYNEKFKGRVTITADTSTSTAYM |
| | | ELSSLRSEDTAVYYCAGSYYSYDVLDYWGQGTTVTVSS |

A-H.26

| | | |
|---|---|---|
| SEQ ID NO: | VH + VL | QVQLVQSGAEVKKPGSSVKVSCKASGTDFKLTYIHWVRQAP |
| 149 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 150 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 151 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.27

| | | |
|---|---|---|
| SEQ ID NO: | VH + VL | QVQLVQSGAEVKKPGSSVKVSCKASGTDFKLTYIHWVRQAP |
| 153 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 154 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 155 | | GQGLEWMGRVSAGSGNVKYNEKFKGRVTITADTSTSTAYME |
| | | LSSLRSEDTAVYYCAGSYYSYDVLDYWGQGTTVTVSS |

A-H.28

| | | |
|---|---|---|
| SEQ ID NO: | VH + VL | QVQLVQSGAEVKKPGSSVKVSCKASGTDFKLTYIHWVRQAP |
| 156 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 157 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 158 | | GQGLEWMGRISPGSGNTKYNEKFKGRVTITADTSTSTAYMEL |
| | | SSLRSEDTAVYYCAGSYYSYDVLDYWGQGTTVTVSS |

A-H.29

| | | |
|---|---|---|
| SEQ ID NO: | VH + VL | QVQLVQSGAEVKKPGSSVKVSCKASGHDFHLWYIHWVRQA |
| 159 | | PGQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWHQQKPG |
| 160 | | KAPKALIYSSSHRYKGVPSRFSGSGSQTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 161 | | PGQGLEWMGRISPGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGHDFKLTYIHWVRQAP |
| 162 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 163 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 164 | | GQGLEWMGRISAGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGTDFHLWYIHWVRQAP |
| 165 | | GQGLEWMGRVFAGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 166 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFHLWYIHWVRQAP |
| 167 | | GQGLEWMGRVFAGSGSYRYNEKFKGRVTITADTSTSTAYME |
| | | LSSLRSEDTAVYYCAGSYYSYDVLDYWGQGTTVTVSS |

A-H.32

| | | |
|---|---|---|
| SEQ ID NO: | VH + VL | QVQLVQSGAEVKKPGSSVKVSCKASGTDFDKIYIHWVRQAP |
| 168 | | GQQGLEWMGRISAGSQNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 169 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 170 | | GQGLEWMGRISAGSGNTKYNEKFKGRVTITADTSTSTAYME |
| | | LSSLRSEDTAVYYCAGSYYSYDVLDYWGQGTTVTVSS |

A-H.33

| | | |
|---|---|---|
| SEQ ID NO: | VH + VL | QVQLVQSGAEVKKPGSSVKVSCKASGTDFKLTYIHWVRQAP |
| 171 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 172 | | KAPKALIYSSSHRYKGVPSRFSGSGSQTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 173 | | GQGLEWMGRISAGSGNTKYNEKFKGRVTITADTSTSTAYME |
| | | LSSLRSEDTAVYYCAGSYYSYDVLDYWGQGTTVTVSS |

A-H.34

| | | |
|---|---|---|
| SEQ ID NO: | VH + VL | QVQLVQSGAEVKKPGSSVKVSCKASGTDFRLTYIHWVRQAP |
| 174 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGNRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 175 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 176 | | GQGLEWMGRISPGSGNTKYNEKFKGRVTITADTSTSTAYMEL |
| | | SSLRSEDTAVYYCAGSYYSYDVLDYWGQGTTVTVSS |

A-H.35

| | | |
|---|---|---|
| SEQ ID NO: | VH + VL | QVQLVQSGAEVKKPGSSVKVSCKASGHDEDKTYIHWVRQAP |
| 177 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 178 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 179 | | GQGLEWMGRVSAGSGNVKYNEKFKGRVTITADTSTSTAYME |
| | | LSSLRSEDTAVYYCAGSYYSYDVLDYWGQGTTVTVSS |

A-H.36

| | | |
|---|---|---|
| SEQ ID NO: | VH + VL | QVQLVQSGAEVKKPGSSVKVSCKASGHDFKLTYIHWVRQAP |
| 180 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWHQQKPGKAPKALIYSSSHRYKGVPSRFSQSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWHQQKPG |
| 181 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 182 | | GQGLEWMGRVSPGSGNTKYNEKFKGRVTITADTSTSTAYME |
| | | LSSLRSEDTAVYYCAGSYYSYDVLDYWGQGTTVTVSS |

A-H.37

| | | |
|---|---|---|
| SEQ ID NO: | VH + VL | QVQLVQSGAEVKKPGSSVKVSCKASGHDEDKTYIHWVRQAP |
| 183 | | GQGLEWMGRIYPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VI | DIQMTQSPSFLSASVGDRVTITCKASQNVADRVAWYQQKPG |
| 184 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDEDKTYIHWVRQAP |
| 185 | | GQGLEWMGRIYPGSGNVKYNEKFKGRVTITADTSTSTAYME |
| | | LSSLRSEDTAVYYCAGSYYSYDVLDYWGQGTTVTVSS |

A-H.38

| | | |
|---|---|---|
| SEQ ID NO: | VH + VL | QVQLVQSGAEVKKPGSSVKVSCKASGTDFDKTYIHWVRQAP |
| 186 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 187 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKTYIHWVRQAP |
| 188 | | GQGLEWMGRISAGSGNVKYNEKFKGRVTITADTSTSTAYME |
| | | LSSLRSEDTAVYYCAGSYYSYDVLDYWGQGTTVTVSS |

A-H.39

| | | |
|---|---|---|
| SEQ ID NO: | VH + VL | QVQLVQSGAEVKKPGSSVKVSCKASGTDFDKIYIHWVRQAP |
| 189 | | GQGLEWMGRISAGSGNIKYNEKFKGRVTITADTSTSTAYMEL |
| | | SSLRSEDTAVYYCAGSYYSYDVLDYWGQGTTVTVSSGGGGS |
| | | GGGGSGGGGGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 190 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 191 | | GQGLEWMGRISAGSGNIKYNEKFKGRVTITADTSTSTAYMEL |
| | | SSLRSEDTAVYYCAGSYYSYDVLDYWGQGTTVTVSS |

A-H.40

| | | |
|---|---|---|
| SEQ ID NO: | VH + VL | QVQLVQSGAEVKKPGSSVKVSCKASGTDFDKIYIHWVRQAP |
| 192 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 193 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 194 | | GQGLEWMGRISAGSGNVKYNEKFKGRVTITADTSTSTAYME |
| | | LSSLRSEDTAVYYCAGSYYSYDVLDYWGQGTTVTVSS |

A-H.41

| | | |
|---|---|---|
| SEQ ID NO: | VH + VL | QVQLVQSGAEVKKPGSSVKVSCKASGGTFKLTYIHWVRQAP |
| 195 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 196 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFKLTYIHWVRQAP |
| 197 | | GQGLEWMGRVSAGSQNVKYNEKFKGRVTITADTSTSTAYME |
| | | LSSLRSEDTAVYYCAGSYYSYDVLDYWGQGTTVTVSS |

A-H.42

| | | |
|---|---|---|
| SEQ ID NO: | VH + VL | QVQLVQSGAEVKKPGSSVKVSCKASGTDFKLTYIHWVRQAP |
| 198 | | GQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWHQQKPG |
| 199 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 200 | | GQGLEWMGRISPGSGNVKYNEKFKGRVTITADTSTSTAYME |
| | | LSSLRSEDTAVYYCAGSYYSYDVLDYWGQGTTVTVSS |

A-H.43

| | | |
|---|---|---|
| SEQ ID NO: | VH + VL | QVQLVQSGAEVKKPGSSVKVSCKASGHDFDKFYIHWVRQAP |
| 201 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 202 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 203 | | GQGLEWMGRVSAGSGNTKYNEKFKGRVTITADTSTSTAYME |
| | | LSSLRSEDTAVYYCAGSYYSYDVLDYWGQGTTVTVSS |

A-H.44

| | | |
|---|---|---|
| SEQ ID NO: | VH + VL | QVQLVQSGAEVKKPGSSVKVSCKASGTDEDKFYIHWVRQAP |
| 204 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVVWYQQKPGKAPKALIYSSSHRYKGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGTDEDKFYIHWVRQAP |
| 205 | | GQGLEWMGRVSAGSGNVKYNEKFKGRVTITADTSTSTAYME |
| | | LSSLRSEDTAVYYCAGSYYSYDVLDYWGQGTTVTVSS |

A-H.45

| | | |
|---|---|---|
| SEQ ID NO: | VH + VL | QVQLVQSGAEVKKPGSSVKVSCKASGYSFTTYYIHWVRQAP |
| 206 | | GQGLEWMGWFSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 207 | | GQGLEWMGWFSAGSGNTKYNEKFKGRVTITADTSTSTAYME |
| | | LSSLRSEDTAVYYCAVSYYSYDVLDYWGQGTTVTVSS |

A-H.46

| | | |
|---|---|---|
| SEQ ID NO: | VH + VL | QVQLVQSGAEVKKPGSSVKVSCKASGYSFTTYYIHWVRQAP |
| 208 | | GQGLEWMGWFSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 209 | | GQGLEWMGWFSAGSGNTKYNEKFKGRVTITADTSTSTAYME |
| | | LSSLRSEDTAVYYCAGSYYSYDVLDYWGQGTTVTVSS |

A-H.47

| | | |
|---|---|---|
| SEQ ID NO: | VH + VL | QVQLVQSGAEVKKPGSSVKVSCKASGYSFTTYYIHWVRQAP |
| 210 | | GQGLEWMGWFFPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 211 | | GQGLEWMGWFFPGSGNTKYNEKFKGRVTITADTSTSTAYME |
| | | LSSLRSEDTAVYYCAGSYYSYDVLDYWGQGTTVTVSS |

A-H.48

| | | |
|---|---|---|
| SEQ ID NO: | VH + VL | QVQLVQSGAEVKKPGSSVKVSCKASGYSFTTYYIHWVRQAP |
| 212 | | GQGLEWMGWFSPGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 213 | | GQGLEWMGWFSPGSGNTKYNEKFKGRVTITADTSTSTAYME |
| | | LSSLRSEDTAVYYCAVSYYSYDVLDYWGQGTTVTVSS |

A-H.49

| | | |
|---|---|---|
| SEQ ID NO: | VH + VL | QVQLVQSGAEVKKPGSSVKVSCKASGYSFTTYYIHWVRQAP |
| 214 | | GQGLEWMGWFSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 215 | | GQGLEWMGWFSPGSGNTKYNEKFKGRVTITADTSTSTAYME |
| | | LSSLRSEDTAVYYCAGSYYSYDVLDYWGQGTTVTVSS |

A-H.50

| | | |
|---|---|---|
| SEQ ID NO: | VH + VL | QVQLVQSGAEVKKPGSSVKVSCKASGYSFTTYYIHWVRQAP |
| 216 | | GQGLEWMGRIFPGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 217 | | GQGLEWMGRIFPGSGNIKYNEKFKGRVTITADTSTSTAYMEL |
| | | SSLRSEDTAVYYCAGSYYSYDVLDYWGQGTTVTVSS |

A-H.51

| | | |
|---|---|---|
| SEQ ID NO: | VH + VL | QVQLVQSGAEVKKPGSSVKVSCKASGYSFTTYYIHWVRQAP |
| 218 | | GQGLEWMGWFFPGSGNIKYNEKFKGRVTITADTSTSTAYME |
| | | LSSLRSEDTAVYYCAGSIYSAGVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 219 | | GQGLEWMGWFFPGSGNIKYNEKFKGRVTITADTSTSTAYME |
| | | LSSLRSEDTAVYYCAGSIYSAGVLDYWGQGTTVTVSS |

A-H.52

| | | |
|---|---|---|
| SEQ ID NO: | VH + VL | QVQLVQSGAEVKKPGSSVKVSCKASGYSFTLGYIHWVRQAP |
| 220 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTLGYIHWVRQAP |
| 221 | | GQGLEWMGWFFPGSGNIKYNEKFKGRVTITADTSTSTAYME |
| | | LSSLRSEDTAVYYCAGSYYSYDVLDYWGQGTTVTVSS |

A-H.53

| | | |
|---|---|---|
| SEQ ID NO: | VH + VL | QVQLVQSGAEVKKPGSSVKVSCKASGYSFRLTYIHWVRQAP |
| 222 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFRLTYIHWVRQAP |
| 223 | | GQGLEWMGWFFPGSGNIKYNEKFKGRVTITADTSTSTAYME |
| | | LSSLRSEDTAVYYCAGSYYSYDVLDYWGQGTTVTVSS |

A-H.54

| | | |
|---|---|---|
| SEQ ID NO: | VH + VL | QVQLVQSGAEVKKPGSSVKVSCKASGYSFHNWYIHWVRQA |
| 224 | | PGQGLEWMGWFFPGSGNIKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFHNWYIHWVRQA |
| 225 | | PGQGLEWMGWFFPGSGNIKYNEKFKGRVTITADTSTSTAYM |
| | | ELSSLRSEDTAVYYCAGSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIHWVRQAP |
| 1100 | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWHQQLPGKA |
| 1101 | | PKALIYSSSHRYSGVSDRFSGSGSGTSFSLAISGLQSEDEADYF |
| | | CQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDFDKFYIHWVRQAP |
| 1309 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |

A-H.57

| | | |
|---|---|---|
| SEQ ID NO: | VH + VL (ScFv) | QVQLVQSGABVKKPGSSVKVSCKASGHDFRLTYIHWVRQAP |
| 1326 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.58

| | | |
|---|---|---|
| SEQ ID NO: | VH + VL (ScFv) | QVQLVQSGAEVKKPGSSVKVSCKASGHDFRLTYIHWVRQAP |
| 1327 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGNRVVWHQQKPGKAPKALIYSSSHRYKGVPSRFSGSGS |
| | | GTEFTLTISSLQPEDFATYFCQQFKSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTYIHWVRQAP |
| 1328 | | GQGLEWMGRIYAGSGNVKYNEKFKGRVTITADTSTSTAYME |
| | | LSSLRSEDTAVYYCAVSYYSYDVLDYWGQGTTVTVSSGGGG |
| | | SGGGGSGGGGGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.60

| | | |
|---|---|---|
| SEQ ID NO: | VH + VL (ScFv) | QVQLVQSGAEVKKPGSSVKVSCKASGHDFKLTYIHWVRQAP |
| 1329 | | GQGLEWMGRVSAGSQNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |

A-H.61

| | | |
|---|---|---|
| SEQ ID NO: | VH + VL (ScFv) | QVQLVQSGAEVKKPGSSVKVSCKASGHDFKLTYIHWVRQAP |
| 1330 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTYIHWVRQAP |
| 1331 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTYIHWVRQAP |
| 1332 | | GQGLEWMGRVYAGSGNTKYNEKFKGRVTITADTSTSTAYM |
| | | ELSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVEDRVVWHQQKPGKAPKALIYSSSHRYKGVPSRFSGSGS |
| | | GTEFTLTISSLQPEDFATYFCQQFKSYPLTFGQGTKLEIK |

A-H.64

| | | |
|---|---|---|
| SEQ ID NO: | VH + VL (ScFv) | QVQLVQSGAEVKKPGSSVKVSCKASGHDFKLTYIHWVRQAP |
| 1333 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.65

| | | |
|---|---|---|
| SEQ ID NO: | VH + VL (ScFv) | QVQLVQSGAEVKKPGSSVKVSCKASGHDFKLTYIHWVRQAP |
| 1334 | | GQGLEWMGRISAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.66

| | | |
|---|---|---|
| SEQ ID NO: | VH + VL (ScFv) | QVQLVQSGAEVKKPGSSVKVSCKASGHDFKLTYIHWVRQAP |
| 1335 | | GQGLEWMGRIYAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTYIHWVRQAP |
| 1336 | | GQGLEWMGRIFPGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H.68

| | | |
|---|---|---|
| SEQ ID NO: | VH + VL (ScFv) | QVQLVQSGAEVKKPGSSVKVSCKASGHDFRLTYIHWVRQAP |
| 1337 | | GQGLEWMGRISAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H humanized-matured VH

| | | |
|---|---|---|
| SEQ ID NO: | VH-humanized matured | QVQLVQSGAEVKKPGSSVKVSCKASGTDFKLTYIHWVRQAP |
| 1310 | 1 | GQGLEWMGRIFPGSGNVKYNEKFKGRVTITADTSTSTAYME |
| | | LSSLRSEDTAVYYCAGSYYSYDVLDYWGQGTTVTVSS |
| SEQ ID NO: | VH-humanized matured | QVQLVQSGAEVKKPGSSVKVSCKASGTDFKLTYIHWVRQAP |
| 1311 | 2 | GQGLEWMGRIFPGSGNVKYNEKFKGRVTITADTSTSTAYME |
| | | LSSLRSEDTAVYYCAVSYYSYDVLDYWGQGTTVTVSS |
| SEQ ID NO: | VH-humanized matured | QVQLVQSGAEVKKPGSSVKVSCKASGHDFRLTYIHWVRQAP |
| 1312 | 3 | GQGLEWMGRISAGSGNVKYNEKFKGRVTITADTSTSTAYME |
| | | LSSLRSEDTAVYYCAVSYYSYDVLDYWGQGTTVTVSS |

A-H humanized-matured VL

| | | |
|---|---|---|
| SEQ ID NO: | VL-humanized matured | DIQMTQSPSFLSASVGDRVTITCKASQNVDNRVAWYQQKPG |
| 1313 | 1 | KAPKALIYSSSHRYKGVPSRFSGSGSGTEFTLTISSLQPEDFAT |
| | | YFCQQFKSYPLTFGQGTKLEIK |
| SEQ ID NO: | VL-humanized matured | DIQMTQSPSFLSASVGDRVTITCKASQNVADRVAWYQQKPG |
| 1314 | 2 | KAPKALIYSSSHRYKGVPSRFSGSGSGTEFTLTISSLQPEDFAT |
| | | YFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 3652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGNRVVWHQQKPG |
| 1347 | (CDRs underlined) | KAPKALIYSSSHRYKGVPSRFSGSGSGTEFTLTISSLQPEDFAT |
| | | YFCQQFKSYPLTFGQGTKLEIK |

A-H.71

| | | |
|---|---|---|
| SEQ ID NO: | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1348 | (CDRs underlined) | GQGLEWMGRIYAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSQNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVAWHQQKPG |
| 1351 | (CDRs underlined) | KAPKALIYSSSHRYKGVPSRFSGSGSGTEFTLTISSLQPEDFAT |
| | | YFCQQFKSYPLTFGQGTKLEIK |

A-H.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSQNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPEDFAT |
| | | YFCQQFKSYPLTFGQGTKLEIK |

A-H.74

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKQVPSRFSGSGSGTEFTLTISSLQPEDFAT |
| | | YFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1356 | (CDRs underlined) | GQGLEWMGRVYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVEDRVVWHQQKPG |
| 1357 | (CDRs underlined) | KAPKALIYSSSHRYKGVPSRFSGSGSGTEFTLTISSLQPEDFAT |
| | | YFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSQNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDEKLTYIHWVRQAP |
| 1360 | (CDRs underlined) | GQGLEWMGRISAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTKYNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1362 | (CDRs underlined) | GQGLEWMGRIYAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKQ |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDEKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSQTEFTLTISSLQPEDFAT |
| | | YFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSQNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH (CDRs underlined) | QVQLVQSGAEVKKPGSSVKVSCKASGHDFRLTYIHWVRQAP |
| 1344 | | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

TABLE 2
Amino acid and nucleotide sequences for murine and humanized antibody molecules which
bind to TCRVB 12, e.g., TCRVB 12-3 or TCRVB 12-4. The antibody molecules include murine
mAb Antibody B and humanized mAb Antibody B-H.1 to B-H.6. The amino acid the heavy and
light chain CDRs, and the amino acid and nucleotide sequences of the heavy and light
chain variable regions, and the heavy and light
chains are shown.
Antibody B (murine), also referred to as 16G8

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFGMHWVRQ |
| | | APDKGLEWVAYISSGSSTIYYADTLKGRFTISRDNPKNTLFL |
| | | QMTSLRSEDTAMYYCARRGEGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYWYQQKSD |
| | | ASPKLWIYYTSNLAPGVPTRFSGSGSQNSYSLTISSMEGEDA |
| | | ATYYCQQFTSSPFTFGSGTKLEIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: | VH | EVQLVESGGQLVQPGGSLRLSCAASGFTFSNFGMHWVRQA |
| 3438 | | PGKGLEWVSYISSGSSTIYYADTLKGRFTISRDNAKNSLYLQ |
| | | MNSLRAEDTAVYYCARRGEGAMDYWGQGTTVTVSS |
| SEQ ID NO: 31 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCTCTCGGGACAACGCCAAGAACTCCC |
| | | TGTACCTGCAGATGAACAGCCTGAGAGCCGAGGACACC |
| | | GCCGTGTACTACTGTGCTAGAAGAGGCGAGGGCGCCATG |
| | | GATTATTGGGGCCAGGGAACCACAGTGACCGTGTCTAGC |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | EVQLVESGGGLVQPGGSLRLSCAASGFTFSNFGMHWVRQA |
| | | PGKGLEWVSYISSGSSTIYYADTLKGRFTISRDNSKNTLYLQ |
| | | MNSLRAEDTAVYYCARRGEGAMDYWGQGTTVTVSS |
| SEQ ID NO: 32 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTGCTAGAAGAGGCQAGGGCGCCATGG |
| | | ATTATTGGGGCCAGGGAACCACAGTGACCGTGTCTAGC |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | QVQLVESGGGVVQPGRSLRLSCAASGFTFSNFGMHWVRQ |
| | | APGKGLEWVAYISSGSSTIYYADTLKGRFTISRDNSKNTLY |
| | | LQMNSLRAEDTAVYYCARRGEGAMDYWGQGTTVTVSS |
| SEQ ID NO: 33 | DNA VH | CAGGTGCAGCTGGTGGAATCTGGTGGCGGAGTTGTGCAG |
| | | CCTGGCAGATCCCTGAGACTGTCTTGTGCCGCCTCTGGCT |
| | | TCACCTTCTCCAACTTCGGCATGCACTGGGTCCGACAGG |
| | | CCCCTGGAAAAGGATTGGAGTGGGTCGCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTQCTAGAAGAGGCGAGGGCGCCATGG |
| | | ATTATTGGGGCCAGGGAACCACAGTGACCGTGTCTAGC |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 26 | VL | DNQLTQSPSFLSASVGDRVTITCRASSSVNYIYWYQQKPGK |
| | | APKLLIYYTSNLAPGVPSRFSGSGSGNEYTLTISSLQPEDFAT |
| | | YYCQQFTSSPFTFGQGTKLEIK |
| SEQ ID NO: 34 | DNA VL | GATAACCAGCTGACCCAGTCTCCTAGCTTCCTGTCTGCCT |
| | | CTGTGGGCGACAGAGTGACAATTACCTGCCGGGCCTCCT |
| | | CCTCCGTGAACTACATCTACTGGTATCAGCAGAAGCCCG |
| | | GCAAGGCCCCTAAGCTGCTGATCTACTACACCTCCAATC |
| | | TGGCCCCTGGCGTGCCCTCTAGATTTTCCGGATCTGGCTC |
| | | CGGCAACGAGTATACCCTGACAATCTCCAGCCTGCAGCC |
| | | TGAGGACTTCGCCACCTACTACTGCCAGCAGTTCACCTC |
| | | CTCTCCATTCACCTTTGGCCAGGGCACCAAGCTGGAAAT |
| | | CAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 27 | VL | DNQLTQSPSSLSASVGDRVTITCRASSSVNYIYWYQQKPGK |
| | | APKLLIYYTSNLAPGVPSRFSGSGSGNDYTLTISSLQPEDFA |
| | | TYYCQQFTSSPFTFGQGTKLEIK |
| SEQ ID NO: 35 | DNA VL | ATAACCAGCTGACCCAGTCTCCTTCCAGCCTGTCTGCTTC |
| | | TGTGGGCGACAGAGTGACAATTACCTGCCGGGCCTCCTC |
| | | CTCCGTGAACTACATCTACTGGTATCAGCAGAAGCCCGG |
| | | CAAGGCCCCTAAGCTGCTGATCTACTACACCTCCAATCT |
| | | GGCCCCTGGCGTGCCCTCTAGATTTTCCGGATCTGGCTCC |
| | | GGCAACGACTATACCCTGACAATCTCCAGCCTGCAGCCT |
| | | GAGGACTTCGCCACCTACTACTGCCAGCAGTTCACCTCC |
| | | TCTCCATTCACCTTTGGCCAGGGCACCAAGCTGGAAATC |
| | | AAA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 28 | VL | ENVLTQSPATLSVSPGERATLSCRASSSVNYIYWYQQKPGQ |
| | | APRLLIYYTSNLAPGIPARFSGSGSGNEYTLTISSLQSEDFAV |
| | | YYCQQFTSSPFTFGQGTKLEIK |
| SEQ ID NO: 36 | DNA VL | GAGAATGTGCTGACCCAGTCTCCTGCCACACTGTCTGTT |
| | | AGCCCTGGCGAGAGAGCTACCCTGAGCTGCAGAGCCTCT |
| | | TCCTCCGTGAACTACATCTACTGGTATCAGCAGAAGCCC |
| | | GGCCAGGCTCCTAGACTGCTGATCTACTACACCTCCAAT |
| | | CTGGCCCCTGGCATCCCTGCCAGATTTTCCGGATCTGGCT |
| | | CCGGCAACGAGTATACCCTGACCATCTCCAGCCTGCAGT |
| | | CCGAGGACTTTGCTGTGTACTATTGCCAGCAGTTCACAA |
| | | GCAGCCCTTTCACCTTTGGCCAGGGCACCAAGCTGGAAA |
| | | TCAAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 29 | VL | QNVLTQPPSASGTPGQRVTISCRASSSVNYIYWYQQLPGTA |
| | | PKLLIYYTSNLAPGVPDRFSGSGSGNSYSLAISGLRSEDEAD |
| | | YYCQQFTSSPFTFGTGTKVTVL |
| SEQ ID NO: 37 | DNA VL | CAGAATGTGCTGACCCAACCTCCTTCCGCCTCTGGCACA |
| | | CCTGGACAGAGAGTGACAATCTCCTGCCGGGCCTCCTCC |
| | | TCCGTGAACTACATCTACTGGTATCAGCAGCTGCCCGGC |
| | | ACCGCTCCTAAACTGCTGATCTACTACACCTCCAATCTG |
| | | GCCCCTGGCGTGCCCGATAGATTTTCCGGATCTGGCTCC |
| | | GGCAACTCCTACAGCCTGGCTATCTCTGGCCTGAGATCT |
| | | GAGGACGAGGCCGACTACTACTGCCAGCAGTTCACCTCC |
| | | TCTCCATTCACCTTTGGCACCGGCACCAAAGTGACAGTT |
| | | CTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 30 | VL | SNELTQPPSVSVSPGQTARITCRASSSVNYIYWYQQKSGQA |
| | | PVLVIYYTSNLAPGIPERFSGSGSGNMYTLTISGAQVEDEAD |
| | | YYCQQFTSSPFTFGTGTKVTVL |
| SEQ ID NO: 38 | DNA VL | TCTAATGAGCTGACCCAGCCTCCTTCCGTGTCCGTGTCTC |
| | | CTGGACAGACCGCCAGAATTACCTGCCGGGCCTCCTCCT |
| | | CCGTGAACTACATCTACTGGTATCAGCAGAAGTCCGGCC |
| | | AGGCTCCTGTGCTCGTGATCTACTACACCTCCAATCTGGC |
| | | CCCTGGCATCCCTGAGAGATTCTCCGGATCTGGCTCCGG |
| | | CAACATGTACACCCTGACCATCTCTGGCGCCCAGGTGGA |
| | | AGATGAGGCCGACTACTACTGCCAGCAGTTCACCTCCTC |
| | | TCCATTCACCTTTGGCACCGGCACCAAAGTGACAGTTCT |
| | | T |

Antibody B-H.1

| | | |
|---|---|---|
| SEQ ID NO: | Chain 1: Fc only | METDTLLLWVLLLWVPGSTGDKTHTCPPCPAPELLGGPSV |
| 3280 | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPCREEMTK |
| | | NQVSLWCLVKGFYPSDIAVEWESNGQPENNYKTTPPVLDS |
| | | DGSFFLYSKLTVDKSRWQQGNVFSCSVMHEALHNRFTQKS |
| | | LSLSPGK |
| SEQ ID NO: | Chain 2: humanized | METDTLLLWVLLLWVPGSTGEVQLVESGGGLVQPGGSLRL |
| 3281 | B-H scFv | SCAASGFTFSNFGMHWVRQAPGKGLEWVSYISSGSSTIYYA |
| | | DTLKGRFTISRDNSKNTLYLQMNSLRAEDTAVYYCARRGE |
| | | GAMDYWGQGTTVTVSSGGGGSGGGGSGGGGSGGGGSDN |
| | | QLTQSPSFLSASVGDRVTITCRASSSVNYIYWYQQKPGKAP |
| | | KLLIYYTSNLAPGVPSRFSGSGSGNEYTLTISSLQPEDFATY |
| | | YCQQFTSSPFTFGQGTKLEIKGGGGSDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNG |
| | | KEYKCKVSNKALPAPIEKTISKAKGQPREPQVCTLPPSREE |
| | | MTKNQVSLSCAVKGFYPSDIAVEWESNGQPENNYKTTPPV |
| | | LDSDGSFFLVSKLTVDKSRWQQGNVFSCSVMHEALHNHYT |
| | | QKSLSLSPGKGGGGSGGGGSGLNDIFEAQKIEWHE |
| SEQ ID NO: | scFv | EVQLVESGGGLVQPGGSLRLSCAASGFTFSNFGMHWVRQA |
| 3642 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSFLSASVGDRVTITCR |
| | | ASSSVNYIYWYQQKPGKAPKLLIYYTSNLAPGVPSRFSGSG |
| | | SGNEYTLTISSLQPEDFATYYCQQFTSSPFTFGQGTKLEIK |

Antibody B-H.2

| | | |
|---|---|---|
| SEQ ID NO: | scFv | EVQLVESGGGLVQPGGSLRLSCAASGFTFSNFGMHWVRQA |
| 1338 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSSLSASVGDRVTITCR |
| | | ASSSVNYIYWYQQKPGKAPKLLIYYTSNLAPGVPSRFSGSG |
| | | SGNDYTLTISSLQPEDFATYYCQQFTSSPFTFGQGTKLEIK |

Antibody B-H.3

| | | |
|---|---|---|
| SEQ ID NO: | scFv | EVQLVESGGGLVQPGGSLRLSCAASQFTFSNFGMHWVRQA |
| 1339 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSSNELTQPPSVSVSPGQTARITCRA |
| | | SSSVNYIYWYQQKSGQAPVLVIYYTSNLAPGIPERFSGSGSG |
| | | NMYTLTISGAQVEDEADYYCQQFTSSPFTFGTGTKVTVL |

Antibody B-H.4

| | | |
|---|---|---|
| SEQ ID NO: | scFv | QVQLVESGGGVVQPGRSLRLSCAASGFTFSNFGMHWVRQ |
| 1340 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSFLSASVGDRVTI |
| | | TCRASSSVNYIYWYQQKPGKAPKLLIYYTSNLAPGVPSRFS |
| | | GSGSGNEYTLTISSLQPEDFATYYCQQFTSSPFTFGQGTKLEI |
| | | K |

Antibody B-H.5

| | | |
|---|---|---|
| SEQ ID NO: | scFv | QVQLVESGGGVVQPGRSLRLSCAASGFTFSNFGMHWVRQ |
| 1341 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSSLSASVGDRVTI |
| | | TCRASSSVNYIYWYQQKPGKAPKLLIYYTSNLAPGVPSRFS |
| | | GSGSGNDYTLTISSLQPEDFATYYCQQFTSSPFTFGQGTKLE |
| | | IK |

Antibody B-H.6

| | | |
|---|---|---|
| SEQ ID NO: | scFv | QVQLVESGGGVVQPGRSLRLSCAASGFTFSNFGMHWVRQ |
| 1342 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSSNELTQPPSVSVSPGQTARITC |
| | | RASSSVNYIYWYQQKSGQAPVLVIYYTSNLAPGIPERFSGS |
| | | GSGNMYTLTISGAQVEDEADYYCQQFTSSPFTFGTGTKVTV |
| | | L |

TABLE 3
Constant region amino acid sequences of human IgG heavy chains and human kappa light chain

| | | |
|---|---|---|
| Human kappa | LC | RTVAAPSVFI FPPSDEQLKS GTASVVCLLN NFYPREAKVQ WKVDNALQSG |
| constant region | | NSQESVTEQD SKDSTYSLSS TLTLSKADYE KHKVYACEVT HQGLSSPVTK |
| SEQ ID NO: 39 | | SFNRGEC |
| Human | LC | RTVAAPSVFIFPPSDEQLKSGTASVVCLLNNFYPREAKVQWKVDNALQSGNSQ |
| Immunoglobulin | | ESVTEQDSKDSTYSLSSTLTLSKADYEKHKVYACEVTHQGLSSPVTKSFNRGE |
| kappa constant C | | C |
| region | | |
| SEQ ID NO: 3644 | | |
| IgG4 (S228P) | HC | ASTKGPSVFPLAPCSRSTSESTAALGCLVKDYFPEPVTVSWNSGALTSGVHTFP |
| mutant constant | | AVLQSSGLYSLSSVVTVPSSSLGTKTYTCNVDHKPSNTKVDKRVESKYGPPCPP |
| region (EU | | CPAPEFLGGPSVFLFPPKPKDTLMISRTPEVTCVVVDVSQEDPEVQFNWYVDG |
| Numbering) | | VEVHNAKTKPREEQFNSTYRVVSVLTVLHQDWLNGKEYKCKVSNKGLPSSIE |
| SEQ ID NO: 40 | | KTISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQ |
| | | KSLSLSLG |
| IgG1 wild type | HC | ASTKGPSVFPLAPSSKSTSGGTAALGCLVKDYFPEPVTVSWNSGALTSGVHTFP |
| SEQ ID NO: 41 | | AVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgG1 (N297A) | HC | ASTKGPSVFPLAPSSKSTSGGTAALGCLVKDYFPEPVTVSWNSGALTSGVHTFP |
| mutant constant | | AVLQSSGLYSLSSVVTVPSSSLGTQTYICNVNHKPSNTKVDKRVEPKSCDKTHT |
| region (EU | | CPPCPAPELLGGPSVFLFPPKPKDTLMISRTPEVTCVVVDVSHEDPEVKENWYV |
| Numbering) | | DGVEVHNAKTKPREEQYASTYRVVSVLTVLHQDWLNGKEYKCKVSNKALPA |
| SEQ ID NO: 42 | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgM constant | HC | GSASAPTLFPLVSCENSPSDTSSVAVGCLAQDFLPDSITFSWKYKNNSDISSTRG |
| delta CDC | | FPSVLRGGKYAATSQVLLPSKDVMQGTDEHVVCKVQHPNGNKEKNVPLPVIA |
| (P311A, P313S) | | ELPPKVSVFVPPRDGFFGNPRKSKLICQATGFSPRQIQVSWLREGKQVGSGVTT |
| SEQ ID NO: 73 | | DQVQAEAKESGPTTYKVTSTLTIKESDWLGQSMFTCRVDHRGLTFQQNASSM |
| | | CVPDQDTAIRVFAIPPSFASIFLTKSTKLTCLVTDLTTYDSVTISWTRQNGEAVK |
| | | THTNISESHPNATFSAVGEASICEDDWNSGERFTCTVTHTDLASSLKQTISRPKG |
| | | VALHRPDVYLLPPAREQLNLRESATITCLVTGFSPADVFVQWMQRGQPLSPEK |
| | | YVTSAPMPEPQAPGRYFAHSILTVSEEEWNTGETYTCVVAHEALPNRVTERTV |
| | | DKSTGKPTLYNVSLVMSDTAGTCY |
| IgGA1 | HC | ASPTSPKVFPLSLCSTQPDGNVVIACLVQGFFPQEPLSVTWSESGQGVTARNFPP |
| SEQ ID NO: 74 | | SQDASGDLYTTSSQLTLPATQCLAGKSVTCHVKHYTNPSQDVTVPCPVPSTPPT |
| | | PSPSTPPTPSPSCCHPRLSLHRPALEDLLLGSEANLTCTLTGLRDASGVTFTWTP |
| | | SSGKSAVQGPPERDLCGCYSVSSVLPGCAEPWNHGKTFTCTAAYPESKTPLTA |
| | | TLSKSGNTFRPEVHLLPPPSEELALNELVTLTCLARGFSPKDVLVRWLQGSQEL |
| | | PREKYLTWASRQEPSQGTTTFAVTSILRVAAEDWKKGDTFSCMVGHEALPLAF |
| | | TQKTIDRLAGKPTHVNVSVVMAEVDGTCY |
| IgGA2 | HC | ASPTSPKVFPLSLDSTPQDGNVVVACLVQGFFPQEPLSVTWSESGQNVTARNFP |
| SEQ ID NO: 75 | | PSQDASGDLYTTSSQLTLPATQCPDGKSVTCHVKHYTNSSQDVTVPCRVPPPPP |
| | | CCHPRLSLHRPALEDLLLGSEANLTCTLTGLRDASGATFTWTPSSGKSAVQGPP |
| | | ERDLCGCYSVSSVLPGCAQPWNHGETFTCTAAHPELKTPLTANITKSQNTFRPE |
| | | VHLLPPPSEELALNELVTLTCLARGFSPKDVLVRWLQGSQELPREKYLTWASR |
| | | QEPSQGTTTYAVTSILRVAAEDWKKGETFSCMVGHEALPLAFTQKTIDRMAG |
| | | KPTHINVSVVMAEADGTCY |
| Human Ig_J chain | HC | MKNHLLFWGVLAVFIKAVHVKAQEDERIVLVDNKCKCARITSRIIRSSEDPNED |
| SEQ ID NO: 76 | | IVERNIRIIVPLNNRENISDPTSPLRTRFVYHLSDLCKKCDPTEVELDNQIVTATQ |
| | | SNICDEDSATETCYTYDRNKCYTAVVPLVYGGETKMVETALTPDACYPD |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3645 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3646 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGKEPEA |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKRVEPKSCDKTHT |
| heavy chain | | CPPCPAPELLGGPSVFLFPPKPKDTLMISRTPEVTCVVVDVSHEDPEVKFNWYV |
| SEQ ID NO: 3647 | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human Fc Knob | HC | DKTHTCPPCPAPELLGGPSVFLFPPKPKDTLMISRTPEVTCVVVDVSHEDPEVK |
| cys N297A | | FNWYVDGVEVHNAKTKPREEQYASTYRVVSVLTVLHQDWLNGKEYKCKVS |
| SEQ ID NO: 3648 | | NKALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMHE |
| | | ALHNHYTQKSLSLSPQK |
| Human IgG1 hole | HC | ASTKGPSVFPLAPSSKSTSGGTAALGCLVKDYFPEPVTVSWNSGALTSGVHTFP |
| cys N297A | | AVLQSSGLYSLSSVVTVPSSSLGTQTYICNVNHKPSNTKVDKRVEPKSCDKTHT |
| SEQ ID NO: 3649 | | CPPCPAPELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPA |
| | | PIEKTISKAKGQPREPQVCTLPPSREEMTKNQVSLSCAVKGFYPSDIAVEWESN |
| | | GQPENNYKTTPPVLDSDGSFFLVSKLTVDKSRWQQGNVFSCSVMHEALHNHY |
| | | TQKSLSLSPGK |

TABLE 4
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

TABLE 5
CRS grading

| | |
|---|---|
| Gr1 | Supportive care only |
| Gr2 | IV therapies +/− hospitalization. |
| Gr3 | Hypotension requiring IV fluids or low-dose |
| | vasoactives or hypoxemia requiring oxygen, CPAP, or |
| | BIPAP. |
| Gr4 | Hypotension requiring high-dose vasoactives |
| | or hypoxemia requiring mechanical ventilation. |
| Gr 5 | Death |

TABLE 6
CTCAE v 4.0 CRS grading scale

| CRS grade | Characteristics |
|---|---|
| Grade 1 | Mild; No infusion interruption; No intervention |
| Grade 2 | Infusion interruption indicated but responds |
| | promptly to symptomatic treatment (e.g., |
| | antihistamines, NSAIDS, narcotics, IV fluids); |
| | prophylactic medications indicated for <=24 hrs |
| Grade 3 | Prolonged (e.g., not rapidly responsive to |
| | symptomatic medications and/or brief interruption of |
| | infusion); recurrence of symptoms following |
| | initial improvement, hospitalization indicated for |
| | clinical sequelae (e.g., renal impairment, |
| | pulmonary infiltrates) |
| Grade 4 | Life threatening consequences; pressor or |
| | ventilator support |

TABLE 7
NCI CRS grading scale

| CRS grade | Characteristics |
|---|---|
| Grade 1 | Symptoms are not life threatening and require |
| | symptomatic treatment only; e.g., fever, nausea, |
| | fatigue, headache, myalgias, malaise |
| Grade 2 | Symptoms require and respond to moderate |
| | intervention; Oxygen requirement <40% of |
| | hypotension responsive to fluids or low |
| | dose pressors or Grade 2 organ toxicity |
| Grade 3 | Symptoms require and respond to aggressive |
| | intervention; Oxygen requirement >=40% or |
| | Hypotension requiring high dose or multiple |
| | pressors or grade 3 organ toxicity or grade 4 |
| | transaminitis |
| Grade 4 | Life threatening symptoms Requirement |
| | for ventilator support or Grade 4; organ toxicity |
| | (excluding transaminitis) |

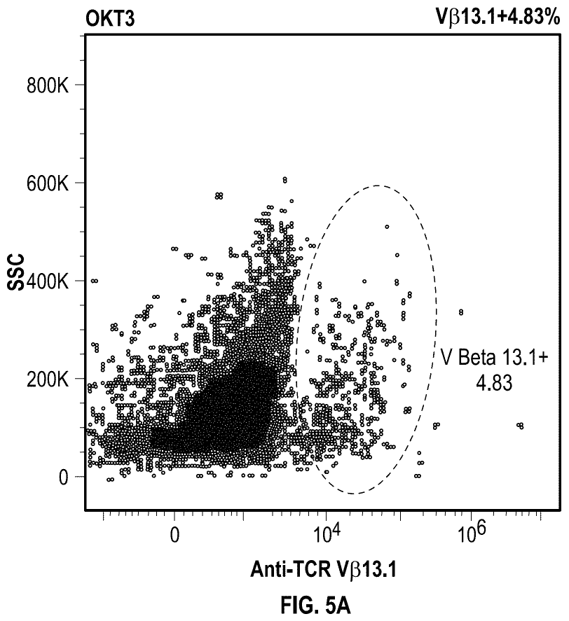

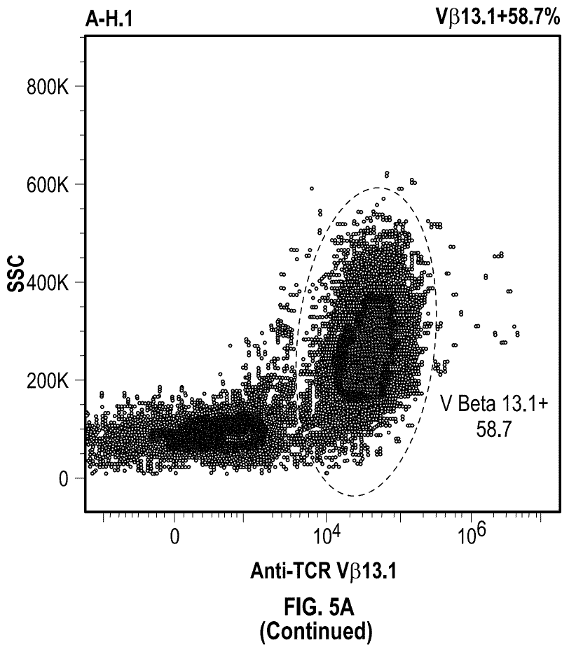

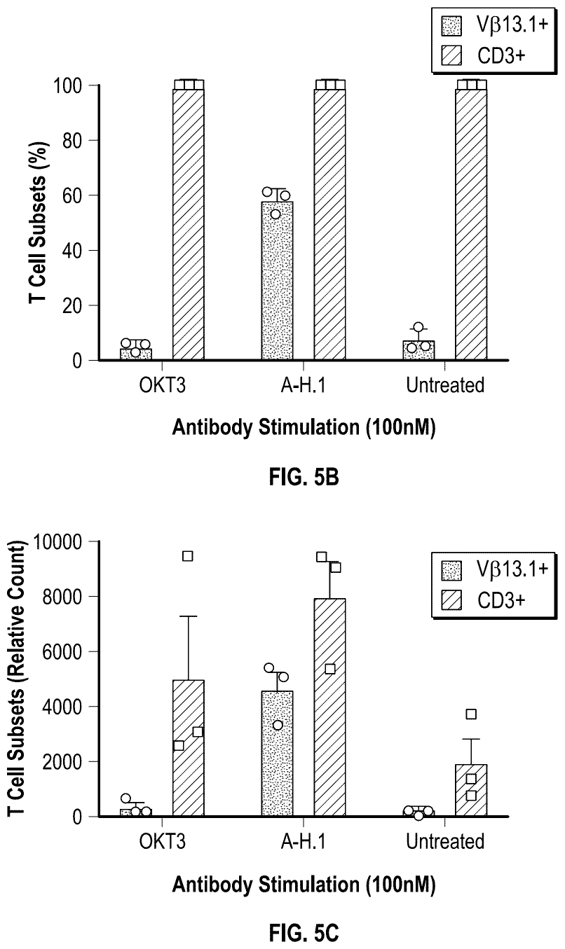

TABLE 8A
List of TCRβV subfamilies and subfamily members

| Reference | | |
|---|---|---|
| in FIG. 5 | Subfamily | Subfamily members |
| A | TCRβ V6 | TCRβ V6-4*01, TCRβ V6-4*02, |
| | Also referred | TCRβ V6-9*01, TCRβ V6-8*01, |
| | to as: | TCRβ V6-5*01, TCRβ V6-6*02, |
| | TCR VB 13.1 | TCRβ V6-6*01, TCRβ V6-2*01, |
| | | TCRβ V6-3*01 or TCRβ V6-1*01. |
| B | TCRβ V10 | TCRβ V10-1*01, TCRβ V10-1*02, |
| | Also | TCRβ V10-3*01 or TCRβ V10-2*01 |
| | referred to as: | |
| | TCRβ V12 | |
| C | TCRβ V12 | TCRβ V12-4*01, TCRβ V12-3*01, |
| | Also referred | or TCRβ V12-5*01 |
| | to as: | |
| | TCRβ V8.1 | |
| D | TCRβ V5 | TCRβ V5-5*01, TCRβ V5-6*01, |
| | | TCRβ V5-4*01, TCRβ V5-8*01, |
| | | TCRβ V5-1*01 |
| E | TCRβ V7 | TCRβ V7-7*01, TCRβ V7-6*01, |
| | | TCRβ V7-8*02, TCRβ V7-4*01, |
| | | TCRβ V7-2*02, TCRβ V7-2*03, |
| | | TCRβ V7-2*01, TCRβ V7-3*01, |
| | | TCRβ V7-9*03, or TCRβ V7-9*01 |
| F | TCRß V11 | TCRβ V11-1*01, TCRβ V11-2*01 |
| | | or TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, |
| | | or TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 8B
Additional TCRβV subfamilies
Subfamily
TCRβ V1
TCRβ V17
TCRβ V21
TCRβ V23
TCRβ V26

TABLE 10A
Exemplary anti-TCRBV antibody molecules

| TRBV | TRBV | Reagents monoclonal antibodies | | |
|---|---|---|---|---|
| vene name | allele name | Clone name and Specificity | Company product | Isotype |
| TRBV2 | TRBV2*01 | IsMMU 546 (TRBV2) | Serotec V BETA 22 | Mouse IgG1 |
| | TRBV2*02 | | Coulter Vbeta22 | |
| | TRBV2*03 | | | |
| TRBV3-1 | TRBV3-1*01 | FIN9 (TRBV3-1) | Serotec Vbeta9 | Mouse IgG2a |
| | | ZOE | Coulter Vbeta9 | |
| | TRBV3-1*02 | AMKB1-2 (TRBV3-1) | BD Biosciences Vbeta9 | Mouse IgG1 |
| TRBV4-1 | TRBV4-1*01 | ZOE (TRBV4-1, TRBV4-2, | Serotec V BETA 7 | Mouse IgG2a |
| | | TRBV4-3) | Coulter Vbeta7 | |
| | TRBV4-1*02 | 3G5 (TRBV4-1) | Pierce Endogen V beta 7.1 | Mouse IgG2b |
| TRBV4-2 | TRBV4-2*01 | ZOE (TRBV4-1, TRBV4-2, | Serotec V BETA 7 | Mouse IgG2a |
| | TRBV4-2*02 | TRBV4-3) | Coulter Vbeta7 | |
| TRBV4-3 | TRBV4-3*01 | ZOE (TRBV4-1, TRBV4-2, | Serotec V BETA 7 | Mouse IgG2a |
| | TRBV4-3*02 | TRBV4-3) | Coulter Vbeta7 | |
| | TRBV4-3*03 | | | |
| | TRBV4-3*04 | ZIZOU4 (TRBV4-3) | Coulter Vbeta7.2 | Mouse IgG2a |
| TRBV5-1 | TRBV5-1*01 | IMMU157 (TRBV5-1) | Serotec Vbeta5.1 | Mouse IgG2a |
| | | | Coulter Vbeta5.1 | |
| | TRBV5-1*02 | LC4 (TRBV5-1) | Pierce Endogen V beta 5(c) | Mouse IgG1 |
| | | | BD Biosciences VbetaS(c) | |
| TRBV5-4 | TRBV5-4*01 | | | |
| | TRBV5-4*02 | | | |
| | TRBV5-4*03 | | | |
| | TRBV5-4*04 | | | |
| TRBV5-5 | TRBV5-5*01 | 3D11 (TRBV5-5) | Serotec VBETA5.3 | Mouse IgG1 |
| | | | Coulter Vbeta5.3 | |
| | TRBV5-5*02 | 1C1 (TRBV5-5, TRBV5-6) | Pierce Endogen V beta 5(a) | Mouse IgG1 |
| | | | BD Biosciences Vbeta5(a) | |
| | TRBV5-5*03 | W112 (TRBV5-5) | Pierce Endogen V beta 5(b) | Mouse IgG1 |
| | | MH3-2 (TRBV5-5, | Serotec V beta 5.2/5.3 | |
| | | | BD Biosciences Vbeta5(b) | |
| | | TRBV5-6) | BD Biosciences VbetaS | Mouse IgG2a |
| | | 4H11 (TM27) as disclosed | | |
| | | in U.S. Patent 5,861,155 | | |
| TRBV5-6 | TRBV5-6*01 | 36213 (TRBV5-6) | Serotec Vbeta5.2 | Mouse IgG1 |
| | | 1C1 (TRBV5-5, TRBV5-6) | BD Biosciences Vbeta5(a) | Mouse IgG1 |
| | | MH3-2 (TRBV5-S, | BD Biosciences Vbeta | Mouse IgG2a |
| | | TRBV5-6) | | |
| TRBV5-8 | TRBV5-8*01 | | | |
| | TRBV5-8*02 | | | |
| TRBV6-1 | TRBV6-1*01 | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-2 | TRBV6-2*01 | H132 | Coulter Vbeta13.2 | Mouse IgG1 |
| TRBV6-3 | TRBV6-3*01 | | | |
| TRBV6-4 | TRBV6-4*01 | | | |
| | TRBV6-4*02 | | | |
| TRBV6-5 | TRBV6-5*01 | IMMU 222 (TRBV6-5, | Serotec V BETA 13.1 | Mouse IgG2b |
| | | TRBV6-6 and TRBV6-9) | Coulter Vbeta13.1 | |
| | | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-6 | TRBV6-6*01 | JU-74 (TRBV6-6) | Serotec Vbeta13.6 | Mouse IgG1 |
| | TRBV6-6*02 | JU74.3 (TRBV6-6) | Coulter Vbeta13.6 | |
| | TRBV6-6*03 | IMMU 222 (TRBV6-5, | Serotec V BETA 13.1 | Mouse IgG2b |
| | TRBV6-6*04 | TRBV6-6 and TRBV6-9) | Coulter Vbeta13.1 | |
| | TRBV6-6*05 | | | |
| TRBV6-8 | TRBV6-8*01 | | | |
| TRBV6-9 | TRBV6-9*01 | IMMU 222 (TRBV6-5, | Serotec V BETA 13.1 | Mouse IgG2b |
| | | TRBV6-6 and TRBV6-9) | Coulter Vbeta13.1 | |
| TRBV7-2 | TRBV7-2*01 | OT145 (TRBV7-2) | Pierce Endogen V beta 6.7 | Mouse IgG1 |
| | TRBV7-2*02 | | BD Biosciences Vbeta6.7 | |
| | TRBV7-2*03 | | | |
| | TRBV7-2*04 | | | |
| TRBV7-3 | TRBV7-3*01 | | | |
| | TRBV7-3*04 | | | |
| | TRBV7-3*05 | | | |
| TRBV7-4 | TRBV7-4*01 | | | |
| TRBV7-6 | TRBV7-6*01 | | | |
| | TRBV7-6*02 | | | |
| TRBV7-7 | TRBV7-7*01 | | | |
| | TRBV7-7*02 | | | |
| TRBV7-8 | TRBV7-8*01 | | | |
| | TRBV7-8*02 | | | |
| | TRBV7-8*03 | | | |
| TRBV7-9 | TRBV7-9*01 | | | |
| | TRBV7-9*02 | | | |
| | TRBV7-9*03 | | | |
| | TRVB7-9*04 | | | |
| | TRBV7-9*05 | | | |
| | TRBV7-9*06 | | | |
| | TRBV7-9*07 | | | |
| TRBV9 | TRBV9*01 | BL37.2 (TRBV9) | Serotec Vbetal | Rat IgG1 |
| | TRBV9*02 | | Coulter Vbetal | |
| | TRBV9*03 | | | |
| TRBV10-1 | TRBV10-1*01 | S511 (TRBV10-1, | Pierce Endogen V beta 12 | Mouse IgG2b |
| | TRBV10-1*02 | TRBV10-2, TRBV10-3) | BD Biosciences Vbeta12 | |
| TRBV10-2 | TRBV10-2*01 | | | |
| | TRBV10-2*02 | | | |
| TRBV10-3 | TRBV10-3*01 | VER2.32.1 (TRBV10-3) | Serotec Vbeta12 | Mouse IgG2a |
| | TRBV10-3*02 | S511 (TRBV10-1, | Coulter Vbeta12 | |
| | TRBV10-3*03 | TRBV10-2, TRBV10-3) | Pierce Endogen V beta 12 | Mouse IgG2b |
| | TRBV10-3*04 | | BD Biosciences Vbeta12 | |
| TRBV11-1 | TRBV11-1*01 | | | |
| TRBV11-2 | TRBV11-2*01 | IG125 (TRBV11-2) | Serotec Vbeta21.3 | Mouse IgG2a |
| | TRBV11-2*02 | | Coulter Vbeta21.3 | |
| | TRBV11-2*03 | | | |
| TRBV11-3 | TRBV11-3*01 | | | |
| | TRBV11-3*02 | | | |
| | TRBV11-3*03 | | | |
| | TRBV11-3*04 | | | |
| TRBV12-3 | TRBV12-3*01 | 56C5 (TRBV12-3, | Serotec Vbeta8.1/8.2 | Mouse IgG2a |
| | | TRBV12-4) | Coulter Vbeta8 | |
| TRBV12-4 | TRBV12-4*01 | 56C5.2 (TRBV12-3, | Pierce Endogen V beta 8(a) | Mouse IgG2b |
| | TRBV12-4*02 | TRBV12-4) | BD Biosciences Vbeta8 | |
| | | 16G8 (TRBV12-3, | Pierce Endogen V beta 8(b) | Mouse IgG2a |
| | | TRBV12-4) | BD Biosciences | Mouse IgG2b |
| | | MX-6 (TRBV12-3, | Vbeta8 | |
| | | TRBV12-4) | | |
| | | JR2 (TRBV12-3, TRBV12- | | |
| | | 4, TRBV12-5) | | |
| TRBV12-5 | TRBV12-5*01 | JR2(TRBV12-3, TRBV12- | BD Biosciences Vbeta8 | Mouse IgG2b |
| | | 4, TRBV12-5) | | |
| TRBV13 | TRBV13*01 | AF-23 (TRBV13) | Serotec Vbeta23 | Mouse IgG1 |
| | TRBV13*02 | AF23 (TRBV13) | Coulter Vbeta23 | |
| | | AHUT7 (Vbeta23) | BD Biosciences Vbeta23 | |
| TRBV14 | TRBV14*01 | TAMAYA1.2 (TRBV14) | Serotec Vbeta16 | Mouse IgG1 |
| | TRBV14*02 | | Coulter Vbeta16 | |
| TRBV15 | TRBV15*01 | | | |
| | TRBV15*02 | | | |
| | TRBV15*03 | | | |
| TRBV16 | TRBV16*01 | | | |
| | TRBV16*03 | | | |
| TRBV18 | TRBV18*01 | BA62 (TRBV18) | Serotec V BETA 18 | Mouse IgG1 |
| | | BA62.6 (TRBV18) | Coulter Vbeta18 | |
| TRBV 19 | TRBV19*01 | C1 (TRBV19) | Pierce Endogen V beta 17 | Mouse IgG1 |
| | | E17.5F3 (TRBV19) | BD Biosciences Vbeta 17 | |
| | TRBV19*02 | E17.5F3.15.13 (TRBV19) | Serotec Vbetal7 | Mouse IgG1 |
| | TRBV19*03 | | Coulter Vbeta17 | |
| TRBV20-1 | TRBV20-1*01 | MPB2DS (TRBV20-1) | Serotec VBETA2 | Mouse IgG1 |
| | TRBV20-1*02 | | Coulter Vbeta2 | |
| | TRBV20-1*03 | | | |
| | TRBV20-1*04 | | | |
| | TRBV20-1*05 | | | |
| | TRBV20-1*06 | | | |
| | TRBV20-1*07 | | | |
| TRBV24-1 | TRBV24-1*01 | | | |
| TRBV25-1 | TRBV25-1*01 | C21 (TRBV25-1) | Serotec V BETA 11 | Mouse IgG2a |
| | | | Coulter Vbetall | |
| TRBV27 | TRBV27*01 | CAS1.1.3 (TRBV27) | Serotec Vbeta14 | Mouse IgG1 |
| | | | Coulter Vbeta14 | |
| TRBV28 | TRBV28*01 | CH92 (TRBV28) | Serotec Vbeta3 | Mouse IgM |
| | | | Coulter Vbeta3 | |
| | | 8F10 (TRBV28) | Pierce Endogen V beta 3.1 | Mouse IgG1 |
| | | JOVI-3 (TRBV28) | BD Biosciences Vbeta3 | Mouse IgG2a |
| TRBV29-1 | TRBV29-1*01 | WJF24 | Coulter Vbeta4 | Rat IgM |
| | TRBV29-1*02 | | | |
| | TRBV29-1*03 | | | |
| TRBV30 | TRBV30*01 | ELL1.4 (TRBV30) | Serotec Vbeta20 | Mouse IgG1 |
| | TRBV30*02 | | Coulter Vbeta20 | |
| | TRBV30*04 | | | |
| | TRBV30*05 | | | |

TABLE 10B
Amino acid sequences for anti TCRβ V5 antibodies. Amino acid and nucleotide sequences for
murine and humanized antibody molecules which bind to TCRVB 5 (e.g., TCRVB 5-5 or TCRVB 5-6).
The amino acid the heavy and light chain CDRs, and the amino acid and nucleotide sequences
of the heavy and light chain variable regions, and the heavy and light chains are shown.
Marine antibody C, also referred to as 4H11

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3 (Combined) | QQYSKLPRT |
| SEQ ID NO: 232 | VH | DIQMTQTTSSLSASLGDRVTISCSASQGISNYLNWYQQK |
| | | PDGTVKLLIYYTSSLHSGVPSRFSGSGSGTDYSLTISNLE |
| | | PEDIATYYCQQYSKLPRTFGGGTKVEIK |
| SEQ ID NO: 233 | VL | QVQLKESGPGLVAPSQSLSITCTVSGFSLTAYGVNWVR |
| | | QPPGKGLEWLGMIWGDGNTDYNSALKSRLSISKDNSKS |
| | | QVFLKMNSLQTDDTARYYCARDRVTATLYAMDYWGQ |
| | | GTSVTVSS |

Humanized antibody C
C-H-1 antibody

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3 (Combined) | QQYSKLPRT |
| SEQ ID NO: 1324 | VL | DIQMTQSPSSLSASVGDRVTITCSASQGISNYLNWYQQTP |
| | | GKAPKLLIYYTSSLHSGVPSRFSGSGSGTDYTFTISSLQPE |
| | | DIATYYCQQYSKLPRTFGQGTKLQIT |
| SEQ ID NO: 1325 | VH | QVQLQESGPGLVRPSQTLSLTCTVSGFSLTAYGVNWVR |
| | | QPPGRGLEWLGMIWGDGNTDYNSALKSRVTMLKDTSK |
| | | NQFSLRLSSVTAADTAVYYCARDRVTATLYAMDYW |
| | | GQGSLVTVSS |

Humanized antibody C Variable light chain (VL)

| | | | |
|---|---|---|---|
| SEQ ID NO: 3000 | VL | C-H-VL.1 | DIQMTQSPSFLSASVGDRVTITCSASQGISNYLNWYQQK |
| | | | PGKAVKLLIYYTSSLHSGVPSRFSGSGSGTEYTLTISSLQ |
| | | | PEDFATYYCQQYSKLPRTFGGGTKVEIK |
| SEQ ID NO: 3001 | VL | C-H-VL.2 | DIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3002 | VL | C-H-VL.3 | DIQMTQSPSSLSASVGDRVTITCSASQGISNYLNWYQQK |
| | | | PGKVVKLLIYYTSSLHSGVPSRFSGSGSGTDYTLTISSLQ |
| | | | PEDVATYYCQQYSKLPRTFGGGTKVEIK |
| SEQ ID NO: 3003 | VL | C-H-VL.4 | DIQMTQSPSSLSASVGDRVTITCSASQGISNYLNWYQQK |
| | | | PGQAVKLLIYYTSSLHSGVPSRFSGSGSGTDYTLTISSLQ |
| | | | PEDVATYYCQQYSKLPRTFGGGTKVEIK |
| SEQ ID NO: 3004 | VL | C-H-VL.5 | DIQMTQSPSSLSASVGDRVTITCSASQGISNYLNWYQQK |
| | | | PGKAVKLLIYYTSSLHSGVPSRFSGSGSGTDYTFTISSLQ |
| | | | PEDIATYYCQQYSKLPRTFGGGTKVEIK |
| SEQ ID NO: 3005 | VL | C-H-VL.6 | DIQMTQSPSSLSASVGDRVTITCSASQGISNYLNWYQQK |
| | | | PGKTVKLLIYYTSSLHSGIPSRFSGSGSGTDYTLTIRSLQP |
| | | | EDFATYYCQQYSKLPRTFGGGTKVEIK |
| SEQ ID NO: 3006 | VL | C-H-VL.7 | AIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3007 | VL | C-H-VL.8 | DIQMTQSPSSVSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3008 | VL | C-H-VL.9 | DIQMTQSPSSLSASVGDRVTITCSASQGISNYLNWYQQK |
| | | | PGKAVKRLIYYTSSLHSGVPSRFSGSGSGTEYTLTISNLQ |
| | | | PEDFATYYCQQYSKLPRTFGGGTKVEIK |
| SEQ ID NO: 3009 | VL | C-H-VL.10 | AIRMTQSPFSLSASVGDRVTITCSASQGISNYLNWYQQK |
| | | | PAKAVKLFIYYTSSLHSGVPSRFSGSGSGTDYTLTISSLQ |
| | | | PEDFATYYCQQYSKLPRTFGGGTKVEIK |
| SEQ ID NO: 3010 | VL | C-H-VL.11 | DIQMTQSPSSLSASVGDRVTITCSASQGISNYLNWYQQK |
| | | | PGKAVKRLIYYTSSLHSGVPSRFSGSGSGTEYTLTISSLQ |
| | | | PEDFATYYCQQYSKLPRTFGGGTKVEIK |
| SEQ ID NO: 3011 | VL | C-H-VL.12 | DIQMTQSPSTLSASVGDRVTITCSASQGISNYLNWYQQK |
| | | | PGKAVKLLIYYTSSLHSGVPSRFSGSGSGTEYTLTISSLQ |
| | | | PDDFATYYCQQYSKLPRTFGGGTKVEIK |
| SEQ ID NO:3012 | VL | C-H-VL.13 | DIQMTQSPSSLSASVGDRVTITCSASQGISNYLNWYQQK |
| | | | PGKAVKSLIYYTSSLHSGVPSRFSGSGSGTDYTLTISSLQ |
| | | | PEDFATYYCQQYSKLPRTFGGGTKVEIK |
| SEQ ID NO: 3013 | VL | C-H-VL.14 | DIQMTQSPSSLSASVGDRVTITCSASQGISNYLNWYQQK |
| | | | PGKAVKSLIYYTSSLHSGVPSKFSGSGSGTDYTLTISSLQ |
| | | | PEDFATYYCQQYSKLPRTFGGGTKVEIK |
| SEQ ID NO: 3014 | VL | C-H-VL.15 | DIQMTQSPSSLSASVGDRVTITCSASQGISNYLNWYQQK |
| | | | PEKAVKSLIYYTSSLHSGVPSRFSGSGSGTDYTLTISSLQ |
| | | | PEDFATYYCQQYSKLPRTFGGGTKVEIK |
| SEQ ID NO: 3015 | VL | C-H-VL.16 | DIQMTQSPSAMSASVGDRVTITCSASQGISNYLNWYQQ |
| | | | KPGKVVKRLIYYTSSLHSGVPSRFSGSGSGTEYTLTISSL |
| | | | QPEDFATYYCQQYSKLPRTFGGGTKVEIK |
| SEQ ID NO: 3016 | VL | C-H-VL.17 | DIVMTQSPDSLAVSLGERATINCSASQGISNYLNWYQQ |
| | | | KPGQPVKLLIYYTSSLHSGVPDRFSGSGSGTDYTLTISSL |
| | | | QAEDVAVYYCQQYSKLPRTFGGGTKVEIK |
| SEQ ID NO: 3017 | VL | C-H-VL.18 | EIVMTQSPGTLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3018 | VL | C-H-VL.19 | EIVMTQSPPTLSLSPGERVTLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3019 | VL | C-H-VL.20 | EIVMTQSPPTLSLSPGERVTLSCSASQGISNYLNWYQQK |
| | | | PGQAVKLLIYYTSSLHSSIPARFSGSGSGTDYTLTISSLQP |
| | | | EDFAVYYCQQYSKLPRTFGGGTKVEIK |
| SEQ ID NO: 3020 | VL | C-H-VL.21 | EIVMTQSPATLSLSPGERATLSCSASQGISNYLNWYQQK |
| | | | PGQAVELLIYYTSSLHSGIPARFSGSGSGTDYTLTISSLEP |
| | | | EDFAVYYCQQYSKLPRTFGGGTKVEIK |
| SEQ ID NO: 3021 | VL | C-H-VL.22 | EIVMTQSPATLSLSPGERATLSCSASQGISNYLNWYQQK |
| | | | PGQAVKLLIYYTSSLHSGIPARFSGSGSGTDYTLTISRLEP |
| | | | EDFAVYYCQQYSKLPRTFGGGTKVEIK |
| SEQ ID NO: 3022 | VL | C-H-VL.23 | EIVMTQSPATLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3023 | VL | C-H-VL.24 | EIVMTQSPATLSLSPGERATLSCSASQGISNYLNWYQQK |
| | | | PGLAVKLLIYYTSSLHSGIPDRFSGSGSGTDYTLTISRLEP |
| | | | EDFAVYYCQQYSKLPRTFGGGTKVEIK |
| SEQ ID NO: 3024 | VL | C-H-VL.25 | DIQMIQSPSFLSASVGDRVSIICSASQGISNYLNWYLQKP |
| | | | GKSVKLFIYYTSSLHSGVSSRFSGRGSGTDYTLTIISLKPE |
| | | | DFAAYYCQQYSKLPRTFGGGTKVEIK |
| SEQ ID NO: 3025 | VL | C-H-VL.26 | EIVMTQSPATLSLSPGERATLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3026 | VL | C-H-VL.27 | EIVMTQSPATLSLSPGERATLSCSASQGISNYLNWYQQK |
| | | | PGQAVKLLIYYTSSLHSGIPARFSGSGPGTDYTLTISSLEP |
| | | | EDFAVYYCQQYSKLPRTFGGGTKVEIK |
| SEQ ID NO: 3027 | VL | C-H-VL.28 | DIVMTQTPLSLSVTPGQPASISCSASQGISNYLNWYLQK |
| | | | PGQSVKLLIYYTSSLHSGVPDRFSGSGSGTDYTLKISRVE |
| | | | AEDVGVYYCQQYSKLPRTFGGGTKVEIK |
| SEQ ID NO: 3028 | VL | C-H-VL.29 | DIVMTQTPLSLSVTPGQPASISCSASQGISNYLNWYLQK |
| | | | PGQPVKLLIYYTSSLHSGVPDRFSGSGSGTDYTLKISRVE |
| | | | AEDVGVYYCQQYSKLPRTFGGGTKVEIK |
| SEQ ID NO: 3029 | VL | C-H-VL.30 | DIVMTQSPAFLSVTPGEKVTITCSASQGISNYLNWYQQK |
| | | | PDQAVKLLIYYTSSLHSGVPSRFSGSGSGTDYTFTISSLE |
| | | | AEDAATYYCQQYSKLPRTFGGGTKVEIK |
| SEQ ID NO: 3030 | VL | C-H-VL.31 | DIVMTQSPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3031 | VL | C-H-VL.32 | DIVMTQTPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3032 | VL | C-H-VL.33 | EIVMTQSPATLSVSPGERATLSCSASQGISNYLNWYQQK |
| | | | PGQAVKLLIYYTSSLHSGIPARFSGSGSGTEYTLTISILQS |
| | | | EDFAVYYCQQYSKLPRTFGGGTKVEIK |
| SEQ ID NO: 3033 | VL | C-H-VL.34 | EIVMTQSPATLSVSPGERATLSCSASQGISNYLNWYQQK |
| | | | PGQAVELLIYYTSSLHSGIPARFSGSGSGTEYTLTISSLQS |
| | | | EDFAVYYCQQYSKLPRTFGGGTKVEIK |
| SEQ ID NO: 3034 | VL | C-H-VL.35 | DIVMTQSPLSLPVTLGQPASISCSASQGISNYLNWYQQR |
| | | | PGQSVKRLIYYTSSLHSGVPDRFSGSGSGTDYTLKISRVE |
| | | | AEDVGVYYCQQYSKLPRTFGGGTKVEIK |
| SEQ ID NO: 3035 | VL | C-H-VL.36 | EITMTQSPAFMSATPGDKVNISCSASQGISNYLNWYQQ |
| | | | KPGEAVKFIIYYTSSLHSGIPPRFSGSGYGTDYTLTINNIE |
| | | | SEDAAYYYCQQYSKLPRTFGGGTKVEIK |
| SEQ ID NO: 3036 | VL | C-H-VL.37 | DIVMTQTPLSSPVTLGQPASISCSASQGISNYLNWYQQR |
| | | | PGQPVKLLIYYTSSLHSGVPDRFSGSGAGTDYTLKISRV |
| | | | EAEDVGVYYCQQYSKLPRTFGGGTKVEIK |
| SEQ ID NO: 3037 | VL | C-H-VL.38 | EIVMTQSPDFQSVTPKEKVTITCSASQGISNYLNWYQQK |
| | | | PDQSVKLLIYYTSSLHSGVPSRFSGSGSGTDYTLTINSLE |
| | | | AEDAATYYCQQYSKLPRTFGGGTKVEIK |
| SEQ ID NO: 3038 | VL | C-H-VL.39 | EIVMTQTPLSLSITPGEQASISCSASQGISNYLNWYLQKA |
| | | | RPVVKLLIYYTSSLHSGVPDRFSGSGSGTDYTLKISRVEA |
| | | | EDFGVYYCQQYSKLPRTFGGGTKVEIK |
| SEQ ID NO: 3039 | VL | C-H-VL.40 | EIVMTQTPLSLSITPGEQASMSCSASQGISNYLNWYLQK |
| | | | ARPVVKLLIYYTSSLHSGVPDRESGSGSGTDYTLKISRVE |
| | | | AEDFGVYYCQQYSKLPRTFGGGTKVEIK |

Humanized antibody C Variable HEAVY chain (VH)

| | | | |
|---|---|---|---|
| SEQ ID NO: 3040 | VH | C-H-VH.1 | QVTLKESGPVLVKPTETLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3041 | VH | C-H-VH.2 | QVTLKESGPALVKPTETLTLTCTVSGFSLTAYGVNWVR |
| | | | QPPGKALEWLGMIWGDGNTDYNSALKSRLIISKDNSKS |
| | | | QVVLTMTNMDPVDTATYYCARDRVTATLYAMDYWG |
| | | | QGTLVTVSS |
| SEQ ID NO: 3042 | VH | C-H-VH.3 | QVTLKESGPALVKPTQTLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3043 | VH | C-H-VH.4 | QVQLQESGPGLVKPSGTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3044 | VH | C-H-VH.5 | QVTLKESGPTLVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3045 | VH | C-H-VH.6 | QVTLKESGPALVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3046 | VH | C-H-VH.7 | QVQLQESGPGLVKPSQTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3047 | VH | C-H-VH.8 | QVQLQESGPGLVKPSETLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3048 | VH | C-H-VH.9 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3049 | VH | C-H-VH.10 | QVQLQESGPGLVKPSDTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3050 | VH | C-H-VH.11 | QVQLQESGPGLVKPSQTLSLTCTVSGFSLTAYGVNWVR |
| | | | QHPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3051 | VH | C-H-VH.12 | QVQLQESGPGLVKPSQTLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3052 | VH | C-H-VH.13 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3053 | VH | C-H-VH.14 | QVQLQESGPGLVKPSETLSLTCTVSGFSLTAYGVNWVR |
| | | | QPPGKGLEWLGMIWGDGNTDYNSALKSRLTISKDNSKS |
| | | | HVSLKLSSVTAADTAVYYCARDRVTATLYAMDYWGQ |
| | | | GTLVTVSS |
| SEQ ID NO: 3054 | VH | C-H-VH.15 | QVQLQESGPGLVKPSETLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3055 | VH | C-H-VH.16 | QVQLQESGPGLVKPSQTLSLTCAVYGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3056 | VH | C-H-VH.17 | RVQLQESGPGLVKPSETLSLTCTVSGFSLTAYGVNWVR |
| | | | QPPGKGLEWLGMIWGDGNTDYNSALKSRLTISKDNSKS |
| | | | QVPLKLSSVTAADTAVYYCARDRVTATLYAMDYWGQ |
| | | | GTLVTVSS |
| SEQ ID NO: 3057 | VH | C-H-VH.18 | QVQLQESGPGLVKPSQTLSLTCTVSGFSLTAYGVNWVR |
| | | | QHPGKGLEWLGMIWGDGNTDYNSALKSLLTISKDNSKS |
| | | | QVSLKLSSVTAADTAVYYCARDRVTATLYAMDYWGQ |
| | | | GTLVTVSS |
| SEQ ID NO: 3058 | VH | C-H-VH.19 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTALDTAVYYCARDRVTATLYAMDYWGQ |
| | | | GTLVTVSS |
| SEQ ID NO: 3059 | VH | C-H-VH.20 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3060 | VH | C-H-VH.21 | QVQLQESGSGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3061 | VH | C-H-VH.22 | EVQLVESGGGLVQPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3062 | VH | C-H-VH.23 | EVQLVESGGGLVQPGPSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3063 | VH | C-H-VH.24 | QVQLQESGSGLVKPSQTLSLTCAVSGFSLTAYGVNWVR |
| | | | QSPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3064 | VH | C-H-VH.25 | QVQLQESGPGLVKPSETLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3065 | VH | C-H-VH.26 | EVQLVESGGGLVKPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3066 | VH | C-H-VH.27 | QVQLQESGPGLVKPSETLSLTCAVYGFSLTAYGVNWVR |
| | | | QPPGKGLEWLGMIWGDGNTDYNSALKSRLTISKDNSKS |
| | | | QVYLKLSSVTAADTAVYYCARDRVTATLYAMDYWGQ |
| | | | GTLVTVSS |
| SEQ ID NO: 3067 | VH | C-H-VH.28 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTGVYYCARDRVTATLYAMDYWGQ |
| | | | GTLVTVSS |
| SEQ ID NO: 3068 | VH | C-H-VH.29 | EVQLVESGGGLVQPGGSLRLSCAVSGFSLTAYGVNWV |
| | | | RQAPGKGLEWLGMIWGDQNTDYNSALKSRLTISKDNS |
| | | | KSSVYLQMNSLKTEDTAVYYCARDRVTATLYAMDYW |
| | | | GQGTLVTVSS |
| SEQ ID NO: 3069 | VH | C-H-VH.30 | EVQLVESGGGLVKPGGSLRLSCAVSGFSLTAYGVNWV |
| | | | RQAPGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3070 | VH | C-H-VH.31 | QVQLQQSGPGLVKPSQTLSLTCAVSGFSLTAYGVNWVR |
| | | | QSPSRGLEWLGMIWGDGNTDYNSALKSRLTINKDNSKS |
| | | | QVSLQLNSVTPEDTAVYYCARDRVTATLYAMDYWGQ |
| | | | GTLVTVSS |
| SEQ ID NO: 3071 | VH | C-H-VH.32 | QVQLVESGGGLVQPGGSLRLSCSVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3072 | VH | C-H-VH.33 | QVQLQQWGAGLLKPSETLSLTCAVYGFSLTAYGVNWV |
| | | | RQPPGKGLEWLGMIWGDGNTDYNSALKSRLTISKDNSK |
| | | | SQVSLKLSSVTAADTAVYYCARDRVTATLYAMDYWG |
| | | | QGTLVTVSS |
| SEQ ID NO: 3073 | VH | C-H-VH.34 | QVQLVESGGGVVQPGRSLRLSCAVSGFSLTAYGVNWV |
| | | | RQAPGKGLEWLGMIWGDGNTDYNSALKSRLTISKDNST |
| | | | STVFLQMNSLRAEDTAVYYCARDRVTATLYAMDYWG |
| | | | QGTLVTVSS |
| SEQ ID NO: 3074 | VH | C-H-VH.35 | EVQLVESGGGLVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3075 | VH | C-H-VH.36 | EVQLVESGGGLVQPGGSLRLSCAVSQFSLTAYGVNWV |
| | | | RQAPGKGLEWLGMIWQDGNTDYNSALKSRLTISKDNA |
| | | | KSSVYLQMNSLRDEDTAVYYCARDRVTATLYAMDYW |
| | | | GQGTLVTVSS |
| SEQ ID NO: 3076 | VH | C-H-VH.37 | EVQLLESGGGLVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3077 | VH | C-H-VH.38 | QVQLVESGGGLVKPGGSLRLSCAVSGFSLTAYGVNWV |
| | | | RQAPGKGLEWLGMIWGDQNTDYNSALKSRLTISKDNA |
| | | | KSSVYLQMNSLRAEDTAVYYCARDRVTATLYAMDYW |
| | | | GQGTLVTVSS |
| SEQ ID NO: 3078 | VH | C-H-VH.39 | EVQLVESGGGLVQPGGSLKLSCAVSGFSLTAYGVNWV |
| | | | RQASGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3079 | VH | C-H-VH.40 | QVQLLESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3080 | VH | C-H-VH.41 | QVQLVESGGGVVQPGRSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3081 | VH | C-H-VH.42 | QVQLVESGGGVVQPGRSLRLSCAVSGFSLTAYGVNWV |
| | | | RQAPGKGLEWLGMIWGDGNTDYNSALKSRLTISKDNS |
| | | | KSRVYLQMNSLRAEDTAVYYCARDRVTATLYAMDYW |
| | | | GQGTLVTVSS |
| SEQ ID NO: 3082 | VH | C-H-VH.43 | QVQLVESGGGVVQPGRSLRLSCAVSGFSLTAYGVNWV |
| | | | RQAPGKGLEWLGMIWGDQNTDYNSALKSRLAISKDNS |
| | | | KSTVYLQMNSLRAEDTAVYYCARDRVTATLYAMDYW |
| | | | GQGTLVTVSS |
| SEQ ID NO: 3083 | VH | C-H-VH.44 | QVQLVESGGGVVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3084 | VH | C-H-VH.45 | EVQLVESQGGLVQPGGSLRLSCAVSGFSLTAYGVNWV |
| | | | RQAPGKGLEWLGMIWQDGNTDYNSALKSRLTISKDNA |
| | | | KSTVYLQMNSLRAEDTAVYYCARDRVTATLYAMDYW |
| | | | GQGTLVTVSS |
| SEQ ID NO: 3085 | VH | C-H-VH.46 | EVQLVESGGGLVQPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3086 | VH | C-H-VH.47 | EVQLVESGGVVVQPGGSLRLSCAVSGFSLTAYGVNWV |
| | | | RQAPGKGLEWLGMIWGDGNTDYNSALKSRLTISKDNS |
| | | | KSSVYLQMNSLRTEDTALYYCARDRVTATLYAMDYW |
| | | | GQGTLVTVSS |
| SEQ ID NO: 3087 | VH | C-H-VH.48 | EVQLVESGGGLVQPGGSLRLSCAVSGFSLTAYGVNWV |
| | | | RQAPGKGLEWLGMIWGDGNTDYNSALKSRLTISKHNS |
| | | | KSTVYLQMNSLRAEDTAVYYCARDRVTATLYAMDYW |
| | | | GQGTLVTVSS |
| SEQ ID NO: 3088 | VH | C-H-VH.49 | EVQLVESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3089 | VH | C-H-VH.50 | EVQLVESGGGLIQPGGSLRLSCAVSGFSLTAYGVNWVR |
| | | | QPPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |

TABLE 11
Amino acid sequences for anti TCRβ V5 antibodies. Amino acid and nucleotide sequences for
murine and humanized antibody molecules which bind to TCRVB 5 (e.g., TCRVB 5-5 or TCRVB 5-6).
The amino acid the heavy and light chain CDRs, and the amino acid and nucleotide sequences
of the heavy and light chain variable regions, and the heavy and light chains are shown.
Murine antibody E, also referred to as MH3-2

| | | |
|---|---|---|
| SEQ ID NO: 1298 | HC CDR1 (Kabat) | SSWMN |
| SEQ ID NO: 1299 | HC CDR2 (Kabat) | RIYPGDGDTKYNGKFKG |
| SEQ ID NO: 1300 | HC CDR3 (Kabat) | RGTGGWYFDV |
| SEQ ID NO: 1302 | HC CDR1 (Chothia) | GYAFSSS |
| SEQ ID NO: 1303 | HC CDR2 (Chothia) | YPGDGD |
| SEQ ID NO: 1301 | HC CDR3 (Chothia) | RGTGGWYFDV |
| SEQ ID NO: 1304 | HC CDR1 (Combined) | GYAFSSSWMN |
| SEQ ID NO: 1299 | HC CDR2 (Combined)) | RIYPGDGDTKYNGKFKG |
| SEQ ID NO: 1301 | HC CDR3 (Combined) | RGTGGWYFDV |
| SEQ ID NO: 1305 | LC CDR1 (Kabat) | RASESVDSSGNSFMH |
| SEQ ID NO: 1306 | LC CDR2 (Kabat) | RASNLES |
| SEQ ID NO: 1307 | LC CDR3 (Kabat) | QQSFDDPFT |
| SEQ ID NO: 1308 | LC CDR1 (Chothia) | SESVDSSGNSF |
| SEQ ID NO: 1306 | LC CDR2 (Chothia) | RASNLES |
| SEQ ID NO: 1307 | LC CDR3 (Chothia) | QQSFDDPFT |
| SEQ ID NO: 1305 | LC CDR1 (Combined) | RASESVDSSGNSFMH |
| SEQ ID NO: 1306 | LC CDR2 (Combined) | RASNLES |
| SEQ ID NO: 1307 | LC CDR3 (Combined) | QQSFDDPFT |
| SEQ ID NO: 3091 | VH | QVQLQQSGPELVKPGASVKISCKASGYAFSSSWMNWV |
| | | KQRPGQGLEWIGRIYPGDGDTKYNGKFKGKATLTADKS |
| | | SSTAYMHLSSLTSVDSAVYFCARRGTGGWYFDVWGAG |
| | | TTVTVSS |
| SEQ ID NO: 3284 | Heavy chain | METDTLLLWVLLLWVPGSTGQVQLQQSGPELVKPGAS |
| | | VKISCKASGYAFSSSWMNWVKQRPGQGLEWIGRIYPGD |
| | | GDTKYNGKFKGKATLTADKSSSTAYMHLSSLTSVDSAV |
| | | YFCARRGTGGWYFDVWGAGTTVTVSSAKTTAPSVYPL |
| | | APVCGDTTGSSVTLGCLVKGYFPEPVTLTWNSGSLSSG |
| | | VHTFPAVLQSDLYTLSSSVTVTSSTWPSQSITCNVAHPA |
| | | SSTKVDKKIEPRGPTIKPCPPCKCPAPNLLGGPSVFIFPPK |
| | | IKDVLMISLSPIVTCVVVDVSEDDPDVQISWFVNNVEVH |
| | | TAQTQTHREDYNSTLRVVSALPIQHQDWMSGKEFKCK |
| | | VNNKDLPAPIERTISKPKGSVRAPQVYVLPPPEEEMTKK |
| | | QVTLTCMVTDFMPEDIYVEWTNNGKTELNYKNTEPVL |
| | | DSDGSYFMYSKLRVEKKNWVERNSYSCSVVHEGLHNH |
| | | HTTKSFSRTPGK |
| SEQ ID NO: 3092 | VL | DIVLTQSPASLAVSLGQRATISCRASESVDSSGNSFMHW |
| | | YQQKPGQPPQLLIYRASNLESGIPARFSGSGSRTDFTLTI |
| | | NPVEADDVATFYCQQSFDDPFTFGSGTKLEIK |
| SEQ ID NO: 3285 | Light chain | METDTLLLWVLLLWVPGSTGDIVLTQSPASLAVSLGQR |
| | | ATISCRASESVDSSGNSFMHWYQQKPGQPPQLLIYRASN |
| | | LESGIPARFSGSGSRTDFTLTINPVEADDVATFYCQQSFD |
| | | DPFTFGSGTKLEIKRADAAPTVSIFPPSSEQLTSGGASVV |
| | | CFLNNFYPKDINVKWKIDGSERQNGVLNSWTDQDSKDS |
| | | TYSMSSTLTLTKDEYERHNSYTCEATHKTSTSPIVKSEN |
| | | RNEC |

Humanized antibody E (E-H antibody)
Variable light chain (VL)

| | | | |
|---|---|---|---|
| SEQ ID NO: 3093 | VL | E-H.1 | DIVLTQSPDSLAVSLGERATINCRASESVDSSGNSFMHWYQQ |
| | | | KPGQPPQLLIYRASNLESGVPDRFSGSGSRTDFTLTISSLQAED |
| | | | VAVYYCQQSFDDPFTFGQGTKLEIK |
| SEQ ID NO: 3094 | VL | E-H.2 | EIVLTQSPATLSLSPGERATLSCRASESVDSSGNSFMHWYQQ |
| | | | KPGQAPQLLIYRASNLESGIPARFSGSGSRTDFTLTISSLEPEDF |
| | | | AVYYCQQSFDDPFTFGQGTKLEIK |
| SEQ ID NO: 3095 | VL | E-H.3 | EIVLTQSPATLSLSPGERATLSCRASESVDSSGNSFMHWYQQ |
| | | | KPGQAPQLLIYRASNLESGIPARFSGSGSRTDFTLTISRLEPED |
| | | | FAVYYCQQSFDDPFTFGQGTKLEIK |
| SEQ ID NO: 3096 | VL | E-H.4 | EIVLTQSPATLSLSPGERATLSCRASESVDSSGNSFMHWYQQ |
| | | | KPGQAPQLLIYRASNLESGIPARFSGSGSRTDFTLTISSLQPED |
| | | | FAVYYCQQSFDDPFTFGQGTKLEIK |
| SEQ ID NO: 3097 | VL | E-H.5 | DIQLTQSPSSLSASVGDRVTITCRASESVDSSGNSFMHWYQQ |
| | | | KPGQAPQLLIYRASNLESGVPSRFSGSGSRTDFTLTISSLQPED |
| | | | VATYYCQQSFDDPFTFGQGTKLEIK |
| SEQ ID NO: 3098 | VL | E-H.6 | EIVLTQSPATLSLSPGERATLSCRASESVDSSGNSFMHWYQQ |
| | | | KPGQAPQLLIYRASNLESGIPARFSGSGPRIDFTLTISSLEPEDF |
| | | | AVYYCQQSEDDPFTFGQGTKLEIK |
| SEQ ID NO: 3099 | VL | E-H.7 | EIVLTQSPATLSLSPGERATLSCRASESVDSSGNSFMHWYQQ |
| | | | KPGQAPQLLIYRASNLESGIPDRFSGSGSRTDFTLTISRLEPED |
| | | | FAVYYCQQSFDDPFTFGQGTKLEIK |
| SEQ ID NO: 3100 | VL | E-H.8 | DIQLTQSPSSLSASVGDRVTITCRASESVDSSGNSFMHWYQQ |
| | | | KPGKVPQLLIYRASNLESGVPSRFSGSGSRTDFTLTISSLQPED |
| | | | VATYYCQQSFDDPFTFGQGTKLEIK |
| SEQ ID NO: 3101 | VL | E-H.9 | DIQLTQSPSSLSASVGDRVTITCRASESVDSSGNSFMHWYQQ |
| | | | KPGKTPQLLIYRASNLESGIPSRFSGSGSRTDFTLTIRSLQPEDF |
| | | | ATYYCQQSFDDPFTFGQGTKLEIK |
| SEQ ID NO: 3102 | VL | E-H.10 | EIVLTQSPGTLSLSPGERATLSCRASESVDSSGNSFMHWYQQ |
| | | | KPGQAPQLLIYRASNLESGIPDRFSGSGSRTDFTLTISRLEPED |
| | | | FAVYYCQQSFDDPFTFGQGTKLEIK |
| SEQ ID NO: 3103 | VL | E-H.11 | EIVLTQSPATLSLSPGERATLSCRASESVDSSGNSFMHWYQQ |
| | | | KPGLAPQLLIYRASNLESGIPDRFSGSGSRTDFTLTISRLEPEDF |
| | | | AVYYCQQSFDDPFTFGQGTKLEIK |
| SEQ ID NO: 3104 | VL | E-H.12 | DIQLTQSPSSLSASVGDRVTITCRASESVDSSGNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3105 | VL | E-H.13 | DIQLTQSPSSVSASVGDRVTITCRASESVDSSQNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3106 | VL | E-H.14 | AIQLTQSPSSLSASVGDRVTITCRASESVDSSGNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3107 | VL | E-H.15 | DIQLTQSPSFLSASVGDRVTITCRASESVDSSGNSFMHWYQQ |
| | | | KPGKAPQLLIYRASNLESGVPSRFSGSGSRTEFTLTISSLQPED |
| | | | FATYYCQQSFDDPFTFGQGTKLEIK |
| SEQ ID NO: 3108 | VL | E-H.16 | DIQLTQSPSSLSASVGDRVTITCRASESVDSSQNSFMHWYQQ |
| | | | KPGKAPQLLIYRASNLESGVPSRFSGSGSRTDFTFTISSLQPEDI |
| | | | ATYYCQQSFDDPFTFGQGTKLEIK |
| SEQ ID NO: 3109 | VL | E-H.17 | EIVLTQSPATLSVSPGERATLSCRASESVDSSGNSFMHWYQQ |
| | | | KPGQAPQLLIYRASNLESGIPARFSGSGSRTEFTLTISILQSEDF |
| | | | AVYYCQQSFDDPFTFGQGTKLEIK |
| SEQ ID NO: 3110 | VL | E-H.18 | EIVLTQSPATLSVSPGERATLSCRASESVDSSGNSFMHWYQQ |
| | | | KPGQAPQLLIYRASNLESGIPARFSGSGSRTEFTLTISSLQSEDF |
| | | | AVYYCQQSFDDPFTFGQGTKLEIK |
| SEQ ID NO: 3111 | VL | E-H.19 | AIRLTQSPFSLSASVGDRVTITCRASESVDSSGNSFMHWYQQ |
| | | | KPAKAPQLFIYRASNLESGVPSRFSGSGSRTDFTLTISSLQPED |
| | | | FATYYCQQSFDDPFTFGQGTKLEIK |
| SEQ ID NO: 3112 | VL | E-H.20 | DIQLTQSPSSLSASVGDRVTITCRASESVDSSQNSFMHWYQQ |
| | | | KPGKAPQSLIYRASNLESGVPSRFSGSGSRTDFTLTISSLQPED |
| | | | FATYYCQQSFDDPFTFGQGTKLEIK |
| SEQ ID NO: 3113 | VL | E-H.21 | DIQLTQSPSSLSASVGDRVTITCRASESVDSSGNSFMHWYQQ |
| | | | KPGKAPQRLIYRASNLESGVPSRFSGSGSRTEFTLTISNLQPED |
| | | | FATYYCQQSFDDPFTFGQGTKLEIK |
| SEQ ID NO: 3114 | VL | E-H.22 | DIQLTQSPSTLSASVGDRVTITCRASESVDSSGNSFMHWYQQ |
| | | | KPGKAPQLLIYRASNLESGVPSRFSGSGSRTEFTLTISSLQPDD |
| | | | FATYYCQQSFDDPFTFGQGTKLEIK |
| SEQ ID NO: 3115 | VL | E-H.23 | EIVLTQSPDFQSVTPKEKVTITCRASESVDSSGNSFMHWYQQ |
| | | | KPDQSPQLLIYRASNLESGVPSRFSGSGSRTDFTLTINSLEAED |
| | | | AATYYCQQSFDDPFTFGQGTKLEIK |
| SEQ ID NO: 3116 | VL | E-H.24 | DIQLTQSPSSLSASVGDRVTITCRASESVDSSGNSEMHWYQQ |
| | | | KPGKAPQSLIYRASNLESGVPSKFSGSGSRTDFTLTISSLQPED |
| | | | FATYYCQQSFDDPFTFGQGTKLEIK |
| SEQ ID NO: 3117 | VL | E-H.25 | DIQLTQSPSSLSASVGDRVTITCRASESVDSSGNSFMHWYQQ |
| | | | KPGKAPQRLIYRASNLESGVPSRFSGSGSRTEFTLTISSLQPED |
| | | | FATYYCQQSFDDPFTFGQGTKLEIK |
| SEQ ID NO: 3118 | VL | E-H.26 | DIVLTQTPLSLSVTPGQPASISCRASESVDSSGNSFMHWYLQK |
| | | | PGQPPQLLIYRASNLESGVPDRFSGSGSRTDFTLKISRVEAED |
| | | | VGVYYCQQSFDDPFTFGQGTKLEIK |
| SEQ ID NO: 3119 | VL | E-H.27 | DIQLTQSPSSLSASVGDRVTITCRASESVDSSGNSFMHWYQQ |
| | | | KPEKAPQSLIYRASNLESGVPSRFSGSGSRTDFTLTISSLQPED |
| | | | FATYYCQQSFDDPFTFGQGTKLEIK |
| SEQ ID NO: 3120 | VL | E-H.28 | EIVLTQSPPTLSLSPGERVTLSCRASESVDSSGNSFMHWYQQK |
| | | | PGQAPQLLIYRASNLESGIPARFSGSGSRTDFTLTISSLQPEDF |
| | | | AVYYCQQSFDDPFTFGQGTKLEIK |
| SEQ ID NO: 3121 | VL | E-H.29 | DIQLTQSPSAMSASVGDRVTITCRASESVDSSGNSFMHWYQQ |
| | | | KPGKVPQRLIYRASNLESGVPSRFSGSGSRTEFTLTISSLQPED |
| | | | FATYYCQQSFDDPFTFGQGTKLEIK |
| SEQ ID NO: 3122 | VL | E-H.30 | DIVLTQSPLSLPVTPGEPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3123 | VL | E-H.31 | DIVLTQTPLSLPVTPGEPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3124 | VL | E-H.32 | DIVLTQTPLSLSVTPGQPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3125 | VL | E-H.33 | EIVLTQSPPTLSLSPGERVTLSCRASESVDSSGNSFMHWYQQK |
| | | | PGQAPQLLIYRASNLESSIPARFSGSGSRTDFTLTISSLQPEDFA |
| | | | VYYCQQSFDDPFTFGQGTKLEIK |
| SEQ ID NO: 3126 | VL | E-H.34 | DIVLTQSPLSLPVTLGQPASISCRASESVDSSGNSFMHWYQQR |
| | | | PGQSPQRLIYRASNLESQVPDRFSGSGSRTDFTLKISRVEAED |
| | | | VGVYYCQQSFDDPFTFGQGTKLEIK |
| SEQ ID NO: 3127 | VL | E-H.35 | DIVLTQTPLSSPVTLGQPASISCRASESVDSSGNSFMHWYQQR |
| | | | PGQPPQLLIYRASNLESGVPDRFSGSGARTDFTLKISRVEAED |
| | | | VGVYYCQQSFDDPFTFGQGTKLEIK |
| SEQ ID NO: 3128 | VL | E-H.36 | DIVLTQSPAFLSVTPGEKVTITCRASESVDSSGNSFMHWYQQ |
| | | | KPDQAPQLLIYRASNLESGVPSRFSGSGSRTDFTFTISSLEAED |
| | | | AATYYCQQSFDDPFTFGQGTKLEIK |
| SEQ ID NO: 3129 | VL | E-H.37 | DIQLIQSPSFLSASVGDRVSIICRASESVDSSGNSFMHWYLQKP |
| | | | GKSPQLFIYRASNLESGVSSRFSGRGSRTDFTLTIISLKPEDFA |
| | | | AYYCQQSFDDPFTFGQGTKLEIK |
| SEQ ID NO: 3130 | VL | E-H.38 | EIVLTQTPLSLSITPGEQASISCRASESVDSSGNSFMHWYLQK |
| | | | ARPVPQLLIYRASNLESGVPDRFSGSGSRTDFTLKISRVEAED |
| | | | FGVYYCQQSFDDPFTFGQGTKLEIK |
| SEQ ID NO: 3131 | VL | E-H.39 | EIVLTQTPLSLSITPGEQASMSCRASESVDSSGNSFMHWYLQK |
| | | | ARPVPQLLIYRASNLESGVPDRFSGSGSRTDFTLKISRVEAED |
| | | | FGVYYCQQSFDDPFTFGQGTKLEIK |
| SEQ ID NO: 3132 | VL | E-H.40 | EITLTQSPAFMSATPGDKVNISCRASESVDSSGNSFMHWYQQ |
| | | | KPGEAPQFIIYRASNLESGIPPRFSGSGYRTDFTLTINNIESEDA |
| | | | AYYYCQQSFDDPFTFGQGTKLEIK |

Variable HEAVY chain (VH)

| | | | |
|---|---|---|---|
| SEQ ID NO: 3133 | VH | E-H.1 | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3134 | VH | E-H.2 | QVQLVQSGAEVKKPGSSVKVSCKASGYAFSSSWMNWVRQA |
| | | | PGQGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3135 | VH | E-H.3 | QVQLVQSGAEVKKPGASVKVSCKASGYAFSSSWMNWVRQA |
| | | | PGKGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3136 | VH | E-H.4 | QVQLVQSGAEVKKPGASVKVSCKASGYAFSSSWMNWVRQA |
| | | | PGQELEWIGRIYPGDGDTKYNGKFKGRATLTADKSISTAYME |
| | | | LSSLRSEDTATYYCARRGTGGWYFDVWGQGTTVTVSS |
| SEQ ID NO: 3137 | VH | E-H.5 | EVQLVQSGAEVKKPGATVKISCKASGYAFSSSWMNWVQQA |
| | | | PGKGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3138 | VH | E-H.6 | QVQLVQSGAEVKKTGSSVKVSCKASGYAFSSSWMNWVRQA |
| | | | PGQALEWIGRIYPGDGDTKYNGKFKGRATLTADKSMSTAYM |
| | | | ELSSLRSEDTAMYYCARRGTGGWYFDVWGQGTTVTVSS |
| SEQ ID NO: 3139 | VH | E-H.7 | QVQLVQSGAEVKKPGASVKVSCKASGYAFSSSWMNWVRQA |
| | | | PGQRLEWIGRIYPGDGDTKYNGKFKGRATLTADKSASTAYM |
| | | | ELSSLRSEDMAVYYCARRGTGGWYFDVWGQGTTVTVSS |
| SEQ ID NO: 3140 | VH | E-H.8 | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELRSLRSDDMAVYYCARRGTGGWYFDVWGQGTTVTVSS |
| SEQ ID NO: 3141 | VH | E-H.9 | QVQLVQSGAEVKKPGASVKVSCKASGYAFSSSWMNWVRQA |
| | | | PGQRLEWIGRIYPGDGDTKYNGKFKGRATLTADKSASTAYM |
| | | | ELSSLRSEDTAVYYCARRGTGGWYFDVWGQGTTVTVSS |
| SEQ ID NO: 3142 | VH | E-H.10 | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELRSLRSDDTAVYYCARRGTGGWYFDVWGQGTTVTVSS |
| SEQ ID NO: 3143 | VH | E-H.11 | QVQLVQSGAEVKKPGASVKVSCKASGYAFSSSWMNWVRQA |
| | | | PGQGLEWIGRIYPGDGDTKYNGKFKGRATLTADKSISTAYM |
| | | | ELSRLRSDDTAVYYCARRGTGGWYFDVWGQGTTVTVSS |
| SEQ ID NO: 3144 | VH | E-H.12 | QVQLVQSGAEVKKPGASVKVSCKASGYAFSSSWMNWVRQA |
| | | | PGQGLEWIGRIYPGDQDTKYNGKFKGRATLTADKSISTAYM |
| | | | ELSRLRSDDTVVYYCARRQTGGWYFDVWGQGTTVTVSS |
| SEQ ID NO: 3145 | VH | E-H.13 | QVQLVQSGAEVKKPGASVKVSCKASGYAFSSSWMNWVRQA |
| | | | PGQGLEWIGRIYPGDGDTKYNGKFKGWATLTADKSISTAYM |
| | | | ELSRLRSDDTAVYYCARRGTGGWYFDVWGQGTTVTVSS |
| SEQ ID NO: 3146 | VH | E-H.14 | QVQLVQSGAEVKKPGASVKVSCKASGYAFSSSWMNWVRQA |
| | | | TGQGLEWIGRIYPGDGDTKYNGKFKGRATLTANKSISTAYM |
| | | | ELSSLRSEDTAVYYCARRGTGGWYFDVWGQGTTVTVSS |
| SEQ ID NO: 3147 | VH | E-H.15 | QVQLVQSGSELKKPGASVKVSCKASGYAFSSSWMNWVRQA |
| | | | PGQGLEWIGRIYPGDGDTKYNGKFKGRAVLSADKSVSTAYL |
| | | | QISSLKAEDTAVYYCARRGTGGWYFDVWGQGTTVTVSS |
| SEQ ID NO: 3148 | VH | E-H.16 | QVQLVQSGPEVKKPGTSVKVSCKASGYAFSSSWMNWVRQA |
| | | | RGQRLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3149 | VH | E-H.17 | EVQLVQSGAEVKKPGESLKISCKASGYAFSSSWMNWVRQMP |
| | | | GKGLEWIGRIYPGDGDTKYNGKFKGQATLSADKSISTAYLQ |
| | | | WSSLKASDTAMYYCARRGTGGWYFDVWGQGTTVTVSS |
| SEQ ID NO: 3150 | VH | E-H.18 | QVQLVQSGSELKKPGASVKVSCKASGYAFSSSWMNWVRQA |
| | | | PGQGLEWIGRIYPGDGDTKYNGKFKGRAVLSADKSVSMAYL |
| | | | QISSLKAEDTAVYYCARRGTGGWYFDVWGQGTTVTVSS |
| SEQ ID NO: 3151 | VH | E-H.19 | QVQLVQSGHEVKQPGASVKVSCKASGYAFSSSWMNWVPQA |
| | | | PGQGLEWIGRIYPGDGDTKYNGKFKGRAVLSADKSASTAYL |
| | | | QISSLKAEDMAMYYCARRGTGGWYFDVWGQGTTVTVSS |
| SEQ ID NO: 3152 | VH | E-H.20 | EVQLVQSGAEVKKPGESLKISCKASGYAFSSSWMNWVRQMP |
| | | | GKGLEWIGRIYPGDGDTKYNGKFKGQATLSADKPISTAYLQ |
| | | | WSSLKASDTAMYYCARRGTGGWYFDVWGQGTTVTVSS |
| SEQ ID NO: 3153 | VH | E-H.21 | EVQLVQSGAEVKKPGESLRISCKASGYAFSSSWMNWVRQMP |
| | | | GKGLEWIGRIYPGDGDTKYNGKFKGQATLSADKSISTAYLQ |
| | | | WSSLKASDTAMYYCARRGTGGWYFDVWGQGTTVTVSS |
| SEQ ID NO: 3154 | VH | E-H.22 | EVQLVQSGAEVKKPGESLRISCKASGYAFSSSWMNWVRQMP |
| | | | GKGLEWIGRIYPGDGDTKYNGKFKGHATLSADKSISTAYLQ |
| | | | WSSLKASDTAMYYCARRGTGGWYFDVWGQGTTVTVSS |
| SEQ ID NO: 3155 | VH | E-H.23 | QVQLVQSGAEVKKTGSSVKVSCKASGYAFSSSWMNWVRQA |
| | | | PRQALEWIGRIYPGDGDTKYNGKFKGRATLTADKSMSTAYM |
| | | | ELSSLRSEDTAMYYCARRGTGGWYFDVWGQGTTVTVSS |
| SEQ ID NO: 3156 | VH | E-H.24 | EVQLVESGGGLVQPGRSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3157 | VH | E-H.25 | EVQLVESGGGLVQPGPSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3158 | VH | E-H.26 | QVQLQESGPGLVKPSQTLSLTCTASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3159 | VH | E-H.27 | QVQLQESGPGLVKPSGTLSLTCAASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3160 | VH | E-H.28 | EVQLVESGGGLVKPGRSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3161 | VH | E-H.29 | EVQLVESGGGLVQPGGSLKLSCAASGYAFSSSWMNWVRQA |
| | | | SGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3162 | VH | E-H.30 | QVQLQESGPGLVKPSQTLSLTCAASGYAFSSSWMNWVRQPP |
| | | | GKGLEWIGRIYPGDQDTKYNGKFKGRATLSADKSKSQASLK |
| | | | LSSVTAADTAVYYCARRGTGQWYFDVWGQGTTVTVSS |
| SEQ ID NO: 3163 | VH | E-H.31 | EVQLVESGGGLVKPGGSLRLSCAASGYAFSSSWMNWVRQA |
| | | | PGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3164 | VH | E-H.32 | EVQLVESGGALVKPGGSLRLSCAASGYAFSSSWMNWVRQA |
| | | | PGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3165 | VH | E-H.33 | QVQLQESGPGLVKPSQTLSLTCAAYGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3166 | VH | E-H.34 | QVQLQESGSGLVKPSQTLSLTCAASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3167 | VH | E-H.35 | EVQLVESGGGLVQPGGSLRLSCAASGYAFSSSWMNWVRQA |
| | | | PGKGLEWIGRIYPGDGDTKYNGKFKGRATLSADKSKSSAYL |
| | | | QMNSLKTEDTAVYYCARRGTGGWYFDVWGQGTTVTVSS |
| SEQ ID NO: 3168 | VH | E-H.36 | QVQLQESGPGLVKPSDTLSLTCTASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3169 | VH | E-H.37 | QVQLQESGPGLVKPSQTLSLTCTASGYAFSSSWMNWVRQHP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3170 | VH | E-H.38 | QVQLQESGPGLVKPSQTLSLTCTASGYAFSSSWMNWVRQHP |
| | | | GKGLEWIGRIYPGDGDTKYNGKFKGLATLSADKSKSQASLK |
| | | | LSSVTAADTAVYYCARRGTGGWYFDVWGQGTTVTVSS |
| SEQ ID NO: 3171 | VH | E-H.39 | QVQLVESGGGVVQPGRSLRLSCAASGYAFSSSWMNWVRQA |
| | | | PGKGLEWIGRIYPGDGDTKYNGKFKGRATLSADKSKSTAYL |
| | | | QMSSLRAEDTAVYYCARRGTGGWYFDVWGQGTTVTVSS |
| SEQ ID NO: 3172 | VH | E-H.40 | QVQLVESGGGLVKPGGSLRLSCAASGYAFSSSWMNWVRQA |
| | | | PGKGLEWIGRIYPGDGDTKYNGKFKGRATLSADKAKSSAYL |
| | | | QMNSLRAEDTAVYYCARRGTGGWYFDVWGQGTTVTVSS |
| SEQ ID NO: 3173 | VH | E-H.41 | QVQLVESGGGLVQPGGSLRLSCSASGYAFSSSWMNWVRQAP |
| | | | GKGLEWIGRIYPGDGDTKYNGKFKGRATLSADKSKSTAYLQ |
| | | | MNSLRAEDTAVYYCARRGTGGWYFDVWGQGTTVTVSS |
| SEQ ID NO: 3174 | VH | E-H.42 | QVQLLESGGGLVKPGGSLRLSCAASGYAFSSSWMNWVRQA |
| | | | PGKGLEWIGRIYPGDGDTKYNGKFKGRATLSADKAKSSAYL |
| | | | QMNSLRAEDTAVYYCARRGTGGWYFDVWGQGTTVTVSS |
| SEQ ID NO: 3175 | VH | E-H.43 | EVQLVESGGGLVQPGGSLRLSCSASGYAFSSSWMNWVRQAP |
| | | | GKGLEWIGRIYPGDGDTKYNGKFKGRATLSADKSKSTAYLQ |
| | | | MSSLRAEDTAVYYCARRGTGGWYFDVWGQGTTVTVSS |
| SEQ ID NO: 3176 | VH | E-H.44 | QVQLQESGPGLVKPSDTLSLTCAASGYAFSSSWMNWVRQPP |
| | | | GKGLEWIGRIYPGDGDTKYNGKFKGRATISADKSKSQASLK |
| | | | LSSVTAVDTAVYYCARRGTGGWYFDVWGQGTTVTVSS |
| SEQ ID NO: 3177 | VH | E-H.45 | QVQLQESGPGLVKPSQTLSLTCAASGYAFSSSWMNWVRQPP |
| | | | GKGLEWIGRIYPGDGDTKYNGKFKGRATLSADKSKSQASLK |
| | | | LSSVTAVDTAVYYCARRGTGGWYFDVWGQGTTVTVSS |
| SEQ ID NO: 3178 | VH | E-H.46 | EVQLVESGGGLVQPGGSLRLSCSASGYAFSSSWMNWVRQAP |
| | | | GKGLEWIGRIYPGDGDTKYNGKFKGRATLSADKSKSTAYVQ |
| | | | MSSLRAEDTAVYYCARRGTGGWYFDVWGQGTTVTVSS |
| SEQ ID NO: 3179 | VH | E-H.47 | QVQLVDSGGGVVQPGRSLRLSCAASGYAFSSSWMNWVRQA |
| | | | PGKGLEWIGRIYPGDGDTKYNGKFKGRATLSADKSKSTAYL |
| | | | QMNSLRAEDTAVYYCARRGTGGWYFDVWGQGTTVTVSS |
| SEQ ID NO: 3180 | VH | E-H.48 | QVQLVESGGGVVQPGRSLRLSCAASGYAFSSSWMNWVRQA |
| | | | PGKGLEWIGRIYPGDQDTKYNGKFKGRATLSADKSKSTAYL |
| | | | QMNSLRAEGTAVYYCARRGTGGWYFDVWGQGTTVTVSS |
| SEQ ID NO: 3181 | VH | E-H.49 | QVQLVESGGGVVQPGRSLRLSCAASGYAFSSSWMNWVRQA |
| | | | PGKGLEWIGRIYPGDGDTKYNGKFKGRATLSADKSKSTAYL |
| | | | QMNSLRAEDTAVYYCARRGTGGWYFDVWGQGTTVTVSS |
| SEQ ID NO: 3182 | VH | E-H.50 | EVQLVESGGGLVQPGGSLRLSCAASGYAFSSSWMNWVRQA |
| | | | PGKGLEWIGRIYPQDGDTKYNGKFKGRATLSADKSKSTAYL |
| | | | QMNSLRAEDTAVYYCARRQTGGWYFDVWGQGTTVTVSS |

TABLE 12
Amino acid sequences for anti TCRβ V10 antibodies. Amino acid and nucleotide sequences for
murine and humanized antibody molecules which bind to TCRBV 10 (c.g., TCRBV 10-1, TCRBV 10-2
or TCRBV 10-3). The amino acid the heavy and light chain CDRs, and the amino acid and
nucleotide sequences of the heavy and light chain variable regions, and the heavy and light
chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined)) | LISSGGSYTYYTDSVKG |
| SEQ ID NO: 1290 | HC CDR3 (Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSYGMSWVR |
| | | QTPDKRLEWVALISSGGSYTYYTDSVKGRFTISRDNAKN |
| | | TLYLQMSSLKSEDTAIYYCSRHGGNFFDYWGQGTTLTVS |
| | | S |
| SEQ ID NO: 3184 | VL | QIVLTQSPSIMSASPGEKVTMTCSVSSSVSYMHWYQQKS |
| | | GTSPKRWIYDTSKLASGVPARFSGSGSGTSYSLTISSMEA |
| | | EDAATYYCQQWSSNPQYTFGGGTKLEIK |

Hamanized antibody D (D-H antibody)
Variable light chain (VL)

| | | | |
|---|---|---|---|
| SEQ ID NO: 3185 | VL | D-H.1 | DIVLTQSPAFLSVTPGEKVTITCSVSSSVSYMHWYQQKPDQAPK |
| | | | LLIYDTSKLASGVPSRFSGSGSGTDYTFTISSLEAEDAATYYCQQ |
| | | | WSSNPQYTFGQGTKLEIK |
| SEQ ID NO: 3186 | VL | D-H.2 | AIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYYTFGQGTKLEIK |
| SEQ ID NO: 3187 | VL | D-H.3 | DIQLTQSPSFLSASVGDRVTITCSVSSSVSYMHWYQQKPGKAPK |
| | | | LLIYDTSKLASGVPSRFSGSGSGTEYTLTISSLQPEDFATYYCQQ |
| | | | WSSNPQYTFGQGTKLEIK |
| SEQ ID NO: 3188 | VL | D-H.4 | DIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3189 | VL | D-H.5 | DIQLTQSPSSVSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3190 | VL | D-H.6 | DIQLTQSPSSLSASVGDRVTITCSVSSSVSYMHWYQQKPGKVPK |
| | | | LLIYDTSKLASGVPSRFSGSGSGTDYTLTISSLQPEDVATYYCQQ |
| | | | WSSNPQYTFGQGTKLEIK |
| SEQ ID NO: 3191 | VL | D-H.7 | DIQLTQSPSSLSASVGDRVTITCSVSSSVSYMHWYQQKPGQAPK |
| | | | LLIYDTSKLASGVPSRFSGSGSGTDYTLTISSLQPEDVATYYCQQ |
| | | | WSSNPQYTFGQGTKLEIK |
| SEQ ID NO: 3192 | VL | D-H.8 | EIVLTQSPDFQSVTPKEKVTITCSVSSSVSYMHWYQQKPDQSPKL |
| | | | LIYDTSKLASGVPSRFSGSGSGTDYTLTINSLEAEDAATYYCQQ |
| | | | WSSNPQYTFGQGTKLEIK |
| SEQ ID NO: 3193 | VL | D-H.9 | AIRLTQSPFSLSASVGDRVTITCSVSSSVSYMHWYQQKPAKAPK |
| | | | LFIYDTSKLASGVPSRFSGSGSGTDYTLTISSLQPEDFATYYCQQ |
| | | | WSSNPQYTFGQGTKLEIK |
| SEQ ID NO: 3194 | VL | D- | DIQLTQSPSSLSASVGDRVTITCSVSSSVSYMHWYQQKPGKAPK |
| | | H.10 | LLIYDTSKLASGVPSRFSGSGSGTDYTFTISSLQPEDIATYYCQQ |
| | | | WSSNPQYTFGQGTKLEIK |
| SEQ ID NO: 3195 | VL | D- | EIVLTQSPATLSLSPGERATLSCSVSSSVSYMHWYQQKPGQAPK |
| | | H.11 | LLIYDTSKLASGIPARFSGSGSGTDYTLTISSLEPEDFAVYYCQQ |
| | | | WSSNPQYTFGQGTKLEIK |
| SEQ ID NO: 3196 | VL | D- | DIQLTQSPSTLSASVGDRVTITCSVSSSVSYMHWYQQKPGKAPK |
| | | H.12 | LLIYDTSKLASGVPSRFSGSGSGTEYTLTISSLQPDDFATYYCQQ |
| | | | WSSNPQYTFGQGTKLEIK |
| SEQ ID NO: 3197 | VL | D- | DIQLTQSPSSLSASVGDRVTITCSVSSSVSYMHWYQQKPGKTPK |
| | | H.13 | LLIYDTSKLASGIPSRFSGSGSGTDYTLTIRSLQPEDFATYYCQQ |
| | | | WSSNPQYTFGQGTKLEIK |
| SEQ ID NO: 3198 | VL | D- | EIVLTQSPPTLSLSPGERVTLSCSVSSSVSYMHWYQQKPGQAPKL |
| | | H.14 | LIYDTSKLASGIPARFSGSGSGTDYTLTISSLQPEDFAVYYCQQW |
| | | | SSNPQYTFGQGTKLEIK |
| SEQ ID NO: 3199 | VL | D- | DIQLTQSPSSLSASVGDRVTITCSVSSSVSYMHWYQQKPGKAPK |
| | | H.15 | RLIYDTSKLASGVPSRFSGSGSGTEYTLTISSLQPEDFATYYCQQ |
| | | | WSSNPQYTFGQGTKLEIK |
| SEQ ID NO: 3200 | VL | D- | EIVLTQSPATLSLSPGERATLSCSVSSSVSYMHWYQQKPGQAPK |
| | | H.16 | LLIYDTSKLASGIPARFSGSGPGTDYTLTISSLEPEDFAVYYCQQ |
| | | | WSSNPQYTFGQGTKLEIK |
| SEQ ID NO: 3201 | VL | D- | EIVLTQSPATLSLSPGERATLSCSVSSSVSYMHWYQQKPGQAPK |
| | | H.17 | LLIYDTSKLASGIPARFSGSGSGTDYTLTISRLEPEDFAVYYCQQ |
| | | | WSSNPQYTFGQGTKLEIK |
| SEQ ID NO: 3202 | VL | D- | EIVLTQSPATLSLSPGERATLSCSVSSSVSYMHWYQQKPGQAPK |
| | | H.18 | LLIYDTSKLASGIPARFSGSGSGTDYTLTISSLQPEDFAVYYCQQ |
| | | | WSSNPQYTFGQGTKLEIK |
| SEQ ID NO: 3203 | VL | D- | EIVLTQSPATLSVSPGERATLSCSVSSSVSYMHWYQQKPGQAPK |
| | | H.19 | LLIYDTSKLASGIPARFSGSGSGTEYTLTISSLQSEDFAVYYCQQ |
| | | | WSSNPQYTFGQGTKLEIK |
| SEQ ID NO: 3204 | VL | D- | EIVLTQSPATLSVSPGERATLSCSVSSSVSYMHWYQQKPGQAPK |
| | | H.20 | LLIYDTSKLASGIPARFSGSGSGTEYTLTISILQSEDFAVYYCQQW |
| | | | SSNPQYTFGQGTKLEIK |
| SEQ ID NO: 3205 | VL | D- | EIVLTQSPPTLSLSPGERVTLSCSVSSSVSYMHWYQQKPGQAPKL |
| | | H.21 | LIYDTSKLASSIPARFSGSQSGTDYTLTISSLQPEDFAVYYCQQWS |
| | | | SNPQYTFGQGTKLEIK |
| SEQ ID NO: 3206 | VL | D- | DIQLTQSPSSLSASVGDRVTITCSVSSSVSYMHWYQQKPGKAPK |
| | | H.22 | SLIYDTSKLASGVPSRFSGSGSGTDYTLTISSLQPEDFATYYCQQ |
| | | | WSSNPQYTFGQGTKLEIK |
| SEQ ID NO: 3207 | VL | D- | DIQLTQSPSSLSASVGDRVTITCSVSSSVSYMHWYQQKPGKAPK |
| | | H.23 | RLIYDTSKLASGVPSRFSGSGSGTEYTLTISNLQPEDFATYYCQQ |
| | | | WSSNPQYTFGQGTKLEIK |
| SEQ ID NO: 3208 | VL | D- | DIQLTQSPSAMSASVGDRVTITCSVSSSVSYMHWYQQKPGKVPK |
| | | H.24 | RLIYDTSKLASGVPSRFSGSGSGTEYTLTISSLQPEDFATYYCQQ |
| | | | WSSNPQYTFGQGTKLEIK |
| SEQ ID NO: 3209 | VL | D. | EIVLTQSPATLSLSPGERATLSCSVSSSVSYMHWYQQKPGQAPK |
| | | H.25 | LLIYDTSKLASGIPDRFSGSGSGTDYTLTISRLEPEDFAVYYCQQ |
| | | | WSSNPQYTFGQGTKLEIK |
| SEQ ID NO: 3210 | VL | D- | EIVLTQSPATLSLSPGERATLSCSVSSSVSYMHWYQQKPGLAPKL |
| | | H.26 | LIYDTSKLASGIPDRESGSGSGTDYTLTISRLEPEDFAVYYCQQW |
| | | | SSNPQYTFGQGTKLEIK |
| SEQ ID NO: 3211 | VL | D- | EIVLTQSPGTLSLSPGERATLSCSVSSSVSYMHWYQQKPGQAPK |
| | | H.27 | LLIYDTSKLASGIPDRFSGSGSGTDYTLTISRLEPEDFAVYYCQQ |
| | | | WSSNPQYTFGQGTKLEIK |
| SEQ ID NO: 3212 | VL | D- | DIQLTQSPSSLSASVGDRVTITCSVSSSVSYMHWYQQKPGKAPK |
| | | H.28 | SLIYDTSKLASGVPSKFSGSGSGTDYTLTISSLQPEDFATYYCQQ |
| | | | WSSNPQYTFGQGTKLEIK |
| SEQ ID NO: 3213 | VL | D- | DIQLTQSPSSLSASVGDRVTITCSVSSSVSYMHWYQQKPEKAPKS |
| | | H.29 | LIYDTSKLASGVPSRFSGSGSGTDYTLTISSLQPEDFATYYCQQW |
| | | | SSNPQYTFGQGTKLEIK |
| SEQ ID NO: 3214 | VL | D- | DIVLTQSPDSLAVSLGERATINCSVSSSVSYMHWYQQKPGQPPK |
| | | H.30 | LLIYDTSKLASGVPDRFSGSGSGTDYTLTISSLQAEDVAVYYCQQ |
| | | | WSSNPQYTFGQGTKLEIK |
| SEQ ID NO: 3215 | VL | D- | EIVLTQTPLSLSITPGEQASMSCSVSSSVSYMHWYLQKARPVPKL |
| | | H.31 | LIYDTSKLASGVPDRFSGSGSGTDYTLKISRVEAEDFGVYYCQQ |
| | | | WSSNPQYTFGQGTKLEIK |
| SEQ ID NO: 3216 | VL | D- | EIVLTQTPLSLSITPGEQASISCSVSSSVSYMHWYLQKARPVPKLL |
| | | H.32 | IYDTSKLASGVPDRFSGSGSGTDYTLKISRVEAEDFQVYYCQQW |
| | | | SSNPQYTFGQGTKLEIK |
| SEQ ID NO: 3217 | VL | D- | DIVLTQSPLSLPVTPGEPASISCSVSSSVSYMHWYLQKPGQSPKL |
| | | H.33 | LIYDTSKLASGVPDRFSGSGSGTDYTLKISRVEAEDVGVYYCQQ |
| | | | WSSNPQYTFGQGTKLEIK |
| SEQ ID NO: 3218 | VL | D- | DIVLTQSPLSLPVTLGQPASISCSVSSSVSYMHWYQQRPGQSPKR |
| | | H.34 | LIYDTSKLASGVPDRFSGSGSGTDYTLKISRVEAEDVGVYYCQQ |
| | | | WSSNPQYTFGQGTKLEIK |
| SEQ ID NO: 3219 | VL | D- | DIVLTQTPLSLPVTPGEPASISCSVSSSVSYMHWYLQKPGQSPKL |
| | | H.35 | LIYDTSKLASGVPDRFSGSGSGTDYTLKISRVEAEDVGVYYCQQ |
| | | | WSSNPQYTFGQGTKLEIK |
| SEQ ID NO: 3220 | VL | D- | DIVLTQTPLSLSVTPGQPASISCSVSSSVSYMHWYLQKPGQSPKL |
| | | H.36 | LIYDTSKLASGVPDRFSGSGSGTDYTLKISRVEAEDVGVYYCQQ |
| | | | WSSNPQYTFGQGTKLEIK |
| SEQ ID NO: 3221 | VL | D- | DIVLTQTPLSLSVTPGQPASISCSVSSSVSYMHWYLQKPGQPPKL |
| | | H.37 | LIYDTSKLASGVPDRFSGSGSGTDYTLKISRVEAEDVGVYYCQQ |
| | | | WSSNPQYTFGQGTKLEIK |
| SEQ ID NO: 3222 | VL | D. | DIQLIQSPSFLSASVGDRVSIICSVSSSVSYMHWYLQKPGKSPKLF |
| | | H.38 | IYDTSKLASGVSSRFSGRGSGTDYTLTIISLKPEDFAAYYCQQWS |
| | | | SNPQYTFGQGTKLEIK |
| SEQ ID NO: 3223 | VL | D- | DIVLTQTPLSSPVTLGQPASISCSVSSSVSYMHWYQQRPGQPPKL |
| | | H.39 | LIYDTSKLASGVPDRFSGSGAGTDYTLKISRVEAEDVGVYYCQQ |
| | | | WSSNPQYTFGQGTKLEIK |
| SEQ ID NO: 3224 | VL | D- | EITLTQSPAFMSATPGDKVNISCSVSSSVSYMHWYQQKPGEAPK |
| | | H.40 | FIIYDTSKLASGIPPRFSGSGYGTDYTLTINNIESEDAAYYYCQQW |
| | | | SSNPQYTFGQGTKLEIK |

Variable HEAVY chain (VH)

| | | | |
|---|---|---|---|
| SEQ ID NO: 3225 | VH | D-H.1 | EVQLVESGGGLVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3226 | VH | D-H.2 | EVQLVESGGALVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3227 | VH | D-H.3 | EVQLVESGGGLVQPGGSLRLSCAVSGFTFRSYGMSWVRQAPGK |
| | | | GLEWVALISSGGSYTYYTDSVKGRFTISRDNAKNTLYLQMNSLR |
| | | | AEDTAVYYCSRHGGNFFDYWGQGTTVTVSS |
| SEQ ID NO: 3228 | VH | D-H.4 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3229 | VH | D-H.5 | EVQLVESGGGLVQPGGSLRLSCAVSGFTFRSYGMSWVRQAPGK |
| | | | GLEWVALISSGGSYTYYTDSVKGRFTISRDNSKNSLYLQMNSLK |
| | | | TEDTAVYYCSRHGGNFFDYWGQGTTVTVSS |
| SEQ ID NO: 3230 | VH | D-H.6 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDMAVYYCSRHGGNFFDYWGQGTTVTVSS |
| SEQ ID NO: 3231 | VH | D-H.7 | EVQLVESGGGLVQPGGSLRLSCAVSGFTFRSYGMSWVRQAPGK |
| | | | GLEWVALISSGGSYTYYTDSVKGQFTISRDNAKNTLYLQMNSLR |
| | | | AEDMAVYYCSRHGGNFFDYWGQGTTVTVSS |
| SEQ ID NO: 3232 | VH | D-H.8 | EVQLVESGGGLVKPGRSLRLSCTVSGFTFRSYGMSWVRQAPGK |
| | | | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3233 | VH | D-H.9 | EVQLVESGGGLVKPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3234 | VH | D- | EVQLVESGGGLVQPGGSLKLSCAVSGFTFRSYGMSWVRQASGK |
| | | H.10 | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3235 | VH | D- | QVQLVESGGGVVQPGGSLRLSCAVSGFTFRSYGMSWVRQAPGK |
| | | H.11 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3236 | VH | D- | QVQLVESGGGVVQPGRSLRLSCAVSGFTFRSYGMSWVRQAPGK |
| | | H.12 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3237 | VH | D- | EVQLVESGGGLVQPGGSLRLSCPVSGFTFRSYGMSWVRQAPGK |
| | | H.13 | GLEWVALISSGGSYTYYTDSVKGRFTISRDNANNSLYLQMNSLR |
| | | | AEDTAVYYCSRHGGNFFDYWGQGTTVTVSS |
| SEQ ID NO: 3238 | VH | D- | EVQLVESGGGLVQPGRSLRLSCTVSGFTFRSYGMSWVRQAPGK |
| | | H.14 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3239 | VH | D- | EVQLVESGGGLVQPGPSLRLSCTVSGFTFRSYGMSWVRQAPGK |
| | | H.15 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3240 | VH | D- | EVQLVESGGGLVQPGGSLRLSCAVSGFTFRSYGMSWVRQAPGK |
| | | H.16 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3241 | VH | D- | EVQLVESGQGLVQPGGSLRLSCAVSGFTFRSYGMSWVRQAPGK |
| | | H.17 | GLEWVALISSGGSYTYYTDSVKGRFTISRDNAKNSLYLQMNSLR |
| | | | DEDTAVYYCSRHGGNFFDYWGQGTTVTVSS |
| SEQ ID NO: 3242 | VH | D- | QVQLVESGGGLVKPGGSLRLSCAVSGFTFRSYGMSWVRQAPGK |
| | | H.18 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3243 | VH | D- | QVQLVESGGGVVQPGRSLRLSCAVSGFTFRSYGMSWVRQAPGK |
| | | H.19 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3244 | VH | D- | EVQLLESGGGLVQPGGSLRLSCAVSGFTFRSYGMSWVRQAPGK |
| | | H.20 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3245 | VH | D- | EVQLVESGGGLVQPGGSLRLSCAVSGFTFRSYGMSWVRQAPGK |
| | | H.21 | GLEWVALISSGGSYTYYTDSVKGRFTISRHNSKNTLYLQMNSLR |
| | | | AEDTAVYYCSRHGGNFFDYWGQGTTVTVSS |
| SEQ ID NO: 3246 | VH | D- | EVQLVESGGGLIQPGGSLRLSCAVSGFTFRSYGMSWVRQPPGKG |
| | | H.22 | LEWVALISSGGSYTYYTDSVKGRFTISRDNSKNTLYLQMNSLRA |
| | | | EDTAVYYCSRHGGNFFDYWGQGTTVTVSS |
| SEQ ID NO: 3247 | VH | D- | EVQLVESGGGLIQPGGSLRLSCAVSGFTFRSYGMSWVRQAPGK |
| | | H.23 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3248 | VH | D- | EVQLVESGGGLVQPGRSLRLSCAVSGFTFRSYGMSWVRQAPGK |
| | | H.24 | GLEWVALISSGGSYTYYTDSVKGRFTISRDNAKNSLYLQMNSLR |
| | | | AEDTALYYCSRHGGNFFDYWGQGTTVTVSS |
| SEQ ID NO: 3249 | VH | D- | QVQLVESGGGVVQPGRSLRLSCAVSGFTFRSYGMSWVRQAPGK |
| | | H.25 | GLEWVALISSGGSYTYYTDSVKGRFTISRDNSKNRLYLQMNSLR |
| | | | AEDTAVYYCSRHGGNFFDYWGQGTTVTVSS |
| SEQ ID NO: 3250 | VH | D- | QVQLVESGGGVVQPGRSLRLSCAVSGFTFRSYGMSWVRQAPGK |
| | | H.26 | GLEWVALISSGGSYTYYTDSVKGRFTISRDNSKNTLYLQMNSLR |
| | | | AEGTAVYYCSRHGGNFFDYWGQGTTVTVSS |
| SEQ ID NO: 3251 | VH | D- | QVQLVESGGGVVQPGRSLRLSCAVSGFTFRSYGMSWVRQAPGK |
| | | H.27 | GLEWVALISSGGSYTYYTDSVKGRFAISRDNSKNTLYLQMNSLR |
| | | | AEDTAVYYCSRHGGNFFDYWGQGTTVTVSS |
| SEQ ID NO: 3252 | VH | D- | QVQLVDSGGGVVQPGRSLRLSCAVSGFTFRSYGMSWVRQAPG |
| | | H.28 | KGLEWVALISSGGSYTYYTDSVKGRFTISRDNSKNTLYLQMNSL |
| | | | RAEDTAVYYCSRHGGNFFDYWGQGTTVTVSS |
| SEQ ID NO: 3253 | VH | D- | EVQLVESGGGVVRPGGSLRLSCAVSGFTFRSYGMSWVRQAPGK |
| | | H.29 | GLEWVALISSGGSYTYYTDSVKGRFTISRDNAKNSLYLQMNSLR |
| | | | AEDTALYHCSRHGGNFFDYWGQGTTVTVSS |
| SEQ ID NO: 3254 | VH | D- | EVQLVESGGVVVQPGGSLRLSCAVSGFTFRSYGMSWVRQAPGK |
| | | H.30 | GLEWVALISSGGSYTYYTDSVKGRFTISRDNSKNSLYLQMNSLR |
| | | | AEDTALYYCSRHGGNFFDYWGQGTTVTVSS |
| SEQ ID NO: 3255 | VH | D- | EVQLVESGGGVVQPGGSLRLSCAVSGFTFRSYGMSWVRQAPGK |
| | | H.31 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3256 | VH | D- | EVQLVESGGVVVQPGGSLRLSCAVSGFTFRSYGMSWVRQAPQK |
| | | H.32 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3257 | VH | D- | EVQLVETGGGLIQPGGSLRLSCAVSGFTFRSYGMSWVRQAPGK |
| | | H.33 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3258 | VH | D- | EVQLVESGGGLVQPGGSLRLSCAVSGFTFRSYGMSWVRQATGK |
| | | H.34 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3259 | VH | D- | EVQLVESRGVLVQPGGSLRLSCAVSGFTFRSYGMSWVRQAPGK |
| | | H.35 | GLEWVALISSGGSYTYYTDSVKGRFTISRDNSKNTLHLQMNSLR |
| | | | AEDTAVYYCSRHGGNFFDYWGQGTTVTVSS |
| SEQ ID NO: 3260 | VH | D- | EVQLVESGGGLVQPGRSLRLSCAVSGFTFRSYGMSWVRQAPGK |
| | | H.36 | GLEWVALISSGGSYTYYTDSVKGRFTISRDNAKNSLYLQMNSLR |
| | | | AEDMALYYCSRHGGNFFDYWGQGTTVTVSS |
| SEQ ID NO: 3261 | VH | D- | QVQLVESGGGLVQPGGSLRLSCSVSGFTFRSYGMSWVRQAPGK |
| | | H.37 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3262 | VH | D- | EVQLVESGGGLVQPGGSLRLSCSVSGFTFRSYGMSWVRQAPGK |
| | | H.38 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3263 | VH | D. | QVQLVESGGGVVQPGRSLRLSCAVSGFTFRSYGMSWVRQAPGK |
| | | H.39 | GLEWVALISSGGSYTYYTDSVKGRFTISRDNSTNTLFLQMNSLR |
| | | | AEDTAVYYCSRHGGNFFDYWGQGTTVTVSS |
| SEQ ID NO: 3264 | VH | D- | QVQLLESGGGLVKPGGSLRLSCAVSGFTFRSYGMSWVRQAPGK |
| | | H.40 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3265 | VH | D- | EVQLVESGEGLVQPGGSLRLSCAVSGFTFRSYGMSWVRQAPGK |
| | | H.41 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3266 | VH | D- | EVQLVESGGGLVQPGGSLRLSCAVSGFTFRSYGMSWVRQAPGK |
| | | H.42 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3267 | VH | D. | EVQLVESGGGLVQPGGSLRLSCSVSGFTFRSYGMSWVRQAPGK |
| | | H.43 | GLEWVALISSGGSYTYYTDSVKGRFTISRDNSKNTLYVQMSSLR |
| | | | AEDTAVYYCSRHGGNFFDYWGQGTTVTVSS |
| SEQ ID NO: 3268 | VH | D- | EVQLVESGGGLVQPGGSLRLSCAVSGFTFRSYGMSWVRQAPGK |
| | | H.44 | GLEWVALISSGGSYTYYTDSVKGRFIISRDNSRNSLYLQKNRRR |
| | | | AEDMAVYYCSRHGGNFFDYWGQGTTVTVSS |
| SEQ ID NO: 3269 | VH | D- | EVQLVESGGGLVQPGGSLRLSCAVSGFTFRSYGMSWVHQAPGK |
| | | H.45 | GLEWVALISSGGSYTYYTDSVKGRFIISRDNSRNTLYLQTNSLRA |
| | | | EDTAVYYCSRHGGNFFDYWGQGTTVTVSS |
| SEQ ID NO: 3270 | VH | D- | EVHLVESGGGLVQPGGALRLSCAVSGFTFRSYGMSWVRQATGK |
| | | H.46 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3271 | VH | D- | EVQLVESGGGLVQPRGSLRLSCAVSGFTFRSYGMSWVRQAPGK |
| | | H.47 | GLEWVALISSGGSYTYYTDSVKGRFTISRDNSKNTLYLQMNNLR |
| | | | AEGTAVYYCSRHGGNFFDYWGQGTTVTVSS |
| SEQ ID NO: 3272 | VH | D- | EVQLVESGGGLVQPRGSLRLSCAVSGFTFRSYGMSWVRQAPGK |
| | | H.48 | GLEWVALISSGGSYTYYTDSVKGRFTISRDNSKNTLYLQMNNLR |
| | | | AEGTAAYYCSRHGGNFFDYWGQGTTVTVSS |
| SEQ ID NO: 3273 | VH | D- | QVQLVQSGAEVKKPGASVKVSCKVSGFTFRSYGMSWVRQAPG |
| | | H.49 | KGLEWVALISSGGSYTYYTDSVKGRFTITRDNSTNTLYMELSSL |
| | | | RSEDTAVYYCSRHGGNFFDYWGQGTTVTVSS |
| SEQ ID NO: 3274 | VH | D- | QVQLVQSGSELKKPGASVKVSCKVSGFTFRSYGMSWVRQAPGQ |
| | | H.50 | GLEWVALISSGGSYTYYTDSVKGRFVISRDNSVNTLYLQISSLKA |
| | | | EDTAVYYCSRHGGNFFDYWGQGTTVTVSS |

TABLE 13
Amino acid sequences for additional anti-TCRβ V antibodies. Amino acid and nucleotide
sequences for murine and humanized antibody molecules which bind to various TCRVB families are
disclosed. The amino acid the heavy and light chain CDRs, and the amino acid and nucleotide
sequences of the heavy and light chain variable regions, and the heavy and light chains are
shown. Antibodies disclosed in the table include, MPB2D5, CAS1.1.3, IMMU222, REA 1062, and
JOVI-3. MPB2D5 binds human TCRβV 20-1 (TCRβV2 per old nomenclature). CAS1.1.3 binds human
TCRβV 27 (TCRβV 14 per old nomenclature). IMMU 222 binds human TCRβV 6-5, TCRβV 6-6, or TCRβV
6-9 (TCRβV13.1 per old nomenclature) REA1062 binds human TCRβV 5-1). JOVI-3 binds human
TCRβV 28 (TCRβV3.1 per old nomenclature). IMMU546 binds human TCRβV 2.
MPB2D5 (murine), also referred to here as BJ1188, BJ1190 and REA654; or Antibody G
Binds to human TCRVβ 20-1

| | | |
|---|---|---|
| SEQ ID NO: 1102 | HC CDR1 (Kabat) | SAYMH |
| SEQ ID NO: 1103 | HC CDR2 (Kabat) | RIDPATGKTKYAPKFQA |
| SEQ ID NO: 1104 | HC CDR3 (Kabat) | SLNWDYGLDY |
| SEQ ID NO: 1105 | HC CDR1 (Chothia) | GFNIKSA |
| SEQ ID NO: 1106 | HC CDR2 (Chothia) | DPATGK |
| SEQ ID NO: 1104 | HC CDR3 (Chothia) | SLNWDYGLDY |
| SEQ ID NO: 3640 | HC CDR1 (Combined) | GFNIKSAYMH |
| SEQ ID NO: 1103 | HC CDR2 (Combined) | RIDPATGKTKYAPKFQA |
| SEQ ID NO: 1104 | HC CDR3 (Combined) | SLNWDYGLDY |
| SEQ ID NO: 1107 | LC CDR1 (Kabat) | RASKSVSILGTHLIH |
| SEQ ID NO: 1108 | LC CDR2 (Kabat) | AASNLES |
| SEQ ID NO: 1109 | LC CDR3 (Kabat) | QQSIEDPWT |
| SEQ ID NO: 1110 | LC CDR1 (Chothia) | SKSVSILGTHL |
| SEQ ID NO: 1108 | LC CDR2 (Chothia) | AASNLES |
| SEQ ID NO: 1109 | LC CDR3 (Chothia) | QQSIEDPWT |
| SEQ ID NO: 1107 | LC CDR1 (Combined) | RASKSVSILGTHLIH |
| SEQ ID NO: 1108 | LC CDR2 (Combined) | AASNLES |
| SEQ ID NO: 1109 | LC CDR3 (Combined) | QQSIEDPWT |
| SEQ ID NO: 1111 | VL | DIVLTQSPASLAVSLGQRATISCRASKSVSILGTHLIHWYQQKP |
| | | GQPPKLLIYAASNLESGVPARFSGSGSETVFTLNIHPVEEEDAA |
| | | TYFCQQSIEDPWTFGGGTKLGIK |
| SEQ ID NO: 1112 | VH | EVQLQQSVADLVRPGASLKLSCTASGFNIKSÅYMHWVIQRPD |
| | | QGPECLGRIDPATGKTKYAPKFQAKATITADTSSNTAYLQLSS |
| | | LTSEDTAIYYCTRSLNWDYGLDYWGQGTSVTVSS |

VH for MPB2D5 (humanized) also referred to as Antibody G-H (humanized)
Binds to human TCRVβ 20-1

| | | |
|---|---|---|
| SEQ ID NO: 1113 | VH-1 | QVQLVQSGAEVKKPGASVKVSCKASGFNIKSAYMHWVRQAP |
| | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: 1114 | VH-2 | QVQLVQSGAEVKKPGASVKVSCKASGFNIKSAYMHWVRQAP |
| | | GQEPGCMGRIDPATGKTKYAPKFQARVTMTADTSINTAYTEL |
| | | SSLRSEDTATYYCARSLNWDYGLDYWGQGTLVTVSS |
| SEQ ID NO: 1115 | VH-3 | QVQLVQSGAEVKKPGSSVKVSCKASGFNIKSAYMHWVRQAP |
| | | GQGLEWMGRIDPATGKTKYAPKFQARVTITADTSTNTAYMEL |
| | | SSLRSEDTAVYYCARSLNWDYGLDYWGQGTLVTVSS |
| SEQ ID NO: 1116 | VH-4 | QVQLVQSGAEVKKPGASVKVSCKASGFNIKSAYMHWVRQAP |
| | | GQRLEWMGRIDPATGKTKYAPKFQARVTITADTSANTAYMEL |
| | | SSLRSEDTAVYYCARSLNWDYGLDYWGQGTLVTVSS |

VL for MPB2D5 (humanized) also referred to as Antibody G-H (humanized)
Binds to human TCRVβ 20-1

| | | |
|---|---|---|
| SEQ ID NO: 1117 | VL-1 | EIVLTQSPATLSLSPGERATLSCRASKSVSILGTHLIHWYQQKP |
| | | GQAPRLLIYAASNLESGIPARFSGSGSETDFTLTISSLEPEDFAV |
| | | YFCQQSIEDPFGGGTKVEIK |
| SEQ ID NO: 1118 | VL-2 | EIVLTQSPATLSLSPGERATLSCRASKSVSILGTHLIHWYQQKP |
| | | GLAPRLLIYAASNLESGIPDRESGSGSETDFTLTISRLEPEDFAV |
| | | YFCQQSIEDPFGGGTKVEIK |
| SEQ ID NO: 1119 | VL-3 | EIVLTQSPGTLSLSPGERATLSCRASKSVSILGTHLIHWYQQKP |
| | | GQAPRLLIYAASNLESGIPDRFSGSGSETDFTLTISRLEPEDFAV |
| | | YFCQQSIEDPFGGGTKVEIK |

CAS1.1.3 (murine) also referred to herein as BJ1460; or Antibody H
Binds to human TCRVβ 27

| | | |
|---|---|---|
| SEQ ID NO: 1120 | HC CDR1 (Kabat) | DTYMY |
| SEQ ID NO: 1121 | HC CDR2 (Kabat) | RIDPANGNTKYDPKFQD |
| SEQ ID NO: 1122 | HC CDR3 (Kabat) | GSYYYAMDY |
| SEQ ID NO: 1123 | HC CDR1 (Chothia) | GFKTEDT |
| SEQ ID NO: 1124 | HC CDR2 (Chothia) | DPANGN |
| SEQ ID NO: 1122 | HC CDR3 (Chothia) | GSYYYAMDY |
| SEQ ID NO: 1125 | HC CDR1 (Combined) | GFKTEDTYMY |
| SEQ ID NO: 1121 | HC CDR2 (Combined) | RIDPANGNTKYDPKFQD |
| SEQ ID NO: 1122 | HC CDR3 (Combined) | GSYYYAMDY |
| SEQ ID NO: 1126 | LC CDR1 (Kabat) | RASESVDSYGNSFMH |
| SEQ ID NO: 1127 | LC CDR2 (Kabat) | RASNLES |
| SEQ ID NO: 1128 | LC CDR3 (Kabat) | QQSNEDPYT |
| SEQ ID NO: 3641 | LC CDR1 (Chothia) | SESVDSYGNSF |
| SEQ ID NO: 1127 | LC CDR2 (Chothia) | RASNLES |
| SEQ ID NO: 1128 | LC CDR3 (Chothia) | QQSNEDPYT |
| SEQ ID NO: 1126 | LC CDR1 (Combined) | RASESVDSYGNSFMH |
| SEQ ID NO: 1127 | LC CDR2 (Combined) | RASNLES |
| SEQ ID NO: 1128 | LC CDR3 (Combined) | QQSNEDPYT |
| SEQ ID NO: 1129 | VL | DIVLTQSPASLAVSLGQRATISCRASESVDSYGNSFMHWYQQK |
| | | PGQPPKLLIYRASNLESGIPARFSGSGSRTDFTLTINPVEADDVA |
| | | TYYCQQSNEDPYTFGGGTKLEIK |
| SEQ ID NO: 1130 | VH | EVQLQQSGAELVKPGASVKLSCTASGFKTEDTYMYWVKQRP |
| | | EQGLEWIGRIDPANGNTKYDPKFQDKATITADSSSNTAYLQLS |
| | | ISLPSEDTAVYYCARGSYYYAMDYWGQGTSVTVSS |

VH for CAS1.1.3 (humanized) also referred to as Antibody H-H (humanized)
Binds to human TCRVβ 27

| | | |
|---|---|---|
| SEQ ID NO: 1131 | VH-1 | QVQLVQSGAEVKKPGSSVKVSCKASGFKTEDTYMYWVRQAP |
| | | GQGLEWIGRIDPANGNTKYDPKFQDRATITADSSTNTAYMELS |
| | | SLRSEDTAVYYCARGSYYYAMDYWGQGTLVTVSS |
| SEQ ID NO: 1132 | VH-2 | QVQLVQSGAEVKKPGASVKVSCKASGFKTEDTYMYWVRQAP |
| | | GQRLEWIGRIDPANGNTKYDPKFQDRATITADSSANTAYMELS |
| | | SLRSEDTAVYYCARGSYYYAMDYWGQGTLVTVSS |
| SEQ ID NO: 1133 | VH-3 | EVQLVESGGGLVQPGGSLKLSCAASGFKTEDTYMYWVRQAS |
| | | GKGLEWIGRIDPANGNTKYDPKFQDRATISADSSKNTAYLQM |
| | | NSLKTEDTAVYYCARGSYYYAMDYWGQGTLVTVSS |
| SEQ ID NO: 1134 | VH-4 | EVQLVQSGAEVKKPGESLRISCKASGFKTEDTYMYWVRQMPG |
| | | KGLEWIGRIDPANGNTKYDPKFQDQATISADSSINTAYLQWSS |
| | | LKASDTAMYYCARGSYYYAMDYWGQGTLVTVSS |
| SEQ ID NO: 1135 | VH-5 | QVQLVQSGSELKKPGASVKVSCKASGFKTEDTYMYWVRQAP |
| | | GQGLEWIGRIDPANGNTKYDPKFQDRAVISADSSVNTAYLQIS |
| | | SLKAEDTAVYYCARGSYYYAMDYWGQGTLVTVSS |

VL for CAS1.1.3 (humanized) also referred to as Antibody H-H (humanized)
Binds to human TCRVβ 27

| | | |
|---|---|---|
| SEQ ID NO: 1136 | VL-1 | DIVLTQSPDSLAVSLGERATINCRASESVDSYGNSFMHWYQQK |
| | | PGQPPKLLIYRASNLESGVPDRFSGSGSRTDFTLTISSLQAEDVA |
| | | VYYCQQSNEDPYTFGQGTKLEIK |
| SEQ ID NO: 1137 | VL-2 | EIVLTQSPATLSLSPGERATLSCRASESVDSYGNSFMHWYQQK |
| | | PGQAPKLLIYRASNLESGIPARFSGSGSRTDFTLTISRLEPEDFA |
| | | VYYCQQSNEDPYTFGQGTKLEIK |
| SEQ ID NO: 1138 | VL-3 | DIQLTQSPSSLSASVGDRVTITCRASESVDSYGNSFMHWYQQK |
| | | PGQAPKLLIYRASNLESGVPSRFSGSGSRTDFTLTISSLQPEDVA |
| | | TYYCQQSNEDPYTFGQGTKLEIK |
| SEQ ID NO: 1139 | VL-4 | AIQLTQSPSSLSASVGDRVTITCRASESVDSYGNSFMHWYQQK |
| | | PGKAPKLLIYRASNLESGVPSRFSGSGSRTDFTLTISSLQPEDFA |
| | | TYYCQQSNEDPYTFGQGTKLEIK |
| SEQ ID NO: 1140 | VL-5 | EIVLTQSPDFQSVTPKEKVTITCRASESVDSYGNSFMHWYQQK |
| | | PDQSPKLLIYRASNLESGVPSRFSGSGSRTDFTLTINSLEAEDAA |
| | | TYYCQQSNEDPYTFGQGTKLEIK |

IMMU222 (marine) also referred to as BJ1461; or Antibody I
Binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3 (Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3 (Combined) | LQGSGFPLT |
| SEQ ID NO: 1151 | VL | ENVLTQSPAIMSASPGEKVTMTCSAGSSVSFMHWYQQKSSTSP |
| | | KLWIYDTSKLASGVPGRFSGSGSGNSFSLTISSMEAEDVAIYYC |
| | | LQGSGFPLTFGSGTKLEIK |
| SEQ ID NO: 1152 | VH | DVKLVESGEGLVKPGGSLKLSCAASGFTFRSYAMSWVRQTPE |
| | | KRLEWVAHISNGGDYIYYADTVKGRFTISRDNARNTLYLQMS |
| | | SLKSEDTAMYYCTRPSYYSDPWFFDVWGTGTTVTVSS |

VH for IMMU222 (humanized) also referred to as Antibody I-H
Binds to human TCRVβ 6-5, 6-6, 6-9

| | | |
|---|---|---|
| SEQ ID NO: 1153 | VH-1 | EVQLVESGGGLVQPGGSLRLSCAASGFTFRSYAMSWVRQAPG |
| | | KGLEWVAHISNGGDYIYYADTVKGRFTISRDNAKNSLYLQMN |
| | | SLRAEDTAVYYCTRPSYYSDPWFFDVWGQGTTVTVSS |
| SEQ ID NO: 1154 | VH-2 | QVQLVESGGGVVQPGRSLRLSCAASGFTFRSYAMSWVRQAPG |
| | | KGLEWVAHISNGGDYIYYADTVKGRFTISRDNSKNTLYLQMS |
| | | SLRAEDTAVYYCTRPSYYSDPWFFDVWGQGTTVTVSS |
| SEQ ID NO: 1155 | VH-3 | EVQLVESGGGLVQPGGSLRLSCAASGFTFRSYAMSWVRQAPG |
| | | KGLEWVAHISNGGDYIYYADTVKGRFTISRDNSKNTLYLQMN |
| | | SLRAEDTAVYYCTRPSYYSDPWFFDVWGQGTTVTVSS |
| SEQ ID NO: 1156 | VH-4 | QVQLVQSGSELKKPGASVKVSCKASQFTFRSYAMSWVRQAPG |
| | | QGLEWVAHISNGGDYIYYADTVKGRFVISRDNSVNTLYLQISS |
| | | LKAEDTAVYYCTRPSYYSDPWFFDVWGQGTTVTVSS |
| SEQ ID NO: 1157 | VH-5 | QVQLVQSGAEVKKPGASVKVSCKASGFTFRSYAMSWVRQAP |
| | | GQRLEWVAHISNGGDYIYYADTVKGRFTITRDNSANTLYMEL |
| | | SSLRSEDTAVYYCTRPSYYSDPWFFDVWGQGTTVTVSS |

VL for IMMU222 (humanized)) also referred to as Antibody I-H
Binds to human TCRVβ 6-5, 6-6, 6-9

| | | |
|---|---|---|
| SEQ ID NO: 1158 | VL-1 | ENVLTQSPATLSLSPGERATLSCSAGSSVSFMHWYQQKPGQAP |
| | | KLLIYDTSKLASGIPARFSGSGSGNDFTLTISSLEPEDFAVYYCL |
| | | QGSGFPLTFGQGTKLEIK |
| SEQ ID NO: 1159 | VL-2 | ENVLTQSPDFQSVTPKEKVTITCSAGSSVSFMHWYQQKPDQSP |
| | | KLLIYDTSKLASGVPSRFSGSGSGNDFTLTINSLEAEDAATYYC |
| | | LQGSGFPLTFGQGTKLEIK |
| SEQ ID NO: 1160 | VL-3 | DNQLTQSPSSLSASVGDRVTITCSAGSSVSFMHWYQQKPGKVP |
| | | KLLIYDTSKLASGVPSRFSGSGSGNDFTLTISSLQPEDVATYYC |
| | | LQGSGFPLTFGQGTKLEIK |
| SEQ ID NO: 1161 | VL-4 | ANQLTQSPSSLSASVGDRVTITCSAGSSVSFMHWYQQKPGKAP |
| | | KLLIYDTSKLASGVPSRFSGSGSGNDFTLTISSLQPEDFATYYCL |
| | | QGSGFPLTFGQGTKLEIK |
| SEQ ID NO: 1162 | VL-5 | DNVLTQSPDSLAVSLGERATINCSAGSSVSFMHWYQQKPGQPP |
| | | KLLIYDTSKLASGVPDRFSGSGSGNDFTLTISSLQAEDVAVYYC |
| | | LQGSGFPLTFGQGTKLEIK |

REA1062 (murine), also referred to as BJ1189 or as Antibody J
Binds to human TCRVβ 5-1

| | | |
|---|---|---|
| SEQ ID NO: 1163 | HC CDR1 (Kabat) | DYNIH |
| SEQ ID NO: 1164 | HC CDR2 (Kabat) | YINPYNGRTGYNQKFKA |
| SEQ ID NO: 1165 | HC CDR3 (Kabat) | WDGSSYFDY |
| SEQ ID NO: 1166 | HC CDR1 (Chothia) | GYTFTDYNIH |
| SEQ ID NO: 1167 | HC CDR2 (Chothia) | NPYNGR |
| SEQ ID NO: 1165 | HC CDR3 (Chothia) | WDGSSYFDY |
| SEQ ID NO: 1166 | HC CDR1 (Combined) | GYTFTDYNIH |
| SEQ ID NO: 1164 | HC CDR2 (Combined) | YINPYNGRTGYNQKFKA |
| SEQ ID NO: 1165 | HC CDR3 (Combined) | WDGSSYFDY |
| SEQ ID NO: 1168 | LC CDR1 (Kabat) | SASSSVSYMH |
| SEQ ID NO: 1169 | LC CDR2 (Kabat) | EISKLAS |
| SEQ ID NO: 1170 | LC CDR3 (Kabat) | QQWNYPLLT |
| SEQ ID NO: 1297 | LC CDR1 (Chothia) | SSSVSY |
| SEQ ID NO: 1169 | LC CDR2 (Chothia) | EISKLAS |
| SEQ ID NO: 1170 | LC CDR3 (Chothia) | QQWNYPLLT |
| SEQ ID NO: 1168 | LC CDR1 (Combined) | ISASSSVSYMH |
| SEQ ID NO: 1169 | LC CDR2 (Combined) | EISKLAS |
| SEQ ID NO: 1170 | LC CDR3 (Combined) | QQWNYPLLT |
| SEQ ID NO: 1171 | VL | EIVLTQSPAITAASLGQKVTITCSASSSVSYMHWYQQKSGTSPK |
| | | PWIYEISKLASGVPARFSGSGSGTSYSLTISSMEAEDAAIYYCQ |
| | | QWNYPLLTFGAGTKLELK |
| SEQ ID NO: 1172 | VH | EVQLQQSGPVLVKPGASVRMSCKASGYTFTDYNIHWVKQSH |
| | | GRSLEWVGYINPYNGRTGYNQKFKAKATLTVDKSSSTAYMD |
| | | LRSLTSEDSAVYYCARWDGSSYFDYWGQGTTLTVSS |

VH for REA1062 (humanized) also referred to as Antibody J-H
Binds to human TCRVβ 5-1

| | | |
|---|---|---|
| SEQ ID NO: 1173 | VH-1 | QVQLVQSGAEVKKPGSSVKVSCKASGYTFTDYNIHWVRQAPG |
| | | QGLEWVGYINPYNGRTGYNQKFKARATLTVDKSTSTAYMELS |
| | | SLRSEDTAVYYCARWDGSSYFDYWGQGTTVTVSS |
| SEQ ID NO: 1174 | VH-2 | QVQLVQSGAEVKKPGASVKVSCKASGYTFTDYNIHWVRQAP |
| | | GQGLEWVGYINPYNGRTGYNQKFKARATLTVDKSTSTAYME |
| | | LRSLRSDDMAVYYCARWDGSSYFDYWGQGTTVTVSS |
| SEQ ID NO: 1175 | VH-3 | QVQLVQSGAEVKKPGASVKVSCKASGYTFTDYNIHWVRQAT |
| | | GQGLEWVGYINPYNQRTGYNQKFKARATLTVNKSISTAYMEL |
| | | SSLRSEDTAVYYCARWDGSSYFDYWGQGTTVTVSS |
| SEQ ID NO: 1176 | VH-4 | EVQLVESGGGLVQPGRSLRLSCTASGYTFTDYNIHWVRQAPG |
| | | KGLEWVGYINPYNGRTGYNQKFKARATLSVDKSKSIAYLQM |
| | | NSLKTEDTAVYYCARWDGSSYFDYWGQGTTVTVSS |
| SEQ ID NO: 1177 | VH-5 | QVQLVQSGSELKKPGASVKVSCKASGYTFTDYNIHWVRQAPG |
| | | QGLEWVGYINPYNGRTGYNQKFKARAVLSVDKSVSTAYLQIS |
| | | SLKAEDTAVYYCARWDGSSYFDYWGQGTTVTVSS |

VL for REA1062 (humanized) also referred to as Antibody J-H
Binds to human TCRVβ 5-1

| | | |
|---|---|---|
| SEQ ID NO: 1178 | VL-1 | EIVLTQSPATLSLSPGERATLSCSASSSVSYMHWYQQKPGQAP |
| | | KLLIYEISKLASGIPARFSGSGSGTDYTLTISSLEPEDFAVYYCQ |
| | | QWNYPLLTFGQGTKLEIK |
| SEQ ID NO: 1179 | VL-2 | EIVLTQSPATLSLSPGERATLSCSASSSVSYMHWYQQKPGQAP |
| | | KLLIYEISKLASGIPARFSGSGSGTDYTLTISRLEPEDFAVYYCQ |
| | | QWNYPLLTFGQGTKLEIK |
| SEQ ID NO: 1180 | VL-3 | EIVLTQSPDFQSVTPKEKVTITCSASSSVSYMHWYQQKPDQSP |
| | | KLLIYEISKLASGVPSRFSGSGSGTDYTLTINSLEAEDAATYYC |
| | | QQWNYPLLTFGQGTKLEIK |
| SEQ ID NO: 1181 | VL-4 | DIQLTQSPSFLSASVGDRVTITCSASSSVSYMHWYQQKPGKAP |
| | | KLLIYEISKLASGVPSRFSGSGSGTEYTLTISSLQPEDFATYYCQ |
| | | QWNYPLLTFGQGTKLEIK |
| SEQ ID NO: 1182 | VL-5 | AIQLTQSPSSLSASVGDRVTITCSASSSVSYMHWYQQKPGKAP |
| | | KLLIYEISKLASGVPSRFSGSGSGTDYTLTISSLQPEDFATYYCQ |
| | | QWNYPLLTFGQGTKLEIK |
| SEQ ID NO: 1183 | VL-6 | AIRLTQSPFSLSASVGDRVTITCSASSSVSYMHWYQQKPAKAP |
| | | KLFIYEISKLASGVPSRFSGSGSGTDYTLTISSLQPEDFATYYCQ |
| | | QWNYPLLTFGQGTKLEIK |
| SEQ ID NO: 1184 | VL-7 | DIVLTQSPDSLAVSLGERATINCSASSSVSYMHWYQQKPGQPP |
| | | KLLIYEISKLASGVPDRFSGSGSGTDYTLTISSLQAEDVAVYYC |
| | | QQWNYPLLTFGQGTKLEIK |

JOVI-3 (murine), also referred to as BJ1187 or Antibody K
Binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3 (Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | ISANSTVQYIH |
| SEQ ID NO: 1192 | LC CDR2 (Combined) | TTSNLAS |
| SEQ ID NO: 1193 | LC CDR3 (Combined) | HQWSFYPT |
| SEQ ID NO: 1195 | VL | QIVLTQSPAIMSASLGEEIALTCSANSTVQYIHWYQQKSGTSPK |
| | | LLIYTTSNLASGVPSRFSGSGSGTFYSLTISSVEAEDAADYFCH |
| | | QWSFYPTFGGGTKLEIK |
| SEQ ID NO: 1196 | VH | QIQLQQSGPEVVKPGASVQISCKASGYTFSGSWMNWVKQRPG |
| | | KGLEWIGRIYPGDGDTDYSGKFKGRATLTADKSSSTAYMRLSS |
| | | LTSEDSAVYFCARSGYFNYVPVFDYWGQGTTLSVSS |

VH for JOVI-3 (humanized) also referred to as Antibody K-H
Binds to human TCRVβ 28

| | | |
|---|---|---|
| SEQ ID NO: 1197 | VH-1 | QIQLVQSGAEVKKPGASVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1198 | VH-2 | QIQLVQSGAEVKKPGSSVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1199 | VH-3 | EIQLVQSGAEVKKPGESLKISCKASGYTFSGSWMNWVRQMPG |
| | | KGLEWIGRIYPGDGDTDYSGKFKGQATLSADKSISTAYLQWSS |
| | | LKASDTAMYYCARSGYFNYVPVFDYWGQGTTVTVSS |
| SEQ ID NO: 1200 | VH-4 | QIQLVQSGSELKKPGASVKVSCKASGYTFSGSWMNWVRQAP |
| | | GQGLEWIGRIYPGDGDTDYSGKFKGRAVLSADKSVSTAYLQIS |
| | | SLKAEDTAVYYCARSGYFNYVPVFDYWGQGTTVTVSS |
| SEQ ID NO: 1201 | VH-5 | QIQLVQSGSELKKPGASVKVSCKASGYTFSGSWMNWVRQAP |
| | | GQGLEWIGRIYPGDGDTDYSGKFKGRAVLSADKSVSMAYLQI |
| | | SSLKAEDTAVYYCARSGYFNYVPVFDYWGQGTTVTVSS |
| SEQ ID NO: 1202 | VH-6 | EIQLVESGGGLVQPGRSLRLSCTASGYTFSGSWMNWVRQAPG |
| | | KGLEWIGRIYPGDGDTDYSGKFKGRATLSADKSKSIAYLQMN |
| | | SLKTEDTAVYYCARSGYFNYVPVFDYWGQGTTVTVSS |

VL for JOVI-3 (humanized) also referred to as Antibody K-H
Binds to human TCRVβ 28

| | | |
|---|---|---|
| SEQ ID NO: 1203 | VL-1 | EIVLTQSPATLSLSPGERATLSCSANSTVGYIHWYQQKPGQAP |
| | | KLLIYTTSNLASGIPARFSGSGSGTDYTLTISSLEPEDFAVYFCH |
| | | QWSFYPTFGQGTKLEIK |
| SEQ ID NO: 1204 | VL-2 | DIQLTQSPSFLSASVGDRVTITCSANSTVGYIHWYQQKPGKAP |
| | | KLLIYTTSNLASGVPSRFSGSGSGTEYTLTISSLQPEDFATYFCH |
| | | QWSFYPTFGQGTKLEIK |
| SEQ ID NO: 1205 | VL-3 | EIVLTQSPATLSLSPGERATLSCSANSTVGYIHWYQQKPGQAP |
| | | KLLIYTTSNLASGIPARFSGSGPGTDYTLTISSLEPEDFAVYFCH |
| | | QWSFYPTFGQGTKLEIK |
| SEQ ID NO: 1206 | VL-4 | DIVLTQSPDSLAVSLGERATINCSANSTVGYIHWYQQKPGQPP |
| | | KLLIYTTSNLASGVPDRFSGSGSGTDYTLTISSLQAEDVAVYFC |
| | | HQWSFYPTFGQGTKLEIK |
| SEQ ID NO: 1207 | IVL-5 | EIVLTQSPDFQSVTPKEKVTITCSANSTVGYIHWYQQKPDQSPK |
| | | LLIYTTSNLASGVPSRFSGSGSGTDYTLTINSLEAEDAATYFCH |
| | | QWSFYPTFGQGTKLEIK |

ZOE (marine), also referred to as BJ1538 or as Antibody L
Binds to human TCRVβ 4-1, 4-2, 4-3

| | | |
|---|---|---|
| SEQ ID NO: 1208 | HC CDR1 (Kabat) | DYYMY |
| SEQ ID NO: 1209 | HC CDR2 (Kabat) | TISGGGSYTYSPDSVKG |
| SEQ ID NO: 1210 | HC CDR3 (Kabat) | ERDIYYGNFNAMVY |
| SEQ ID NO: 1211 | HC CDR1 (Chothia) | GFTFSDY |
| SEQ ID NO: 1212 | HC CDR2 (Chothia) | SGGGSY |
| SEQ ID NO: 1210 | HC CDR3 (Chothia) | ERDIYYGNFNAMVY |
| SEQ ID NO: 1213 | HC CDR1 (Combined) | GFTFSDYYMY |
| SEQ ID NO: 1209 | HC CDR2 (Combined) | TISGGGSYTYSPDSVKG |
| SEQ ID NO: 1210 | HC CDR3 (Combined) | ERDIYYGNFNAMVY |
| SEQ ID NO: 1214 | LC CDR1 (Kabat) | RASKSVSTSGYSYMH |
| SEQ ID NO: 1215 | LC CDR2 (Kabat) | LASNLES |
| SEQ ID NO: 1216 | LC CDR3 (Kabat) | QHSRDLPWT |
| SEQ ID NO: 1217 | LC CDR1 (Chothia) | SKSVSTSQYSY |
| SEQ ID NO: 1215 | LC CDR2 (Chothia) | LASNLES |
| SEQ ID NO: 1216 | LC CDR3 (Chothia) | QHSRDLPWT |
| SEQ ID NO: 1214 | LC CDR1 (Combined) | RASKSVSTSGYSYMH |
| SEQ ID NO: 1215 | LC CDR2 (Combined) | LASNLES |
| SEQ ID NO: 1216 | LC CDR3 (Combined) | QHSRDLPWT |
| SEQ ID NO: 1218 | VL | DIVLTQSPVSLTVSLGQRATISCRASKSVSTSGYSYMHWYQQK |
| | | PGQPPKLLIYLASNLESGVPARFSGSGSGTDFTLNIHPVEEEDA |
| | | ATYYCQHSRDLPWTFGGGTKLEIK |
| SEQ ID NO: 1219 | VH | EVQLVESGGGLVKPGGSLKLSCAASGFTFSDYYMYWVRQTPE |
| | | KRLEWVATISGGGSYTYSPDSVKGRFTISRDNAKNNLYLOMSS |
| | | LRSEDTAMYFCARERDIYYGNFNAMVYWGRGTSVTVSS |

VH for ZOE (humanized) also referred to as Antibody L-H
Binds to human TCRVβ 4-1, 4-2, 4-3

| | | |
|---|---|---|
| SEQ ID NO: 1220 | VH-1 | EVQLLESGGGLVQPGGSLRLSCAASGFTFSDYYMYWVRQAPG |
| | | KGLEWVATISGGGSYTYSPDSVKGRFTISRDNSKNTLYLQMNS |
| | | LRAEDTAVYYCARERDIYYGNFNAMVYWGRGTLVTVSS |
| SEQ ID NO: 1221 | VH-2 | EVQLVESGGGLVQPGGSLRLSCAASGFTFSDYYMYWVRQAPG |
| | | KGLEWVATISGGGSYTYSPDSVKGRFTISRDNAKNSLYLQMNS |
| | | LRAEDTAVYYCARERDIYYGNFNAMVYWGRGTLVTVSS |
| SEQ ID NO: 1222 | VH-3 | QVQLVESGGGVVQPGRSLRLSCAASGFTFSDYYMYWVRQAP |
| | | GKGLEWVATISGGGSYTYSPDSVKGRFTISRDNSKNTLYLQM |
| | | NSLRAEDTAVYYCARERDIYYGNFNAMVYWGRGTLVTVSS |
| SEQ ID NO: 1223 | VH-4 | QVQLVESGGGLVKPGGSLRLSCAASGFTFSDYYMYWIRQAPG |
| | | KGLEWVATISGGGSYTYSPDSVKGRFTISRDNAKNSLYLQMNS |
| | | LRAEDTAVYYCARERDIYYGNFNAMVYWGRGTLVTVSS |

VL for ZOE (humanized) also referred to as Antibody L-H
Binds to human TCRVβ 4-1, 4-2, 4-3

| | | |
|---|---|---|
| SEQ ID NO: 1224 | VL-1 | EIVLTQSPGTLSLSPGERATLSCRASKSVSTSGYSYMHWYQQK |
| | | PGQAPRLLIYLASNLESGIPDRFSGSGSGTDFTLTISRLEPEDFA |
| | | VYYCQHSRDLPWTFGGGTKVEIK |
| SEQ ID NO: 1225 | VL-2 | EIVLTQSPATLSLSPGERATLSCRASKSVSTSGYSYMHWYQQK |
| | | PGQAPRLLIYLASNLESGIPARFSGSGSGTDFTLTISSLEPEDFAV |
| | | YYCQHSRDLPWTFGGGTKVEIK |
| SEQ ID NO: 1226 | VL-3 | DIQLTQSPSTLSASVGDRVTITCRASKSVSTSGYSYMHWYQQK |
| | | PGKAPKLLIYLASNLESGVPSRFSGSGSGTEFTLTISSLQPDDFA |
| | | TYYCQHSRDLPWTFGGGTKVEIK |
| SEQ ID NO: 1227 | VL-4 | AIQLTQSPSSLSASVGDRVTITCRASKSVSTSGYSYMHWYQQK |
| | | PGKAPKLLIYLASNLESGVPSRFSGSGSGTDFTLTISSLQPEDFA |
| | | TYYCQHSRDLPWTFGGGTKVEIK |

Anti-TCRvb19 (murine), also referred to as BJ1465; or Antibody M
Binds to human TCRVβ 19

| | | |
|---|---|---|
| SEQ ID NO: 1229 | HC CDR1 (Kabat) | GYFWN |
| SEQ ID NO: 1230 | HC CDR2 (Kabat) | YISYDGSNNYNPSLKN |
| SEQ ID NO: 1231 | HC CDR3 (Kabat) | PSPGTGYAVDY |
| SEQ ID NO: 1232 | HC CDR1 (Chothia) | GYSITSGY |
| SEQ ID NO: 1233 | HC CDR2 (Chothia) | SYDGSN |
| SEQ ID NO: 1231 | HC CDR3 (Chothia) | PSPGTGYAVDY |
| SEQ ID NO: 1234 | HC CDR1 (Combined) | GYSITSGYFWN |
| SEQ ID NO: 1230 | HC CDR2 (Combined) | YISYDGSNNYNPSLKN |
| SEQ ID NO: 1231 | HC CDR3 (Combined) | PSPGTGYAVDY |
| SEQ ID NO: 1235 | LC CDR1 (Kabat) | RSSQSLVHSNGNTYLH |
| SEQ ID NO: 1236 | LC CDR2 (Kabat) | KVSNRFS |
| SEQ ID NO: 1237 | LC CDR3 (Kabat) | SQSTHVPFT |
| SEQ ID NO: 1238 | LC CDR1 (Chothia) | SQSLVHSNGNTY |
| SEQ ID NO: 1236 | LC CDR2 (Chothia) | KVSNRFS |
| SEQ ID NO: 1237 | LC CDR3 (Chothia) | SQSTHVPFT |
| SEQ ID NO: 1235 | LC CDR1 (Combined) | RSSQSLVHSNGNTYLH |
| SEQ ID NO: 1236 | LC CDR2 (Combined) | KVSNRFS |
| SEQ ID NO: 1237 | LC CDR3 (Combined) | SQSTHVPFT |
| SEQ ID NO: 1239 | VL | NVVMTQTPLSLPVSLGDQASISCRSSQSLVHSNGNTYLHWYLQ |
| | | KPGQSPKFLIYKVSNRFSGVPDRFSGGGSGTEFTLKISRVEAED |
| | | LGVYFCSQSTHVPFTFGSGTKLEIK |
| SEQ ID NO: 1240 | VH | NVQLQESGPGVKPSQSLSLTCSVAGYSITSGYFWNWIRQFPG |
| | | NKLEWMGYISYDGSNNYNPSLKNRISITRDTSKNQFFLKLNSV |
| | | TTEDTATYYCASPSPGTGYAVDYWGQGTSVTVSS |

VH for Anti-TCRvb19 (humanized) also referred to as Antibody M-H
Binds to human TCRVβ 19

| | | |
|---|---|---|
| SEQ ID NO: 1241 | VH-1 | QVQLQESGPGLVKPSETLSLTCTVSGYSITSGYFWNWIRQPPG |
| | | KGLEWIGYISYDGSNNYNPSLKNRVTISRDTSKNQFSLKLSSVT |
| | | AADTAVYYCASPSPGTGYAVDYWGQGTLVTVSS |
| SEQ ID NO: 1242 | VH-2 | QVQLQESGPGLVKPSETLSLTCTVSGYSITSGYFWNWIRQPPG |
| | | KGLEWIGYISYDGSNNYNPSLKNRVTISRDTSKNQFSLKLSSVT |
| | | AADTAVYYCASPSPGTGYAVDYWGQGTLVTVSS |
| SEQ ID NO: 1243 | VH-3 | QVQLVESGGGLVQPGGSLRLSCSVSGYSITSGYFWNWVRQAP |
| | | GKGLEWVGYISYDGSNNYNPSLKNRFTISRDTSKNTFYLQMNS |
| | | LRAEDTAVYYCASPSPGTGYAVDYWGQGTLVTVSS |

VL for Anti-TCRvb19 (humanized) also referred to as Antibody M-H
Binds to human TCRVβ 19

| | | |
|---|---|---|
| SEQ ID NO: 1244 | VL-1 | VVMTQSPGTLSLSPGERATLSCRSSQSLVHSNGNTYLHWYQQ |
| | | KPGQAPRFLIYKVSNRFSGIPDRFSGSGSGTDFTLTISRLEPEDF |
| | | AVYFCSQSTHVPFTFGQGTKLEIK |
| SEQ ID NO: 1245 | VL-2 | EVVMTQSPATLSLSPGERATLSCRSSQSLVHSNGNTYLHWYQ |
| | | QKPGQAPRFLIYKVSNRFSGIPARFSGSGSGTDFTLTISSLEPED |
| | | FAVYFCSQSTHVPFTFGQGTKLEIK |
| SEQ ID NO: 1246 | VL-3 | EVVMTQSPATLSVSPGERATLSCRSSQSLVHSNGNTYLHWYQ |
| | | QKPGQAPRFLIYKVSNRFSGIPARFSGSGSGTEFTLTISSLQSED |
| | | FAVYFCSQSTHVPFTFGQGTKLEIK |
| SEQ ID NO: 1247 | VL-4 | DVQMTQSPSSLSASVGDRVTITCRSSQSLVHSNGNTYLHWYQ |
| | | QKPGKAPKFLIYKVSNRFSGVPSRFSGSGSGTDFTFTISSLQPED |
| | | IATYFCSQSTHVPFTFGQGTKLEIK |

BL37.2 (murine), also referred to as BJ1539 or Antibody N
Binds to human TCRVβ 9

| | | |
|---|---|---|
| SEQ ID NO: 1248 | HC CDR1 (Kabat) | DYIVH |
| SEQ ID NO: 1249 | HC CDR2 (Kabat) | WINTYTGTPTYADDFEG |
| SEQ ID NO: 1250 | HC CDR3 (Kabat) | SWRRGIRGIGFDY |
| SEQ ID NO: 1251 | HC CDR1 (Chothia) | GYTFTDY |
| SEQ ID NO: 1252 | HC CDR2 (Chothia) | NTYTGT |
| SEQ ID NO: 1250 | HC CDR3 (Chothia) | SWRRGIRGIGFDY |
| SEQ ID NO: 1253 | HC CDR1 (Combined) | GYTFTDYIVH |
| SEQ ID NO: 1249 | HC CDR2 (Combined) | WINTYTGTPTYADDFEG |
| SEQ ID NO: 1250 | HC CDR3 (Combined) | SWRRGIRGIGFDY |
| SEQ ID NO: 1254 | LC CDR1 (Kabat) | KASKSINKYLA |
| SEQ ID NO: 1255 | LC CDR2 (Kabat) | DGSTLQS |
| SEQ ID NO: 1256 | LC CDR3 (Kabat) | QQHNEYPPT |
| SEQ ID NO: 1257 | LC CDR1 (Chothia) | SKSINKY |
| SEQ ID NO: 1255 | LC CDR2 (Chothia) | DQSTLQS |
| SEQ ID NO: 1256 | LC CDR3 (Chothia) | QQHNEYPPT |
| SEQ ID NO: 1254 | LC CDR1 (Combined) | KASKSINKYLA |
| SEQ ID NO: 1255 | LC CDR2 (Combined) | DGSTLQS |
| SEQ ID NO: 1256 | LC CDR3 (Combined) | QQHNEYPPT |
| SEQ ID NO: 1258 | VL | DVQMTQSPYNLAASPGESVSINCKASKSINKYLAWYQQKPGK |
| | | PNKLLIYDGSTLQSGIPSRFSGSGSGTDFTLTIRGLEPEDFGLYY |
| | | CQQHNEYPPTFGAGTKLELK |
| SEQ ID NO: 1259 | VH | QLQLVQSGPELREPGESVKISCKASGYTFTDYIVHWVKQAPGK |
| | | GLKWMGWINTYTGTPTYADDFEGRFVFSLEASASTANLQISNL |
| | | KNEDTATYFCARSWRRGIRGIGFDYWGQGVMVTVSS |

VH for BL37.2 (humanized) also referred to as Antibody N-H
Binds to human TCRVβ 9

| | | |
|---|---|---|
| SEQ ID NO: 1260 | VH-1 | QLQLVQSGAEVKKPGASVKVSCKASGYTFTDYIVHWVRQAP |
| | | GQGLEWMGWINTYTGTPTYADDFEGWVTMTLDASISTAYME |
| | | LSRLRSDDTAVYYCARSWRRGIRGIGFDYWGQGTMVTVSS |
| SEQ ID NO: 1261 | VH-2 | QLQLVQSGAEVKKPGASVKVSCKASGYTFTDYIVHWVRQAP |
| | | GQGLEWMGWINTYTGTPTYADDFEGRVTMTLDASTSTAYME |
| | | LSSLRSEDTAVYYCARSWRRGIRGIGFDYWGQGTMVTVSS |
| SEQ ID NO: 1262 | VH-3 | QLQLVQSGAEVKKPGASVKVSCKASGYTFTDYIVHWVRQAP |
| | | GQRLEWMGWINTYTGTPTYADDFEGRVTITLDASASTAYMEL |
| | | SSLRSEDMAVYYCARSWRRGIRGIGFDYWGQGTMVTVSS |
| SEQ ID NO: 1263 | VH-4 | QLQLVQSGAEVKKPGASVKVSCKASGYTFTDYIVHWVRQAT |
| | | GQGLEWMGWINTYTGTPTYADDFEGRVTMTLNASISTAYME |
| | | LSSLRSEDTAVYYCARSWRRGIRGIGFDYWGQGTMVTVSS |

VL for BL37.2 (humanized) also referred to as Antibody N-H
Binds to human TCRVβ 9

| | | |
|---|---|---|
| SEQ ID NO: 1264 | VL-1 | EVVMTQSPGTLSLSPGERATLSCKASKSINKYLAWYQQKPGQ |
| | | APRLLIYDGSTLQSGIPDRFSGSGSGTDFTLTISRLEPEDFAVYY |
| | | CQQHNEYPPTFGQGTKLEIK |
| SEQ ID NO: 1265 | VL-2 | EVVMTQSPATLSLSPGERATLSCKASKSINKYLAWYQQKPGQ |
| | | APRLLIYDGSTLQSGIPARFSGSGSGTDFTLTISSLEPEDFAVYY |
| | | CQQHNEYPPTFGQGTKLEIK |
| SEQ ID NO: 1266 | VL-3 | DVQMTQSPSSLSASVGDRVTITCKASKSINKYLAWYQQKPGK |
| | | APKLLIYDGSTLQSGVPSRFSGSGSGTDFTLTISSLQPEDFATYY |
| | | CQQHNEYPPTFGQGTKLEIK |
| SEQ ID NO: 1267 | VL-4 | AVRMTQSPSSFSASTGDRVTITCKASKSINKYLAWYQQKPGK |
| | | APKLLIYDGSTLQSGVPSRFSGSGSGTDFTLTISCLQSEDFATYY |
| | | CQQHNEYPPTFGQGTKLEIK |

IG125 (murine) binds to TRVβ 11-2; also referred to as Antibody O

| | | |
|---|---|---|
| SEQ ID NO: 1268 | HC CDR1 (Kabat) | NYGVH |
| SEQ ID NO: 1269 | HC CDR2 (Kabat) | VIWSDGSTDYDTAFIS |
| SEQ ID NO: 1270 | HC CDR3 (Kabat) | RAVVADEDY |
| SEQ ID NO: 1271 | HC CDR1 (Chothia) | GFSLTN |
| SEQ ID NO: 1272 | HC CDR2 (Chothia) | VIWSDGSTD |
| SEQ ID NO: 1270 | HC CDR3 (Chothia) | RAVVADFDY |
| SEQ ID NO: 1273 | HC CDR1 (combined) | GFSLTNYGVH |
| SEQ ID NO: 1269 | HC CDR2 (combined) | VIWSDGSTDYDTAFIS |
| SEQ ID NO: 1270 | HC CDR3 (combined) | RAVVADFDY |
| SEQ ID NO: 1274 | VH | QVQLKQSGPGLLQPSQSLSITCTVSQFSLTNYGVHWVRQSPGK |
| | | GLEWLGVIWSDGSTDYDTAFISRLSISKDNSKSQVFFKLNSLQ |
| | | ADDTAIYYCARRAVVADFDYWGQGTTLTVSS |
| SEQ ID NO: 1275 | LC CDR1 (Kabat) | KASKEVTIFGSISALH |
| SEQ ID NO: 1276 | LC CDR2 (Kabat) | NGAKLES |
| SEQ ID NO: 1277 | LC CDR3 (Kabat) | LQNKEVPFT |
| SEQ ID NO: 1275 | LC CDR1 (Chothia) | KASKEVTIFGSISALH |
| SEQ ID NO: 1276 | LC CDR2 (Chothia) | NGAKLES |
| SEQ ID NO: 1277 | LC CDR3 (Chothia) | LQNKEVPFT |
| SEQ ID NO: 1275 | LC CDR1 (combined) | KASKEVTIFGSISALH |
| SEQ ID NO: 1276 | LC CDR2 (combined) | NGAKLES |
| SEQ ID NO: 1277 | LC CDR3 (combined) | LQNKEVPFT |
| SEQ ID NO: 1278 | VL | DIVLTQSPASLAVSLGQKATISCKASKEVTIFGSISALHWYQQ |
| | | KPGQPPKLIYNGAKLESGVSARFSDSGSQNRSPFGNQLSFTLTI |
| | | APVEADDAATYYCLQNKEVPFTFGSGTKLEIK |

VL for IG125 (humanized) also referred to as Antibody O-H
binds to TRVβ 11-2

| | | |
|---|---|---|
| SEQ ID NO: 1279 | VL-1 | DIVLTQSPDSLAVSLGERATINCKASKEVTIFGSISALHWYQQ |
| | | KPGQPPKLLYNGAKLESGVSARFGVPDRFSRSGSGLDFTLTIS |
| | | SLQAEDVAVYYCLQNKEVPFTFGQGTKLEIK |
| SEQ ID NO: 1280 | VL-2 | EIVLTQSPDFQSVTPKEKVTITCKASKEVTIFGSISALHWYQQK |
| | | PDQSPKLLYNGAKLESGVSARFGVPSRFSRSGSGLDFTLTINS |
| | | LEAEDAATYYCLQNKEVPFTFGQGTKLEIK |
| SEQ ID NO: 1281 | VL-3 | AIQLTQSPSSLSASVGDRVTITCKASKEVTIFGSISALHWYQQK |
| | | PGKAPKLLYNGAKLESGVSARFGVPSRFSRSGSGLDFTLTISS |
| | | LQPEDFATYYCLQNKEVPFTFGQGTKLEIK |
| SEQ ID NO: 1282 | VL-4 | DIVLTQTPLSLSVTPGQPASISCKASKEVTIFGSISALHWYLQK |
| | | PGQPPKLLYNGAKLESGVSARFGVPDRFSRSGSGLDFTLKISR |
| | | VEAEDVGVYYCLQNKEVPFTFGQGTKLEIK |

VH for IG125 (humanized) also referred to as Antibody O-H
binds to TRVβ 11-2

| | | |
|---|---|---|
| SEQ ID NO: 1283 | VH-1 | QVTLKESGPVLVKPTETLTLTCTVSGFSLTNYGVHWVRQPPG |
| | | KALEWLGVIWSDGSTDYDTAFISRLTISKDNSKSQVVLTMTN |
| | | MDPVDTATYYCARRAVVADFDYWGQGTTVTVSS |
| SEQ ID NO: 1284 | VH-2 | QVQLQESGPGLVKPSGTLSLTCAVSGFSLTNYGVHWVRQPPG |
| | | KGLEWLGVIWSDGSTDYDTAFISRLTISKDNSKSQVSLKLSSV |
| | | TAADTAVYYCARRAVVADFDYWGQGTTVTVSS |
| SEQ ID NO: 1285 | VH-3 | QVQLQQSGPGLVKPSQTLSLTCAVSGFSLTNYGVHWVRQSPS |
| | | RGLEWLGVIWSDGSTDYDTAFISRLTINKDNSKSQVSLQLNS |
| | | VTPEDTAVYYCARRAVVADFDYWGQGTTVTVSS |
| SEQ ID NO: 1286 | VH-4 | EVQLVESGGGLVQPGPSLRLSCTVSGFSLTNYGVHWVRQAP |
| | | GKGLEWLGVIWSDGSTDYDTAFISRLTISKDNSKSIVYLQMNS |
| | | LKTEDTAVYYCARRAVVADFDYWGQGTTVTVSS |
| SEQ ID NO: 1287 | IVH-5 | EVQLVQSGAEVKKPGESLRISCKVSGFSLTNYGVHWVRQMP |
| | | GKGLEWLGVIWSDGSTDYDTAFISQLTISKDNSISTVYLQWSS |
| | | LKASDTAMYYCARRAVVADFDYWGQGTTVTVSS |

MR5-2 (marine), Binds to human TCRVβ 13-2

| | | |
|---|---|---|
| SEQ ID NO: 1376 | SCFV (VH + VL) | QVQLQQSGTELMKPGASVKISCKASGYTFSNYWIEWIKQRPG |
| | | HGLEWVGEILPGAGPTNYNEKFKGKATFTADSSSNTAYMQL |
| | | SSLTSEDSAVYYCARTDYDYDWFAYWGQGTLVTVSAGGGG |
| | | SGGGGSGGGGSGGGGSDIVMSQSPSSLAVSVGEKVTMSCKSS |
| | | QSLLYSGNQKNYLAWYQQKPGQSPKLLIYWASTRESGVPDR |
| | | FTGSGSGTDFTLTINSVKAEDLTVYYCQQYYGYPRTFGGGTK |
| | | VEIK |

TABLE 14
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 15
Amino acid sequences of exemplary variable regions of anti-BCMA antibodies.

| SEQ ID | | |
|---|---|---|
| NO | Description | Sequence |
| 3439 | 83A10 VH | EVQLLESGGGLVQPGGSLRLSCAASGFTFSSYAMSWVRQAPGKGLEWVSAISG |
| | | SGGSTYYADSVKGRFTISRDNSKNTLYLQMNSLRAEDTAVYYCAKVLGWFDY |
| | | WGQGTLVTVSS |
| 3440 | 83A10 VL | EIVLTQSPGTLSLSPGERATLSCRASQSVSSSYLAWYQQKPGQAPRLLIYGASSR |
| | | ATGIPDRFSGSGSGTDFTLTISRLEPEDFAVYYCQQYGYPPDFTFGQGTKVEIK |
| 3441 | 17A5 VH | EVQLLESGGGLVQPGGSLRLSCAASGFTFSSYAMSWVRQAPGKGLEWVSAISG |
| | | SGGSTYYADSVKGRFTISRDNSKNTLYLQMNSLRAEDTAVYYCAKVAPYFAPF |
| | | DYWGQGTLVTVSS |
| 3442 | 17A5 VL | EIVLTQSPGTLSLSPGERATLSCRASQSVSSSYLAWYQQKPGQAPRLLIYGASSR |
| | | ATGIPDRFSGSGSGTDFTLTISRLEPEDFAVYYCQQYGNPPLYTFGQGTKVEIK |
| 3443 | 13A4 VH | EVQLVQSGAEVKKPGESLKISCKGSGYSFTSYWIGWVRQMPGKGLEWMGIIYP |
| | | GDSDTRYSPSFQGQVTISADKSISTAYLQWSSLKASDTAMYYCARNGYLGDYW |
| | | GQGTLVTVSS |
| 3444 | 13A4 VL | DIVMTQSPLSLPVTPGEPASISCRSSQSLLHSNGYNYLDWYLQKPGQSPQLLIYL |
| | | GSNRASGVPDRFSGSGSGTDFTLKISRVEAEDVGVYYCMQAMQIPTFGQGTKV |
| | | EIK |
| 3445 | J22.9-xi VH | QVQLQQSGGGLVQPGGSLKLSCAASGIDFSRYWMSWVRRAPGKGLEWIGEINP |
| | | DSSTINYAPSLKDKFIISRDNAKNTLYLQMSKVRSEDTALYYCASLYYDYGDA |
| | | MDYWGQGTSVTVSS |
| 3446 | J22.9-xi VL | DIVMTQSQRFMTTSVGDRVSVTCKASQSVDSNVAWYQQKPRQSPKALIFSASL |
| | | RFSGVPARFTGSGSGTDFTLTISNLQSEDLAEYFCQQYNNYPLTFGAGTKLELK |
| | | R |
| 3447 | 2A1 VH | EVQLVESGGGLVKPGGSLRLSCAASGFTFGDYALSWFRQAPGKGLEWVGVSR |
| | | SKAYGGTTDYAASVKGRFTISRDDSKSTAYLQMNSLKTEDTAVYYCASSGYSS |
| | | GWTPFDYWGQGTLVTVSS |
| 3448 | 2A1 VL | QSVLTQPPSASGTPGQRVTISCSGSSSNIGSNTVNWYQQLPGTAPKLLIFNYHQR |
| | | PSGVPDRFSGSKSGSSASLAISGLQSEDEADYYCAAWDDSLNGWVFGGGTKLT |
| | | VLG |

### Examples

The present disclosure will be more specifically illustrated by the following Examples. However, it should be understood that the present disclosure is not limited by these examples in any manner.

### Example 1: Humanization of α-TRBV6-5 Antibody Clone Anti-body A

The germline for the mouse α-TCRβ antibody clone Antibody A VH and VL were assigned using IMGT nomenclature, with CDR regions defined by a combined Kabat and Chothia classification, SEQ ID NO: 1 and SEQ ID NO: 2 are the Antibody A VH and VL sequences respectively where the VH germline is mouse IGHV1S12*01 and the VL germline is mouse IGKV6-15*01, SEQ ID NOs: 3-5 are the Antibody A VH CDR regions 1-3 respectively and SEQ ID NOs: 6-8 correspond to the VL CDR regions 1-3 (as described in Table 1).

Humanization of the Antibody A VH and VL sequences was done separately using similar methodology. Amino acids positions were identified in the framework regions which were important for the success of CDR grafting. Human germline sequences were identified which preserved the necessary residues and contained a high amount of overall identity. When the human germline framework sequence did not contain a matching important amino acid, it was back mutated to match the mouse sequence CDR regions were grafted onto the human germline unchanged. The Antibody A VH was humanized into human IGHV1-69*01 and the Antibody A VL was humanized into IGKV1-17*01 and IGKV1-27*01. All 3 humanized sequences were confirmed to contain no introduced potential negative post translational modification sites such as NG, DG, NS, NN, DS, NT, NXS, or NXT as a result of the humanization process, SEQ ID NO: 9 is the humanized Antibody A-H.1 VH and SEQ ID NOs: 10 and 11 are the humanized VL IGKV1-17*01 and IGKV1-27*01 germlines respectively (as described in Table 1). FIGS. 2A and 2B show the murine and humanized sequences with annotations depicting the CDR and framework regions (FR).

### Example 2: Humanization of α-TRBV12-3 and TRBV12-4 Antibody Clone Antibody B

The germline for the mouse α-TCRB antibody clone Antibody B VH and VL were assigned using IMGT nomenclature, with CDR regions defined by a combined Kabat and Chothia classification, SEQ ID NO: 15 and SEQ ID NO: 16 are the Antibody B VH and VL sequences respectively where the VH germline is mouse IGHV5-17*02 and the VL germline is mouse IGKV4-50*01, SEQ ID NOs: 17-19 are the B-H VH CDR regions 1-3 respectively and SEQ ID NOs: 20-22 are the B-H VL CDR regions 1-3 (as described in Table 2).

The method applied to humanize Antibody A described in Example 1 was used to humanize Antibody B. The Antibody B VH was humanized into human IGHV3-30*01, IGHV3-48*01, and IGHV3-66*01 and the Antibody B VL was humanized into human IGKV1-9*01, IGKV1-39*01, IGKV3-15*01, IGLV1-47*01 and IGLV3-10*01, SEQ ID NOs: 23-25 are the B-H.1A, B-H.1B, and B-H.1C humanized heavy chains and SEQ ID NOs: 26-30 are the B-H.1D, B-H1E, B-H.1F, B-H.1G and B-H.1H humanized light chains (as described in Table 2). FIGS. 3A and 3B show the murine and humanized sequences with annotations depicting the CDR and framework regions (FR).

### Example 3: Characteristics of Anti-TCRβV Antibodies

### Introduction

Current bispecific constructs designed to redirect T cells to promote tumor cell lysis for cancer immunotherapy typically utilize single chain variable fragments (scFvs) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that, e.g., low “activating” doses of anti-CD3e mAb can cause long-term T cell dysfunction and exert immunosuppressive effects. In addition, anti-CD3e mAbs bind to all T cells and thus activate equally all T cells, which has been associated with the first dose side effects of anti-CD3e mAbs that result from massive T cell activation. These large number of activated T cells secrete substantial amounts of cytokines, the most important of which is Interferon gamma (IFNg or IFNγ). This excess amount of IFNγ in turn, e.g., activates macrophages which then can overproduce proinflammatory cytokines such as IL-1, IL-6 and TNF-alpha, causing a “cytokine storm” known as the cytokine release syndrome (CRS). Thus, it might be advantageous to develop antibodies that are capable of binding and activating only a subset of necessary effector T cells to reduce the CRS.

### Results

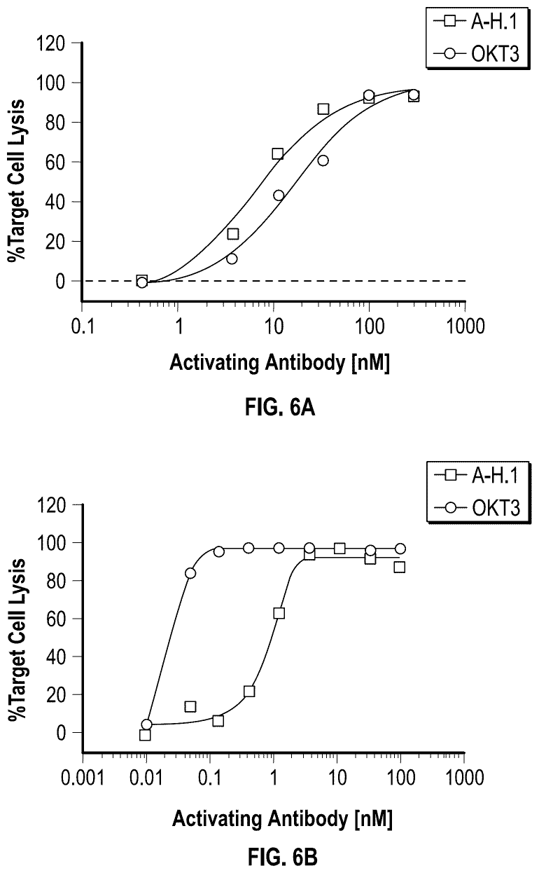

To that end, antibodies directed to the variable chain of the beta subunit of TCR (TCR Vb) were identified. These anti-TCR Vb antibodies bind and activate a subset of T cells, but with, e.g., no or markedly reduced CRS. Using plate-bound anti-TCR Vb13.1 mAbs (A-H.1 and A-H.2) it was shown that a population of T cells, defined by positive staining with A-H.1, can be expanded (from ˜5% of T cells on day 0 to almost 60% of total T cells on day 6 of cell culture) (FIGS. SA-5C). For this experiment, human CD3+ T cells were isolated using magnetic-bead separation (negative selection) and activated with immobilized (plate-coated) A-H.1 or OKT3 (anti-CD3e) antibodies at 100 nM for 6 days. The expanded Vb13.1+ T cells display cytolytic activity against transformed cell line RPMI-8226 when co-cultured with purified CD3+ T cells (FIGS. 6A-6B).

Next, the ability of PBMCs activated by anti-TCR Vβ antibodies to produce cytokines was assessed. The cytokine production of PBMCs activated with anti-TCR Vβ antibodies was compared to the cytokine production of PBMCs activated with: (i) anti-CD3e antibodies (OKT3 or SP34-2); (ii) anti-TCR V alpha (TCR VA) antibodies including anti-TCR VA 12.1 antibody 6D6.6, anti-TCR VA24JA 18 antibody 6B11; (iii) anti-TCR alpha beta antibody T10B9; and/or (iv) isotype control (BGM0109). The anti-TCR VB antibodies tested include: humanized anti-TCRVB 13.1 antibodies (A-H.1, or A-H.2), murine anti-TCR VB5 antibody E, murine anti-TCR VB8.1 antibody B, and murine anti-TCR VB12 antibody D. BGM0109 comprises the amino acid sequence of

(SEQ ID NO: 3282)
METDTLLLWVLLLWVPGSTGGLNDIFEAQKIEWHEGGGGSEPRTDTDTCPNPPDPCPTCPTPDLLGG
PSVFIFPPKPKDVLMISLTPKITCVVVDVSEEEPDVQFNWYVNNVEDKTAQTETRQRQYNSTYRVVS
VLPIKHQDWMSGKVFKCKVNNNALPSPIEKTISKPRGQVRVPQIYTFPPPIEQTVKKDVSVTCLVTGF
LPQDIHVEWESNGQPQPEQNYKNTQPVLDSDGSYFLYSKLNVPKSRWDQGDSFTCSVIHEALHNHH
MTKTISRSLGNGGGGS.

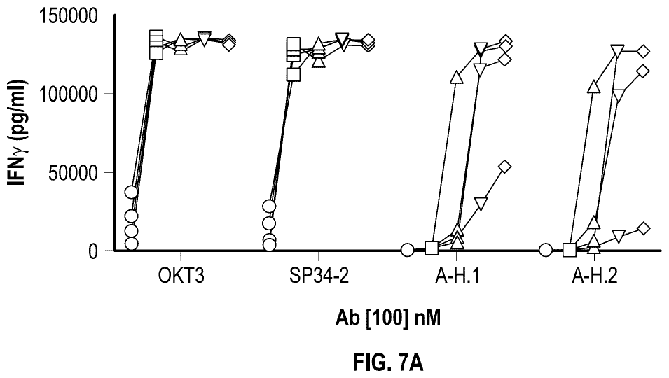

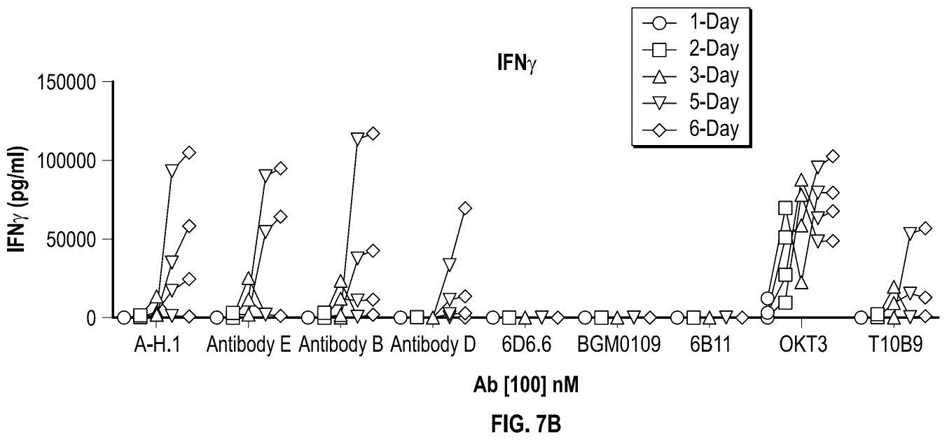

As shown in FIG. 7A, when plate-bound A-H.1 or A-H.2, or anti-CD3e antibodies (OKT3 or SP34-2) were used to activate human PBMCs, the T cell cytokine IFNg was induced (FIG. 7A). All anti-TCR VB antibodies tested had a similar effect on the production of IFNg (FIG. 7B). The anti-TCR VA antibodies did not induce similar IFNg production.

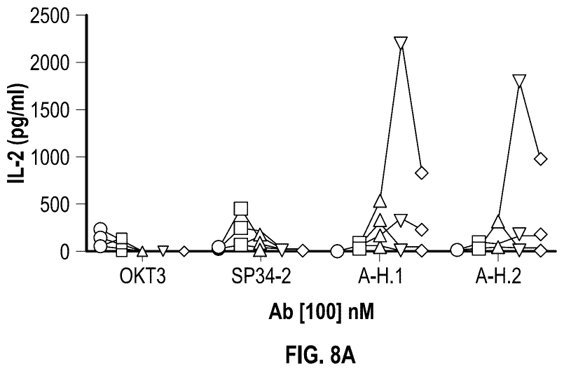

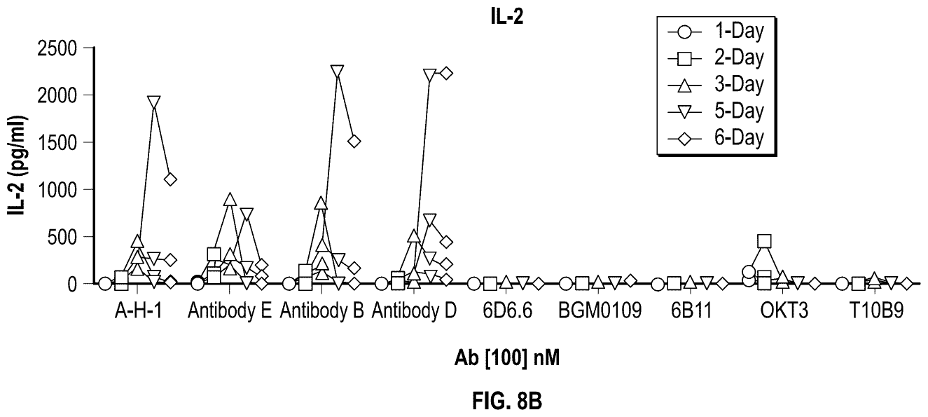

With respect to IL-2 production, PBMCs activated with A-H.1 and A-H.2 resulted in increased IL-2 production (FIG. 8A) with delayed kinetics (FIG. 8B) as compared to PBMCs activated with anti-CD3e antibodies (OKT3 or SP34-2). FIG. 8B shows that anti-TCR Vβ antibody activated PBMCs demonstrate peak production of IL-2 at Day 5 or Day 6 post-activation (incubation with plate-coated antibodies). In contrast. IL-2 production in PBMCs activated with OKT3 peaked at day 2 post-activation. As with IFNG, the IL-2 effect (e.g., enhanced production of IL-2 and delayed kinetics) was similar across all anti-TCR Vβ antibodies tested (FIG. 8B).

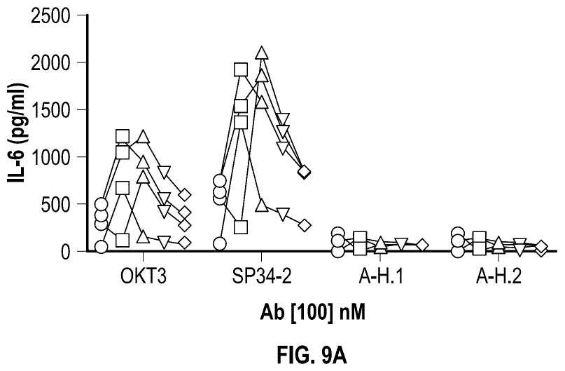

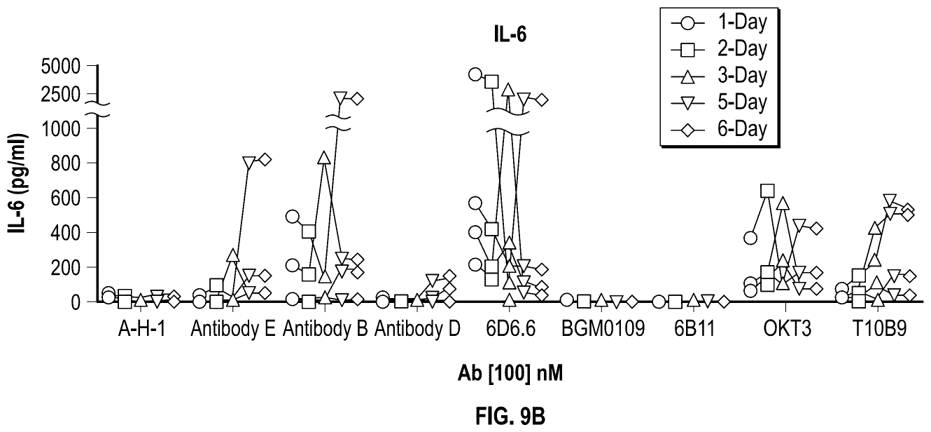

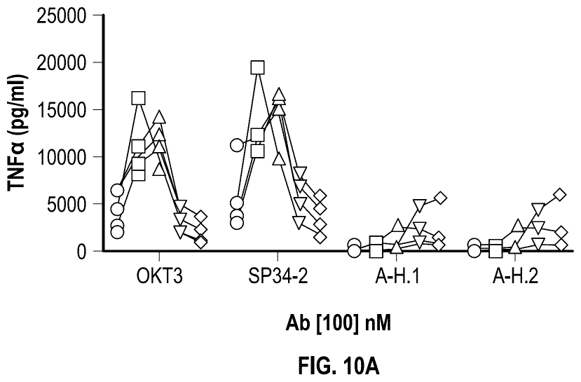

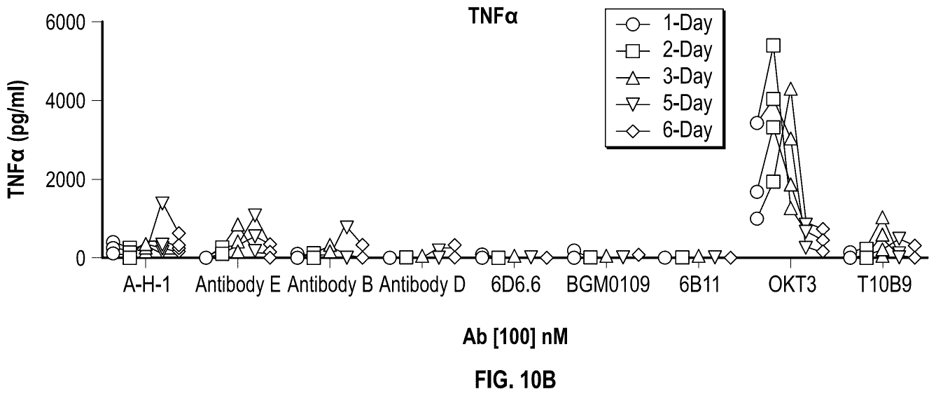

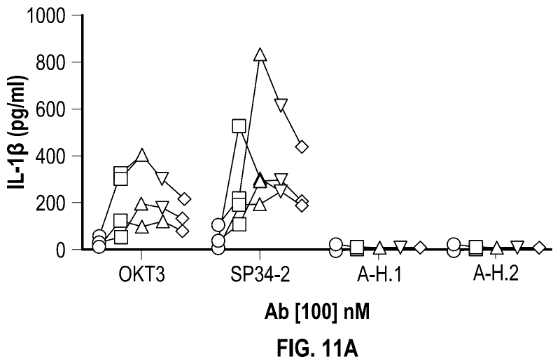

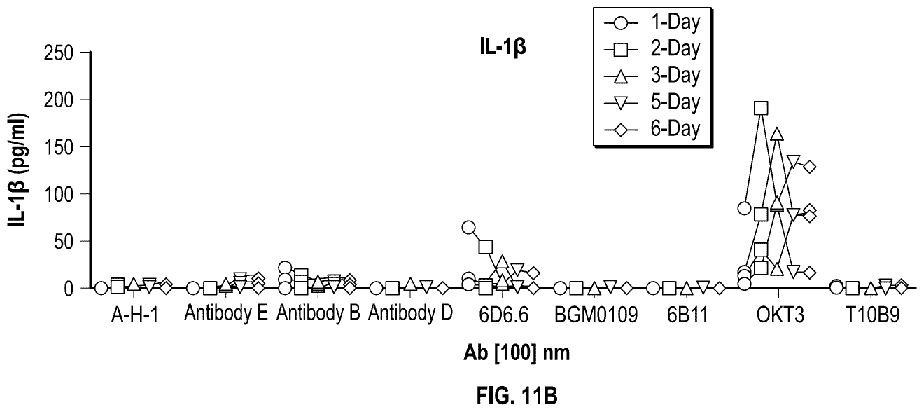

The production of cytokines IL-6, IL-1β and TNF-alpha which are associated with “cytokine storms” (and accordingly CRS) was also assessed under similar conditions. FIGS. 9A, 9A and 10A shows that while PBMCs activated with anti-CD3e antibodies demonstrate production of IL-6 (FIG. 9A), TNF-alpha (FIG. 10A) and IL-1β (FIG. 11A), no or little induction of these cytokines was observed with PBMCs activated with A-H.1 or A-H.2. As shown in FIGS. 10B and 11B, TNF-alpha and IL-1β production was not induced by activation of PBMCs with any of the anti-TCR Vβ antibodies.

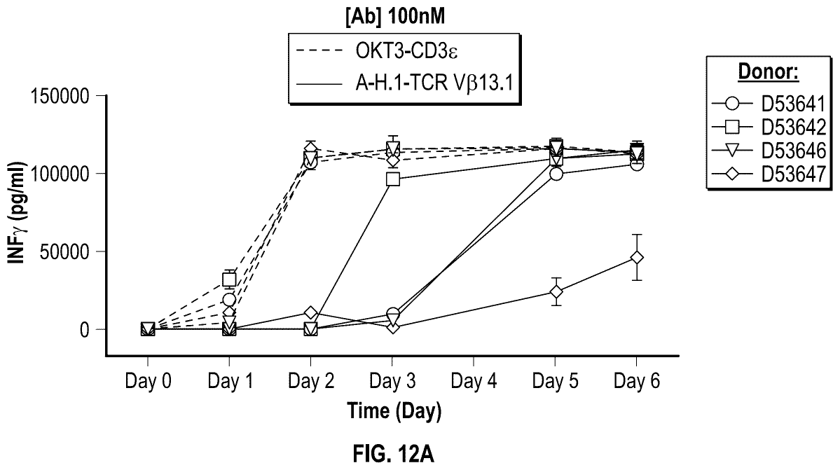

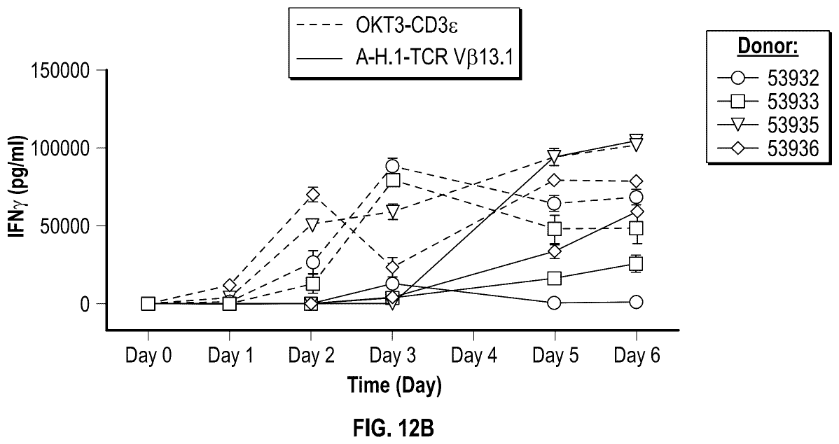

It was further noted that the kinetics of IFNg production by A-H.1-activated CD3+ T cells was delayed relative to those produced by CD3+ T cells activated by anti-CD3e mAbs (OKT3 and SP34-2) (FIGS. 12A and 12B).

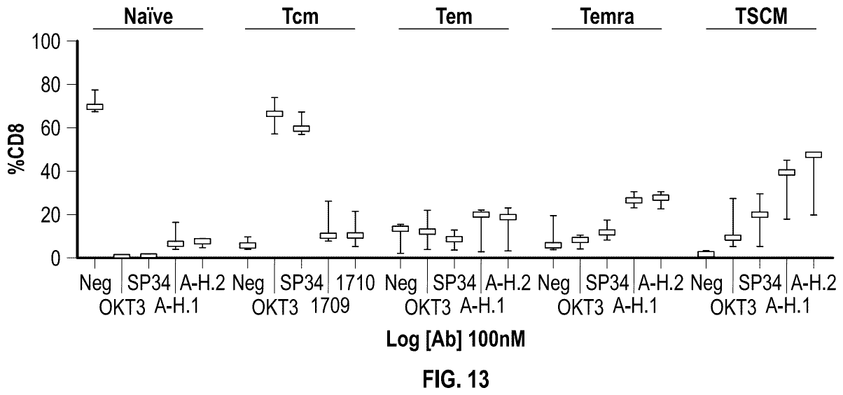

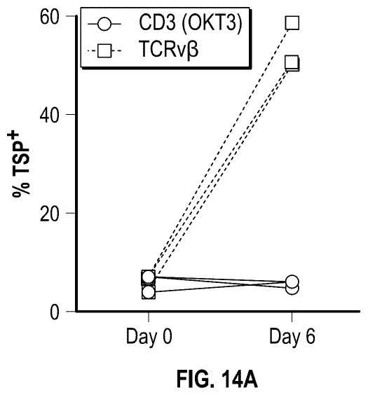

Finally, it was observed that the subset of memory effector T cells known as TEMRA Was preferentially expanded in CD8+ T cells activated by A-H.1 or A-H.2 (FIG. 13). Isolated human PBMCs were activated with immobilized (plate-coated) anti-CD3e or anti-TCR Vβ13.1 at 100 nM for 6-days. After a 6-day incubation. T-cell subsets were identified by FACS staining for surface markers for Naïve T cell (CD8+, CD95-, CD45RA+, CCR7+), T stem cell memory (TSCM; CD8+, CD95+, CD45RA+, CCR7+), T central memory (Tcm; CD8+, CD95+. CD45RA-, CCR7+). T effector memory (Tem; CD8+, CD95+, CD45RA−, CCR7-), and T effector memory re-expressing CD45RA (Temra; CD8+. CD95+, CD45RA+, CCR7−). Human PBMC's activated by anti-TCR Vβ13.1 antibodies (A-H.1 or A-H.2) increased CD8+ TSCM and Temra T cell subsets when compared to PBMCs activated by anti-CD3e antibodies (OKT3 or SP34-2). Similar expansion was observed with CD4+ T cells.

### Conclusion

The data provided in this Example show that antibodies directed against TCR Vb can, e.g., preferentially activate a subset of T cells, leading to an expansion of T_{EMRA}, which can, e.g., promote tumor cell lysis but not CRS. Thus, bispecific constructs utilizing either a Fab or scFV or a peptide directed to the TCR Vb can, e.g., be used to activate and redirect T cells to promote tumor cell lysis for cancer immunotherapy, without, e.g., the harmful side-effects of CRS associated with anti-CD3e targeting.

### Example 4: On-Target T Cell Mediated Cytotoxicity of Multiple Myeloma (MM) Cells with a Dual-Targeting Antibody Molecule Against BCMA and a T Cell Engager

This example shows on-target T cell mediated cytotoxicity of multiple myeloma (MM) cells with dual-targeting antibody molecules that recognize a T cell engager, e.g., TCRVb, on T cells and BCMA on MM cells.

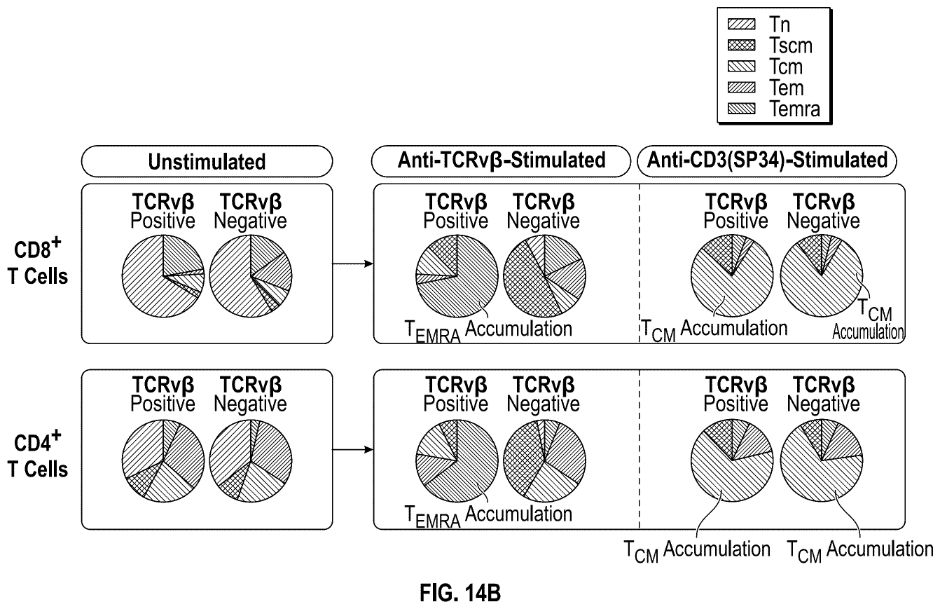

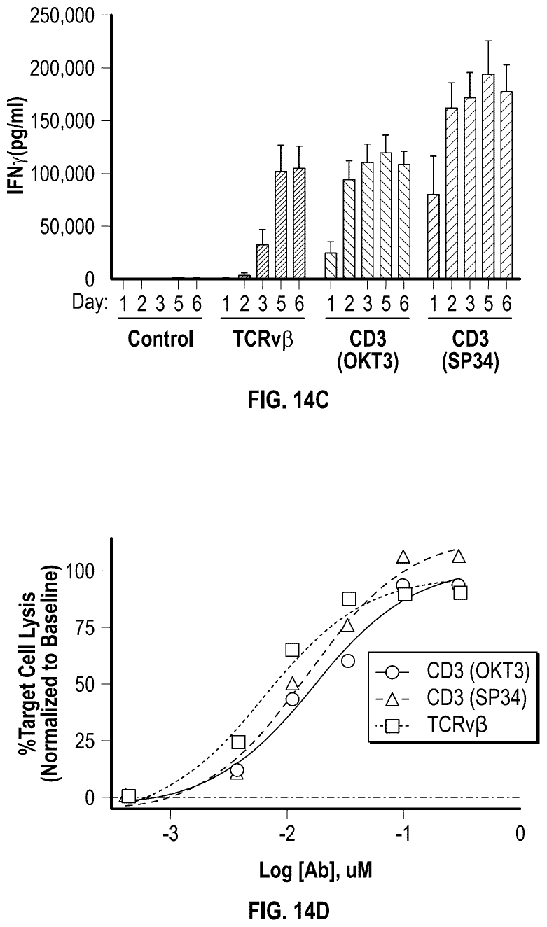

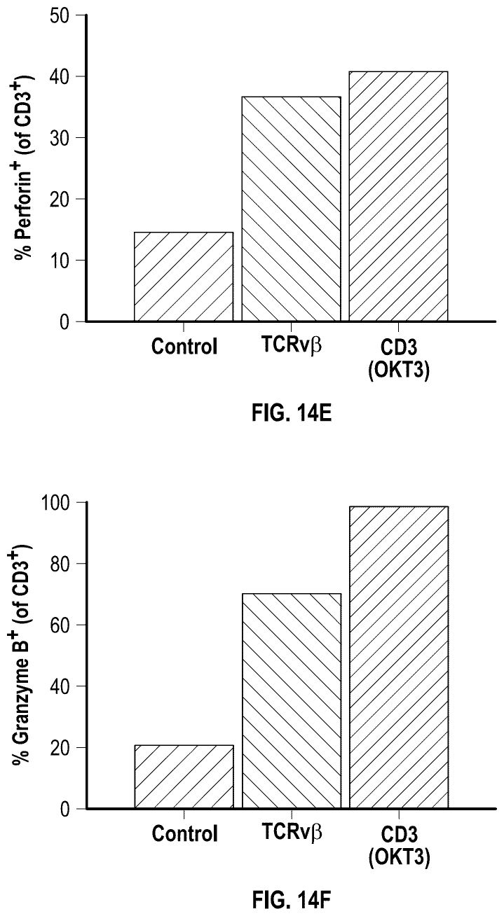

As shown in FIG. 14A, purified human T cells activated with plate-bound anti-TCRVb antibody for 5 days proliferate at a higher rate than purified human T cells activated with plate-bound anti-CD3 (OKT3) antibody. Anti-TCRVb antibody stimulation of T cells resulted in selective expansion of CD45RA+ effector memory CD8+ and CD4+ T cells (TEMRA) cells (FIG. 14B). Both CD8+ and CD4+ Temra cell populations expanded more when stimulated with an anti-TCRVb antibody, compared to unstimulated cells or cells stimulated with an anti-CD3 (SP34) antibody. Anti-TCRVb antibodies resulted in delayed secretion of IFN-g by PBMCs stimulated with an anti-TCRVb antibody compared to PBMCs stimulated with anti-CD3 antibodies (FIG. 14C). Additionally, T cells stimulated with anti-TCRVb antibody or anti-CD3 antibodies resulted in comparable lysis of multiple myeloma target cells, as shown in FIG. 14D. As shown in FIGS. 14E-14F, T cells stimulated for 5 days with 100 ng/ml plate-bound an anti-TCRVb antibody, or an anti-CD3 antibody secreted perforin and Granzyme B.

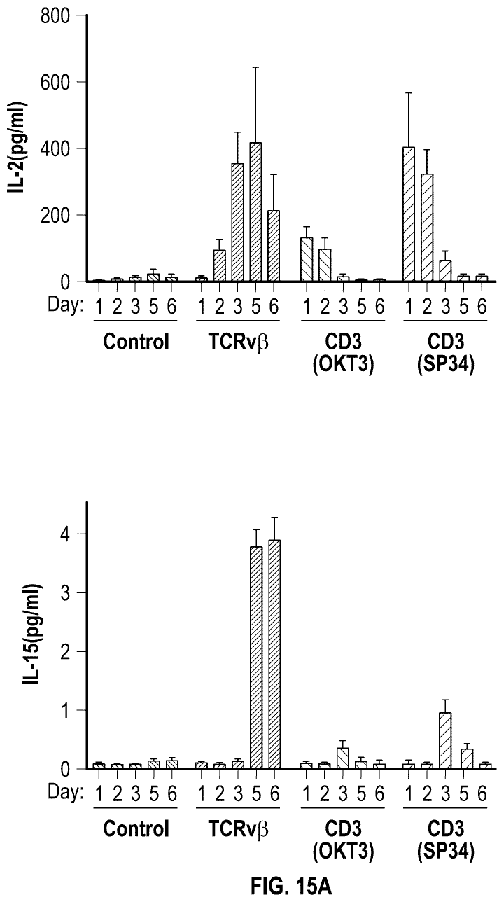

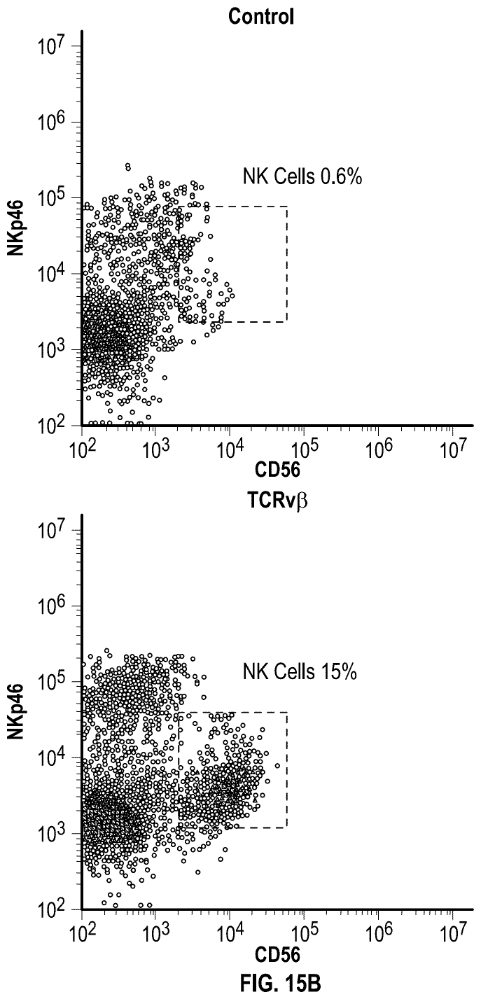

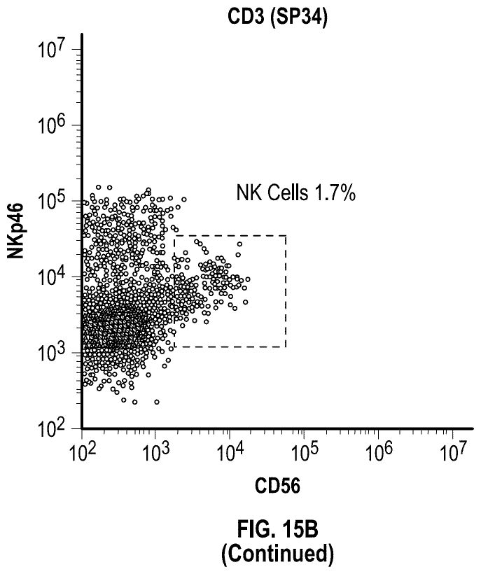

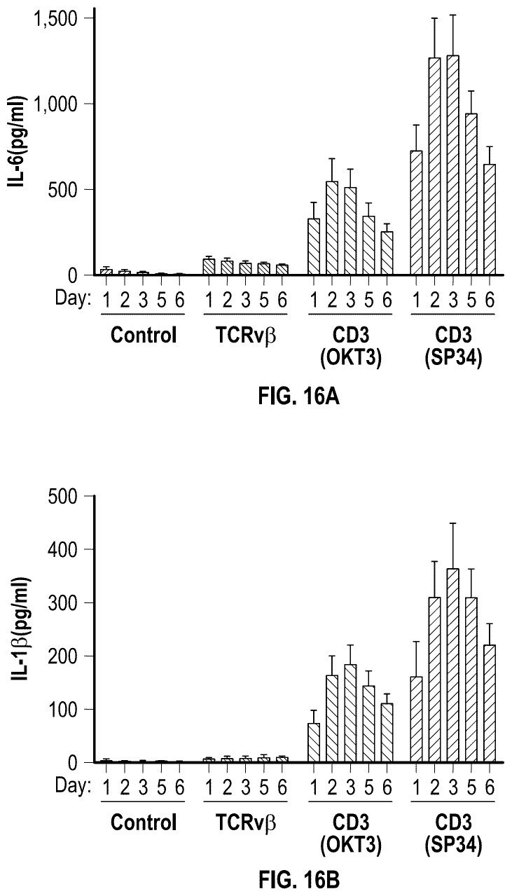

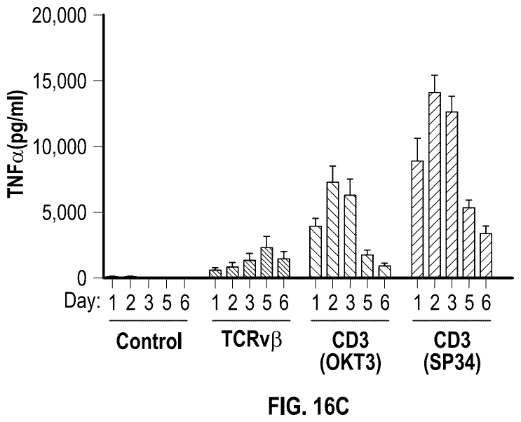

Activation of PBMCs with anti-TCRVb antibody resulted in higher production and/or secretion of IL-2 and/or IL-15 compared to PBMCs activated with an anti-OKT3 antibody (FIG. 15A). Anti-TCRVb antibody activated of PBMCs also resulted in expansion and/or survival, e.g., proliferation of Natural Killer (NK) cells (FIG. 15B). In comparison, PBMCS activated with an anti-OKT3 antibody did not result in NK cell expansion. Further, as described in Example 3, PBMCs activated with an anti-TCRVb antibody did not result in the production of cytokines IL-6, IL-1β and TNF-alpha which are associated with CRS (FIG. 16). These in vitro characterization studies show that in some embodiments, anti-TCRVb antibodies, e.g., activate and/or stimulate, T cells to promote T cell killing as evidenced by target cell lysis, perforin secretion and granzyme B secretion, and secretion of IFN-g with, e.g., delayed kinetics.

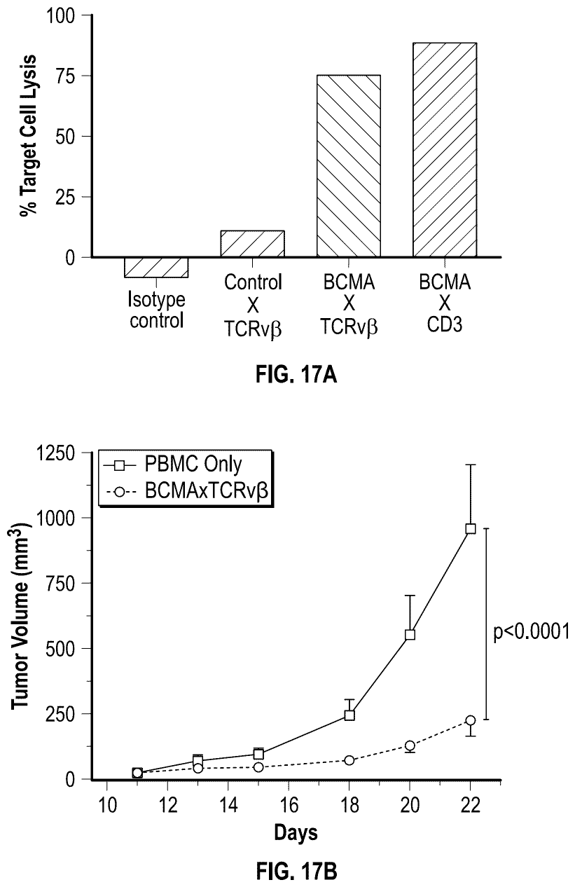

Next, the ability of a dual-targeting antibody molecule (Molecule I), which targets BCMA on one arm and TCRVb on the other arm, to target and kill multiple myeloma (MM) cells was tested. Healthy donor PBMCs were co-incubated with the RMPI8226 MM cell line and one of the following dual-targeting antibody molecules: BCMA-TCRVb (Molecule I), BCMA-CD3, or Control-TCRVb; or an isotype control Target cell lysis was then assessed using flow cytometry. As shown in FIG. 17A, the dual-targeting BCMA-TCRVb antibody molecule (Molecule I) resulted in killing of MM cells in vitro.

The dual-targeting BCMA-TCRVb antibody molecule (Molecule 1) was further tested in vivo for its ability to inhibit MM tumor growth in a MM mouse model. The NCI-H929 cell line was injected in NOD-scid IL2rγnull (NSG) recipient mice on Day 0 followed by delivery of PBMCs on Day 9. On Days 12, 15, 18 and 21, the dual-targeting BCMA-TCRVb antibody molecule (Molecule I) was administered via intraperitoneal injection at a dose of 0.5 mg/kg. FIG. 17B shows prevention, e.g., inhibition, of MM tumor growth in vivo with the dual-targeting BCMA-TCRVb antibody molecule (Molecule I). These results demonstrate that in some embodiments the dual-targeting BCMA-TCRVb antibody molecule, e.g., can kill tumor cells, e.g., MM tumor cells, in vitro and in vivo. Accordingly, in some embodiments, a dual-targeting BCMA-TCRVb antibody molecule can be used, e.g., as a therapy for cancer, e.g., a hematological cancer, e.g., MM.

### Example 5: In Vitro Cytotoxicity of a Dual-Targeting Antibody Molecule Against FcRH5 and a T Cell Engager

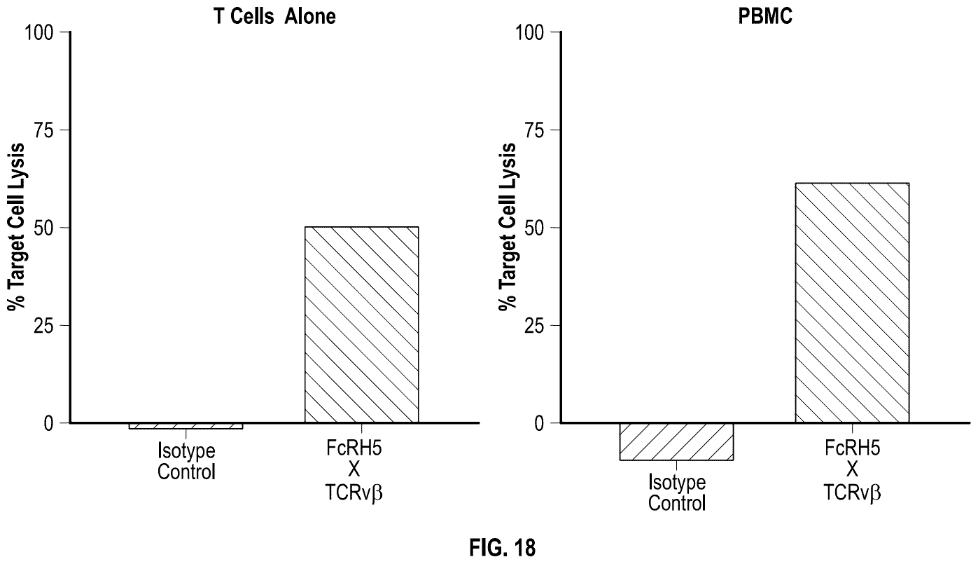

This example shows in vitro cytotoxicity on multiple myeloma (MM) cells with a dual-targeting antibody molecule that recognizes a T cell engager, e.g., TCRVb, on T cells and FcRH5 on MM cells. Healthy donor PBMCs or purified T cells were co-incubated with the MOL8M MM cell line and a dual-targeting antibody molecule which targets FcRH5 on one arm and TCRVb on the other arm (Molecule E), or with an isotype control antibody. Target cell lysis was then assessed using flow cytometry. As shown in FIG. 18, the dual targeting FcRH5-TCRVb molecule (Molecule E) resulted in killing of MM cells by both purified T cells or PBMCs. This shows that the dual targeting FcRH5-TCRVb molecule can target and promote killing of MM cells by immune cells, e.g., in PBMCs, including T cells.

### Example 6: Characteristics of Anti-TCR Vβ8a Antibodies

This Example shows in vitro characterization of anti-TCR Vβ8a antibodies (B-H.1). TCR Vβ8 is also referred to as TCR Vβ12 (as described in Table 8A). Isolated human PBMCs were activated with immobilized (plate-coated) anti-CD3ε or anti-TCR Vβ8a at 100 nM, and cell culture supernatants were collected on day 1, 2, 3, 5, 6 and 8 post stimulation. Cytokines (IFNγ, IL-2, TNFα, IL-1β or IL-6) were measured using MSD technology platform (MesoScale Discovery) as described in the manufacturer's protocol.

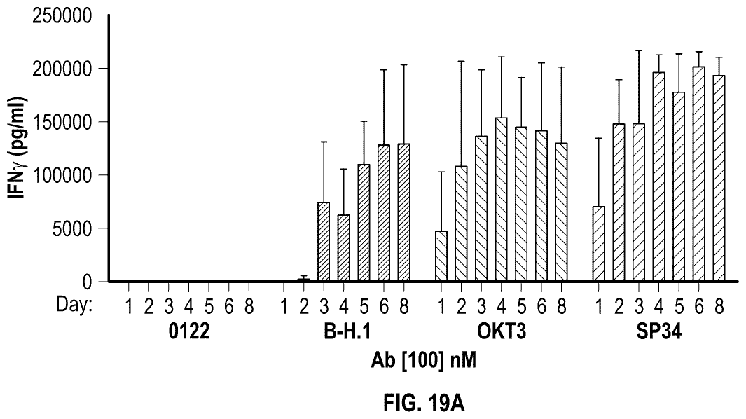

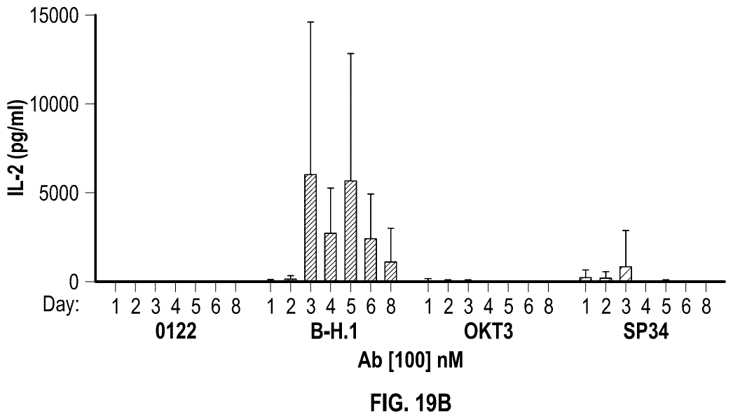

As shown in FIGS. 19A-19B, Human PBMCs activated by anti-TCR Vβ8a antibodies (B-H.1) produce similar or reduced levels of IFNγ (FIG. 19A) and higher levels IL-2 (FIG. 19B) when compared to those activated by anti-CD3e antibodies (OKT3 or SP34-2).

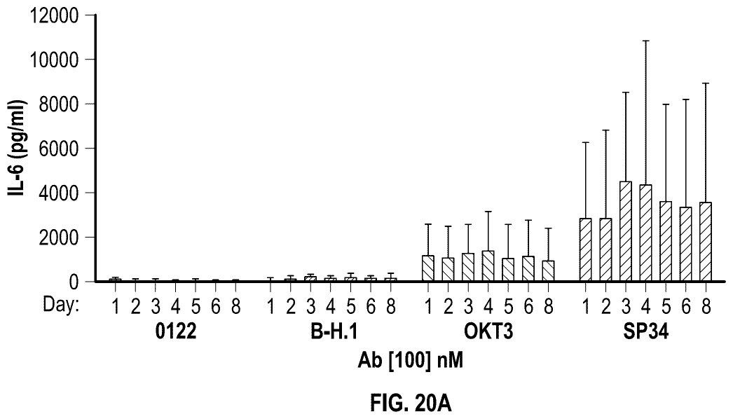

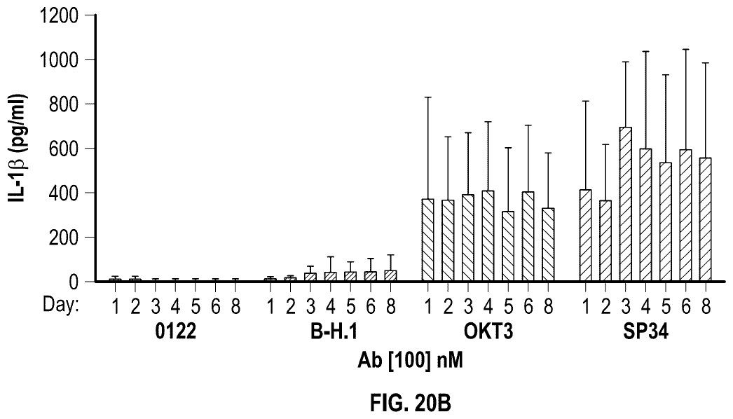

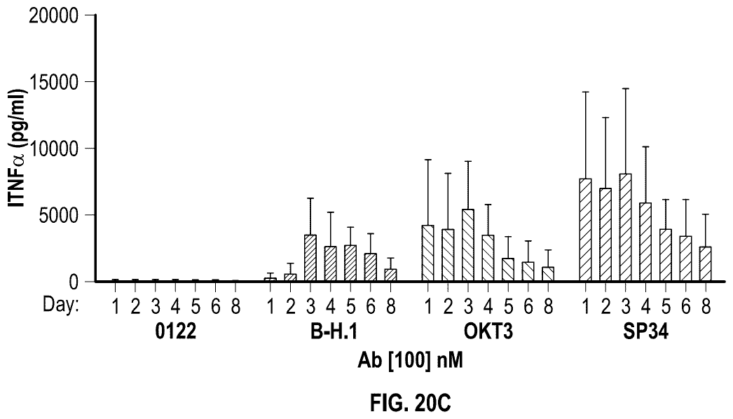

FIGS. 20A-20B show that human PBMCs activated by anti-TCR Vβ8a antibodies (B-H.1) do not produce significant levels of IL-6, or IL1b. Activation of human PBMCs with anti-TCR Vβ8a antibodies (B-H. 1) also results in lesser TNFalpha when compared to PBMCs activated by anti-CD3€ antibodies (OKT3 or SP34-2) (see FIG. 20C).

In summary, as shown in Example 3, this Example shows that anti-TCR Vβ8a antibodies can, e.g., preferentially induce expression of T cell cytokines, e.g., IL-2 and IFNg, but not production of cytokines IL-6. IL-1β and TNF-alpba which are associated with “cytokine storms” (and accordingly CRS).

### Example 7: Characteristics of Anti-TCRβV Antibody D Antibody

This Example describes characterization of anti-TCRβV antibodies which can bind and activate a subset of T cells, but with, e.g., no or markedly reduced, CRS.

Human PBMCs were isolated from whole blood followed by solid-phase (plate-coated) stimulation with anti-TCR Vβ12 antibody (Antibody D) or anti-CD3e antibodies (OKT3) at 100 nM. Supernatant was collected on Days 1, 2, 3, 5, or 6 followed by multiplex cytokine analysis for IFNg, IL-2, IL-6, IL-1beta, or TNFalpha. The data was quantified using MSD (Meso Scale Discovery) platform, following the manufacturer's protocol.

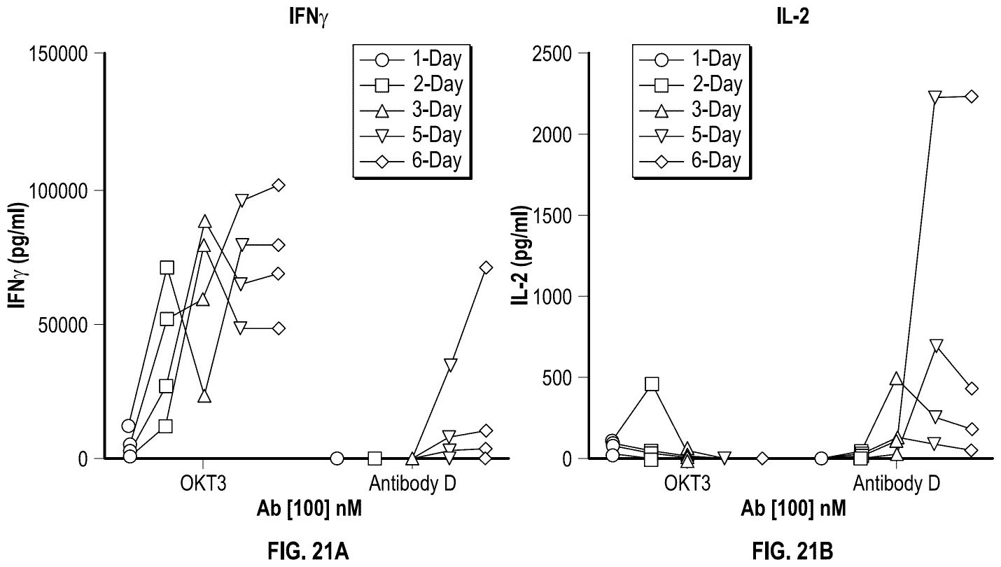

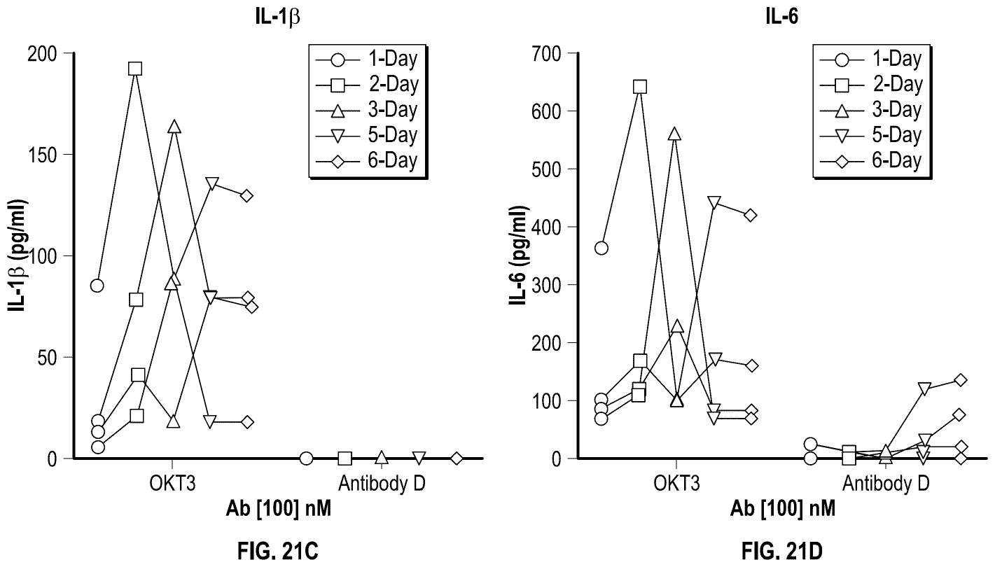

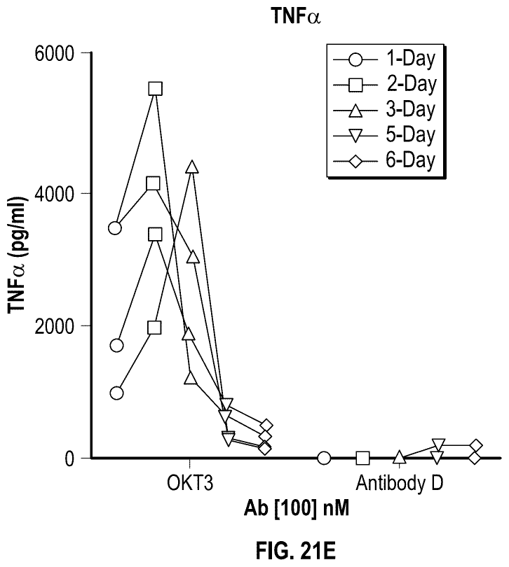

As shown in FIG. 21A, when plate-bound anti-TCR Vβ12 antibody (Antibody D) or anti-CD3e antibodies (OKT3) were used to activate human PBMCs, the T cell cytokine IFNg was induced. With respect to IL-2 production, PBMCs activated with anti-TCR Vβ12 antibody (Antibody D) resulted in increased IL-2 production with delayed kinetics (FIG. 21B) as compared to PBMCs activated with anti-CD3e antibodies (OKT3).

The production of cytokines IL-6, IL-1β and TNF-alpha which are associated with “cytokine storms” (and accordingly CRS) was also assessed under similar conditions. FIGS. 21C-21E show that that while PBMCs activated with anti-CD3e antibodies demonstrate production of IL-6 (FIG. 21D). TNF-alpha (FIG. 21C) and IL-1β (FIG. 21E), no or little induction of these cytokines was observed with PBMCs activated with anti-TCR Vβ12 antibody (Antibody D).

The data provided in this Example show that antibodies directed against TCR Vβ can, e.g., preferentially activate a subset of T cells, and do not result in induction of cytokines associated with cytokine storms or CRS.

### Example 8: Characteristics of Anti-TCRβV Antibody E

This Example describes characterization of anti-TCRβV antibodies which can bind and activate a subset of T cells, but with, e.g., no or markedly reduced, CRS.

Human PBMCs were isolated from whole blood followed by solid-phase (plate-coated) stimulation with anti-TCR Vβ5 antibody (Antibody E) or anti-CD3e antibodies (OKT3 and SP34-2), each at 100 nM. Supernatant was collected on Days 1, 3, 5, or 7 followed by multiplex cytokine analysis for IFNg, IL-2, IL-6, IL-1beta, IL-10 or TNFalpha. The data was quantified using MSD (Meso Scale Discovery) platform, following the manufacturer's protocol.

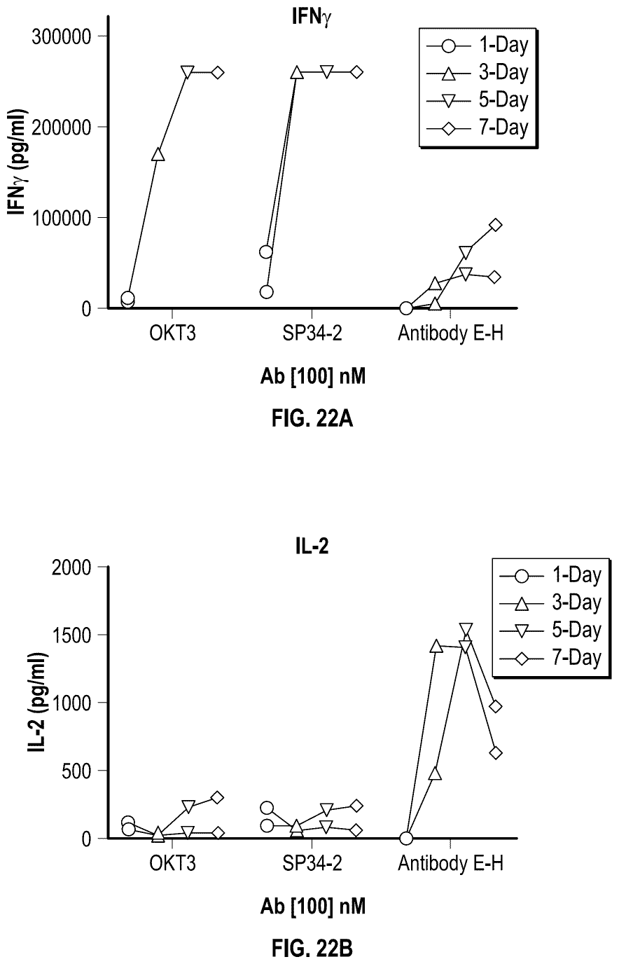

As shown in FIG. 22A, when plate-bound anti-TCR Vβ5 antibody (Antibody E) or anti-CD3e antibodies (OKT3 and SP34-2) were used to activate human PBMCs, the T cell cytokine IFNg was induced. With respect to IL-2 production. PBMCs activated with anti-TCR Vβ5 antibody (Antibody E) resulted in increased IL-2 production with delayed kinetics (FIG. 22B) as compared to PBMCs activated with anti-CD3e antibodies (OKT3 or SP34-2).

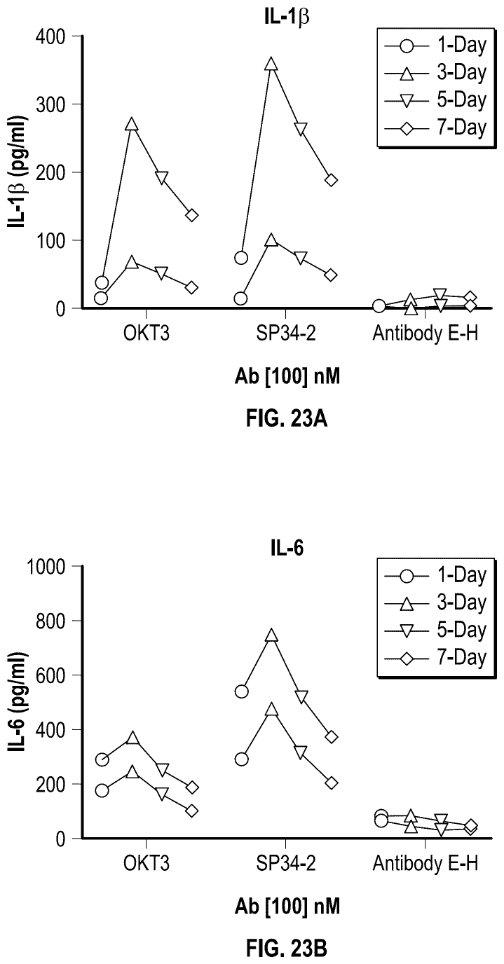

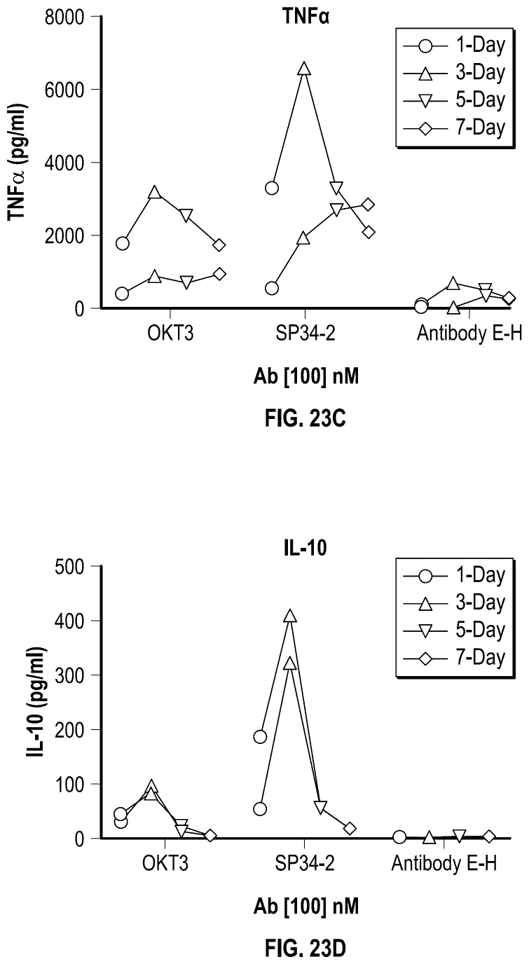

The production of cytokines IL-6, IL-1β, IL-10 and TNF-alpha which are associated with “cytokine storms” (and accordingly CRS) was also assessed under similar conditions. FIGS. 23A-23D show that that while PBMCs activated with anti-CD3e antibodies demonstrate production of IL-1β (FIG. 23A), IL-6 (FIG. 23B), TNF-alpha (FIG. 23C) and IL-10 (FIG. 23D), no or little induction of these cytokines was observed with PBMCs activated with anti-TCR Vβ5 antibody (Antibody E).

The data provided in this Example show that antibodies directed against TCR Vβ can, e.g., preferentially activate a subset of T cells, and do not result in induction of cytokines associated with cytokine storms or CRS

### Example 9: Characteristics of a Dual-Targeting Antibody Molecule Against BCMA and TCRβV

This Example describes characterization of a dual targeting antibody (e.g., a bispecific molecule) comprising an anti-TCRβV binding moiety and a BCMA binding moiety (Molecule H) which can bind and activate a subset of T cells, but with, e.g., no or markedly reduced, CRS.

Human PBMCs were isolated from whole blood followed by solid-phase (plate-coated) stimulation with an anti-TCRβV×BCMA bispecific molecule (Molecule H) or anti-CD3e antibodies (OKT3), each at 100 nM. Supernatant was collected on Days 1, 2, 3, or 5 followed by multiplex cytokine analysis for IFNg, IL-2, IL-6, IL-1beta, IL-10 or TNFalpha. The data was quantified using MSD (Meso Scale Discovery) platform, following the manufacturer's protocol.

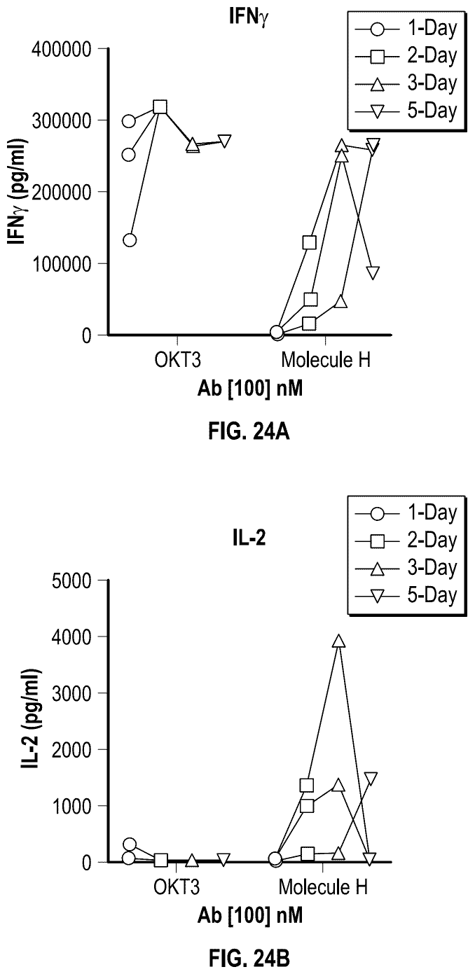

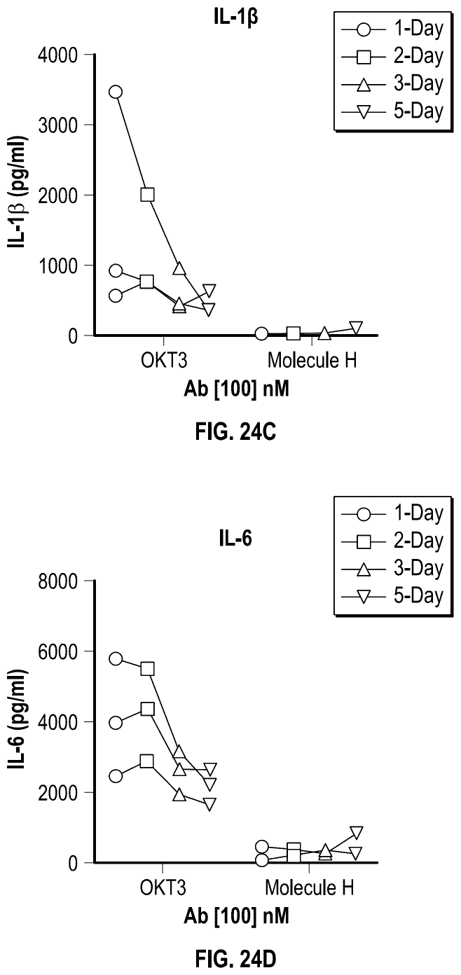

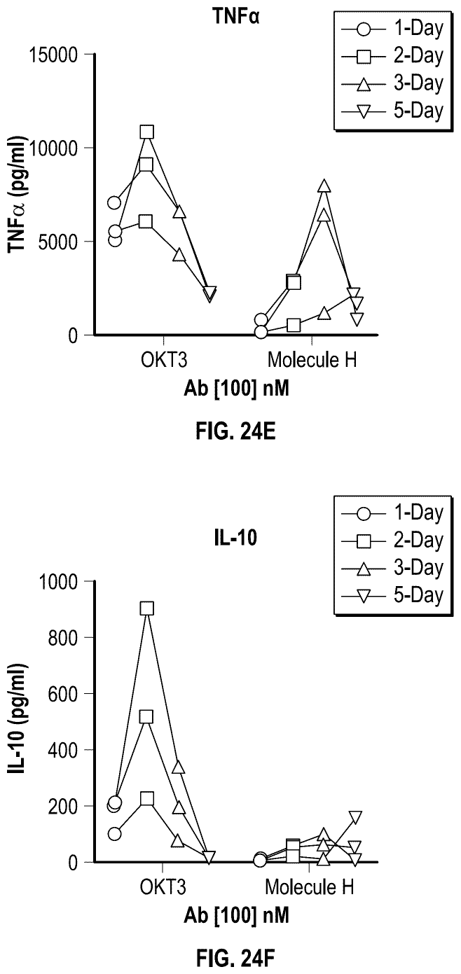

As shown in FIG. 24A, when plate-bound anti-TCRβV×BCMA bispecific molecule (Molecule H) or anti-CD3e antibodies (OKT3) were used to activate human PBMCs, the T cell cytokine IFNg was induced. With respect to IL-2 production, PBMCs activated with anti-TCRβV×BCMA bispecific molecule (Molecule H) resulted in increased IL-2 production (FIG. 24B) as compared to PBMCs activated with anti-CD3e antibodies (OKT3).

The production of cytokines IL-6, IL-1β, IL-10 and TNF-alpha which are associated with “cytokine storms” (and accordingly CRS) was also assessed under similar conditions. FIGS. 24C-24E show that that while PBMCs activated with anti-CD3e antibodies demonstrate production of IL-1β (FIG. 24C), IL-6 (FIG. 24D), TNF-alpha (FIG. 24D) and IL-10 (FIG. 24E), no or little induction of these cytokines was observed with PBMCs activated with anti-TCRβV×BCMA bispecific molecule (Molecule H).

The data provided in this Example show that antibodies directed against TCR Vβ can, e.g., preferentially activate a subset of T cells, and do not result in induction of cytokines associated with cytokine storm or CRS.

### Example 10: Cytokine and Chemokine Profile of Anti-TCRVb Antibodies

This Examples describes cytokines and chemokines secreted by PBMCs following activation by anti-TCR Vβ antibodies.

Human PBMCs were isolated from whole blood followed by solid-phase (plate-coated) stimulation with an anti-TCRβV antibodies (A-H.1, B-H.1), or a bispecific molecule comprising an anti-TCRVb antibody (Molecule H), an isotype control (BGM0122) or an anti-CD3e antibody (SP34), each at 100 nM. Supernatant was collected on Days 1, 2, 3, 4, 5, 6, 7 and 8 followed by multiplex analysis for the indicated cytokines or chemokines. The data was quantified using MSD (Meso Scale Discovery) platform, following the manufacturer's protocol. BGM0122 comprises the amino acid sequence of

(SEQ ID NO: 3283)
METDTLLLWVLLLWVPGSTGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGKGGGGSGGGGSGLNDIFEAQ
KIEWHE

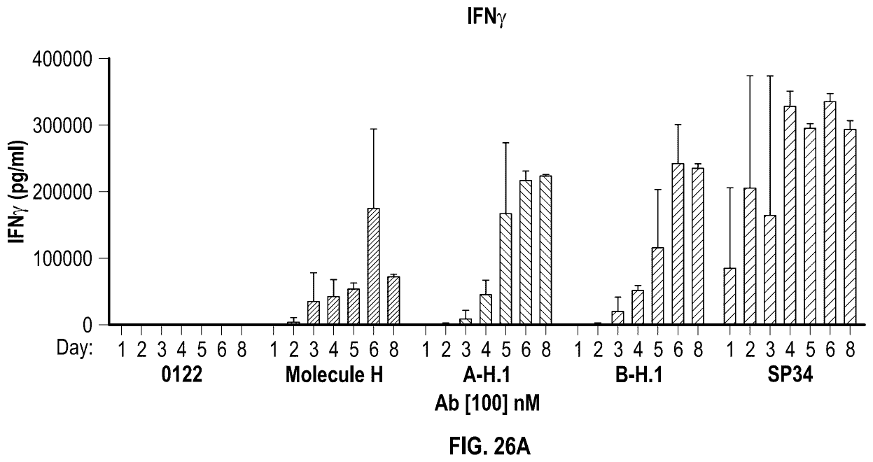

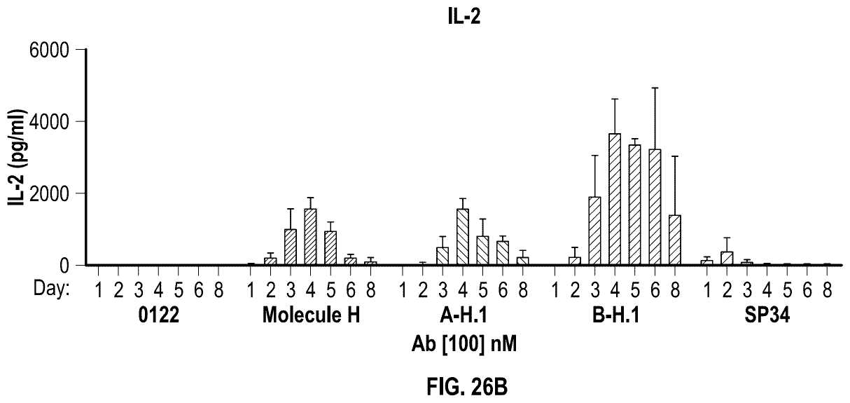

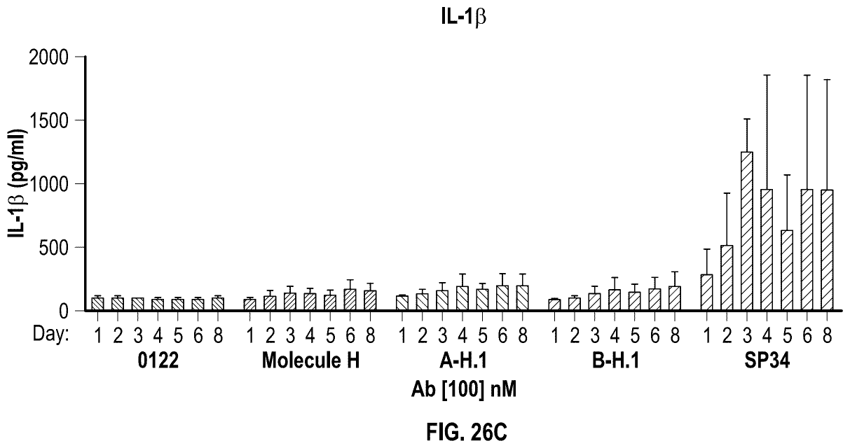

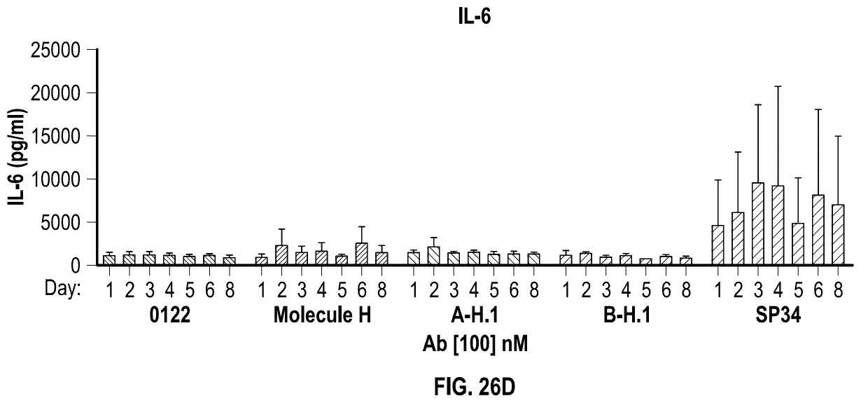

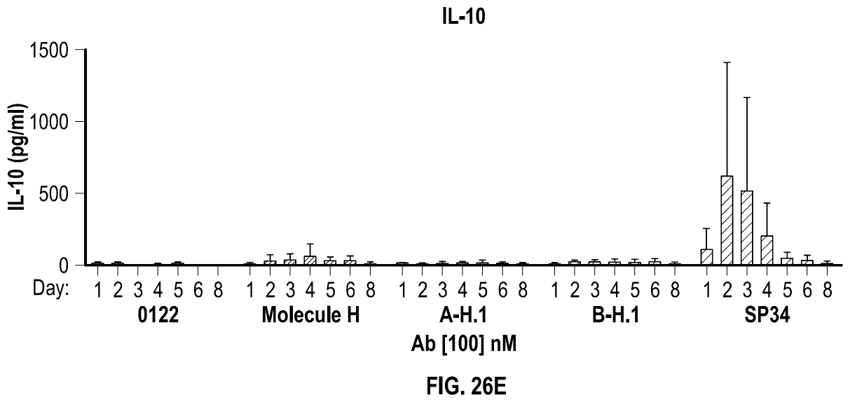

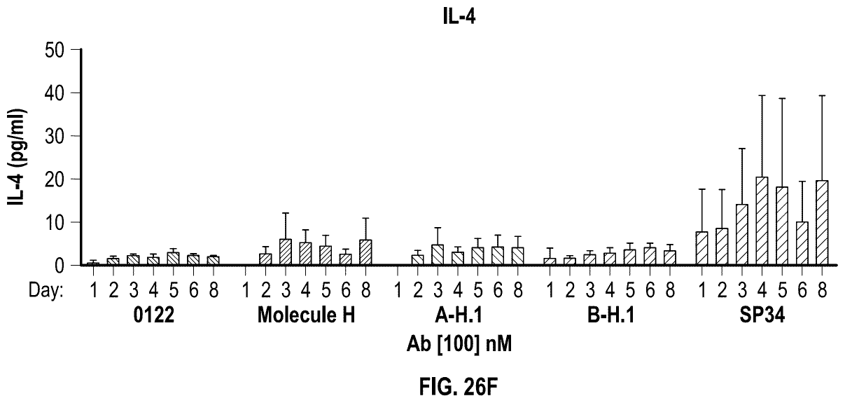

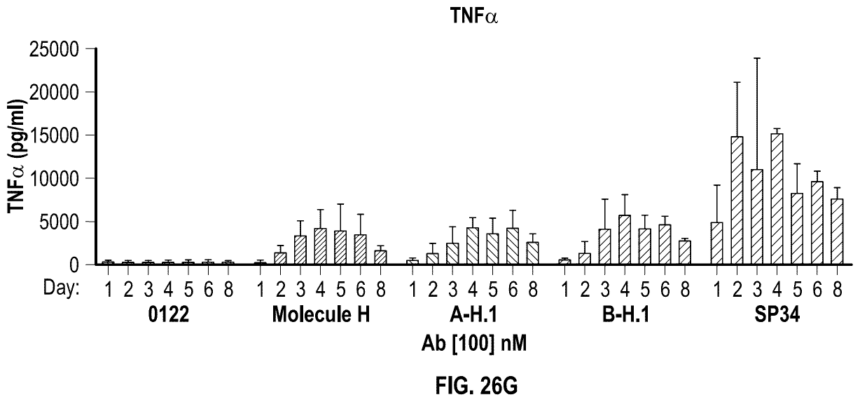

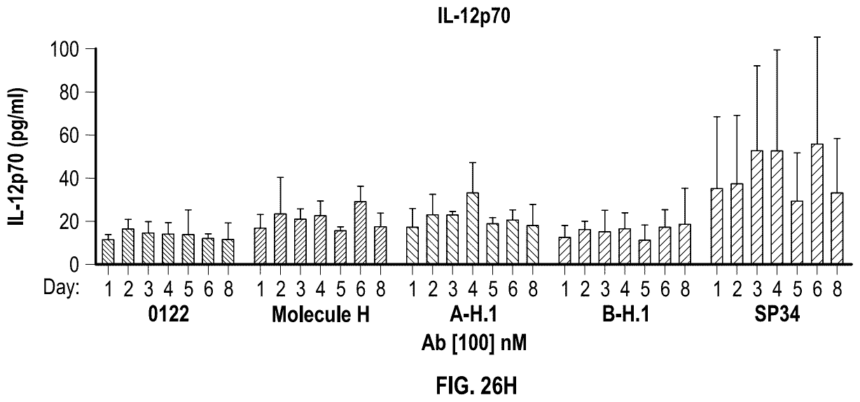

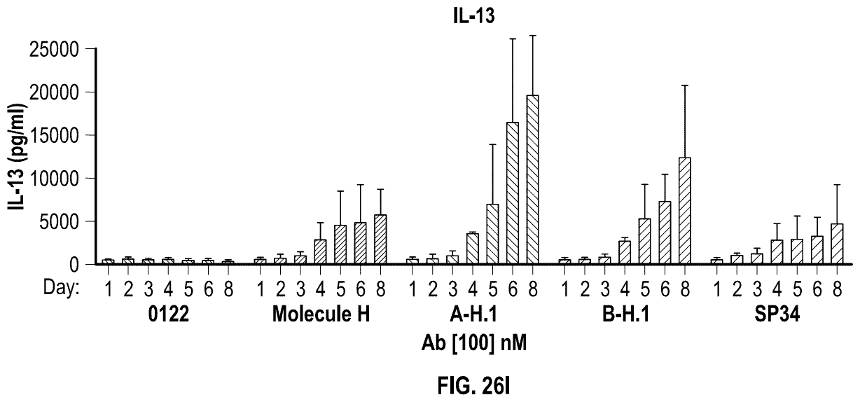

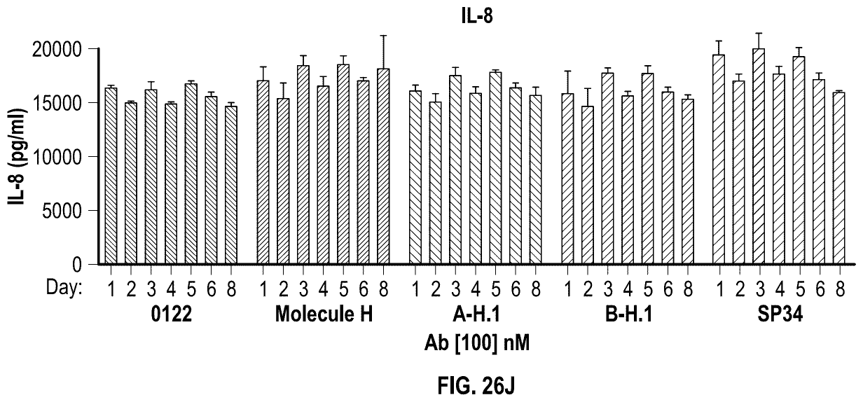

FIGS. 26A-26J, FIGS. 27A-27H, and FIGS. 28A-28L show the levels of cytokine and chemokine from PBMCs activated with the indicated antibodies.

As shown in FIG. 26A, when plate-bound anti-TCR Vβ antibodies or anti-CD3e antibodies (OKT3) were used to activate human PBMCs, the T cell cytokine IFNg was induced. With respect to IL-2 production, PBMCs activated with anti-TCR Vβ antibodies resulted in increased IL-2 production with delayed kinetics (FIG. 26B) as compared to PBMCs activated with anti-CD3e antibody (OKT3).

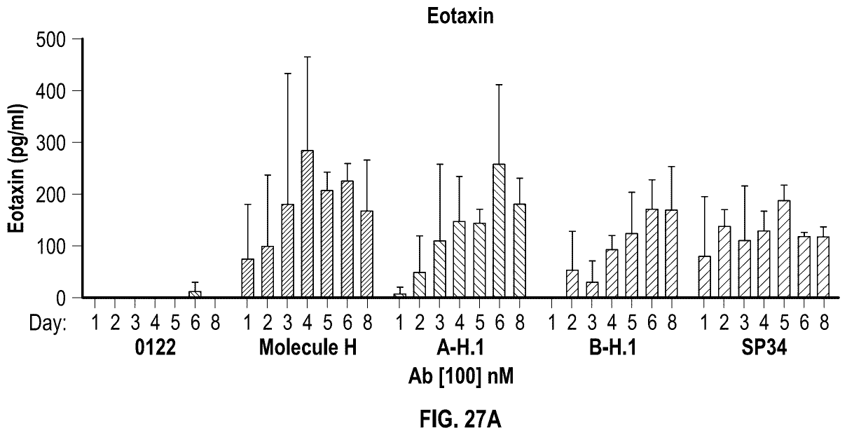

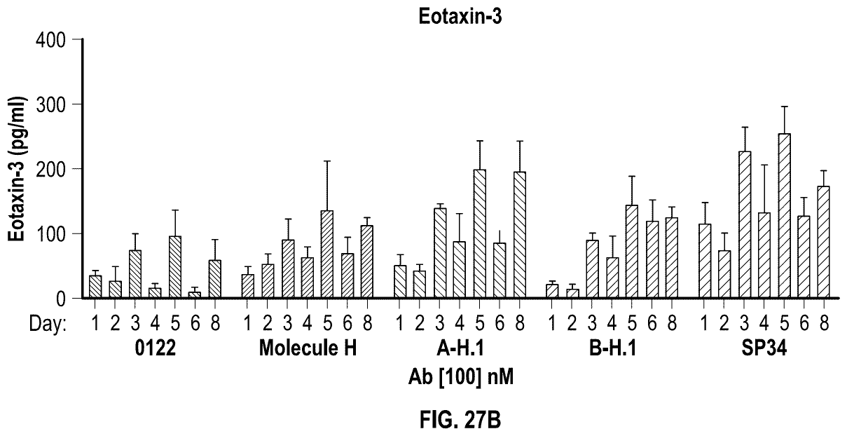

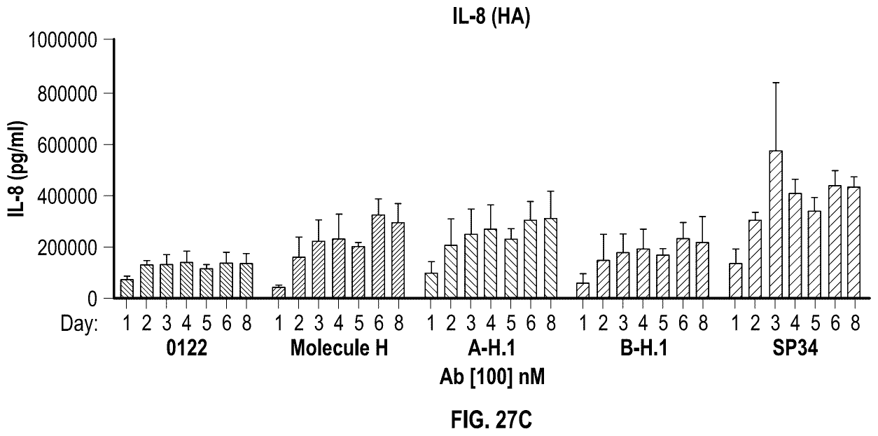

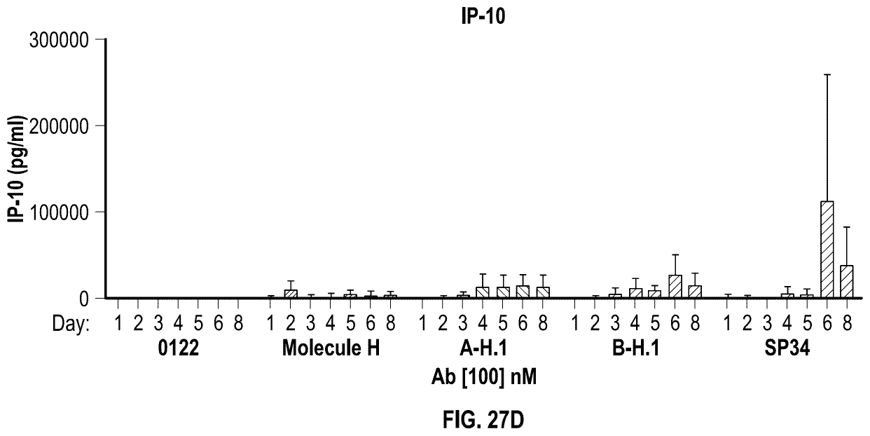

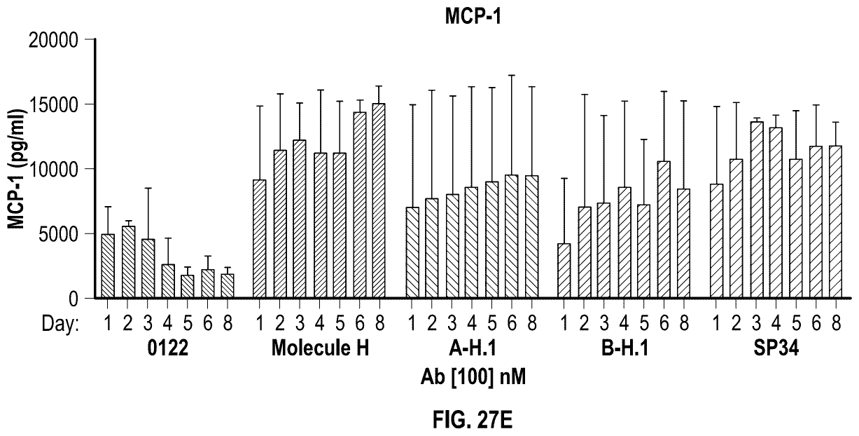

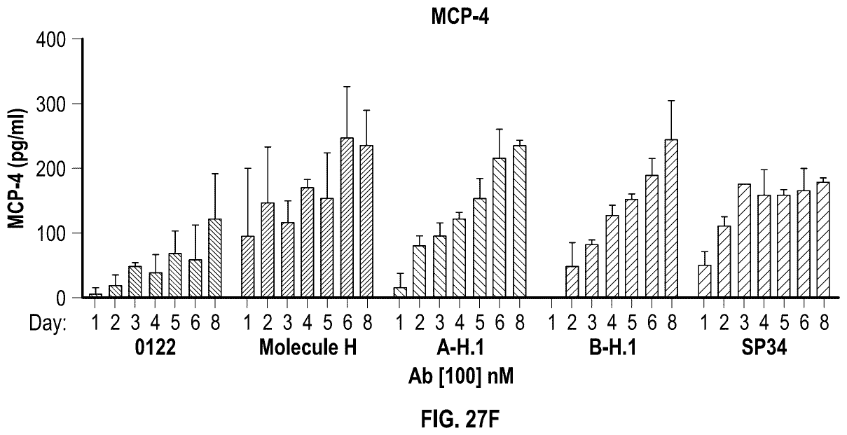

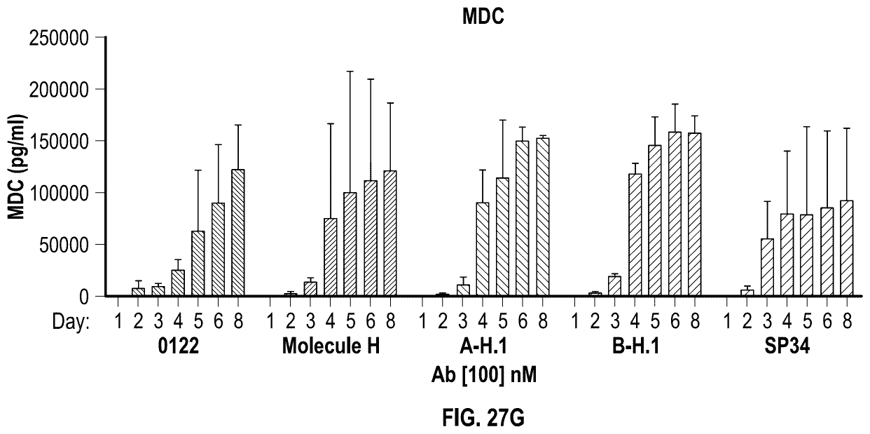

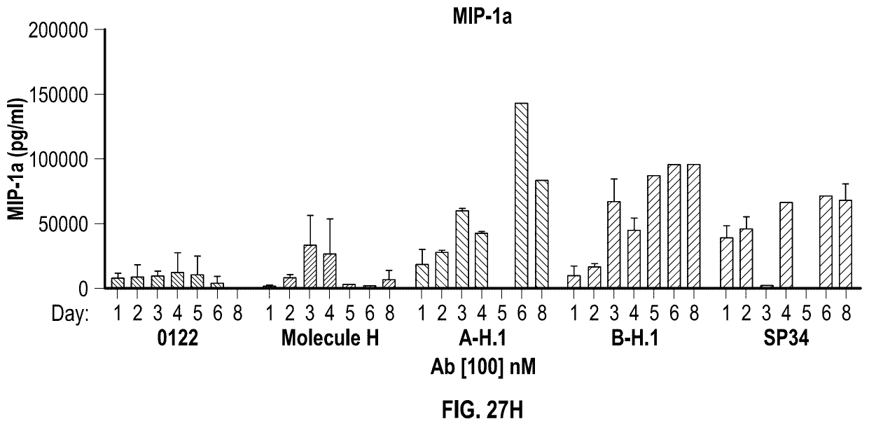

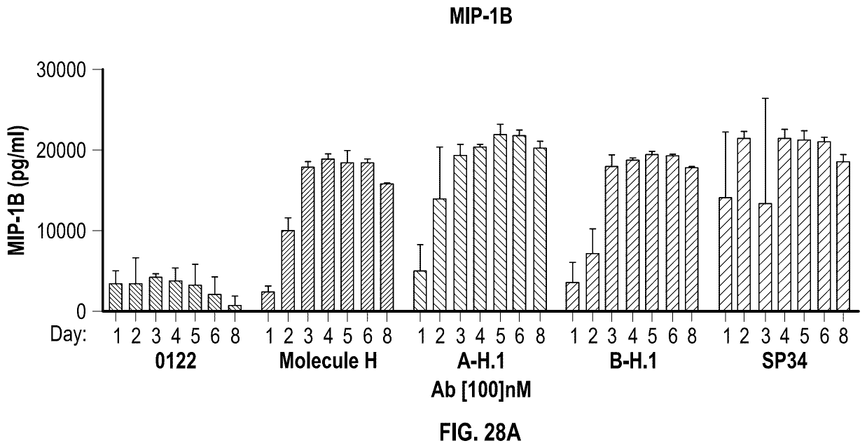

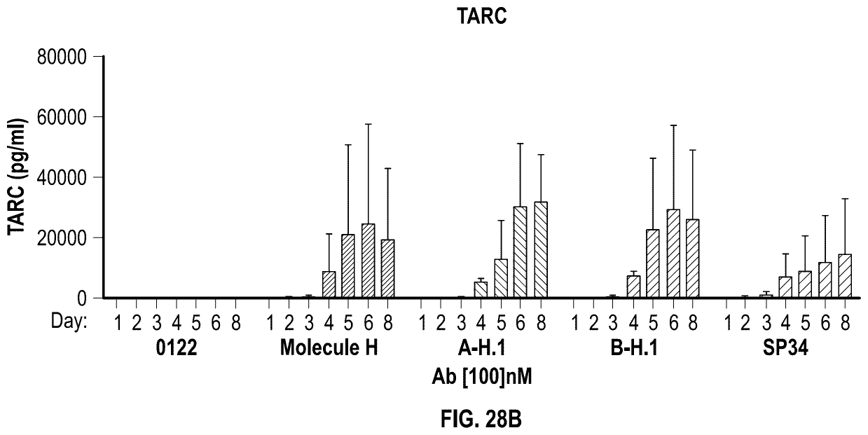

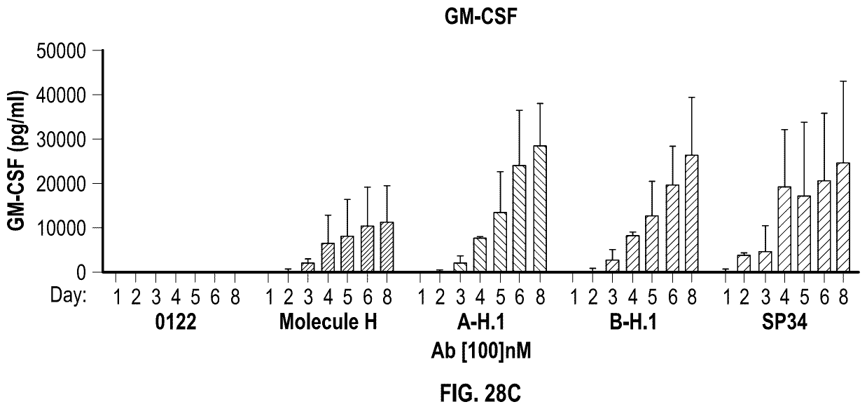

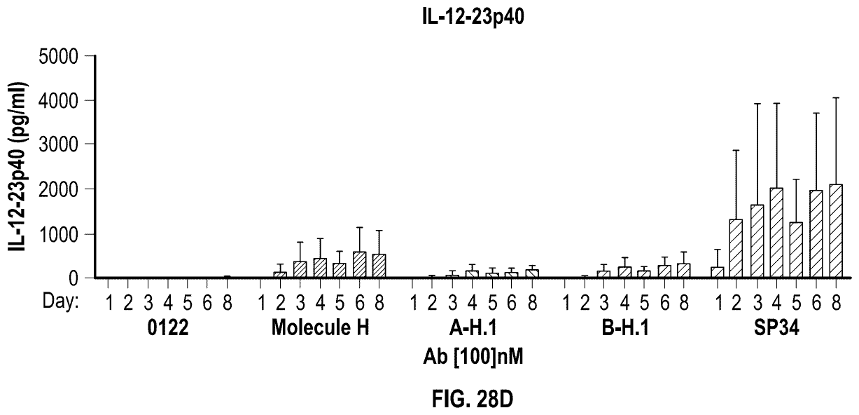

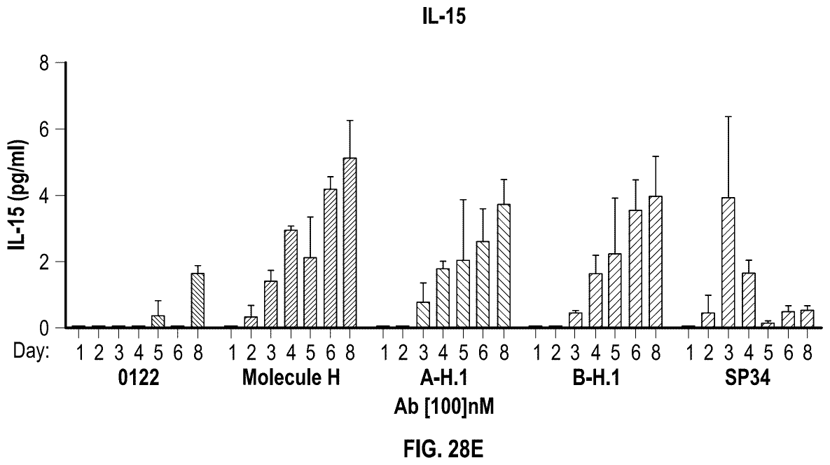

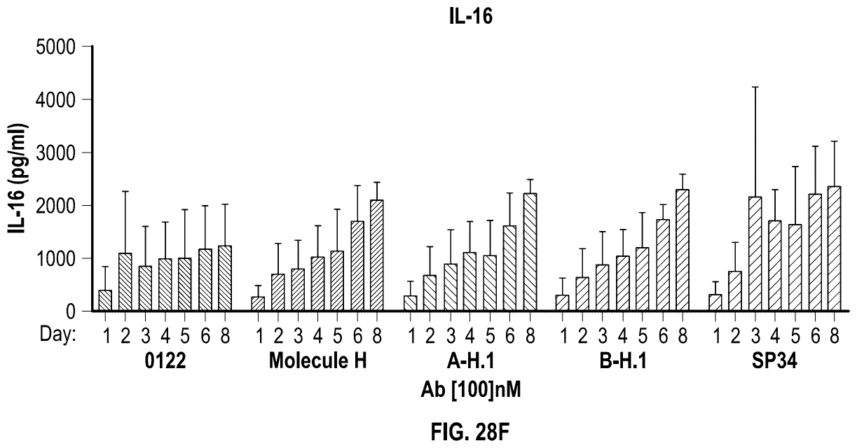

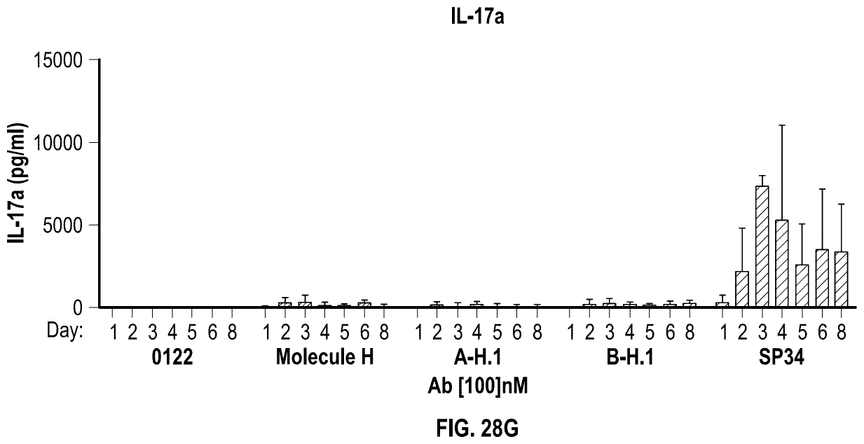

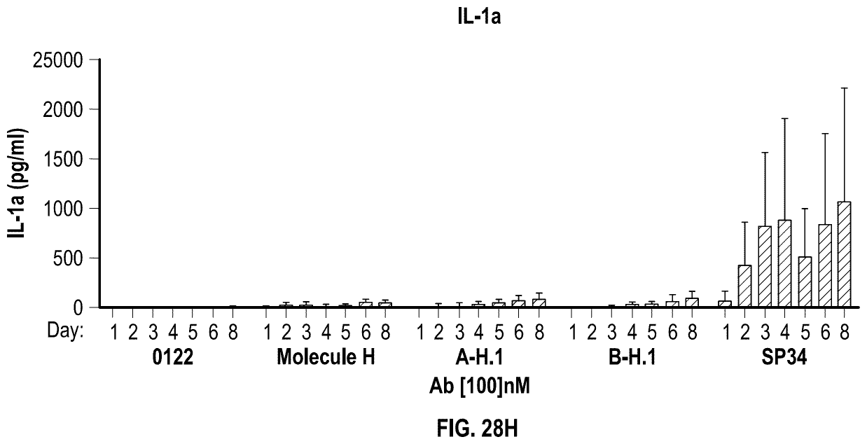

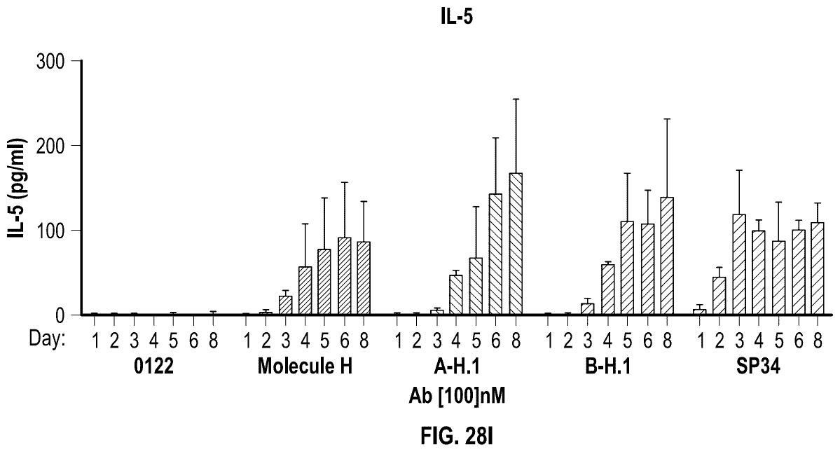

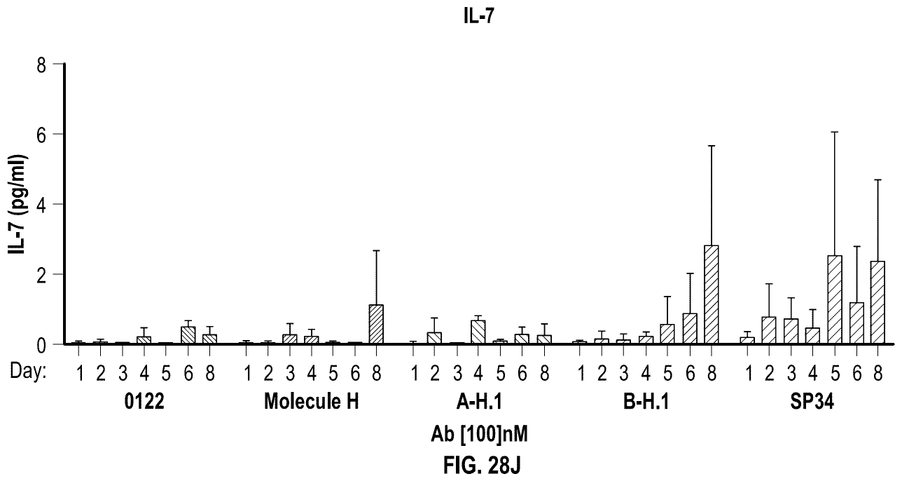

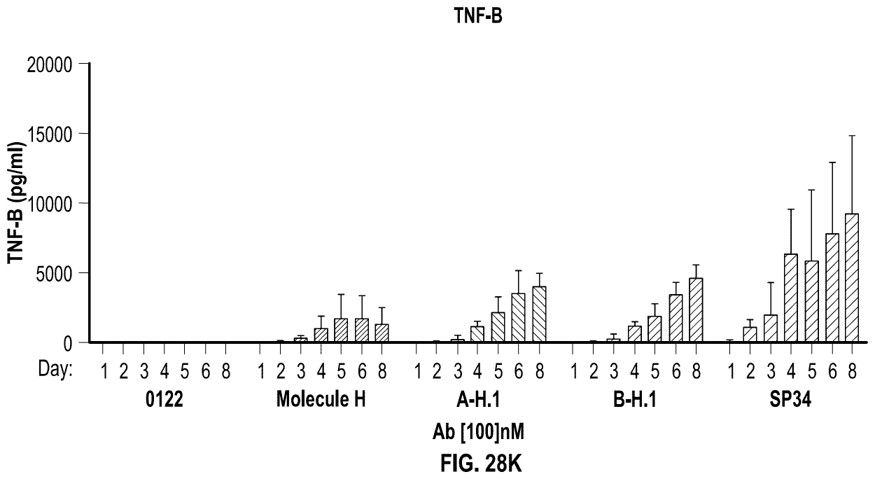

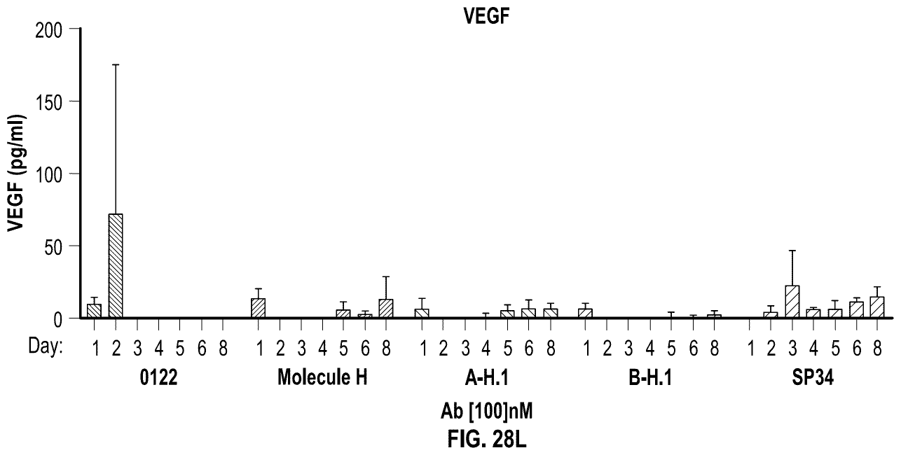

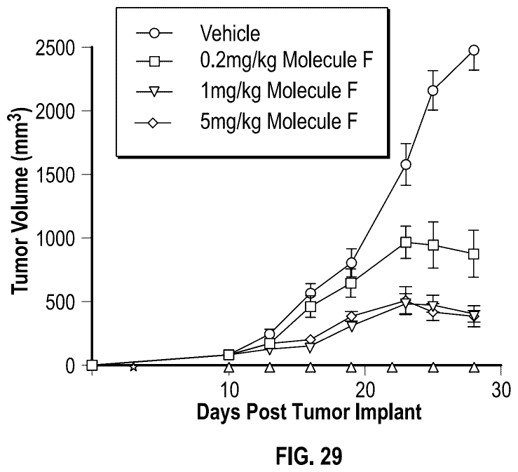

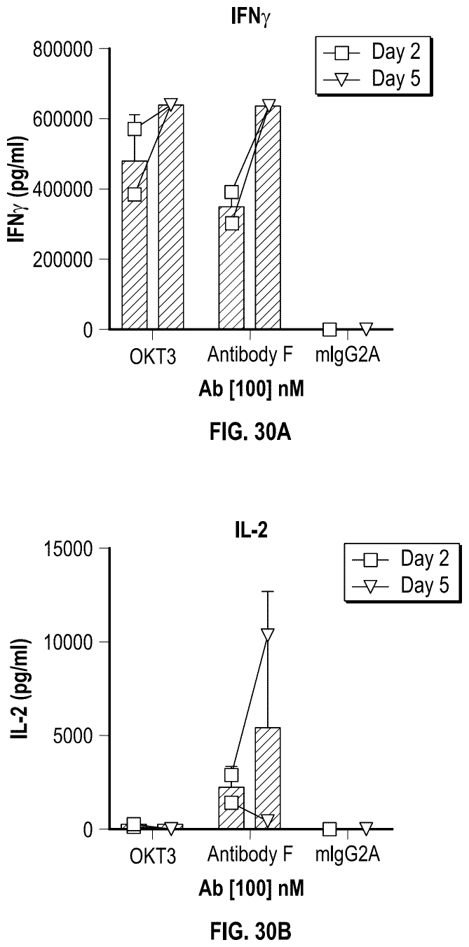

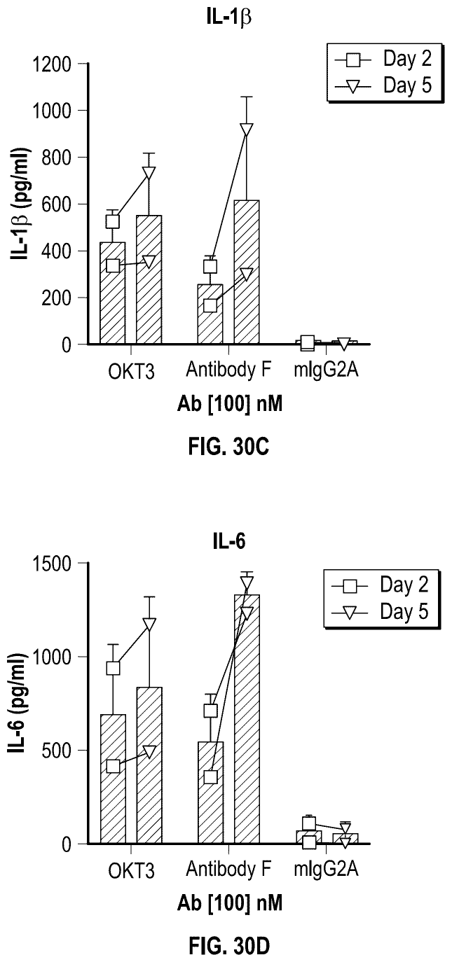

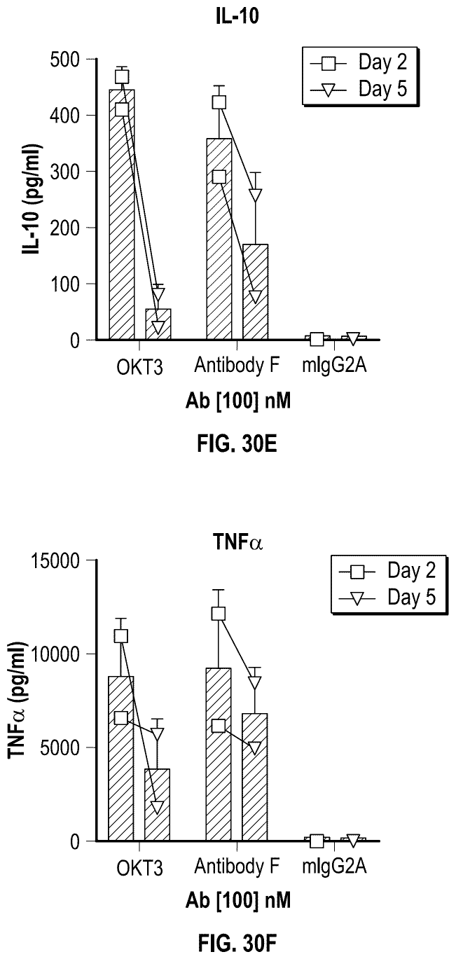

While IL-1beta (FIG. 26C), IL-6 (FIG. 26D), IL-10 (FIG. 26E), IL-4 (FIG. 26F), TNFalpha (FIG. 26G), IP-10 (FIG. 27C), IL-12-23p40 (FIG. 28D), IL-17A (FIG. 28G), and IL-1a (FIG. 28H), were induced by anti-CD3e antibody (OKT3), no or little induction of these cytokines or chemokines was observed with PBMC's activated with anti-TCRVb antibodies.

PBMCs activated with anti-TCR Vβ antibodies demonstrated induction of IL-13 (FIG. 261), IL-8 (FIG. 26J), Eotaxin (FIG. 27A), Eotaxin 3 (FIG. 27B), IL-18 (HA) (FIG. 27C), MCP-1 (FIG. 27E), MCP-4 (FIG. 27F), MDC (FIG. 27G), MIP1a (FIG. 27H), MIP1B (FIG. 28A), TARC (FIG. 28B), GM-CSF (FIG. 28C), IL-15 (FIG. 28E), IL-1β (FIG. 28F), and IL-15 (FIG. 28I), IL-7 (FIG. 28J).

### Example 11: Nanostring-Based Gene Expression Profiling of TCR Vb-Activated T Cells

This Example describes gene expression profiling of TCR Vβ-activated T cells to, e.g., uncover potential mechanisms or pathways underlying TCR Vβ activation of T cells.

In a first study, the anti-TCR Vβ13.1 antibody A-H.1 was compared with an anti-CD3 antibody OKT3. Briefly, human PBMCs were isolated from whole blood. From isolated PBMCs, human CD3+ T cells were isolated using magnetic-bead separation (negative selection) (Miltenyi biotec) and activated by immobilized (plate-coated) anti-TCR Vβ13.1 antibody (A-H.1) or anti-CD3 antibody (OKT3) at 100 nM for 6 days. Activated T-cells (from plate-coated) were then prepared for gene expression profiling (PanCancer IO 360™ Panel, nanoString), following manufacturer's protocol. Differential gene expression analysis was grouped by anti-TCR Vβ13.1 (A-H.1) vs anti-CD3 (OKT3) activated T-cells using the nSolver Analysis Software (Nanostring). Data shown in Table 16A are mean values from 3 donors. The differentially regulated genes shown in Table 16A have a p-value of 0.05 or less. In the fourth column of Table 16A showing fold changes in gene expression, a positive value indicates genes that are upregulated at the transcriptional level in TCR Vβ-activated T cells compared to OKT3-activated T cells, whereas a negative value indicates genes downregulated at the transcriptional level in TCR Vβ-activated T cells compared to OKT3-activated T cells.

In a second study, the multispecific anti-TCR Vβ13.1/anti-BCMA antibody Molecule H was compared with the anti-CD3 antibody OKT3. Purified T cells were stimulated with solid-phase anti-TCR Vβ antibody over 6 days with the anti-TCR Vβ antibody Molecule H or anti-CD3e antibody (OKT3) at 100 nM. Expanded T cells were collected by centrifugation followed by RNA extraction. Seven hundred and seventy eight (778) immunology-related genes were counted using the nCounter Technology (Nanostring) followed by gene expression analysis using nSolver analysis tools. The data described in this Example is representative of 3 donors.

Based on this analysis, a panel of genes were identified as being differentially regulated in TCR Vβ-activated T cells compared to OKT3-activated T cells (Table 16B). The differentially regulated genes shown in Table 16B have a p-value of 0.05 or less. For example, LIF, CD40LG, PDCD1, CXCR5, LTA, and CD80 are all upregulated at the transcriptional level in TCR Vβ-activated T cells compared to OKT3-activated T cells. GZMK, ENTPD1 (CD39), TCF7, CD96, HLA-DRB4, SIGIRR and SELL are downregulated at the transcriptional level in TCR Vβ-activated T cells compared to OKT3-activated T cells. TCR Vβ-activated T cells also expressed high levels of cytolytic effectors (e.g., IFNg, Granzyme B and perforin).

TABLE 16A
Summary of genes whose expression are preferentially regulated
in TCR Vβ-activated T cells compared to OKT3-activated T cells.

| | | | TCR Vβ13.1 | |
|---|---|---|---|---|
| | | | vs OKT3 | |
| Probe Name | Accession # | NS Probe ID | Fold Change | P value |
| CCR2 | NM_001123041.2 | NM_001123041.2:743 | −3.06 | 0.00019145 |
| LIF | NM_002309.3 | NM_002309.3:1240 | 21.6 | 0.0003319 |
| TCF7 | NM_003202.2 | NM_003202.2:2420 | −8.38 | 0.00037035 |
| PLA2G6 | NM_001004426.1 | NM_001004426.1:1954 | −2.19 | 0.00043564 |
| CD84 | NM_001184879.1 | NM_001184879.1:28 | −3.81 | 0.00062413 |
| ITGB2 | NM_000211.2 | NM_000211.2:520 | −2.11 | 0.0012003 |
| GZMK | NM. 002104.2 | NM_002104.2:700 | −11.09 | 0.00135083 |
| HLA-DRB4 | NM_021983.4 | NM_021983.4:194 | −5.75 | 0.00137591 |
| CCR7 | NM_001838.2 | NM_001838.2:1610 | −2.43 | 0.00165716 |
| PDCD1 | NM_005018.1 | NM_005018.1:175 | 7.24 | 0.00195468 |
| CD96 | NM_005816.4 | NM_005816.4:1355 | −6.44 | 0.00221401 |
| SELL | NR_029467.1 | NR_029467.1:1585 | −5 | 0.00227156 |
| NFATC4 | NM_001136022.2 | NM_001136022.2:2296 | −2.75 | 0.0025171 |
| CD8B | NM_004931.3 | NM_004931.3:440 | −3.56 | 0.00302475 |
| NLRC5 | NM_032206.4 | NM_032206.4:860 | −2.27 | 0.00309164 |
| CD1C | NM_001765.2 | NM_001765.2:750 | 8.62 | 0.0035729 |
| HLA-B | NM_005514.6 | NM_005514.6:937 | −1.81 | 0.00363669 |
| NUP107 | NM_020401.2 | NM_020401.2:1002 | 1.64 | 0.00366886 |
| CD3D | NM_000732.4 | NM_000732.4:110 | −2.05 | 0.00401569 |
| HDAC3 | NM_003883.2 | NM_003883.2:1455 | −1.41 | 0.0042794 |
| PRKCE | NM_005400.2 | NM_005400.2:1695 | −1.86 | 0.00429076 |
| HLA-DQB1 | NM_002123.3 | NM_002123.3:384 | −5.71 | 0.00430297 |
| AKT3 | NM_181690.1 | NM_181690.1:755 | −2.98 | 0.00430433 |
| VCAM1 | NM_001078.3 | NM_001078.3:2535 | −23.93 | 0.00464703 |
| CD53 | NM_001040033.1 | NM_001040033.1:835 | −1.7 | 0.00507702 |
| LRP1 | NM_002332.2 | NM_002332.2:4240 | −2.22 | 0.00508974 |
| CD28 | NM_001243078.1 | NM_001243078.1:2065 | −1.73 | 0.00545641 |
| OSM | NM_020530.4 | NM_020530.4:580 | 8.97 | 0.00558554 |
| CLEC4A | NM_194448.2 | NM_194448.2:388 | −1.7 | 0.0056661 |
| MFGE8 | NM_001114614.1 | NM_001114614.1:328 | −2.75 | 0.00633707 |
| IFNAR2 | NM_000874.3 | NM_000874.3:631 | −3.69 | 0.00659279 |
| LTA | NM_000595.2 | NM_000595.2:885 | 6.53 | 0.00727884 |
| ITGAE | NM_002208.4 | NM_002208.4:3405 | −3.42 | 0.00779862 |
| CXCR5 | NM_001716.3 | NM_001716.3:2618 | 4.38 | 0.00781195 |
| CD6 | NM_006725.3 | NM_006725.3:1280 | −1.38 | 0.00848703 |
| ICOS | NM_012092.2 | NM_012092.2:640 | 1.74 | 0.00914866 |
| NOS2A | NM_153292.1 | NM_153292.1:546 | −2.29 | 0.0095337 |
| CD1A | NM_001763.2 | NM_001763.2:1815 | 5.12 | 0.00956367 |
| CD27 | NM_001242.4 | NM_001242.4:330 | −3.41 | 0.00984676 |
| KLRD1 | NM_002262.3 | NM_002262.3:542 | −6.43 | 0.00998325 |
| TARP | NM_001003799.1 | NM_001003799.1:560 | −3.71 | 0.00998698 |
| HLA-DPB1 | NM_002121.4 | NM_002121.4:931 | −8.85 | 0.01064161 |
| PTPRC | NM_080921.3 | NM_080921.3:258 | −2.86 | 0.01124117 |
| CD44 | NM_001001392.1 | NM_001001392.1:429 | −2.07 | 0.01138242 |
| SLAMF6 | NM_001184714.1 | NM_001184714.1:1032 | −1.81 | 0.00175123 |
| HLA-DMB | NM_002118.3 | NM_002118.3:20 | −6.39 | 0.01184625 |
| CD276 | NM_001024736.1 | NM_001024736.1:2120 | 6.22 | 0.01207813 |
| MAGEA1 | NM_004988.4 | NM_004988.4:476 | −2.93 | 0.01210408 |
| HLA-DMA | NM_006120.3 | NM_006120.3:380 | −5.75 | 0.1210789 |
| EP300 | NM_001429.2 | NM_001429.2:715 | −1.24 | 0.01228626 |
| ADA | NM_000022.2 | NM_000022.2:1300 | −2.97 | 0.01228787 |
| CAM1 | NM_000201.2 | NM_000201.2:2253 | 2.52 | 0.01290081 |
| SIGIRR | NM_021805.2 | NM_021805.2:469 | −4.46 | 0.01309473 |
| TNF | NM_000594.2 | NM_000594.2:1010 | 4.6 | 0.01318389 |
| IL1RAP | NM_002182.2 | NM_002182.2:460 | 2.77 | 0.01329693 |
| CSF1 | NM_000757.4 | NM_000757.4:823 | 2.55 | 0.01373637 |
| CD40LG | NM_000074.2 | NM_000074.2:1225 | 11.92 | 0.01376174 |
| CYFIP2 | NM_001037332.2 | NM_001037332.2:4043 | −1.38 | 0.01389707 |
| MUC1 | NM_001018017.1 | NM_001018017.1:725 | 3.12 | 0.01399543 |
| HLA-DRB3 | NM_022555.3 | NM_022555.3:698 | −7.11 | 0.01404049 |
| CD2 | NM_001767.3 | NM_001767.3:687 | −1.53 | 0.01432842 |
| IL2RG | NM_000206.1 | NM_000206.1:595 | −1.82 | 0.01477006 |
| HLA-A | NM_002116.5 | NM_002116.5:1000 | −1.96 | 0.01454336 |
| TXK | NM_003328.1 | NM_003328.1:800 | −2.7 | 0.01590341 |
| ITGA4 | NM_000885.4 | NM_000885.4:975 | −3.59 | 0.01601785 |
| DHX16 | NM_001164239.1 | NM_001164239.1:2490 | 1.41 | 0.0167432 |
| CD3E | NM_000733.2 | NM_000733.2:75 | −1.52 | 0.01736902 |
| MR1 | NM_001531.2 | NM_001531.2:7695 | −2.26 | 0.01744764 |
| SMAD3 | NM_005902.3 | NM_005902.3:4220 | −2.82 | 0.01751245 |
| CCRL2 | NM_003965.4 | NM_003965.4:1110 | −1.87 | 0.01834479 |
| HRAS | NM_005343.2 | NM_005343.2:396 | 1.97 | 0.0187379 |
| IL18R1 | NM_003855.2 | NM_003855.2:2025 | 2.36 | 0.01896204 |
| CMA1 | NM_001836.2 | NM_001836.2:561 | −1.96 | 0.01964938 |
| PSMB7 | NM_002799.2 | NM_002799.2:420 | 1.53 | 0.01980367 |
| BCL 10 | NM_003921.2 | NM_003921.2:1250 | −1.38 | 0.01981376 |
| HLA-DRA | NM_019111.3 | NM_019111.3:335 | −7.46 | 0.02026993 |
| CD80 | NM_005191.3 | NM_005191.3:1288 | 4.18 | 0.02055337 |
| PIK3CD | NM_005026.3 | NM_005026.3:2978 | −1.23 | 0.02056576 |
| ETS1 | NM_005238.3 | NM_005238.3:4625 | −1.51 | 0.02083359 |
| CHUK | NM_001278.3 | NM_001278.3:860 | 1.67 | 0.0217326 |
| CCL5 | NM_002985.2 | NM_002985.2:280 | −2.47 | 0.02195802 |
| ITGAL | NM_002209.2 | NM_002209.2:3905 | −3 | 0.02244779 |
| TNFRSF18 | NM_004195.2 | NM_004195.2:445 | −3.76 | 0.02330885 |
| EIF2B4 | NM_172195.3 | NM_172195.3:1390 | 1.28 | 0.02349098 |
| CD79A | NM_001783.3 | NM_001783.3:695 | −4.47 | 0.02361746 |
| ABCF1 | NM_001090.2 | NM_001090.2:850 | 1.31 | 0.02452054 |
| CD37 | NM_001774.2 | NM_001774.2:535 | −2.06 | 0.02476513 |
| STAT5B | NM_012448.3 | NM_012448.3:200 | −1.56 | 0.02495121 |
| CSF2 | NM_000758.2 | NM_000758.2:475 | 11.38 | 0.0256982 |
| STAT3 | NM_139276.2 | NM_139276.2:4535 | −1.47 | 0.02629936 |
| GZMA | NM_006144.2 | NM_006144.2:155 | −2.46 | 0.02646368 |
| C1R | NM_001733.4 | NM_001733.4:760 | −3.1 | 0.02653879 |
| MIF | NM_002415.1 | NM_002415.1:319 | −1.38 | 0.02690018 |
| CD46 | NM_172350.1 | NM_172350.1:365 | −1.36 | 0.02725208 |
| PIK3CG | NM_002649.2 | NM_002649.2:2125 | −2.34 | 0.02762105 |
| CFB | NM_001710.5 | NM_001710.5:2029 | −2.59 | 0.02802998 |
| IL3 | NM_000588.3 | NM_000588.3:130 | 13.37 | 0.02820076 |
| TNFRSF13C | NM_052945.3 | NM_052945.3:789 | −2.2 | 0.02835259 |
| MRPS5 | NM_031902.3 | NM_031902.3:390 | 1.2 | 0.02849936 |
| TUBB | NM_178014.2 | NM_178014.2:320 | 1.06 | 0.02874459 |
| PECAM1 | NM_000442.3 | NM_000442.3:1365 | −4.35 | 0.02901845 |
| PVR | NM_006505.3 | NM_006505.3:604 | 2.28 | 0.0299334 |
| AMICA1 | NM_153206.2 | NM_153206.2:620 | −2.38 | 0.03034954 |
| CD74 | NM_001025159.1 | NM_001025159.1:964 | −3.28 | 0.0305419 |
| ENTPD1 | NM_001098175.1 | NM_001098175.1:8830 | −8.02 | 0.03085618 |
| CD97 | NM_078481.2 | NM_078481.2:1370 | −1.56 | 0.03086014 |
| KLRK1 | NM_007360.3 | NM_007360.3:522 | −4.16 | 0.03108504 |
| HLA-DQA1 | NM_002122.3 | NM_002122.3:261 | −5.51 | 0.03126291 |
| CD247 | NM_198053.1 | NM_198053.1:1490 | −1.88 | 0.03182703 |
| IFNG | NM_000619.2 | NM_000619.2:970 | 5.98 | 0.03202586 |
| SAA1 | NM_199161.1 | NM_199161.1:135 | −2.35 | 0.03341258 |
| TBX21 | NM_013351.1 | NM_013351.1:890 | 1.92 | 0.03359165 |
| RORA | NM_134261.2 | NM_134261.2:1715 | −2.57 | 0.03591525 |
| MASP2 | NM_139208.1 | NM_139208.1:330 | −1.65 | 0.03611762 |
| CLU | NM_001831.2 | NM_001831.2:2340 | −1.55 | 0.0369776 |
| KLRB1 | NM_002258.2 | NM_002258.2:85 | −7.43 | 0.03705134 |
| RELA | NM_021975.2 | NM_021975.2:360 | −1.26 | 0.03765981 |
| SLAMF1 | NM_003037.2 | NM_003037.2:580 | 1.82 | 0.03768168 |
| CD8A | NM_001768.5 | NM_001768.5:1320 | −4.49 | 0.0380276 |
| ILIIRA | NM_147162.1 | NM_147162.1:400 | −3.54 | 0.03855863 |
| CD3G | NM_000073.2 | NM_000073.2:404 | −1.44 | 0.03877635 |
| JAK1 | NM_002227.1 | NM_002227.1:285 | −1.84 | 0.4001383 |
| SPN | NM_003123.3 | NM_003123.3:2345 | −1.72 | 0.04035383 |
| CXCR4 | NM_003467.2 | NM_003467.2:1335 | −3.03 | 0.04122601 |
| FAS | NM_000043.3 | NM_000043.3:90 | −2.37 | 0.04150638 |
| IL2 | NM_000586.2 | NM_000586.2:300 | 10.9 | 0.04175377 |
| ITGA1 | NM_181501.1 | NM_181501.1:1875 | −2.75 | 0.04213304 |
| IGFIR | NM_000875.2 | NM_000875.2:455 | −1.94 | 0.0424234 |
| CLEC6A | NM_001007033.1 | NM_001007033.1:342 | −2.83 | 0.04299769 |
| RPS6 | NM_001010.2 | NM_001010.2:171 | −1.36 | 0.04334091 |
| MAPK11 | NM_002751.5 | NM_002751.5:1310 | −1.98 | 0.04344288 |
| REL | NM_002908.2 | NM_002908.2:225 | −2.37 | 0.04382344 |
| EOMES | NM_005442.2 | NM_005442.2:1670 | −6.49 | 0.04442535 |
| KLRG1 | NM_005810.3 | NM_005810.3:65 | −3.52 | 0.04487411 |
| IL2RA | NM_000417.1 | NM_000417.1:1000 | 3.4 | 0.0457568 |
| IFNA17 | NM_021268.2 | NM_021268.2:291 | −3.13 | 0.04595868 |
| SH2D1B | NM_053282.4 | NM_053282.4:545 | −1.44 | 0.04640447 |
| CCL2 | NM_002982.3 | NM_002982.3:123 | 4.01 | 0.04660539 |
| TXNIP | NM_006472.1 | NM_006472.1:255 | −4.07 | 0.04695375 |
| CXCL13 | NM_006419.2 | NM_006419.2:210 | −65.05 | 0.04708191 |
| CASP8 | NM_001228.4 | NM_001228.4:301 | −1.42 | 0.04720592 |
| MTMR14 | NM_022485.3 | NM_022485.3:720 | −1.25 | 0.04798024 |
| MAP3K5 | NM_005923.3 | NM_005923.3:1760 | −1.62 | 0.04838454 |
| ADORA2A | NM_000675.3 | NM_000675.3:1095 | 1.3 | 0.04872028 |
| CCRS | NM_000579.1 | NM_000579.1:2730 | −4.01 | 0.04885927 |

TABLE 16B
Summary of genes whose expression are preferentially regulated in
TCR Vβ-activated T cells compared to OKT3-activated T cells.

| | | Log₂ | |
|---|---|---|---|
| | | Fold | |
| Gene | Description | Change | P-Value |
| LIF | LIF Interleukin 6 Family Cytokine | 4.65 | 0.0119 |
| GZMK | Granzyme K | −3.65 | 0.0468 |
| CD40LG | CD40 Ligand | 3.56 | 0.0082 |
| ENTPD1 | Ectonucleoside Triphosphate | −3.53 | 0.0541 |
| (CD39) | Diphosphohydrolase 1 | | |
| PDCD1 | Programmed Cell Death 1 | 3.19 | 0.0257 |
| TCF7 | Transcription Factor 7 | −3.1 | 0.00634 |
| CXCR5 | Chemokine receptor for CXCL 13 | 3.05 | 0.0337 |
| CD96 | Transmembrane | −2.75 | 0.007 |
| | glycoprotein Ig superfamily | | |
| | receptor, interacts with | | |
| | nectin and nectin-like | | |
| | proteins, including | | |
| | CD155/polio virus receptor | | |
| | (PVR) | | |
| LTA | Lymphotoxin Alpha | 2.67 | 0.0082 |
| HLA-DRB4 | Major Histocompatibility | −2.66 | 0.0377 |
| | Complex, Class II, DR | | |
| | Beta 4 | | |
| CD80 | T cell costimulatory molecule | 2.58 | 0.0425 |
| SIGIRR | Single Ig And TIR Domain Containing | −2.37 | 0.0227 |
| SELL | Selection L | −2.3 | 0.00634 |

### Example 12: Binding Affinity of Affinity Matured Humanized Antibody A-H Antibodies

This Example describes the evaluation of binding affinity of affinity matured humanized Antibody A-H antibodies to recombinant protein TCRVB 6-5.

Antibody A-H humanized antibodies were affinity matured. The resulting affinity matured antibodies were tested for their binding affinity to TCRVB 6-5 as described below.

TCRVB 6-5 at 5 ug/mL was immobilized on a Biotin CAP Series S Sensor Chip to 60 RU. BJM0277 was diluted to 200 nM and then serially diluted two fold. Association was 120 seconds, and dissociation was 300 seconds. This assay was run in 1×HBS-EP+ Buffer pH 7.4 and 25C. The data was fit using a 1:1 binding model.

TCRVB 6-5 at 5 ug/mL was immobilized on a Biotin CAP Series S Sensor Chip to 60 RU. A-H.45 was diluted to 50 nM and then serially diluted two fold. Association was 120 seconds, and dissociation was 300 seconds. This assay was run in 1×HBS-EP+ Buffer pH 7.4 and 25C. The data was fit using a 1:1 binding model. A-H.45 is an improved yeast clone (TCRvB/CD 19 bispecific) and contains a mutation (G to V) at the last residue in framework 3, just before HCDR3. The affinity is 35-fold greater than the BJM0277 (Table 17).

TCRVB 6-5 at 5 ug/mL was immobilized on a Biotin CAP Series S Sensor Chip to 60 RU. A-H.52 was diluted to 50 nM and then serially diluted two fold Association was 120 seconds, dissociation was 300 seconds. This assay was run in 1×HBS-EP+ Buffer pH 7.4 and 25C. The data was fit using a 1:1 binding model. A-H.52 is a phage clones and is a monovalent scFv. A-H.52 has two mutations on CDRH1. The affinity of A-H.52 is 20-fold greater than BJM0277 (Table 17).

TCRVB 6-5 at 5 ug/mL was immobilized on a Biotin CAP Series S Sensor Chip to 60 RU. A-H.53 was diluted to 50 nM and then serially diluted two fold. Association was 120 seconds, dissociation was 300 seconds. This assay was run in 1×HBS-EP+ Buffer pH 7.4 and 25C. The data was fit using a 1:1 binding model. A-H.53 (phage clone) affinity is in the pM range (Table 17). The affinity of A-H.53 is 200-fold greater than BJM0277 (Table 17).

TCRVB 6-5 at 5 ug/mL was immobilized on a Biotin CAP Series S Sensor Chip to 60 RU. A-H.54 was diluted to 50 nM and then serially diluted two fold. Association was 120 seconds, dissociation was 300 seconds. This assay was run in 1×HBS-EP+ Buffer pH 7.4 and 25C. The data was fit using a 1:1 binding model. A-H.54 (phage clone) affinity is 17-fold greater than BJM0277 (Table 17).

TABLE 17
Summary of affinity maturation of anti-TCRVb antibodies

| Construct | Target: TCRVβ 6-5 |
|---|---|
| BJM0277 | 35 nM |
| A-H.45 | 1.08 Nm |
| A-H.52 | 1.76 nM |
| A-H.53 | 165 pM |
| A-H.54 | 2.22 nM |

### Example 13: Expression and Purification of Antibody Constructs

### Construction of the Plasmids

The DNA encoding the protein sequences was optimized for expression in Cricetulus griseus, synthesized, and cloned into the peDNA3.4-TOPO (Life Technologies A14697) using Gateway cloning. All constructs contained an Ig Kappa leader sequence METDTLLLWVLLLWVPGSTG (SEQ ID NO: 3288).

### Expression and Purification

The plasmids were co-transfected into either Expi293 cells (Life Technologies A 14527) or ExpiCHO cells (Life Technologies A29127). Transfections were performed using 1 mg of total DNA for a multispecific construct with a 1:1 heavy chain ratio and 3:2 light chain to heavy chain ratio if applicable. Transfection in Expi293 cells was done using linear 25.000 Da polyethylenimine (PEI, Polysciences Inc 23966) in a 3:1 ratio with the total DNA. The DNA and PEI were each added to 50 mL of OptiMem (Life Technologies 31985088) medium and sterile filtered. The DNA and PEI were combined for 10 minutes and added to the Expi293 cells with a cell density of 1.8-2.8×10⁶ cells/mL and a viability of at least 95%. The ExpiCHO transfection was performed according to the manufacturer's instructions. Expi293 cells were grown in a humidified incubator at 37° C., with 8% CO₂ for 5-7 days after transfection and ExpiCHO cells were grown for 14 days at 32° C., with 5% CO₂. The cells were pelleted by centrifugation at 4500 kg and the supernatant was filtered through a 0.2 μm membrane. Protein A resin (GE 17-1279-03) was added to the filtered supernatant and incubated for 1-3 hours at room temperature. The resin was packed into a column, washed with 3×10 column volumes of Dulbecco's phosphate-buffered saline (DPBS, Life Technologies 14190-144). The bound protein was eluted from the column with 20 mM citrate. 100 mM NaCl, pH 2.9. When necessary, the proteins were further purified using ligand affinity and/or size exclusion chromatography on a Superdex 200 column with a running buffer of DPBS.

### Example 14: Humanization of Anti-TRBV5-5 Antibody Clone Antibody C

The germline for the mouse anti-TCRvbeta antibody clone Antibody C VH and VL were assigned using IMGT nomenclature, with CDR regions defined by a combined Kabat and Chothia classification, SEQ ID NO: 232 and SEQ ID NO: 233 are the Antibody C VH and VL sequences respectively where the VH germline is mouse IGHV2-6-7*01 and the VL germline is mouse IGKV10-94*02. The method applied to humanize Antibody A described in Example 1 was used to humanize Antibody C. The Antibody C VH was humanized into human IGHV2-26*01, IGHV2-70*04, IGHV4-4*02, IGHV2-5*09, IGHV2-5*08, IGHV4-34*09, IGHV4-59*01, IGHV4-59*07, IGHV4-61*02, IGHV4-38-2*01, IGHV4-31*01, IGHV3-49*04, IGHV3-49*02, IGHV4-4*07, IGHV3-49*05, IGHV4-34*10, IGHV4-28*04, IGHV3-72*01, IGHV3-15*07. IGHV6-1*01, IGHV3-7*01, IGHV4-34*01, IGHV3-33*02, IGHV3-48*02, IGHV3-23*03, IGHV3-21*01, IGHV3-73*01, IGHV3-30*02, IGHV3-7*01, IGHV3-43*01, and IGHV3-53*03 and the Antibody C VL was humanized into human IGKVID-43*01, IGKV1-27*01, IGKV1-17*02, IGKV1-17*01, IGKV1-5*01, IGKV4-1*01, IGKV3-7*02, IGKV3-7*01, IGKV2-29*02, IGKV6D-41*01, IGKV2-28*01, IGKV2-40*01, IGKV3-15*01, IGKV2-24*01, IGKV6-21*01, IGKV2D-26*01, and IGKV2D-26*03.

SEQ ID NOs: 3040-3089 are the Antibody C humanized heavy chains and SEQ ID NOs: 3000-3039 are the Antibody C humanized light chains (as described in Table 10B).

### Example 15: Humanization of TRBV10-1, TRBV10-2, and TRBV10-3 Antibody Clone Antibody D

The germline for the mouse anti-TCRvbeta antibody clone Antibody D VH and VL were assigned using IMGT nomenclature, with CDR regions defined by a combined Kabat and Chothia classification, SEQ ID NO: 3183 and SEQ ID NO: 3184 are the Antibody D VH and VL sequences respectively where the VH germline is mouse IGHV5-6*01 and the VL germline is mouse IGKV4-59*01.

The method applied to humanize Antibody A described in Example 1 was used to humanize Antibody D. The Antibody D VH was humanized into human IGHV3-30*03, IGHV3-30*02, IGHV3-7*01. IGHV3-21*01, IGHV3-23*04, IGHV3-30*15, IGHV3-48*02, IGHV3-53*04, IGHV3-23*03, IGHV3-53*03. IGHV3-53*01, IGHV3-9*01, IGHV3-30*13, IGHV3-20*01, IGHV3-43D*03, IGHV3-43*02, IGHV3-43*01, IGHV3-53*02, IGHV3-13*01, IGHV3-38-3*01, IGHV3-9*03, IGHV3-64D*06, IGHV3-33*02, IGHV3-11*03, IGHV3-64*02, IGHV3-64*01, IGHV3-64*03, IGHV3-7*01, IGHV3-35*01, IGHV3-13*02, IGHV3-38*02, and IGHV3-38*01 and the Antibody D VL was humanized into human IGKV3-11*01, IGKV1-13*02, IGKV1-9*01, IGKV6-21*01, IGKV1D-43*01, IGKV3-11*01, IGKV3D-11*02, IGKV1-17*03, IGKV3D-20*01, IGKV3-20*01, IGKV1D-16*01, IGKV4-1*01, IGKV2-28*01, IGKV2-40*01, IGKV2-29*02, IGKV2-29*01, IGKV1D-42*01, IGKV2-24*01, and IGKV5-2*01, SEQ ID NOs: 3225-3274 are the Antibody D humanized heavy chains and SEQ ID NOs: 3185-3224 are the Antibody D humanized light chains (as described in Table 12).

### Example 16: Humanization of TRBV5-5 and TRBV5-6 Antibody Clone Antibody E

The germline for the mouse anti-TCRβ antibody clone Antibody E VH and VL were assigned using IMGT nomenclature, with CDR regions defined by a combined Kabat and Chothia classification, SEQ ID NO: 3091 and SEQ ID NO: 3092 are the Antibody E VH and VL sequences respectively where the VH germline is mouse IGHV1-82*01 and the VL germline is mouse IGKV3-5*01.

The method applied to humanize Antibody A described in Example 1 was used to humanize Antibody E. The Antibody E VH was humanized into human IGHV1-69*08, IGHV1-3*02, IGHV1-18*03. IGHV1-3*01, IGHV1-18*01, IGHV1-2*06, IGHV1-2*01, IGHV1-2*06, IGHV1-8*01, IGHV7-4-1*02. IGHV1-58*02, IGHV5-51*01, IGHV7-4-1*04, IGHV7-81*01, IGHV5-51*04, IGHV5-51*01, IGHV1-45*03, IGHV3-49*04, IGHV3-49*02, IGHV3-49*05, IGHV4-4*02, IGHV3-49*05, IGHV3-73*01, IGHV4-4*02, IGHV3-15*07, IGHV3-15*02, IGHV3-72*01, IGHV4-59*07, IGHV4-31*01, IGHV4-31*02, IGHV3-30*15, IGHV3-21*01, IGHV3-7*01, IGHV4-28*01, IGHV4-28*02, IGHV3-30*08, IGHV3-30*05, and IGHV3-30*01 and the Antibody E VL was humanized into human IGKV4-1*01, IGKV3-11*01, IGKV3-20*02, IGKV3-11*01, IGKV1-13*02, IGKV3D-11*01, IGKV3D-20*02, IGKV1-13*02, IGKV3D-20*01, IGKV1-9*01, IGKV3D-15*03, IGKV3-15*01, IGKV1-5*01, IGKV2D-29*01, IGKV3-7*02, IGKV1-9*01, IGKV2-28*01, IGKV2-40*01, IGKV2D-29*02, IGKV3-7*01, IGKV2-30*01, IGKV2-24*01, IGKV6D-41*01, IGKVID-42*01, IGKV2D-26*01, IGKV2D-26*03, and IGKV5-2*01, SEQ ID NOs: 3133-3182 are the Antibody E humanized heavy chains and SEQ ID NOs: 3093-3132 are the Antibody E humanized light chains (as described in Table 11).

### Example 17: Kinetics of T Cell Expansion Following TCRβV 6-5 Stimulation

To assess the kinetics and absolute count of anti-TCRβv 6-5 expanded T cells-either PBMCs or purified T cells were stimulated with plate-immobilized anti-TCRvb 6-5 antibody over 8 days with a T cell-activating antibody at 100 nM. T cell activating antibodies tested included: i) anti-TCRvb 6-5 v1 antibody: ii) anti-TCRvb 6-5 v2; iii) OKT3 (anti-CD3ε antibody): iv) SP34-2 (anti-CD3s antibody); and v) IgG1 N297A (isotype control). Cell pellets were collected each day and stained for CD3, CD4, CD8 and TCRvb 6-5 for flow analysis.

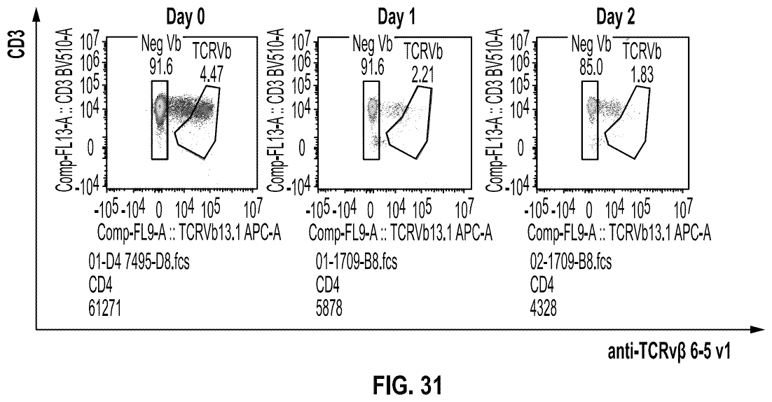

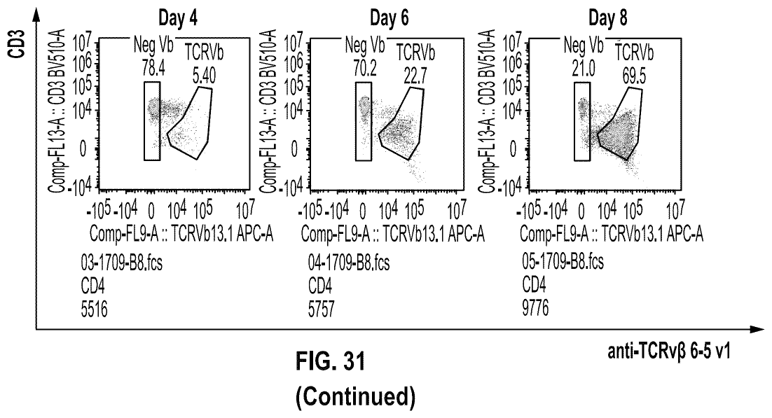

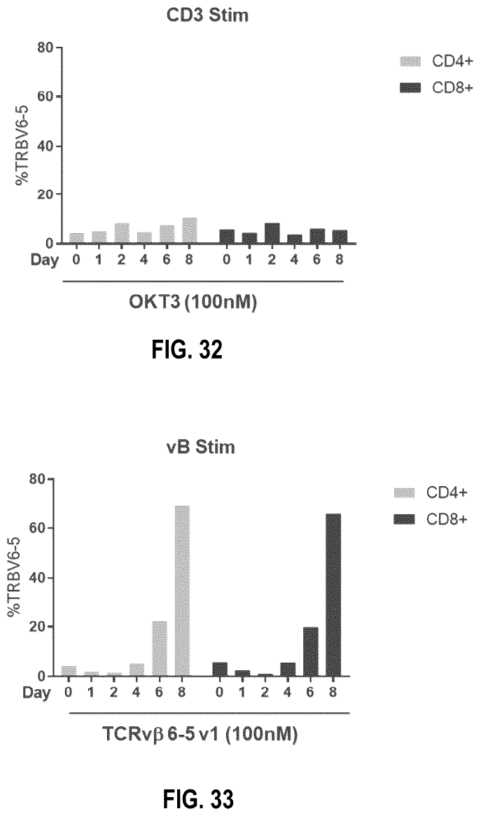

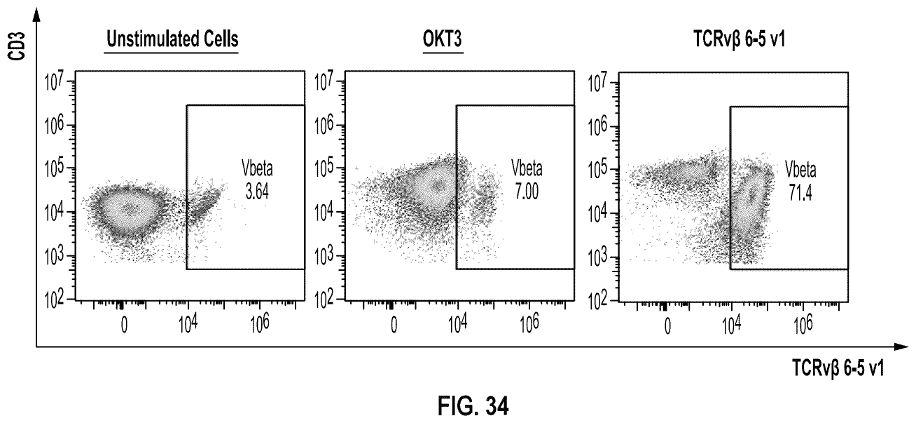

TCRvb 6-5+ T cell expansion over 8 days using anti-TCRvb 6-5 v1 is shown in FIG. 31, as assessed by flow cytometry. The data is for a single representative donor; and similar results were seen with PBMCs from two other independent donors. FIG. 33 further shows the specific expansion of TCRvb 6-5+CD4+ T cells and TCRvb 6-5+ CD8+ T cells by TCRvb 6-5 v1. In contrast, there was no specific TCRvb 6-5+ T-cell expansion by OKT3 (FIG. 32; FIG. 34).

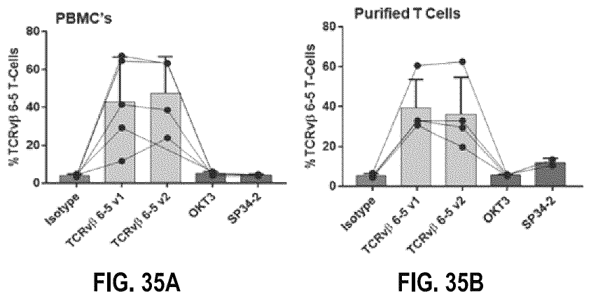

FIGS. 35A and 35B show selective expansion of TCRβV 6-5+ T cells in human PBMCs (FIG. 35A) and purified T cells (FIG. 35B).

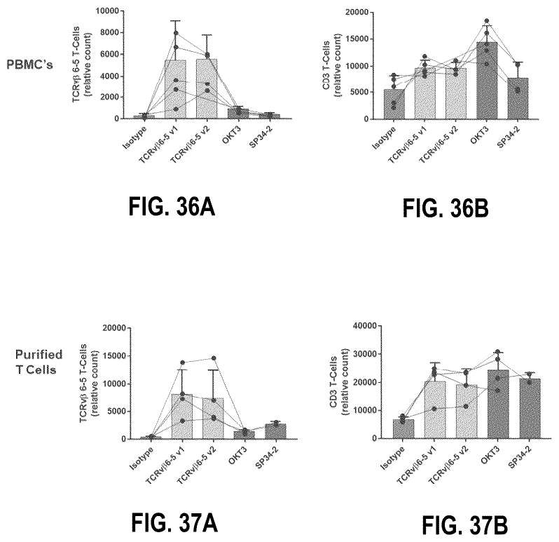

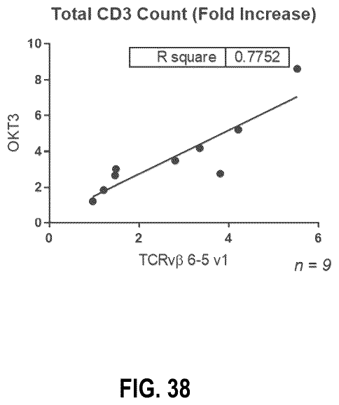

FIGS. 36A-38 shows that anti-TCRβV and anti-CD3ε antibodies expand T cells in a PBMC culture (FIGS. 36A and 36B) or a purified T cell culture (FIGS. 37A and 37B)) to comparable levels after 8 days, as measured by both relative count of TCRVB 6-5+ T cells (FIGS. 36A-37B) and relative count of total CD3+ T cells (FIGS. 36A-38).

### Example 18: Activated TCRvb 6-5+ T Cells Exert Cytolytic Function

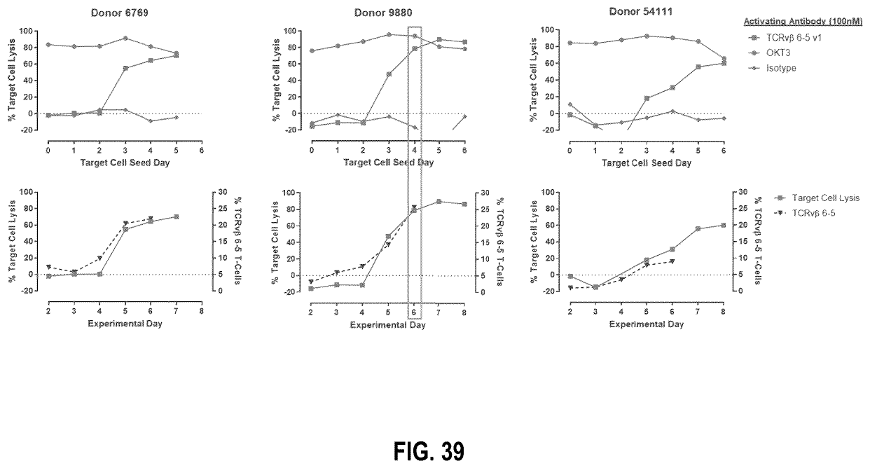

To assess the ability of T cells activated/expanded with anti-TCRVβ to mediate tumor cell lysis-purified T cells were stimulated over 6 days with an immobilized T cell-activating antibody at 100 nM. T cell activating antibodies tested included: i) TCRvb 6-5 v1 antibody: ii) OKT3 (anti-CD3ε antibody); or iii) IgG1 N297A (isotype control). Target cells (RPMI-8226 cells) were added on each day and incubated with the activated T cells at an initial effector T cell:target (E:T) cell ratio of 5:1 for 48 hours. Quantification of target cell lysis was measured using CFSE/CD138 and DRAQ7 FACS staining. Three different T cell donors were used (donor 6769, donor 9880, donor 54111). The data shows that the kinetics of target cell lysis by TCRVb 6-5 v1 activated T cells correlates with the expansion of TCRvb 6-5+ T cells (FIG. 39).

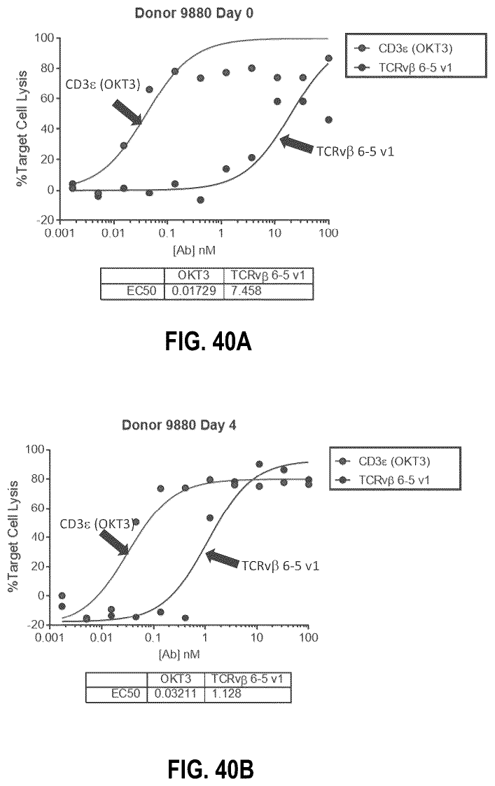

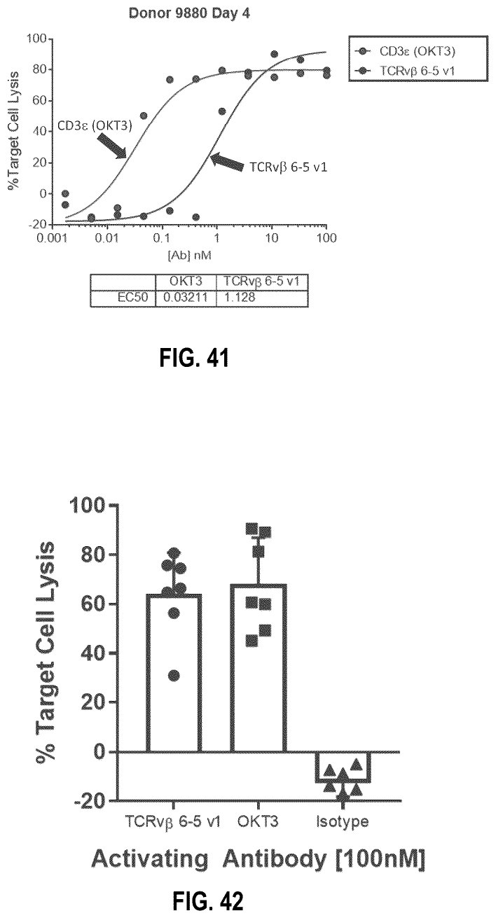

To further assess target cell lysis OKT3 or TCRvb 6-5 v1 antibodies were immobilized (plate-coated) with a ½ log serial dilution from a top dose concentration of 100 nM for purified T-cell (pan CD3 isolated) activation. The purified T-cells were stimulated with the activation plate for 0 (i.e. without antibody preactivation) to 4 (i.e. with antibody preactivation) days prior to addition of the target cells. Target cells (RPM18226) were added to the activation plate (at an initial E:T cell ratio, 5:1) for up to 6-days (i.e. for plate 0, E:T coculture for 6-days, and for plate 4, E:T coculture for 2-days) followed by target cell lysis quantification via CFSE/CD138 and DRAQ7 FACS staining. The data shows that without T-cell preactivation, approximately 3% of Vb cells were able to kill target cells at day 6 (at higher concentration) (FIG. 40A); and with T-cell preactivation, approximately 25% of Vb cells were able to kill target cells at day 6 (the killing curve is shifted to the left) (FIG. 40B). TCRvb 6-5 v1 activated T cells exhibit comparable maximal target cell lysis when compared to anti-CD3ε when T cells are preactivated for 4 days (FIG. 41). At 100 nM, TCRvb 6-5 v1 activation shows comparable killing of target cells to anti-CD3ε activation (FIG. 42) (preactivation between 4-6 days depending on the donor and the cultures cultured for 48 h in presence of target cells).

### Example 19: Assessing TCRvb Downregulation/Internalization by Anti-TCRvb 6-5 Antibody

To assess the effect anti-TCRvb 6-5 mediated T cell activation has on cell surface expression of TCRvb-purified T cells were stimulated over 8 days with the indicated T cell-activating antibody at 100 nM (plate bound). T cell activating antibodies included: i) anti-TCRvb 6-5 v1 antibody; or ii) SP34-2 (anti-CD3ε antibody). Cell pellets were collected each day and stained for CD3, CD4, CD8 and TCRβV 6-5 for flow cytometry analysis. A total of three donors were tested, each showing similar results.

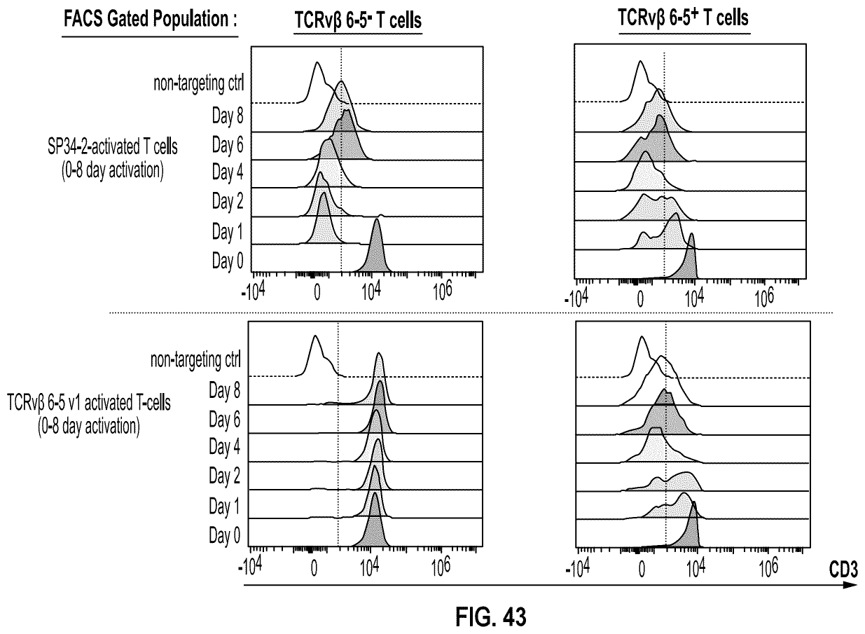

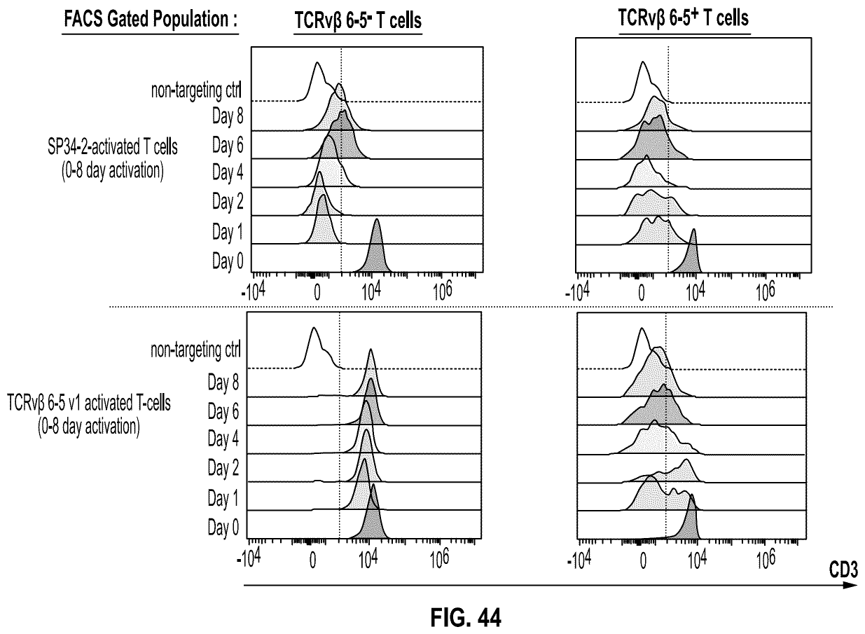

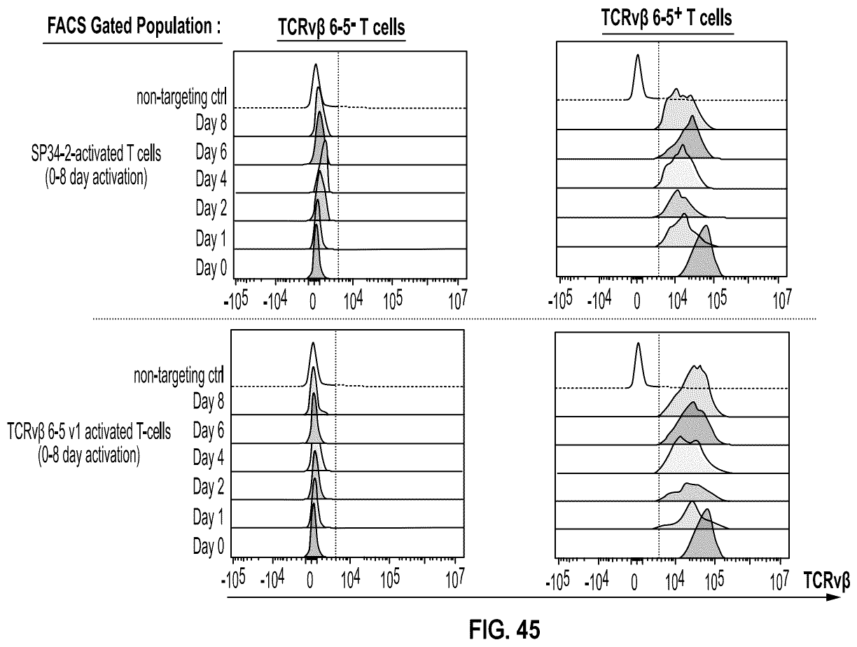

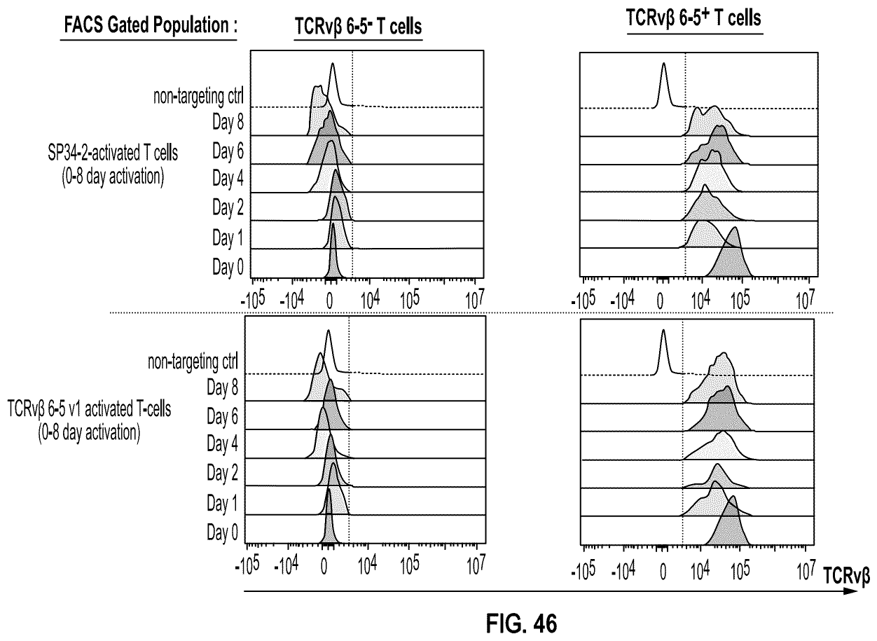

The results show that both anti-CD3ε and anti-TCRvb antibodies activated CD4+ T cells (FIG. 43) and activated CD8+ T cells (FIG. 44) display reduced CD3ε cell surface expression: whereas TCRvb 6-5 cell surface expression on CD4+ T cells (FIG. 45) and CD8+ T cells (FIG. 46) remains detectable post T cell activation. The results show that the CD3ε subunit is downregulated/internalized in T cells activated by either anti-CD3ε or anti-TCRvb antibodies: while TCRvb 6-5 remains detectable post T cell activation. Additionally, CD4 and CD8 staining did not show any signs of downmodulation of these receptors by either antibody.

### Example 20: Cynomolgus Cross Reactivity of Anti-TCRβV Antibodies

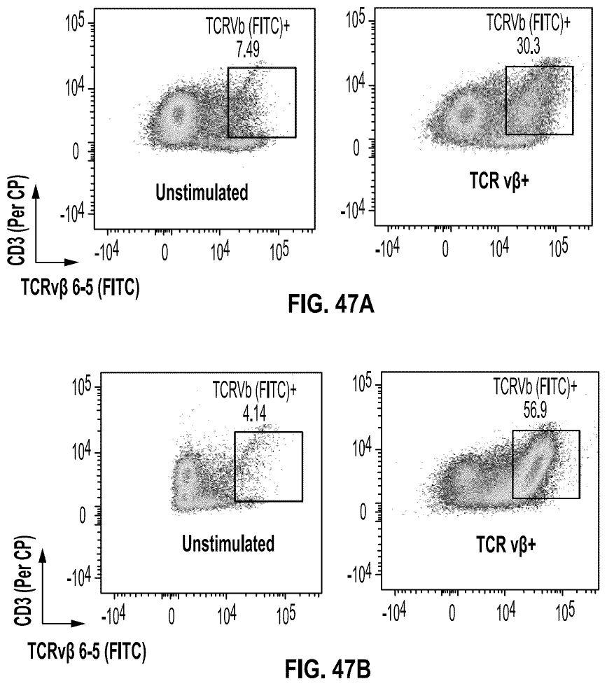

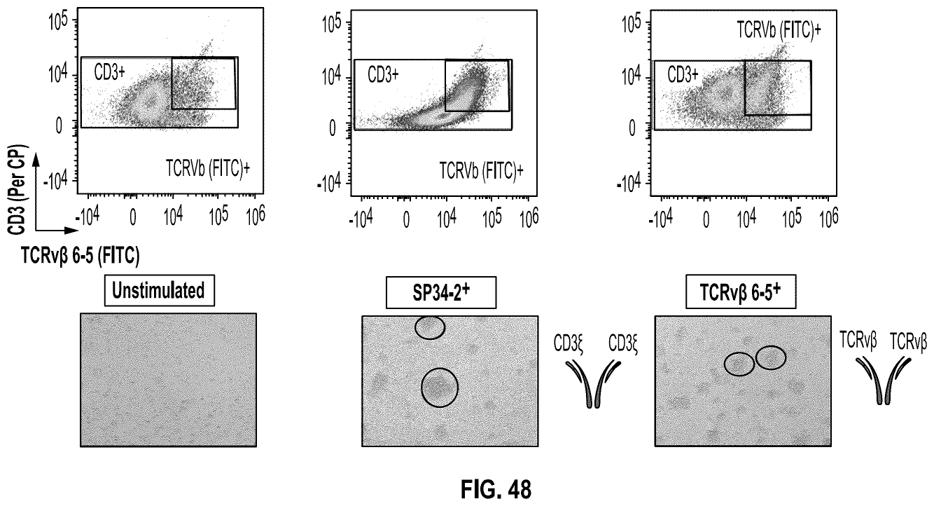

To assess the cross reactivity of anti-TCRβV antibodies for cynomolgus TCRβV clonotype—fresh and cryopreserved cynomolgus PBMCs were cultured in complete media (RPMI with 10% FBS) in tissue culture treated flat bottom 96 well plates precoated with anti-TCRβV 6-5 v1 or anti-CD3ζ antibodies at 100 nM concentration. Negative control or unstimulated wells received PBS alone. TCRβV 6-5 expression was evaluated after 6 days in culture using CytoFlex flow cytometer (Beckmann Coulter) and imaged. Two donors samples were used: Donor DW8N—fresh PBMC sample, male, age 8, weight 7.9 kgs (data presented in FIG. 47A): Donor G709-cryopreserved sample, male, age 6, weight 4.7 kgs (data presented in FIG. 47B). The data show that cynomolgus T cells were activated and expanded by the anti-TCRβV 6-5 v1 (FIG. 47A and FIG. 47B). Fresh cynomolgus PBMCs from donor DW8N that had shown TCRvb 6-5 expansion were cryopreserved and after a week in cryopreservation, the cells were thawed and stimulated using anti-CD3ξ and anti-TCRvb 6-5 v1 for seven days. Cluster formation and expansion were both reproducible as shown in FIG. 48.

### Example 21: No Activation of γδ T Cells by Anti-TCRβV Antibodies

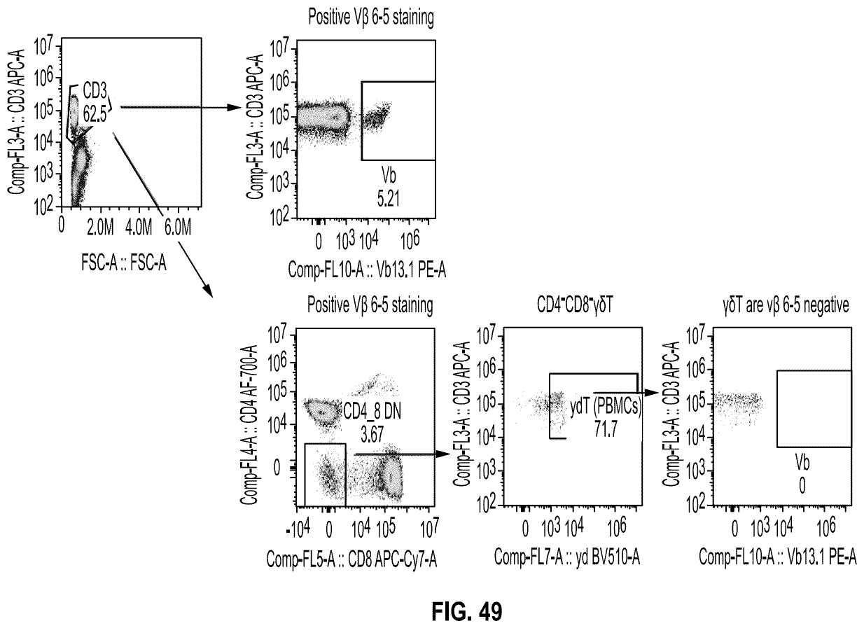

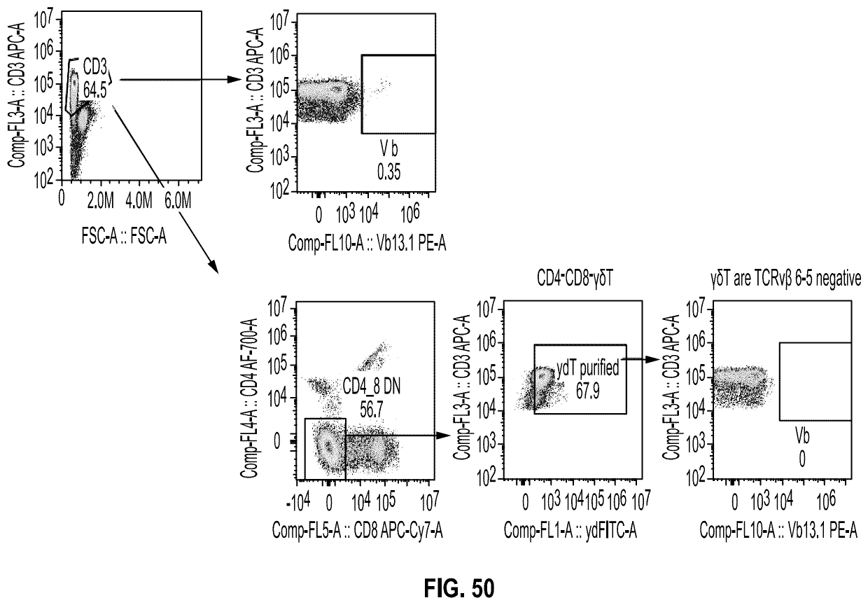

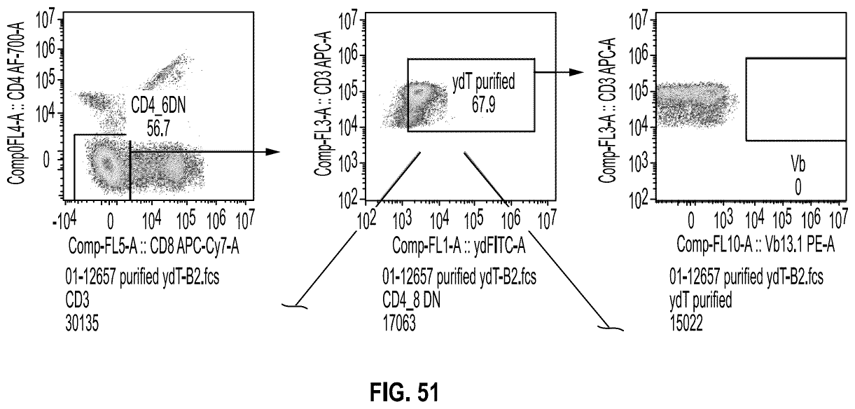

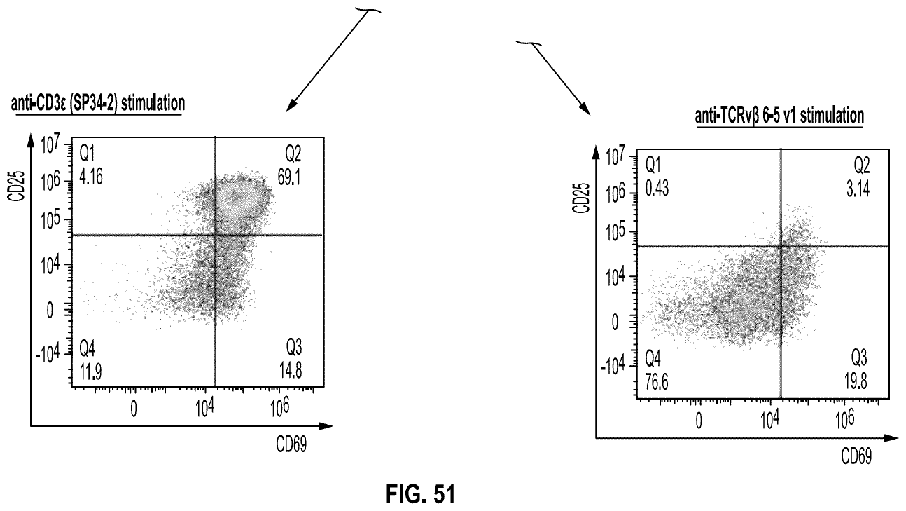

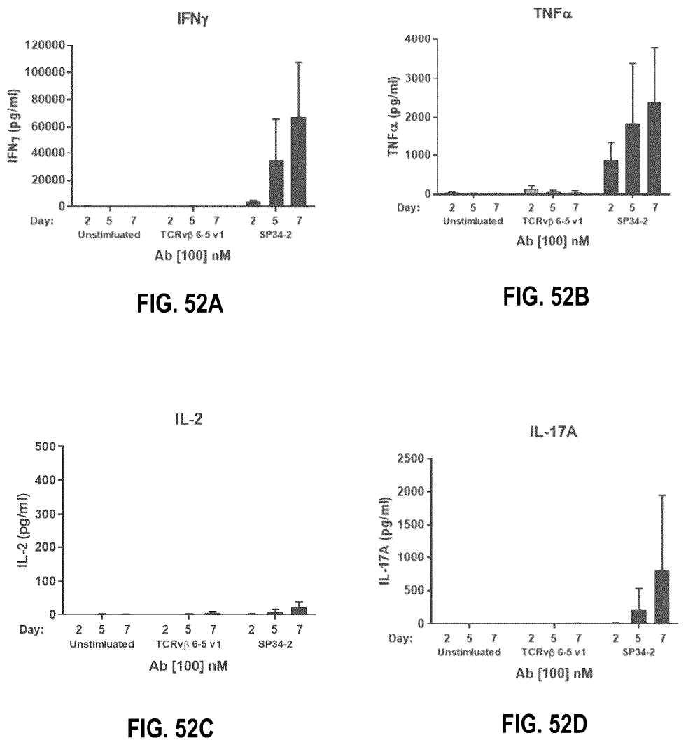

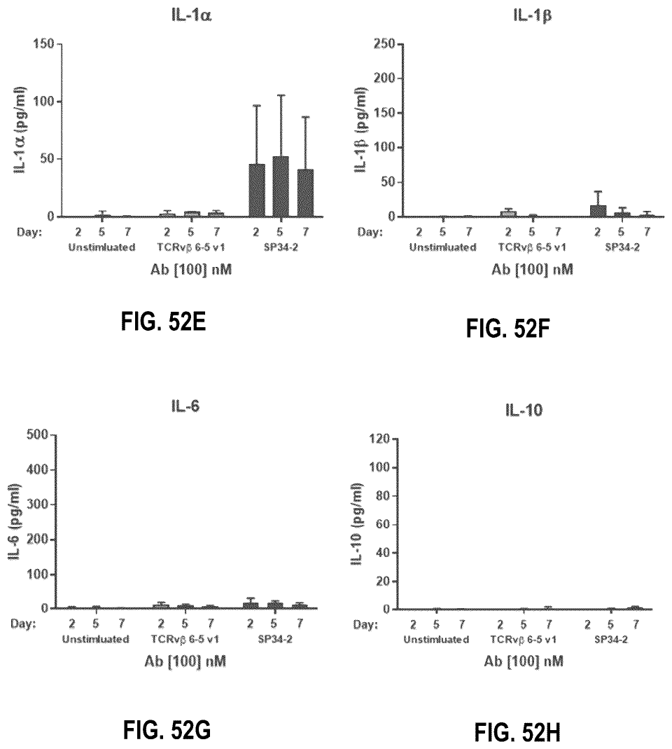

To determine if anti-TCRvb antibodies are able to activate γδ T cells-γδ T cells were purified from human PBMCs via magnetic bead separation. γδ T cells were immobilized on plate-coated anti-CD3a (SP34-2) or anti-TCRvb 6-5 (anti-TCRvb 6-5 v1) antibodies for 24 hours and analyzed for CD69 and CD25 expression by flow cytometry. Supernatants were collected post activation 2, 5, and 7 days, and analyzed for cytokines using Meso Scale Discovery (MSD) assay. FACS gating/staining on PBMCs was conducted prior to γδ T cell purification showing that γδ T cells are v 6-5 negative (Donor 12657-gating for γδ T and TCRvβ 6-5 based on FMO) (FIG. 49). FACS gating/staining on purified γδ T cell was conducted showing that purified γδ T cells are vβ 6-5 negative (Donor 12657-gating for γδ T and TCRvβ 6-5 based on FMO) (FIG. 50). As shown in FIG. 51, the anti-TCR Vβ 6-5 antibody (anti-TCRvb 6-5 v1) did not activate γδ T cells; while the anti-CD3ε antibody (SP34-2) did. The cytokine analysis showed that anti-TCRβV 6-5 v1 does not induce cytokine release by γδ T cells, cytokines analyzed include IFNγ, TNFα, IL-2, IL-17A, IL-1α, IL-1β, IL-6, and IL-10 (FIG. 52A-56H).

### Example 22: Polyclonal T Cell Expansion by Anti-TCRVβ Antibodies

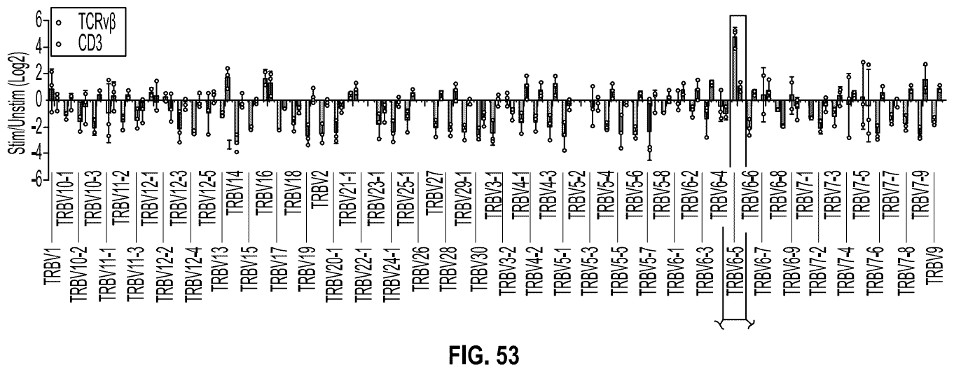

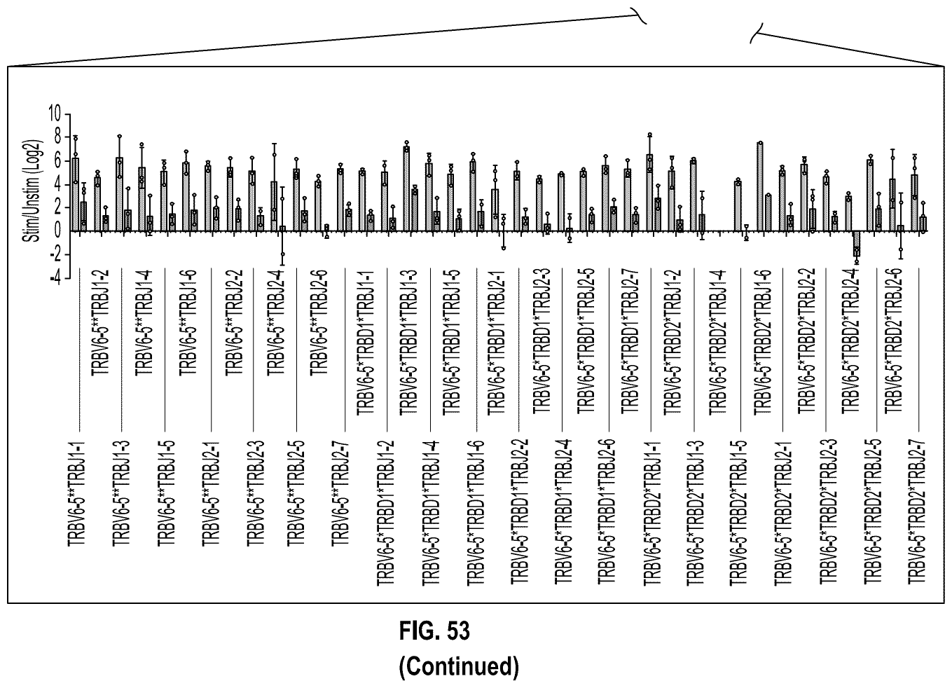

To assess the ability of anti-TCRVβ antibodies to induce polyclonal T cell expansion-human CD3+ T cells were isolated using magnetic-bead separation (negative selection) and activated with immobilized (plate-coated) anti-TCRβV 6-5 v1 at 100 DM for 6 days. The expanded T cell population was washed and lysed using Takara single cell lysis buffer for SMART(er) TCR cDNA synthesis and sequencing. TCR sequencing was carried out and absolute counts and relative representation of the different TCR alpha V and J segments and TCR beta V, D, and J segments were determined, as well as the different variants of each of them that arise from Artemis/TdT activity during the V (D) J recombination, and that correspond to unique clones of T cells. FIG. 53 shows the relative representations of all TCR alpha V segments (TRAV group of genes) and their variants (top), all TCR beta V segment 6-5 variants (TRBV6-5 gene) (bottom left), and all TCR beta V segments and variants excluding 6-5 (bottom right). The data show that the anti-TCRVβ antibody stimulation does not induce proliferation of specific T cell clones within the TRBV6-5 positive population, as the relative difference in clonal representation in that population is comparable to the TRBV6-5 negative population as well as total TRAV usage.

### Example 23: T Cells Expanded by Anti-TCRβV Represent a Novel Subset of Recently Activated Effector T Cells

To assess the phenotype of anti-TCRβV expanded T cells-purified T cells were stimulated with solid-phase anti-TCRβV antibody over 8 days with the indicated T cell-activating antibody at 100 nM: i) anti-TCRvb 6-5 v1 antibody; ii) anti-TCRvb 6-5 v2; iii) OKT3 (anti-CD3ε antibody); or iv) IgG1 N297A (isotype control). T-cell subsets were identified by FACS staining for specific surface markers for: Naive T cell (CD4/CD8+, CD45RA+, CCR7+); T stem cell memory (TSCM; CD4/CD8+, CD95+, CD45RA+, CCR7+): T central memory (TCM; CD4/CD8+, CD95+, CD45RA−, CCR7+); T effector memory (TEM; CD4/CD8+, CD95+, CD45RA−, CCR7−); T effector memory re-expressing CD45RA (TEMRA; CD4/CD8+, CD95+, CD45RA+, CCR7−); and CD27, CD28, 4-1BB, OX40, and ICOS. Data is representative of more than 5 independent experiments.

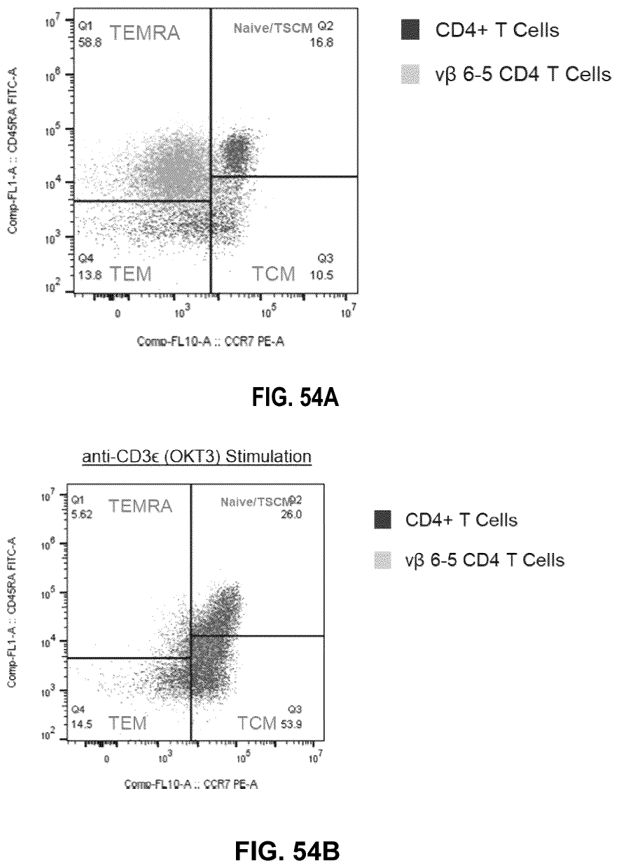

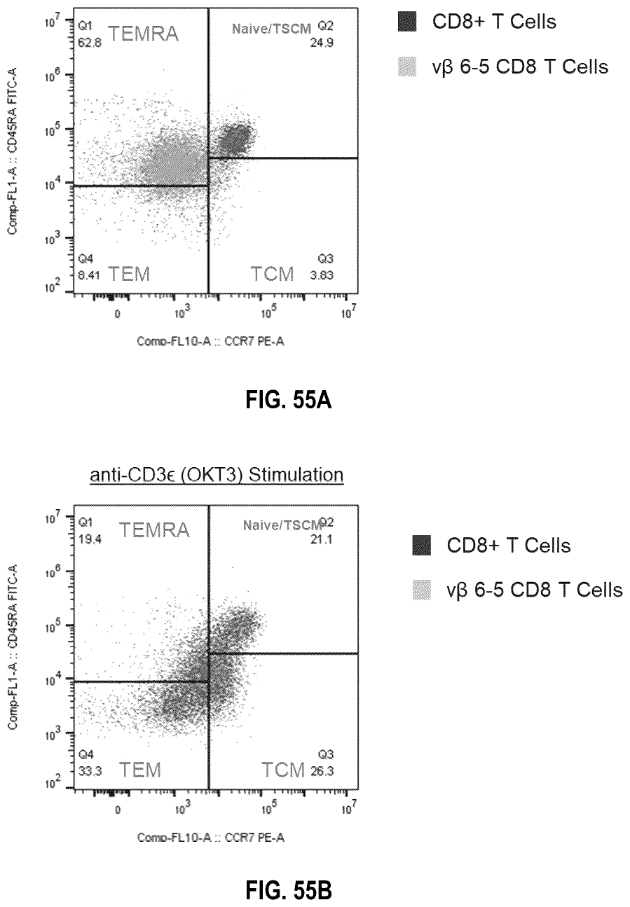

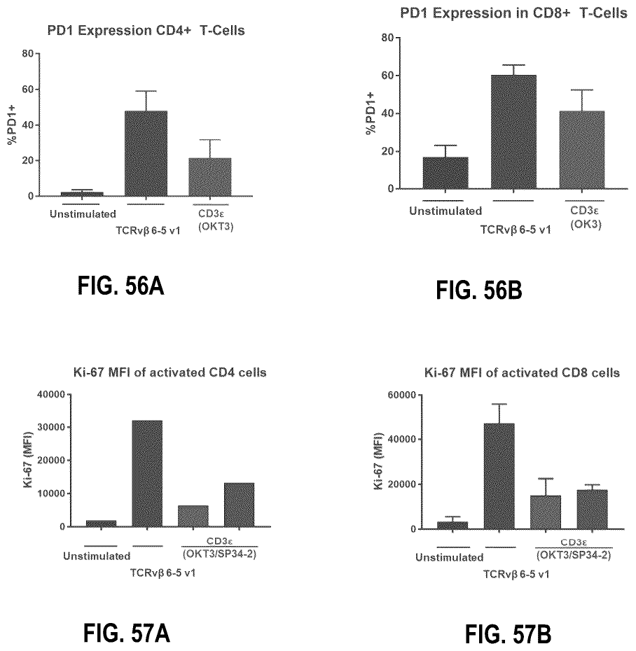

The data shows that CD4+ T cells expanded by anti-TCR Vβ antibody (FIG. 54A), but not OKT3 (FIG. 54B), share phenotypic markers with the TEMRA subset. Likewise, the data shows that CD4+ T cells expanded by anti-TCR Vβ antibody (FIG. 55A), but not OKT3 (FIG. 55B), share phenotypic markers with the T_{EMRA} subset. Further analysis of PD1 expression showed anti-TCR Vβ activated CD4+ T cells (FIG. 56A) and CD8+ T cells (FIG. 56B) display increased PD1 expression relative to anti-CD3ε activated CD4+ T cells (FIG. 56A) and CD8+ T cells (FIG. 56B). These anti-TCR Vβ activated CD4+ T cells (FIG. 57A) (PD-1+ TEMRA phenotype) and anti-TCR Vβ activated CD8+ T cells (FIG. 57B) (PD-1+ TEMRA phenotype) show Ki-67 enriched phenotype relative to anti-CD3ε activated CD4+ T cells (FIG. 57A) and CD8+ T cells (FIG. 57B).

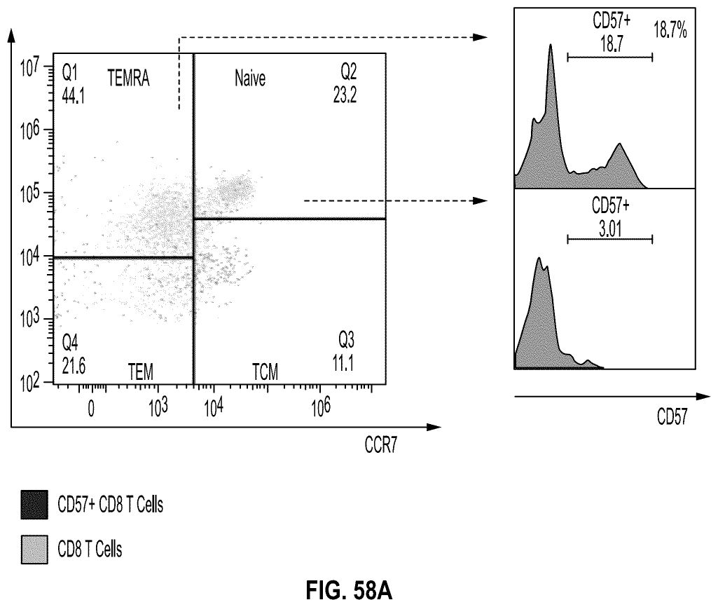

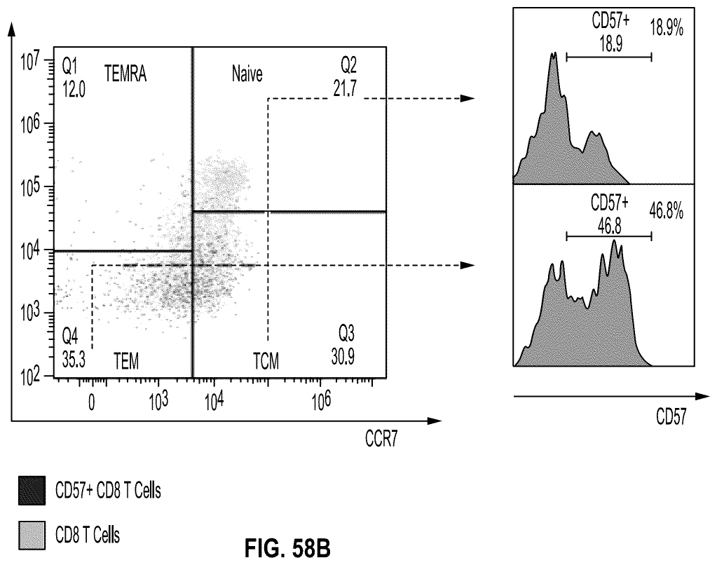

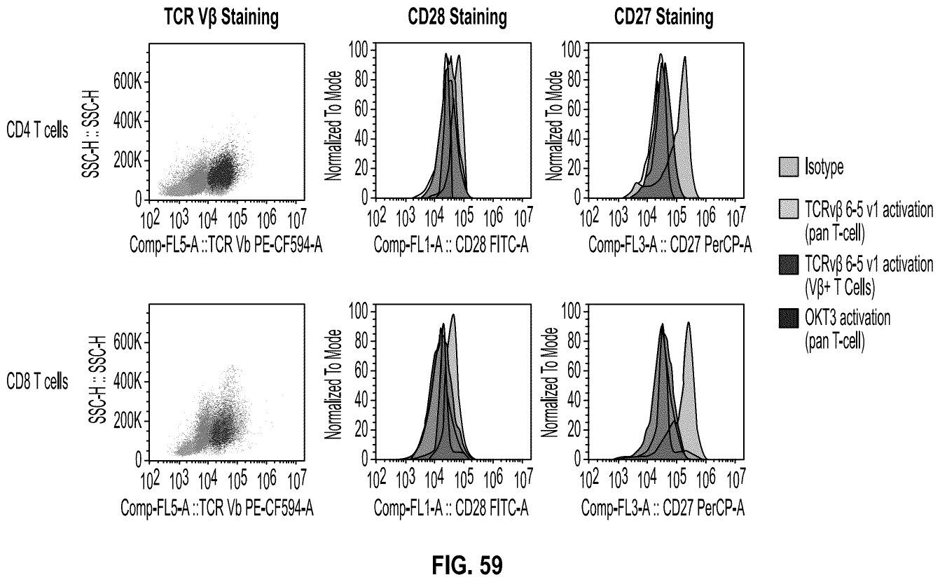

Further analysis of CD57 expression showed anti-TCR Vβ activated CD8+ T cells (FIG. 58A) do not display increased CD57 expression relative to anti-CD3ε activated CD8+ T cells (FIG. 58B). Likewise, analysis of CD27 and CD28 expression showed anti-TCR Vβ activated CD4+ T cells (FIG. 59 top) and anti-TCR Vβ activated CD8+ T cells (FIG. 59 bottom) do not display increased CD27 and CD28 expression relative to anti-CD3ε activated CD8+ T cells (FIG. 59).

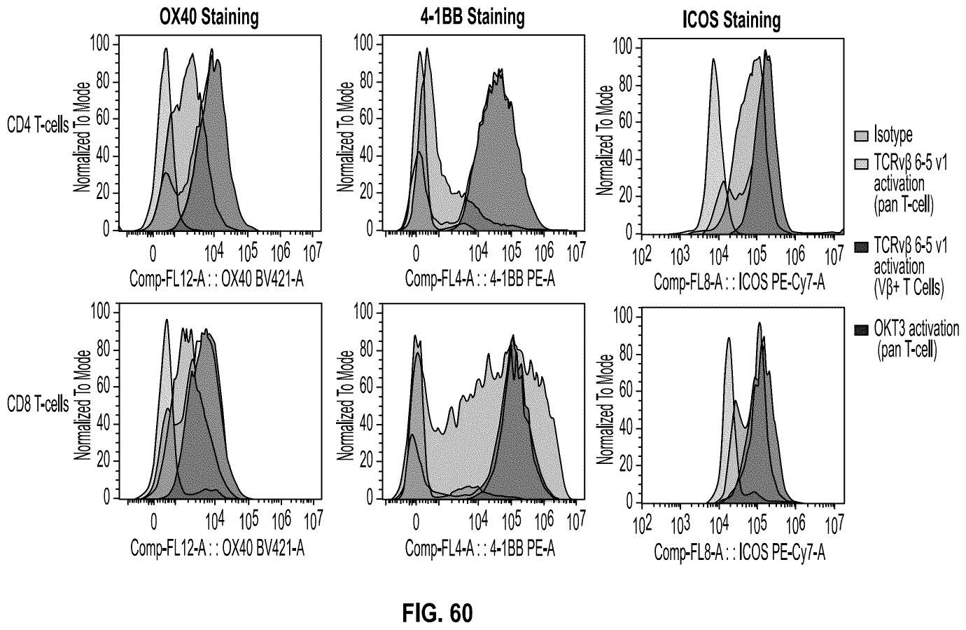

Further analysis of OX40, 41BB, and ICOS expression showed anti-TCR Vβ activated CD4+ T cells (FIG. 60 top) and anti-TCR Vβ activated CD8+ T cells (FIG. 60 bottom) display increased OX40, 41BB, and ICOS expression relative to anti-CD3ε activated CD8+ T cells (FIG. 60).

The TEMRA like phenotype of anti-TCR Vβ antibody expanded T cells was further analyzed using time lapse flow cytometry to evaluate expression of CD45RA and CCR7 at different time points post activation. Isolated human T-cells were activated with immobilized (plate-coated) anti-CD3ε or anti-TCR Vβ at 100 nM for between 1-8-days. After each (1, 2, 3, 4, 5, 6, 8-) day activation, T-cell subsets were identified by FACS staining for surface markers for Naïve/TSCM T cell (CD4+/CD8+, CD45RA+, CCR7+), T central memory (TCM; CD4+/CD8+, CD95+, CD45RA−, CCR7+), T effector memory (TEM; CD4+/CD8+, CD95+, CD45RA−, CCR7−), and T effector memory re-expressing CD45RA (TEMRA; CD4+/CD8+, CD95+, CD45RA+, CCR7−). TCRβV+ T-cells are identified by TCR Vβ+ staining. FACS stained samples were analyzed by flow cytometry analysis. Data shown a representative for CD4+ T-cells from 1 of 3 donors.

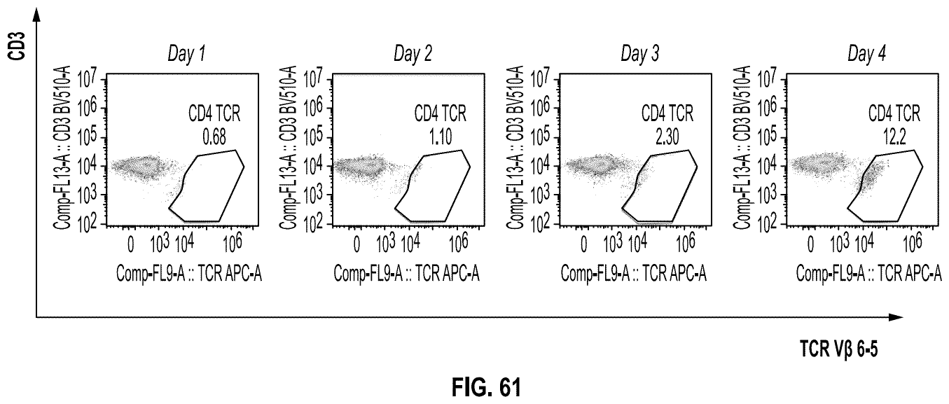

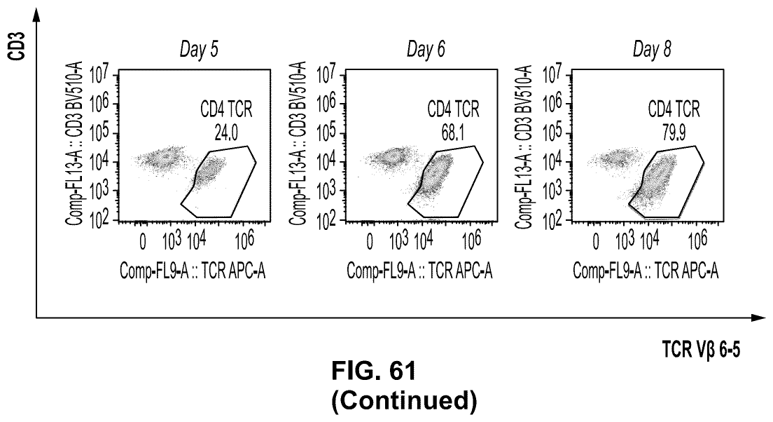

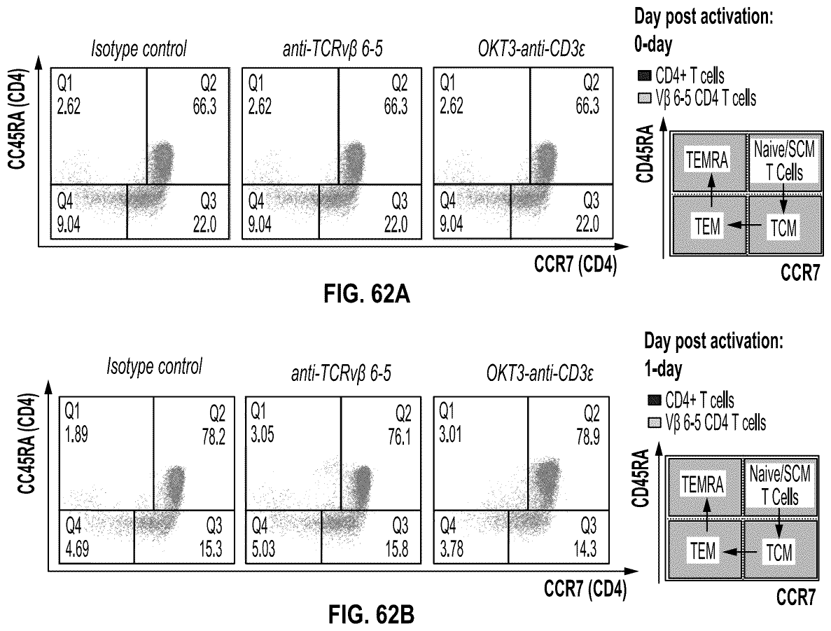

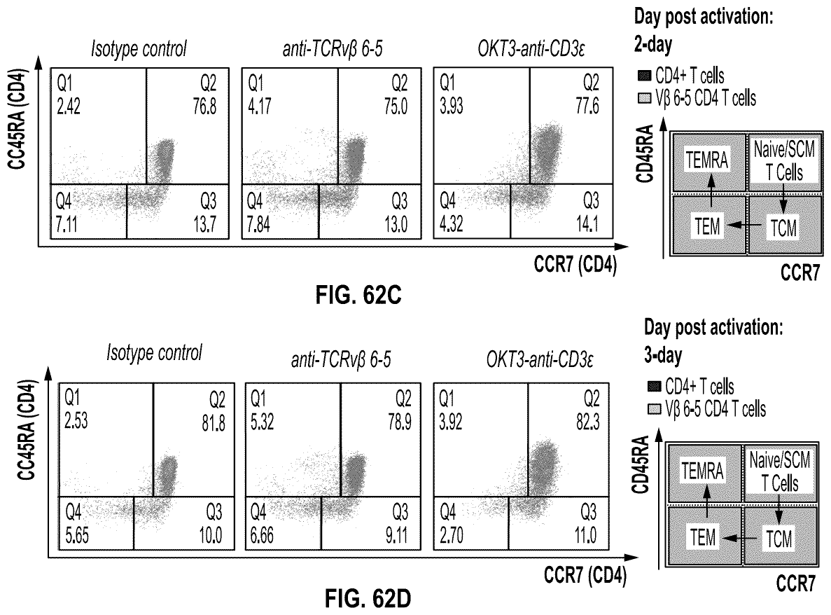

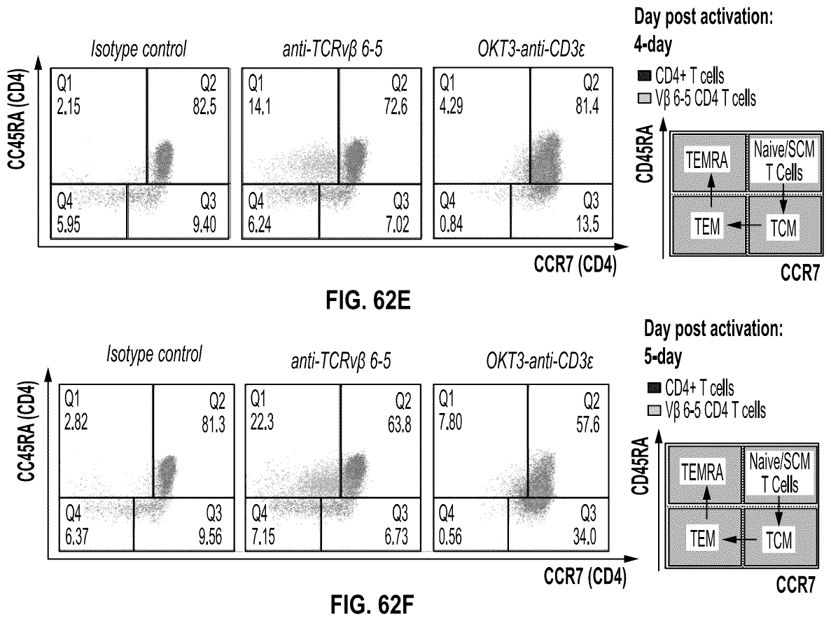

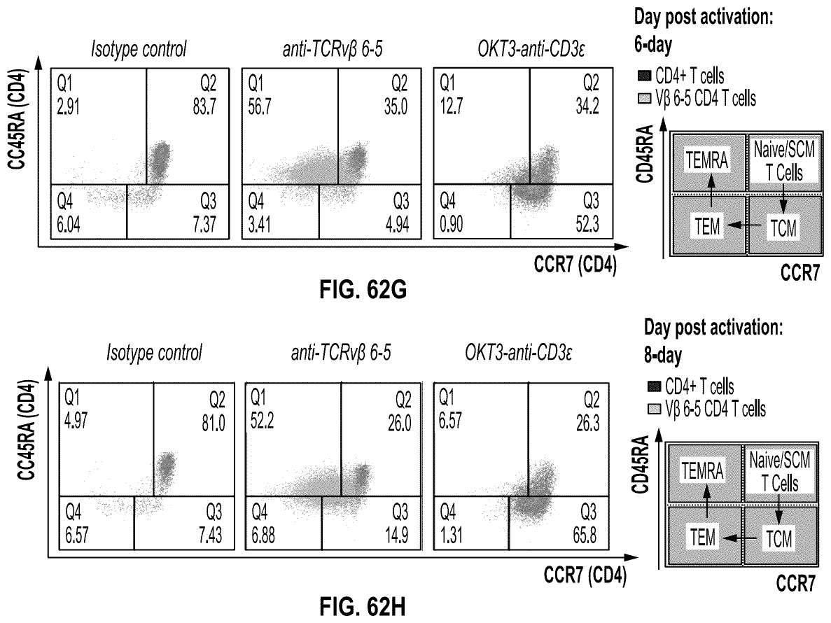

FIG. 61 shows a series of FACS plots showing the percentage of CD3+ (CD4 gated) TCRβV 6-5+ T cells 1, 2, 3, 4, 5, 6, and 8 days port activation with BCMA and the anti-TCR Vβ antibody anti-TCR Vβ 6-5 v1. Analysis of the percentage of CD4+ T cells expanded using isotype control (IgG1 N297A), anti-TCRβV (anti-TCR Vβ 6-5 v1), or anti-CD3ε (OKT3) antibodies on day 0 post activation (FIG. 62A), day 1 post activation (FIG. 62B), day 2 post activation (FIG. 62C), day 3 post activation (FIG. 62D), day 4 post activation (FIG. 62E), day 5 post activation (FIG. 62F), day 6 post activation (FIG. 62G), and day 8 post activation (FIG. 62H). The percentage of TEMRA like T cells expressing both CD45RA and CCR7 shows an increase in the population of TEMRA like cells in the CD4+ TCR Vβ 6-5+ T cell cultures expanded with the anti-TCR Vβ 6-5 v1 antibody compared to those expanded with the OKT3 antibody. Similar results were seen with CD8+ T cells. The results further show that purified human T-cells activated by anti-TCRβV 6-5 directly differentiates to TEMRA subsets and proliferate when compared to purified T-cells activated by anti-CD3ε (OKT3).

In summary, the data shows anti-TCRβV antibodies activated and expanded T cells represent a novel subset of recently activated effector T cells which share phenotypic markers with T_{EMRA}. This is in contrast to anti-CD3e-expanded T cells which differentiated into T_{CM} and T_{EM}, TCRβV expanded T cells are highly proliferative and do not upregulate the senescent marker CD57 OX40, 4-1BB, and ICOS are upregulated on anti-TCRβV activated T cells.

### Example 24: Metabolic State of αTCRβV Activated T Cells

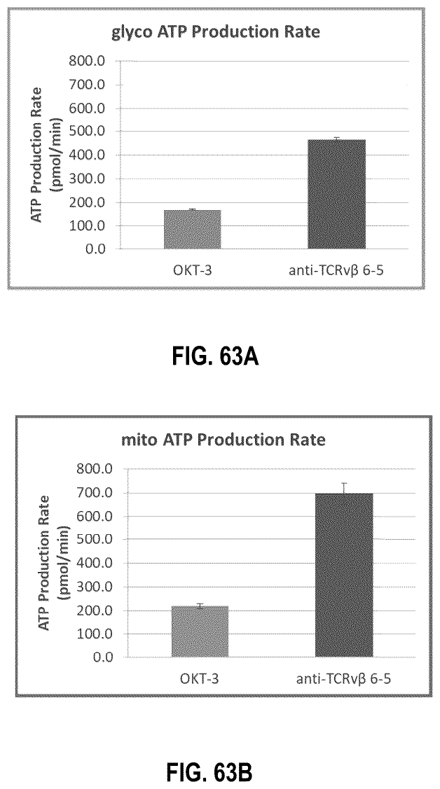

To evaluate the metabolic phenotype of T cells activated with αTCRβV antibodies-naïve T cells from PBMCs were stimulated and expanded for 5 days with plate-bound anti-CD3 antibody (OKT3) or anti-TCRβV antibody (anti-TCRβV 6-5 v1 antibody). Activated T cells were then rested in IL-2 containing media for 2 days, before they were cryopreserved. Prior to assay setup, cells were thawed and re-stimulated for 3 days with plate-bound anti-CD3 Ab (clone OKT3) or anti-TCRβV antibody (anti-TCRβV 6-5 v1 antibody), respectively. Equal numbers of live cells were plated on a Seahorse cartridge, and the Real-Time ATP Rate Assay was performed according to manufacturer's instructions. The data showed that ATP production from glycolysis (FIG. 63A) oxidative phosphorylation (FIG. 63B) in T cells from 3 donors (representative results from a single donor presented in FIG. 63A-67B) activated with the anti-TCRβV 6-5 v1 antibody increased compared to T cells activated with the OKT3 antibody (3-fold increase in ATP production was observed on average); and one donor showed equal levels of ATP production in anti-TCRβV 6-5 v1 and OKT3 Ab stimulated cells (data not shown).

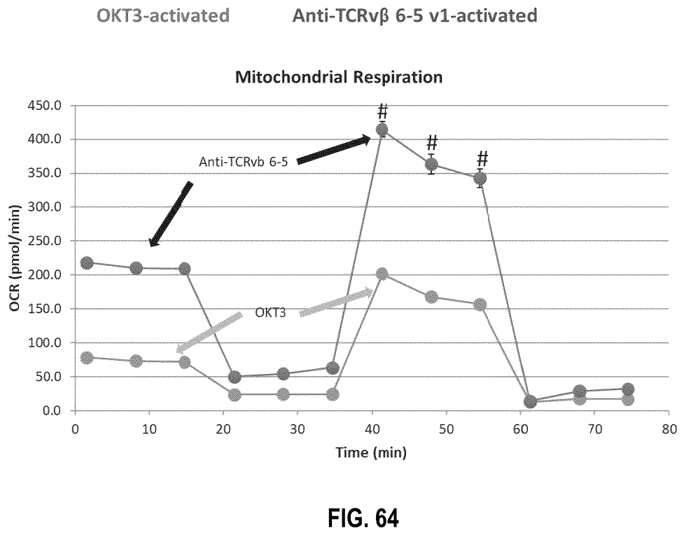

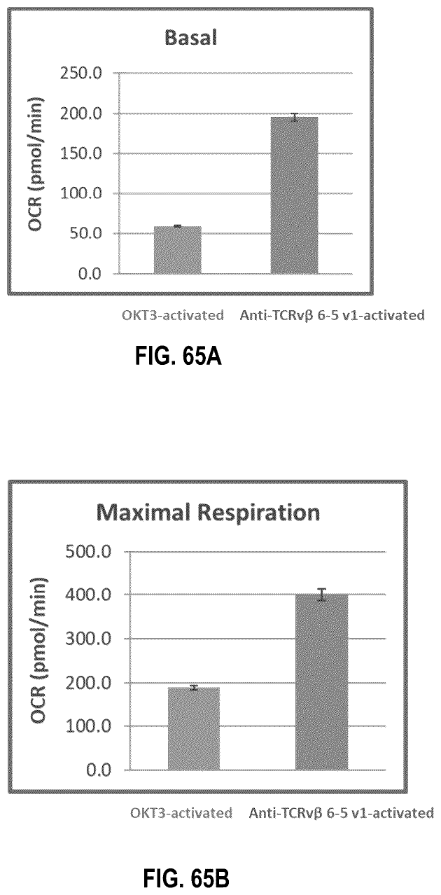

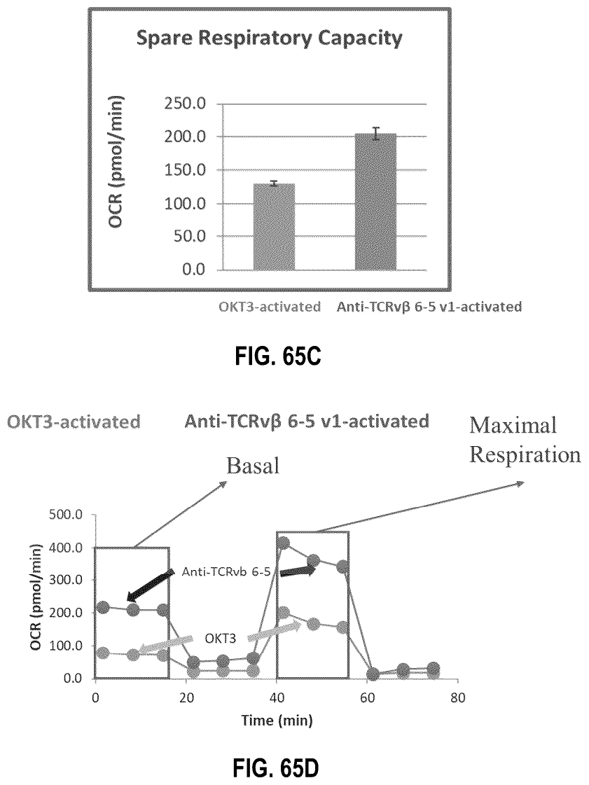

The increased mitochondrial respiration in T cells activated with anti-TCRβV 6-5 v1 antibody compared to T cells activated with the OKT3 antibody is further shown in FIG. 64, which shows the oxygen consumption rate (OCR) of T cells from about 0 to 75 minutes activated with the indicated antibody. Data in FIG. 63 is from a single donor; a second donor tested showed equal levels of ATP production in anti-TCRβV 6-5 v1 and OKT3 Ab stimulated cells (data not shown). FIGS. 65A-65C show the oxygen consumption rate (OCR) of T cells activated with the indicated antibody during basal respiration (FIG. 65A), maximal respiration (FIG. 65B), and spare respiratory capacity (FIG. 65C). Cells were plated in media containing glucose and glutamine to measure basal OCR. FCCP (ETC accelerator) was added to the cell culture medium to determine maximum respiratory capacity/max OCR. Antimycin A & Rotenone (ETC inhibitor) were added to the cell culture medium to determine spare respiratory capacity and non-mitochondrial oxygen consumption. The data presented in FIGS. 65A-65C α-TCRβV 6-5 v1 activated T cells had significantly increased basal respiration, maximal respiration, and spare respiratory capacity compared to α-CD3 (OKT3) activated T cells (data from a single donor). A second donor was tested which showed equal levels of ATP production in anti-TCRβV 6-5 v1 and OKT3 Ab stimulated cells (data not shown).FIG. 65D indicates the areas of basal respiration and maximal respiration as shown in FIG. 64A and FIG. 64B, respectively.

In order to determine if the observed increase in metabolism due to differences in T cell stimulation, or is it intrinsic to the differentiation stage of T cells activated with anti-TCRβV antibodies TCRβV 6-5+ T cells were expanded for 5 days with plate-bound anti-TCRβV 6-5 vI Ab. Cells were then rested in IL-2 containing media for 2 days and cryopreserved. Upon thawing, cells were re-stimulated with anti-TCRβV 6-5 v1 for 3 days. Cells were then counted and equal numbers of live cells were re-seeded and stimulated with plate-bound anti-CD3 Ab (clone OKT3) or anti-TCRβV 6-5 v1, respectively, for 24 hours. Equal numbers of live cells were plated on the Seahorse cartridge and the Real-Time ATP Rate Assay was performed.

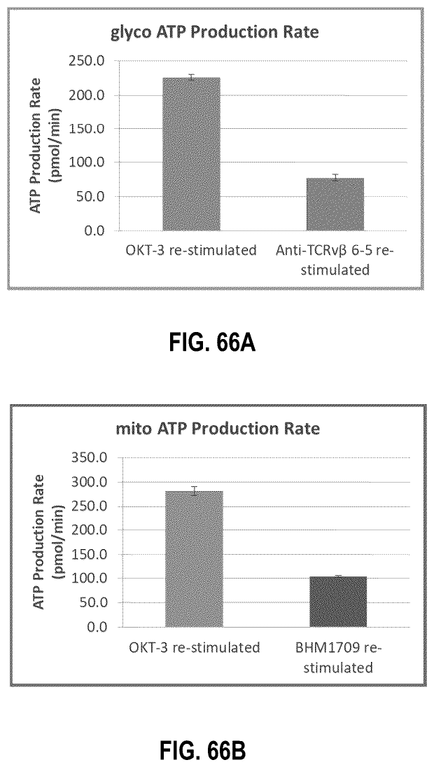

The results show that ATP production by glycolysis (FIG. 66A) and oxidative phosphorylation (FIG. 66B) by T cells activated with anti-TCRβV 6-5 v1 is significantly increased upon re-stimulation with α-CD3 antibody OKT3 versus α-TCRβV 6-5 v1 antibody. The observed increase in metabolism of T cells activated with anti-TCRβV 6-5 v1 appears to be due to intrinsic differences upon differentiation into these cells. T cells activated with anti-TCRβV 6-5 v1 have an increased metabolism compared to CD3-activated T cells, which can be further enhanced with strong T cell stimulation via OKT3.

In summary, the results show that T cells activated with anti-TCRβV antibodies have a metabolic memory phenotype. The cells are not metabolically exhausted, because exhausted T cells have a decreased metabolism, α-TCRβV 6-5 v1-stimulation induces a T cell differentiation stage, which is highly metabolically active, indicative of an effector memory phenotype. This metabolic phenotype is maintained when these cells are re-stimulated with other T cell engagers (OKT3).

### Example 25: Assessment of CRS with Anti-CD3e Antibody Compared to Anti-TCRβV Antibody

To determine CRS effect of a low affinity (Teneobio) anti-CD3e antibody, a cytokine release assay (CRA) with PBMCs was used Briefly. PBMCs from two donors were stimulated with plate-coated antibodies; anti-TCRvb6-5 v2, anti-CD3e (SP34) or Teneobio's anti-CD3e antibody. T cell activating antibodies were tested at 100 nM, the highest concentration previously shown not to induce CRS cytokines in this assay. Supernatants were collected at day 1, 3, 5 and 7. Cytokine secretion measurement (IFN-g, IL-10, IL-15, IL-17A, IL-1a, IL-1b, IL-2, IL-4, IL-6 and TNF-a) was detected using MSD analysis. The data show results from two donors.

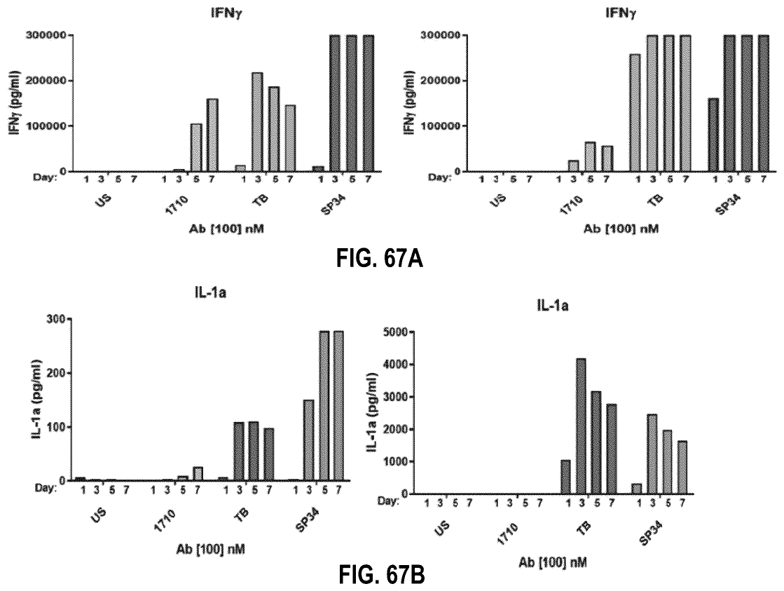

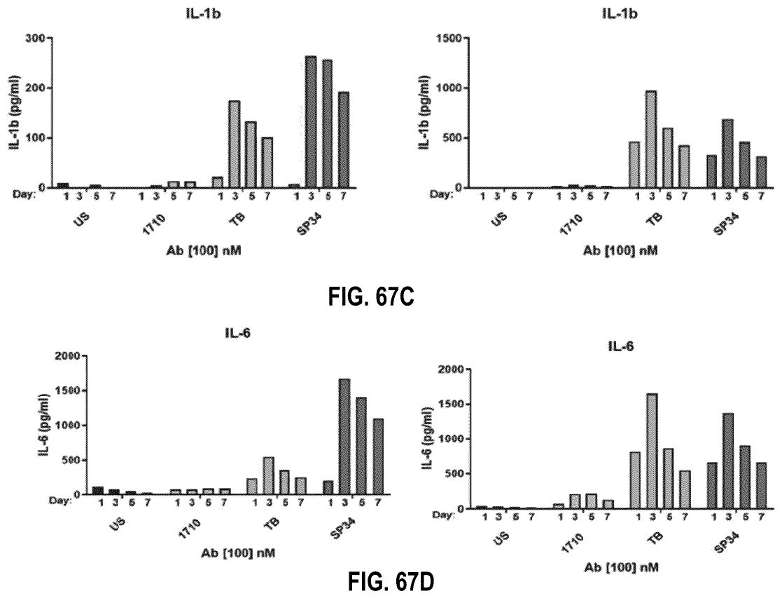

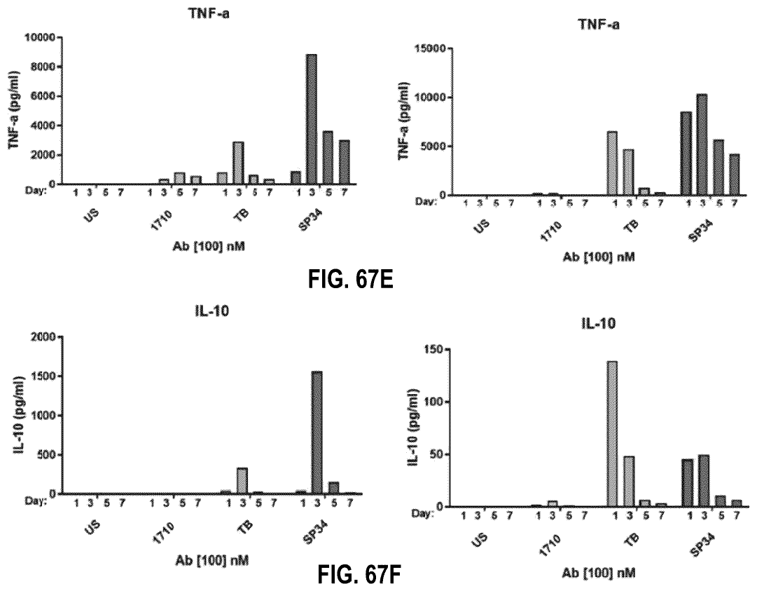

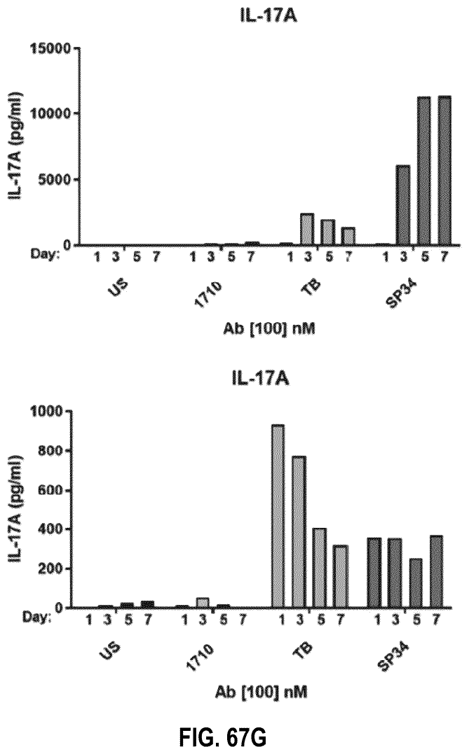

FIGS. 67A-67F show that a reduced affinity anti CD3e antibody (TeneoBio) induces expression of IFNgamma, TNFalpha, IL-1a, IL-1b, IL-6 (CRS and neurotoxicity associated cytokines) similar to the SP34-2 anti CD3e antibody. In contrast, the anti-TCRvb6-5 v2 as described herein does not induce CRS or neurotoxicity associated cytokines.

In summary the data shows that Tenebio's anti-CD3e antibody induces cytokines associated with CRS and neurotoxicty in this highly sensitive PBMC CRA. Thus, Tenebio's anti-CD3e antibody has potential to induce CRS and NT as seen with SP34-based T cell-redirecting bispecific molecules. The anti-TCRvb6-5 as described herein does not induce CRS- and NT-associated cytokines in this assay, suggesting that in some embodiments, TCRvb6-5 based antibodies may be amenable to administration at higher doses and avoid MABEL (minimum anticipated biologic effect level) dosing regimen required for current CD3e-based bispecific molecules.

### Example 26: Anti-TCRβV Stimulated PBMC Mediated Stimulation of NK Cell Expansion

To assess whether anti-TCRβV stimulated PBMCs mediate expansion of NK cells in vitro-human PBMCs were stimulated with 100 nM of plate-coated anti-TCRβV 6-5 v1 anti-CD3ε (OKT3 and SP34-2) for up to 7 days. NK cells were identified via FACS staining for CD3−/CD56+/CD16+/NKp46+ populations. NK cell count was determined by a constant μl sample (presented as relative count for each donor). NK cell-mediated target cell lysis was determined 6-days post stimulation, in which PBMCs were harvested and co-cultured with K562 target cells for 4 hours to determine cell killing, via DRAQ7 viability FACS staining.

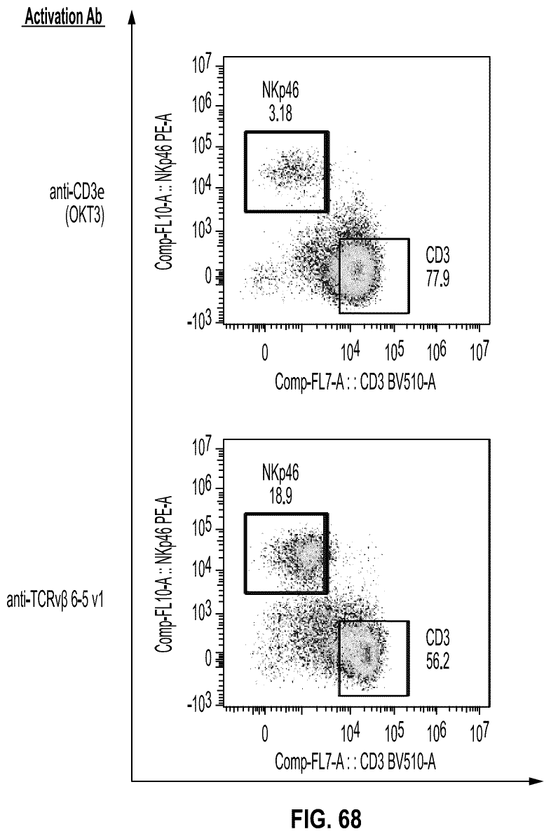

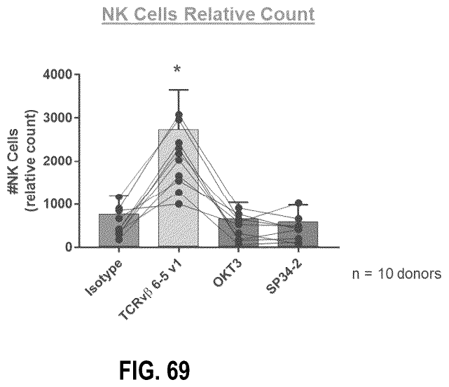

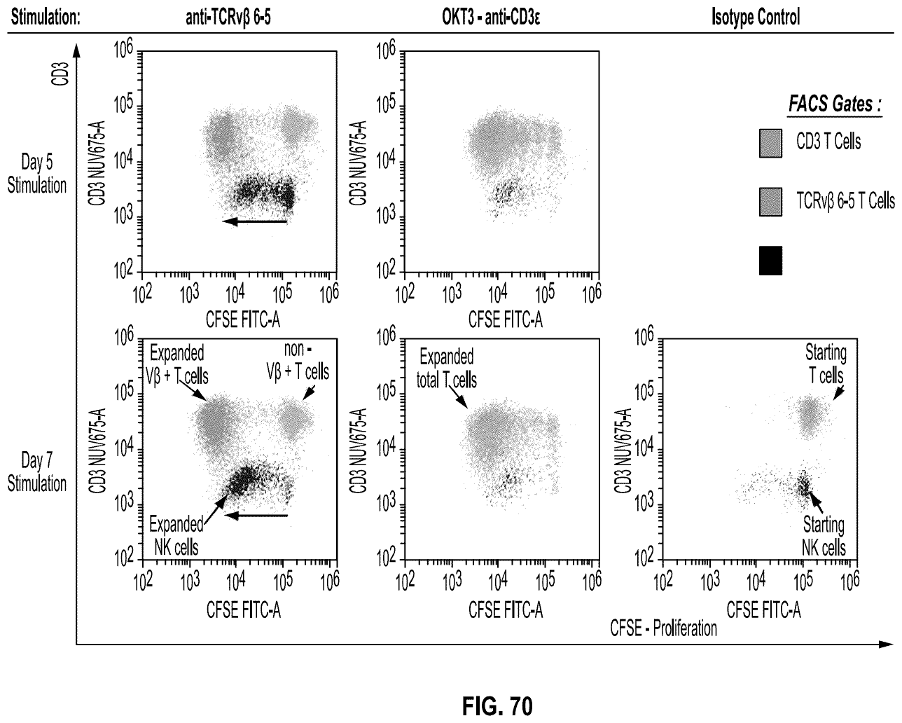

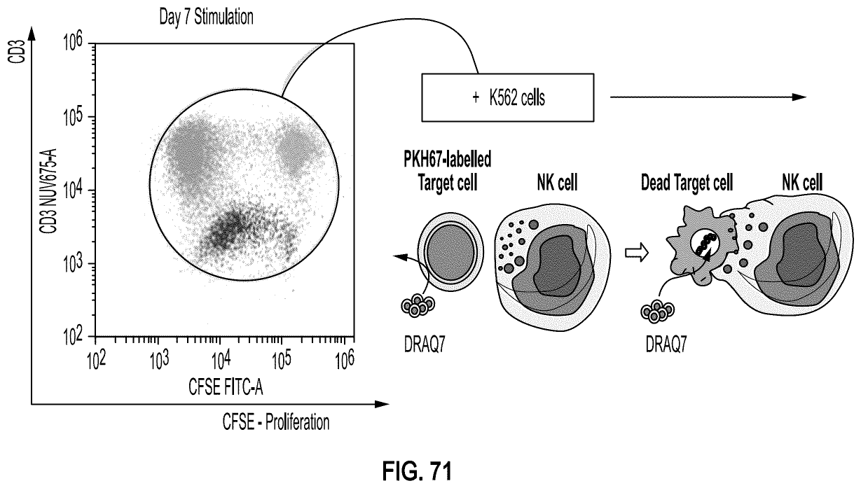

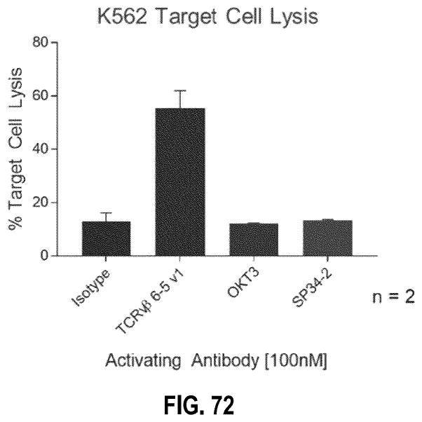

The results show that anti-TCRβV stimulation increases NK cell numbers compared to OKT3 stimulation (FIG. 68; FIG. 69). FACS CFSE staining further shows NK cell proliferation (FIG. 70). FIG. 71 and FIG. 72 show NK cell mediated lysis of target K562 cells. In summary, anti-TCRβV 6-5 antibody induces expansion of NK cells in PBMC; and this effect is unlikely to be mediated through the FcR on NK cells as anti-CD3ε antibodies did not expand NK cells. Expanded NK cells by anti-TCRβV 6-5 v1 mediate potent target cell (K562) lysis in vitro.

In addition to the experiments conducted above using the anti-TCRβV 6-5 v1 antibody, similar experiments were carried out using anti-TCRβV antibodies that recognize different clonotypes. In one experiment, the anti-TCRβV 12 antibodies; anti-TCRvβ 12-3/4 v1, anti-TCRvβ 12-3/4 v2, and anti-TCRvβ 12-3/4 v3 were used to activate/expand PBMCs using solid-phase stimulated (plate-coated) with the indicated T cell-activating antibody at 100 nM for 6 days as described above. Flow analysis was performed for NK cells using NKp46 and CD56 (CD3 negative). Data was generated from 3 donors and representative of 1 independent experiments.

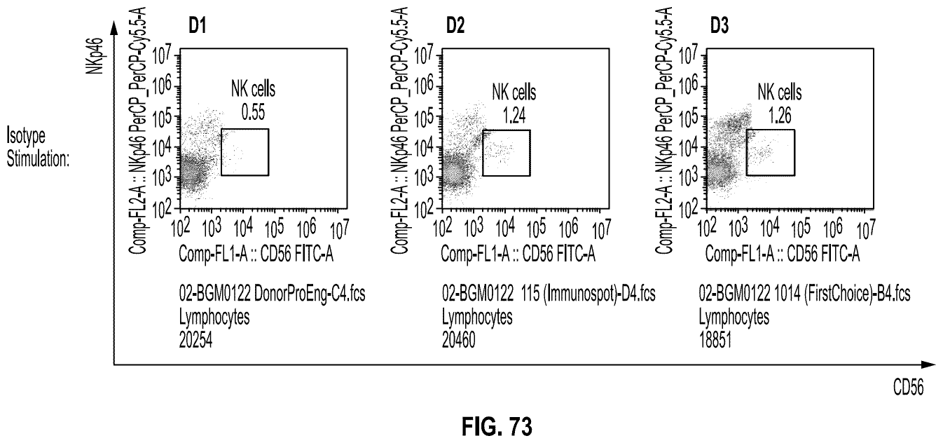

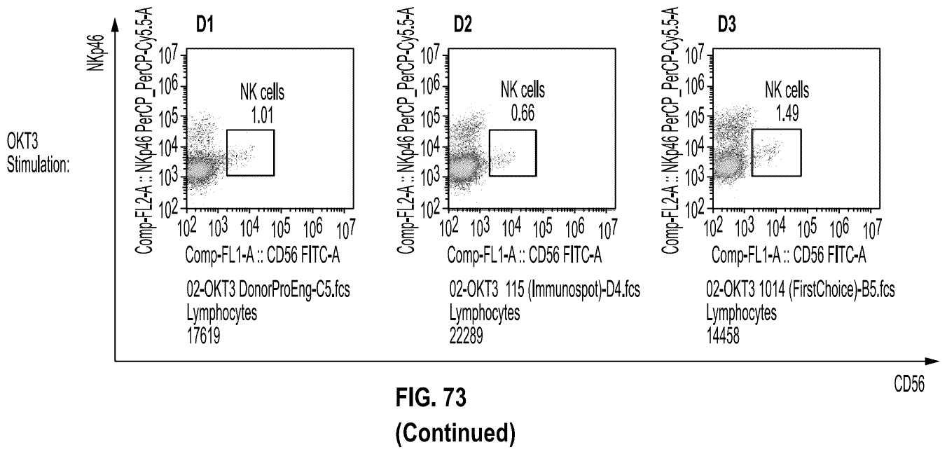

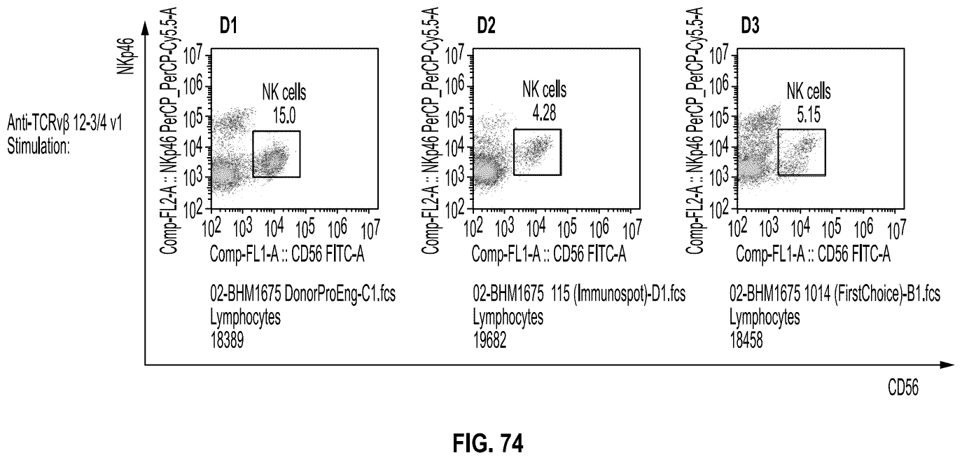

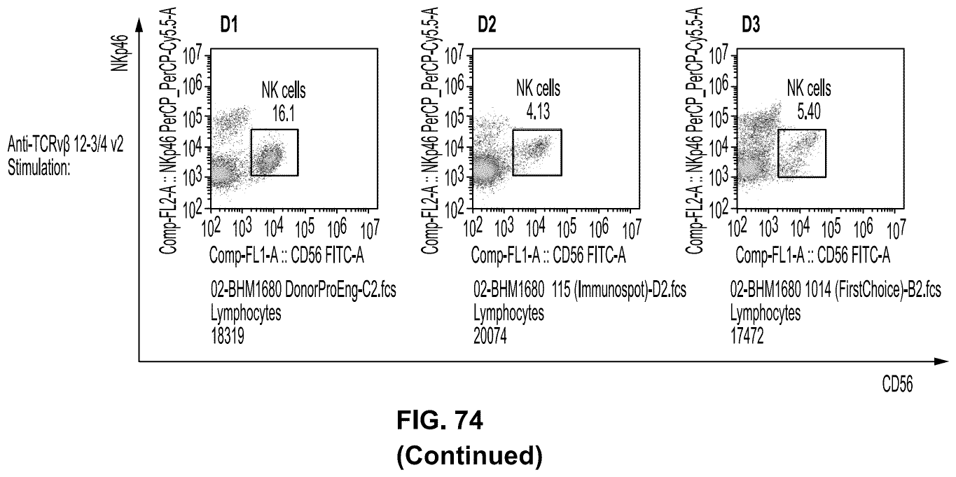

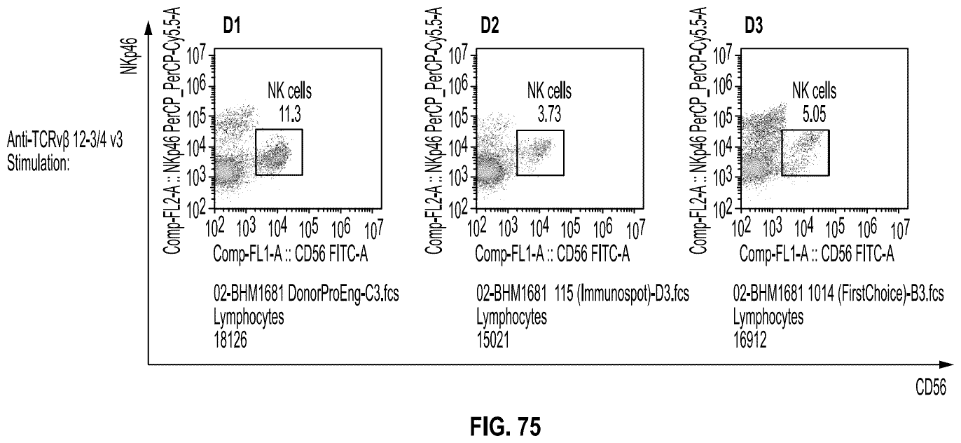

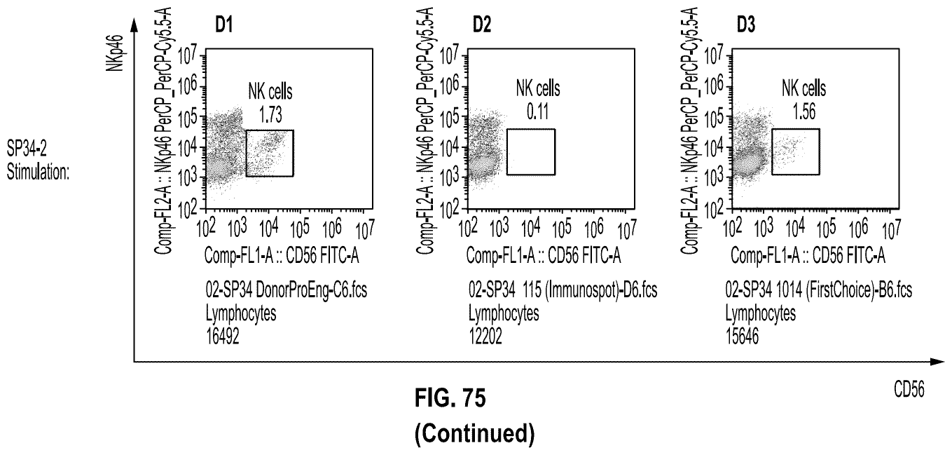

Activation/expansion of the PBMC's with isotype control or the anti-CD3ε antibody OKT3 or SP34-2 did not induce expansion of NK cells (FIG. 73: FIG. 75). However, activation/expansion of PBMCs with anti-TCRvβ 12-3/4 v1 (FIG. 74), anti-TCRvβ 12-3/4 v2 (FIG. 74), and anti-TCRvβ 12-3/4 v3 (FIG. 75) all induced NK cell expansion. In summary, the data shows that anti-TCRvb 12 antibodies are able to induce indirect expansion of NK cells from PBMC cultures in vitro.

### Example 27: Concentration Response to Anti-TCRβV Stimulation In Vitro

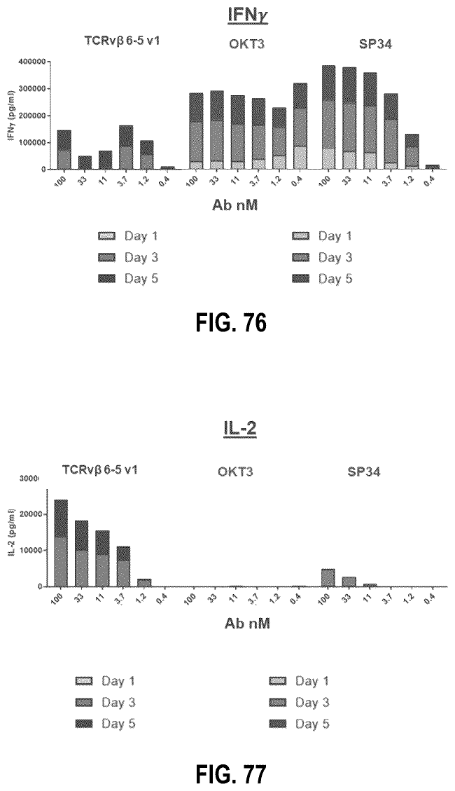

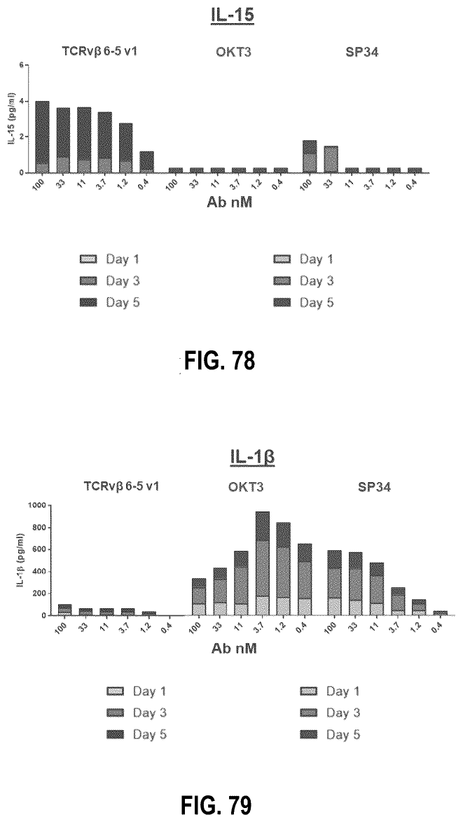

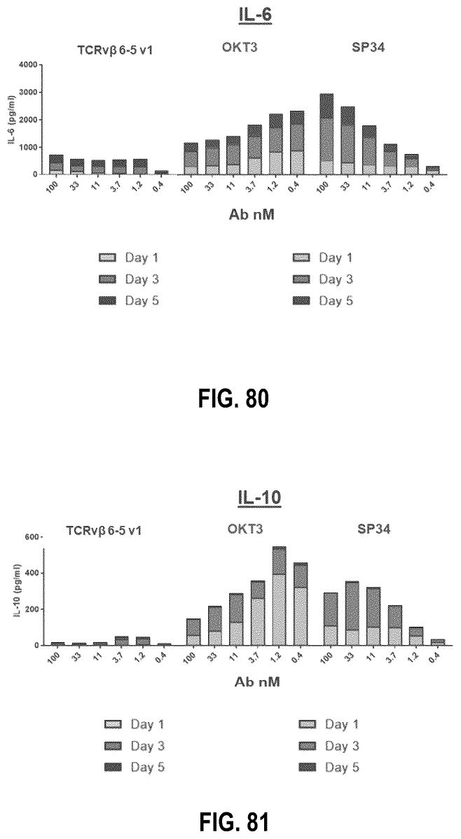

Human PBMCs were solid-phase stimulated (plate-coated) with the indicated T cell-activating antibody at the indicated different concentrations: i) anti-TCRvb 6-5 v1 antibody: ii) OKT3 (anti-CD3a antibody); or iii) SP34-2 (anti-CD3ε antibody). Supernatant were collected on day 1, day 3 and day 5 and cytokines quantified by using Meso Scale Discovery (MSD) assay. The production of cytokines IFNγ (FIG. 76), IL-2 (FIG. 77), IL-15 (FIG. 78), IL-1β (FIG. 79), IL-6 (FIG. 80), and IL-10 (FIG. 81) was analyzed. The results indicate that the lack of CRS associated cytokine induction by T cells activated with an anti-TCRvb is not a result of inhibition or toxicity due to high antibody concentrations.

### Example 28: T Cells Activated by Anti-TCRβV Antibodies have a Distinct Cytokine Release Profile Compared to T Cell Activated with Anti-CD3 Antibodies

To assess the cytokine release profile of T cells activated/expanded using anti-TCRβV antibodies as compared to anti-CD3ε antibodies-PBMCs were cultured in cell culture plates coated with the immobilized anti-TCRβV antibody anti-TCRβV 6-5 v1 or an anti-CD3ε antibody, either OKT3 or SP37-2. The cells were cultured for 1-8 days, the supernatant collected, and cytokines analyzed using Meso Scale Discovery (MSD) assay. T cells samples from numerous different human donors were tested.

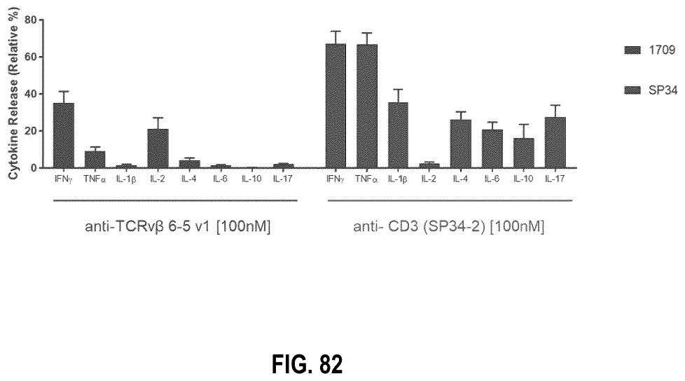

FIG. 82 shows a summary of data from 17 donors. The highest overall cytokine secretion from time points (day 3 and beyond) was used for further analysis. Each data point was normalized against the highest secretion for each donor and showed as relative % of highest (at a confidence interval of 0.95 percentile). The data shows that T cells activated/expanded with an anti-TCRβV antibody as compared to anti-CD3ε antibody release less IFNγ, TNFα, IL-1β, IL-4, IL-6, IL10, and IL-17; while releasing an increased amount of IL-2 (FIG. 82).

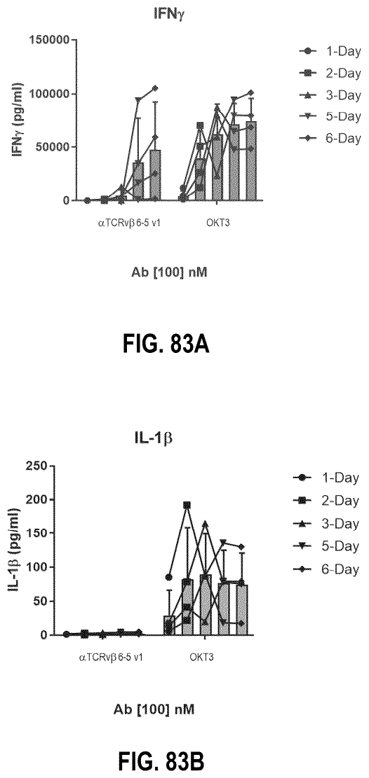

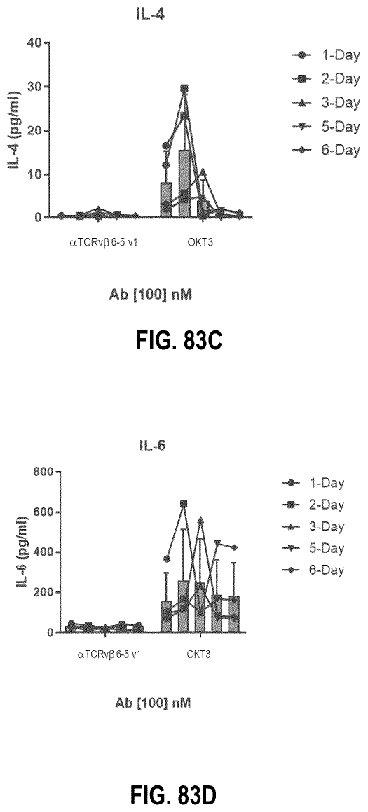

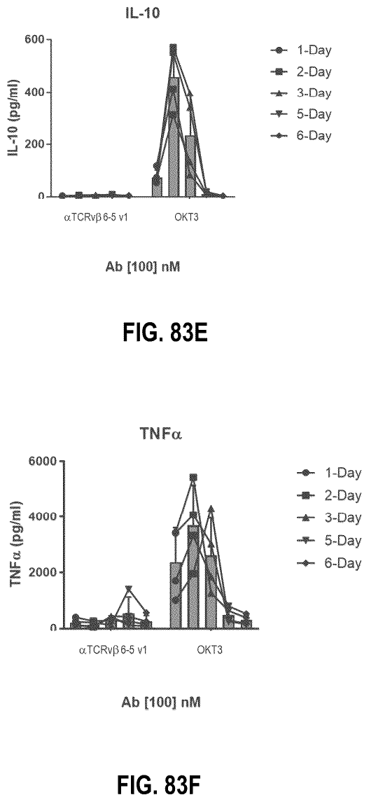

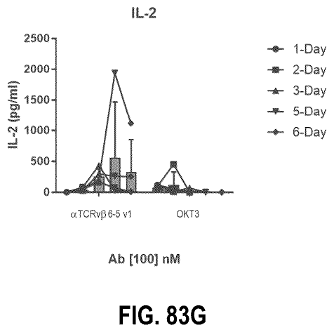

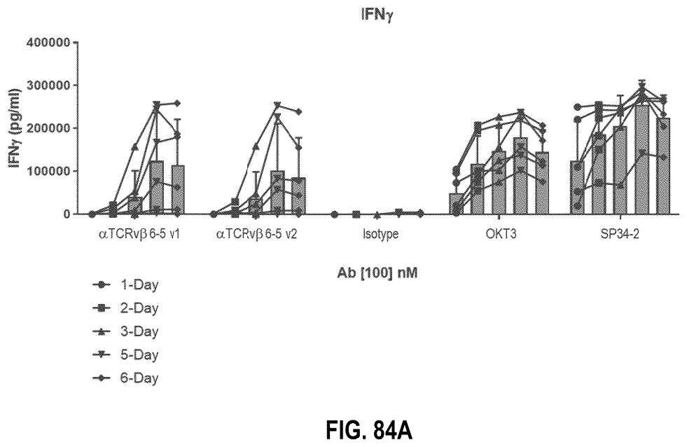

A series of experiments using the methods previously described, but varying the culture period were conducted with PBMCs from different donors. In one experiment, PBMCs from four different donors were cultured in plates coated with immobilized anti-TCRβV antibody anti-TCRβV 6-5 v1 or an anti-CD3ε antibody, either OKT3 or SP37-2 for 1-6 days. The data confirms that T cells activated/expanded with an anti-TCRβV antibody as compared to anti-CD3ε antibody release lower levels of IFNγ (FIG. 83A), IL-1β (FIG. 83B), IL-4 (FIG. 83C), IL-6 (FIG. 83D), IL10 (FIG. 83E), and TNFα (FIG. 83F); and higher levels of IL-2 (FIG. 83G).

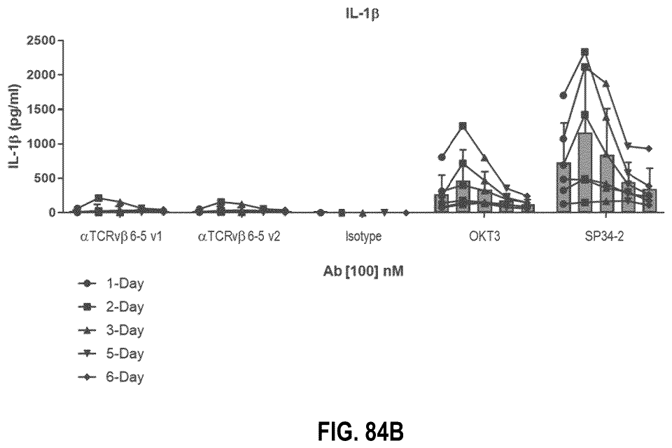

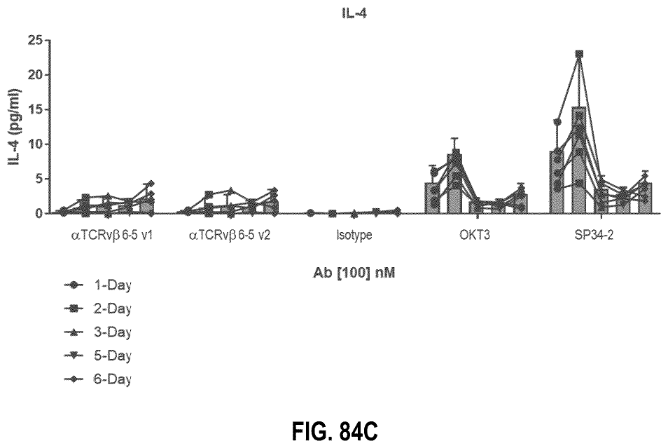

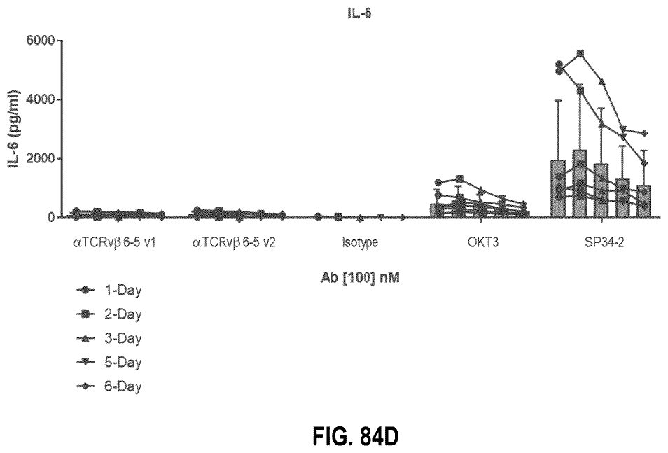

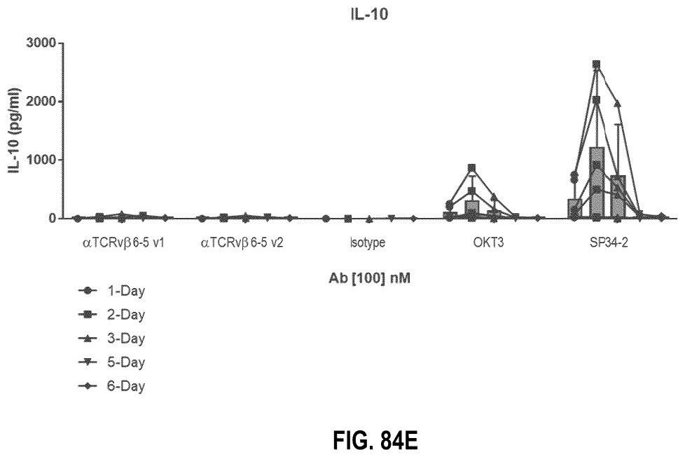

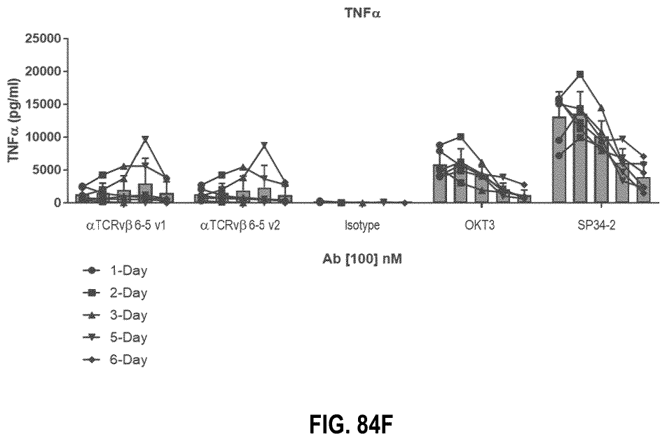

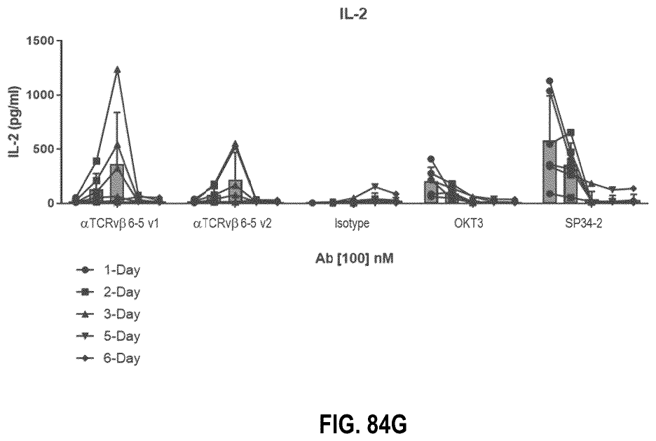

In a second experiment, PBMCs from six different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 1-6 days, or isotype control. The data confirms that T cells activated/expanded with an anti-TCRβV antibody as compared to anti-CD3ε antibody release lower levels of IFNγ (FIG. 84A), IL-1β (FIG. 84B), IL-4 (FIG. 84C), IL-6 (FIG. 84D), IL 10 (FIG. 84E), and TNFα (FIG. 84F); and higher levels of IL-2 (FIG. 84G).

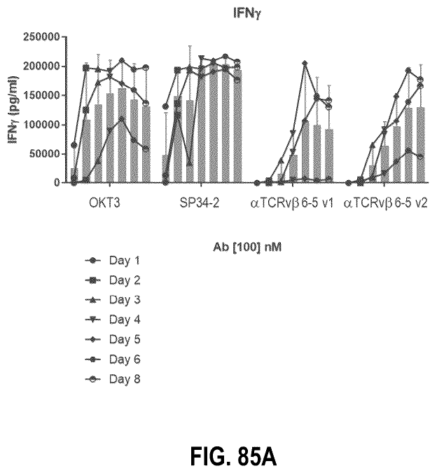

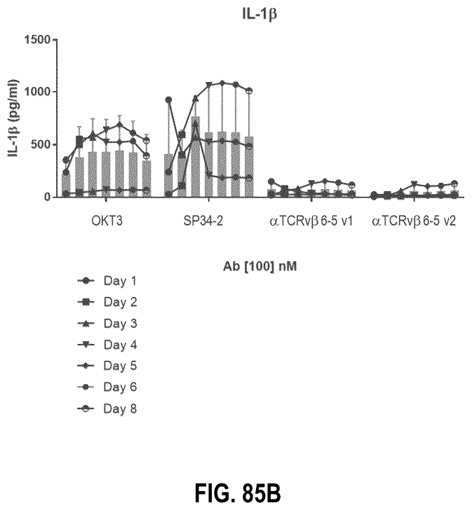

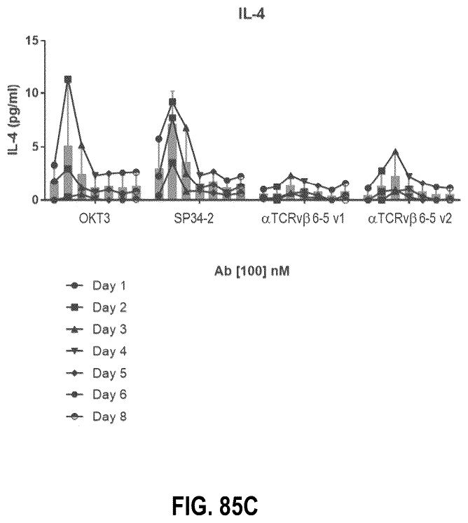

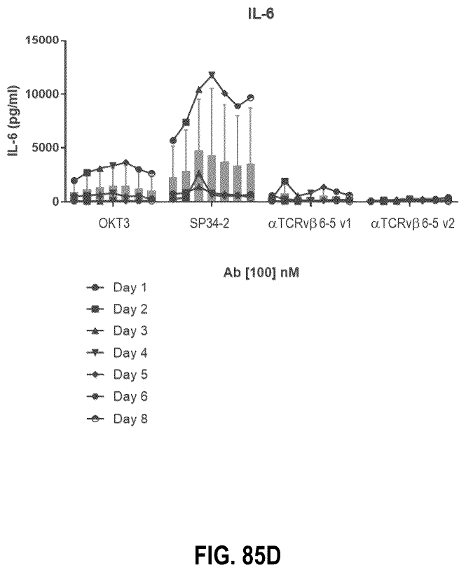

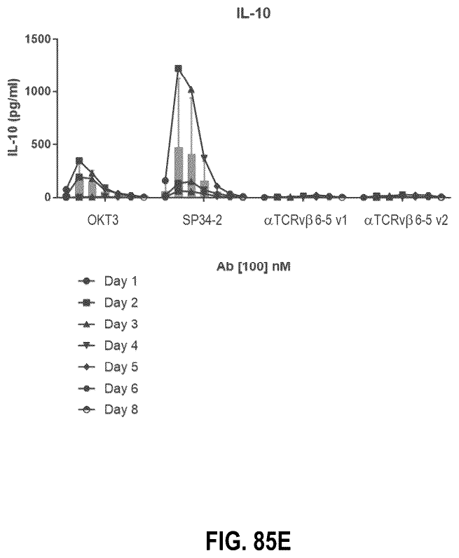

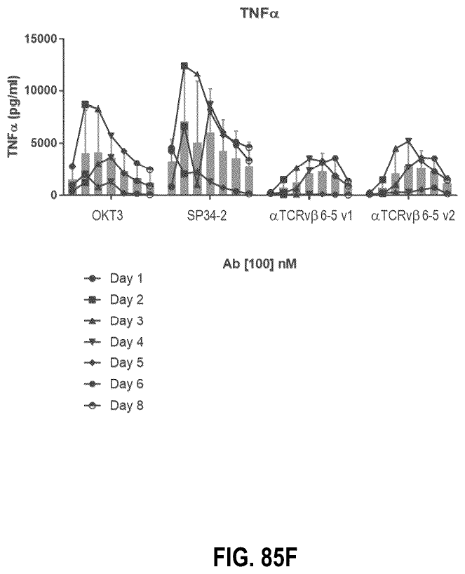

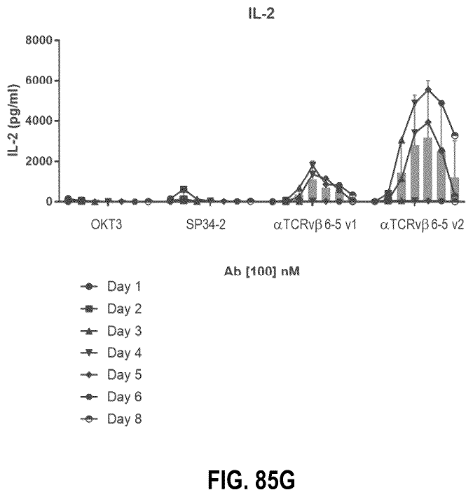

In a third experiments, PBMCs from three different donors were cultured in plates coated with immobilized anti-TCRβV antibody, either anti-TCRβV 6-5 v1 or anti-TCRβV 6-5 v1; or an anti-CD3E antibody, either OKT3 or SP37-2 for 1-8 days, or isotype control. The data confirms that T cells activated/expanded with an anti-TCRβV antibody as compared to anti-CD3ε antibody release lower levels of IFNγ (FIG. 85A), IL-1β (FIG. 85B), IL-4 (FIG. 85C), IL-6 (FIG. 85D), IL10 (FIG. 85E), and TNFα (FIG. 85F); and higher levels of IL-2 (FIG. 85G).

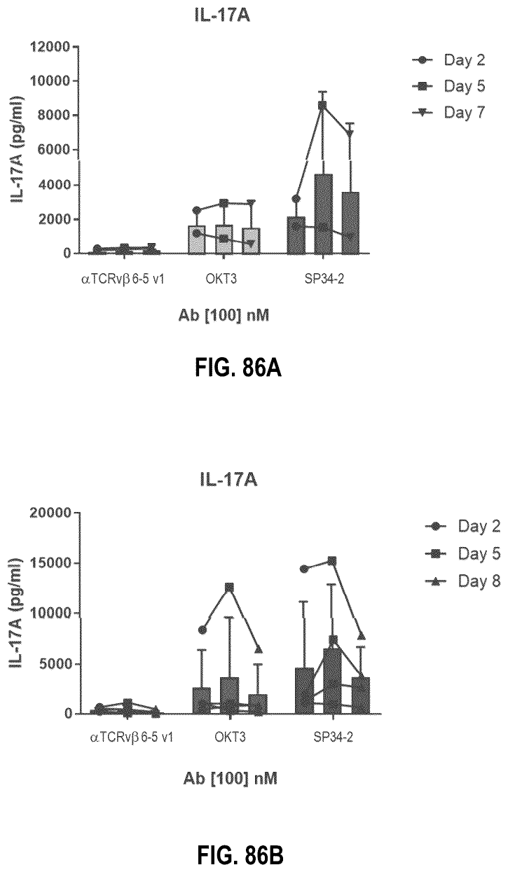

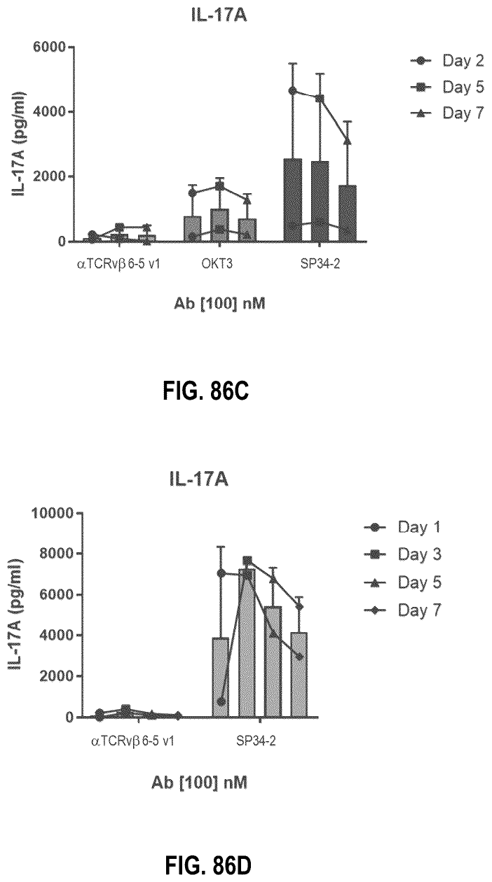

In a fourth experiments. PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 86A). In a fifth experiments, PBMCs from four different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-8 days, or isotype control. The data confirms that T cells activated/expanded with an anti-TCRβV antibody as compared to anti-CD3ε antibody release lower levels of IL-17A (FIG. 86B). In a sixth experiments, PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 86C). In a seventh experiments, PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 86D).

A series of similar experiments were conducted using the TCRβV antibody anti-TCRβV 6-5 v1 or anti-TCRvb 12-3/4 v1 to further assess the cytokine release profile of T cells activated/expanded using anti-TCRβV antibodies as compared to anti-CD3ε antibodies. As described above, PBMCs were cultured in cell culture plates coated with the immobilized anti-TCRβV antibody, anti-TCRβV 6-5 v1 or anti-TCRvb 12-3/4 v1; or an anti-CD3ε antibody, either OKT3 or SP37-2; isotype control; or anti-TCRβV 6-5 v1 in combination with. The cells were cultured for 1-8 days, the supernatant collected, and cytokines analyzed using Meso Scale Discovery (MSD) assay. Data generated from 2 donors and representative of 2 independent experiments.

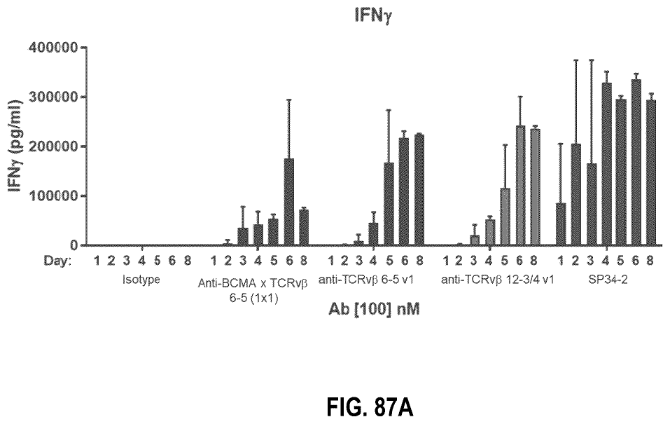

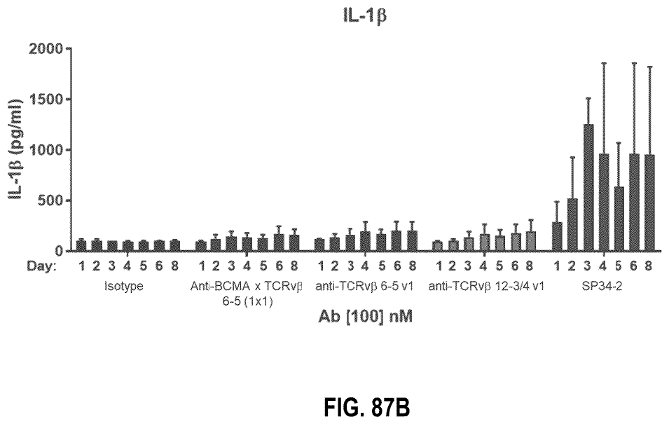

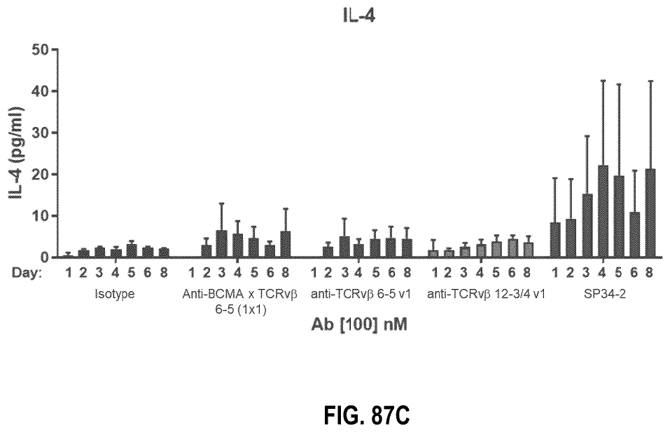

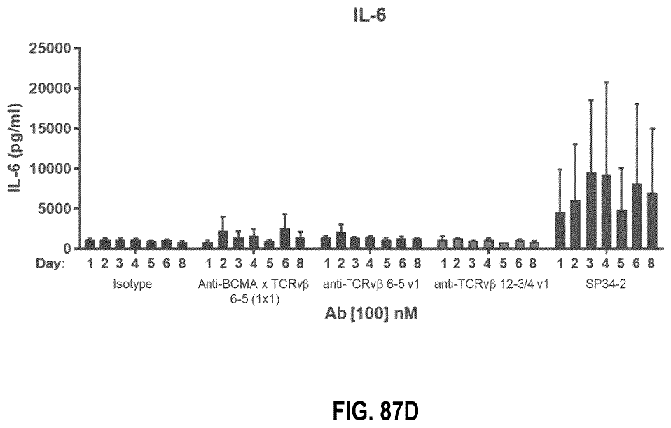

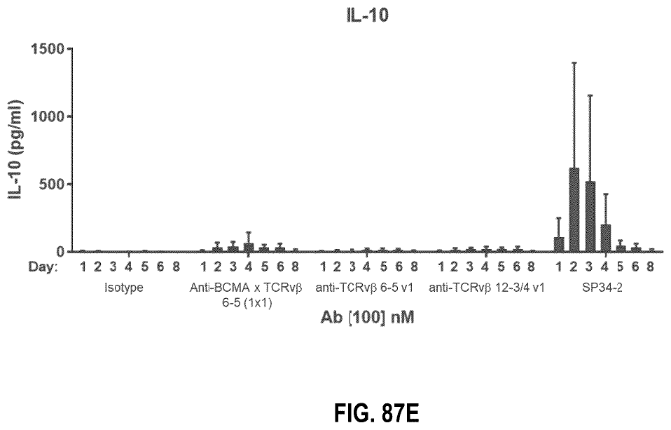

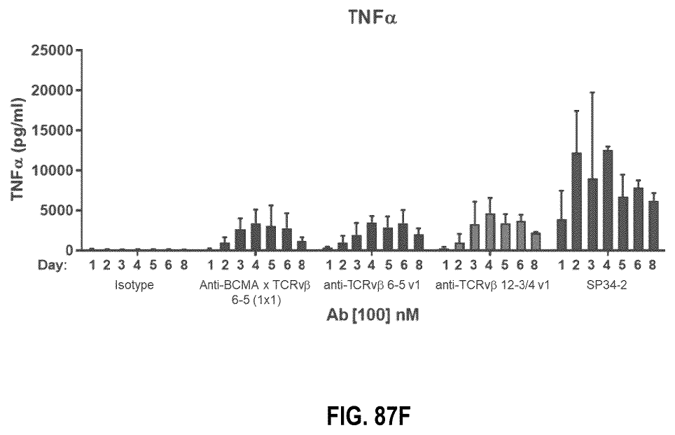

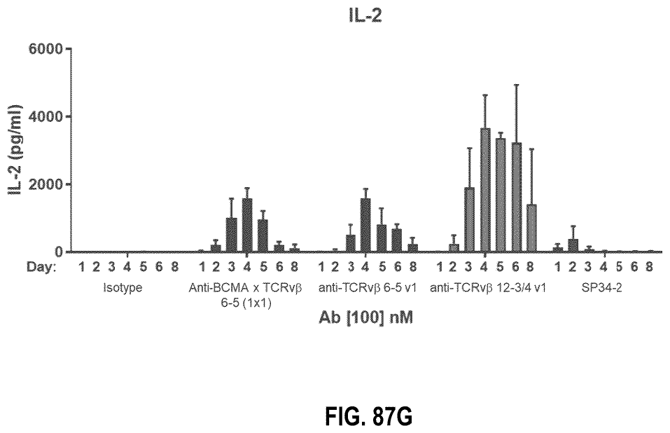

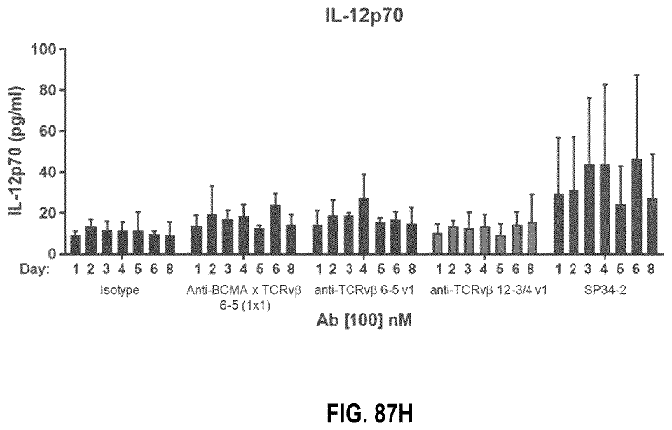

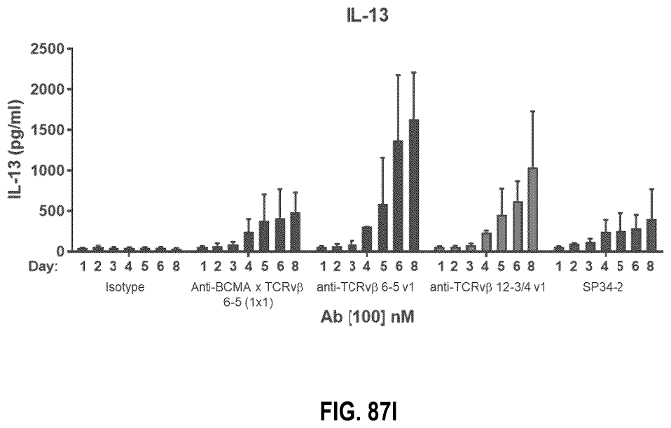

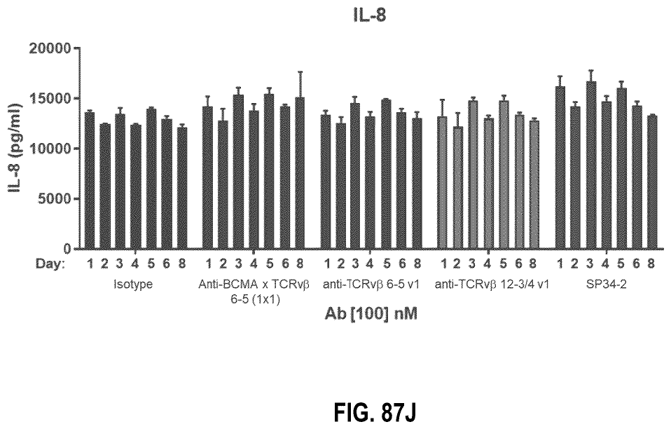

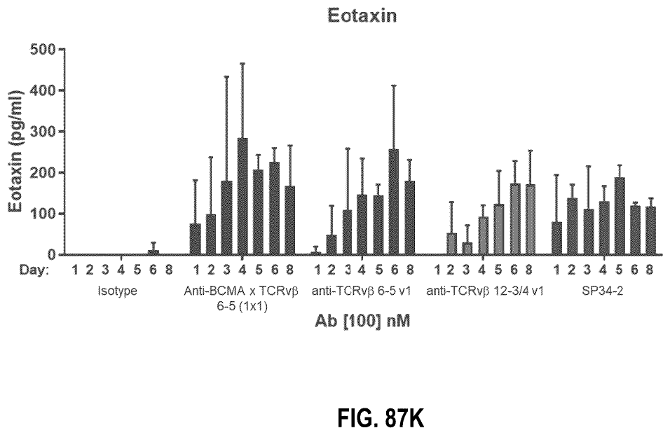

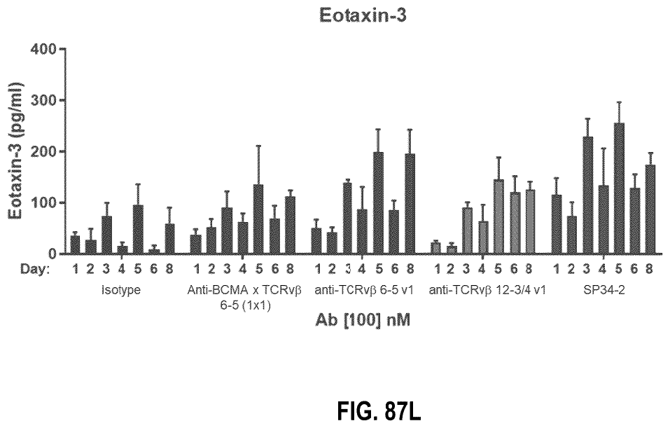

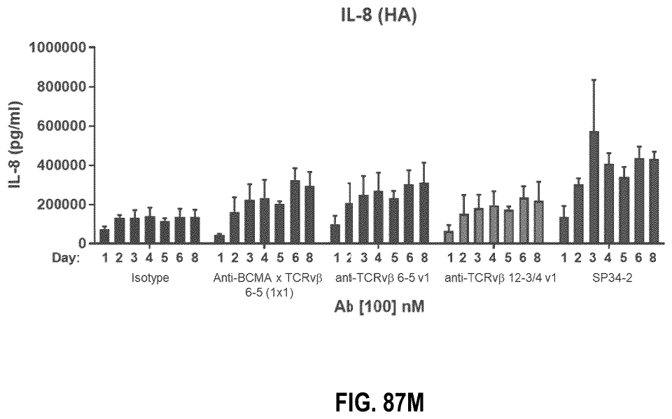

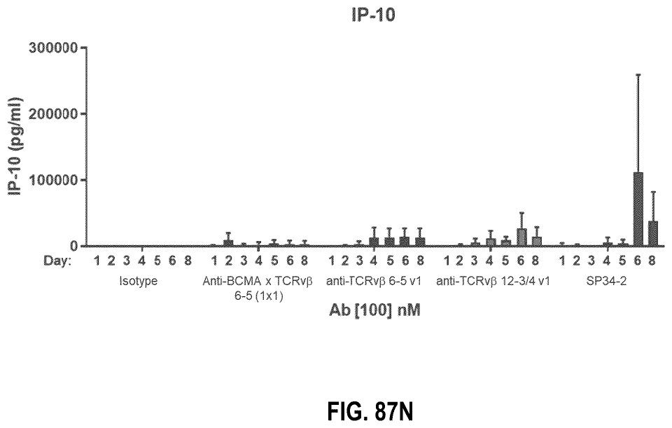

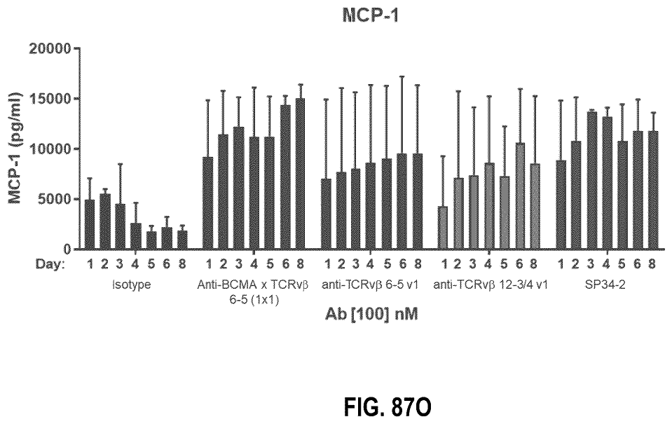

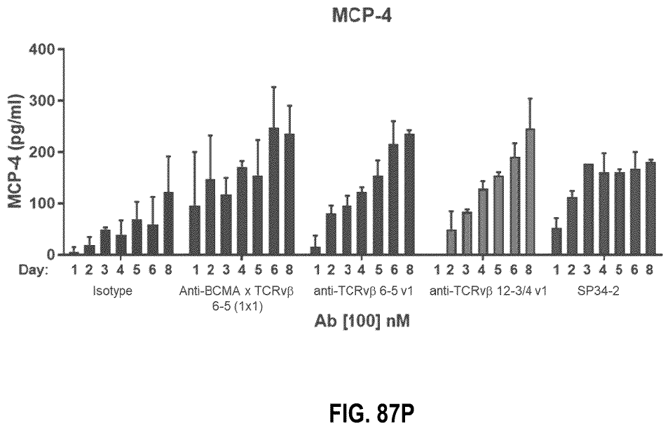

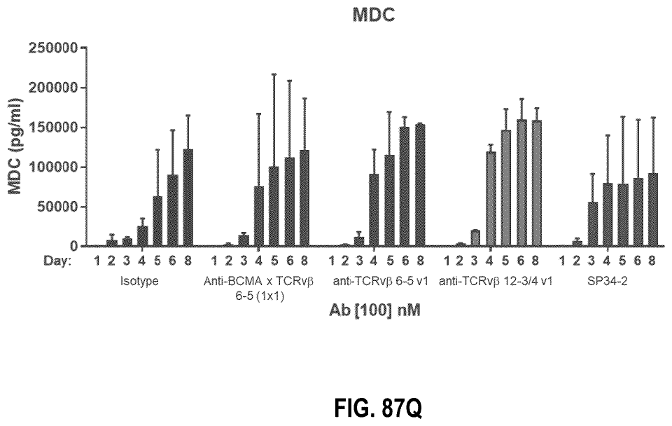

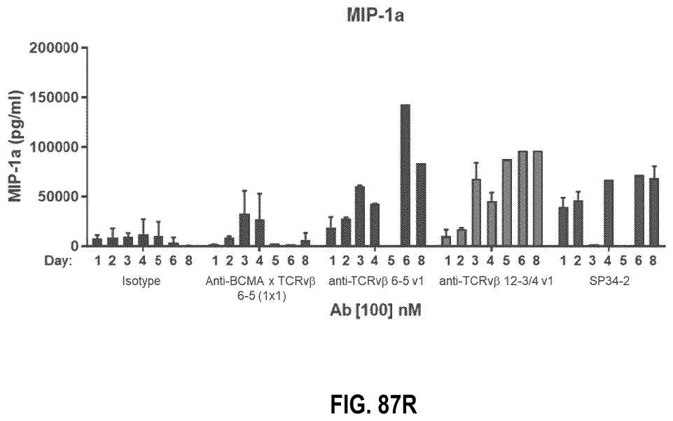

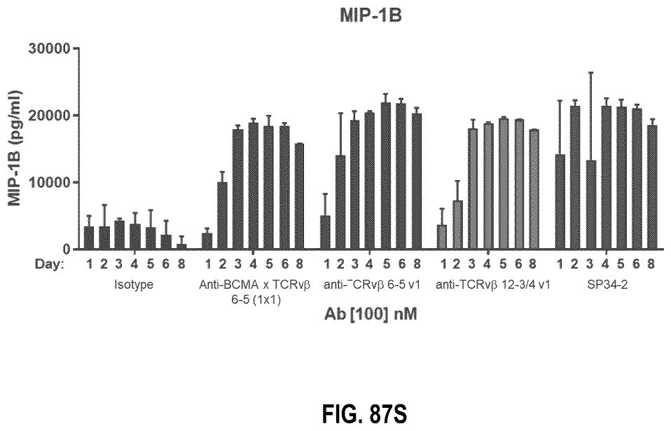

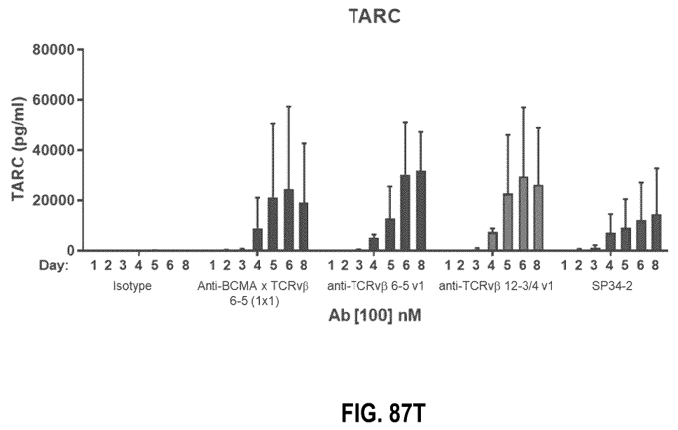

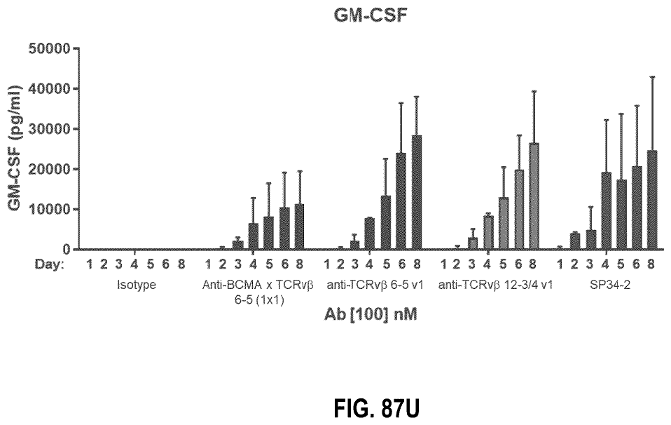

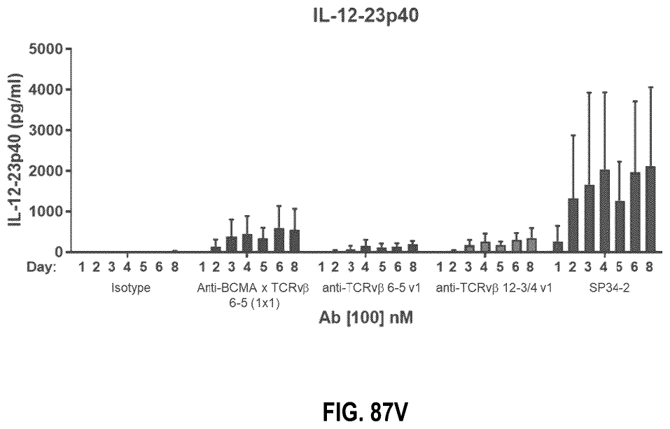

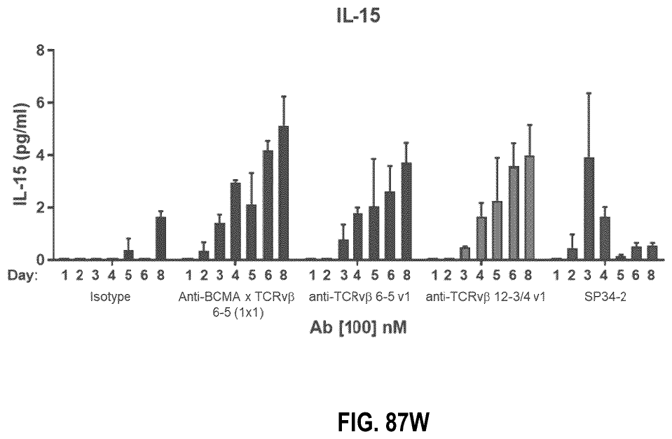

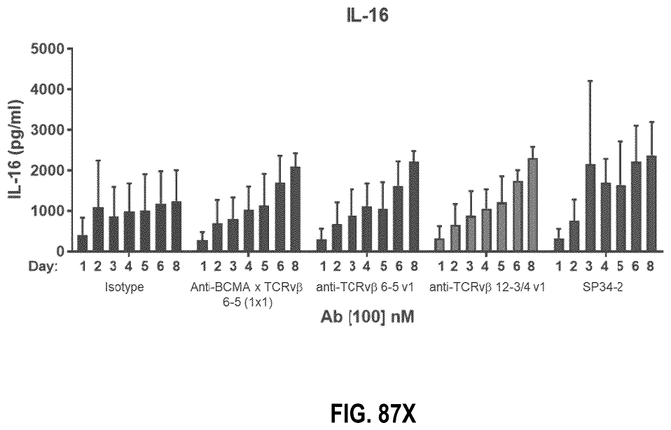

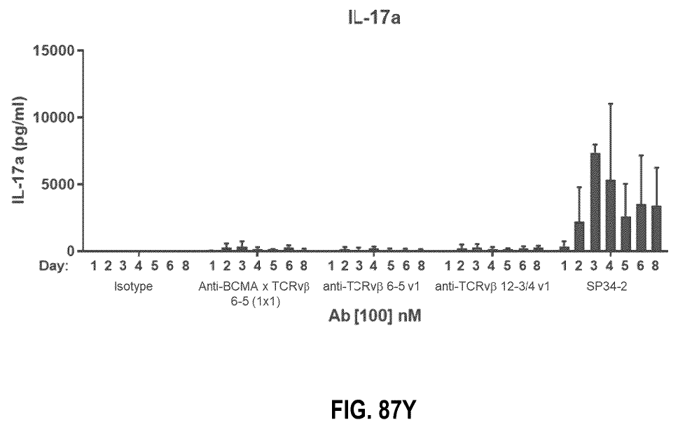

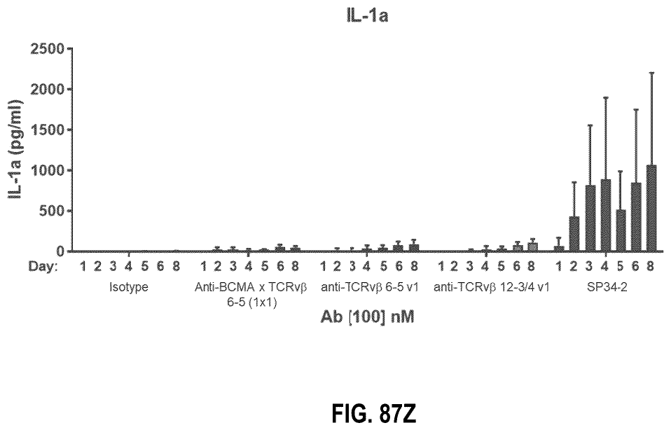

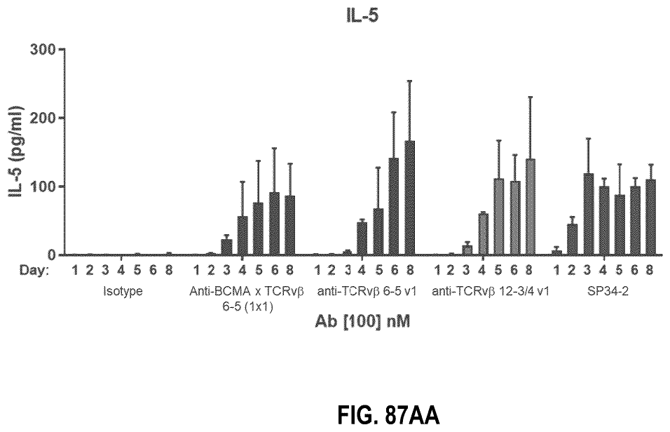

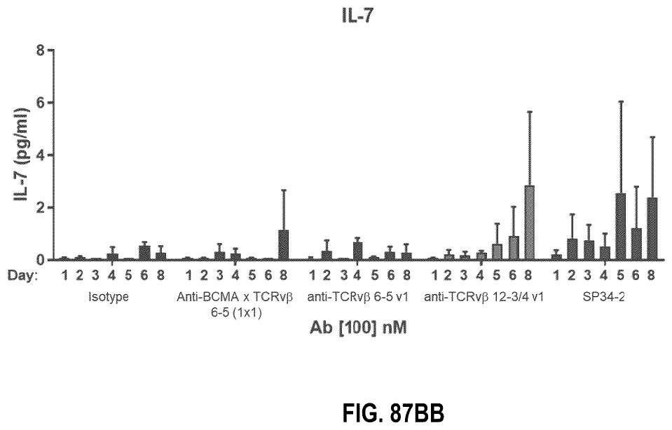

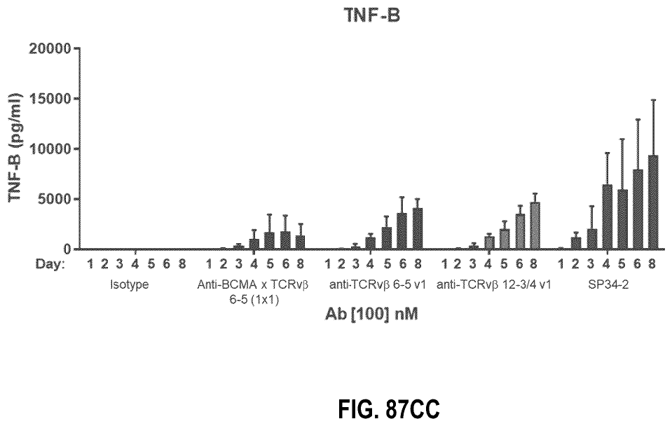

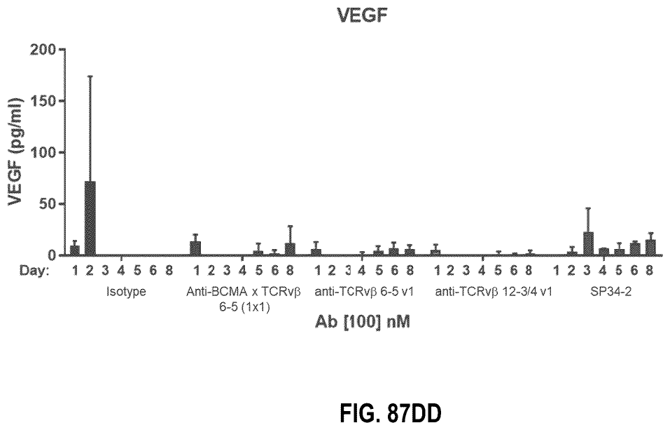

The data confirmed that T cells activated/expanded by either anti-TCRβV antibody, anti-TCRβV 6-5 v1 or anti-TCRvb 12-3/4 v1, as compared to either anti-CD3ε antibody (OKT3 or SP37-2) secreted a lower level of IFNγ (FIG. 87A), IL-1β (FIG. 87B), IL-4 (FIG. 87C), IL-6 (FIG. 87D), IL10 (FIG. 87E). TNFα (FIG. 87F); and higher levels of IL-2 (FIG. 87G). Secretion of IL-12p70 (FIG. 87H), IL-13 (FIG. 87I), IL-8 (FIG. 87J), Exotaxin (FIG. 87K), Exotaxin-3 (FIG. 87L), IL-8 (FIG. 87M), IP-10 (FIG. 87N), MCP-1 (FIG. 87O). MCP-4 (FIG. 87P), MDC (FIG. 87Q), MIP-1a (FIG. 87R), MIP-1b (FIG. 87S), TARC (FIG. 87T), GMCSF (FIG. 87U), IL-12-23p40 (FIG. 87V), IL-15 (FIG. 87W), IL-1β (FIG. 87X), IL-17a (FIG. 87Y), IL-1a (FIG. 87Z), IL-5 (FIG. 87AA), IL-7 (FIG. 87BB), TNF-β (FIG. 87CC), and VEGF (FIG. 87DD), were also tested.

In addition to determining the cytokine profile of T cells activated with the &TCRβV antibodies αTCRβV 6-5 v1 and αTCRβV 6-5 v2 (described above); the assays were conducted with additional TCRβV antibodies recognizing different clonotypes.

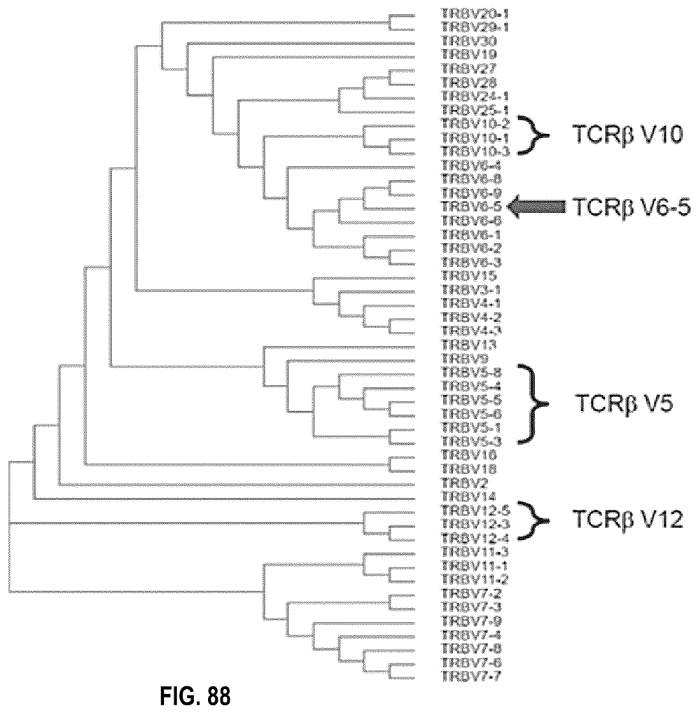

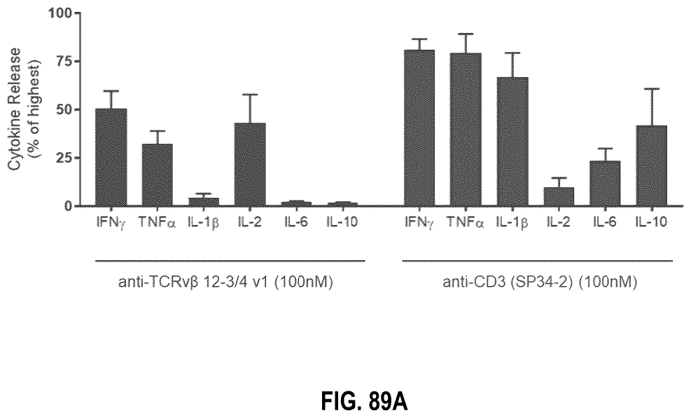

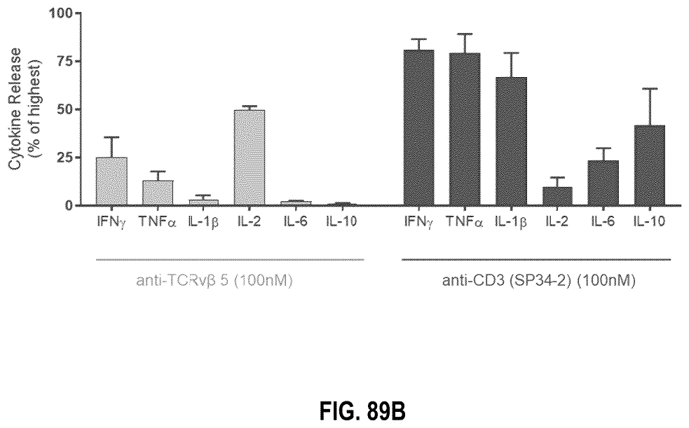

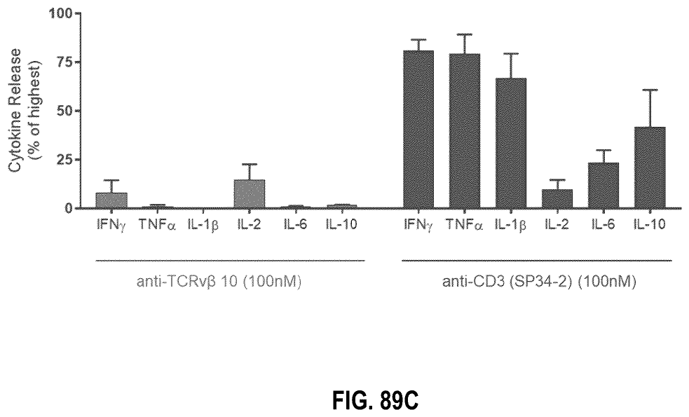

In one series of experiments antibodies tested included anti-TCRvb 12-3/4 v1, anti-TCRvb 10, and anti-TCRvb 5. Per the protocol described above, human PBMCs were solid-phase stimulated (plate-coated) with the indicated T cell-activating antibody (anti-TCRvb 12-3/4 v1, anti-TCRvb 10, anti-TCRvb 5, or the anti-CD3ε antibody SP34) at 100 nM. Supernatant were collected on day 1 to day 8; and cytokines were quantified using Meso Scale Discovery (MSD) assay FIG. 88 provides a graphical representation of sequences between the different clonotypes, highlighting the four subfamilies tested in this series of experiments. PBMCs activated/expanded with the anti-TCRvb 12-3/4 v1 antibody (FIG. 89A), anti-TCRvb 10 antibody (FIG. 89B), or anti-TCRvb antibody (FIG. 89C) exhibited lower levels of secretion of cytokines associated with cytokine release syndrome, including IFNγ, TNFα, IL-1β, IL-2, IL-6, and IL-10, as compared to PBMCs activated/expanded with the anti-CD3ε antibody SP34-2.

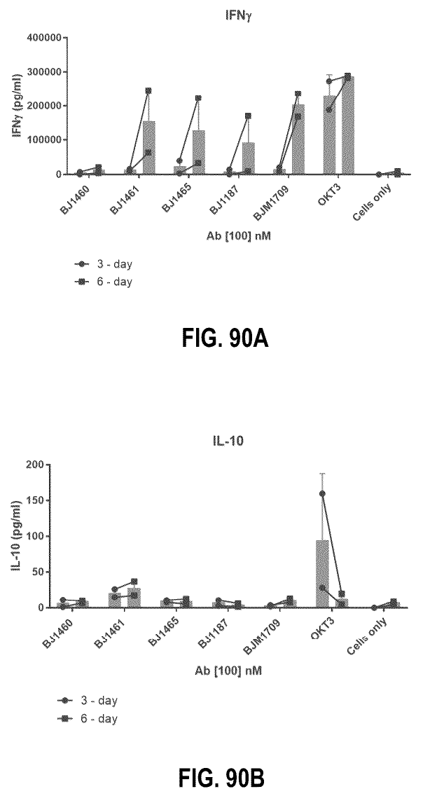

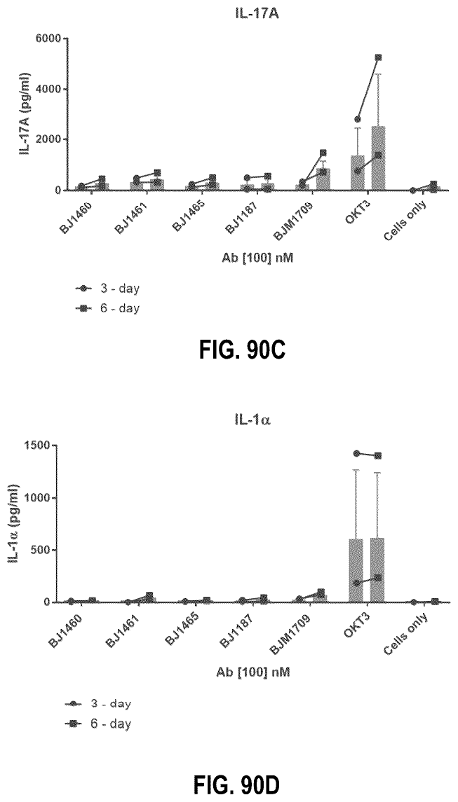

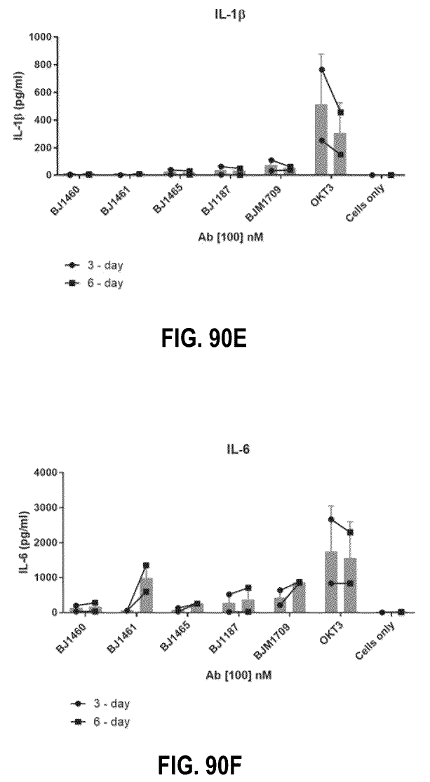

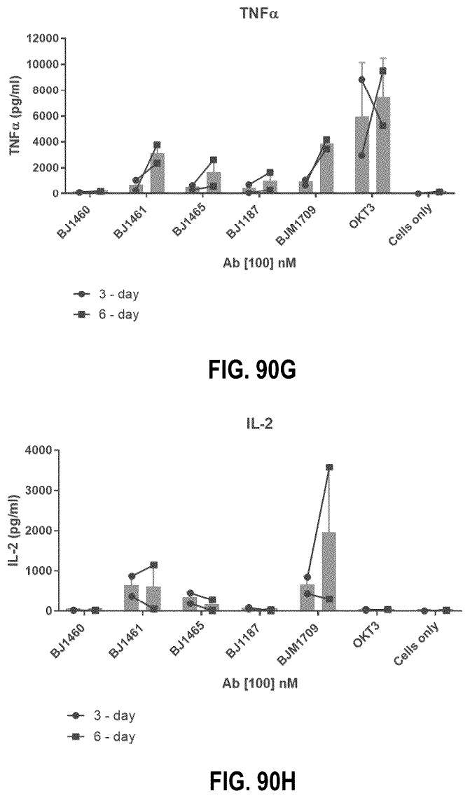

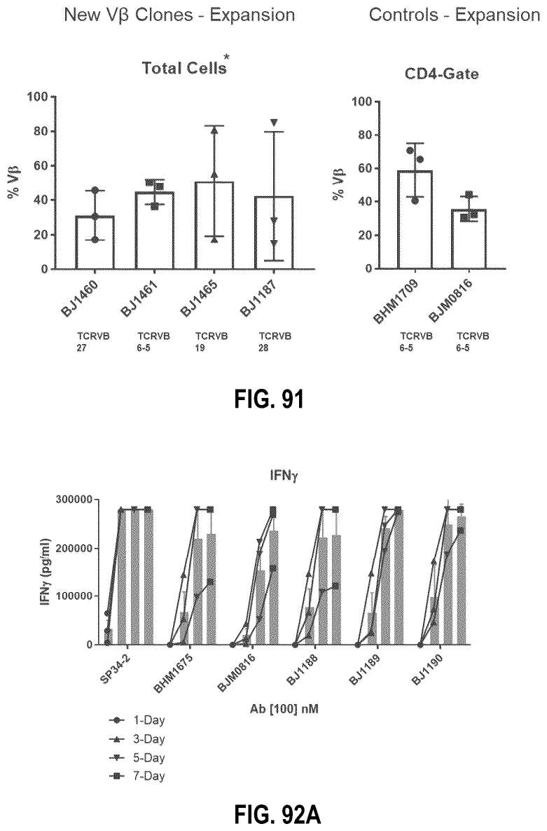

In a second series of experiments, antibodies tested included the anti-TCRVβ antibodies: BJ1460, BJ1461, BJ1465, BJ1187, BJM1709; the anti-CD3ε antibody OKT3, and a cell only control. At Day-0 PBMCs from donor 10749 were thawed and counted along with PBMCs from two fresh donors (13836 and 14828). 200,000 PBMCs in 180 μL of X-vivo media/well (1×10e6 cells/mL) was added to a round bottom 96 well plate-one donor for 1/3 of the plate. 20 μL of 10×TCRVβ antibodies at 100 nM or 15 μg/mL were added to the wells of the plate and one triplicate of wells was added with cells only. The pate was kept in a 37° C. incubator with 5% CO₂. The cells were stimulated for 3 days with a selected antibody and 50 μL of supernatant harvested from the plate and stored at −20° C. 50 μL of media was added back to each well and the plate kept in a 37° C., incubator with 5% CO₂. On Day-6 50 uL of supernatant was harvested from each well of the plate and stored at −20° C. The cells from two wells out of the triplicate were combined and media replenished with huIL-2 was added the cell suspension for each donor was transferred into a 12-well plate. The cells were incubated overnight to allow for rest and expansion in IL-2. The cells were subsequently stained for specific Vβ-clones for detection of specific Vβ-clone expansion by FACS analysis. The concentration of cytokines (including IFNγ, IL-10, IL-17A, IL-1α, IL-1β, IL-2, IL-6, and TNFα) in the media were analyzed in the Day-3 and Day-6 supernatant samples using Meso Scale Discovery (MSD) assay. The data confirmed that PBMCs cells activated/expanded using any of the anti-TCRβV antibodies—BJ1460, BJ1461, BJ1465, BJ1187, BJM1709—secreted lower levels of IFNγ (FIG. 90A), IL-10 (FIG. 90B), IL-17A (FIG. 90C), IL-1α (FIG. 90D), IL-1β (FIG. 90E), IL-6 (FIG. 90F), TNFα (FIG. 90G); and higher levels of IL-2 (FIG. 90H). FACS analysis further showed expansion of T cells expressing the indicated TCRVβ clones (FIG. 91).

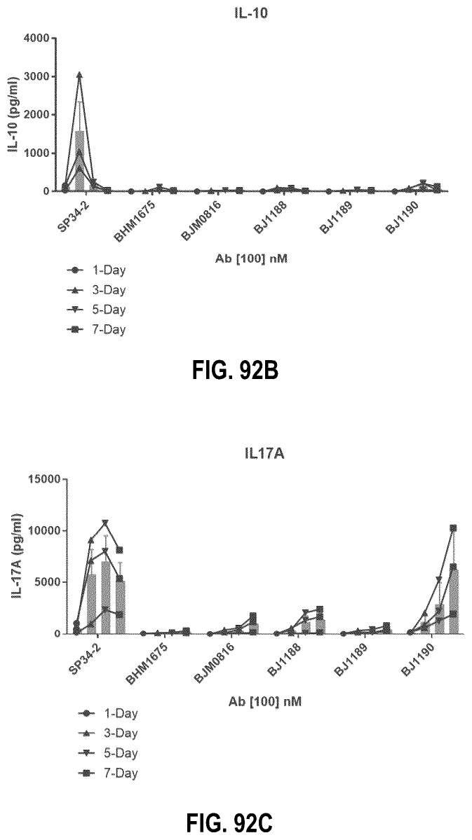

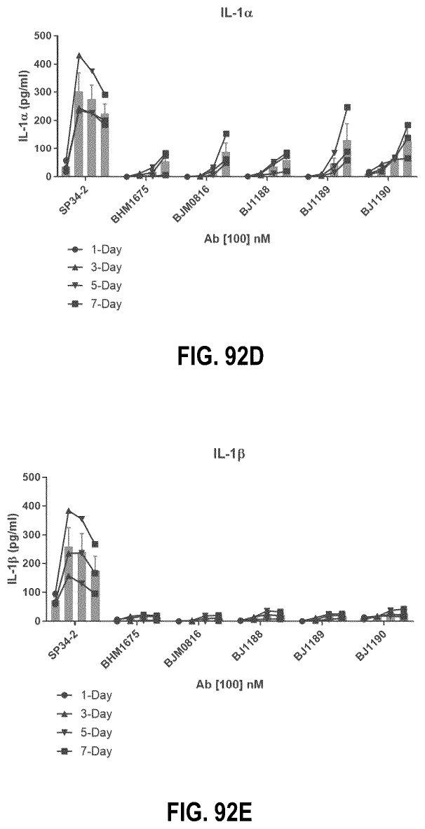

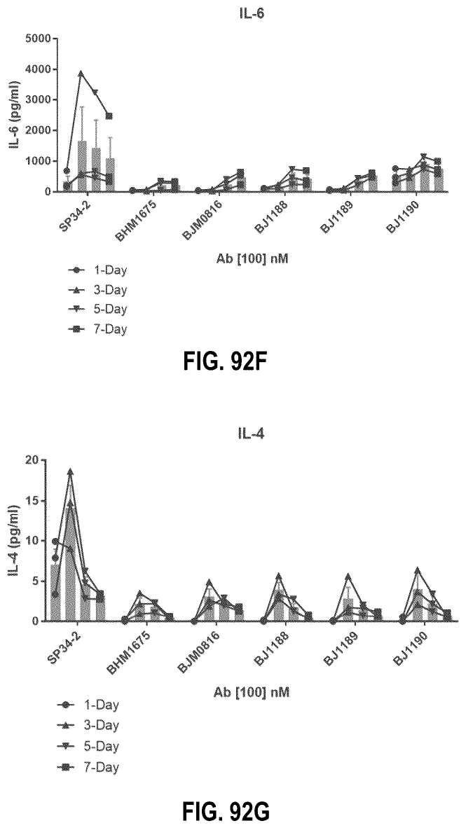

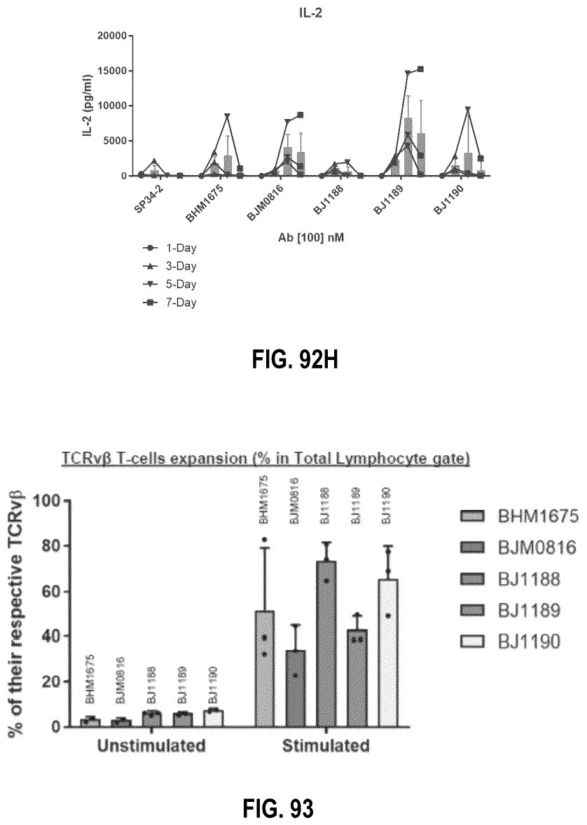

In a third series of experiments, antibodies tested included the anti-TCRVβ antibodies: BHM1675, BJM0816, BJ1188, BJ1189, BJ1190; and the anti-CD3ε antibody SP34-2. The indicated antibodies were coated into a 96-well round bottom plate at concentration of 100 nM or 15 μg/mL at 200 μl/well in PBS at 4° C. overnight or at 37° C., for a minimum of 2 hours. The plate was washed the next day with 200 μL of PBS and 0.2 ×10\hat{ }6 PBMCs/well from donors: CTL_123, CTL_323 and CTL_392. Supernatant samples were collected on days 1, 3, 5, and 7. A 10-plex Meso Scale Discovery (MSD) assay was run on the supernatants to determine the concentration of cytokines (including IFNγ, IL-10, IL-17A, IL-1α, IL-1β, IL-6, IL-4, and IL-2). After day 7, cells were pelleted and added to culture medium supplemented with IL-2 for one additional day to allow for expansion. Expansion of T cells expressing TCRVβ clones was analyzed by FACS staining using the same activating antibody followed by a secondary anti-human/mouse FITC antibody. Live/Dead, CD4+ and CD8+ T cells were also stained for using BHM1675, BJM0816, BJ1189 and BJ1190 antibodies. The data confirmed that PBMCs cells activated/expanded using any of the anti-TCRβV antibodies—BHM1675, BJM0816, BJ1188, BJ1189, BJ1190—secreted lower levels of IFNγ (FIG. 92A), IL-10 (FIG. 92B), IL-17A (FIG. 92C), IL-1α (FIG. 92D), IL-1β (FIG. 92E), IL-6 (FIG. 92F), IL-4 (FIG. 92G); and higher levels of IL-2 (FIG. 92H). FACS analysis further showed that TCRVβ sub-clone T-cells are expanded by their respective activation antibody (FIG. 93).

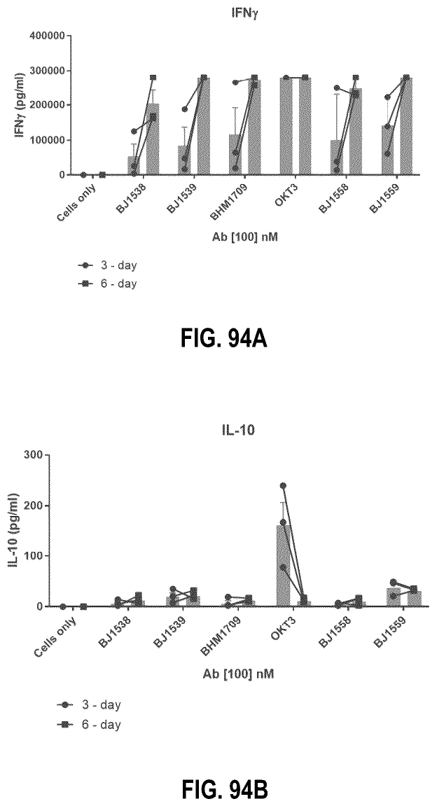

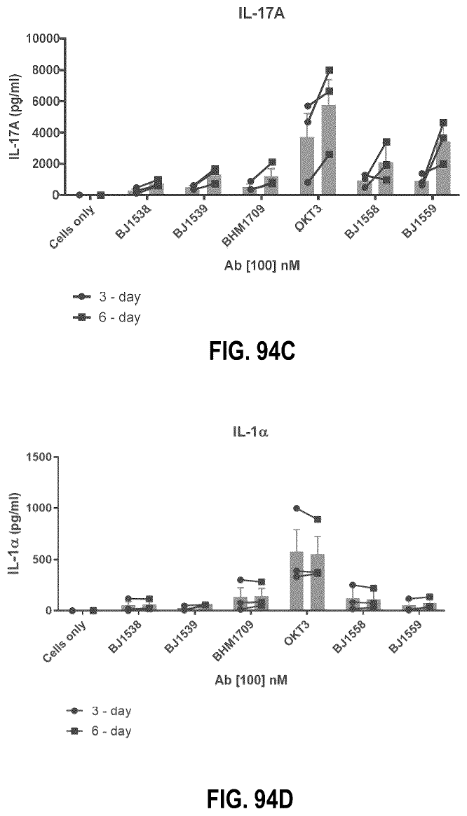

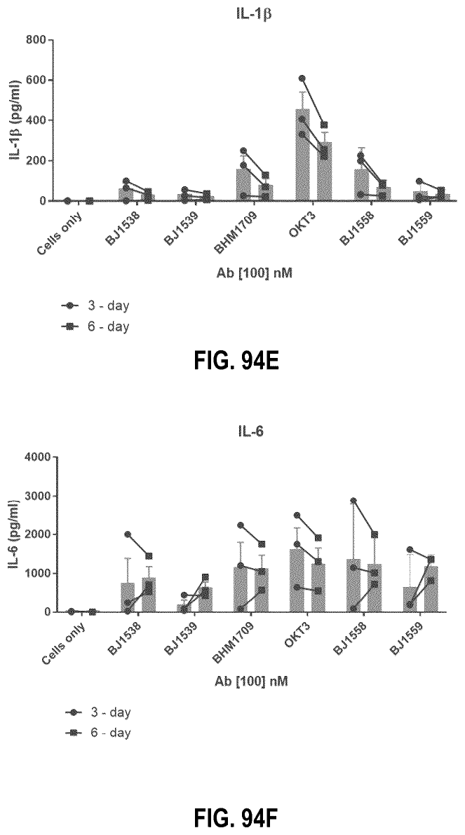

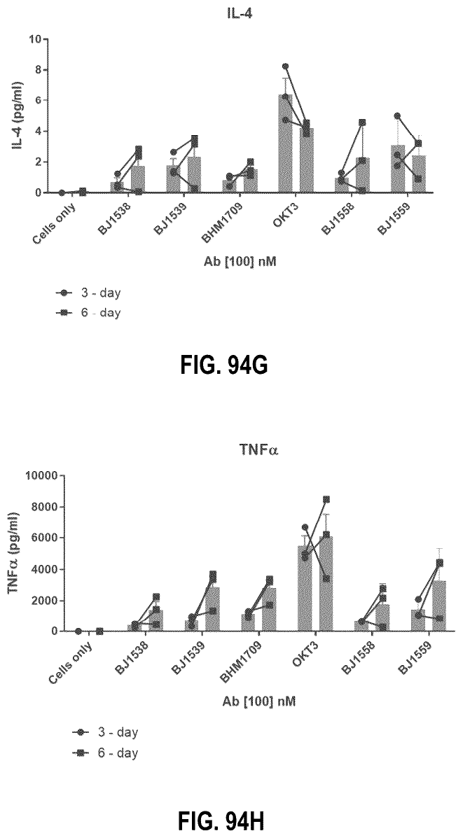

In a fourth series of experiments, antibodies tested included the anti-TCRVβ antibodies: BJ1538. BJ1539, BJ1558, BJ1559, BHM1709; and the anti-CD3ε antibody OKT3. The indicated antibodies were coated into a 96-well round bottom plate at concentration of 100 nM or 15 μg/mL at 200 μl/well in PBS at 4° C. overnight or at 37° C., for a minimum of 2 hours. The plate was washed the next day with 200 μL of PBS and 0.2 ×10\hat{ }6 PBMCs/well from donors: 10749, 5078 and 15562 (frozen and thawed samples). Supernatant samples were collected on days 3 and 6. A 10-plex Meso Scale Discovery (MSD) assay was run on the superatants to determine the concentration of cytokines (including IFNγ, IL-10, IL-17A, IL-1α, IL-1β, IL-6, IL-4, TNFα, and IL-2). The data confirmed that PBMCs cells activated/expanded using any of the anti-TCRβV antibodies—BJ1538, BJ1539, BJ1558, BJ1559, BHM1709—secreted lower levels of IFNγ (FIG. 94A), IL-10 (FIG. 94B), IL-17A (FIG. 94C), IL-1a (FIG. 94D), IL-1β (FIG. 94E), IL-6 (FIG. 94F), IL-4 (FIG. 94G) TNFα (FIG. 94H); and higher levels of IL-2 (FIG. 94I).

In summary, the data shows that anti-TCRvb antibodies recognizing different TCRvb subfamilies (or subtypes) have a similar cytokine profile and do not induce cytokines associated with CRS.

### Example 29: Anti-TCRvb does not Activate T Cells without Cross-Linking

To assess whether bivalent anti-TCRvb antibodies activate T cells without cross-linking-purified T cells from 2 donors were stimulated with anti-TCRvb (TCRvb 6-5 v1) or anti-CD3e (SP34), either plate-coated or in solution. Supernatants were collected at day 1, 3, 5 and 7 post activation. Cytokine secretion was detected using MSD 10 plex kit (IFN-g, IL-10, IL-15, IL-17A, IL-1a, IL-1b, IL-2, IL-4, IL-6 and TNF-a).

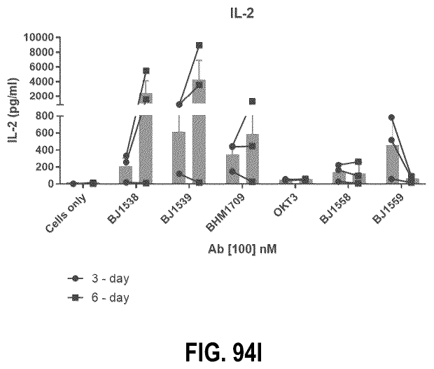

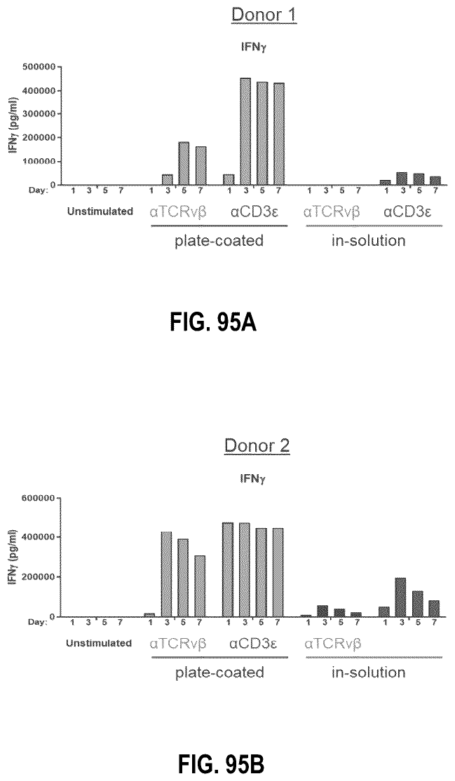

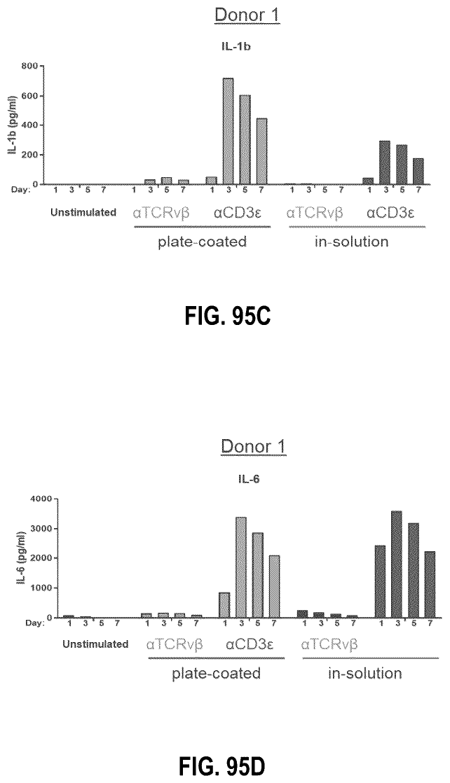

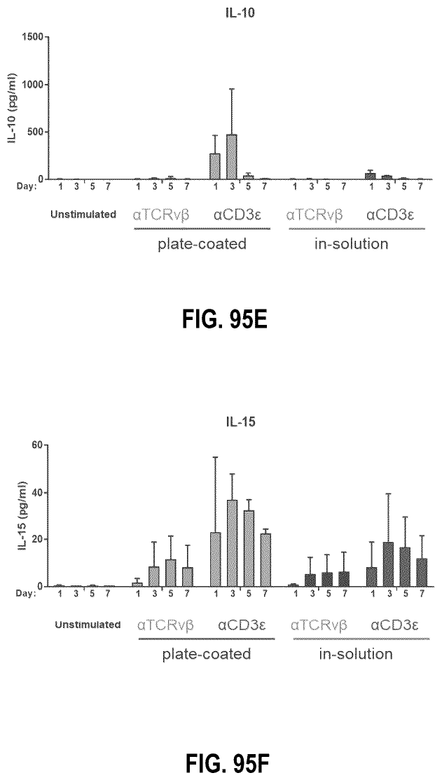

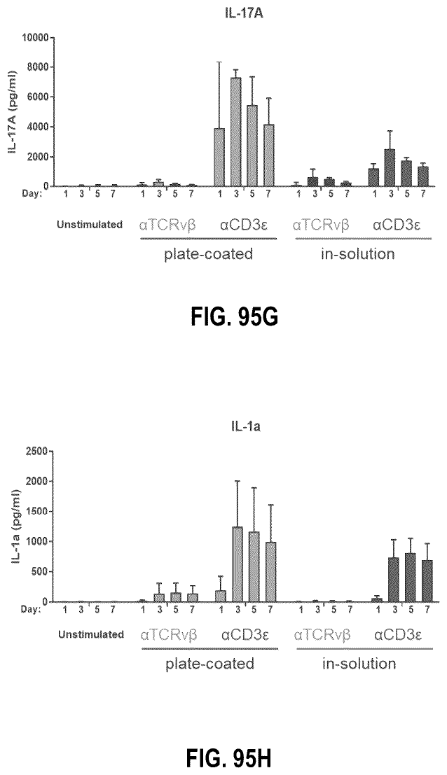

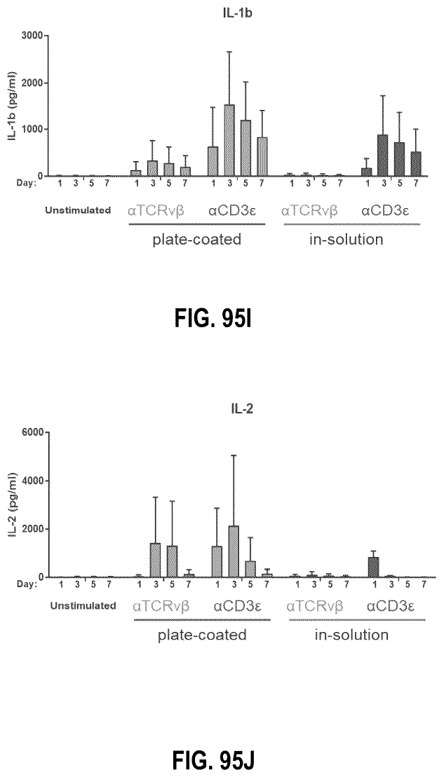

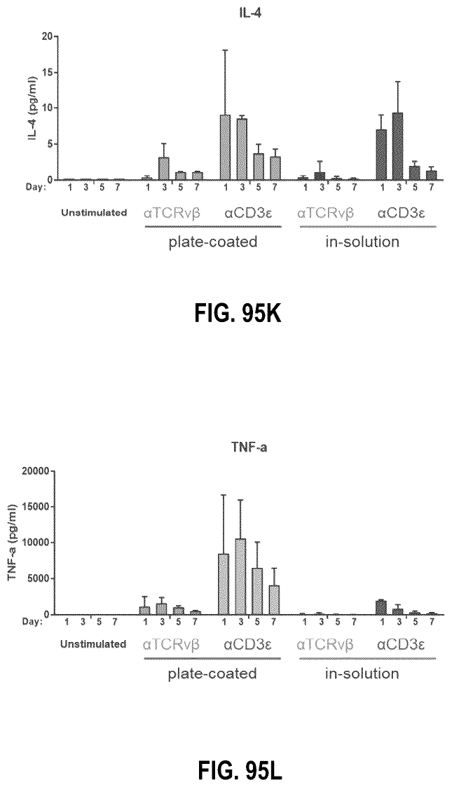

The results show the PBMCs activated/expanded with anti-TCRvb 6-5 v1 antibody in solution do no induce very little IFNγ secretion as compared to PBMCs activated/expanded with anti-TCRvb 6-5 v1 antibody in immobilized (allowing for crosslinking) (FIG. 95A and FIG. 95B). The results show the PBMCs activated/expanded with anti-TCRvb 6-5 v1 antibody in solution do no induce very little or no IL-1b (FIG. 95C), IL-10 (FIG. 95E), IL-15 (FIG. 95F), IL-17A (FIG. 95G), IL-Ja (FIG. 95H), IL-1b (FIG. 95I), IL-2 (FIG. 95J), IL-4 (FIG. 95K), IL-6 (FIG. 95D), and TNF-α (FIG. 95L) secretion. In summary, the data shows that anti-CD3ε activates T cells in solution (without crosslinking); while the anti-TCRvb antibodies does not activate T-cells in solution.

### Example 30: Competition of Binding to TCRVB by Two Anti-TCRVβ 5-5,5-6 Antibodies with Distinct Sequences

This Example describes epitope competition of two anti-TCRVβ 5-5.5-6 antibodies for binding to their shared TCRVB antigen. The TM23 and MH3-2 antibodies both bind to TCRVβ 5-5.5-6. However, the TM23 and MH3-2 antibodies do not share substantial sequence homology.

As shown in FIG. 25A-25B, anti-TCRβV antibody molecules as described herein recognize a structurally conserved domain on the TCRBV protein (as denoted by the circled area in FIG. 25A), but have low sequence similarity among themselves. To test whether two anti-TCRVβ 5-5,5-6 antibodies which do not share substantial sequence homology, can compete for binding to the TCRBV antigen, a competition assay was carried out.

Purified MH3-2 antibody was conjugated to AF647. T cells from two donors were preincubated with 500 nM of the TM23 antibody or left untreated. The T cells were then stained with the MH3-2 antibody conjugated to AF647.

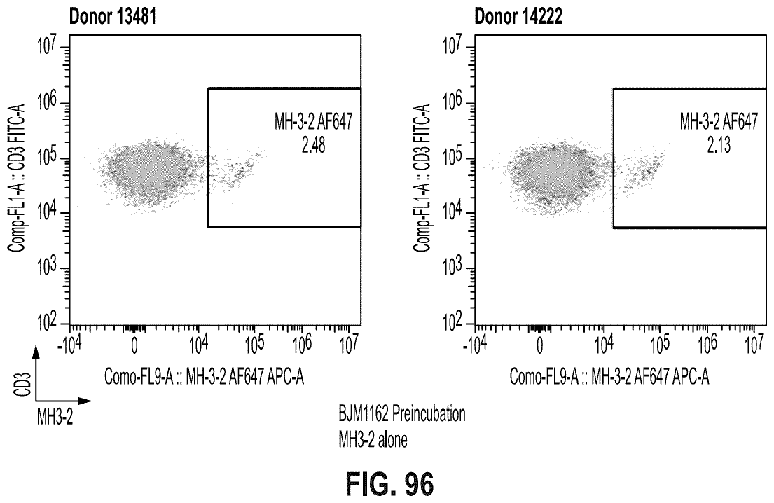

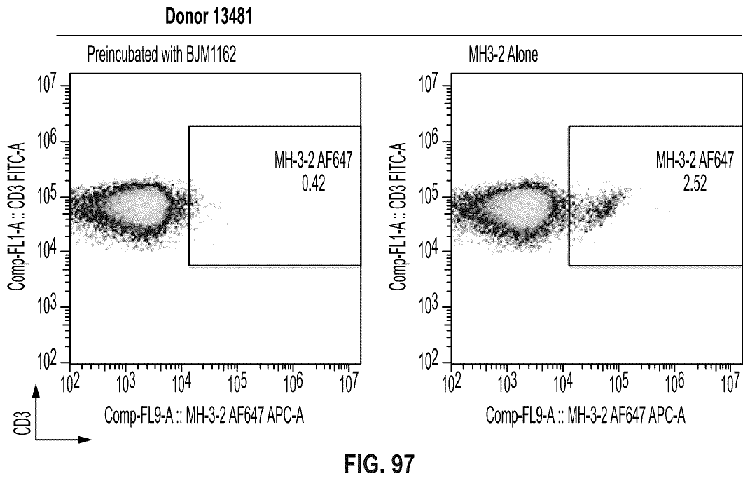

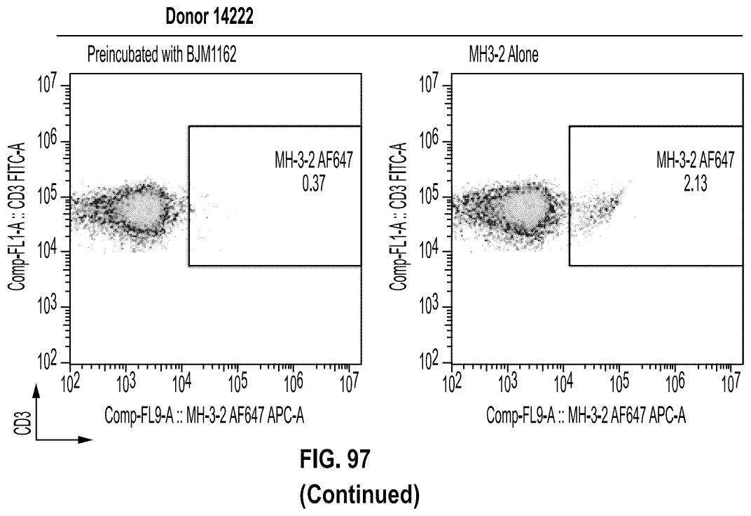

The results show that preincubation of T cells with the TM23 antibody blocks binding of MH3-2 (FIG. 96 and FIG. 97). The data shows that the TM23 antibody competes for binding with the same epitope as the MH3-2 antibody, despite both antibodies having diverse sequences. This data confirms the observation that anti-TCRβV antibody molecules which have low sequence similarities among themselves, bind and recognize a structurally conserved epitope on the TCRβV protein.

### Example 31: Polyfunctional Strength Index of Anti-TCRVβ 6-5 Antibody Expanded T Cells

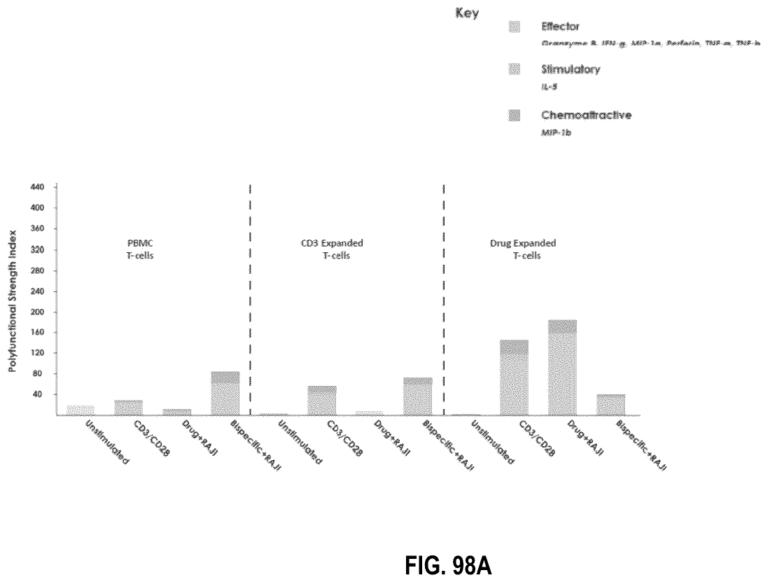

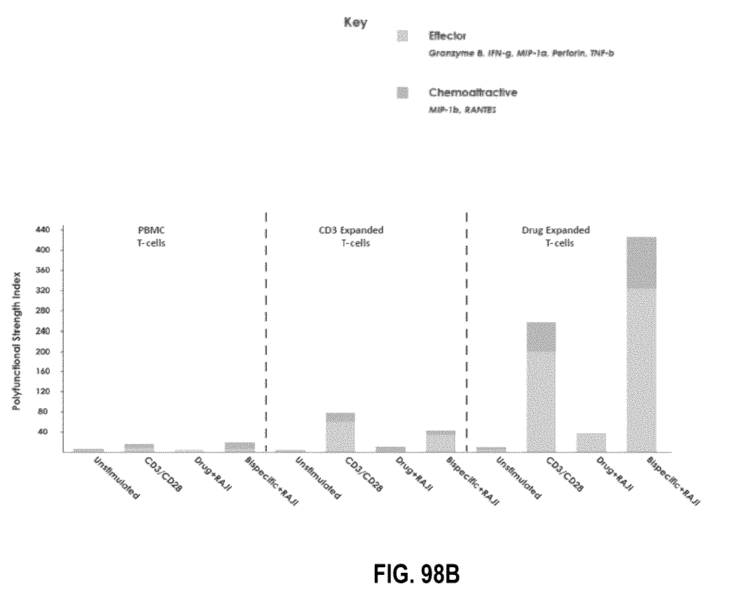

The polyfunctional strength index (PSI) of PBMCs were compared to anti-CD3ε antibody expanded CD4+ T cell (FIG. 98A) and CD8+ T cells (FIG. 98B) and anti-TCRVβ 6-5 antibody expanded (Drug Expanded T cells) CD4+ T cells (FIG. 98A) and CD8+ T cells (FIG. 98B). PSI is defined as the percentage of polyfunctional cells in the sample, multiplied by the intensities of the secreted cytokines. The data shows that there is a greater upregulation of PSI in the CD4+ T cells (FIG. 98A) and CD8+ T cells (FIG. 98B) across the groups expanded with anti-TCRVβ 6-5 antibody.

### Example 32: Binding of the Multifunctional Polypeptide Molecule as Described Herein to Soluble TCR And Jurkat Cells Expressing TCR

In this Example, the binding affinity of a multifunctional polypeptide molecule as described herein for the TCR is tested.

Jurkat cells expressing TCR are stained with increasing concentrations of a multifunctional polypeptide molecule as described herein or a control TCRvb antibody at 4 Celsius for 30 min. Subsequently, the cells are washed with PBS buffer and antibodies bound to the surface of the cells are detected by PE-labeled anti-human Fc antibody. The percentage of the positive stained cells are blotted against the concentration. The binding of the multifunctional polypeptide molecule as described herein to soluble TCR is plotted as a graph. The Kd of the multifunctional polypeptide molecule as described herein to bind soluble TCR is calculated.

The multifunctional polypeptide molecule as described herein is immobilized on a CM5 Series S Sensor Chip via Anti-human Fc antibody to 50 RU. Soluble TRBV antigen is diluted to, e.g., 500 nM and then serially diluted two-fold. The time duration for association and dissociation is measured. This assay is run in 1×HBS-EP+ Buffer pH 7.4 and at 25C. It is evaluated whether the data is fit using a 1:1 binding model. The binding of the multifunctional polypeptide molecule as described herein or the control anti-TCRvb antibody to TCR expressed on Jurkat cells is plotted as a graph. The EC50 for the multifunctional polypeptide molecule as described herein is calculated and compared with the EC50 for the anti-TCRvb antibody.

### Example 33: In Vitro and In Vivo Characterization of a Multifunctional Polypeptide Molecule as Described Herein

This Examples describes the characterization of a murine anti-TCRvb antibody and the multifunctional polypeptide molecule as described herein. Similar to the human clonotypes (subfamilies), the TCRb variable chain locus in mice consists of 31 different families with a total of 35 subfamilies of which 23 are functionally expressed. A surrogate TCRvb clonotypical antibody for mice strain C57BL/6 has been identified which shares similar characteristics with the human TCRvb antibodies. This anti-mouse TCRvb antibody binds specifically to TCRvb in C57BL/6 mice which are expressed on approximately 15% of all T cells. Similar to the human TCRvb specific antibodies, this murine TCRvb specific antibody induces murine T cell proliferation and a similar cytokine profile in vitro. The discovery of anti-TCRvb antibody enables the evaluation of TCRvb-mediated T cell activation and re-directed cell killing in fully immuno-competent mice models, as well as to assess memory anti-tumor response in vivo.

First, the in vitro functional activity of the multifunctional polypeptide molecule as described herein is tested Splenic mononuclear cells are freshly isolated from C57BL6 mice, treated with the multifunctional polypeptide molecule as described herein. The isolated cells are assessed for TCRvβ+ T cell binding, expansion and activation. Cells are treated with, e.g., 0.0008-200 nM doses (4-fold dilutions) of the multifunctional polypeptide molecule as described herein in RPMI-1640 with 10% FBS, or medium alone, for, e.g., 6 days. On, e.g., day 3 and 6, cells are analyzed by flow cytometry using the exemplary antibodies shown below:

TABLE 18

| Primary Ab | Secondary Ab |
|---|---|
| eFluor780 | Viability |
| CD20 | A647 |
| CD3 | BV421 |
| TCRvβ 13-2/3 | PE |
| CD25 | FITC |

The multifunctional polypeptide molecule as described herein bound specifically to splenic T cells from C57BL6 mice is plotted as a graph. Activation and expansion of m TCRvβ+ T cells is analyzed at, e.g., day 3 and 6, respectively. The in vitro characterization of the multifunctional polypeptide molecule as described herein is expected to show that the multifunctional polypeptide molecule as described herein serves as a surrogate tool for syngeneic tumor model studies.

Next, in vivo experiments are performed with the multifunctional polypeptide molecule as described herein. On day 0, 8 week old female C57BL/6 mice are randomized in to three arms (e.g., n=5/arm) based on body weight. Mice are intravenously injected once either with PBS, e.g., 0.1 mg/kg and 1 mg/kg of the multifunctional polypeptide molecule as described herein. On Day 3 mice are sacrificed and harvested for whole blood and spleen. Tissues are subjected to Flow cytometry and checked for B-cells. NK cells. TCRvb+ cells and CD3+ cells.

The multifunctional polypeptide molecule as described herein is expected to expand mouse NK cells in vivo, in the blood and spleen. The study is also expected to demonstrate whether the multifunctional polypeptide molecule as described herein is tolerated at the indicated doses and duration of study.

### Example 34: Target Cell Lysis and Cytokine Profile of the Multifunctional Polypeptide Molecule as Described Herein

This Example describes potent lysis of target cells and reduced CRS associated cytokine secretion with the multifunctional polypeptide molecule as described herein.

To test target cell killing, αTCRvβ pre-expanded T cells are incubated with target cells, e.g., Raji target cells in the presence of the multifunctional polypeptide molecule as described herein or αTCRvβ antibody for 24 hours. Target cell lysis is assessed by a KILR Cytotoxicity and Cytokine Quantification as follows. Human PBMCs are isolated from whole blood. From isolated PBMC's, human CD3+ T cells are isolated using magnetic-bead separation (negative selection) (Miltenyi biotec) and activated by immobilized (plate-coated) anti-TCR Vβ13.1 (e.g., A-H.1 or BHM1709) at, e.g., 100 nM for, e.g., 6 days. Activated T-cells (from plate-coated) are then transferred and expanded in tissue culture flask at a concentration of, e.g., 50 U/ml for an additional 2 days. Expanded TCR Vβ13.1 are washed and co-cultured in the presence of target cells (e.g., Raji Cells) at a E:T ratio of 5:1 and serial diluted concentration of the multifunctional polypeptide molecule as described herein and anti-TCR Vβ (serving as control) for, e.g., 24 hours. Post, e.g., 24 hours, cell co-culture supernatants are collected and quantified for specific target cell death. Target cells (Raji cells) are a KILR-retroparticles reporter cell assay (DiscoverX).

KILR-Raji Target cells are engineered to stably express a protein tagged with enhanced ProLabel (ePL), a β-gal reporter fragment, using the KILR Retroparticles, and when its membrane is compromised due to cell death, it will release the tagged protein into the media. The KILR reporter protein is detected in the media/supernatant by the addition of detection reagents containing the enzyme acceptor (EA) fragment of the β-gal reporter. This leads to the formation of the active β-gal enzyme which hydrolyzes the substrate to give a chemiluminescent output (RLU). Percentage (%) of target cell death is calculated using the following formula: (RLU_{Treatment}−RLU_{No Treatment})/(RLU_{Maximum Lysis}−RLU_{No Treatment})×100. The pre-expanded T cells (TrEKs) by the multifunctional polypeptide molecule as described herein are expected to demonstrate efficient killing of Raji target cell at low effector. The multifunctional polypeptide molecule as described herein may require time for differentiation and expansion of TCRvβ+ T cells.

To determine if the lack of CRS associated cytokine induction by immobilized anti-TCRvβ antibodies can be recapitulated by the multifunctional polypeptide molecule as described herein, human PBMCs are incubated in the presence of T cell-activating multispecific molecules polypeptide molecule as described herein at, e.g., 3 nM. Supernatant are collected on day 1 to day 6, and cytokines are quantified by using MSD. The multifunctional polypeptide molecule as described herein may show increased cytokine production against a backdrop of delayed and reduced levels of CRS-related cytokines.

### Example 35: Cytokine Profile of the Multifunctional Polypeptide Molecule as Described Herein

This Example describes cytokines secreted by PBMCs following activation by the multifunctional polypeptide molecule as described herein. For comparison, activation by an anti-TCR beta constant 1 (TRBC1) antibody is also analyzed.

Briefly, human PBMCs are isolated from whole blood followed by solid-phase (plate-coated) stimulation with Molecule Hor Antibody F at 100 nM. Supernatant is collected on, e.g., Days 1, 2, 3, and 5 (for Molecule H) or Days 2 and 5 (for Antibody F) followed by multiplex cytokine analysis for, e.g., IFNγ, IL-2, IL-1β, IL-6, IL-10, and TNFα, quantified using MSD (Meso Scale Discovery) platform, following the manufacturer's protocol. The cytokine profile of the multifunctional polypeptide molecule as described herein is expected to be different from that of the anti-CD3 antibody OKT3 or the anti-TRBC1 Antibody F.

### Example 36: Pharmacokinetic (PK) Profile of the Multifunctional Polypeptide Molecule as Described Herein in Mice

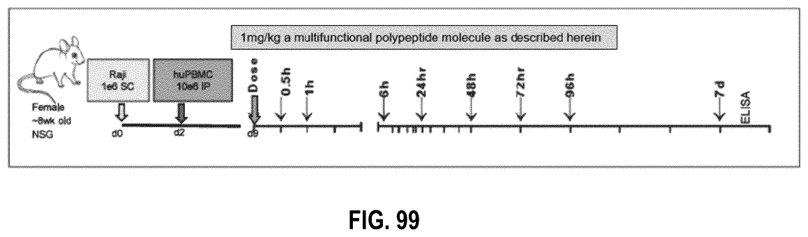

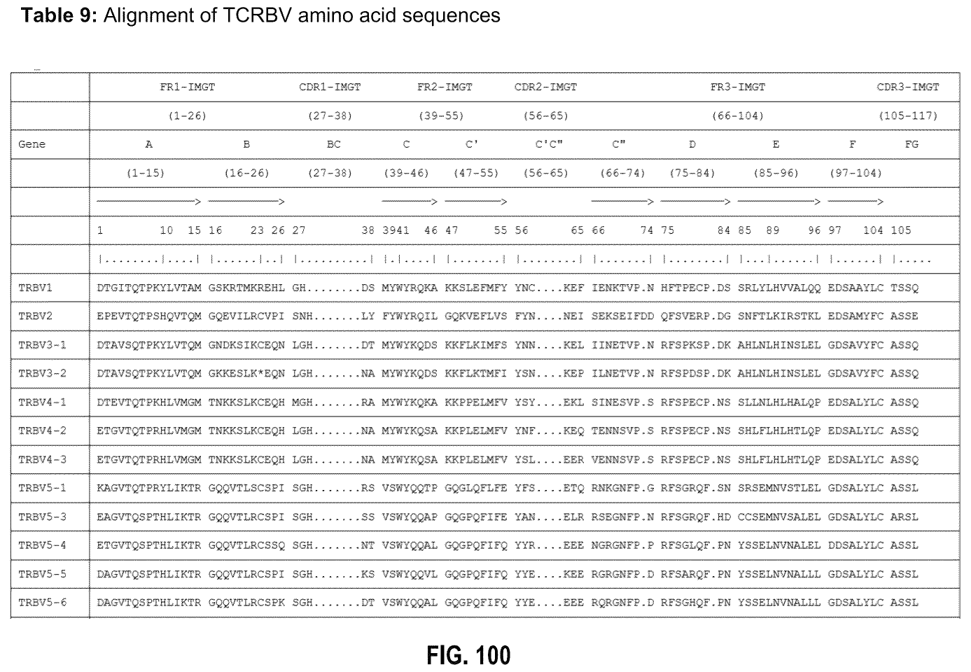

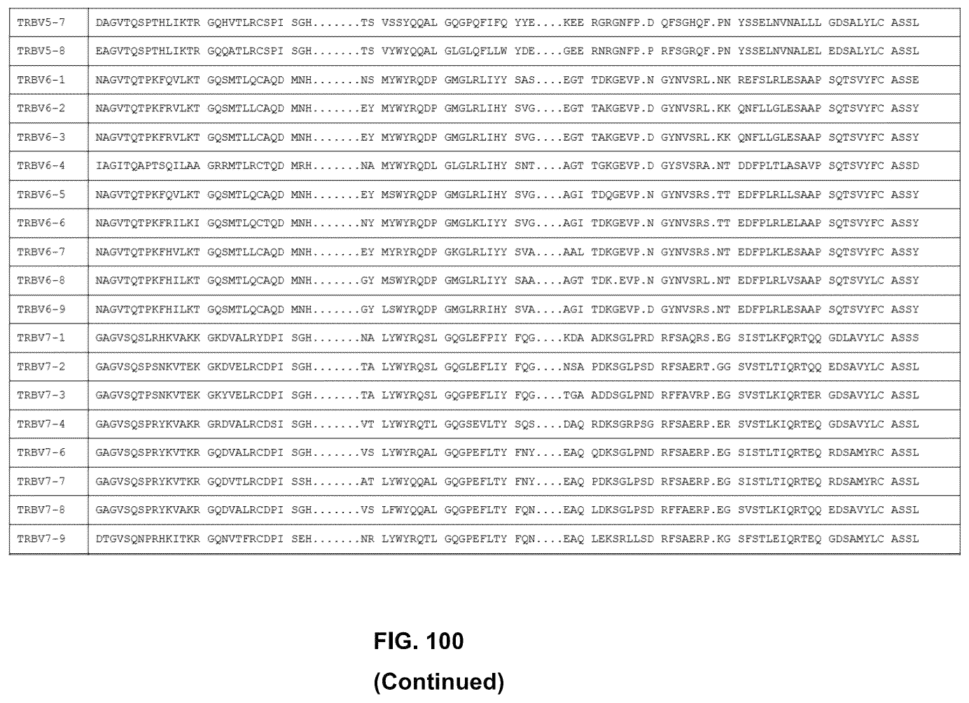

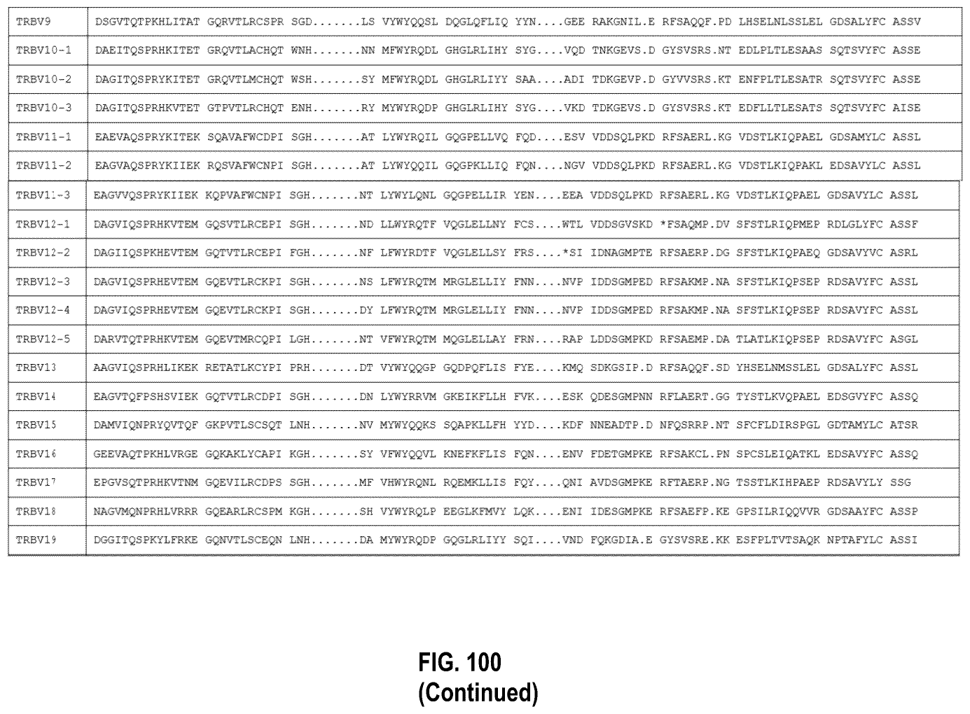

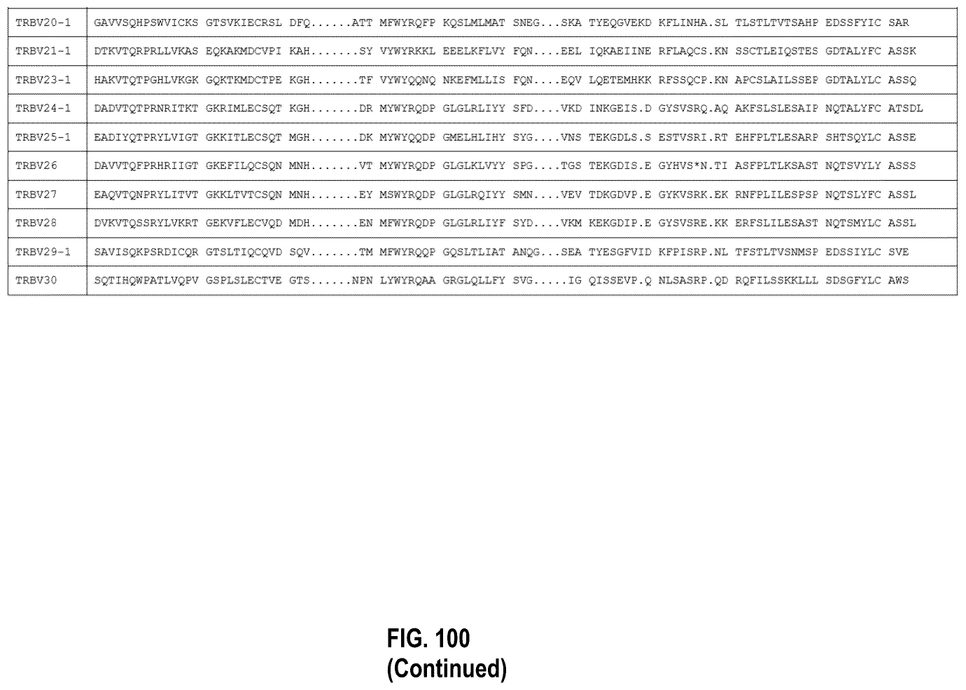

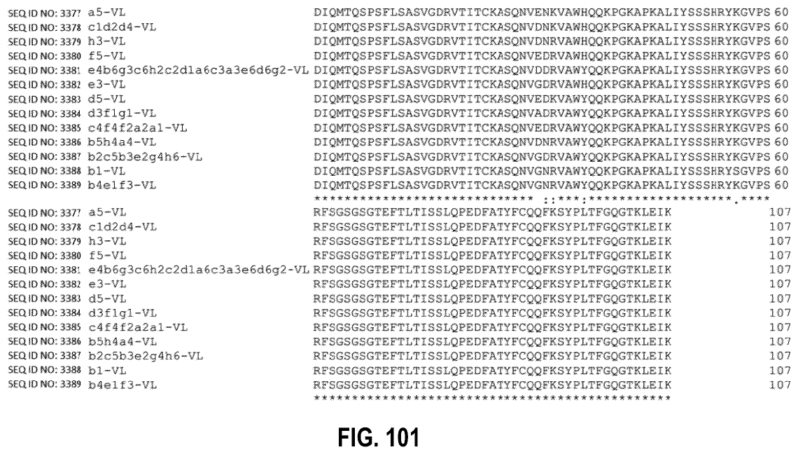

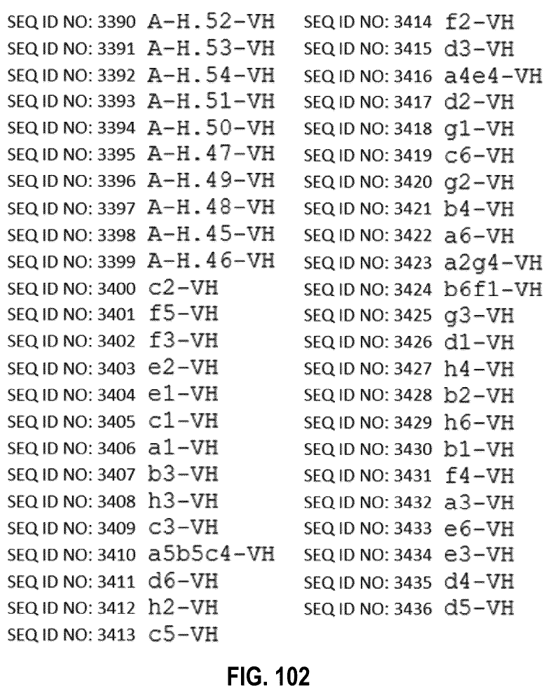

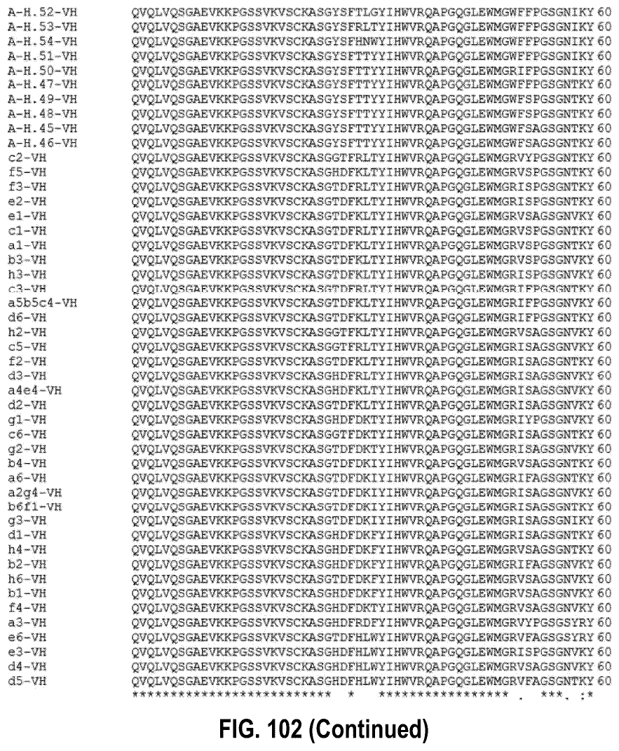

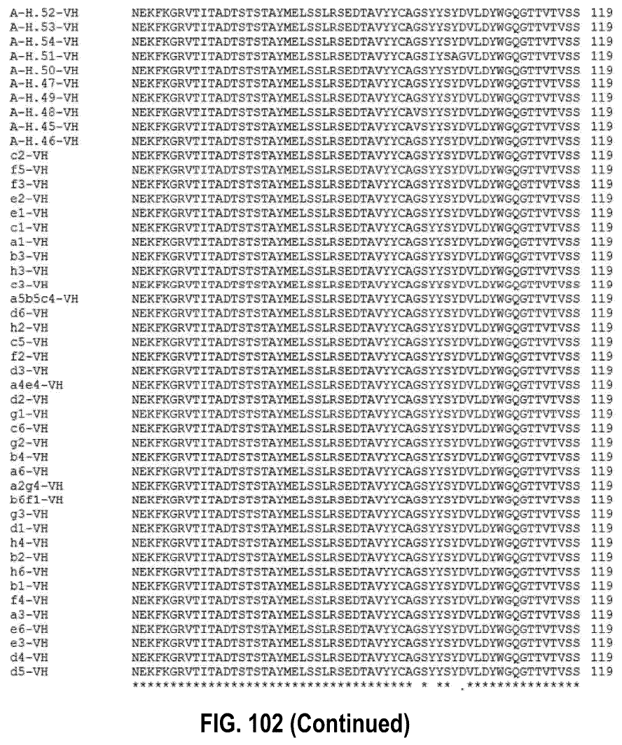

This Example describes the pharmacokinetic (PK) profile of the multifunctional polypeptide molecule as described herein in mice to guide the dosing and/or schedule treatment decision for the efficacy study. The exemplary study design is shown in FIG. 99. Briefly, on day 0, 6-8 week old female NSG mice are implanted subcutaneously with, e.g., 1×10⁶ Raji leukemia cells. On day 2, mice are humanized by injecting 10×10⁶ human PBMC's via the peritoneal cavity. On day 9, mice are treated with a single dose of the multifunctional polypeptide molecule as described herein intravenously. Serum is harvested from animals by submandibular bleed at 0, 0.5, 1, 6, 24, 48, 72, 96, 148 hours (n=3 per time point). Serum drug concentration is measured by Sandwich ELISA.

The serum half-life of the multifunctional polypeptide molecule as described herein in tumor bearing humanized NSG animals is calculated. This data are expected to allow dose and schedule determination for efficacy studies. Exposures at the dose allows coverage above cellular EC90.

### Example 37: Optimization of the Multifunctional Polypeptide Molecule as Described Herein

The multifunctional polypeptide molecule as described herein is optimized to improve affinity for the human and cyno antigen, improve thermal stability, and remove sequence motifs that might pose chemical stability liabilities. For example, ScFv libraries for TCRβV-binding moieties are built using random mutagenesis (Caldwell et al. (1992) Randomization of genes by PCR mutagenesis. PCR Meth. Appl 2:28) or a modified version of Kunkel mutagenesis (Kunkel TA. (1985) Rapid and efficient site-specific mutagenesis without phenotypic selection. PNAS 82(2): 488-92). For affinity improvement, library selections vs human and cyno antigens are performed using standard phage display (Lee, C M et al. (2007) Selection of human antibody fragments by phage display. Nature protocols 2, 3001) and yeast display techniques (Chao G, et al. (2006) Isolating and engineering human antibodies using yeast surface display. Nature Protocols. 1(2):755-69). Thermal challenge of phage or yeast populations is used to select for clones with improved thermal stability. Selections are followed by standard screening methods such as ELISA and flow cytometry to identify individual clones with improved properties. Following hit sequencing and analysis of mutation-activity correlation, second-generation libraries are constructed using the same methods above. Library selections and individual clone screening are repeated as above with the modification that more stringent conditions are applied to select for clones with maximized activity. Following hit sequencing, scFv genes for TCRβV-binding moieties are reformatted into the biologically relevant antibody format for expression, purification, and triaging.

### Example 38: Therapeutic Efficacy of the Multifunctional Polypeptide Molecule as Described Herein in Subcutaneous Human Tumor Xenograft Models

This Example demonstrates the in vivo efficacy of the multifunctional polypeptide molecule as described herein in a subcutaneous human tumor animal model.

On day 1 of the study, e.g., 1×10⁶ cells of the human cancer cell line Raji, stably expressing firefly luciferase (Raji-luc) are subcutaneously injected in the right dorsal flank of female NOD/SCID/IL-2Rγnull (NSG) mice. On day 3, e.g., 10×10⁶ human PBMCs are transplanted into mice by injection into the peritoneal cavity.

Treatment with the multifunctional polypeptide molecule as described herein starts at day 10, when tumors has reached a mean tumor volume (TV) of e.g., 80 mm³. Mean TV of each group is not statistically different from any other group at start of treatment. Mice are treated with e.g., 0.2 mg/kg, 1 mg/kg and 5 mg/kg of the multifunctional polypeptide molecule as described herein every three days for a total of, e.g., 7 doses by intravenous bolus injection. Tumor volume (TV) is measured every 3 days by calipers and progress evaluated by intergroup comparison of TV. Tumor growth inhibition T/C [%] is calculated as T/C [%]=100×(mean TV of analyzed group)/(mean TV of vehicle group). Treatment with the multifunctional polypeptide molecule as described herein is expected to inhibit tumor growth compared to vehicle control treatment. The results are expected to demonstrate that the multifunctional polypeptide molecule as described herein inhibits tumor growth and has anti-tumor activity.

### Example 39: Therapeutic Efficacy of Multifunctional Polypeptide Molecule as Described Herein in Human Tumor Xenograft Models

This Example demonstrates the in vivo efficacy of the multifunctional polypeptide molecule as described herein in a xenograft animal model.

On day 1 of the study, e.g., 10×10⁶ human PBMCs are transplanted into NOD/SCID/IL-2Rγnull (NSG) mice by injection into the peritoneal cavity. On day 7, e.g., 1×10⁶ cells of the human cancer cell line Raji, stably expressing firefly luciferase (Raji-luc) are intravenously injected into NOD/SCID/IL-2Rγnull (NSG) mice. Control animals are injected with e.g., 10×10⁶ cells of the human control cancer cell line K562 stably expressing firefly luciferase (K562-luc). These animals are used to assess specific killing ability of the multifunctional polypeptide molecule as described herein. Treatment with the multifunctional polypeptide molecule as described herein starts at day 16, when tumor engraftment has reached a mean bioluminescence flux level of e.g., 4×10⁷ photons/s. Mean Flux level of each group is not statistically different from any other group at start of treatment. Mice are treated with e.g., 1 mg/kg and 5 mg/kg of the multifunctional polypeptide molecule as described herein every three days for a total of, e.g., 6 doses by intravenous bolus injection. Tumor burden is measured weekly by bioluminescence imaging and progress evaluated by intergroup comparison of total bioluminescence flux (Total Flux). Tumor growth inhibition T/C [%] is calculated as T/C [%]=100×(mean Total Flux of analyzed group)/(mean Total Flux of vehicle group).

The results are expected to demonstrate that the multifunctional polypeptide molecule as described herein inhibits tumor growth and has anti-tumor activity.

### Example 40: Therapeutic Efficacy of the Multifunctional Polypeptide Molecule as Described Herein in Human Tumor Xenograft Models

This Example demonstrates the in vivo efficacy of the multifunctional polypeptide molecule as described herein in a xenograft animal model.

On day 1, e.g., 20×10⁶ cells of the human cancer cell line RPMI-8226, stably expressing firefly luciferase (RPMI-8226-luc) are intravenously injected into NOD/SCID/IL-2Rγnull (NSG) mice. On day 11, e.g., 10×10⁶ human PBMCs are transplanted into mice by injection into the peritoneal cavity. Treatment with the multifunctional polypeptide molecule as described herein starts at, e.g., day 17, when tumor engraftment has reached a mean bioluminescence flux level of, e.g., 4×10⁷ photons/s. Mice are treated with e.g., 0.5 mg/kg of the multifunctional polypeptide molecule as described herein once a week for a total of, e.g., 2 doses by intravenous bolus injection.

Tumor burden is measured weekly by bioluminescence imaging and progress evaluated by intergroup comparison of total bioluminescence flux (Total Flux). Tumor growth inhibition T/C [%] is calculated as T/C [%]=100×(mean Total Flux of analyzed group)/(mean Total Flux of vehicle group).

Treatment with the multifunctional polypeptide molecule as described herein is expected to inhibit tumor growth compared to vehicle control treatment. The results are expected to demonstrate that the multifunctional polypeptide molecule as described herein inhibits tumor growth and has anti-tumor activity.

### Example 41: Production of Bispecifics

All DNA sequences encoding the bispecific molecules were synthesized by GeneArt and cloned into the mammalian expression vector pcDNA3.4. The ExpiCHO expression system was used for expression of BKM0186. ExpiCHO-S Cells were grown to the optimal transfection density and viability in ExpiCHO expression medium according to the manufacturer's protocol. Cells were transiently transfected using the ExpiFectamine™ CHO Transfection Kit according to the manufacturer's protocol. Each chain was added in a 1:1:1 wt/wt/wt ratio. The max titer protocol was used where on the day after transfection, ExpiFectamine™ CHO Enhancer and ExpiCHO™ Feed are added to the flask followed by transfer of the flask to a 32° C. incubator with a humidified atmosphere of 5% CO2 in air. On Day 5 after transfection, the second volume of feed is added and the flask is returned to the 32° C./5% CO2 incubator and harvested on Day 14 after transfection Cells are harvested by centrifugation and subsequent filtration using a 0.22 μm filter.

Following harvest and filtration, the clarified cell culture supernatant was loaded onto a 40 mL column packed with MabSelect SuRe resin equilibrated with Dulbecco's PBS pH 7.4 at a flow velocity of 226 cm/hr using an AKTA Pure FPLC. The column was washed with 20 column volumes (CV) of DPBS or until the UV280 reaches baseline. BKM0186 was then eluted with S column volumes of 20 mM Citrate, 150 mM NaCl pH 3.0. The eluate was neutralized with 10% Tris-HCl pH 8.0. The concentration of the neutralized eluate was quantified using a Nanodrop One-C using the extinction coefficient. Following analysis, the Protein A eluate was diluted 10-fold in cation exchange (CEX) Buffer A: 50 mM MES. 20 mM NaCl pH 5.6 and applied to a Mono S™ 10/100 GL column equilibrated with Buffer A at 458 cm/h using an AKTA Pure FPLC. Following a 10CV wash step with Buffer A, bispecifics were eluted with a 20CV 0-50% gradient of Buffer B: 50 mM MES, 1 M NaCl, pH 5.6. The main peak containing the protein of interest (POI) was pooled. The CEX pool was buffer exchanged using a HiPrep 26/10 desalting column equilibrated with formulation buffer: 20 mM Histidine. 7% Sucrose. 0.02% Tween 80, pH 6.0, at a flow velocity of 113 cm/hr on an AKTA Pure FPLC. The formulated material was quantified and analyzed via analytical SEC (aSEC) and SDS-PAGE to determine purity.

Analytical SEC was performed using an AdvanceBio SEC 300A 2.7 um 4.6×300 mm SEC column equilibrated with 50 mM Sodium Phosphate, 300 mM NaCl pH7.0 at 0.35 mL/min on an Agilent 1100 HPLC. The SDS-PAGE gel was stained with Coomassie Blue stain.

### Example 42: Plasmon Resonance (SPR) Binding

All interactions between bispecifics and relevant receptors were analyzed by Surface Plasmon Resonance (SPR) on a Biacore T200 instrument.

BKM0186 at 2 ug/mL was immobilized on a Series S CM5 chip via human Fc antibody to 80 RU Human IL2Ra and cynomolgus IL2Ra were diluted to 500 nM, human IL2R beta-gamma and cynomolgus IL2R beta-gamma were diluted to 125 nM, and human IL2R trimeric complex was diluted to 50 nM and then serially diluted two-fold in 1×HBS-EP+ buffer for a total of 10 concentrations. Multi cycle kinetics was run where the chip is regenerated with 3 M Magnesium Chloride between each cycle followed by a new injection of BKM0186. An association time of 120 seconds and a dissociation time of 150 seconds for human and cyno IL2Ra. 300 seconds for human and cyno IL2R beta-gamma, and 900 seconds for the human IL2R trimeric complex was done at 30 uL/min. This assay was run in 1×HBS-EP+ Buffer pH 7.4 and 25C. Sensorgrams were corrected by double reference subtraction using the reference flow cell not treated with BKM0186 and a blank cycle of buffer alone. BIAevaluation software was used for data analysis and the data were fit using a 1:1 Langmuir binding model for calculation of the KD value (Koff/Kon). Due to faster off rates observed for human and cyno IL2Ra binding, the data were fit using a steady state model where equilibrium binding response is analyzed.

BKM0186 at 2 ug/mL was immobilized on a Series S CM5 chip via human Fc antibody to 80 RU. Human Vβ6 TCR and cynomolgus Vβ6 TCR were diluted to 250 nM and then serially diluted two-fold in Ix HBS-EP+ buffer for a total of 10 concentrations. Multi cycle kinetics was done with an association time of 120 seconds and a dissociation time of 600 seconds, for human Vβ6 TCR and 300 seconds for cynomolgus Vβ6 TCR at 30 uL/min. This assay was run in 1×HBS-EP+ Buffer pH 7.4 at 25C. Sensorgrams were corrected by reference subtraction using the reference flow cell not treated with BKM0186. BIAevaluation software was used for data analysis and the data were fit using a 1:1 Langmuir binding model for calculation of the K_{D} value (K_{off}/Kₒₙ).

BKM0186 has high affinity binding (Table 19) to both human and cynomolgus Vβ6 TCRs as assessed by surface plasmon resonance (SPR). Additionally, BKM0186 exhibits comparable binding to both human and cynomolgus IL-2Rα and IL-2Rβγ heterodimers and the binding affinities are broadly in line with previously reported values.

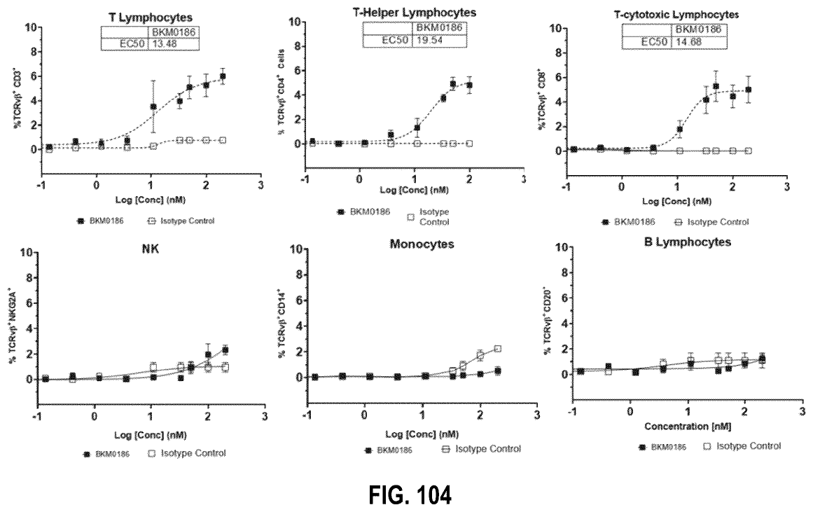

Binding of BKM0186 to T cells as assessed using FACS analysis of PBMC's further demonstrates exquisite selectivity of BKM0186 to Vβ6 T cells with no measurable binding observed to any other immune cells (FIG. 104).

TABLE 19
Binding affinity of BKM0186 to the cognate binding receptors
measured using Surface Plasmon Resonance (SPR)

| Antigen | BKM0186 (K_{D}) |
|---|---|
| Human TCRVβ | 1.7 nM |
| Cyno TCRVβ | 4.9 nM |
| Human IL-2Rα | 45 nM |
| Cyno IL-2Rα | 48 nM |
| Human IL-2Rβγ | 3.8 nM |
| Cyno IL-2Rβγ | 3.3 nM |
| Human IL-2Raβγ | 0.1 nM |

### Example 43: Selective Binding of BKM0186 to T Cells Within PBMC Mixture

Normal health donor human PBMCs were resuspended in at 2 million cells/mL in PBS and 100 μL added to each well of a round bottom tissue culture treated sterile plate. Post staining with Fixed Viability Dye, appropriate quantities of commercially available fluorochrome labeled surface antibodies (Panel 1) were added to the appropriate sample wells. The plates were vortexed and incubated at 2-8° C. (refrigerated) in the dark for 30 minutes. Following staining for Panel 1, plates were centrifuged (set at 400×g; 5 minutes; RT), supernatant aspirated and washed twice with 200 μLs of 1% BSA PBS. Cell pellets for Panel 1 were then resuspended in 0.2 mL of 1% PFA, before transfer to a 96 well u-bottom plate for analysis.

TABLE 20
Exemplary antibodies used

| # | Antibody | Conjugate | Clone | Vendor |
|---|---|---|---|---|
| 1 | CD4 | BV7185 | L200 | BD |
| 2 | CD8 | BV650 | RPA-T8 | BD |
| 3 | CD2 | Per Cp Cy5.5 | RPA-2.10 | BioLegend |
| 4 | CD3 | BV605 | 10D12 | Miltenyi |
| 5 | NKG2A | PE | Z199 | Beckman Coulter |
| 6 | TCRvβ | AF647 (Read as APC) | — | Marengo |
| 7 | CD20 | PE Cy7 | J3.119 | Beckman Coulter |
| 8 | CD14 | FITC | M5E2 | BioLegend |
| 9 | Live/Dead | NUV450 | N/A | ThermoFisher |

Binding of BKM0186 to T cells as assessed using FACS analysis of PBMCs further demonstrates exquisite selectivity of BKM0186 to Vβ6 T cells with no measurable binding observed to any other immune cells (FIG. 104).

### Example 44: Binding of BKM0186 to Pure T Cells Expressing Either Vβ6 TCR and/or CD25

Pan T-cells were isolated from PBMC's via negative selection on the autoMACS. T-cells were stained for TCRVβ6-5 and sorted on the Sony SH800 cell sorter into cell culture media. Sorted TCRVβ6-5 T-cells and non-sorted pan T-cells were activated and expanded using anti-CD3/CD28 beads followed by IL-2 culture. Portion of activated T-cells were allowed to rest for 3-days in serum-free media. Anti-CD25 FACS staining to confirm CD25 upregulation (from activation) and downmodulation (from resting). T-cells that represents TCRVβ6-5 (pos) CD25 (hi); TCRVβ6-5 (pos) CD25 (low); TCRVβ6-5 (neg) CD25 (hi): TCRVβ6-5 (neg) CD25 (low) are checked for BKM0186 construct binding.

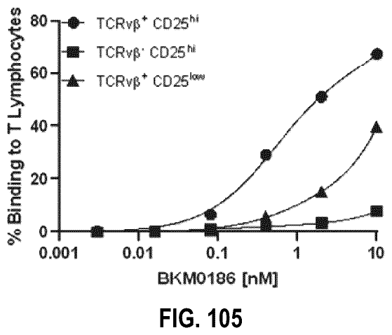

As shown in FIG. 105, BKM0186 was shown to bind Vβ6 CD25H T cells (circle) with greater avidity, as a result of co-operative binding, with a binding ECso of 0.5 nM compared to either non-Vβ6 CD25^{Hi} T cells (square) and Vβ6 CD25^{Low} T cells (triangle). This avidity effect confirms that BKM0186 engages both Vβ6 and IL-2R via a cis binding mode.

### Example 45: In-Vitro T-Cell Stimulation and Expansion in Primary Human Peripheral Blood Mononuclear Cells (PBMCs)

Peripheral blood mononuclear cells were isolated from blood leukapheresis samples using density gradient separation. Human T-cells were stimulated and expanded from peripheral blood mononuclear cells with the addition of BKM0186 (concentration specified in text) cultured in X-Vivo 15 media for 5 days, incubated at 37° C.

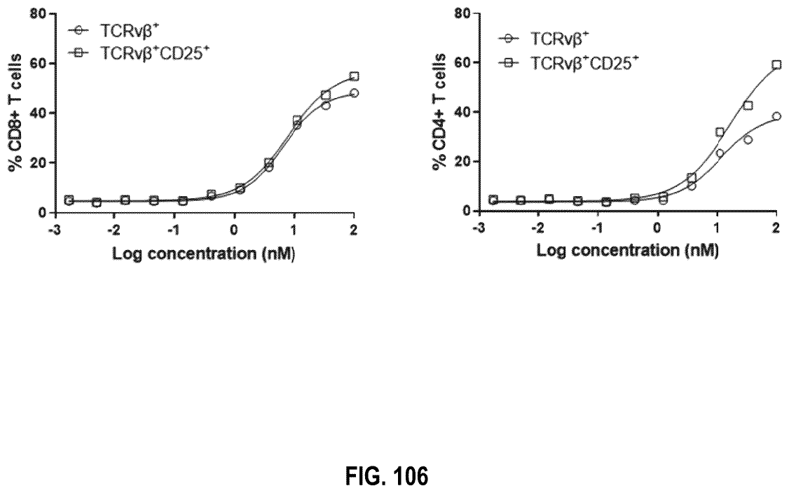

Binding of BKM0186 to Vβ6 T cells results in selective expansion of only Vβ6 T cells when assessed over a period of 5 days in healthy human PBMCs at 37° C. FIG. 106 shows a concentration-effect curve of BKM0186-mediated activation (CD25 expression) and expansion (positivity for Vβ6 TCRs) in both human CD4⁺ and CD8⁺ T cells. These plots show the extent of proliferation of Vβ6 T cells as a proportion of the total CD8⁺ and CD4⁺ T cell populations with an EC₅₀ of 6 and 12 nM, respectively. Pronounced upregulation of CD25 in the expanded CD4⁺ and CD8⁺ Vβ6 T cells indicates the activated state of these cells Additional in vitro activation assays in human PBMCs demonstrate upregulation of other activation markers (e.g., Granzyme B, CD69. ICOS, etc) particularly on CD8⁺ T cells, further characterizing their cytotoxic potential.

Expanded CD4⁺ and CD8⁺ Vβ6 T cells were further assessed for various markers of memory sub-pools that demonstrated a consistent shift of BKM0186-treated human PBMCs to either central memory (TCM) phenotypes.

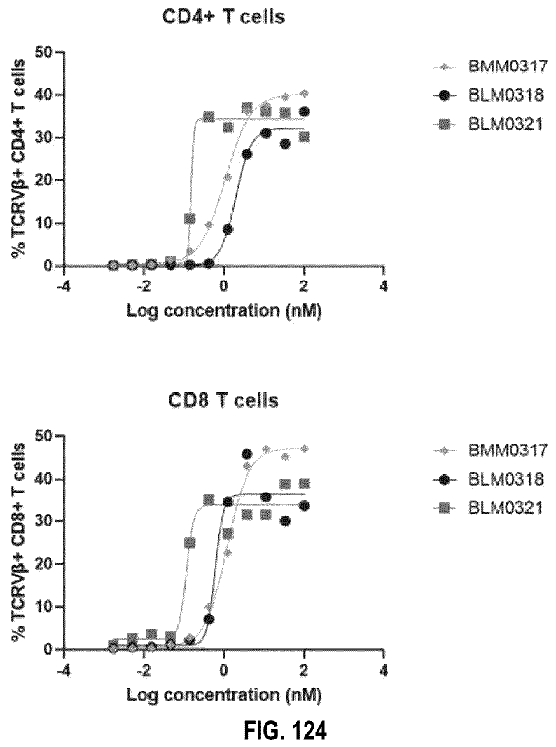

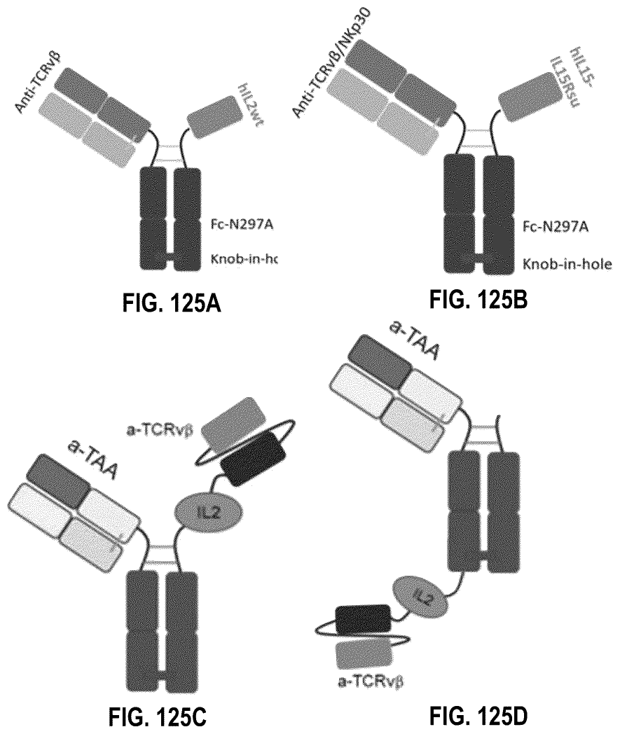

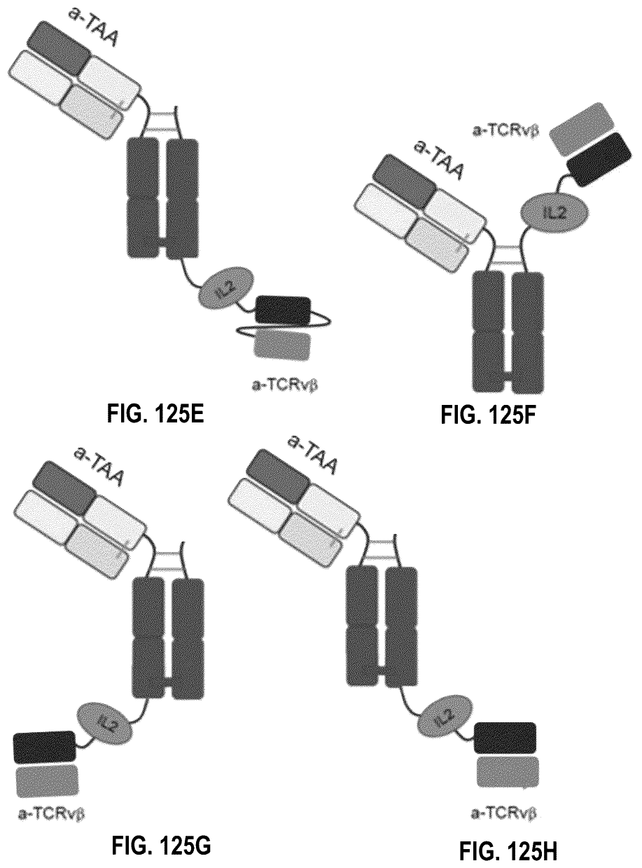

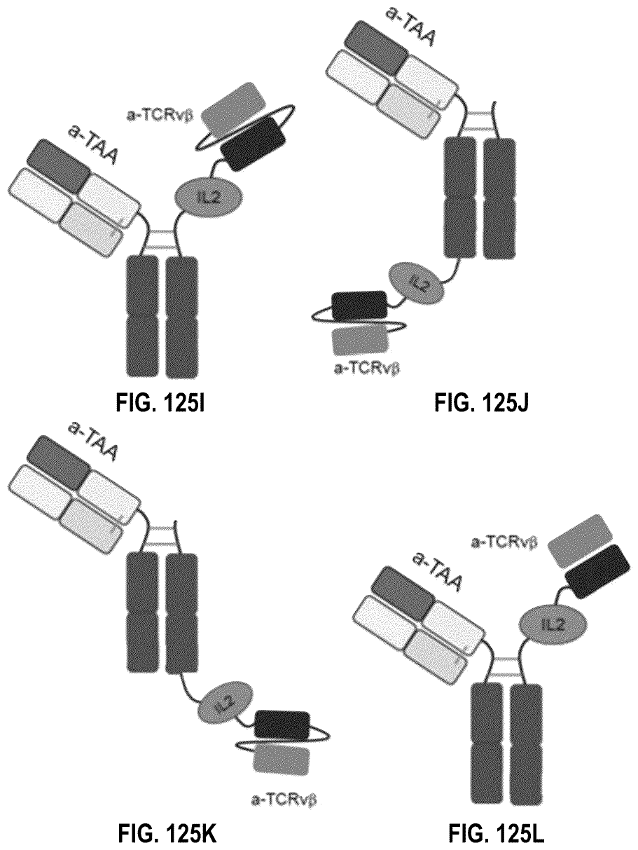

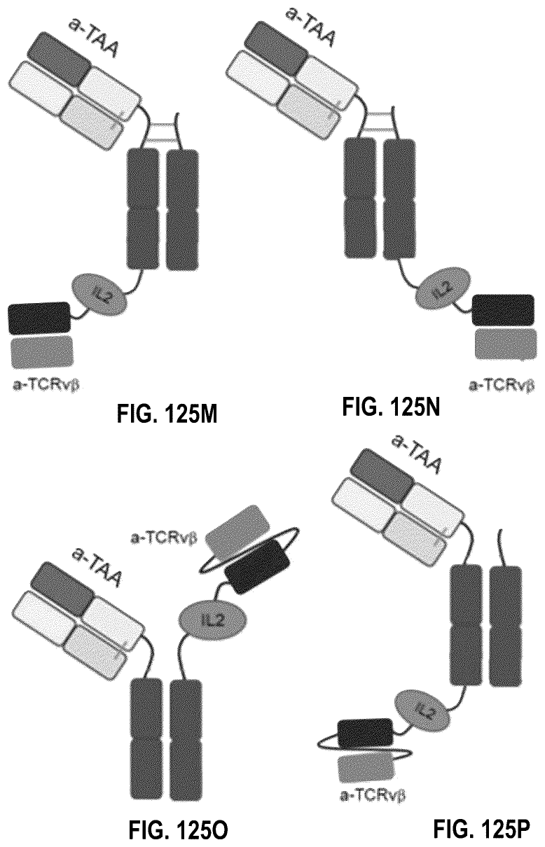

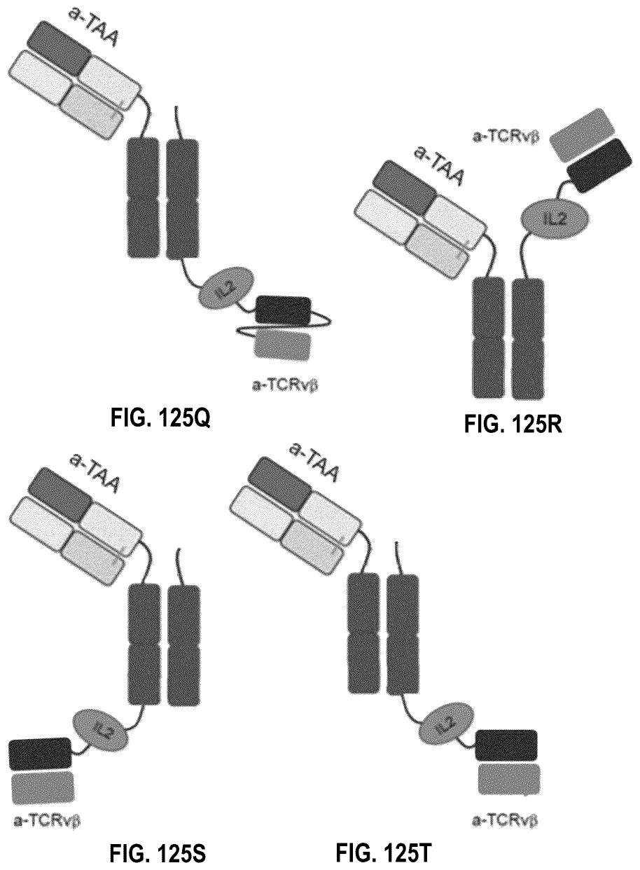

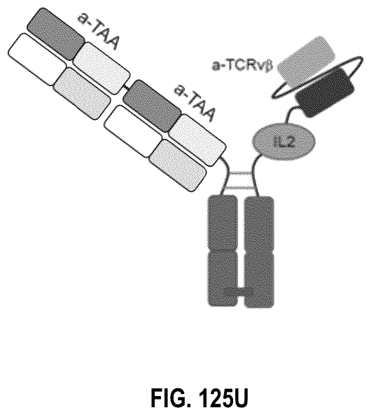

Using identical protocols, expansion of TCRVβ+ T cells mediated by additional bispecific constructs BMM0317. BLM0318 and BLM0321 was demonstrated as shown in FIG. 124.

### Example 46: In-Vitro TCR Sequencing

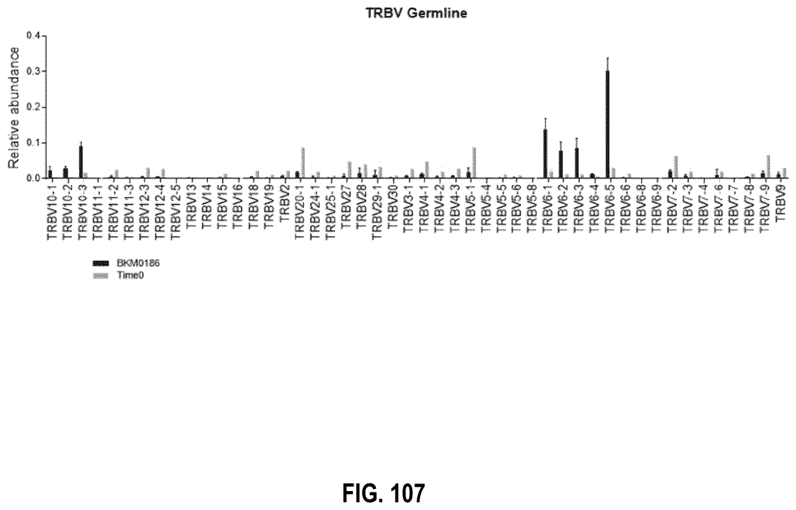

Total RNA was extracted from T cells using Maxwell Simply RNA Kit. The RNA was quantified using Qubit High Sensitivity RNA Assay and quality was analyzed using Agilent Tape station. Sequencing libraries were generated using SMARTer Human TCR a/b Profiling Kit for human cells, or SMARTer Mouse TCR a/b Profiling Kit according to the manufacturer's protocol. Final libraries were then pooled and sequenced (paired end. 300 bp) on Illumina MiSeq. Data generated was demultiplexed and FastQC was performed after trimming. MiXCR pipeline tool was used to specifically align sequence reads to TCR germline segments of TRA. TRB. TRD and TRG genes in the datasets for clonotype identification, CDR3 sequences and clonotype abundances. TRBV genes are counted and grouped together among their specific clonotypes and plotted as a bar chart representing frequency/abundance. Each TRBV gene is plotted against relative abundance (where 1 equals 100% of total TRBV gene), equivalent to total T-cell abundance BKM0186 selectively expanded T cells bearing TRBV6-1, TRBV6-2, TRBV6-3, TRBV6-5 and TRBV10-3. (FIG. 107)

### Example 47: Expansion and Activation of TCRVβ6+ T Cells by BKM0186

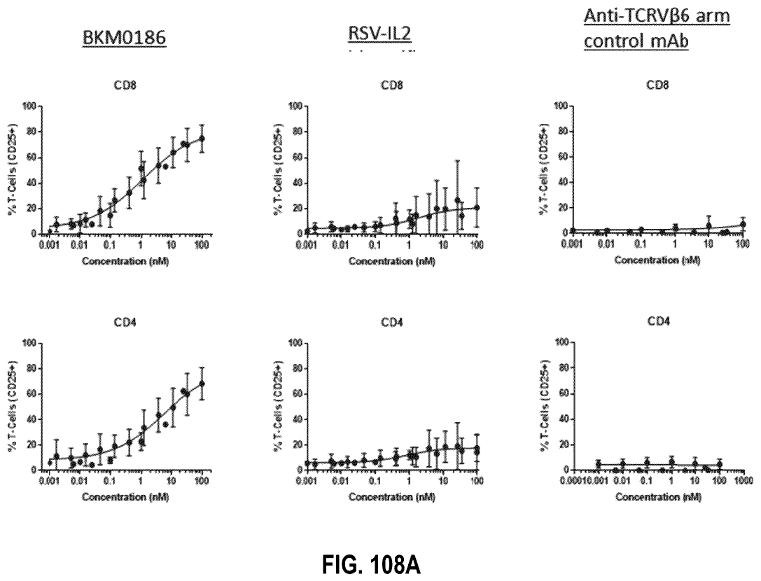

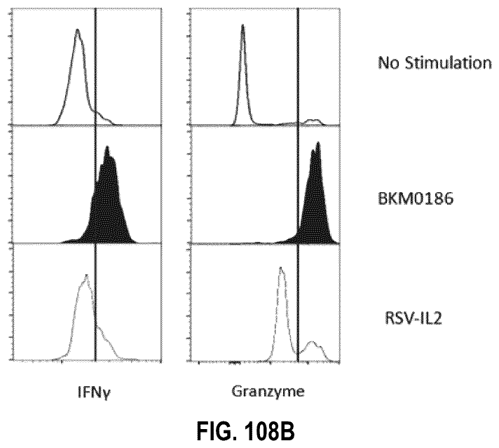

Cryo-preserved PBMCs were thawed and rested for 30 min at 37° C., then plated at 3×10⁵ cells per well in a 96-well round-bottom plate. Sterile biotinylated constructs were diluted in X-Vivo 15 media and serial dilutions were performed. Wells without construct (media alone) were used to determine background signal. Cells were incubated with construct for 5 days at 37° C. Early on day 5. Brefeldin was added to all wells and cells were incubated for another 4 hours. Cells were then transferred to a V-bottom plate for staining. Cells were spun and washed, then stained with viability dye, followed by surface markers, biotinylated TCRvB monoclonal antibody, and secondary streptavidin antibody to detect both TCRvB-bound cells and construct. After thorough washing, cells were fixed and permeabilized using the eBioscience FoxP3 staining kit. FoxP3, granzyme B, and IFNg were stained in permeabilization buffer. Following washes with permeabilization buffer, cells were resuspended in flow buffer (PBS+BSA) and run on the Cytek Aurora. Analysis was performed in FlowJo. FIG. 108A shows robust expansion of both CD4⁺ TCRVβ6+ and CD8⁺ TCRVβ6+ T cells mediated by BKM0186 while very little to no expansion was observed with the controls RSV-IL2 bispecific and anti-TCRVβ6 arm mAb indicating that the expansion is mediated by crosslinking of both TCR and IL2R. The activation of TCRVβ6+ T cells by BKM0186 resulted in upregulation of granzyme B and Interferon gamma cytokine as shown in FIG. 108B.

### Example 48: Evaluation of Memory T Cell Differentiation

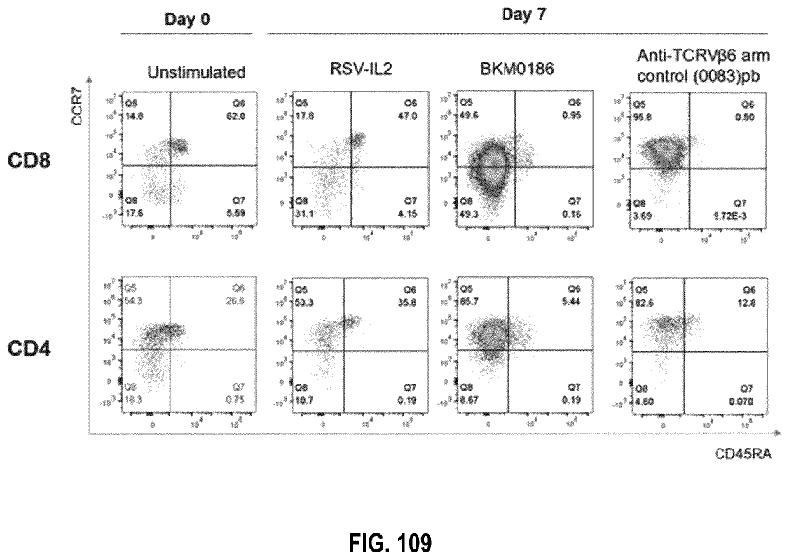

Peripheral blood mononuclear cells, PBMC's were isolated from blood leukapheresis samples using density gradient separation. Human T-cells were stimulated and expanded from peripheral blood mononuclear cells with the addition of BKM0186 or control RSV-IL2 at 10 ng/ml in solution and cultured in X-Vivo 15 media for 7 days, incubated at 37° C. Monovalent anti-TCRVβ6 antibody was immobilized on to plate for stimulation to serve as a TCRVβ-only control. After stimulation, PBMCs were stained for CD3, CD4, CD8. CD45RA, CCR7, and TCRVβ6 for flow cytometry analysis using (FITC) anti-human CD45RA (clone 5H9), (PE) anti-human CCR7 (clone OX-108), (BV-421) anti-human CD4 (clone OKT4), (PerCP-Cy 5.5) anti-human CD8 (clone SKI). Biotinylated TCRVβ6 monoclonal antibody, AlexaFluor 647 Streptavidin antibody. TCRVβ6 positively stained CD4 or CD8 T cells were gated to characterize memory phenotype expression mediated by BKM0186 and control treatment. As shown in FIG. 109, BKM0186 showed robust expansion in both Central memory (upper left) and Effector memory (lower left) quadrants.

### Example 49: Cytokine Induction Mediated by Stimulation of PBMCs by BKM0186

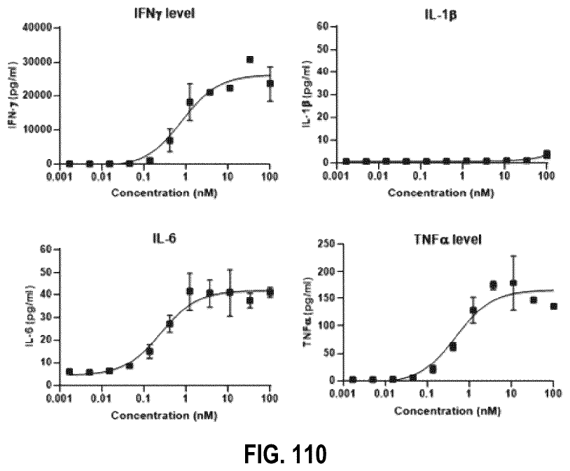

Supernatants from in vitro human PBMC stimulation assays were further assayed for inflammatory cytokine levels using Meso Scale Detection (MSD) immunoassays. Stimulation of human PBMCs with BKM0186 led to moderate release of some proinflammatory cytokines as shown in FIG. 110. Consistent with published findings, these cytokine levels were generally lower than levels as saved from human PBMCs stimulated with an anti-CD3 bispecific antibodies. Of note, the cytokine release profiles following activation of cynomolgus monkey PBMCs with BKM0186 was similar to the profile in stimulated human PBMCs using this assay.

### Example 50: Binding Ex Vivo Cytotoxicity Evaluation of BKM0186 in Autologous TIL-PDX-O Co-Culture Using High Content Analysis (HCA)

BKM0186 within four concentrations ranging from 0.3-10 μg/mL as a single agent, was evaluated ex vivo in autologous TIL-PDX-O co-culture from 4 patient-derived Champions TumorGraft® models: CTG-3493, CTG-3571 representing human non-small cell lung cancer, CTG-3629 representing human rectal cancer, and CTG-3631 representing human colorectal cancer by confocal high content analysis (HCA). Tumor cell cytotoxicity was also assayed by the relative organoid fluorescent intensity and relative organoid area.

The high content analysis assay using confocal microscopy was developed with three colors. TILs and PDX-O tumor cells are labeled with distinct dyes prior to co-culture, and a dead cell detection dye is added to the co-culture at the study endpoint. On Day-3, cryopreserved PDX fragments were thawed, counted, and stained with CellTracker Deep Red (CTDR) Stained PDX-fragments were washed and re-suspended in PDX-organoid media and plated at a density of approximately 5000 cells in 50 μl organoid media/well in a 96-well plate ultra-low attachment round bottom plate. Organoids were allowed to form over a period of 3-days. After three days, on Day 0, PDX-O matched TILs were stained with Cell Trace Violet (CTV). Stained TILs were washed, re-suspended in TIL media, and co-cultured with matched PDX-O at a density of approximately 25,000 cells/well (in 50 μL. TIL media) in the appropriate wells. On the same day (Day 0), control(s) and BKM0186 at different concentrations (in additional 50 μL PDX-O media) were administered in quadruplicates. At the study endpoint, on Day 5 (approx. 120 hours after TIL and test agent/control administration), the NucGreen® Dead 488 probe was added directly to the wells to stain dead cells. Image z-stacks were captured approximately 3 hours after dead cell stain at 4× magnification using CellInsight CX7 LZR HCA instrument. Images were collected using filter sets appropriate for each marker and analyzed using HCS Cellomics Studio Software (per well). For all the models examined. PDX-O cytotoxicity was quantified through examination of organoid fluorescent intensity and is reported as relative organoid fluorescent intensity and relative organoid area (normalized to Isotype molecule).

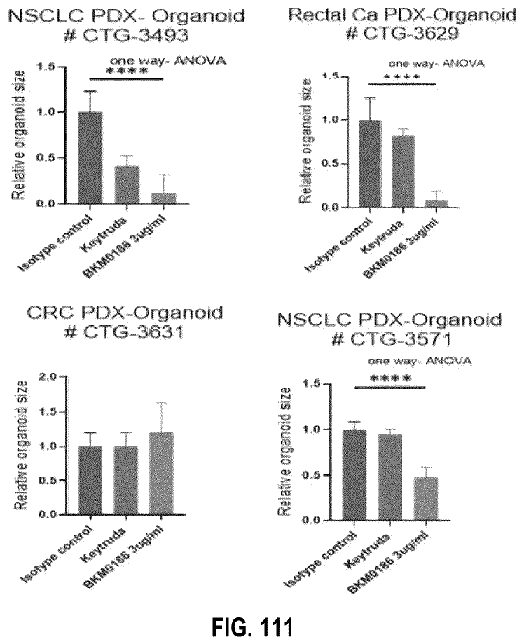

In 3 out of 4 donors, BKM0186 potently expanded and activated in situ human TILs that then elicited potent killing of surrounding tumor tissue as represented by reduction in the organoid size (FIG. 111). Both TIL expansion and human tumor-killing was significantly more potent with samples treated with BKM0186 compared with the same samples treated with a clinically available anti PD-1 antibody (Pembrolizumab: Keytruda).

### Example 51: In Vivo Mouse Model Experiments (Syngeneic Mouse Tumor Studies)

As a result of the limited homology between human and mouse or other rodent species at the TRB locus, a direct homolog of the Vβ6 gene (TRBV6) does not exist and so the likelihood of species cross-reactivity of BKM0186 and BKM0281 beyond non-human primates is very low. Therefore, efficacy experiments performed in syngeneic murine tumor models were conducted with a mouse orthologue molecules (mBKM0186 and mBKM0281) with similar molecular configurations as human molecules, that targets and expands Vβ13 TCR expressing murine T cells and recapitulates the immunology of BKM0186 and BKM0281 in mice. Vβ13 T cells are one of the most abundant germline Vβ TCR variants in mice and are expressed in TILs of all tumor bearing mice.

All mice were acquired from The Jackson Laboratory. All procedures were performed in compliance with established ethical regulations and were approved by the Institutional Animal Care and Use Committee (IACUC).

Mouse tumor cell lines RENCA (CRL-2947), B16F10 (CRL-6475), CT26 (CRL-2638), RM1 (CRL-3310) and EMT6 (CRL-2755) were obtained from American Type Culture Collection. MC38 were obtained from NIH (ENH204-FP). Cell lines were tested for mycoplasma and other pathogens and cultured according to their guidelines.

BALB/c female mice were implanted with 1×10⁵ of RENCA cells resuspended in PBS: 5×10⁴ of CT26 or EMT6 cells resuspended in PBS. C57BL/6 female mice were implanted with 5×10⁴ of MC38; 2×10⁴ of B16F10 cells resuspended in PBS. C57BL/6 male mice were implanted with 2×10⁴ of RM1 cells resuspended in PBS by subcutaneous injection on the right flank. Following tumor implantation, mice were randomized into treatment groups (8-10 mice per group) when tumor size reached between 80-150 mm³. Mice were monitored for morbidity and mortality daily.

Antibodies were administered by intraperitoneal injection (Day 0) with murine surrogate bispecifics (1.0-1.5 mg/kg), or PBS once per week for 3-4 treatments. Tumor growth was monitored over time using caliper measurement of X and Y diameter, and body weight recorded. Tumor volume was calculated [X*Y*(X/2)]. Mice were euthanized when tumor volume reached 2000 mm³, or loss of body weight of more than 20% within a 1-week timeframe as indicated in the IACUC protocol CR-0147. Surviving cured mice will be continued to be monitored until they reach Day 100. Additionally, tumor-free cured mice were rechallenged with tumor cell lines on the left flank to evaluate memory response.

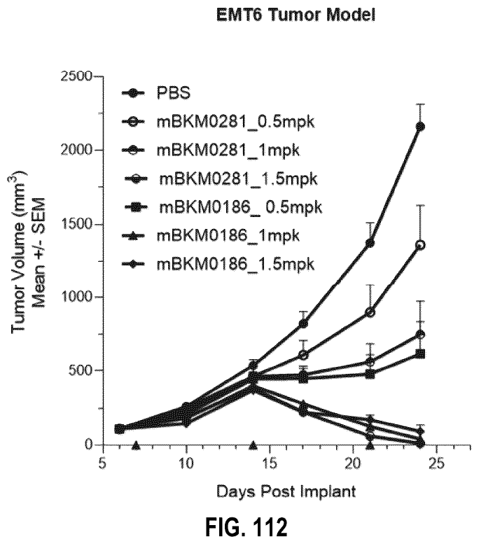

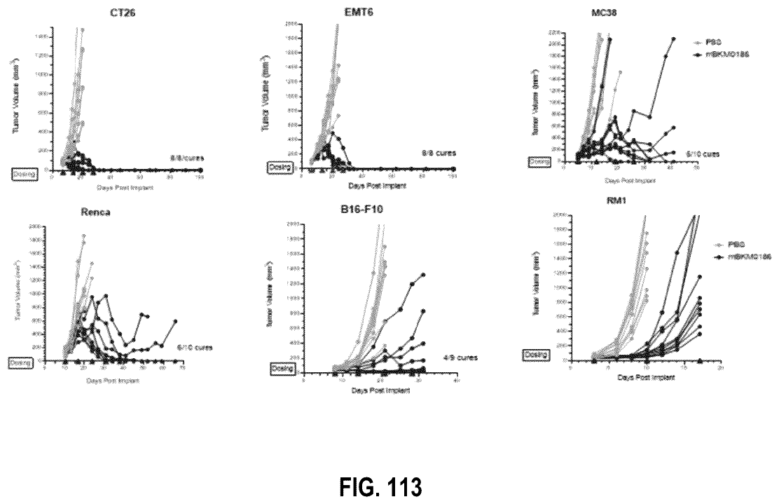

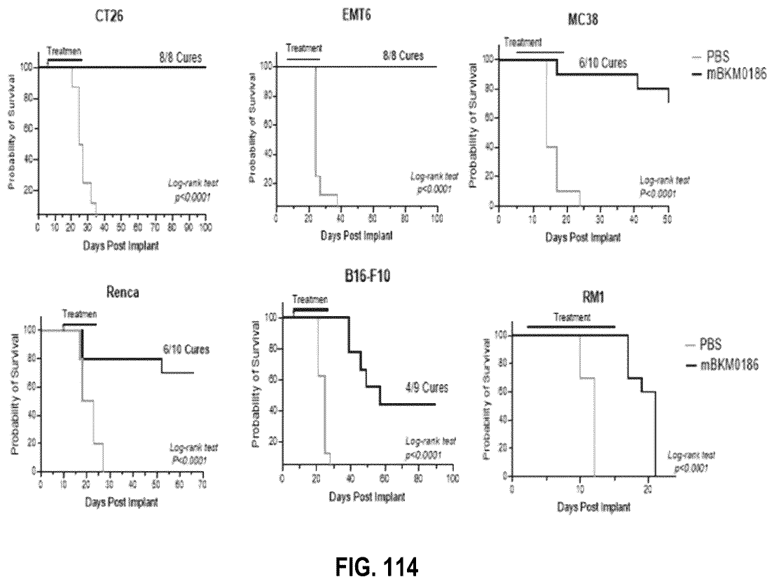

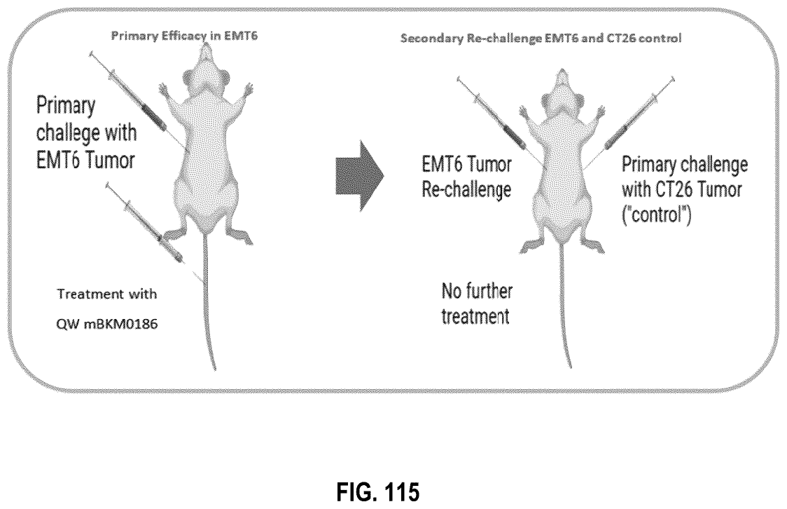

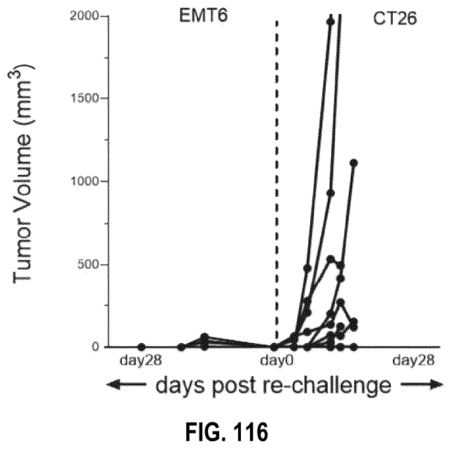

As shown in the Kaplan-Meier survival plots in FIG. 114 and tumor growth curves in FIG. 113, mBKM0186 resulted in significant regression of tumors in all the models including the anti-PD-1 resistant RENCA. B16F10 and RM1 models, resulting in significant improvement in survival, except in the very aggressive RM1 model. In EMT6, this effect was dose related across a dose range of QW IP 0.5, 1, and 1.5 mg/kg doses of mBKM0186 and mBKM0281 with weekly doses of 1 mg/kg and 1.5 mpk of mBKM0186 and 1.5 mpk of mBKM0281 appearing to be optimal (FIG. 112). Furthermore, 100% cured EMT6 mice upon rechallenging with the respective tumor cells exhibited no tumor growth demonstrating induction of memory. This long-term protection from tumor re-challenge in mice also appears to be due to the accumulation of memory CD8+Vβ 13 T cells (FIG. 115 and FIG. 116). Overall, these data support the possibility that BKM0186 will promote both potent and durable anti-tumor responses as a single agent in humans.

### Example 52: Pharmacodynamics and Mechanistic Studies in Syngeneic Models

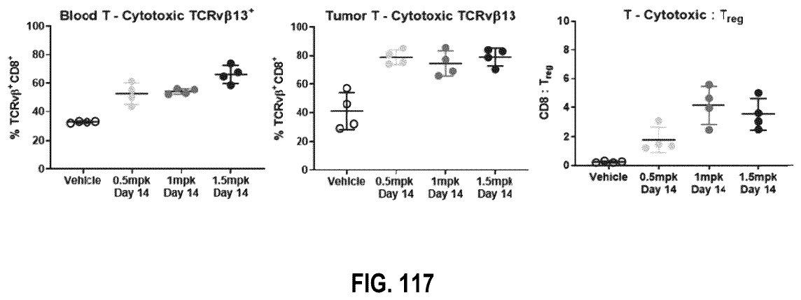

To further explore mechanisms underlying the observed anti-tumor activity with mBKM0186, immune profiling of TILs isolated from murine EMT6 tumors after treatment with vehicle or mBKM0186 were assessed by flow cytometry and immunohistochemistry (IHC) on tissue sections. Following 14 days of QW dosing (i.e., 2 doses) with mBKM0186, a significant accumulation of Vβ CD8⁺ T cells in TILs was noted compared with vehicle treated mice. Given the degree of expansion of Vβ CD8⁺ T cells, this was reflected in an overall significant increase in the numbers of CD8⁺ T cells in tumors with a significant proportion showing upregulation of cytolytic molecules such as granzyme B. (FIG. 117)

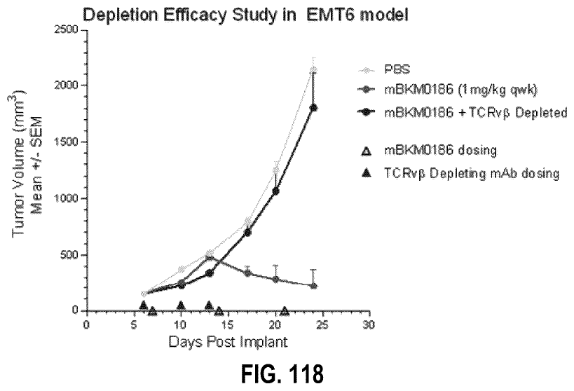

Importantly, the ratio between CD8⁺ T cells and FoxP3⁺ Regulatory T cells (Treg) also significantly increased post treatment with mBKM0186. To confirm that mBKM0186 expanded Vβ CD8⁺ T cells are responsible for the anti-tumor effects observed in syngeneic mice, EMT6 mice were treated with mBKM0186 in the presence and absence of a specific Vβ T cell-depleting antibody. As shown in FIG. 118, the anti-tumor effects of mBKM0186 were abolished in mice that also received doses of the Vβ T cell-depleting antibody, thereby confirming the central importance of the expanded and activated Vβ T cell population to the efficacy of mBKM0186 in mice.

### Example 53: In Vivo Cynomolgus Monkey Experiments

Exploratory studies of intravenous (IV) infusions of BKM0186 and BKM0281 at multiple doses were conducted to assess preliminary safety and tolerability, pharmacokinetics, and pharmacodynamics. All studies were conducted in naïve female cynomolgus monkeys (Macaca fascicularis, of Cambodian origin, that were 2.3-4.9 years old with body weights in the range of 2-4 kg).

For Pharmacokinetic Assessments, concentrations of BKM0186 were measured using the MSD electrochemiluminescence (ECL) assay platform. The assay comprises a plate-bound TCR Vβ6 antigen reagent to capture BKM0186 or BKM0281, followed by a two-step biotinylated anti-human IL-2 antibody and streptavidin-Sulfotag-TAG for the detection of the drug complex, with a validated lower limit of quantitation (LLoQ) of 0.05 nM. In brief, plates were coated with 25 uL/well of TCR Vβ6 antigen, incubated overnight at 4° C., followed by blocked using 250 μL/well of PBS containing 3% BSA for 2 hours. Serum samples were diluted 1:50 in PBS-T to fall in the linear range of the standard curve (samples which generated signals that did not fall within the linear range of the standard curve were re-run at either 1:200 for samples above the linear portion; and 1:20 for samples that fell below the linear portion. Samples were added to the plate at 25 uL/well in duplicates and incubated overnight at 4° C. 25 uL/well of the biotinylated anti-IL2 antibody at a final concentration of 5 ug/mL in PBST+1% BSA were added and incubated at room temperature (RT) for 2 hours. Plates were washed, then a 1:1000 dilution of Streptavidin-Sulfo-TAG in PBST was added to the plates at 25 uL/well for 30 mins at RT, then washed. A 1:4 dilution of MSD Read Buffer A in deionized water was as added at 150 μL/well to plates, that were then read on an MSD plate reader. Standard curves used to calculate analyte concentrations were established by fitting the signals from the standard to a 4-parameter logistic (or sigmoidal dose-response) model with a 1/Y\hat{ }2 weighting. The LLOQs were derived from the lowest concentration of the standard curve that met the following criteria: CV %<20, and Recovery % 85-115.

For Pharmacodynamic Assessments, assessments were undertaken in blood over a range of timepoints. The assessment of Vβ6 CD8+ T cells in blood samples drawn from dosed monkeys was achieved using flow cytometry for the quantification of Vβ6 T cells expansion and activation. T cell activation were measured by the expression of IL 2Rα (CD25) and inflammatory cytokines IFNγ, TNFα, and IL6. For flow cytometry assays, 50 μL of whole blood (for cell surface staining), or 95 μL of whole blood (for intracellular staining) were used per well (96-deep well plate) of staining. Following cell surface staining. 1.5 mL of FACS lysing solution 1× was added to each well and incubated in the dark at RT for 10 minutes. Immediately after the incubation, plates were centrifuged (set at 400×g; 5 minutes: RT), supernatant aspirated and washed with 1700 mL of stain buffer (PBS plus 0.04% BSA). Cell pellets were then resuspended in 0.4 mL of 0.05% formalin solution diluted in FACSFlow and transferred to a 96 well u-bottom plate for analysis. For intracellular staining, prewarmed 1.8 mL of 1×LYSE/FIX buffer was added to each well. The plates were placed in an incubator and set to maintain 37° C., for 15 mins and centrifuged (set at 600×g; 8 mins: RT), washed, and resuspended in 1 mL of cold Perm Buffer III for 30 minutes on wet ice. Antibodies were added to the appropriate sample wells for staining. The plates were incubated in a refrigerator set to maintain 4° in the dark for 60-70 minutes. Following the incubation, plates were washed twice (centrifuge set at 600×g; 8 minutes; 4°), supernatant aspirated and cell pellets were then resuspended in 0.3 mL of stain buffer and transferred to a 96 well u-bottom plate for analysis. Samples were analyzed utilizing the LSRFortessa™ II Flow Cytometer and DIVA® 8.0.1 software. Inflammatory cytokines were measured using the Non-Human Primate cytokine kit (Millipore, #PRCYTOMAG-40k), per manufacturer's instructions, and detected using the Luminex BioPlex Systems.

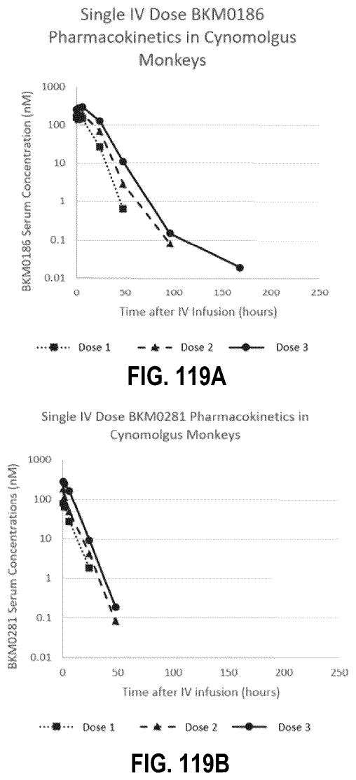

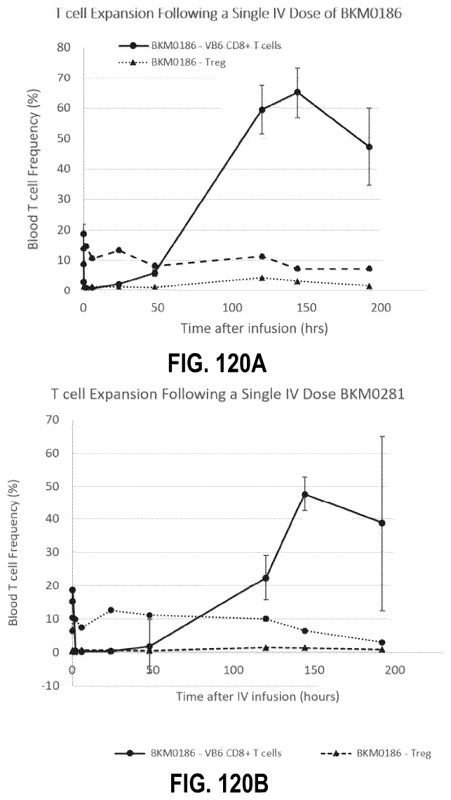

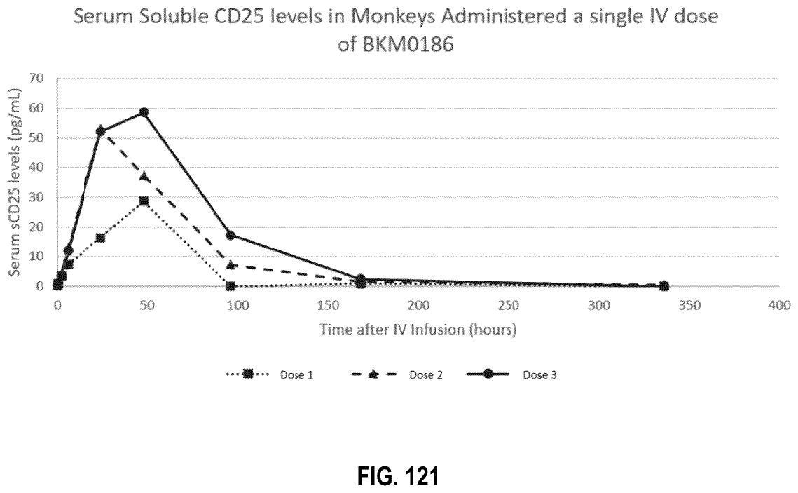

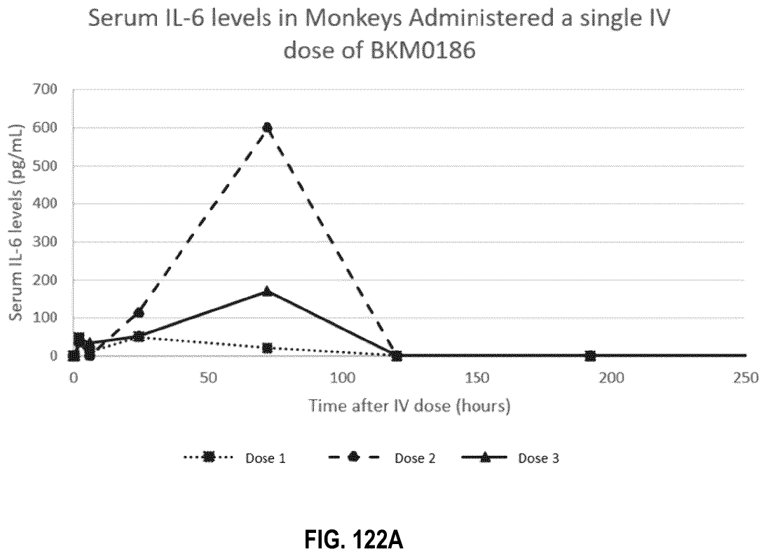

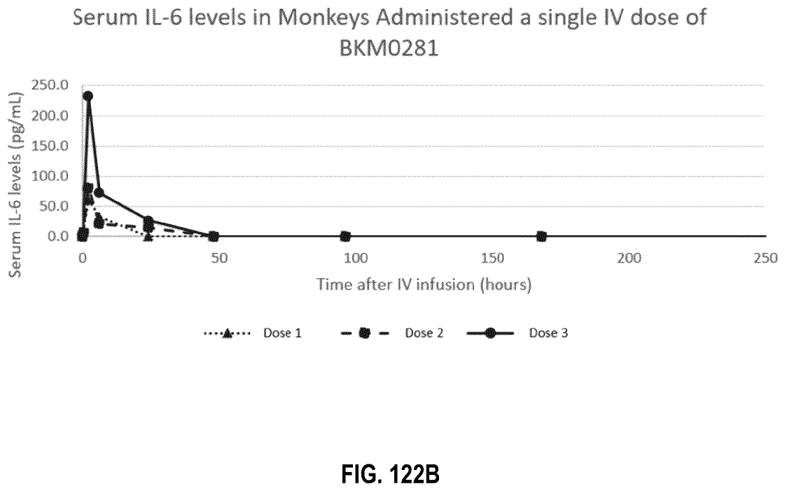

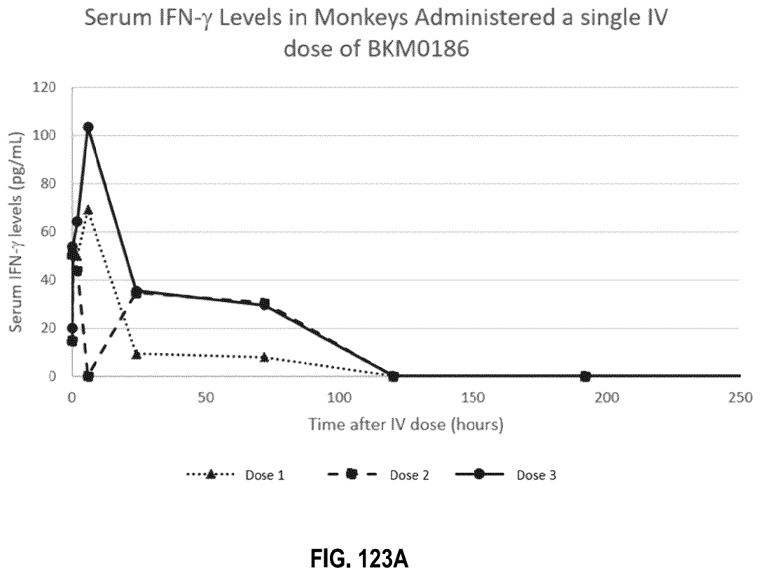

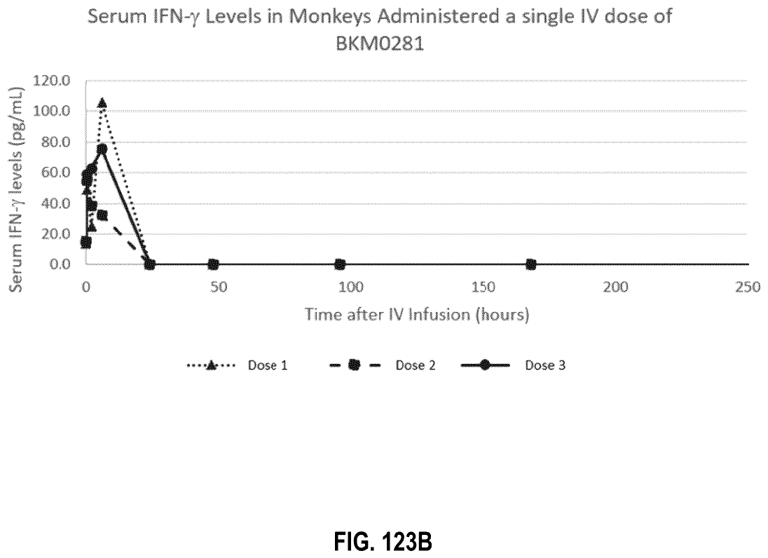

FIG. 119A and FIG. 119B (A&B) illustrate concentration-time curves for all IV doses (30 min infusion). Following a rapid distribution phase, both BKM0186 and BKM0281 cleared rapidly from blood in a dose-dependent manner with BKM0281 exhibiting faster clearance compared to BKM0186. Despite fast clearance, both BKM0186 and BKM0281 showed sustained expansion of Vβ6 CD8+ T cells (as a proportion of the total CD8+ T cell compartment) (FIG. 120A and FIG. 120B) for up to 100 hours and serum levels soluble CD25 (sCD25) (FIG. 121), a serum marker of T cell activation, in monkeys administered a single IV dose of BKM0186 and BKM0281 across a dose range. In contrast, a minimal expansion of Treg was observed. The cytokine data supports dose-dependent Test Article effects on MCP-1, MIP1β, IL-10, IL-1RA, IFN-γ (FIG. 123A and FIG. 123B), IL-5, and IL-6 (FIG. 122A and FIG. 122B). The cytokine data supports an uncertain effect on IL-4 and IL-12/23 (p40). Detectible concentrations of both cytokines were present for isolated animals; however, the relationship of these changes to IL2×TCRVβ administration is uncertain due to their sporadic presence and the lack of a clear dose response. There were no Test Article-related effects on IL-8, TNF-α, IL-2, IL-13, IL-15, IL-17, IL-18, GM-CSF, or IL-1β.

### Example 54: Human Expansion Assays to Assess TriSTARs and Vb or CD3 Bispecific TCEs

Flat-bottom 96-well plates were coated overnight with 100 nM Mesothelin antigen. Thawed human PBMCs were diluted in X-Vivo15 media and incubated at 37° C., for 5 days in the presence of diluted constructs. After being stained for viability and surface antigens, cells were fixed and permeabilized using FoxP3/Transcription Factor Staining Buffer kit. Cells were stained for intracellular markers and analyzed on Cytek Aurora flow cytometer for activation (% CD25+) of CD8 T cells.

### Example 55: Human Cytotoxicity Assays to Assess TriSTARs and vb or CD3 Bispecific TCEs

Cryopreserved human PBMCs were thawed and expanded in X-Vivo15 media with 50 nM of an exemplary embodiment of the multifunctional molecule as provided herein (e.g., soluble form) for 3-4 days. PBMCs were then rested in X-Vivo15 with 100 IU/mL human IL-2 for 1-2 days. The cells of a human tumor cell line were pre-seeded overnight in 96-well, flat-bottom plates in X-Vivo15. PBMCs and constructs were then added to the pre-seeded cells of a human tumor cell line at an E:T ratio of 1:1 or 2:1 and incubated for 20 hrs. LDH assay was performed and read according to manufacturer's instructions. Briefly, supernatants were removed from cultures and diluted in LDH storage buffer, then diluted in detection reagent. After 1 hr incubation, luminescence was read on Spectramax instrument.

### Example 56: Murine Expansion Assays to Assess TriSTARs and vb or CD3 Bispecific TCEs

Flat-bottom 96-well plates were coated for 2 hours to overnight with 100 nM Mesothelin antigen in PBS. Obtained murine BALB/c spleens were smashed through 70 μm filter. RBCs were lysed and isolated PBMC (3e+5 cells in media) were incubated with diluted constructs on plate bound Mesothelin for 4 days in mouse media (RPMI+10% FBS+1.5 mM HEPES+0.5 mM BME+L-Glut+Anti-Anti+1 mM sodium pyruvate+non-essential amino acids) prior to staining for viability and surface antigens. Fixed (FoxP3/Transcription Factor Staining Buffer kit) and permeabilized cells were stained for intracellular markers and analyzed on Cytek Aurora flow cytometer for activation (% CD25+) of CD8 T cells.

### Example 57: Murine Cytotoxicity Assays to Assess TriSTARs and vb or CD3 Bispecific TCEs

Obtained murine BALB/c splenocytes were expanded in mouse media with 5-50 nM of an exemplary embodiment of the multifunctional molecule as provided herein (e.g., soluble form) for 3 days. Splenocytes were rested in mouse media with 100 IU/mL human IL-2 for 2 days. EMT-6 cells were pre-seeded overnight in 96-well flat-bottom plates in Waymouth's media+10% FBS. Media was then replaced with RPMI+1% FBS and splenocytes and constructs were added to the pre-seeded EMT-6 cells at an E:T ratio of 1:1 and incubated for 20 hours. LDH assay was performed and read according to manufacturer's instructions. Briefly, supernatants were removed from cultures and diluted in LDH storage buffer, then diluted in detection reagent. After 1 hr incubation, luminescence was read on Spectramax instrument.

### Example 58: PK Assay for BMM0449

Human Mesothelin antigen (BIM0366) was captured, and PK samples were applied. Biotinylated mouse TCRVβ antigen (BLM0404) was added as the primary detect reagent. SULFO-Streptavidin was used as the secondary detect reagent.

### Example 59: PK Assay for BNM0092

Human Mesothelin antigen (BIM0366) was captured, and PK samples containing FLAG-tagged BNM0092 were applied. Biotinylated mouse monoclonal anti-FLAG antibody was added as the primary detect reagent. SULFO-Streptavidin was used as the secondary detect reagent.

### Example 60: Exemplary Embodiments of the Multifunctional Molecule as Provided Herein Targeting CD20 as a Tumor-Associated Antigen (TAA)

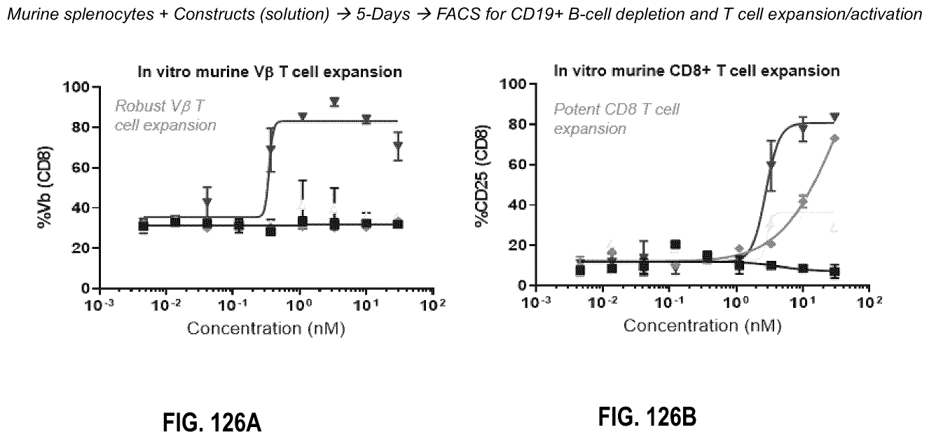

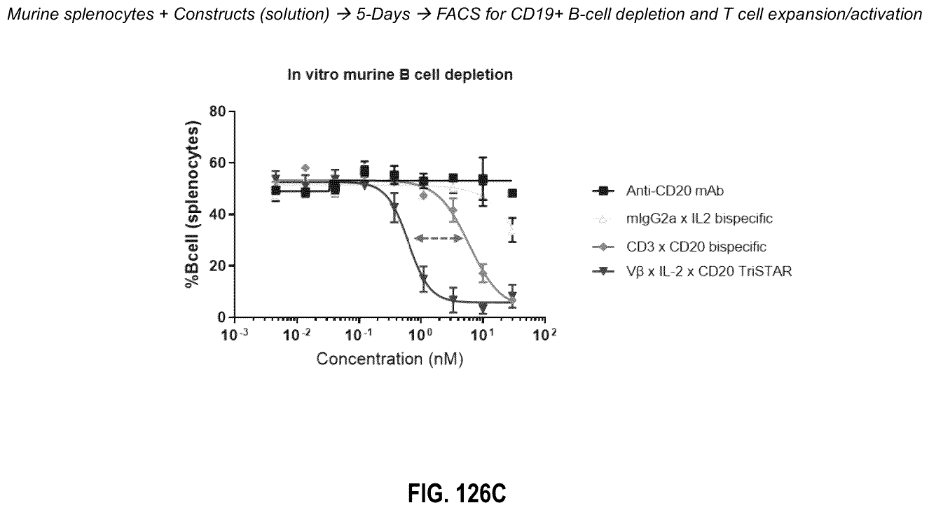

Expansion of murine CD8+& Vβ T cells along with depletion of B cells were evaluated by incubating murine splenocytes with either CD3×CD20 bispecific or Vβ×IL-2×CD20 Tristar for 5 days. Both B and T cells were assessed using FACS. Anti-TCRvb6-5, anti-CD25, anti-CD3, anti-CD8 antibodies were used to detect T cells while anti-CD19 antibody was used to detect B cells. Anti-CD20 mAb & mIgG2a×IL2 bispecific were used as controls. Robust Vβ T cell expansion was observed with the Vβ×IL-2×CD20 Tristar molecule, but not with the CD3×CD20 bispecific or control molecules (Anti-CD20 mAb & mIgG2a×IL2 bispecific). Vβ×IL-2×CD20 Tristar molecule was more potent than CD3×CD20 bispecific in expanding CD8+ T cells. In addition, the Vβ×IL-2×CD20 Tristar molecule exhibited a log higher potency than the CD3×CD20 bispecific in depleting murine B cells. (FIGS. 126A, 126B, and 126C)

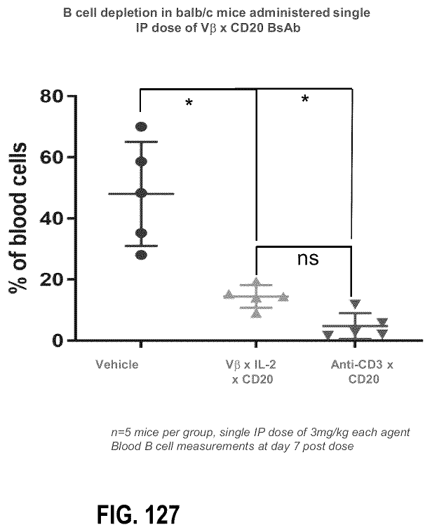

In vivo B cell depletion was evaluated by single-dose treatment of Balb/c mice with either Vβ×IL-2×CD20 Tristar molecule or with the CD3×CD20 bispecific. Test articles were administered via IP route at a dosage of 3 mg/kg and B cells were assessed using CD19 detection using FACS 7 days after the treatment. Compared to vehicle control, both constructs induced robust in vivo B cell depletion. (FIG. 127)

### Example 61: Exemplary Embodiments of the Multifunctional Molecule as Provided Herein Targeting MSLN (Mesothelin) as a Tumor-Associated Antigen (TAA)

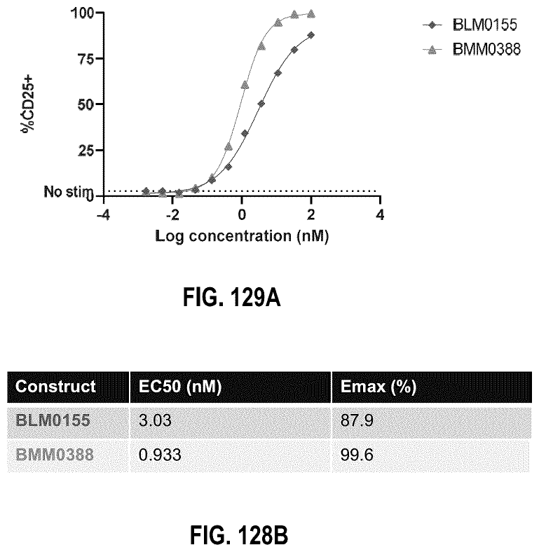

Both TriSTAR and CD3 bispecific induced robust T cell expansion in the presence of plate-bound Mesothelin, but CD3 bispecific treatment resulted in slightly more expansion. (FIGS. 128A and 128B)

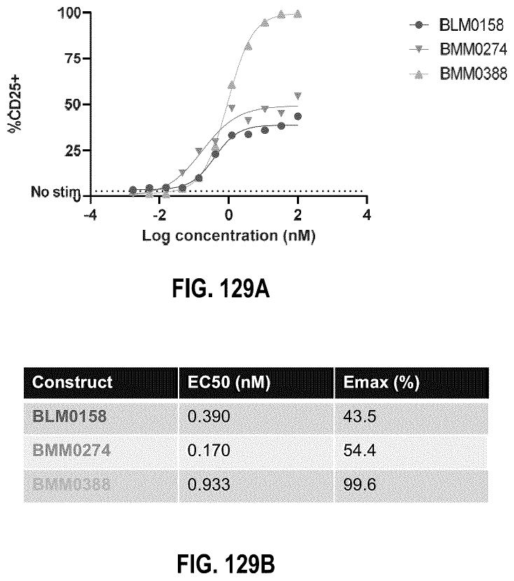

Both Vβ (an exemplary embodiment of the multifunctional molecule as provided herein) and CD3 bispecifics expanded T cells in the presence of plate-bound Mesothelin, but expansion was significantly higher with CD3 bispecific. (FIGS. 129A and 129B)

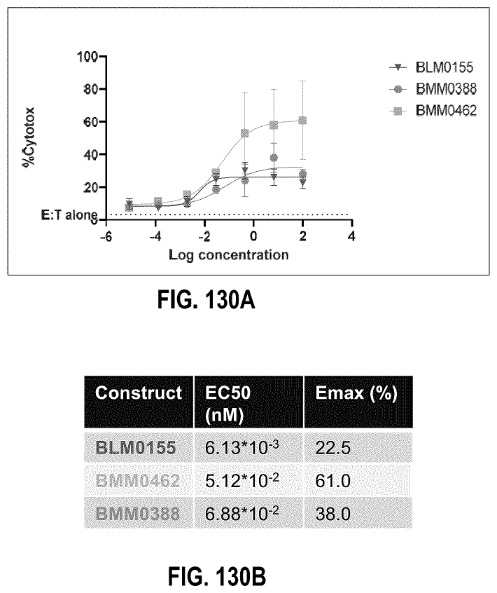

Both Tristar (Vβ×IL-2×MSLN) constructs, which are exemplary embodiments of the multifunctional molecule as provided herein, and CD3×MSLN bispecific construct induced cytotoxicity vs a human tumor cell line. When the tumor engaging arm is bivalent (BMM0462), higher degree of tumor cell-killing was observed. (FIGS. 130A and 130B)

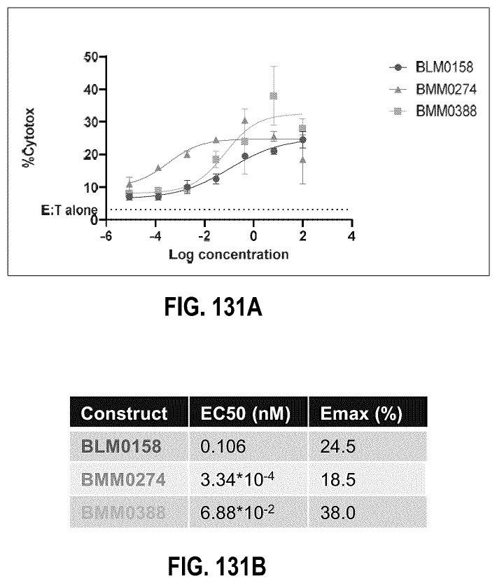

Vβ×MSLN (1×1 or 2×2) bispecifics, which are exemplary embodiments of the multifunctional molecule as provided herein, and CD3×MSLN bispecifics were tested for their ability to induce cytotoxicity vs human tumor cell line. While all tested bispecifics induced similar levels of maximum % cytotoxicity (Emax 18-38%), the EC50 for Vβ×MSLN (2×2) was significantly lower than EC50 for both Vβ×MSLN (1 ×1) and CD3×MSLN bispecifics. (FIGS. 131A and 131B)

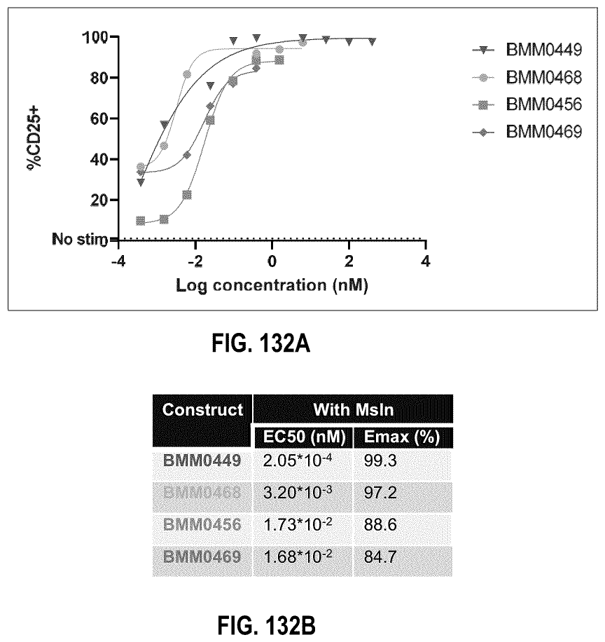

Although robust expansion of murine T cells was observed with Tristar (Vβ×IL-2×MSLN) constructs, which are exemplary embodiments of the multifunctional molecule as provided herein, and CD3×MSLN bispecific constructs, Tristar molecules were more potent Valency of the TAA arm (BMM0449 vs BMM0468 or BMM0456 vs BMM0469) did not have a significant impact on potency. (FIGS. 132A and 132B)

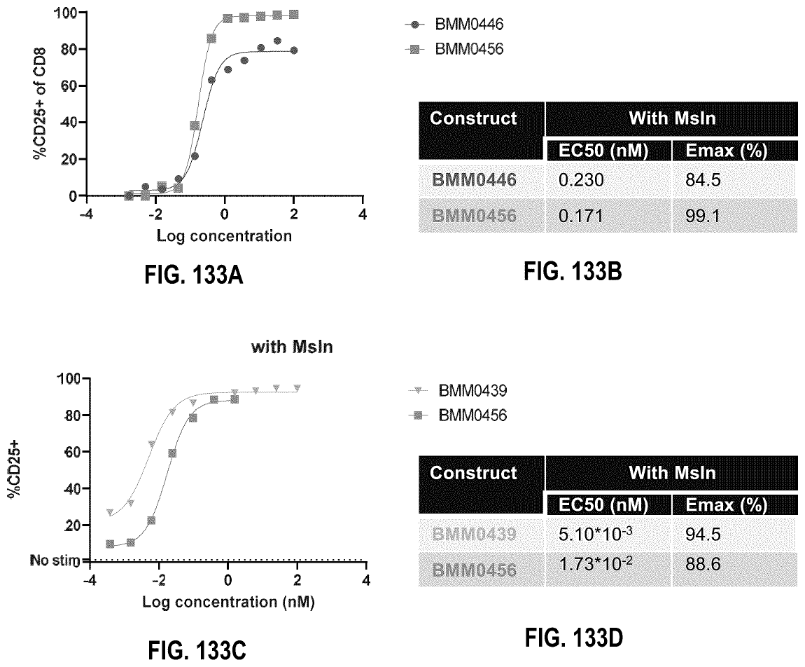

Across several independent experiments, the ability of Vβ×MSLN bispecifics, which are exemplary embodiments of the multifunctional molecule as provided herein, and CD3×MSLN bispecifics to induce murine CD& T cell expansion was compared. In the presence of plate-bound Mesothelin, expansion induced by 1×1 Vβ×MSLN (BMM0446) & 1×1 CD3×MSLN (BMM0456) were comparable. In a separate experiment (EXP23005483), while robust expansion was observed in the presence of MSLN with 2×2 Vβ×MSLN (BMM0439). 1×1 CD3×MSLN (BMM0456), or 2×1 CD3×MSLN (BMM0469), in the absence of MSLN antigen, there was either no expansion (BMM0439) or it was significantly lower (BMM0456 & BMM0469). In a third separate experiment (EXP23005549), the presence of MSLN significantly increased the potency of expansion for 2×2 Vβ×MSLN (BMM0439), 2×1 Vβ×MSLN (BNM0096), or 2×1 CD3×MSLN (BMM0469). (FIGS. 133A-133D)

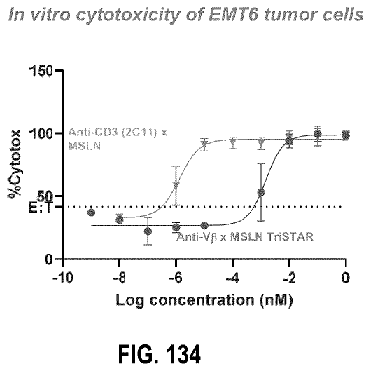

In the presence of plate-bound Mesothelin antigen, both Vβ×IL2×MSLN Tristar, which is an exemplary embodiment of the multifunctional molecule as provided herein, and CD3×MSLN both expanded murine T cells, but CD3 bispecific was more potent. Similarly. CD3×MSLN bispecific was more potent than the Vβ×IL2×MSLN Tristar, which is an exemplary embodiment of the multifunctional molecule as provided herein, in inducing cytotoxicity vs EMT6 tumor cells. (FIG. 134)

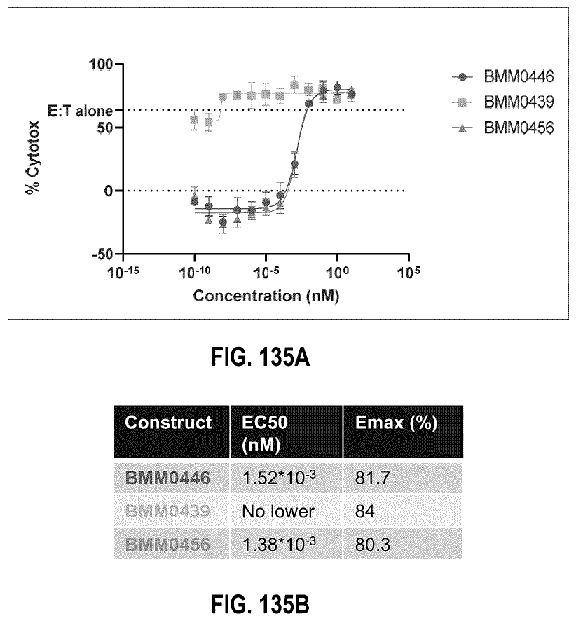

Across several independent experiments, the ability of Vβ×MSLN bispecifics, which are exemplary embodiments of the multifunctional molecule as provided herein, and CD3×MSLN bispecifics to induce cytotoxicity vs a murine tumor cell line was evaluated. In one experiment (EXP2300014), 2×2 Vβ×MSLN (BMM0439), 1×1 Vβ-MSLN (BMM0446), and 1×1 CD3-MSLN (BMM0456) bispecifics were compared. While all three induced potent cytoxicity. 2×2 Vβ-MSLN (BMM0439) was significantly more potent than 1×1 Vβ×MMSLN (BMM0446), and 1×1 CD3×MSLN (BMM0456). Similar results were obtained in a separate assay (EXP23005472) where 2×2 Vβ×MSLN (BMM0439) was shown to be more potent than both 1×1 CD3×MSLN (BMM0456) and 2×1 CD3×MSLN (BMM0469). In two separate assays (EXP23005466 & EXP23005466), increasing the valency of TAA engaging arm (monovalent BMM0456 vs bivalent BMM0469) did not significantly increase potency. In a separate assay (EXP23005562), T cell cytotoxicity was evaluated for 2×2 Vβ×MSLN (BMM0439), 2×1 Vβ×MSLN (BNM0096) and 2×1 CD3×MSLN (BMM0469) constructs. BMM0439 was more potent than BMM0469 & BNM0096. (FIGS. 135A and 135B)

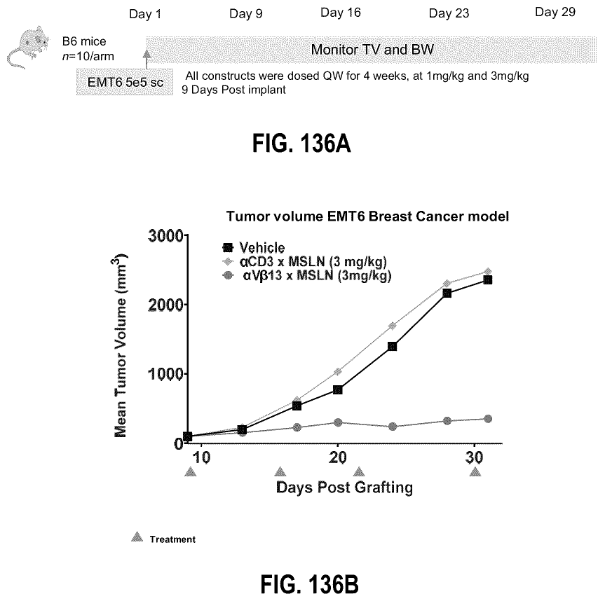

While CD3×MSLN showed no in vivo anti-tumor activity in the EMT6 model, Vβ×IL2×MSLN Tristar, which is an exemplary embodiment of the multifunctional molecule as provided herein, showed significant anti-tumor activity. (FIGS. 136A and 136B)

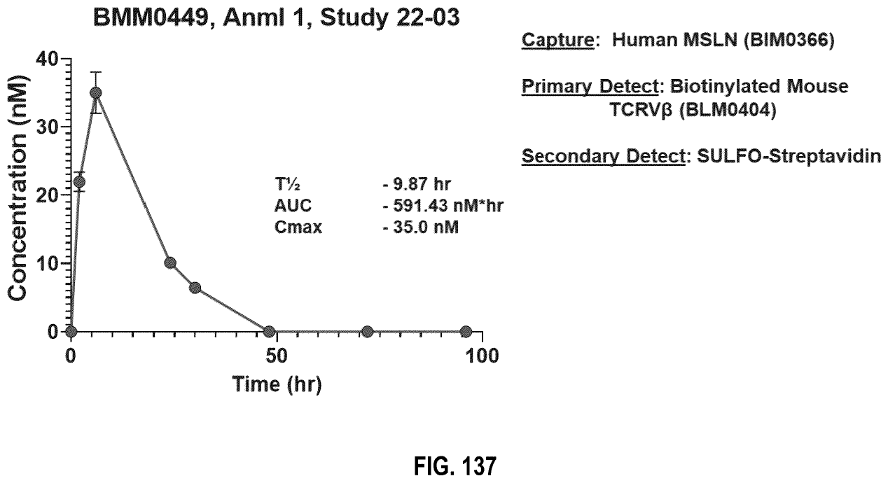

Pharmacokinetics of Vβ×IL2×MSLN Tristar (BMM0449), which is an exemplary embodiment of the multifunctional molecule as provided herein, was evaluated Balb/c mice. Half-life for clearance of the Tristar was measured as 9.9 hours. (FIG. 137)

### Example 62: Exemplary Embodiments of the Multifunctional Molecule as Provided Herein Targeting Gp75 (Tryp1) as a Tumor-Associated Antigen (TAA)

C57 mice were inoculated with 2e5 B16-F19 cells per mouse. When the mice reached an average of 50 mm³ the mice were then randomized 10 mice per group and dosed with BNM0104 and BNM0869 at 5 mg/kg BIW for 4 weeks along with PBS for the vehicle. Tumor measurements and body weights were collected twice a week.

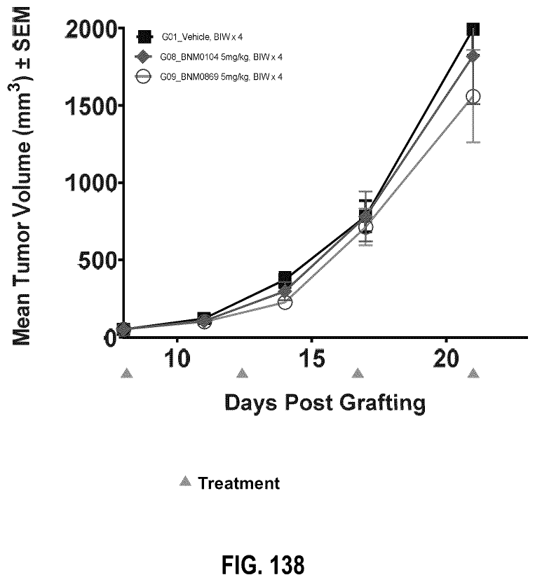

1×1 and 2×2 Vβ×gp75 TCEs, which are exemplary embodiments of the multifunctional molecule as provided herein, were tested in vivo in B16 solid tumor model. The anti-tumor activity was not significantly different than the vehicle control. (FIG. 138)

C57 mice were inoculated with 2e5 B16-F19 cells per mouse. When the mice reached an average of 50 mm³ the mice were then randomized 10 mice per group. Mice were then dosed with BNM0104, BNM0869 at 5 mg/kg, and BKM0307 at 1 mg/kg BIW for 4 weeks along with PBS for the vehicle. The combination groups were dosed with BKM0307 on day 0, 10, 20 and BNM0104 or BNM0869 were dosed day 3, 7, 13, 17. Tumor measurements and body weights were collected twice a week.

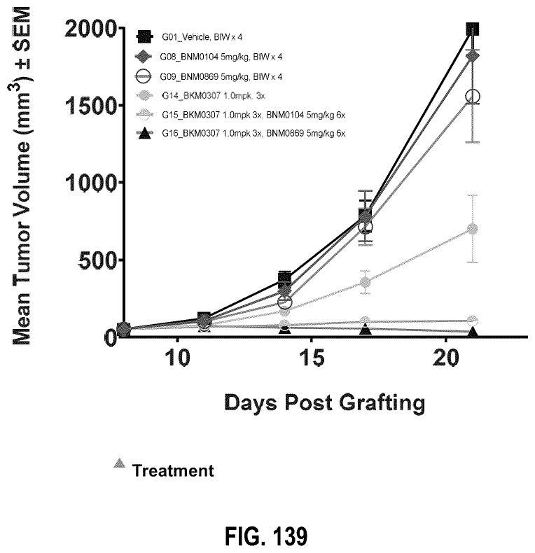

While 1×1 and 2×2 Vβ×gp75 TCEs, which are exemplary embodiments of the multifunctional molecule as provided herein, in vivo did not show significant anti-tumor activity as single-agents, they showed significant anti-tumor activity when combined with an exemplary multifunctional molecule comprising a Vβ6 binder and IL-2 (BKM0307). Efficacy with the combination (Groups 15 & 16) was higher than all single-agents including an exemplary multifunctional molecule comprising a Vβ6 binder and IL-2 (Group 14). 1×1 and 2×2 Vβ×gp75 T cell engagers are active in B16F10 tumor model when dosed with or following an exemplary multifunctional molecule comprising a Vβ6 binder and IL-2. (FIG. 139)

C57 mice were inoculated with 2e5 B16-F19 cells per mouse. When the mice reached an average of 50 mm³ the mice were then randomized 10 mice per group then dosed with BNM0094, which is an exemplary embodiment of the multifunctional molecule as provided herein, at 1 and 03 mg/kg and BNM0092 at 2 mg/kg BIW for 4 weeks along with PBS for the vehicle. Tumor measurements and body weights were collected twice a week.

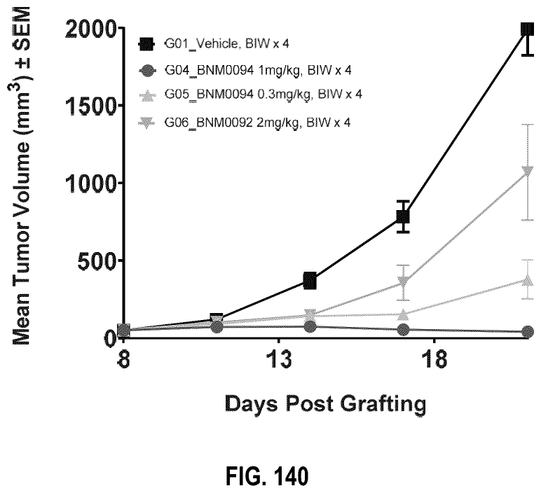

In the refractory B16F10 model, Vβ×IL2×gp75 Tristar (BNM0094), which is an exemplary embodiment of the multifunctional molecule as provided herein, showed significant anti-tumor activity, whereas CD3×gp75 bispecific (BNM0092) showed limited in vivo anti-tumor activity. Significant anti-tumor activity in refractory B16 mouse with TriSTAR TCE was observed. (FIG. 140)

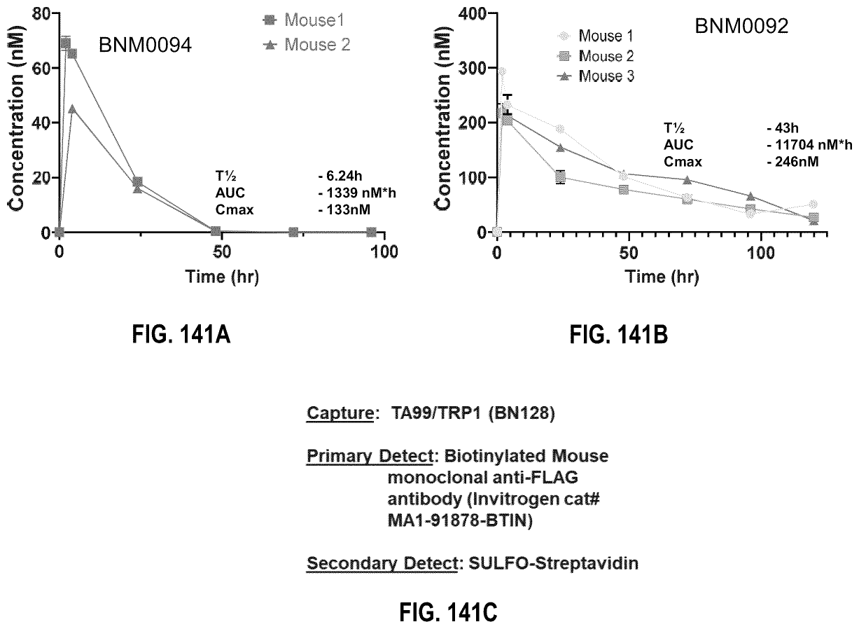

Mice were inoculated with 2e5 B16-F19 cells per mouse. When the mice reached an average of 100 mm³ the mice were then randomized 3 mice per group. Mice were injected with BNM0094, which is an exemplary embodiment of the multifunctional molecule as provided herein, at 1 mg/kg or BNM0092 at 2 mg/kg. Day 0 prebleed, 2 hr. 4 hr, 24 hr, 48 hr. 72 hr, 96 hr, and 120 hr serum samples were collected. Pharmacokinetics of Vβ×IL2×gp75 Tristar (BNM0094), which is an exemplary embodiment of the multifunctional molecule as provided herein, and CD3×gp75 bispecific (BNM0092) were evaluated in the B16F10 mice. Half-lives for the Tristar and the CD3-bispecific were 6.2 & 43 hours respectively. (FIGS. 141A-141C)

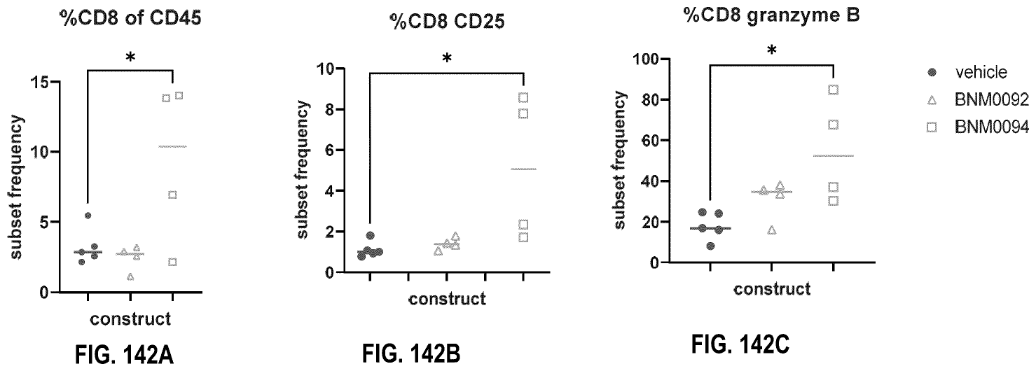

Pharmacodynamic properties of Vβ×IL2×gp75 Tristar (BNM0094), which is an exemplary embodiment of the multifunctional molecule as provided herein, and CD3×gp75 bispecific (BNM0092) were assessed in B16F10 mice. Compared to treatment with the CD3×gp75 bispecific (BNM0092), treatment with Vβ×IL2×gp75 Tristar (BNM0094) resulted in a higher levels of CD8+ T cell expansion in the tumor. (FIGS. 142A-142C)

Mice were inoculated with 2e5 B16-F19 cells per mouse. When the mice reached an average of 100 mm³ the mice were then randomized 5 mice per group. Mice were injected with BNM0094, which is an exemplary embodiment of the multifunctional molecule as provided herein, at 1 mg/kg or BNM0092 at 2 mg/kg BIW, 3 doses. Tumors and whole blood were then collected 3 days after the final dose.

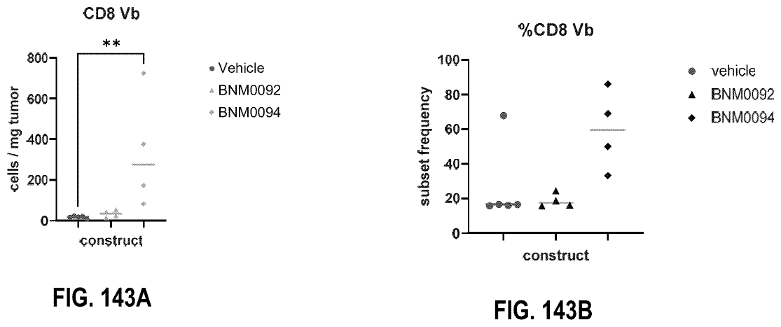

Compared to treatment with the CD3×gp75 bispecific (BNM0092), treatment with Vβ×IL2×gp75 Tristar (BNM0094) resulted in a higher levels of Vβ13+ T cell expansion in the tumor. (FIGS. 143A and 143B)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Those skilled in the art will recognize, or be able to ascertain using no more than routine experimentation, many equivalents to the specific embodiments of the invention described herein. Such equivalents are intended to be encompassed by the following claims.

TABLE 21
Exemplary Construct Sequences

| | | | |
|---|---|---|---|
| | SEQ | | |
| | ID | | |
| Chain | NO: | Component | Sequence |

TCRVβ/IL15-sushi bispecific

| | | | |
|---|---|---|---|
| Chain 1: | 3517 | Full Sequence | QVQLVQSGAEVKKPGSSVKVSCKASGHDFRLTYIHWVRQAPGQGLE |
| TCRVβ- | | | WMGRVSAGSGNVKYNEKFKGRVTITADTSTSTAYMELSSLRSEDTA |
| Fc_Hole_Cys_ | | | VYYCAVSYYSYDVLDYWGQGTTVTVSSASTKGPSVFPLAPSSKSTS |
| N297A heavy | | | GGTAALGCLVKDYFPEPVTVSWNSGALTSGVHTFPAVLQSSGLYSLS |
| chain (CDRs | | | SVVTVPSSSLGTQTYICNVNHKPSNTKVDKRVEPKSCDKTHTCPPCP |
| underlined) | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2: | 3519 | Full Sequence | ITCPPPMSVEHADIWVKSYSLYSRERYICNSGFKRKAGTSSLTECVLN |
| IL15RA_Sushi- | | | KATNVAHWTTPSLKCIRDPALVHQRPAPPSGGSGGGGSGGGSGGGG |
| GS linker- | | | SGGNWVNVISDLKKIEDLIQSMHIDATLYTESDVHPSCKVTAMKCFL |
| IL15-GS | | | LELQVISLESGDASIHDTVENLIILANNSLSSNGNVTESGCKECEELEE |
| linker- | | | KNIKEFLQSFVHIVQMFINTSGGGGSGGGGSDKTHTCPPCPAPELLGG |
| Fc_Knob_Cys_ | | | PSVFLFPPKPKDTLMISRTPEVTCVVVDVSHEDPEVKENWYVDGVEV |
| N297A | | | HNAKTKPREEQYASTYRVVSVLTVLHQDWLNGKEYKCKVSNKALP |
| | | | APIEKTISKAKGQPREPQVYTLPPCREEMTKNQVSLWCLVKGFYPSDI |
| | | | AVEWESNGQPENNYKTTPPVLDSDGSFFLYSKLTVDKSRWQQGNVF |
| | | | SCSVMHEALHNHYTQKSLSLSPGK |
| | 3523 | IL15RA Sushi | ITCPPPMSVEHADIWVKSYSLYSRERYICNSGFKRKAGTSSLTECVLN |
| | | | KATNVAHWTTPSLKCIRDPALVHQRPAPP |
| | 3524 | linker | SGGSGGGGSGGGSGGGGSGG |
| | 2170 | human IL-15 | NWVNVISDLKKIEDLIQSMHIDATLYTESDVHPSCKVTAMKCFLLEL |
| | | | QVISLESGDASIHDTVENLIILANNSLSSNGNVTESGCKECEELEEKNI |
| | | | KEFLQSFVHIVQMFINTS |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | 3518 | Full Sequence | DIQMTQSPSFLSASVGDRVTITCKASQNVADRVVWHQQKPGKAPKA |
| TCRVβ light | | | LIYSSSHRYKGVPSRFSGSGSGTEFTLTISSLQPEDFATYFCQQFKSYP |
| chain (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLILSKADYEKHKVYACEVTHQGL |
| | | region | SSPVTKSFNRGEC |

TCRVβ/IL15 bispecific

| | | | |
|---|---|---|---|
| Chain 1: | 3517 | Full Sequence | QVQLVQSGAEVKKPGSSVKVSCKASGHDFRLTYIHWVRQAPGQGLE |
| TCRVβ- | | | WMGRVSAGSGNVKYNEKFKGRVTITADTSTSTAYMELSSLRSEDTA |
| Fc_Hole_Cys_ | | | VYYCAVSYYSYDVLDYWGQGTTVTVSSASTKGPSVFPLAPSSKSTS |
| N297A heavy | | | GGTAALGCLVKDYFPEPVTVSWNSGALTSGVHTFPAVLQSSGLYSLS |
| chain (CDRs | | | SVVTVPSSSLGTQTYICNVNHKPSNTKVDKRVEPKSCDKTHTCPPCP |
| underlined) | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2: | 3520 | Full Sequence | NWVNVISDLKKIEDLIQSMHIDATLYTESDVHPSCKVTAMKCFLLEL |
| IL15-GS | | | QVISLESGDASIHDTVENLIILANNSLSSNGNVTESGCKECEELEEKNI |
| linker- | | | KEFLQSFVHIVQMFINTSGGGGSGGGGSDKTHTCPPCPAPELLGGPSV |
| Fc_Knob_Cys_ | | | FLFPPKPKDTLMISRTPEVTCVVVDVSHEDPEVKFNWYVDGVEVHN |
| N297A | | | AKTKPREEQYASTYRVVSVLTVLHQDWLNGKEYKCKVSNKALPAPI |
| | | | EKTISKAKGQPREPQVYTLPPCREEMTKNQVSLWCLVKGFYPSDIAV |
| | | | EWESNGQPENNYKTTPPVLDSDGSFFLYSKLTVDKSRWQQGNVFSC |
| | | | SVMHEALHNHYTQKSLSLSPGK |
| | 2170 | human IL-15 | NWVNVISDLKKIEDLIQSMHIDATLYTESDVHPSCKVTAMKCFLLEL |
| | | | QVISLESGDASIHDTVENLIILANNSLSSNGNVTESGCKECEELEEKNI |
| | | | KEFLQSFVHIVQMFINTS |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | 3518 | Full Sequence | DIQMTQSPSFLSASVQDRVTITCKASQNVADRVVWHQQKPGKAPKA |
| TCRVβ light | | | LIYSSSHRYKGVPSRFSGSGSGTEFTLTISSLQPEDFATYFCQQFKSYP |
| chain (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

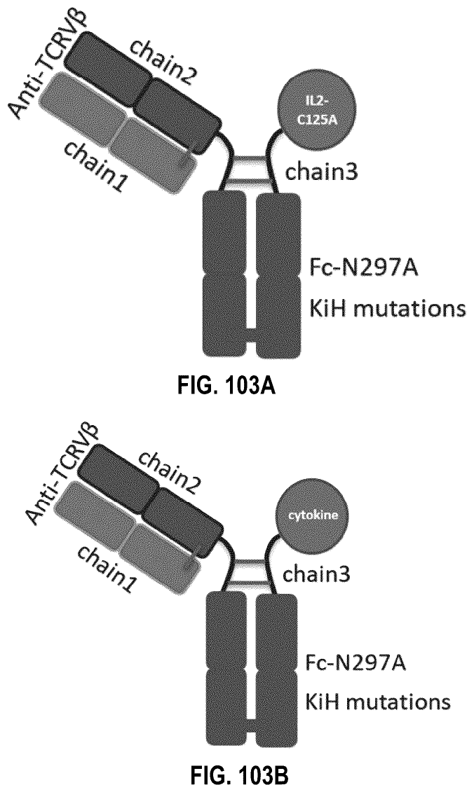

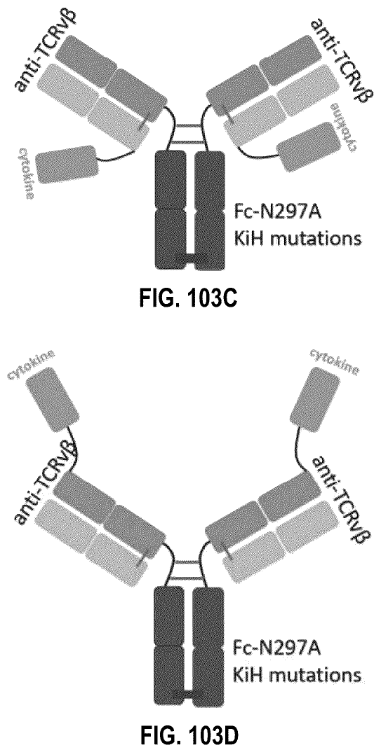

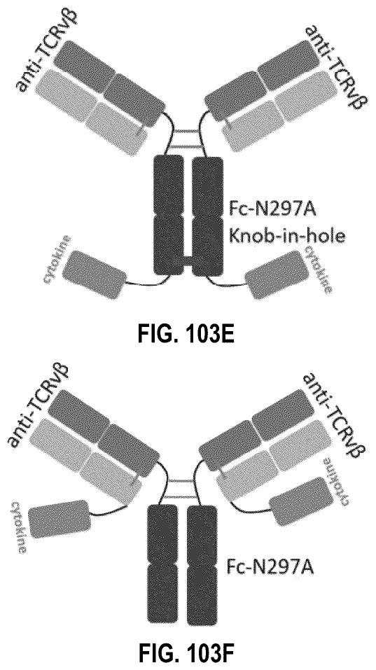

BKM0186 (FIG. 103A)

| | | | |
|---|---|---|---|
| Chain 1 | 3517 | Full Sequence | QVQLVQSGAEVKKPGSSVKVSCKASGHDFRLTYIHWVRQAPGQGLE |
| (heavy chain) | | | WMGRVSAGSGNVKYNEKFKGRVTITADTSTSTAYMELSSLRSEDTA |
| (CDRs | | | VYYCAVSYYSYDVLDYWGQGTTVTVSSASTKGPSVFPLAPSSKSTS |
| underlined) | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2 | 3521 | Full Sequence | APTSSSTKKTQLQLEHLLLDLQMILNGINNYKNPKLTRMLTFKFYMP |
| (IL2-Fc) | | | KKATELKHLQCLEEELKPLEEVLNLAQSKNFHLRPRDLISNINVIVLE |
| | | | LKGSETTFMCEYADETATIVEFLNRWITFAQSIISTLTGGGGSGGGGS |
| | | | DKTHTCPPCPAPELLGGPSVFLFPPKPKDTLMISRTPEVTCVVVDVSH |
| | | | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| | 2270 | IL-2 C125A | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLT |
| | 3308 | linker | GGGGGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3 | 3518 | Full Sequence | DIQMTQSPSFLSASVGDRVTITCKASQNVADRVVWHQQKPGKAPKA |
| (light chain) | | | LIYSSSHRYKGVPSRFSGSGSGTEFTLTISSLQPEDFATYFCQQFKSYP |
| (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

mBKM0186

| | | | |
|---|---|---|---|
| Chain 1 | 3529 | Full Sequence | QVQLQQSGTELMKPGASVKISCKASGYTFSNYWIEWIKQRPGHGLE |
| (heavy chain) | | | WVGEILPGAGPTNYNEKFKGKATFTADSSSNTAYMQLSSLTSEDSAV |
| (CDRs | | | YYCARTDYDYDWFAYWGQGTLVTVSAAKTTAPSVYPLAPVSGDTT |
| underlined) | | | GSSVTLGCLVKGYFPEPVTLTWNSGSLSSGVHTFPAVLQSDLYTLSSS |
| | | | VTVTSSTWPSQSITCNVAHPASSTKVDKKIEPKSCDKTHTCPPCPAPN |
| | | | AAGGPSVFIFPPKIKDVLMISLSPIVTCVVVDVSEDDPDVQISWFVNN |
| | | | VEVHTAQTQTHREDYNSTLRVVSALPIQHQDWMSGKEFKCKVNNK |
| | | | DLGAPIERTISKPKGSVRAPQVCVLPPPEEEMTKKQVTLSCAVTDFMP |
| | | | EDIYVEWTNNGKTELNYKNTEPVLDSDGSYFMVSKLRVEKKNWVE |
| | | | RNSYSCSVVHEGLHNHHTTKSFSRTPGKEPEA |
| | 3548 | Full Sequence | QVQLQQSGTELMKPGASVKISCKASGYTFSNYWIEWIKQRPGHGLE |
| | | without tag | WVGEILPGAGPTNYNEKFKGKATFTADSSSNTAYMQLSSLTSEDSAV |
| | | | YYCARTDYDYDWFAYWGQGTLVTVSAAKTTAPSVYPLAPVSGDTT |
| | | | GSSVTLGCLVKGYFPEPVTLTWNSGSLSSGVHTFPAVLQSDLYTLSSS |
| | | | VTVTSSTWPSQSITCNVAHPASSTKVDKKIEPKSCDKTHTCPPCPAPN |
| | | | AAGGPSVFIFPPKIKDVLMISLSPIVTCVVVDVSEDDPDVQISWFVNN |
| | | | VEVHTAQTQTHREDYNSTLRVVSALPIQHQDWMSGKEFKCKVNNK |
| | | | DLGAPIERTISKPKGSVRAPQVCVLPPPEEEMTKKQVTLSCAVTDFMP |
| | | | EDIYVEWTNNGKTELNYKNTEPVLDSDGSYFMVSKLRVEKKNWVE |
| | | | RNSYSCSVVHEGLHNHHTTKSFSRTPGK |
| | 3530 | anti-TCRvβ VH | QVQLQQSGTELMKPGASVKISCKASGYTFSNYWIEWIKQRPGHGLE |
| | | from murine | WVGEILPGAGPTNYNEKFKGKATFTADSSSNTAYMQLSSLTSEDSAV |
| | | MR5-2 | YYCARTDYDYDWFAYWGQGTLVTVSAAKTTAPSVYPLAPVSGDTT |
| | | | GSSVTLGCLVKGYFPEPVTLTWNSGSLSSGVHTFPAVLQSDLYTLSSS |
| | | | VTVTSS |
| | 3531 | mouse Fc heavy | TWPSQSITCNVAHPASSTKVDKKIEPKSCDKTHTCPPCPAPNAAGGPS |
| | | chain comprising | VFIFPPKIKDVLMISLSPIVTCVVVDVSEDDPDVQISWFVNNVEVHTA |
| | | LALAPG | QTQTHREDYNSTLRVVSALPIQHQDWMSGKEFKCKVNNKDLGAPIE |
| | | mutations | RTISKPKGSVRAPQVCVLPPPEEEMTKKQVTLSCAVTDFMPEDIYVE |
| | | (L234A L235A | WTNNGKTELNYKNTEPVLDSDGSYFMVSKLRVEKKNWVERNSYSC |
| | | and P329G) | SVVHEGLHNHHTTKSFSRTPGK |
| | 3547 | Tag | EPEA |
| Chain 2 | 3532 | Full Sequence | APTSSSTKKTQLQLEHLLLDLQMILNGINNYKNPKLTRMLTFKFYMP |
| (IL2-Fc) | | | KKATELKHLQCLEEELKPLEEVLNLAQSKNFHLRPRDLISNINVIVLE |
| | | | LKGSETTFMCEYADETATIVEFLNRWITFCQSIISTLTGGGGSGGGGS |
| | | | DKTHTCPPCPAPNAAGGPSVFIFPPKIKDVLMISLSPIVTCVVVDVSED |
| | | | DPDVQISWFVNNVEVHTAQTQTHREDYNSTLRVVSALPIQHQDWMS |
| | | | GKEFKCKVNNKDLGAPIERTISKPKGSVRAPQVYVLPPCEEEMTKKQ |
| | | | VTLWCMVTDFMPEDIYVEWTNNGKTELNYKNTEPVLDSDGSYFMY |
| | | | SKLRVEKKNWVERNSYSCSVVHEGLHNHHTTKSFSRTPGKGGGGSH |
| | | | HHHHHHH |
| | 3549 | Full Sequence | APTSSSTKKTQLQLEHLLLDLQMILNGINNYKNPKLTRMLTFKFYMP |
| | | without tag | KKATELKHLQCLEEELKPLEEVLNLAQSKNFHLRPRDLISNINVIVLE |
| | | | LKGSETTFMCEYADETATIVEFLNRWITFCQSIISTLTGGGGSGGGGS |
| | | | DKTHTCPPCPAPNAAGGPSVFIFPPKIKDVLMISLSPIVTCVVVDVSED |
| | | | DPDVQISWFVNNVEVHTAQTQTHREDYNSTLRVVSALPIQHQDWMS |
| | | | GKEFKCKVNNKDLGAPIERTISKPKGSVRAPQVYVLPPCEEEMTKKQ |
| | | | VTLWCMVTDFMPEDIYVEWTNNGKTELNYKNTEPVLDSDGSYFMY |
| | | | SKLRVEKKNWVERNSYSCSVVHEGLHNHHTTKSFSRTPGK |
| | 2191 | IL-2 | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFCQSIISTLT |
| | 3308 | linker | GGGGSGGGGS |
| | 3533 | mouse Fc heavy | DKTHTCPPCPAPNAAGGPSVFIFPPKIKDVLMISLSPIVTCVVVDVSED |
| | | chain comprising | DPDVQISWFVNNVEVHTAQTQTHREDYNSTLRVVSALPIQHQDWMS |
| | | LALAPG | GKEFKCKVNNKDLGAPIERTISKPKGSVRAPQVYVLPPCEEEMTKKQ |
| | | mutations | VTLWCMVTDFMPEDIYVEWTNNGKTELNYKNTEPVLDSDGSYFMY |
| | | (L234A L235A | SKLRVEKKNWVERNSYSCSVVHEGLHNHHTTKSFSRTPGK |
| | | and P329G) | |
| | 3534 | Tag | GGGGSHHHHHHHH |
| Chain 3 | 3526 | Full Sequence | DIVMSQSPSSLAVSVGEKVTMSCKSSQSLLYSGNQKNYLAWYQQKP |
| (light chain) | | | GQSPKLLIYWASTRESGVPDRFTGSGSGTDFTLTINSVKAEDLTVYYC |
| (CDRs | | | QQYYGYPRTFGGGTKVEIKRADAAPTVSIFPPSSEQLTSGGASVVCFL |
| underlined) | | | NNFYPKDINVKWKIDGSERQNGVLNSWTDQDSKDSTYSMSSTLTLT |
| | | | KDEYERHNSYTCEATHKTSTSPIVKSFNRNEC |
| | 3527 | anti-TCRvβ VL | DIVMSQSPSSLAVSVGEKVTMSCKSSQSLLYSGNQKNYLAWYQQKP |
| | | from murine | GQSPKLLIYWASTRESGVPDRFTGSGSGTDFTLTINSVKAEDLTVYYC |
| | | MR5-2 | QQYYGYPRTFGGGTKVEIK |
| | 3528 | Immunoglobulin | RADAAPTVSIFPPSSEQLTSGGASVVCFLNNFYPKDINVKWKIDGSER |
| | | constant region | QNGVLNSWTDQDSKDSTYSMSSTLTLTKDEYERHNSYTCEATHKTS |
| | | | TSPIVKSFNRNEC |

mBKM0281

| | | | |
|---|---|---|---|
| Chain 1 | 3536 | Full Sequence | QVQLQQSGTELMKPGASVKISCKASGYTFSNYWIEWIKQRPGHGLE |
| (heavy chain) | | | WVGEILPGAGPTNYNEKFKGKATFTADSSSNTAYMQLSSLTSEDSAV |
| (CDRs | | | YYCARTDYDYDWFAYWGQGTLVTVSAAKTTAPSVYPLAPVSGDTT |
| underlined) | | | GSSVTLGCLVKGYFPEPVTLTWNSGSLSSGVHTFPAVLQSDLYTLSSS |
| | | | VTVTSSTWPSQSITCNVAHPASSTKVDKKIEPKSCDKTHTCPPCPAPN |
| | | | AAGGPSVFIFPPKIKDVLMISLSPIVTCVVVDVSEDDPDVQISWFVNN |
| | | | VEVHTAQTQTHREDYNSTLRVVSALPIQHQDWMSGKEFKCKVNNK |
| | | | DLGAPIERTISKPKGSVRAPQVYVLPPPEEEMTKKQVTLTCMVTDFM |
| | | | PEDIYVEWTNNGKTELNYKNTEPVLDSDGSYFMYSKLRVEKKNWV |
| | | | ERNSYSCSVVHEGLHNHHTTKSFSRTPGKEPEA |
| | 3530 | anti-TCRvβ VH | QVQLQQSGTELMKPGASVKISCKASGYTFSNYWIEWIKQRPGHGLE |
| | | from murine | WVGEILPGAGPTNYNEKFKGKATFTADSSSNTAYMQLSSLTSEDSAV |
| | | MR5-2 | YYCARTDYDYDWFAYWGQGTLVTVSAAKTTAPSVYPLAPVSGDTT |
| | | | GSSVTLGCLVKGYFPEPVTLTWNSGSLSSGVHTFPAVLQSDLYTLSSS |
| | | | VTVTSS |
| | 3537 | mouse Fc heavy | TWPSQSITCNVAHPASSTKVDKKIEPKSCDKTHTCPPCPAPNAAGGPS |
| | | chain comprising | VFIFPPKIKDVLMISLSPIVTCVVVDVSEDDPDVQISWFVNNVEVHTA |
| | | LALAPG | QTQTHREDYNSTLRVVSALPIQHQDWMSGKEFKCKVNNKDLGAPIE |
| | | mutations | RTISKPKGSVRAPQVYVLPPPEEEMTKKQVTLTCMVTDFMPEDIYVE |
| | | (L234A L235A | WTNNGKTELNYKNTEPVLDSDGSYFMYSKLRVEKKNWVERNSYSC |
| | | and P329G) | SVVHEGLHNHHTTKSFSRTPGKEPEA |
| Chain 2 | 3535 | Full Sequence | DIVMSQSPSSLAVSVGEKVTMSCKSSQSLLYSGNQKNYLAWYQQKP |
| (light chain- | | | GQSPKLLIYWASTRESGVPDRFTGSGSGTDFTLTINSVKAEDLTVYYC |
| IL2) | | | QQYYGYPRTFGGGTKVEIKRADAAPTVSIFPPSSEQLTSGGASVVCFL |
| (CDRs | | | NNFYPKDINVKWKIDGSERQNGVLNSWTDQDSKDSTYSMSSTLTLT |
| underlined) | | | KDEYERHNSYTCEATHKTSTSPIVKSFNRNECGGGGSGGGGSGGGGS |
| | | | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFCQSIISTLT |
| | 3527 | anti-TCRvβ VL | DIVMSQSPSSLAVSVGEKVTMSCKSSQSLLYSGNQKNYLAWYQQKP |
| | | from murine | GQSPKLLIYWASTRESGVPDRFTGSGSGTDFTLTINSVKAEDLTVYYC |
| | | MR5-2 | QQYYGYPRTFGGGTKVEIK |
| | 3528 | Immunoglobulin | RADAAPTVSIFPPSSEQLTSGGASVVCFLNNFYPKDINVKWKIDGSER |
| | | constant region | QNGVLNSWTDQDSKDSTYSMSSTLTLTKDEYERHNSYTCEATHKTS |
| | | | TSPIVKSFNRNEC |
| | 3309 | linker | GGGGSGGGGSGGGGS |
| | 2191 | IL-2 | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFCQSIISTLT |

BMM0317(IL-7)

| | | | |
|---|---|---|---|
| Chain 1: | 3517 | Full Sequence | QVQLVQSGAEVKKPGSSVKVSCKASGHDFRLTYIHWVRQAPGQGLE |
| TCRVβ- | | | WMGRVSAGSGNVKYNEKFKGRVTITADTSTSTAYMELSSLRSEDTA |
| Fc_Hole_Cys_ | | | VYYCAVSYYSYDVLDYWGQGTTVTVSSASTKGPSVFPLAPSSKSTS |
| N297A heavy | | | GGTAALGCLVKDYFPEPVTVSWNSGALTSGVHTFPAVLQSSGLYSLS |
| chain (CDRs | | | SVVTVPSSSLGTQTYICNVNHKPSNTKVDKRVEPKSCDKTHTCPPCP |
| underlined) | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2: | 3539 | Full Sequence | DCDIEGKDGKQYESVLMVSIDQLLDSMKEIGSNCLNNEFNFFKRHIC |
| IL-7-GS | | | DANKEGMFLFRAARKLRQFLKMNSTGDFDLHLLKVSEGTTILLNCT |
| linker-IL15- | | | GQVKGRKPAALGEAQPTKSLEENKSLKEQKKLNDLCFLKRLLQEIKT |
| GS linker- | | | CWNKILMGTKEHGGGGSGGGGSDKTHTCPPCPAPELLGGPSVFLFPP |
| Fc_Knob_Cys_ | | | KPKDTLMISRTPEVTCVVVDVSHEDPEVKFNWYVDGVEVHNAKTKP |
| N297A | | | REEQYASTYRVVSVLTVLHQDWLNGKEYKCKVSNKALPAPIEKTIS |
| | | | KAKGQPREPQVYTLPPCREEMTKNQVSLWCLVKGFYPSDIAVEWES |
| | | | NGQPENNYKTTPPVLDSDGSFFLYSKLTVDKSRWQQGNVFSCSVMH |
| | | | EALHNHYTQKSLSLSPGK |
| | 3540 | IL-7 | DCDIEGKDGKQYESVLMVSIDQLLDSMKEIGSNCLNNEFNFFKRHIC |
| | | | DANKEGMFLFRAARKLRQFLKMNSTGDFDLHLLKVSEGTTILLNCT |
| | | | GQVKGRKPAALGEAQPTKSLEENKSLKEQKKLNDLCFLKRLLQEIKT |
| | | | CWNKILMGTKEH |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | 3518 | Full Sequence | DIQMTQSPSFLSASVGDRVTITCKASQNVADRVVWHQQKPGKAPKA |
| TCRVβ light | | | LIYSSSHRYKGVPSRFSGSGSGTEFTLTISSLQPEDFATYFCQQFKSYP |
| chain (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

BLM0319(IL-12)

| | | | |
|---|---|---|---|
| Chain 1: | 3517 | Full Sequence | QVQLVQSGAEVKKPGSSVKVSCKASGHDFRLTYIHWVRQAPGQGLE |
| TCRVβ- | | | WMGRVSAGSGNVKYNEKFKGRVTITADTSTSTAYMELSSLRSEDTA |
| Fc_Hole_Cys_ | | | VYYCAVSYYSYDVLDYWGQGTTVTVSSASTKGPSVFPLAPSSKSTS |
| N297A heavy | | | GGTAALGCLVKDYFPEPVTVSWNSGALTSGVHTFPAVLQSSGLYSLS |
| chain (CDRs | | | SVVTVPSSSLGTQTYICNVNHKPSNTKVDKRVEPKSCDKTHTCPPCP |
| underlined) | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2: | 3541 | Full Sequence | IWELKKDVYVVELDWYPDAPGEMVVLTCDTPEEDGITWTLDQSSEV |
| IL-12-GS | | | LGSGKTLTIQVKEFGDAGQYTCHKGGEVLSHSLLLLHKKEDGIWSTD |
| linker-IL15- | | | ILKDQKEPKNKTFLRCEAKNYSGRFTCWWLTTISTDLTFSVKSSRGSS |
| GS linker- | | | DPQGVTCGAATLSAERVRGDNKEYEYSVECQEDSACPAAEESLPIEV |
| Fc_Knob_Cys_ | | | MVDAVHKLKYENYTSSFFIRDIIKPDPPKNLQLKPLKNSRQVEVSWE |
| N297A | | | YPDTWSTPHSYFSLTFCVQVQGKSKREKKDRVFTDKTSATVICRKNA |
| | | | SISVRAQDRYYSSSWSEWASVPCSGGGGSGGGGSGGGGSRNLPVAT |
| | | | PDPGMFPCLHHSQNLLRAVSNMLQKARQTLEFYPCTSEEIDHEDITK |
| | | | DKTSTVEACLPLELTKNESCLNSRETSFITNGSCLASRKTSFMMALCL |
| | | | SSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNAS |
| | | | GGGGSGGGGSDKTHTCPPCPAPELLGGPSVFLFPPKPKDTLMISRTPE |
| | | | VTCVVVDVSHEDPEVKFNWYVDGVEVHNAKTKPREEQYASTYRVV |
| | | | SVLTVLHQDWLNGKEYKCKVSNKALPAPIEKTISKAKGQPREPQVY |
| | | | TLPPCREEMTKNQVSLWCLVKGFYPSDIAVEWESNGQPENNYKTTP |
| | | | PVLDSDGSFFLYSKLTVDKSRWQQGNVFSCSVMHEALHNHYTQKSL |
| | | | SLSPGK |
| | 3542 | IL-12 | IWELKKDVYVVELDWYPDAPGEMVVLTCDTPEEDGITWTLDQSSEV |
| | | | LGSGKTLTIQVKEFGDAGQYTCHKGGEVLSHSLLLLHKKEDGIWSTD |
| | | | ILKDQKEPKNKTFLRCEAKNYSGRFTCWWLTTISTDLTFSVKSSRGSS |
| | | | DPQGVTCGAATLSAERVRGDNKEYEYSVECQEDSACPAAEESLPIEV |
| | | | MVDAVHKLKYENYTSSFFIRDIIKPDPPKNLQLKPLKNSRQVEVSWE |
| | | | YPDTWSTPHSYFSLTFCVQVQGKSKREKKDRVFTDKTSATVICRKNA |
| | | | SISVRAQDRYYSSSWSEWASVPCSGGGGSGGGGSGGGGSRNLPVAT |
| | | | PDPGMFPCLHHSQNLLRAVSNMLQKARQTLEFYPCTSEEIDHEDITK |
| | | | DKTSTVEACLPLELTKNESCLNSRETSFITNGSCLASRKTSFMMALCL |
| | | | SSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNAS |
| | 3543 | IL-12 beta | IWELKKDVYVVELDWYPDAPGEMVVLTCDTPEEDGITWTLDQSSEV |
| | | subunit | LGSGKTLTIQVKEFGDAGQYTCHKGGEVLSHSLLLLHKKEDGIWSTD |
| | | | ILKDQKEPKNKTFLRCEAKNYSGRFTCWWLTTISTDLTFSVKSSRGSS |
| | | | DPQGVTCGAATLSAERVRGDNKEYEYSVECQEDSACPAAEESLPIEV |
| | | | MVDAVHKLKYENYTSSFFIRDIIKPDPPKNLQLKPLKNSRQVEVSWE |
| | | | YPDTWSTPHSYFSLTFCVQVQGKSKREKKDRVFTDKTSATVICRKNA |
| | | | SISVRAQDRYYSSSWSEWASVPC |
| | 3544 | Linker | SGGGGSGGGGSGGGGS |
| | 3545 | IL-12 alpha | RNLPVATPDPGMFPCLHHSQNLLRAVSNMLQKARQTLEFYPCTSEEI |
| | | subunit | DHEDITKDKTSTVEACLPLELTKNESCLNSRETSFITNGSCLASRKTSF |
| | | | MMALCLSSIYEDLKMYQVEFKTMNAKLLMDPKRQIFLDQNMLAVI |
| | | | DELMQALNENSETVPQKSSLEEPDFYKTKIKLCILLHAFRIRAVTIDR |
| | | | VMSYLNAS |
| | 3308 | linker | GGGGGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | 3518 | Full Sequence | DIQMTQSPSFLSASVGDRVTITCKASQNVADRVVWHQQKPGKAPKA |
| TCRVβ light | | | LIYSSSHRYKGVPSRFSGSGSGTEFTLTISSLQPEDFATYFCQQFKSYP |
| chain (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

BLM0321(IL-21)

| | | | |
|---|---|---|---|
| Chain 1: | 3517 | Full Sequence | QVQLVQSGAEVKKPGSSVKVSCKASGHDFRLTYIHWVRQAPGQGLE |
| TCRVβ- | | | WMGRVSAGSGNVKYNEKFKGRVTITADTSTSTAYMELSSLRSEDTA |
| Fc_Hole_Cys_ | | | VYYCAVSYYSYDVLDYWGQGTTVTVSSASTKGPSVFPLAPSSKSTS |
| N297A heavy | | | GGTAALGCLVKDYFPEPVTVSWNSGALTSGVHTFPAVLQSSGLYSLS |
| chain (CDRs | | | SVVTVPSSSLGTQTYICNVNHKPSNTKVDKRVEPKSCDKTHTCPPCP |
| underlined) | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSS |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2: | 3546 | Full Sequence | QGQDRHMIRMRQLIDIVDQLKNYVNDLVPEFLPAPEDVETNCEWSA |
| IL-21-GS | | | FSCFQKAQLKSANTGNNERIINVSIKKLKRKPPSTNAGRRQKHRLTCP |
| linker-IL15- | | | SCDSYEKKPPKEFLERFKSLLQKMIHQHLSSRTHGSEDSGGGGSGGG |
| GS linker- | | | GSDKTHTCPPCPAPELLGGPSVFLFPPKPKDTLMISRTPEVTCVVVDV |
| Fc_Knob_Cys_ | | | SHEDPEVKFNWYVDGVEVHNAKTKPREEQYASTYRVVSVLTVLHQ |
| N297A | | | DWLNGKEYKCKVSNKALPAPIEKTISKAKGQPREPQVYTLPPCREEM |
| | | | TKNQVSLWCLVKGFYPSDIAVEWESNGQPENNYKTTPPVLDSDGSFF |
| | | | LYSKLTVDKSRWQQGNVFSCSVMHEALHNHYTQKSLSLSPGK |
| | 2193 | IL-21 | QGQDRHMIRMRQLIDIVDQLKNYVNDLVPEFLPAPEDVETNCEWSA |
| | | | FSCFQKAQLKSANTGNNERIINVSIKKLKRKPPSTNAGRRQKHRLTCP |
| | | | SCDSYEKKPPKEFLERFKSLLQKMIHQHLSSRTHGSEDS |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | 3518 | Full Sequence | DIQMTQSPSFLSASVGDRVTITCKASQNVADRVVWHQQKPGKAPKA |
| TCRVβ light | | | LIYSSSHRYKGVPSRFSGSGSGTEFTLTISSLQPEDFATYFCQQFKSYP |
| chain (CDRs | | | LTFGQGTKLEIKRTVAAPSVFIFPPSDEQLKSGTASVVCLLNNFYPRE |
| underlined) | | | AKVQWKVDNALQSGNSQESVTEQDSKDSTYSLSSTLTLSKADYEKH |
| | | | KVYACEVTHQGLSSPVTKSFNRGEC |
| | 1349 | anti-TCRvβ VL | DIQMTQSPSFLSASVGDRVTITCKASQNVADRVVWHQQKPGKAPKA |
| | | | LIYSSSHRYKGVPSRFSGSGSGTEFTLTISSLQPEDFATYFCQQFKSYP |
| | | | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

Exemplary Multifunctional Molecule 1

| | | | |
|---|---|---|---|
| Chain 1: heavy | 3791 | Full Sequence | QVQLVQSGAEVKKPGSSVKVSCKASGHDFRLTYIHWVRQAPGQGLE |
| chain | | | WMGRVSAGSGNVKYNEKFKGRVTITADTSTSTAYMELSSLRSEDTA |
| | | | VYYCAVSYYSYDVLDYWGQGTTVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSP |
| | 591 | Full Sequence | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | (CDRs | WMGRVSAGSGNVKYNEKFKGRVTITADTSTSTAYMELSSLRSEDTA |
| | | underlined) | VYYCAVSYYSYDVLDYWGQGTTVTVSS |
| | 3792 | Human IgG1 | ASTKGPSVFPLAPSSKSTSGGTAALGCLVKDYFPEPVTVSWNSGALT |
| | | hole N297A | ISGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | 592 | Human IgG1 | ASTKGPSVFPLAPSSKSTSGGTAALGCLVKDYFPEPVTVSWNSGALT |
| | | hole N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2 | 3793 | Full Sequence | APTSSSTKKTQLQLEHLLLDLQMILNGINNYKNPKLTRMLTFKFYMP |
| (cytokine | | | KKATELKHLQCLEEELKPLEEVLNLAQSKNFHLRPRDLISNINVIVLE |
| containing | | | LKGSETTFMCEYADETATIVEFLNRWITFAQSIISTLTGGGGSGGGGS |
| chain) | | | DKTHTCPPCPAPELLGGPSVFLFPPKPKDTLMISRTPEVTCVVVDVSH |
| | | | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSP |
| | 593 | Full Sequence | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLTGGGGSGGGGS |
| | | | DKTHTCPPCPAPELLGGPSVFLFPPKPKDTLMISRTPEVTCVVVDVSH |
| | | | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| | 2270 | IL-2 C125A | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLT |
| | 3308 | linker | GGGGSGGGGS |
| | 3794 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSP |
| | 594 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3 (light | 3518 | Full Sequence | DIQMTQSPSFLSASVGDRVTITCKASQNVADRVVWHQQKPGKAPKA |
| chain) | | | LIYSSSHRYKGVPSRFSGSGSGTEFTLTISSLQPEDFATYFCQQFKSYP |
| | | | LTFGQGTKLEIKRTVAAPSVFIFPPSDEQLKSGTASVVCLLNNFYPRE |
| | | | AKVQWKVDNALQSGNSQESVTEQDSKDSTYSLSSTLTLSKADYEKH |
| | | | KVYACEVTHQGLSSPVTKSFNRGC |
| | 1349 | anti-TCRvβ VL | DIQMTQSPSFLSASVGDRVTITCKASQNVADRVVWHQQKPGKAPKA |
| | | (CDRs | LIYSSSHRYKGVPSRFSGSGSGTEFTLTISSLQPEDFATYFCQQFKSYP |
| | | underlined) | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

Exemplary Multifunctional Molecule 2

| | | | |
|---|---|---|---|
| Chain 1: heavy | 3795 | Full Sequence | QVQLVQSGAEVKKPGASVKVSCKASGFNIKSAYMHWVRQAPGQGL |
| chain | | | EWMGRIDPATGKTKYAPKFQARVTMTADTSTNTAYMELSSLRSEDT |
| | | | AVYYCARSLNWDYGLDYWGQGTLVTVSSASTKGPSVFPLAPSSKST |
| | | | SGGTAALGCLVKDYFPEPVTVSWNSGALTSGVHTFPAVLQSSGLYSL |
| | | | SSVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1113 | anti-TCRvβ VH | QVQLVQSGAEVKKPGASVKVSCKASGFNIKSAYMHWVRQAPGQGL |
| | | | EWMGRIDPATGKTKYAPKFQARVTMTADTSTNTAYMELSSLRSEDT |
| | | | AVYYCARSLNWDYGLDYWGQGTLVTVSS |
| | 1102 | HC CDR1 | SAYMH |
| | | (Kabat) | |
| | 1103 | HC CDR2 | RIDPATGKTKYAPKFQA |
| | | (Kabat) | |
| | 1104 | HC CDR3 | SLNWDYGLDY |
| | | (Kabat) | |
| | 1105 | HC CDR1 | GFNIKSA |
| | | (Chothia) | |
| | 1106 | HC CDR2 | DPATGK |
| | | (Chothia) | |
| | 1104 | HC CDR3 | SLNWDYGLDY |
| | | (Chothia) | |
| | 3640 | HC CDR1 | GFNIKSAYMH |
| | | (Combined) | |
| | 1103 | HC CDR2 | RIDPATGKTKYAPKFQA |
| | | (Combined) | |
| | 1104 | HC CDR3 | SLNWDYGLDY |
| | | (Combined) | |
| | 3649 | Human IgG1 | ASTKGPSVFPLAPSSKSTSGGTAALGCLVKDYFPEPVTVSWNSGALT |
| | | hole cys N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNQKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2 | 3793 | Full Sequence | APTSSSTKKTQLQLEHLLLDLQMILNGINNYKNPKLTRMLTFKFYMP |
| (cytokine | | | KKATELKHLQCLEEELKPLEEVLNLAQSKNFHLRPRDLISNINVIVLE |
| containing | | | LKGSETTFMCEYADETATIVEFLNRWITFAQSIISTLTGGGGSGGGGS |
| chain) | | | DKTHTCPPCPAPELLGGPSVFLFPPKPKDTLMISRTPEVTCVVVDVSH |
| | | | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSP |
| | 595 | Full Sequence | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLTGGGGSGGGGS |
| | | | DKTHTCPPCPAPELLGGPSVFLFPPKPKDTLMISRTPEVTCVVVDVSH |
| | | | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| | 2270 | IL-2 C125A | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLT |
| | 3308 | linker | GGGGSGGGGS |
| | 3794 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSP |
| | 596 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3 (light | 3796 | Full Sequence | EIVLTQSPGTLSLSPGERATLSCRASKSVSILGTHLIHWYQQKPGQAP |
| chain) | | | RLLIYAASNLESGIPDRFSGSGSETDFTLTISRLEPEDFAVYFCQQSIE |
| | | | DPWTFGGGTKVEIKRTVAAPSVFIFPPSDEQLKSGTASVVCLLNNFYPR |
| | | | EAKVQWKVDNALQSGNSQESVTEQDSKDSTYSLSSTLTLSKADYEK |
| | | | HKVYACEVTHQGLSSPVTKSFNRGEC |
| | 3797 | anti-TCRvβ VL | EIVLTQSPGTLSLSPGERATLSCRASKSVSILGTHLIHWYQQKPGQAP |
| | | | RLLIYAASNLESGIPDRFSGSGSETDFTLTISRLEPEDFAVYFCQQSIE |
| | | | DPWTFGGGTKVEIK |
| | 1107 | LC CDR1 | RASKSVSILGTHLIH |
| | | (Kabat) | |
| | 1108 | LC CDR2 | AASNLES |
| | | (Kabat) | |
| | 1109 | LC CDR3 | QQSIEDPWT |
| | | (Kabat) | |
| | 1110 | LC CDR1 | SKSVSILGTHL |
| | | (Chothia) | |
| | 1108 | LC CDR2 | AASNLES |
| | | (Chothia) | |
| | 1109 | LC CDR3 | QQSIEDPWT |
| | | (Chothia) | |
| | 1107 | LC CDR1 | RASKSVSILGTHLIH |
| | | (Combined) | |
| | 1108 | LC CDR2 | AASNLES |
| | | (Combined) | |
| | 1109 | LC CDR3 | QQSIEDPWT |
| | | (Combined) | |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLILSKADYEKHKVYACEVTHQGL |
| | | region | SSPVTKSFNRGEC |

Exemplary Multifunctional Molecule

| | | | |
|---|---|---|---|
| Chain 1: heavy | 3791 | Full Sequence | QVQLVQSGAEVKKPGSSVKVSCKASGHDFRLTYIHWVRQAPGQGLE |
| chain | | | WMGRVSAGSGNVKYNEKFKGRVTITADTSTSTAYMELSSLRSEDTA |
| | | | VYYCAVSYYSYDVLDYWGQGTTVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSP |
| | 597 | Full Sequence | QVQLVQSGAEVKKPGSSVKVSCKASGHDFRLTYIHWVRQAPGQGLE |
| | | | WMGRVSAGSGNVKYNEKFKGRVTITADTSTSTAYMELSSLRSEDTA |
| | | | VYYCAVSYYSYDVLDYWGQGTTVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVCTLPPSREEMTKNQVSLSCAVK |
| | | | GFYPSDIAVEWESNGQPENNYKTTPPVLDSDGSFFLVSKLTVDKSRW |
| | | | QQGNVFSCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | (CDRs | WMGRVSAGSGNVKYNEKFKGRVTITADTSTSTAYMELSSLRSEDTA |
| | | underlined) | VYYCAVSYYSYDVLDYWGQGTTVTVSS |
| | 3792 | Human IgG1 | ASTKGPSVFPLAPSSKSTSGGTAALGCLVKDYFPEPVTVSWNSGALT |
| | | hole N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | 598 | Human IgG1 | ASTKGPSVFPLAPSSKSTSGGTAALGCLVKDYFPEPVTVSWNSGALT |
| | | hole N297A | SGVHTFPAVLQSSGLYSLSSVVTVPSSSLGTQTYICNVNHKPSNTKVD |
| | | | KRVEPKSCDKTHTCPPCPAPELLGGPSVFLFPPKPKDTLMISRTPEVT |
| | | | CVVVDVSHEDPEVKFNWYVDGVEVHNAKTKPREEQYASTYRVVSV |
| | | | LTVLHQDWLNGKEYKCKVSNKALPAPIEKTISKAKGQPREPQVCTLP |
| | | | PSREEMTKNQVSLSCAVKGFYPSDIAVEWESNGQPENNYKTTPPVLD |
| | | | SDGSFFLVSKLTVDKSRWQQGNVFSCSVMHEALHNHYTQKSLSLSP |
| | | | GK |
| Chain 2 | 3798 | Full Sequence | ITCPPPMSVEHADIWVKSYSLYSRERYICNSGFKRKAGTSSLTECVLN |
| (cytokine | | | KATNVAHWTTPSLKCIRDPALVHQRPAPPSGGSGGGGSGGGSGGGG |
| containing | | | SGGGNWVNVISDLKKIEDLIQSMHIDATLYTESDVHPSCKVTAMKCF |
| chain) | | | LLELQVISLESGDASIHDTVENLIILANNSLSSNGNVTESGCKECEELE |
| | | | EKNIKEFLQSFVHIVQMFINTSGGGGSGGGGSDKTHTCPPCPAPELLG |
| | | | GPSVFLFPPKPKDTLMISRTPEVTCVVVDVSHEDPEVKFNWYVDGVE |
| | | | VHNAKTKPREEQYASTYRVVSVLTVLHQDWLNGKEYKCKVSNKAL |
| | | | PAPIEKTISKAKGQPREPQVYTLPPCREEMTKNQVSLWCLVKGFYPS |
| | | | DIAVEWESNGQPENNYKTTPPVLDSDGSFFLYSKLTVDKSRWQQGN |
| | | | VFSCSVMHEALHNHYTQKSLSLSPGK |
| | 3799 | IL-15 | ITCPPPMSVEHADIWVKSYSLYSRERYICNSGFKRKAGTSSLTECVLN |
| | | | KATNVAHWTTPSLKCIRDPALVHQRPAPPSGGSGGGGSGGGSGGGG |
| | | | SGGGNWVNVISDLKKIEDLIQSMHIDATLYTESDVHPSCKVTAMKCF |
| | | | LLELQVISLESGDASIHDTVENLIILANNSLSSNGNVTESGCKECEELE |
| | | | EKNIKEFLQSFVHIVQMFINTS |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3 (light | 3518 | Full Sequence | DIQMTQSPSFLSASVGDRVTITCKASQNVADRVVWHQQKPGKAPKA |
| chain) | | | LIYSSSHRYKGVPSRFSGSGSGTEFTLTISSLQPEDFATYFCQQFKSYP |
| | | | LTFGQGTKLEIKRTVAAPSVFIFPPSDEQLKSGTASVVCLLNNFYPRE |
| | | | AKVQWKVDNALQSGNSQESVTEQDSKDSTYSLSSTLTLSKADYEKH |
| | | | KVYACEVTHQGLSSPVTKSENRGEC |
| | 1349 | anti-TCRvβ VL | DIQMTQSPSFLSASVGDRVTITCKASQNVADRVVWHQQKPGKAPKA |
| | | (CDRs | LIYSSSHRYKGVPSRFSGSGSGTEFTLTISSLQPEDFATYFCQQFKSYP |
| | | underlined) | LTFGQGTKLEIK |
| | 3644 | Immunoglobulin | RTVAAPSVFIFPPSDEQLKSGTASVVCLLNNFYPREAKVQWKVDNAL |
| | | kappa constant C | QSGNSQESVTEQDSKDSTYSLSSTLTLSKADYEKHKVYACEVTHQGL |
| | | region | SSPVTKSFNRGEC |

Exemplary Multifunction Molecule 4

| | | | |
|---|---|---|---|
| Chain 1: | | | |
| Tumor | | | |
| targeting | | | |
| moiety heavy | | | |
| chain | | | |
| Chain 2: Anti- | 3800 | Full Sequence | QVQLVQSGAEVKKPGSSVKVSCKASGHDFRLTYIHWVRQAPGQGLE |
| TCRvβ 6 and | | | WMGRVSAGSGNVKYNEKFKGRVTITADTSTSTAYMELSSLRSEDTA |
| cytokine | | | VYYCAVSYYSYDVLDYWGQGTTVTVSSGGGGSGGGGSGGGGSGG |
| containing | | | GGSDIQMTQSPSFLSASVGDRVTITCKASQNVADRVVWHQQKPGKA |
| chain | | | PKALIYSSSHRYKGVPSRFSGSGSGTEFTLTISSLQPEDFATYFCQQFK |
| | | | SYPLTFGQGTKLEIKGGGGSGGGGSGGGGSAPTSSSTKKTQLQLEHL |
| | | | LLDLQMILNGINNYKNPKLTRMLTFKFYMPKKATELKHLQCLEEEL |
| | | | KPLEEVLNLAQSKNFHLRPRDLISNINVIVLELKGSETTFMCEYADET |
| | | | ATIVEFLNRWITFAQSIISTLTGGGGSGGGGSDKTHTCPPCPAPELLGG |
| | | | PSVFLFPPKPKDTLMISRTPEVTCVVVDVSHEDPEVKFNWYVDGVEV |
| | | | HNAKTKPREEQYASTYRVVSVLIVLHQDWLNGKEYKCKVSNKALP |
| | | | APIEKTISKAKGQPREPQVYTLPPCREEMTKNQVSLWCLVKGFYPSDI |
| | | | AVEWESNGQPENNYKTTPPVLDSDGSFFLYSKLTVDKSRWQQGNVF |
| | | | SCSVMHEALHNHYTQKSLSLSPGK |
| | 1346 | anti-TCRvβ VH | QVQLVQSGAEVKKPGSSVKVSCKASGHDFRLTYIHWVRQAPGQGLE |
| | | (CDRs | WMGRVSAGSGNVKYNEKFKGRVTITADTSTSTAYMELSSLRSEDTA |
| | | underlined) | VYYCAVSYYSYDVLDYWGQGTTVTVSS |
| | 3801 | linker | GGGGSGGGGSGGGGSGGGGS |
| | 1349 | anti-TCRvβ VL | DIQMTQSPSFLSASVGDRVTITCKASQNVADRVVWHQQKPGKAPKA |
| | | (CDRs | LIYSSSHRYKGVPSRFSGSGSGTEFTLTISSLQPEDFATYFCQQFKSYP |
| | | underlined) | LTFGQGTKLEIK |
| | 3309 | linker | GGGGSGGGGSGGGGS |
| | 2270 | IL-2 C125A | APTSSSTKKTQLQLEHLLLDLQMILNGINNYKNPKLTRMLTFKFYMP |
| | | | KKATELKHLQCLEEELKPLEEVLNLAQSKNFHLRPRDLISNINVIVLE |
| | | | LKGSETTFMCEYADETATIVEFLNRWITFAQSIISTLT |
| | 3308 | linker | GGGGSGGGGS |
| | 3648 | Human Fc Knob | DKTHTCPPCPAPELLGGPSVFLFPPKPKDTLMISRTPEVTCVVVDVSH |
| | | cys N297A | EDPEVKFNWYVDGVEVHNAKTKPREEQYASTYRVVSVLTVLHQDW |
| | | | LNGKEYKCKVSNKALPAPIEKTISKAKGQPREPQVYTLPPCREEMTK |
| | | | NQVSLWCLVKGFYPSDIAVEWESNGQPENNYKTTPPVLDSDGSFFLY |
| | | | SKLTVDKSRWQQGNVFSCSVMHEALHNHYTQKSLSLSPGK |
| Chain 3: | | | |
| Tumor | | | |
| targeting | | | |
| moiety light | | | |
| chain | | | |

TABLE 22
Exemplary Construct Sequences

| | | | |
|---|---|---|---|
| | | SEQ | |
| Polypeptide | | ID | |
| Chain | Component | NO: | Sequence |

BLM0155

| | | | |
|---|---|---|---|
| Polypeptide | Full | 501 | QVQLVQSGAEVKKPGSSVKVSCKASGYSFTGYTMNWVRQAPG |
| Chain 1 | Sequence | | QGLEWMGLITPYNGASSYNQKFRGRVTITVDKSTSTAYMELSSL |
| Hole | | | RSEDTAVYYCARGGYDGRGFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKRV |
| | | | EPKSCDKTHTCPPCPAPELLGGPSVFLFPPKPKDTLMISRTPEVTC |
| | | | VVVDVSHEDPEVKFNWYVDGVEVHNAKTKPREEQYASTYRVV |
| | | | SVLTVLHQDWLNGKEYKCKVSNKALPAPIEKTISKAKGQPREPQ |
| | | | VCTLPPSREEMTKNQVSLSCAVKGFYPSDIAVEWESNGQPENNY |
| | | | KTTPPVLDSDGSFFLVSKLTVDKSRWQQGNVFSCSVMHEALHN |
| | | | HYTQKSLSLSPGK |
| | VH 1 | 523 | QVQLVQSGAEVKKPGSSVKVSCKASGYSFTGYTMNWVRQAPG |
| | Anti-MSLN | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 3649 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Hole | | QYASTYRVVSVLTVLHQDWLNGKEYKCKVSNKALPAPIEKTIS |
| | N297A | | KAKGQPREPQVCTLPPSREEMTKNQVSLSCAVKGFYPSDIAVEW |
| | | | ESNGQPENNYKTTPPVLDSDGSFFLVSKLTVDKSRWQQGNVFSC |
| | | | SVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 550 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Hole | | VLHQDWLNGKEYKCKVSNKALPAPIEKTISKAKGQPREPQVCT |
| | | | LPPSREEMTKNQVSLSCAVKGFYPSDIAVEWESNGQPENNYKTT |
| | | | PPVLDSDGSFFLVSKLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 3 | Sequence | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 1 | 527 | EIVLTQSPATLSLSPGERATLSCSASSSVSYMHWYQQKPGLAPRR |
| | Anti-MSLN | | LIYDTSKLASGIPDRFSGSGSQNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 3800 | QVQLVQSGAEVKKPGSSVKVSCKASGHDFRLTYIHWVRQAPGQ |
| Chain 2 | Sequence | | GLEWMGRVSAGSGNVKYNEKFKGRVTITADTSTSTAYMELSSL |
| Knob | | | RSEDTAVYYCAVSYYSYDVLDYWGQGTTVTVSSGGGGSGGGG |
| Anti-TCRvβ6 | | | SGGGGSGGGGSDIQMTQSPSFLSASVGDRVTITCKASQNVADRV |
| and | | | VWHQQKPGKAPKALIYSSSHRYKGVPSRFSGSGSGTEFTLTISSL |
| cytokine | | | QPEDFATYFCQQFKSYPLTFGQGTKLEIKGGGGSGGGGSGGGGS |
| containing | | | APTSSSTKKTQLQLEHLLLDLQMILNGINNYKNPKLTRMLTFKF |
| chain | | | YMPKKATELKHLQCLEEELKPLEEVLNLAQSKNFHLRPRDLISNI |
| | | | NVIVLELKGSETTFMCEYADETATIVEFLNRWITFAQSIISTLTGG |
| | | | GGSGGGGSDKTHTCPPCPAPELLGGPSVFLFPPKPKDTLMISRTP |
| | | | EVTCVVVDVSHEDPEVKFNWYVDGVEVHNAKTKPREEQYAST |
| | | | YRVVSVLTVLHQDWLNGKEYKCKVSNKALPAPIEKTISKAKGQ |
| | | | PREPQVYTLPPCREEMTKNQVSLWCLVKGFYPSDIAVEWESNG |
| | | | QPENNYKTTPPVLDSDGSFFLYSKLTVDKSRWQQGNVFSCSVM |
| | | | HEALHNHYTQKSLSLSPGK |
| | scFv | 1331 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIK |
| | VH 2 | 1346 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSS |
| | HC CDR1 | 3650 | GHDFRLTYIH |
| | HC CDR2 | 3651 | RVSAGSGNVKYNEKFKG |
| | HC CDR3 | 5 | SYYSYDVLDY |
| | Linker 1 | 3801 | GGGGSGGGGSGGGGSGGGGS |
| | VL 2 | 1349 | DIQMTQSPSFLSASVGDRVTITCKASQNVADRVVWHQQKPGKA |
| | | | PKALIYSSSHRYKGVPSRFSGSGSGTEFTLTISSLQPEDFATYFCQ |
| | | | QFKSYPLTFGQGTKLEIK |
| | LC CDR1 | 3655 | KASQNYADRVV |
| | LC CDR2 | 3653 | SSSHRYK |
| | LC CDR3 | 8 | QQFKSYPLT |
| | Linker 2 | 3309 | GGGGSGGGGSGGGGS |
| | IL-2 C125A | 2270 | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFAQSIISTLT |
| | Linker 3 | 3308 | GGGGSGGGGS |
| | Fc (CH2- | 3648 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Knob | | VLHQDWLNGKEYKCKVSNKALPAPIEKTISKAKGQPREPQVYT |
| | N297A | | LPPCREEMTKNQVSLWCLVKGFYPSDIAVEWESNGQPENNYKT |
| | | | TPPVLDSDGSFFLYSKLTVDKSRWQQGNVFSCSVMHEALHNHY |
| | | | TQKSLSLSPGK |

BMM0462

| | | | |
|---|---|---|---|
| Polypeptide | Full | 590 | QVQLVQSGAEVKKPGSSVKVSCKASGYSFTGYTMNWVRQAPG |
| Chain 1 | Sequence | | QGLEWMGLITPYNGASSYNQKFRGRVTITVDKSTSTAYMELSSL |
| Hole | | | RSEDTAVYYCARGGYDGRGFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKKV |
| | | | EPKSCDGGGGSGGGGSQVQLVQSGAEVKKPGSSVKVSCKASGY |
| | | | SFTGYTMNWVRQAPGQGLEWMGLITPYNGASSYNQKFRGRVTI |
| | | | TVDKSTSTAYMELSSLRSEDTAVYYCARGGYDGRGFDYWGQG |
| | | | TLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYIC |
| | | | NVNHKPSNTKVDKRVEPKSCDKTHTCPPCPAPELLGGPSVFLFP |
| | | | PKPKDTLMISRTPEVTCVVVDVSHEDPEVKFNWYVDGVEVHNA |
| | | | KTKPREEQYASTYRVVSVLTVLHQDWLNGKEYKCKVSNKALP |
| | | | APIEKTISKAKGQPREPQVCTLPPSREEMTKNQVSLSCAVKGFYP |
| | | | SDIAVEWESNGQPENNYKTTPPVLDSDGSFFLVSKLTVDKSRWQ |
| | | | QGNVFSCSVMHEALHNHYTQKSLSLSPGK |
| | VH 1 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | CH1 | 551 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKKVEPKSCD |
| | Linker 1 | 3308 | GGGGSGGGGS |
| | VH 2 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 3649 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Hole | | QYASTYRVVSVLTVLHQDWLNGKEYKCKVSNKALPAPIEKTIS |
| | N297A | | KAKGQPREPQVCTIPPSREEMTKNQVSLSCAVKGFYPSDIAVEW |
| | | | ESNGQPENNYKTTPPVLDSDGSFFLVSKLTVDKSRWQQGNVFSC |
| | | | SVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 550 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Hole | | VLHQDWLNGKEYKCKVSNKALPAPIEKTISKAKGQPREPQVCT |
| | | | LPPSREEMTKNQVSLSCAVKGFYPSDIAVEWESNGQPENNYKTT |
| | | | PPVLDSDGSFFLVSKLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 3 | Sequence | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 1 or 2 | 527 | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 3800 | QVQLVQSGAEVKKPGSSVKVSCKASGHDFRLTYIHWVRQAPGQ |
| Chain 2 | Sequence | | GLEWMGRVSAGSGNVKYNEKFKGRVTITADTSTSTAYMELSSL |
| Knob | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIKGGGGSGGGGSGGGGS |
| | | | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFAQSIISTLTGG |
| | | | GGSGGGGSDKTHTCPPCPAPELLGGPSVFLFPPKPKDTLMISRTP |
| | | | EVTCVVVDVSHEDPEVKFNWYVDGVEVHNAKTKPREEQYAST |
| | | | YRVVSVLTVLHQDWLNGKEYKCKVSNKALPAPIEKTISKAKGQ |
| | | | PREPQVYTLPPCREEMTKNQVSLWCLVKGFYPSDIAVEWESNG |
| | | | QPENNYKTTPPVLDSDGSFFLYSKLTVDKSRWQQGNVFSCSVM |
| | | | HEALHNHYTQKSLSLSPGK |
| | scFv | 1331 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIK |
| | VH 3 | 1346 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSS |
| | HC CDR1 | 3650 | GHDFRLTYIH |
| | HC CDR2 | 3651 | RVSAGSGNVKYNEKFKG |
| | HC CDR3 | 5 | SYYSYDVLDY |
| | Linker 2 | 3801 | GGGGSGGGGSGGGGSGGGGS |
| | VL 3 | 1349 | DIQMTQSPSFLSASVGDRVTITCKASQNVADRVVWHQQKPGKA |
| | | | PKALIYSSSHRYKGVPSRFSGSGSGTEFTLTISSLQPEDFATYFCQ |
| | | | QFKSYPLTFGQGTKLEIK |
| | LC CDR1 | 3655 | KASQNVADRVV |
| | LC CDR2 | 3653 | SSSHRYK |
| | LC CDR3 | 8 | QQFKSYPLT |
| | Linker 3 | 3309 | GGGGSGGGGSGGGGS |
| | IL-2 C125A | 2270 | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFAQSIISTLT |
| | Linker 4 | 3308 | GGGGSGGGGS |
| | Fc (CH2- | 3648 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Knob | | VLHQDWLNGKEYKCKVSNKALPAPIEKTISKAKGQPREPQVYT |
| | N297A | | LPPCREEMTKNQVSLWCLVKGFYPSDIAVEWESNGQPENNYKT |
| | | | TPPVLDSDGSFFLYSKLTVDKSRWQQGNVFSCSVMHEALHNHY |
| | | | TQKSLSLSPGK |

BLM0158

| | | | |
|---|---|---|---|
| Polypeptide | Full | 501 | QVQLVQSGAEVKKPGSSVKVSCKASGYSFTGYTMNWVRQAPG |
| Chain 1 | Sequence | | QGLEWMGLITPYNGASSYNQKFRGRVTITVDKSTSTAYMELSSL |
| Hole | | | RSEDTAVYYCARGGYDGRGFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKRV |
| | | | EPKSCDKTHTCPPCPAPELLGGPSVFLFPPKPKDTLMISRTPEVTC |
| | | | VVVDVSHEDPEVKFNWYVDGVEVHNAKTKPREEQYASTYRVV |
| | | | SVLTVLHQDWLNGKEYKCKVSNKALPAPIEKTISKAKGQPREPQ |
| | | | VCTLPPSREEMTKNQVSLSCAVKGFYPSDIAVEWESNGQPENNY |
| | | | KTTPPVLDSDGSFFLVSKLTVDKSRWQQGNVFSCSVMHEALHN |
| | | | HYTQKSLSLSPGK |
| | VH 1 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 3649 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Hole | | QYASTYRVVSVLTVLHQDWLNGKEYKCKVSNKALPAPIEKTIS |
| | N297A | | KAKGQPREPQVCTLPPSREEMTKNQVSLSCAVKGFYPSDIAVEW |
| | | | ESNGQPENNYKTTPPVLDSDGSFFLVSKLTVDKSRWQQGNVFSC |
| | | | SVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 550 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Hole | | VLHQDWLNGKEYKCKVSNKALPAPIEKTISKAKGQPREPQVCT |
| | | | LPPSREEMTKNQVSLSCAVKGFYPSDIAVEWESNGQPENNYKTT |
| | | | PPVLDSDGSFFLVSKLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 3 | Sequence | | LIYDISKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 1 | 527 | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 503 | QVQLVQSGAEVKKPGSSVKVSCKASGHDFRLTYIHWVRQAPGQ |
| Chain 2 | Sequence | | GLEWMGRVSAGSGNVKYNEKFKGRVTITADTSTSTAYMELSSL |
| Knob | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIKGGGGSGGGGSGGGGS |
| | | | DKTHTCPPCPAPELLGGPSVFLFPPKPKDTLMISRTPEVTCVVVD |
| | | | VSHEDPEVKFNWYVDGVEVHNAKTKPREEQYASTYRVVSVLT |
| | | | VLHQDWLNGKEYKCKVSNKALPAPIEKTISKAKGQPREPQVYT |
| | | | LPPCREEMTKNQVSLWCLVKGFYPSDIAVEWESNGQPENNYKT |
| | | | TPPVLDSDGSFFLYSKLTVDKSRWQQGNVFSCSVMHEALHNHY |
| | | | TQKSLSLSPGK |
| | scFv | 1331 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIK |
| | VH 2 | 1346 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSS |
| | HC CDR1 | 3650 | GHDFRLTYIH |
| | HC CDR2 | 3651 | RVSAGSGNVKYNEKFKG |
| | HC CDR3 | 5 | SYYSYDVLDY |
| | Linker 1 | 3801 | GGGGSGGGGSGGGGSGGGGS |
| | VL 2 | 1349 | DIQMTQSPSFLSASVGDRVTITCKASQNVADRVVWHQQKPGKA |
| | | | PKALIYSSSHRYKGVPSRFSGSGSGTEFTLTISSLQPEDFATYFCQ |
| | | | QFKSYPLTFGQGTKLEIK |
| | LC CDR1 | 3655 | KASQNVADRVV |
| | LC CDR2 | 3653 | SSSHRYK |
| | LC CDR3 | 8 | QQFKSYPLT |
| | Linker 2 | 3309 | GGGGSGGGGSGGGGS |
| | Fc (CH2- | 3648 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Knob | | VLHQDWLNGKEYKCKVSNKALPAPIEKTISKAKGQPREPQVYT |
| | N297A | | LPPCREEMTKNQVSLWCLVKGFYPSDIAVEWESNGQPENNYKT |
| | | | TPPVLDSDGSFFLYSKLTVDKSRWQQGNVFSCSVMHEALHNHY |
| | | | TQKSLSLSPGK |

BMM0274

| | | | |
|---|---|---|---|
| Polypeptide | Full | 504 | QVQLVQSGAEVKKPGSSVKVSCKASGHDFRLTYIHWVRQAPGQ |
| Chain 1 | Sequence | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIKGGGGSQVQLVQSGAE |
| | | | VKKPGSSVKVSCKASGYSFTGYTMNWVRQAPGQGLEWMGLIT |
| | | | PYNGASSYNQKFRGRVTITVDKSTSTAYMELSSLRSEDTAVYYC |
| | | | ARGGYDGRGFDYWGQGTLVTVSSASTKGPSVFPLAPSSKSTSG |
| | | | GTAALGCLVKDYFPEPVTVSWNSGALTSGVHTFPAVLQSSGLYS |
| | | | LSSVVTVPSSSLGTQTYICNVNHKPSNTKVDKKVEPKSCDKTHT |
| | | | CPPCPAPELLGGPSVFLFPPKPKDTLMISRTPEVTCVVVDVSHED |
| | | | PEVKFNWYVDGVEVHNAKTKPREEQYASTYRVVSVLTVLHQD |
| | | | WLNGKEYKCKVSNKALPAPIEKTISKAKGQPREPQVYTLPPSRE |
| | | | EMTKNQVSLTCLVKGFYPSDIAVEWESNGQPENNYKTTPPVLDS |
| | | | DGSFFLYSKLTVDKSRWQQGNVFSCSVMHEALHNHYTQKSLSL |
| | | | SPGK |
| | scFv | 1331 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIK |
| | VH 1 | 1346 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSS |
| | HC CDR1 | 3650 | GHDFRLTYIH |
| | HC CDR2 | 3651 | RVSAGSGNVKYNEKFKG |
| | HC CDR3 | 5 | SYYSYDVLDY |
| | Linker 1 | 3801 | GGGGSGGGGSGGGGSGGGGS |
| | VL 1 | 1349 | DIQMTQSPSFLSASVGDRVTITCKASQNVADRVVWHQQKPGKA |
| | | | PKALIYSSSHRYKGVPSRFSGSGSGTEFTLTISSLQPEDFATYFCQ |
| | | | QFKSYPLTFGQGTKLEIK |
| | LC CDR1 | 3655 | KASQNVADRVV |
| | LC CDR2 | 3653 | SSSHRYK |
| | LC CDR3 | 8 | QQFKSYPLT |
| | Linker 2 | 3307 | GGGGS |
| | VH 2 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 3645 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKKVEPKSCDKTHTCPPCPAPELLGGPSVFLFPPKPKDTL |
| | Region | | MISRTPEVTCVVVDVSHEDPEVKFNWYVDGVEVHNAKTKPREE |
| | | | QYASTYRVVSVLTVLHQDWLNGKEYKCKVSNKALPAPIEKTIS |
| | | | KAKGQPREPQVYTLPPSREEMTKNQVSLTCLVKGFYPSDIAVEW |
| | | | ESNGQPENNYKTTPPVLDSDGSFFLYSKLTVDKSRWQQGNVFSC |
| | | | SVMHEALHNHYTQKSLSLSPGK |
| | CH1 | 552 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKKVEPKSC |
| | Fc (CH2- | 553 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | | | VLHQDWLNGKEYKCKVSNKALPAPIEKTISKAKGQPREPQVYT |
| | | | LPPSREEMTKNQVSLTCLVKGFYPSDIAVEWESNGQPENNYKTT |
| Polypeptide | Full | 502 | PPVLDSDGSFFLYSKLTVDKSRWQQGNVFSCSVMHEALHNHYT |
| Chain 3 | Sequence | | QKSLSLSPGK |
| | | | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 2 | 527 | EIVLTQSPATLSLSPGERATLSCSASSSVSYMHWYQQKPGLAPRR |
| | | | LIYDTSKLASGIPDRFSGSGSQNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 504 | QVQLVQSGAEVKKPGSSVKVSCKASGHDFRLTYIHWVRQAPGQ |
| Chain 2 | Sequence | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIKGGGGSQVQLVQSGAE |
| | | | VKKPGSSVKVSCKASGYSFTGYTMNWVRQAPGQGLEWMGLIT |
| | | | PYNGASSYNQKFRGRVTITVDKSTSTAYMELSSLRSEDTAVYYC |
| | | | ARGGYDGRGFDYWGQGTLVTVSSASTKGPSVFPLAPSSKSTSG |
| | | | GTAALGCLVKDYFPEPVTVSWNSGALTSGVHTFPAVLQSSGLYS |
| | | | LSSVVTVPSSSLGTQTYICNVNHKPSNTKVDKKVEPKSCDKTHT |
| | | | CPPCPAPELLGGPSVFLFPPKPKDTLMISRTPEVTCVVVDVSHED |
| | | | PEVKFNWYVDGVEVHNAKTKPREEQYASTYRVVSVLTVLHQD |
| | | | WLNGKEYKCKVSNKALPAPIEKTISKAKGQPREPQVYTLPPSRE |
| | | | EMTKNQVSLTCLVKGFYPSDIAVEWESNGQPENNYKTTPPVLDS |
| | | | DGSFFLYSKLTVDKSRWQQGNVFSCSVMHEALHNHYTQKSLSL |
| | | | SPGK |
| | scFv | 1331 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSSGGGGSGGGG |
| | | | SGGGGSGGGGSDIQMTQSPSFLSASVGDRVTITCKASQNVADRV |
| | | | VWHQQKPGKAPKALIYSSSHRYKGVPSRFSGSGSGTEFTLTISSL |
| | | | QPEDFATYFCQQFKSYPLTFGQGTKLEIK |
| | VH 3 | 1346 | QVQLVQSGAEVKKPGSSVKVSCKASGHDFRLTYIHWVRQAPGQ |
| | | | GLEWMGRVSAGSGNVKYNEKFKGRVTITADTSTSTAYMELSSL |
| | | | RSEDTAVYYCAVSYYSYDVLDYWGQGTTVTVSS |
| | HC CDR1 | 3650 | GHDFRLTYIH |
| | HC CDR2 | 3651 | RVSAGSGNVKYNEKFKG |
| | HC CDR3 | 5 | SYYSYDVLDY |
| | Linker 3 | 3801 | GGGGSGGGGSGGGGSGGGGS |
| | VL 3 | 1349 | DIQMTQSPSFLSASVGDRVTITCKASQNVADRVVWHQQKPGKA |
| | | | PKALIYSSSHRYKGVPSRFSGSGSGTEFTLTISSLQPEDFATYFCQ |
| | | | QFKSYPLTFGQGTKLEIK |
| | LC CDR1 | 3655 | KASQNVADRVV |
| | LC CDR2 | 3653 | SSSHRYK |
| | LC CDR3 | 8 | QQFKSYPLT |
| | Linker 4 | 3307 | GGGGS |
| | VH 4 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 3645 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKKVEPKSCDKTHTCPPCPAPELLGGPSVFLFPPKPKDTL |
| | Region | | MISRTPEVTCVVVDVSHEDPEVKFNWYVDGVEVHNAKTKPREE |
| | | | QYASTYRVVSVLTVLHQDWLNGKEYKCKVSNKALPAPIEKTIS |
| | | | KAKGQPREPQVYTLPPSREEMTKNQVSLTCLVKGFYPSDIAVEW |
| | | | ESNGQPENNYKTTPPVLDSDGSFFLYSKLTVDKSRWQQGNVFSC |
| | | | SVMHEALHNHYTQKSLSLSPGK |
| | CH1 | 552 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKKVEPKSC |
| | Fc (CH2- | 553 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | | | VLHQDWLNGKEYKCKVSNKALPAPIEKTISKAKGQPREPQVYT |
| | | | LPPSREEMTKNQVSLTCLVKGFYPSDIAVEWESNGQPENNYKTT |
| | | | PPVLDSDGSFFLYSKLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 4 | Sequence | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 4 | 527 | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |

BMM0388

| | | | |
|---|---|---|---|
| Polypeptide | Full | 505 | QVQLVQSGAEVKKPGSSVKVSCKASGYSFTGYTMNWVRQAPG |
| Chain 1 | Sequence | | QGLEWMGLITPYNGASSYNQKFRGRVTITVDKSTSTAYMELSSL |
| Knob | | | RSEDTAVYYCARGGYDGRGFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKRV |
| | | | EPKSCDKTHTCPPCPAPELLGGPSVFLFPPKPKDTLMISRTPEVTC |
| | | | VVVDVSHEDPEVKFNWYVDGVEVHNAKTKPREEQYASTYRVV |
| | | | SVLTVLHQDWLNGKEYKCKVSNKALPAPIEKTISKAKGQPREPQ |
| | | | VYTLPPSRDELTKNQVSLTCLVKGFYPSDIAVEWESNGQPENNY |
| | | | KTTPPVLDSDGSFFLYSRLTVDKSRWQQGNVFSCSVMHEALHN |
| | | | HYTQKSLSLSPGK |
| | VH 1 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 554 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Knob | | QYASTYRVVSVLTVLHQDWLNGKEYKCKVSNKALPAPIEKTIS |
| | | | KAKGQPREPQVYTLPPSRDELTKNQVSLTCLVKGFYPSDIAVEW |
| | | | ESNGQPENNYKTTPPVLDSDGSFFLYSRLTVDKSRWQQGNVFSC |
| | | | SVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 555 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Knob | | VLHQDWLNGKEYKCKVSNKALPAPIEKTISKAKGQPREPQVYT |
| | | | LPPSRDELTKNQVSLTCLVKGFYPSDIAVEWESNGQPENNYKTT |
| | | | PPVLDSDGSFFLYSRLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 3 | Sequence | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 1 | 527 | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 506 | EVKLVESGGGLVQPGGSLRLSCAASGFTFNTYAMNWVRQAPG |
| Chain 2 | Sequence | | KGLEWVARIRSKYNNYATYYADSVKDRFTISRDDSKSSLYLQM |
| Hole | | | NNLKTEDTAMYYCVRHGNFGNSYVSWFAYWGQGTLVTVSSAS |
| | | | TKGPSVFPLAPSSKSTSGGTAALGCLVKDYFPEPVTVSWNSGAL |
| | | | TSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQ |
| | | | YASTYRVVSVLTVLHQDWLNGKEYKCKVSNKALPAPIEKTISK |
| | | | AKGQPREPQVYTLPPSRDELTKNQVSLTCLVKGFYPSDIAVEWE |
| | | | SNGQPENNYKTTPPVLDSDGSFLLYSKLTVDKSRWQQGNVFSCS |
| | | | VMHEALHNHYTQKSLSLSPGK |
| | VH 2 | 529 | EVKLVESGGGLVQPGGSLRLSCAASGFTFNTYAMNWVRQAPG |
| | | | KGLEWVARIRSKYNNYATYYADSVKDRFTISRDDSKSSLYLQM |
| | | | NNLKTEDTAMYYCVRHGNFGNSYVSWFAYWGQGTLVTVSS |
| | HC CDR1 | 530 | GFTFNTYAMN |
| | HC CDR2 | 531 | RIRSKYNNYATYYADSVKD |
| | HC CDR3 | 532 | HGNFGNSYVSWFAY |
| | Heavy | 556 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Hole | | QYASTYRVVSVLTVLHQDWLNGKEYKCKVSNKALPAPIEKTIS |
| | | | KAKGQPREPQVYTLPPSRDELTKNQVSLTCLVKGFYPSDIAVEW |
| | | | ESNGQPENNYKTTPPVLDSDGSFLLYSKLTVDKSRWQQGNVFS |
| | | | CSVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 557 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Hole | | VLHQDWLNGKEYKCKVSNKALPAPIEKTISKAKGQPREPQVYT |
| | | | LPPSRDELTKNQVSLTCLVKGFYPSDIAVEWESNGQPENNYKTT |
| | | | PPVLDSDGSFLLYSKLTVDKSRWQQGNVFSCSVMHEALHNHYT |
| | | | QKSLSLSPGK |
| Polypeptide | Full | 507 | QAVVTQEPSFSVSPGGTVTLTCRSSTGAVTTSNYANWVQQTPG |
| Chain 4 | Sequence | | QAFRGLIGGTNKRAPGVPARFSGSLIGDKAALTITGAQADDESIY |
| | | | FCALWYSNLWVFGGGTKLTVLGQPKANPTVTLFPPSSEELQAN |
| | | | KATLVCLISDFYPGAVTVAWKADGSPVKAGVETTKPSKQSNNK |
| | | | YAASSYLSLTPEQWKSHRSYSCQVTHEGSTVEKTVAPTECS |
| | VL 2 | 533 | QAVVTQEPSFSVSPGGTVTLTCRSSTGAVTTSNYANWVQQTPG |
| | | | QAFRGLIGGTNKRAPGVPARFSGSLIGDKAALTITGAQADDESIY |
| | | | FCALWYSNLWVFGGGTKLTVL |
| | LC CDR1 | 534 | RSSTGAVTTSNYAN |
| | LC CDR2 | 535 | GTNKRAP |
| | LC CDR3 | 536 | ALWYSNLWV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |

BMM0466

| | | | |
|---|---|---|---|
| Polypeptide | Full | 508 | QVQLVQSGAEVKKPGSSVKVSCKASGYSFTGYTMNWVRQAPG |
| Chain 1 | Sequence | | QGLEWMGLITPYNGASSYNQKFRGRVTITVDKSTSTAYMELSSL |
| Knob | | | RSEDTAVYYCARGGYDGRGFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKKV |
| | | | EPKSCDGGGGSGGGGSQVQLVQSGAEVKKPGSSVKVSCKASGY |
| | | | SFTGYTMNWVRQAPGQGLEWMGLITPYNGASSYNQKFRGRVTI |
| | | | TVDKSTSTAYMELSSLRSEDTAVYYCARGGYDGRGFDYWGQG |
| | | | TLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYIC |
| | | | NVNHKPSNTKVDKRVEPKSCDKTHTCPPCPAPELLGGPSVFLFP |
| | | | PKPKDTLMISRTPEVTCVVVDVSHEDPEVKFNWYVDGVEVHNA |
| | | | KTKPREEQYASTYRVVSVLTVLHQDWLNGKEYKCKVSNKALP |
| | | | APIEKTISKAKGQPREPQVYTLPPSRDELTKNQVSLTCLVKGFYP |
| | | | SDIAVEWESNGQPENNYKTTPPVLDSDGSFFLYSRLTVDKSRWQ |
| | | | QGNVFSCSVMHEALHNHYTQKSLSLSPGK |
| | VH 1 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | CH1 | 551 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKKVEPKSCD |
| | Linker | 3308 | GGGGSGGGGS |
| | VH 2 | 523 | QVQLVQSGAEVKKPGSSVKVSCKASGYSFTGYTMNWVRQAPG |
| | | | QGLEWMGLITPYNGASSYNQKFRGRVTITVDKSTSTAYMELSSL |
| | | | RSEDTAVYYCARGGYDGRGFDYWGQGTLVTVSS |
| | HC CDR1 | 524 | GYSFTGYTMN |
| | HC CDR2 | 525 | LITPYNGASSYNQKFRG |
| | HC CDR3 | 526 | GGYDGRGFDY |
| | Heavy | 554 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Knob | | QYASTYRVVSVLTVLHQDWLNGKEYKCKVSNKALPAPIEKTIS |
| | | | KAKGQPREPQVYTLPPSRDELTKNQVSLTCLVKGFYPSDIAVEW |
| | | | ESNGQPENNYKTTPPVLDSDGSFFLYSRLTVDKSRWQQGNVFSC |
| | | | SVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 555 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Knob | | VLHQDWLNGKEYKCKVSNKALPAPIEKTISKAKGQPREPQVYT |
| | | | LPPSRDELTKNQVSLTCLVKGFYPSDIAVEWESNGQPENNYKTT |
| | | | PPVLDSDGSFFLYSRLTVDKSRWQQGNVFSCSVMHEALHNHYT |
| | | | QKSLSLSPGK |
| Polypeptide | Full | 502 | EIVLTQSPATLSLSPGERATLSCSASSSVSYMHWYQQKPGLAPRR |
| Chain 3 | Sequence | | LIYDTSKLASGIPDRFSGSGSGNDYTLTISRLEPEDFAVYYCQQW |
| | | | SKHPLTFGQGTKVEIKRTVAAPSVFIFPPSDEQLKSGTASVVCLL |
| | | | NNFYPREAKVQWKVDNALQSGNSQESVTEQDSKDSTYSLSSTL |
| | | | TLSKADYEKHKVYACEVTHQGLSSPVTKSFNRGEC |
| | VL 1 or 2 | 527 | EIVLTQSPATLSLSPGERATLSCSASSSVSYMHWYQQKPGLAPRR |
| | | | LIYDTSKLASGIPDRFSGSGSGNDYTLTISRLEPEDFAVYYCQQW |
| | | | SKHPLTFGQGTKVEIK |
| | LC CDR1 | 1168 | SASSSVSYMH |
| | LC CDR2 | 1295 | DTSKLAS |
| | LC CDR3 | 528 | QQWSKHPLT |
| | Light Chain | 3644 | RTVAAPSVFIFPPSDEQLKSGTASVVCLLNNFYPREAKVQWKVD |
| | Constant | | NALQSGNSQESVTEQDSKDSTYSLSSTLTLSKADYEKHKVYACE |
| | Region | | VTHQGLSSPVTKSFNRGEC |
| | Human | | |
| | Immuno- | | |
| | globulin | | |
| | kappa | | |
| | constant C | | |
| | region | | |
| Polypeptide | Full | 506 | EVKLVESGGGLVQPGGSLRLSCAASGFTFNTYAMNWVRQAPG |
| Chain 2 | Sequence | | KGLEWVARIRSKYNNYATYYADSVKDRFTISRDDSKSSLYLQM |
| Hole | | | NNLKTEDTAMYYCVRHGNFGNSYVSWFAYWGQGTLVTVSSAS |
| | | | TKGPSVFPLAPSSKSTSGGTAALGCLVKDYFPEPVTVSWNSGAL |
| | | | TSGVHTFPAVLQSSGLYSLSSVVTVPSSSLGTQTYICNVNHKPSN |
| | | | TKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQ |
| | | | YASTYRVVSVLTVLHQDWLNGKEYKCKVSNKALPAPIEKTISK |
| | | | AKGQPREPQVYTLPPSRDELTKNQVSLTCLVKGFYPSDIAVEWE |
| | | | SNGQPENNYKTTPPVLDSDGSFLLYSKLTVDKSRWQQGNVFSCS |
| | | | VMHEALHNHYTQKSLSLSPGK |
| | VH 3 | 529 | EVKLVESGGGLVQPGGSLRLSCAASGFTFNTYAMNWVRQAPG |
| | | | KGLEWVARIRSKYNNYATYYADSVKDRFTISRDDSKSSLYLQM |
| | | | NNLKTEDTAMYYCVRHGNFGNSYVSWFAYWGQGTLVTVSS |
| | HC CDR1 | 530 | GFTFNTYAMN |
| | HC CDR2 | 531 | RIRSKYNNYATYYADSVKD |
| | HC CDR3 | 532 | HGNFGNSYVSWFAY |
| | Heavy | 556 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPELLGGPSVFLFPPKPKDTL |
| | Region | | MISRTPEVTCVVVDVSHEDPEVKFNWYVDGVEVHNAKTKPREE |
| | Hole | | QYASTYRVVSVLTVLHQDWLNGKEYKCKVSNKALPAPIEKTIS |
| | | | KAKGQPREPQVYTLPPSRDELTKNQVSLTCLVKGFYPSDIAVEW |
| | | | ESNGQPENNYKTTPPVLDSDGSFLLYSKLTVDKSRWQQGNVFS |
| | | | CSVMHEALHNHYTQKSLSLSPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 557 | DKTHTCPPCPAPELLGGPSVFLFPPKPKDTLMISRTPEVTCVVVD |
| | CH3) region | | VSHEDPEVKFNWYVDGVEVHNAKTKPREEQYASTYRVVSVLT |
| | Hole | | VLHQDWLNGKEYKCKVSNKALPAPIEKTISKAKGQPREPQVYT |
| | | | LPPSRDELTKNQVSLTCLVKGFYPSDIAVEWESNGQPENNYKTT |
| | | | PPVLDSDGSFLLYSKLTVDKSRWQQGNVFSCSVMHEALHNHYT |
| | | | QKSLSLSPGK |
| Polypeptide | Full | 507 | QAVVTQEPSFSVSPGGTVTLTCRSSTGAVTTSNYANWVQQTPG |
| Chain 4 | Sequence | | QAFRGLIGGTNKRAPGVPARFSGSLIGDKAALTITGAQADDESIY |
| | | | FCALWYSNLWVFGGGTKLTVLGQPKANPTVTLFPPSSEELQAN |
| | | | KATLVCLISDFYPGAVTVAWKADGSPVKAGVETTKPSKQSNNK |
| | | | YAASSYLSLTPEQWKSHRSYSCQVTHEGSTVEKTVAPTECS |
| | VL 3 | 533 | QAVVTQEPSFSVSPGGTVTLTCRSSTGAVTTSNYANWVQQTPG |
| | | | QAFRGLIGGTNKRAPGVPARFSGSLIGDKAALTITGAQADDESIY |
| | | | FCALWYSNLWVFGGGTKLTVL |
| | LC CDR1 | 534 | RSSTGAVTTSNYAN |
| | LC CDR2 | 535 | GTNKRAP |
| | LC CDR3 | 536 | ALWYSNLWV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |

BMM0449

| | | | |
|---|---|---|---|
| Polypeptide | Full | 509 | QVQLVQSGAEVKKPGASVKVSCKASGYTFTGNYINWVRQAPG |
| Chain 1 | Sequence | | QGLEWMGIINPTKGWTLYAQKFQGRVTMTRDTSISTAYMELSS |
| Hole | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDKTHTCPPCPAPNAAGGPSVFIFPPKIKDVLMISLSPIVT |
| | | | CVVVDVSEDDPDVQISWFVNNVEVHTAQTQTHREDYNSTLRVV |
| | | | SALPIQHQDWMSGKEFKCKVNNKDLGAPIERTISKPKGSVRAPQ |
| | | | VCVLPPPEEEMTKKQVTLSCAVTDFMPEDIYVEWTNNGKTELN |
| | | | YKNTEPVLDSDGSYFMVSKLRVEKKNWVERNSYSCSVVHEGL |
| | | | HNHHTTKSFSRTPGK |
| | VH 1 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 559 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Hole | | YNSTLRVVSALPIQHQDWMSGKEFKCKVNNKDLGAPIERTISKP |
| | | | KGSVRAPQVCVLPPPEEEMTKKQVTLSCAVTDFMPEDIYVEWT |
| | | | NNGKTELNYKNTEPVLDSDGSYFMVSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 510 | DIVLTQPPSVSGAPGQRVTISCSGSSSNIGSNYVSWYQQLPGTAP |
| Chain 3 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 1 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |
| Polypeptide | Full | 511 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSG |
| | | | GGGSGGGGSAPTSSSTKKTQLQLEHLLLDLQMILNGINNYKNPK |
| | | | LTRMLTFKFYMPKKATELKHLQCLEEELKPLEEVLNLAQSKNFH |
| | | | LRPRDLISNINVIVLELKGSETTFMCEYADETATIVEFLNRWITFC |
| | | | QSIISTLTGGGGSGGGGSDKTHTCPPCPAPNAAGGPSVFIFPPKIK |
| | | | DVLMISLSPIVTCVVVDVSEDDPDVQISWFVNNVEVHTAQTQTH |
| | | | REDYNSTLRVVSALPIQHQDWMSGKEFKCKVNNKDLGAPIERTI |
| | | | SKPKGSVRAPQVYVLPPCEEEMTKKQVTLWCMVTDFMPEDIYV |
| | | | EWTNNGKTELNYKNTEPVLDSDGSYFMYSKLRVEKKNWVERN |
| | | | SYSCSVVHEGLHNHHTTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 2 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 2 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3309 | GGGGSGGGGSGGGGS |
| | IL-2 | 2191 | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFCQSIISTLT |
| | Linker 3 | 3308 | GGGGSGGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BMM0468

| | | | |
|---|---|---|---|
| Polypeptide | Full | 512 | QVQLVQSGAEVKKPGASVKVSCKASGYTFTGNYINWVRQAPG |
| Chain 1 | Sequence | | QGLEWMGIINPTKGWTLYAQKFQGRVTMTRDTSISTAYMELSS |
| Hole | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDGGGGSGGGGSQVQLVQSGAEVKKPGASVKVSCKAS |
| | | | GYTFTGNYINWVRQAPGQGLEWMGIINPTKGWTLYAQKFQGR |
| | | | VTMTRDTSISTAYMELSSLRSEDTAVYYCARWHHGTWIFDYWG |
| | | | QGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQT |
| | | | YICNVNHKPSNTKVDKRVEPKSCDKTHTCPPCPAPNAAGGPSVF |
| | | | IFPPKIKDVLMISLSPIVTCVVVDVSEDDPDVQISWFVNNVEVHT |
| | | | AQTQTHREDYNSTLRVVSALPIQHQDWMSGKEFKCKVNNKDL |
| | | | GAPIERTISKPKGSVRAPQVCVLPPPEEEMTKKQVTLSCAVTDF |
| | | | MPEDIYVEWTNNGKTELNYKNTEPVLDSDGSYFMVSKLRVEKK |
| | | | NWVERNSYSCSVVHEGLHNHHTTKSFSRTPGK |
| | VH 1 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | CH1 | 548 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSCD |
| | Linker 1 | 3308 | GGGGSGGGGS |
| | VH 2 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 559 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Hole | | YNSTLRVVSALPIQHQDWMSGKEFKCKVNNKDLGAPIERTISKP |
| | | | KGSVRAPQVCVLPPPEEEMTKKQVTLSCAVTDFMPEDIYVEWT |
| | | | NNGKTELNYKNTEPVLDSDGSYFMVSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 510 | DIVLTQPPSVSGAPGQRVTISCSGSSSNIGSNYVSWYQQLPGTAP |
| Chain 3 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 1 or 2 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |
| Polypeptide | Full | 511 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSG |
| | | | GGGSGGGGSAPTSSSTKKTQLQLEHLLLDLQMILNGINNYKNPK |
| | | | LTRMLTFKFYMPKKATELKHLQCLEEELKPLEEVLNLAQSKNFH |
| | | | LRPRDLISNINVIVLELKGSETTFMCEYADETATIVEFLNRWITFC |
| | | | QSIISTLTGGGGSGGGGSDKTHTCPPCPAPNAAGGPSVFIFPPKIK |
| | | | DVLMISLSPIVTCVVVDVSEDDPDVQISWFVNNVEVHTAQTQTH |
| | | | REDYNSTLRVVSALPIQHQDWMSGKEFKCKVNNKDLGAPIERTI |
| | | | SKPKGSVRAPQVYVLPPCEEEMTKKQVTLWCMVTDFMPEDIYV |
| | | | EWTNNGKTELNYKNTEPVLDSDGSYFMYSKLRVEKKNWVERN |
| | | | SYSCSVVHEGLHNHHTTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 3 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 2 | 3801 | GGGGSGGGGSGGGGSGGGGS |
| | VL 3 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 3 | 3309 | GGGGSGGGGSGGGGS |
| | IL-2 | 2191 | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFCQSIISTLT |
| | Linker 4 | 3308 | GGGGSGGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BMM0446

| | | | |
|---|---|---|---|
| Polypeptide | Full | 509 | QVQLVQSGAEVKKPGASVKVSCKASGYTFTGNYINWVRQAPG |
| Chain 1 | Sequence | | QGLEWMGIINPTKGWTLYAQKFQGRVTMTRDTSISTAYMELSS |
| Hole | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDKTHTCPPCPAPNAAGGPSVFIFPPKIKDVLMISLSPIVT |
| | | | CVVVDVSEDDPDVQISWFVNNVEVHTAQTQTHREDYNSTLRVV |
| | | | SALPIQHQDWMSGKEFKCKVNNKDLGAPIERTISKPKGSVRAPQ |
| | | | VCVLPPPEEEMTKKQVTLSCAVTDFMPEDIYVEWTNNGKTELN |
| | | | YKNTEPVLDSDGSYFMVSKLRVEKKNWVERNSYSCSVVHEGL |
| | | | HNHHTTKSFSRTPGK |
| | VH 1 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 559 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Hole | | YNSTLRVVSALPIQHQDWMSGKEFKCKVNNKDLGAPIERTISKP |
| | | | KGSVRAPQVCVLPPPEEEMTKKQVTLSCAVTDFMPEDIYVEWT |
| | | | NNGKTELNYKNTEPVLDSDGSYFMVSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 510 | DIVLTQPPSVSGAPGQRVTISCSGSSSNIGSNYVSWYQQLPGTAP |
| Chain 3 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 1 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |
| Polypeptide | Full | 513 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSD |
| | | | KTHTCPPCPAPNAAGGPSVFIFPPKIKDVLMISLSPIVTCVVVDVS |
| | | | EDDPDVQISWFVNNVEVHTAQTQTHREDYNSTLRVVSALPIQH |
| | | | QDWMSGKEFKCKVNNKDLGAPIERTISKPKGSVRAPQVYVLPP |
| | | | CEEEMTKKQVTLWCMVTDFMPEDIYVEWTNNGKTELNYKNTE |
| | | | PVLDSDGSYFMYSKLRVEKKNWVERNSYSCSVVHEGLHNHHT |
| | | | TKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 2 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 2 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3307 | GGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BNM0096

| | | | |
|---|---|---|---|
| Polypeptide | Full | 512 | QVQLVQSGAEVKKPGASVKVSCKASGYTFTGNYINWVRQAPG |
| Chain 1 | Sequence | | QGLEWMGIINPTKGWTLYAQKFQGRVTMTRDTSISTAYMELSS |
| Hole | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDGGGGSGGGGSQVQLVQSGAEVKKPGASVKVSCKAS |
| | | | GYTFTGNYINWVRQAPGQGLEWMGIINPTKGWTLYAQKFQGR |
| | | | VTMTRDTSISTAYMELSSLRSEDTAVYYCARWHHGTWIFDYWG |
| | | | QGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQT |
| | | | YICNVNHKPSNTKVDKRVEPKSCDKTHTCPPCPAPNAAGGPSVF |
| | | | IFPPKIKDVLMISLSPIVTCVVVDVSEDDPDVQISWFVNNVEVHT |
| | | | AQTQTHREDYNSTLRVVSALPIQHQDWMSGKEFKCKVNNKDL |
| | | | GAPIERTISKPKGSVRAPQVCVLPPPEEEMTKKQVTLSCAVTDF |
| | | | MPEDIYVEWTNNGKTELNYKNTEPVLDSDGSYFMVSKLRVEKK |
| | | | NWVERNSYSCSVVHEGLHNHHTTKSFSRTPGK |
| | VH 1 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | CH1 | 548 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSCD |
| | Linker 1 | 3308 | GGGGSGGGGS |
| | VH 2 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 559 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Hole | | YNSTLRVVSALPIQHQDWMSGKEFKCKVNNKDLGAPIERTISKP |
| | | | KGSVRAPQVCVLPPPEEEMTKKQVTLSCAVTDFMPEDIYVEWT |
| | | | NNGKTELNYKNTEPVLDSDGSYFMVSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 510 | DIVLTQPPSVSGAPGQRVTISCSGSSSNIGSNYVSWYQQLPGTAP |
| Chain 3 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 1 or 2 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |
| Polypeptide | Full | 513 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSD |
| | | | KTHTCPPCPAPNAAGGPSVFIFPPKIKDVLMISLSPIVTCVVVDVS |
| | | | EDDPDVQISWFVNNVEVHTAQTQTHREDYNSTLRVVSALPIQH |
| | | | QDWMSGKEFKCKVNNKDLGAPIERTISKPKGSVRAPQVYVLPP |
| | | | CEEEMTKKQVTLWCMVTDFMPEDIYVEWTNNGKTELNYKNTE |
| | | | PVLDSDGSYFMYSKLRVEKKNWVERNSYSCSVVHEGLHNHHT |
| | | | TKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 3 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 2 | 3801 | GGGGSGGGGSGGGGSGGGGS |
| | VL 3 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 3 | 3307 | GGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BMM0439

| | | | |
|---|---|---|---|
| Polypeptide | Full | 514 | QVQLQQSGTELMKPGASVKISCKASGYTFSNYWIEWIKQRPGH |
| Chain 1 | Sequence | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSQ |
| | | | VQLVQSGAEVKKPGASVKVSCKASGYTFTGNYINWVRQAPGQ |
| | | | GLEWMGIINPTKGWTLYAQKFQGRVTMTRDTSISTAYMELSSL |
| | | | RSEDTAVYYCARWHHGTWIFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKRV |
| | | | EPKSCDKTHTCPPCPAPNAAGGPSVFIFPPKIKDVLMISLSPIVTC |
| | | | VVVDVSEDDPDVQISWFVNNVEVHTAQTQTHREDYNSTLRVVS |
| | | | ALPIQHQDWMSGKEFKCKVNNKDLGAPIERTISKPKGSVRAPQV |
| | | | YVLPPPEEEMTKKQVTLTCMVTDFMPEDIYVEWTNNGKTELNY |
| | | | KNTEPVLDSDGSYFMYSKLRVEKKNWVERNSYSCSVVHEGLH |
| | | | NHHTTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 1 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 1 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3307 | GGGGS |
| | VH 2 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 565 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | | | YNSTLRVVSALPIQHQDWMSGKEFKCKVNNKDLGAPIERTISKP |
| | | | KGSVRAPQVYVLPPPEEEMTKKQVTLTCMVTDFMPEDIYVEWT |
| | | | NNGKTELNYKNTEPVLDSDGSYFMYSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 566 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYFMYSKLRVEKKNWVERNSYSCSVVHEGLHNHHTT |
| | | | KSFSRTPGK |
| Polypeptide | Full | 510 | DIVLTQPPSVSGAPGQRVTISCSGSSSNIGSNYVSWYQQLPGTAP |
| Chain 3 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 2 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |
| Polypeptide | Full | 514 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSQ |
| | | | VQLVQSGAEVKKPGASVKVSCKASGYTFTGNYINWVRQAPGQ |
| | | | GLEWMGIINPTKGWTLYAQKFQGRVTMTRDTSISTAYMELSSL |
| | | | RSEDTAVYYCARWHHGTWIFDYWGQGTLVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKRV |
| | | | EPKSCDKTHTCPPCPAPNAAGGPSVFIFPPKIKDVLMISLSPIVTC |
| | | | VVVDVSEDDPDVQISWFVNNVEVHTAQTQTHREDYNSTLRVVS |
| | | | ALPIQHQDWMSGKEFKCKVNNKDLGAPIERTISKPKGSVRAPQV |
| | | | YVLPPPEEEMTKKQVTLTCMVTDFMPEDIYVEWTNNGKTELNY |
| | | | KNTEPVLDSDGSYFMYSKLRVEKKNWVERNSYSCSVVHEGLH |
| | | | NHHTTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 3 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 3 | 3801 | GGGGSGGGGSGGGGSGGGGS |
| | VL 3 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 4 | 3307 | GGGGS |
| | VH 4 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 565 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGQPSVFIFPPKIKDVL |
| | Region | | MISLSPIVTCVVVDVSEDDPDVQISWFVNNVEVHTAQTQTHRED |
| | | | YNSTLRVVSALPIQHQDWMSGKEFKCKVNNKDLGAPIERTISKP |
| | | | KGSVRAPQVYVLPPPEEEMTKKQVTLTCMVTDFMPEDIYVEWT |
| | | | NNGKTELNYKNTEPVLDSDGSYFMYSKLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 566 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYFMYSKLRVEKKNWVERNSYSCSVVHEGLHNHHTT |
| | | | KSFSRTPGK |
| Polypeptide | Full | 510 | DIVLTQPPSVSGAPGQRVTISCSGSSSNIGSNYVSWYQQLPGTAP |
| Chain 4 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 4 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |

BMM0456

| | | | |
|---|---|---|---|
| Polypeptide | Full | 515 | EVQLVESGGGLVQPGKSLKLSCEASGFTFSGYGMHWVRQAPGR |
| Chain 1 | Sequence | | GLESVAYITSSSINIKYADAVKGRFTVSRDNAKNLLFLQMNILKS |
| Hole | | | EDTAMYYCARFDWDKNYWGQGTMVTVSSAKTTAPSVYPLAP |
| | | | VSGDTTGSSVTLGCLVKGYFPEPVTLTWNSGSLSSGVHTFPAVL |
| | | | QSDLYTLSSSVTVTSSTWPSQSITCNVAHPASSTKVDKKIEPKSC |
| | | | DKTHTCPPCPAPNAAGGPSVFIFPPKIKDVLMISLSPIVTCVVVDV |
| | | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| | VH 1 | 567 | EVQLVESGGGLVQPGKSLKLSCEASGFTFSGYGMHWVRQAPGR |
| | | | GLESVAYITSSSINIKYADAVKGRFTVSRDNAKNLLFLQMNILKS |
| | | | EDTAMYYCARFDWDKNYWGQGTMVTVSS |
| | HC CDR1 | 568 | GFTFSGYGMH |
| | HC CDR2 | 569 | YITSSSINIKYADAVKG |
| | HC CDR3 | 570 | FDWDKNY |
| | Heavy | 571 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPTVTCVVVDVSEDDPDVQISWFVNNVEVHTAQTQTHREDY |
| | Hole | | NSTLRVVSALPIQHQDWMSGKEFKCKVNNKDLGAPIERTISKPK |
| | | | GSVRAPQVYVLPPPEEEMTKKQVTLTCMVTDFMPEDIYVEWTN |
| | | | NGKTELNYKNTEPVLDSDGSYLMYSKLTVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 573 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| Polypeptide | Full | 516 | DIQMTQSPSSLPASLGDRVTINCQASQDISNYLNWYQQKPGKAP |
| Chain 3 | Sequence | | KLLIYYTNKLADGVPSRFSGSGSGRDSSFTISSLESEDIGSYYCQQ |
| | | | YYNYPWTFGPGTKLEIKRADAAPTVSIFPPSSEQLTSGGASVVCF |
| | | | LNNFYPKDINVKWKIDGSERQNGVLNSWTDQDSKDSTYSMSST |
| | | | LTLTKDEYERHNSYTCEATHKTSTSPIVKSFNRNEC |
| | VL 1 | 574 | DIQMTQSPSSLPASLGDRVTINCQASQDISNYLNWYQQKPGKAP |
| | | | KLLIYYTNKLADGVPSRFSGSGSGRDSSFTISSLESEDIGSYYCQQ |
| | | | YYNYPWTFGPGTKLEIK |
| | LC CDR1 | 575 | QASQDISNYLN |
| | LC CDR2 | 576 | YTNKLAD |
| | LC CDR3 | 577 | QQYYNYPWT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 517 | QVQLVQSGAEVKKPGASVKVSCKASGYTFTGNYINWVRQAPG |
| Chain 2 | Sequence | | QGLEWMGIINPTKGWTLYAQKFQGRVTMTRDTSISTAYMELSS |
| Knob | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDKTHTCPPCPAPNAAGGPSVFIFPPKIKDVLMISLSPIVT |
| | | | CVVVDVSEDDPDVQISWFVNNVEVHTAQTQTHREDYNSTLRVV |
| | | | SALPIQHQDWMSGKEFKCKVNNKDLGAPIERTISKPKGSVRAPQ |
| | | | VYVLPPPEEEMTKKQVTLTCMVKDFMPEDIYVEWTNNGKTELN |
| | | | YKNTEPVLDSDGSYFMYSRLRVEKKNWVERNSYSCSVVHEGLH |
| | | | NHHTTKSFSRTPGK |
| | VH 2 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 578 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Knob | | YNSTLRVVSALPIQHQDWMSGKEFKCKVNNKDLGAPIERTISKP |
| | | | KGSVRAPQVYVLPPPEEEMTKKQVTLTCMVKDFMPEDIYVEWT |
| | | | NNGKTELNYKNTEPVLDSDGSYFMYSRLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 579 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPPP |
| | | | EEEMTKKQVTLTCMVKDFMPEDIYVEWTNNGKTELNYKNTEP |
| | | | VLDSDGSYFMYSRLRVEKKNWVERNSYSCSVVHEGLHNHHTT |
| | | | KSFSRTPGK |
| Polypeptide | Full | 510 | DIVLTQPPSVSGAPGQRVTISCSGSSSNIGSNYVSWYQQLPGTAP |
| Chain 4 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 2 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |

BMM0469

| | | | |
|---|---|---|---|
| Polypeptide | Full | 515 | EVQLVESGGGLVQPGKSLKLSCEASGFTFSGYGMHWVRQAPGR |
| Chain 1 | Sequence | | GLESVAYITSSSINIKYADAVKGRFTVSRDNAKNLLFLQMNILKS |
| Hole | | | EDTAMYYCARFDWDKNYWGQGTMVTVSSAKTTAPSVYPLAP |
| | | | VSGDTTGSSVTLGCLVKGYFPEPVTLTWNSGSLSSGVHTFPAVL |
| | | | QSDLYTLSSSVTVTSSTWPSQSITCNVAHPASSTKVDKKIEPKSC |
| | | | DKTHTCPPCPAPNAAGGPSVFIFPPKIKDVLMISLSPIVTCVVVDV |
| | | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| | VH 1 | 567 | EVQLVESGGGLVQPGKSLKLSCEASGFTFSGYGMHWVRQAPGR |
| | | | GLESVAYITSSSINIKYADAVKGRFTVSRDNAKNLLFLQMNILKS |
| | | | EDTAMYYCARFDWDKNYWGQGTMVTVSS |
| | HC CDR1 | 568 | GFTFSGYGMH |
| | HC CDR2 | 569 | YITSSSINIKYADAVKG |
| | HC CDR3 | 570 | FDWDKNY |
| | Heavy | 571 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | Hole | | NSTLRVVSALPIQHQDWMSGKEFKCKVNNKDLGAPIERTISKPK |
| | | | GSVRAPQVYVLPPPEEEMTKKQVTLTCMVTDFMPEDIYVEWTN |
| | | | NGKTELNYKNTEPVLDSDGSYLMYSKLTVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 573 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| Polypeptide | Full | 516 | DIQMTQSPSSLPASLGDRVTINCQASQDISNYLNWYQQKPGKAP |
| Chain 3 | Sequence | | KLLIYYTNKLADGVPSRFSGSGSGRDSSFTISSLESEDIGSYYCQQ |
| | | | YYNYPWTFGPGTKLEIKRADAAPTVSIFPPSSEQLTSGGASVVCF |
| | | | LNNFYPKDINVKWKIDGSERQNGVLNSWTDQDSKDSTYSMSST |
| | | | LTLTKDEYERHNSYTCEATHKTSTSPIVKSFNRNEC |
| | VL 1 | 574 | DIQMTQSPSSLPASLGDRVTINCQASQDISNYLNWYQQKPGKAP |
| | | | KLLIYYTNKLADGVPSRFSGSGSGRDSSFTISSLESEDIGSYYCQQ |
| | | | YYNYPWTFGPGTKLEIK |
| | LC CDR1 | 575 | QASQDISNYLN |
| | LC CDR2 | 576 | YTNKLAD |
| | LC CDR3 | 577 | QQYYNYPWT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 518 | QVQLVQSGAEVKKPGASVKVSCKASGYTFTGNYINWVRQAPG |
| Chain 2 | Sequence | | QGLEWMGIINPTKGWTLYAQKFQGRVTMTRDTSISTAYMELSS |
| Knob | | | LRSEDTAVYYCARWHHGTWIFDYWGQGTLVTVSSASTKGPSVF |
| | | | PLAPSSKSTSGGTAALGCLVKDYFPEPVTVSWNSGALTSGVHTF |
| | | | PAVLQSSGLYSLSSVVTVPSSSLGTQTYICNVNHKPSNTKVDKR |
| | | | VEPKSCDGGGGSGGGGSQVQLVQSGAEVKKPGASVKVSCKAS |
| | | | GYTFTGNYINWVRQAPGQGLEWMGIINPTKGWTLYAQKFQGR |
| | | | VTMTRDTSISTAYMELSSLRSEDTAVYYCARWHHGTWIFDYWG |
| | | | QGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQT |
| | | | YICNVNHKPSNTKVDKRVEPKSCDKTHTCPPCPAPNAAGGPSVF |
| | | | IFPPKIKDVLMISLSPIVTCVVVDVSEDDPDVQISWFVNNVEVHT |
| | | | AQTQTHREDYNSTLRVVSALPIQHQDWMSGKEFKCKVNNKDL |
| | | | GAPIERTISKPKGSVRAPQVYVLPPPEEEMTKKQVTLTCMVKDF |
| | | | MPEDIYVEWTNNGKTELNYKNTEPVLDSDGSYFMYSRLRVEKK |
| | | | NWVERNSYSCSVVHEGLHNHHTTKSFSRTPGK |
| | VH 2 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | CH1 | 548 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSCD |
| | Linker | 3308 | GGGGSGGGGS |
| | VH 3 | 537 | QVQLVQSGAEVKKPGASVKVSCKASGYTFTGNYINWVRQAPG |
| | | | QGLEWMGIINPTKGWTLYAQKFQGRVTMTRDTSISTAYMELSS |
| | | | LRSEDTAVYYCARWHHGTWIFDYWGQGTLVTVSS |
| | HC CDR1 | 538 | GYTFTGNYIN |
| | HC CDR2 | 539 | IINPTKGWTLYAQKFQG |
| | HC CDR3 | 540 | WHHGTWIFDY |
| | Heavy | 578 | ASTKGPSVFPLAPSSKSTSGGTAALGCLVKDYFPEPVTVSWNSG |
| | Chain | | ALTSGVHTFPAVLQSSGLYSLSSVVTVPSSSLGTQTYICNVNHKP |
| | Constant | | SNTKVDKRVEPKSCDKTHTCPPCPAPNAAGGPSVFIFPPKIKDVL |
| | Region | | MISLSPIVTCVVVDVSEDDPDVQISWFVNNVEVHTAQTQTHRED |
| | Knob | | YNSTLRVVSALPIQHQDWMSGKEFKCKVNNKDLGAPIERTISKP |
| | | | KGSVRAPQVYVLPPPEEEMTKKQVTLTCMVKDFMPEDIYVEWT |
| | | | NNGKTELNYKNTEPVLDSDGSYFMYSRLRVEKKNWVERNSYS |
| | | | CSVVHEGLHNHHTTKSFSRTPGK |
| | CH1 | 549 | ASTKGPSVFPLAPSSKSTSGGTAALGCLVKDYFPEPVTVSWNSG |
| | | | ALTSGVHTFPAVLQSSGLYSLSSVVTVPSSSLGTQTYICNVNHKP |
| | | | SNTKVDKRVEPKSC |
| | Fc (CH2- | 579 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPPP |
| | | | EEEMTKKQVTLTCMVKDFMPEDIYVEWTNNGKTELNYKNTEP |
| | | | VLDSDGSYFMYSRLRVEKKNWVERNSYSCSVVHEGLHNHHTT |
| | | | KSFSRTPGK |
| Polypeptide | Full | 510 | DIVLTQPPSVSGAPGQRVTISCSGSSSNIGSNYVSWYQQLPGTAP |
| Chain 4 | Sequence | | KLLIYNDNQRPSGVPDRFSGSKSGTSASLAITGLQSEDEADYYCS |
| | | | TYDRRTFSVFGGGTKLTVLGQPKANPTVTLFPPSSEELQANKAT |
| | | | LVCLISDFYPGAVTVAWKADGSPVKAGVETTKPSKQSNNKYAA |
| | | | SSYLSLTPEQWKSHRSYSCQVTHEGSTVEKTVAPTECS |
| | VL 2 or 3 | 561 | DIVLTQPPSVSGAPGQRVTISCSGSSSNIGSNYVSWYQQLPGTAP |
| | | | KLLIYNDNQRPSGVPDRFSGSKSGTSASLAITGLQSEDEADYYCS |
| | | | TYDRRTFSVFGGGTKLTVL |
| | LC CDR1 | 562 | SGSSSNIGSNYVS |
| | LC CDR2 | 563 | NDNQRPS |
| | LC CDR3 | 564 | STYDRRTFSV |
| | Light Chain | 558 | GQPKANPTVTLFPPSSEELQANKATLVCLISDFYPGAVTVAWKA |
| | Constant | | DGSPVKAGVETTKPSKQSNNKYAASSYLSLTPEQWKSHRSYSC |
| | Region | | QVTHEGSTVEKTVAPTECS |

BNM0094

| | | | |
|---|---|---|---|
| Polypeptide | Full | 519 | EVQLQQSGAELVRPGALVKLSCKTSGFNIKDYFLHWVRQRPDQ |
| Chain 1 | Sequence | | GLEWIGWINPDNGNTVYDPKFQGTASLTADTSSNTVYLQLSGLT |
| Hole | | | SEDTAVYFCTRRDYTYEKAALDYWGQGASVIVSSAKTTAPSVY |
| | | | PLAPVSGDTTGSSVTLGCLVKGYFPEPVTLTWNSGSLSSGVHTFP |
| | | | AVLQSDLYTLSSSVTVTSSTWPSQSITCNVAHPASSTKVDKKIEP |
| | | | KSCDKTHTCPPCPAPNAAGGPSVFIFPPKIKDVLMISLSPIVTCVV |
| | | | VDVSEDDPDVQISWFVNNVEVHTAQTQTHREDYNSTLRVVSAL |
| | | | PIQHQDWMSGKEFKCKVNNKDLGAPIERTISKPKGSVRAPQVCV |
| | | | LPPPEEEMTKKQVTLSCAVTDFMPEDIYVEWTNNGKTELNYKN |
| | | | TEPVLDSDGSYFMVSKLRVEKKNWVERNSYSCSVVHEGLHNH |
| | | | HTTKSFSRTPGK |
| | VH 1 | 580 | EVQLQQSGAELVRPGALVKLSCKTSGFNIKDYFLHWVRQRPDQ |
| | | | GLEWIGWINPDNGNTVYDPKFQGTASLTADTSSNTVYLQLSGLT |
| | | | SEDTAVYFCTRRDYTYEKAALDYWGQGASVIVSS |
| | HC CDR1 | 581 | GFNIKDYFLH |
| | HC CDR2 | 582 | WINPDNGNTVYDPKFQG |
| | HC CDR3 | 583 | RDYTYEKAALDY |
| | Heavy | 584 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | Hole | | NSTLRVVSALPIQHQDWMSGKEFKCKVNNKDLGAPIERTISKPK |
| | | | GSVRAPQVCVLPPPEEEMTKKQVTLSCAVTDFMPEDIYVEWTN |
| | | | NGKTELNYKNTEPVLDSDGSYFMVSKLRVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 520 | AIQMSQSPASLSASVGETVTITCRASGNIYNYLAWYQQKQGKSP |
| Chain 3 | Sequence | | HLLVYDAKTLADGVPSRFSGSGSGTQYSLKISSLQTEDSGNYYC |
| | | | QHFWSLPFTFGSGTKLEIKRADAAPTVSIFPPSSEQLTSGGASVV |
| | | | CFLNNFYPKDINVKWKIDGSERQNGVLNSWTDQDSKDSTYSMS |
| | | | STLTLTKDEYERHNSYTCEATHKTSTSPIVKSFNRNEC |
| | VL 1 | 585 | AIQMSQSPASLSASVGETVTITCRASGNIYNYLAWYQQKQGKSP |
| | | | HLLVYDAKTLADGVPSRFSGSGSGTQYSLKISSLQTEDSGNYYC |
| | | | QHFWSLPFTFGSGTKLEIK |
| | LC CDR1 | 586 | RASGNIYNYLA |
| | LC CDR2 | 587 | DAKTLAD |
| | LC CDR3 | 588 | QHFWSLPFT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 511 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSG |
| | | | GGGSGGGGSAPTSSSTKKTQLQLEHLLLDLQMILNGINNYKNPK |
| | | | LTRMLTFKFYMPKKATELKHLQCLEEELKPLEEVLNLAQSKNFH |
| | | | LRPRDLISNINVIVLELKGSETTFMCEYADETATIVEFLNRWITFC |
| | | | QSIISTLTGGGGSGGGGSDKTHTCPPCPAPNAAGGPSVFIFPPKIK |
| | | | DVLMISLSPIVTCVVVDVSEDDPDVQISWFVNNVEVHTAQTQTH |
| | | | REDYNSTLRVVSALPIQHQDWMSGKEFKCKVNNKDLGAPIERTI |
| | | | SKPKGSVRAPQVYVLPPCEEEMTKKQVTLWCMVTDFMPEDIYV |
| | | | EWTNNGKTELNYKNTEPVLDSDGSYFMYSKLRVEKKNWVERN |
| | | | SYSCSVVHEGLHNHHTTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 2 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 2 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3309 | GGGGSGGGGSGGGGS |
| | IL-2 | 2191 | APTSSSTKKTQLQLEHLLLDLQMILNGINNYKNPKLTRMLTFKF |
| | | | YMPKKATELKHLQCLEEELKPLEEVLNLAQSKNFHLRPRDLISNI |
| | | | NVIVLELKGSETTFMCEYADETATIVEFLNRWITFCQSIISTLT |
| | Linker 3 | 3308 | GGGGSGGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BNM0104

| | | | |
|---|---|---|---|
| Polypeptide | Full | 519 | EVQLQQSGAELVRPGALVKLSCKTSGFNIKDYFLHWVRQRPDQ |
| Chain 1 | Sequence | | GLEWIGWINPDNGNTVYDPKFQGTASLTADTSSNTVYLQLSGLT |
| Hole | | | SEDTAVYFCTRRDYTYEKAALDYWGQGASVIVSSAKTTAPSVY |
| | | | PLAPVSGDTTGSSVTLGCLVKGYFPEPVTLTWNSGSLSSGVHTFP |
| | | | AVLQSDLYTLSSSVTVTSSTWPSQSITCNVAHPASSTKVDKKIEP |
| | | | KSCDKTHTCPPCPAPNAAGGPSVFIFPPKIKDVLMISLSPIVTCVV |
| | | | VDVSEDDPDVQISWFVNNVEVHTAQTQTHREDYNSTLRVVSAL |
| | | | PIQHQDWMSGKEFKCKVNNKDLGAPIERTISKPKGSVRAPQVCV |
| | | | LPPPEEEMTKKQVTLSCAVTDFMPEDIYVEWTNNGKTELNYKN |
| | | | TEPVLDSDGSYFMVSKLRVEKKNWVERNSYSCSVVHEGLHNH |
| | | | HTTKSFSRTPGK |
| | VH 1 | 580 | EVQLQQSGAELVRPGALVKLSCKTSGFNIKDYFLHWVRQRPDQ |
| | | | GLEWIGWINPDNGNTVYDPKFQGTASLTADTSSNTVYLQLSGLT |
| | | | SEDTAVYFCTRRDYTYEKAALDYWGQGASVIVSS |
| | HC CDR1 | 581 | GFNIKDYFLH |
| | HC CDR2 | 582 | WINPDNGNTVYDPKFQG |
| | HC CDR3 | 583 | RDYTYEKAALDY |
| | Heavy | 584 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | Hole | | NSTLRVVSALPIQHQDWMSGKEFKCKVNNKDLGAPIERTISKPK |
| | | | GSVRAPQVCVLPPPEEEMTKKQVTLSCAVTDFMPEDIYVEWTN |
| | | | NGKTELNYKNTEPVLDSDGSYFMVSKLRVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 560 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVCVLPPP |
| | | | EEEMTKKQVTLSCAVTDFMPEDIYVEWTNNGKTELNYKNTEPV |
| | | | LDSDGSYFMVSKLRVEKKNWVERNSYSCSVVHEGLHNHHTTK |
| | | | SFSRTPGK |
| Polypeptide | Full | 520 | AIQMSQSPASLSASVGETVTITCRASGNIYNYLAWYQQKQGKSP |
| Chain 3 | Sequence | | HLLVYDAKTLADGVPSRFSGSGSGTQYSLKISSLQTEDSGNYYC |
| | | | QHFWSLPFTFGSGTKLEIKRADAAPTVSIFPPSSEQLTSGGASVV |
| | | | CFLNNFYPKDINVKWKIDGSERQNGVLNSWTDQDSKDSTYSMS |
| | | | STLTLTKDEYERHNSYTCEATHKTSTSPIVKSFNRNEC |
| | VL 1 | 585 | AIQMSQSPASLSASVGETVTITCRASGNIYNYLAWYQQKQGKSP |
| | | | HLLVYDAKTLADGVPSRFSGSGSGTQYSLKISSLQTEDSGNYYC |
| | | | QHFWSLPFTFGSGTKLEIK |
| | LC CDR1 | 586 | RASGNIYNYLA |
| | LC CDR2 | 587 | DAKTLAD |
| | LC CDR3 | 588 | QHFWSLPFT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 513 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| Knob | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSD |
| | | | KTHTCPPCPAPNAAGGPSVFIFPPKIKDVLMISLSPIVTCVVVDVS |
| | | | EDDPDVQISWFVNNVEVHTAQTQTHREDYNSTLRVVSALPIQH |
| | | | QDWMSGKEFKCKVNNKDLGAPIERTISKPKGSVRAPQVYVLPP |
| | | | CEEEMTKKQVTLWCMVTDFMPEDIYVEWTNNGKTELNYKNTE |
| | | | PVLDSDGSYFMYSKLRVEKKNWVERNSYSCSVVHEGLHNHHT |
| | | | TKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 2 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 2 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3307 | GGGGS |
| | Fc (CH2- | 3533 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPP |
| | mouse Fc | | CEEEMTKKQVTLWCMVTDFMPEDIYVEWTNNGKTELNYKNTE |
| | heavy chain | | PVLDSDGSYFMYSKLRVEKKNWVERNSYSCSVVHEGLHNHHT |
| | comprising | | TKSFSRTPGK |
| | LALAPG | | |
| | mutations | | |
| | (L234A | | |
| | L235A and | | |
| | P329G) | | |

BJM0869

| | | | |
|---|---|---|---|
| Polypeptide | Full | 521 | QVQLQQSGTELMKPGASVKISCKASGYTFSNYWIEWIKQRPGH |
| Chain 1 | Sequence | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSEV |
| | | | QLQQSGAELVRPGALVKLSCKTSGFNIKDYFLHWVRQRPDQGL |
| | | | EWIGWINPDNGNTVYDPKFQGTASLTADTSSNTVYLQLSGLTSE |
| | | | DTAVYFCTRRDYTYEKAALDYWGQGASVIVSSAKTTAPSVYPL |
| | | | APVSGDTTGSSVTLGCLVKGYFPEPVTLTWNSGSLSSGVHTFPA |
| | | | VLQSDLYTLSSSVTVTSSTWPSQSITCNVAHPASSTKVDKKIEPK |
| | | | SCDKTHTCPPCPAPNAAGGPSVFIFPPKIKDVLMISLSPIVTCVVV |
| | | | DVSEDDPDVQISWFVNNVEVHTAQTQTHREDYNSTLRVVSALPI |
| | | | QHQDWMSGKEFKCKVNNKDLGAPIERTISKPKGSVRAPQVYVL |
| | | | PPPEEEMTKKQVTLTCMVTDFMPEDIYVEWTNNGKTELNYKNT |
| | | | EPVLDSDGSYFMYSKLRVEKKNWVERNSYSCSVVHEGLHNHH |
| | | | TTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 1 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 1 | 3801 | GGGGSGGGGSGGGGSGGGGS |
| | VL 1 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 2 | 3307 | GGGGS |
| | VH 2 | 580 | EVQLQQSGAELVRPGALVKLSCKTSGFNIKDYFLHWVRQRPDQ |
| | | | GLEWIGWINPDNGNTVYDPKFQGTASLTADTSSNTVYLQLSGLT |
| | | | SEDTAVYFCTRRDYTYEKAALDYWGQGASVIVSS |
| | HC CDR1 | 581 | GFNIKDYFLH |
| | HC CDR2 | 582 | WINPDNGNTVYDPKFQG |
| | HC CDR3 | 583 | RDYTYEKAALDY |
| | Heavy | 589 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | | | NSTLRVVSALPIQHQDWMSGKEFKCKVNNKDLGAPIERTISKPK |
| | | | GSVRAPQVYVLPPPEEEMTKKQVTLTCMVTDFMPEDIYVEWTN |
| | | | NGKTELNYKNTEPVLDSDGSYFMYSKLRVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 566 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYFMYSKLRVEKKNWVERNSYSCSVVHEGLHNHHTT |
| | | | KSFSRTPGK |
| Polypeptide | Full | 520 | AIQMSQSPASLSASVGETVTITCRASGNIYNYLAWYQQKQGKSP |
| Chain 3 | Sequence | | HLLVYDAKTLADGVPSRFSGSGSGTQYSLKISSLQTEDSGNYYC |
| | | | QHFWSLPFTFGSGTKLEIKRADAAPTVSIFPPSSEQLTSGGASVV |
| | | | CFLNNFYPKDINVKWKIDGSERQNGVLNSWTDQDSKDSTYSMS |
| | | | STLTLTKDEYERHNSYTCEATHKTSTSPIVKSFNRNEC |
| | VL 2 | 585 | AIQMSQSPASLSASVGETVTITCRASGNIYNYLAWYQQKQGKSP |
| | | | HLLVYDAKTLADGVPSRFSGSGSGTQYSLKISSLQTEDSGNYYC |
| | | | QHFWSLPFTFGSGTKLEIK |
| | LC CDR1 | 586 | RASGNIYNYLA |
| | LC CDR2 | 587 | DAKTLAD |
| | LC CDR3 | 588 | QHFWSLPFT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 521 | QVQLQQSGTELMKPGASVKISCKASGYTFSNYWIEWIKQRPGH |
| Chain 2 | Sequence | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIKGGGGSEV |
| | | | QLQQSGAELVRPGALVKLSCKTSGFNIKDYFLHWVRQRPDQGL |
| | | | EWIGWINPDNGNTVYDPKFQGTASLTADTSSNTVYLQLSGLTSE |
| | | | DTAVYFCTRRDYTYEKAALDYWGQGASVIVSSAKTTAPSVYPL |
| | | | APVSGDTTGSSVTLGCLVKGYFPEPVTLTWNSGSLSSGVHTFPA |
| | | | VLQSDLYTLSSSVTVTSSTWPSQSITCNVAHPASSTKVDKKIEPK |
| | | | SCDKTHTCPPCPAPNAAGGPSVFIFPPKIKDVLMISLSPIVTCVVV |
| | | | DVSEDDPDVQISWFVNNVEVHTAQTQTHREDYNSTLRVVSALPI |
| | | | QHQDWMSGKEFKCKVNNKDLGAPIERTISKPKGSVRAPQVYVL |
| | | | PPPEEEMTKKQVTLTCMVTDFMPEDIYVEWTNNGKTELNYKNT |
| | | | EPVLDSDGSYFMYSKLRVEKKNWVERNSYSCSVVHEGLHNHH |
| | | | TTKSFSRTPGK |
| | scFv | 1376 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSAGGGGSGGGG |
| | | | SGGGGSGGGGSDIVMSQSPSSLAVSVGEKVTMSCKSSQSLLYSG |
| | | | NQKNYLAWYQQKPGQSPKLLIYWASTRESGVPDRFTGSGSGTD |
| | | | FTLTINSVKAEDLTVYYCQQYYGYPRTFGGGTKVEIK |
| | VH 3 | 541 | QVQLQQSGTELMKPGASVKISCKASGYTFSNYWIEWIKQRPGH |
| | | | GLEWVGEILPGAGPTNYNEKFKGKATFTADSSSNTAYMQLSSLT |
| | | | SEDSAVYYCARTDYDYDWFAYWGQGTLVTVSA |
| | HC CDR1 | 542 | GYTFSNYWIE |
| | HC CDR2 | 543 | EILPGAGPTNYNEKFKG |
| | HC CDR3 | 544 | TDYDYDWFAY |
| | Linker 3 | 3801 | GGGGSGGGGSGGGGSGGGGS |
| | VL 3 | 3527 | DIVMSQSPSSLAVSVGEKVTMSCKSSQSLLYSGNQKNYLAWYQ |
| | | | QKPGQSPKLLIYWASTRESGVPDRFTGSGSGTDFTLTINSVKAED |
| | | | LTVYYCQQYYGYPRTFGGGTKVEIK |
| | LC CDR1 | 545 | KSSQSLLYSGNQKNYLA |
| | LC CDR2 | 546 | WASTRES |
| | LC CDR3 | 547 | QQYYGYPRT |
| | Linker 4 | 3307 | GGGGS |
| | VH 4 | 580 | EVQLQQSGAELVRPGALVKLSCKTSGFNIKDYFLHWVRQRPDQ |
| | | | GLEWIGWINPDNGNTVYDPKFQGTASLTADTSSNTVYLQLSGLT |
| | | | SEDTAVYFCTRRDYTYEKAALDYWGQGASVIVSS |
| | HC CDR1 | 581 | GFNIKDYFLH |
| | HC CDR2 | 582 | WINPDNGNTVYDPKFQG |
| | HC CDR3 | 583 | RDYTYEKAALDY |
| | Heavy | 589 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | | | NSTLRVVSALPIQHQDWMSGKEFKCKVNNKDLGAPIERTISKPK |
| | | | GSVRAPQVYVLPPPEEEMTKKQVTLTCMVTDFMPEDIYVEWTN |
| | | | NGKTELNYKNTEPVLDSDGSYFMYSKLRVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 566 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYFMYSKLRVEKKNWVERNSYSCSVVHEGLHNHHTT |
| | | | KSFSRTPGK |
| Polypeptide | Full | 520 | AIQMSQSPASLSASVGETVTITCRASGNIYNYLAWYQQKQGKSP |
| Chain 4 | Sequence | | HLLVYDAKTLADGVPSRFSGSGSGTQYSLKISSLQTEDSGNYYC |
| | | | QHFWSLPFTFGSGTKLEIKRADAAPTVSIFPPSSEQLTSGGASVV |
| | | | CFLNNFYPKDINVKWKIDGSERQNGVLNSWTDQDSKDSTYSMS |
| | | | STLTLTKDEYERHNSYTCEATHKTSTSPIVKSFNRNEC |
| | VL 4 | 585 | AIQMSQSPASLSASVGETVTITCRASGNIYNYLAWYQQKQGKSP |
| | | | HLLVYDAKTLADGVPSRFSGSGSGTQYSLKISSLQTEDSGNYYC |
| | | | QHFWSLPFTFGSGTKLEIK |
| | LC CDR1 | 586 | RASGNIYNYLA |
| | LC CDR2 | 587 | DAKTLAD |
| | LC CDR3 | 588 | QHFWSLPFT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |

BNM0092

| | | | |
|---|---|---|---|
| Polypeptide | Full | 515 | EVQLVESGGGLVQPGKSLKLSCEASGFTFSGYGMHWVRQAPGR |
| Chain 1 | Sequence | | GLESVAYITSSSINIKYADAVKGRFTVSRDNAKNLLFLQMNILKS |
| Hole | | | EDTAMYYCARFDWDKNYWGQGTMVTVSSAKTTAPSVYPLAP |
| | | | VSGDTTGSSVTLGCLVKGYFPEPVTLTWNSGSLSSGVHTFPAVL |
| | | | QSDLYTLSSSVTVTSSTWPSQSITCNVAHPASSTKVDKKIEPKSC |
| | | | DKTHTCPPCPAPNAAGGPSVFIFPPKIKDVLMISLSPIVTCVVVDV |
| | | | SEDDPDVQISWFVNNVEVHTAQTQTHREDYNSTLRVVSALPIQH |
| | | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| | VH 1 | 567 | EVQLVESGGGLVQPGKSLKLSCEASGFTFSGYGMHWVRQAPGR |
| | | | GLESVAYITSSSINIKYADAVKGRFTVSRDNAKNLLFLQMNILKS |
| | | | EDTAMYYCARFDWDKNYWGQGTMVTVSS |
| | HC CDR1 | 568 | GFTFSGYGMH |
| | HC CDR2 | 569 | YITSSSINIKYADAVKG |
| | HC CDR3 | 570 | FDWDKNY |
| | Heavy | 571 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | Hole | | NSTLRVVSALPIQHQDWMSGKEFKCKVNNKDLGAPIERTISKPK |
| | | | GSVRAPQVYVLPPPEEEMTKKQVTLTCMVTDFMPEDIYVEWTN |
| | | | NGKTELNYKNTEPVLDSDGSYLMYSKLTVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 573 | DKTHTCPPCPAPNAAGQPSVFIFPPKIKDVLMISLSPIVTCVVVDV |
| | CH3) region | | SEDDPDVQISWFVNNVEVHTAQTQTHREDYNSTLRVVSALPIQH |
| | Hole | | QDWMSGKEFKCKVNNKDLGAPIERTISKPKGSVRAPQVYVLPPP |
| | | | EEEMTKKQVTLTCMVTDFMPEDIYVEWTNNGKTELNYKNTEP |
| | | | VLDSDGSYLMYSKLTVEKKNWVERNSYSCSVVHEGLHNHHTT |
| | | | KSFSRTPGK |
| Polypeptide | Full | 516 | DIQMTQSPSSLPASLGDRVTINCQASQDISNYLNWYQQKPGKAP |
| Chain 3 | Sequence | | KLLIYYTNKLADGVPSRFSGSGSGRDSSFTISSLESEDIGSYYCQQ |
| | | | YYNYPWTFGPGTKLEIKRADAAPTVSIFPPSSEQLTSGGASVVCF |
| | | | LNNFYPKDINVKWKIDGSERQNGVLNSWTDQDSKDSTYSMSST |
| | | | LTLTKDEYERHNSYTCEATHKTSTSPIVKSFNRNEC |
| | VL 1 | 574 | DIQMTQSPSSLPASLGDRVTINCQASQDISNYLNWYQQKPGKAP |
| | | | KLLIYYTNKLADGVPSRFSGSGSGRDSSFTISSLESEDIGSYYCQQ |
| | | | YYNYPWTFGPGTKLEIK |
| | LC CDR1 | 575 | QASQDISNYLN |
| | LC CDR2 | 576 | YTNKLAD |
| | LC CDR3 | 577 | QQYYNYPWT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |
| Polypeptide | Full | 522 | EVQLQQSGAELVRPGALVKLSCKTSGFNIKDYFLHWVRQRPDQ |
| Chain 2 | Sequence | | GLEWIGWINPDNGNTVYDPKFQGTASLTADTSSNTVYLQLSGLT |
| Knob | | | SEDTAVYFCTRRDYTYEKAALDYWGQGASVIVSSAKTTAPSVY |
| | | | PLAPVSGDTTGSSVTLGCLVKGYFPEPVTLTWNSGSLSSGVHTFP |
| | | | AVLQSDLYTLSSSVTVTSSTWPSQSITCNVAHPASSTKVDKKIEP |
| | | | KSCDKTHTCPPCPAPNAAGGPSVFIFPPKIKDVLMISLSPIVTCVV |
| | | | VDVSEDDPDVQISWFVNNVEVHTAQTQTHREDYNSTLRVVSAL |
| | | | PIQHQDWMSGKEFKCKVNNKDLGAPIERTISKPKGSVRAPQVY |
| | | | VLPPPEEEMTKKQVTLTCMVKDFMPEDIYVEWTNNGKTELNYK |
| | | | NTEPVLDSDGSYFMYSRLRVEKKNWVERNSYSCSVVHEGLHNH |
| | | | HTTKSFSRTPGK |
| | VH 2 | 580 | EVQLQQSGAELVRPGALVKLSCKTSGFNIKDYFLHWVRQRPDQ |
| | | | GLEWIGWINPDNGNTVYDPKFQGTASLTADTSSNTVYLQLSGLT |
| | | | SEDTAVYFCTRRDYTYEKAALDYWGQGASVIVSS |
| | HC CDR1 | 581 | GFNIKDYFLH |
| | HC CDR2 | 582 | WINPDNGNTVYDPKFQG |
| | HC CDR3 | 583 | RDYTYEKAALDY |
| | Heavy | 3802 | AKTTAPSVYPLAPVSGDTTGSSVTLGCLVKGYFPEPVTLTWNSG |
| | Chain | | SLSSGVHTFPAVLQSDLYTLSSSVTVTSSTWPSQSITCNVAHPAS |
| | Constant | | STKVDKKIEPKSCDKTHTCPPCPAPNAAGGPSVFIFPPKIKDVLMI |
| | Region | | SLSPIVTCVVVDVSEDDPDVQISWFVNNVEVHTAQTQTHREDY |
| | Knob | | NSTLRVVSALPIQHQDWMSGKEFKCKVNNKDLGAPIERTISKPK |
| | | | GSVRAPQVYVLPPPEEEMTKKQVTLTCMVKDFMPEDIYVEWTN |
| | | | NGKTELNYKNTEPVLDSDGSYFMYSRLRVEKKNWVERNSYSCS |
| | | | VVHEGLHNHHTTKSFSRTPGK |
| | CH1 | 572 | AKTTAPSVYPLAPVSGDTTGSSVTLGCLVKGYFPEPVTLTWNSG |
| | | | SLSSGVHTFPAVLQSDLYTLSSSVTVTSSTWPSQSITCNVAHPAS |
| | | | STKVDKKIEPKSC |
| | Fc (CH2- | 579 | DKTHTCPPCPAPNAAGGPSVFIFPPKIKDVLMISLSPIVTCVVVDV |
| | CH3) region | | SEDDPDVQISWFVNNVEVHTAQTQTHREDYNSTLRVVSALPIQH |
| | Knob | | QDWMSGKEFKCKVNNKDLGAPIERTISKPKGSVRAPQVYVLPPP |
| | | | EEEMTKKQVTLTCMVKDFMPEDIYVEWTNNGKTELNYKNTEP |
| | | | VLDSDGSYFMYSRLRVEKKNWVERNSYSCSVVHEGLHNHHTT |
| | | | KSFSRTPGK |
| Polypeptide | Full | 520 | AIQMSQSPASLSASVGETVTITCRASGNIYNYLAWYQQKQGKSP |
| Chain 4 | Sequence | | HLLVYDAKTLADGVPSRFSGSGSGTQYSLKISSLQTEDSGNYYC |
| | | | QHFWSLPFTFGSGTKLEIKRADAAPTVSIFPPSSEQLTSGGASVV |
| | | | CFLNNFYPKDINVKWKIDGSERQNGVLNSWTDQDSKDSTYSMS |
| | | | STLTLTKDEYERHNSYTCEATHKTSTSPIVKSFNRNEC |
| | VL 2 | 585 | AIQMSQSPASLSASVGETVTITCRASGNIYNYLAWYQQKQGKSP |
| | | | HLLVYDAKTLADGVPSRFSGSGSGTQYSLKISSLQTEDSGNYYC |
| | | | QHFWSLPFTFGSGTKLEIK |
| | LC CDR1 | 586 | RASGNIYNYLA |
| | LC CDR2 | 587 | DAKTLAD |
| | LC CDR3 | 588 | QHFWSLPFT |
| | Light Chain | 3528 | RADAAPTVSIFPPSSEQLTSGGASVVCFLNNFYPKDINVKWKIDG |
| | Constant | | SERQNGVLNSWTDQDSKDSTYSMSSTLTLTKDEYERHNSYTCE |
| | Region | | ATHKTSTSPIVKSFNRNEC |

### Exemplary Embodiments

In some embodiments, binding of the multifunctional polypeptide molecule as described herein to the TCRβV region results in a cytokine profile that differs from a cytokine profile of a T cell engager that binds to a receptor or molecule other than a TCRβV region (non-TCRβV-binding T cell engager). IN some embodiments, the non-TCRβV-binding T cell engager comprises an antibody that binds to a CD3 molecule (e.g., CD3 epsilon (CD3e) molecule); or a TCR alpha (TCRa) molecule.

In some embodiments, the multifunctional polypeptide molecule as described herein further comprises one or more of: a tumor-targeting moiety, a cytokine molecule, a stromal modifying moiety, or an anti-TCRβV antibody molecule other than the first moiety.

In some embodiments, the cytokine profile resulted in by binding of the multifunctional polypeptide molecule as described herein to the TCRβV region comprises, one, two, three, four, five, six, seven, or all of the following: (i) increased level, e.g., expression level, and/or activity of IL-2; (ii) reduced level, e.g., expression level, and/or activity of IL-1β; (iii) reduced level, e.g., expression level, and/or activity of IL-6; (iv) reduced level, e.g., expression level, and/or activity of TNFα; (v) reduced level, e.g., expression level, and/or activity of IL-10; (vi) a delay, e.g., at least 1, 2, 3, 4, 5, 6, 7, 8, 9, 10 or more hours delay, in increased level, e.g., expression level, and/or activity of IL-2; (vii) a delay, e.g., at least 1, 2, 3, 4, 5, 6, 7, 8, 9, 10 hours delay, in increased level, e.g., expression level, and/or activity of IFNg; or (viii) increased level, e.g., expression level, and/or activity of IL-15, e.g., wherein (i)-(viii) are relative to the cytokine profile of the non-TCRβV-binding T cell engager.

In some embodiments, binding of the multifunctional polypeptide molecule as described herein to the TCRβV region results in reduced cytokine storm, e.g., reduced cytokine release syndrome (CRS), as measured by an assay of Example 3, e.g., relative to the cytokine storm induced by the non-TCRβV-binding T cell engager.

In some embodiments, binding of the multifunctional polypeptide molecule as described herein to the TCRβV region results in one, two, three or all of: (i) reduced T cell proliferation kinetics; (ii) cell killing, e.g., target cell killing, e.g. cancer cell killing, e.g., as measured by an assay of Example 4; (iii) increased Natural Killer (NK) cell proliferation, e.g., expansion; or (vi) expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., wherein (ix)-(xii) are relative to the non-TCRβV-binding T cell engager. In some embodiments, the population of T cells having a memory-like phenotype comprises CD45RA+ CCR7− T cells, e.g., CD4+ and/or CD8+ T cells.

In some embodiments, the multifunctional polypeptide molecule as described herein binds to one or more of a TCRβV subfamily chosen from: (i) TCRβ V6 subfamily comprising, e.g., one or more of TCRB V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01; (ii) TCRβ V10 subfamily comprising, e.g., one or more of TCRβ V10-1*01, TCRβ V10-1*02, TCRβ V10-3*01 or TCRβ V10-2*01; (iii) TCRβ V5 subfamily comprising, e.g., one or more of TCRβ V5-6*01, TCRβ V5-4*01, TCRβ V5-1*01 or TCRβ V5-8*01; (iv) TCRβ V12 subfamily comprising, e.g., one or more of TCRβ V12-4*01, TCRβ V12-3*01, or TCRβ V12-5*01; (v) TCRβ V27 subfamily; (vi) TCRβ V28 subfamily; (vii) TCRβ V4 subfamily comprising, e.g., one or more of TCRβ V4-1, TCRβ V4-2 or TCRβ V4-3; (viii) TCRβ V19 subfamily; (ix) TCRβ V9 subfamily; or (x) TCRβ V11 subfamily comprising, e.g., TCRβ V11-2.

In some embodiments, the multifunctional polypeptide molecule as described herein comprises the anti-TCRβV antibody molecule: (i) binds specifically to an epitope on TCRβV, e.g., the same or similar epitope as the epitope recognized by an anti-TCRβV antibody molecule as described herein, e.g., a second anti-TCRβV antibody molecule; (ii) shows the same or similar binding affinity or specificity, or both, as an anti-TCRβV antibody molecule as described herein, e.g., a second anti-TCRβV antibody molecule; (iii) inhibits, e.g., competitively inhibits, the binding of an anti-TCRβV antibody molecule as described herein, e.g., a second anti-TCRβV antibody molecule; or (iv) binds the same or an overlapping epitope with an anti-TCRβV antibody molecule as described herein, e.g., a second anti-TCRβV antibody molecule.

In some embodiments, the multifunctional polypeptide molecule as described herein comprises an antibody molecule chosen from a bispecific antibody molecule, a bivalent antibody molecule, or a biparatopic antibody molecule.

In some embodiments, the multifunctional polypeptide molecule as described herein comprises a bispecific antibody molecule that binds to two different TCRβV subfamily members.

In some embodiments, the multifunctional polypeptide molecule as described herein comprises an anti-TCRβV antibody molecule comprising a heavy chain comprising a framework region, e.g., framework region 3 (FR3), comprising one or both of: (i) a Threonine at position 73, e.g., a substitution at position 73 according to Kabat numbering, e.g., a Glutamic Acid to Threonine substitution; or (ii) a Glycine a position 94. e.g., a substitution at position 94 according to Kabat numbering, e.g., a Arginine to Glycine substitution; wherein the substitution is relative to a human germline heavy chain framework region sequence.

In some embodiments, the multifunctional polypeptide molecule as described herein comprises an anti-TCRβV antibody molecule comprises a light chain comprising a framework region, e.g., framework region 1 (FR1), comprising a Phenylalanine at position 10, e.g., a substitution at position 10 according to Kabat numbering, e.g., a Serine to Phenylalanine substitution, wherein the substitution is relative to a human germline light chain framework region sequence.

In some embodiments, the multifunctional polypeptide molecule as described herein comprises an anti-TCRβV antibody molecule comprises a light chain comprising a framework region, e.g., framework region 2 (FR2), comprising one or both of (i) a Histidine at position 36, e.g., a substitution at position 36 according to Kabat numbering, e.g., a Tyrosine to Histidine substitution; or (ii) an Alanine at position 46, e.g., a substitution at position 46 according to Kabat numbering, e.g., a Arginine to Alanine substitution; wherein the substitution is relative to a human germline light chain framework region sequence.

In some embodiments, the multifunctional polypeptide molecule as described herein comprises an anti-TCRβV antibody molecule comprises a light chain comprising a framework region, e.g., framework region 3 (FR3), comprising a Phenylalanine at position 87, e.g., a substitution at position 87 according to Kabat numbering, e.g., a Tyrosine to Phenylalanine substitution, wherein the substitution is relative to a human germline light chain framework region sequence.

In some embodiments, the multifunctional polypeptide molecule as described herein comprises an anti-TCRβV antibody molecule comprises an antigen binding domain comprising: (a) a light chain variable region (VL) comprising: (i) one, two or all of (e.g., three) a light chain complementarity determining region 1 (LC CDR1), a light chain complementarity determining region 2 (LC CDR2), and a light chain complementarity determining region 3 (LC CDR3) of a humanized B-H light chain (LC) of Table 2; and (ii) a framework region (FR) having at least 95% sequence identity with one, two, three or all (e.g., four) of a framework region 1 (FR1), a framework region 2 (FR2), a framework region 3 (FR3), and a framework region 4 (FR4) of a humanized B-H LC of Table 2; and/or (b) a heavy chain variable region (VH) comprising: (i) one, two or all of (e.g., three) a heavy chain complementarity determining region 1 (HC CDR1), a heavy chain complementarity determining region 2 (HC CDR2) and a heavy chain complementarity determining region 3 (HC CDR3) of a humanized B-H heavy chain (HC) of Table 2; and (ii) a framework region (FR) having at least 95% sequence identity with one, two, three or all (e.g., four) of a framework region 1 (FR1), a framework region 2 (FR2), a framework region 3 (FR3), and a framework region 4 (FR4) of a humanized B-H HC of Table 2.

In some embodiments, the multifunctional polypeptide molecule as described herein comprises an anti-TCRβV antibody molecule comprising an antigen binding domain comprising: (i) a HC CDR1, a HC CDR2 and a HC CDR3 of Antibody B-H listed in Table 2; or (ii) a LC CDR1, a LC CDR2, and a LC CDR3 of Antibody B-H listed in Table 2.

In some embodiments, the multifunctional polypeptide molecule as described herein comprises an anti-TCRβV antibody molecule comprising an antigen binding domain comprising a heavy chain variable region (VH) comprising one, two or all (e.g., three) of a HC CDR1, a HC CDR2 and a HC CDR3 of a humanized Antibody B-H listed in Table 2.

In some embodiments, the multifunctional polypeptide molecule as described herein comprises an anti-TCRβV antibody molecule comprising an antigen binding domain comprising a light chain variable region (VL) comprising one, two or all (e.g., three) of a LC CDR1, a LC CDR2 and a LC CDR3 of a humanized Antibody B-H listed in Table 2.

In some embodiments, the multifunctional polypeptide molecule as described herein comprises an anti-TCRβV antibody molecule comprising: a VH sequence of a humanized Antibody B-H listed in Table 2, or a sequence having at least about 75%, 80%, 85%, 90%, 95%, 99%, 99.5%, 99.7%, 99.9%, or 100% sequence identity to a VH of a humanized Antibody B-H listed in Table 2; and/or a VL sequence of a humanized Antibody B-H listed in Table 2, or a sequence having at least about 75%, 80%, 85%, 90%, 95%, 99%, 99.5%, 99.7%, 99.9%, or 100% sequence identity to a VL of a humanized Antibody B-H listed in Table 2.

In some embodiments, the multifunctional polypeptide molecule as described herein comprises an anti-TCRβV antibody molecule comprising a framework region (FR) having at least 75%, 80%, 85%, 90%, 95%, 99%, 99.5%, 99.7%, 99.9%, or 100% sequence identity with one of: a FR1, a FR2, a FR3, and a FR4 of a humanized B-H LC of Table 2.

In some embodiments, the multifunctional polypeptide molecule as described herein comprises an anti-TCRβV antibody molecule comprising a framework region (FR) having at least 75%, 80%, 85%, 90%, 95%, 99%, 99.5%, 99.7%, 99.9%, or 100% sequence identity with any two of: a FR1, a FR2, a FR3, and a FR4 of a humanized B-H LC of Table 2.

In some embodiments, the multifunctional polypeptide molecule as described herein comprises an anti-TCRβV antibody molecule comprising a framework region (FR) having at least 75%, 80%, 85%, 90%, 95%, 99%, 99.5%, 99.7%, 99.9%, or 100% sequence identity with any three of: a FR1, a FR2, a FR3, and a FR4 of a humanized B-H LC of Table 2.

In some embodiments, the multifunctional polypeptide molecule as described herein comprises an anti-TCRβV antibody molecule comprises a framework region (FR) having at least 75%, 80%, 85%, 90%, 95%, 99%, 99.5%, 99.7%, 99.9%, or 100% sequence identity with all of, a FR1, a FR2, a FR3, and a FR4 of a humanized B-H LC of Table 2.

In some embodiments, the multifunctional polypeptide molecule as described herein comprises an anti-TCRβV antibody molecule comprises a framework region (FR) having at least 75%, 80%, 85%, 90%, 95%, 99%, 99.5%, 99.7%, 99.9%, or 100% sequence identity with one of: a FR1, a FR2, a FR3, and a FR4 of a humanized B-H HC of Table 2.

In some embodiments, the multifunctional polypeptide molecule as described herein comprises an anti-TCRβV antibody molecule comprises a framework region (FR) having at least 75%, 80%, 85%, 90%, 95%, 99%, 99.5%, 99.7%, 99.9%, or 100% sequence identity with any two of: a FR1, a FR2, a FR3, and a FR4 of a humanized B-H HC of Table 2.

In some embodiments, the multifunctional polypeptide molecule as described herein comprises an anti-TCRβV antibody molecule comprises a framework region (FR) having at least 75%, 80%, 85%, 90%, 95%, 99%, 99.5%, 99.7%, 99.9%, or 100% sequence identity with any three of: a FR1, a FR2, a FR3, and a FR4 of a humanized B-H HC of Table 2.

In some embodiments, the multifunctional polypeptide molecule as described herein comprises an anti-TCRβV antibody molecule comprises a framework region (FR) having at least 75%, 80%, 85%, 90%, 95%, 99%, 99.5%, 99.7%, 99.9%, or 100% sequence identity with all of: a FR1, a FR2, a FR3, and a FR4 of a humanized B-H HC of Table 2.

In some embodiments, binding of the multifunctional polypeptide molecule as described herein to a TCRβV region results in a reduction of at least 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 fold, or at least 2-10000 fold in the expression level and or activity of IL-1β as measured by an assay of Example 3.

In some embodiments, binding of the multifunctional polypeptide molecule as described herein to a TCRβV region results in a reduction of at least 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 fold, or at least 2-10000 fold in the expression level and or activity of IL-6 as measured by an assay of Example 3.

In some embodiments, binding of the multifunctional polypeptide molecule as described herein to a TCRβV region results in a reduction of at least 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 fold, or at least 2-10000 fold in the expression level and or activity of TNFα as measured by an assay of Example 3.

In some embodiments, binding of the multifunctional polypeptide molecule as described herein to a TCRβV region results in a reduction of at least 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 fold, or at least 2-10000 fold in the expression level and or activity of IL-2 as measured by an assay of Example 3.

In some embodiments, the multifunctional polypeptide molecule as described herein comprises an anti-TCRβV antibody molecule that binds to a conformational or a linear epitope on the T cell.

In some embodiments, the multifunctional polypeptide molecule as described herein comprises an anti-TCRβV antibody molecule that is a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the multifunctional polypeptide molecule as described herein comprises an anti-TCRβV antibody molecule that comprises one or more heavy chain constant regions chosen from IgG1, IgG2, IgG3, IgGA1, IgGA2, IgG4, IgJ, IgM, IgD, or IgE, or a fragment thereof, e.g., as described in Table 3, 21, or 22.

In some embodiments, the multifunctional polypeptide molecule as described herein comprises an anti-TCRβV antibody molecule that comprises a heavy chain constant region of an IgM or a fragment thereof, optionally wherein the IgM heavy chain constant region comprises the sequence of SEQ ID NO: 73, or a sequence with at least 75%, 80%, 85%, 90%, 95%, 99%, 99.5%, 99.7%, 99.9%, or 100% sequence identity thereto.

In some embodiments, the multifunctional polypeptide molecule as described herein comprises an anti-TCRβV antibody molecule that comprises a heavy chain constant region of an IgJ or a fragment thereof, optionally wherein the IgJ heavy chain constant region comprises the sequence of SEQ ID NO: 76 or a sequence with at least 75%, 80%, 85%, 90%, 95%, 99%, 99.5%, 99.7%, 99.9%, or 100% sequence identity thereto.

In some embodiments, the multifunctional polypeptide molecule as described herein comprises an anti-TCRβV antibody molecule that comprises a heavy chain constant region of an IgGA1, or a fragment thereof, optionally wherein the IgGA1 heavy chain constant region comprises the sequence of SEQ ID NO: 74, or a sequence at least 75%, 80%, 85%, 90%, 95%, 99%, 99.5%, 99.7%, 99.9%, or 100% sequence identity thereto.

In some embodiments, the multifunctional polypeptide molecule as described herein comprises an anti-TCRβV antibody molecule that comprises a heavy chain constant region of an IgGA2, or a fragment thereof, optionally wherein the IgGA2 heavy chain constant region comprises a sequence listed in Table 3, 21, or 22, e.g., SEQ ID NO: 75, or a sequence with at least 75%, 80%, 85%, 90%, 95%, 99%, 99.5%, 99.7%, 99.9%, or 100% sequence identity thereto.

In some embodiments, the multifunctional polypeptide molecule as described herein comprises an anti-TCRβV antibody molecule that comprises a light chain constant region chosen from the light chain constant regions of kappa or lambda, or a fragment thereof, e.g., as described in Table 3, 21, or 22.

In some embodiments, the multifunctional polypeptide molecule as described herein comprises an anti-TCRβV antibody molecule that comprises a light chain constant region of a kappa chain, or a fragment thereof, optionally wherein the kappa chain constant region comprises the sequence of SEQ ID NO: 39, or a sequence with at least 75%, 80%, 85%, 90%, 95%, 99%, 99.5%, 99.7%, 99.9%, or 100% sequence identity thereto.

In some embodiments, the multifunctional polypeptide molecule as described herein comprises an anti-TCRβV antibody molecule that comprises: (i) one or more heavy chain constant regions comprising a heavy chain constant region chosen from IgG1. IgG2, IgG3, IgGA1, IgGA2, IgG4, IgJ, IgM, IgD, or IgE, or a fragment thereof, e.g., as described in Table 3, 21, or 22; and (ii) a light chain constant region comprising a light chain constant region chosen from the light chain constant regions of kappa or lambda, or a fragment thereof, e.g., as described in Table 3, 21, or 22.

In some embodiments, the multifunctional polypeptide molecule as described herein binds to and activates an immune cell, e.g., an effector cell.

In some embodiments, the multifunctional polypeptide molecule as described herein binds to, but does not activate an immune cell, e.g., an effector cell.

In some embodiments, the multifunctional polypeptide molecule as described herein further comprises an NK cell engager, a T cell engager other than an anti-TCRβV antibody molecule, a B cell engager, a dendritic cell engager, or a macrophage cell engager, or a combination thereof.

In some embodiments, the multifunctional polypeptide molecule as described herein further comprises a tumor-targeting moiety that binds to a cancer antigen present on a cancer, e.g., a hematological cancer, a solid tumor, a metastatic cancer, soft tissue tumor, metastatic lesion, or a combination thereof.

In some embodiments, the cancer antigen is a tumor antigen or stromal antigen, or a hematological antigen.

In some embodiments, the cancer antigen is selected from: BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu. Telomerase, SAP-1, Survivin, NY-ESO-1/LAGE-1, PRAME. SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-J, CA-125, BAGE, GAGE, NY-ESO-1, β-catenin, CDK4, CDC27, a actinin-4, TRP 1/gp75, TRP2, gp100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Integrins (Integrin alphaVbeta3, Integrin alpha5Beta1). Carbohydrates (Le), IGF1R, EPHA3, TRAILR1, TRAILR2, RANKL, (FAP), TGF-beta, hyaluronic acid, collagen, e.g., collagen IV, tenascin C, and tenascin W. \

In some embodiments, the tumor-associated antigen binding moiety is a BCMA targeting moiety or a FcRH5 targeting moiety.

In some embodiments, the cancer is a solid tumor including but not limited to: pancreatic (e.g., pancreatic adenocarcinoma) cancer, breast cancer, colorectal cancer, lung cancer (e.g., small or non-small cell lung cancer), skin cancer, ovarian cancer, or liver cancer.

In some embodiments, the cancer is a hematological cancer including, but not limited to: a B-cell or T cell malignancy, e.g., Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome, multiple myeloma, and acute lymphocytic leukemia.

In some embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment, variant or a combination thereof.

In some embodiments, the cytokine molecule is a monomer or a dimer.

In some embodiments, the cytokine molecule further comprises a receptor dimerizing domain, e.g., an IL15Ralpha dimerizing domain.

In some embodiments, the cytokine molecule (e.g., IL-15) and the receptor dimerizing domain (e.g., an IL15Ralpha dimerizing domain) are not covalently linked, e.g., are non-covalently associated.

In some embodiments, the multifunctional polypeptide molecule as described herein further comprises an immunoglobulin constant region (e.g., Fc region) chosen from the heavy chain constant regions of IgG1. IgG2, IgG3, IgGA1. IgGA2. IgG4, IgJ, IgM. IgD, or lgE, or a fragment thereof, optionally wherein, the heavy chain constant region comprises the heavy chain constant region of human IgG1, IgG2 or IgG4.

In some embodiments, the immunoglobulin constant region (e.g., an Fc region) is linked, e.g., covalently linked to, one or more of tumor-targeting moiety, the cytokine molecule, or the stromal modifying moiety.

In some embodiments, an interface of a first and second immunoglobulin chain constant regions (e.g., Fc region) is altered, e.g., mutated, to increase or decrease dimerization, e.g., relative to a non-engineered interface.

In some embodiments, the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

In some embodiments, the multifunctional polypeptide molecule as described herein further comprises a linker, e.g., a linker described herein, optionally wherein the linker is selected from: a cleavable linker, a non-cleavable linker, a peptide linker, a flexible linker, a rigid linker, a helical linker, or a non-helical linker.

In some embodiments, provided herein is an isolated nucleic acid molecule comprising a nucleotide sequence encoding the multifunctional polypeptide molecule as described herein, or a nucleotide sequence having at least 75%, 80%, 85%, 90%, 95%, 99%, 99.5%, 99.7%, 99.9%, or 100% sequence identity thereto.

In some embodiments, provided herein is a vector, e.g., an expression vector, comprising one or more of the nucleic acid molecules as described herein.

In some embodiments, provided herein is a cell, e.g., host cell, comprising the nucleic acid molecule as described herein or the vector of as described herein.

In some embodiments, provided herein is a method of making, e.g., producing or manufacturing, the multifunctional polypeptide molecule as described herein, comprising culturing the host cell as described herein, under suitable conditions, e.g., conditions suitable expression of the multifunctional polypeptide molecule as described herein.

In some embodiments, provided herein is a pharmaceutical composition comprising the multifunctional polypeptide molecule as described herein, and a pharmaceutically acceptable carrier, excipient, or diluent.

In some embodiments, provided herein is a method of modulating, e.g., enhancing, an immune response in a subject comprising administering to the subject an effective amount of the multifunctional polypeptide molecule as described herein. In some embodiments, the method comprises expanding, e.g., increasing the number of, an immune cell population in the subject.

In some embodiments, provided herein is a method of expanding, e.g, increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, the immune cell population comprises a TCRβV expressing cell, e.g., a TCRβV+ cell.

In some embodiments, the TCRβV expressing cell is a T cell, e.g., a CD8+ T cell, a CD3+ T cell or a CD4+ T cell.

In some embodiments, the immune cell population comprises a T cell (e.g., a CD4 T cell, a CD8 T cell (e.g., an effector T cell, a T cell having a memory-like phenotype or a memory T cell (e.g., a memory effector T cell (e.g., TEM cell, e.g., TEMRA cell), or a tumor infiltrating lymphocyte (TIL).

In some embodiments, the immune cell population comprises a T cell, a Natural Killer cell, a B cell, or a myeloid cell.

In some embodiments, the immune cell population is obtained from a healthy subject.

In some embodiments, the immune cell population is obtained from a subject (e.g., from an apheresis sample from the subject) having a disease, e.g., a cancer, e.g., as described herein, optionally wherein the immune cell population comprises a tumor infiltrating lymphocyte (TIL).

In some embodiments, the method results in an expansion of at least 1.1-10 fold (e.g., at least 1.1, 1.2, 1.3, 1.4, 1.5, 2, 3, 4, 5, 6, 7, 8, 9, or 10 fold expansion).

In some embodiments, the method further comprises contacting the population of cells with an agent that promotes, e.g., increases, immune cell expansion.

In some embodiments, the method further comprises contacting the population of cells with an immune checkpoint inhibitor, e.g., a PD-1 inhibitor.

In some embodiments, the method further comprises contacting the population of cells with a 4-1BB (CD 127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, the population of cells is expanded in an appropriate media (e.g., media described herein) that includes one or more cytokines, e.g., IL-2, IL-7, IL-15, or a combination thereof.

In some embodiments, the population of cells is expanded for a period of at least about 4 hours, 6 hours, 10 hours. 12 hours, 15 hours, 18 hours, 20 hours, or 22 hours, or for at least 1, 2, 3, 4, 5, 6, 7, 8, 9, 10, 11, 12, 13, 14, 15, 16, 17, 18, 19, 20 or 21 days, or for at least about 1 week, 2 weeks, 3 weeks, 4 weeks, 5 weeks, 6 weeks, 7 weeks or 8 weeks.

In some embodiments, expansion of the population of immune cells, is compared to expansion of a similar population of cells with an antibody that binds to: a CD3 molecule, e.g., CD3 epsilon (CD3e) molecule; or a TCR alpha (TCRa) molecule. In some embodiments, expansion of the population of immune cells, is compared to expansion of a similar population of cells not contacted with the anti-TCRβV antibody molecule, or multispecific molecule comprising the anti-TCRβV antibody molecule.

In some embodiments, expansion of the population of T cells having a memory-like phenotype, e.g., CD45RA+ CCR7− cells (e.g., memory effector T cells, e.g., TEM cells, e.g., TEMRA cells), is compared to expansion of a similar population of cells with an antibody that binds to: a CD3 molecule, e.g., CD3 epsilon (CD3e) molecule; or a TCR alpha (TCRa) molecule.

In some embodiments, the population of expanded T cells having a memory-like phenotype, e.g., effector memory cells, comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7-, CD95+ T cells, optionally wherein the T cells comprise CD3+. CD4+ or CD8+ T cells.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (αβ T cells).

In some embodiments, the method results in expansion of αβT cells over expansion of T cells expressing a TCR comprising a TCR gamma and/or TCR delta molecule, e.g., TCR gamma-delta T cells (γδ T cells).

In some embodiments, provided herein is a method of treating a disease, e.g., cancer, in a subject comprising administering to the subject an effective amount of the multifunctional polypeptide molecule as described herein.

In some embodiments, provided herein is a composition comprising the multifunctional polypeptide molecule as described herein for use in treating a disease, e.g., cancer, in a subject.

In some embodiments, provided herein is a composition comprising the multifunctional polypeptide molecule as described herein for use in the manufacture of a medicament for treating a disease, e.g., cancer, in a subject.

In some embodiments, provided herein is a method of treating a disease, e.g., cancer, in a subject comprising administering to the subject an effective amount of comprising the multifunctional polypeptide molecule as described herein, thereby treating the cancer.

In some embodiments, provided herein is a method of treating, e.g., preventing or reducing, cytokine release syndrome (CRS) and/or neurotoxicity (NT) in a subject, e.g., CRS and/or NT associated with a treatment, e.g., a previously administered treatment, comprising administering to the subject an effective amount of the multifunctional polypeptide molecule as described herein, thereby preventing CRS and/or NT in the subject.

In some embodiments, provided herein is a method of treating, e.g., preventing or reducing, cytokine release syndrome (CRS) and/or neurotoxicity (NT) in a subject, e.g., CRS and/or NT associated with a treatment, e.g., a previously administered treatment, comprising administering to the subject an effective of the multifunctional polypeptide molecule as described herein, thereby preventing CRS and/or NT in the subject.

In some embodiments, provided herein is a method of targeting a therapy, e.g., treatment, to a T cell in a subject having a disease, e.g. cancer, comprising administering an effective amount of: (i) the multifunctional polypeptide molecule as described herein; and (ii) the therapy, e.g., a tumor targeting therapy (e.g., an antibody that binds to a cancer antigen), e.g., as described herein, thereby targeting the therapy to the T cell in the subject.

In some embodiments, the method results in: reduced cytokine release syndrome (CRS) (e.g., lesser duration of CRS or no CRS), or a reduced severity of CRS (e.g., absence of severe CRS, e.g., CRS grade 4 or 5) compared to administration of (ii) alone.

In some embodiments, the multifunctional polypeptide molecule as described herein is administered concurrently with or after the administration of the treatment associated with CRS.

In some embodiments, provided herein is a method of treating a subject having a cancer, the method comprising: acquiring a value of the status of a TCRβV subfamily for the subject, wherein said value comprises a measure of the presence of, e.g., level or activity of, a TCRβV molecule in a sample from the subject, and administering an effective amount of the multifunctional polypeptide molecule as described herein: to the subject, thereby treating the subject.

In some embodiments, provided herein is a method of treating a subject having a cancer, the method comprising administering an effective amount of the multifunctional polypeptide molecule as described herein to the subject, wherein the subject has a higher, e.g., increased, level or activity of one or more TCRβV subfamilies, e.g., as described herein, compared to a reference level or activity of one or more TCRβV subfamilies, e.g., in a healthy subject, e.g., a subject not having a cancer.

In some embodiments, provided herein is a method of expanding a population of immune effector cells from a subject having a cancer, the method comprising: (i) isolating a biological sample comprising a population of immune effector cells from the subject: e.g., a peripheral blood sample, biopsy sample, or bone marrow sample; (ii) acquiring a value of the status of one or more TCRβV subfamilies for the subject, e.g., in the biological sample from the subject, wherein said value comprises a measure of the presence of, e.g., level or activity of, a TCRβV subfamily in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, the method further comprises measuring T cell function (e.g., cytotoxic activity, cytokine secretion, or degranulation) in the population of immune effector cells, e.g., compared to a reference population, e.g., an otherwise similar population not contacted with the multifunctional poly peptide molecule as described herein or a population of immune effector cells obtained from a healthy subject (e.g., a subject that does not have a cancer).

In some embodiments, the biological sample comprising the population of immune effector cells is contacted with the multifunctional polypeptide molecule as described herein identified as being higher, e.g., increased, in the biological sample.

In some embodiments, the biological sample comprising the population of immune effector cells is contacted with the multifunctional polypeptide molecule as described herein identified as being higher, e.g., increased, in the biological sample.

In some embodiments, the cancer is a solid tumor including but not limited to: melanoma, pancreatic (e.g., pancreatic adenocarcinoma) cancer, breast cancer, colorectal cancer (CRC), lung cancer (e.g., small or non-small cell lung cancer), skin cancer, ovarian cancer, or liver cancer.

In some embodiments, the cancer is a hematological cancer including, but not limited to: a B-cell or T cell malignancy, e.g., Hodgkin's lymphoma. Non-Hodgkin's lymphoma (e.g., B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome, multiple myeloma, and acute lymphocytic leukemia.

In some embodiments, the cancer is B-CLL and the TCRβV molecule comprises: (i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01; (ii) TCRβ V5 subfamily comprising TCRβ V5-6*01, TCRβ V5-4*01, or TCRβ V5-8*01; (iii) TCRβ V3 subfamily comprising TCRβ V3-1*01; (iv) TCRβ V2 subfamily comprising TCRβ V2*01; or (v) TCRβ V19 subfamily comprising TCRβ V19*01, or TCRβ V19*02.

In some embodiments, the cancer is melanoma and the TCRβV molecule comprises the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01.

In some embodiments, the cancer is DLBCL and the TCRβV molecule comprises: (i) TCRβ V13 subfamily comprising TCRβ V13*01; (ii) TCRβ V3 subfamily comprising TCRβ V3-1*01; or (iii) TCRβ V23 subfamily.

In some embodiments, the cancer is CRC and the TCRβV molecule comprises: (i) TCRβ V19 subfamily comprising TCRβ V19*01, or TCRβ V19*02 (ii) TCRβ V12 subfamily comprising TCRβ V12-4*01, TCRβ V12-3*01, or TCRβ V12-5*01 (iii) TCRβ V16 subfamily comprising TCRβ V16*01; or (iv) TCRβ V21 subfamily.

In some embodiments, the tumor comprises an antigen, e.g., a tumor antigen, e.g., a tumor associated antigen or a neoantigen; and/or the one or more TCRβV subfamilies recognize, e.g., bind to, the tumor antigen.

In some embodiments, the sample comprises a blood sample, e.g., a peripheral blood sample, a biopsy, e.g., a tumor biopsy, or a bone marrow sample.

In some embodiments, the sample comprises a biological sample comprising immune cells, e.g., TCRβV expressing cells (e.g., TCRβV+ cells), T cells, or NK cells.'

In some embodiments, the T cells comprise a CD4 T cell, a CD8 T cell, (e.g., an effector T cell or a memory T cell (e.g., a memory effector T cell (e.g., TEM cell, e.g., TEMRA cell), or a tumor infiltrating lymphocyte (TIL).

In some embodiments, the method results in an expansion, e.g., in vivo or ex vivo expansion, of at least 1.1-1000 fold, e.g., 1.1-10, 10-100, 100-200, 200-300, 300-400, 400-500, 500-600, 600-700, 700-800, 800-900, or 900-1000 fold expansion of an immune effector cell population comprising a TCRVB expressing immune effector cell, e.g., T cell.

In some embodiments, the population of cells is expanded in an appropriate media (e.g., media described herein) that includes one or more cytokines, e.g., IL-2, IL-7, IL-15, or a combination thereof.

In some embodiments, the population of cells is expanded for a period of at least about 4 hours. 6 hours, 10 hours, 12 hours. 15 hours, 18 hours, 20 hours, or 22 hours, or for at least 1, 2, 3, 4, 5, 6, 7, 8, 9, 10, 11, 12, 13, 14, 15, 16, 17, 18, 19, 20 or 21 days, or for at least about 1 week, 2 weeks, 3 weeks, 4 weeks, 5 weeks, 6 weeks, 7 weeks or 8 weeks.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule.

In some embodiments, expansion of the population of T cells having a memory-like phenotype. e.g., memory effector T cells, e.g. TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T cells having a memory-like phenotype, e.g., effector memory cells, comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+. CCR7-CD95+ T cells, optionally wherein the T cells comprise CD3+, CD4+ or CD8+ T cells.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (αβ T cells).

In some embodiments, the method results in expansion of αβT cells over expansion of T cells expressing a TCR comprising a TCR gamma and/or TCR delta molecule, e.g., TCR gamma-delta T cells (γδ T cells).

In some embodiments, the TCRβV binding moiety or molecule comprises an antigen binding domain comprising a light chain variable region (VL) comprising one, two or all of a LC CDR1, a LC CDR2 and a LC CDR3 of a VL disclosed in Tables 1, 2, 10, 11, 12 or 13.

In some embodiments, the TCRβV binding moiety or molecule comprises an antigen binding domain comprising a heavy chain variable region (VH) comprising one, two or all of a HC CDR1, a HC CDR2 and a HC CDR3 of a VH disclosed in Tables 1, 2, 10, 11, 12 or 13.

In some embodiments, the TCRβV binding moiety or molecule comprises a light chain comprising a framework region, e.g., framework region 1 (FR1), comprising one, two or all (e.g., three) of: (i) an Aspartic Acid at position 1, e.g., a substitution at position 1 according to Kabat numbering, e.g., a Alanine to Aspartic Acid substitution; or (ii) an Asparagine at position 2, e.g., a substitution at position 2 according to Kabat numbering, e.g., a Isoleucine to Asparagine, a Serine to Asparagine, or a Tyrosine to Asparagine substitution; or (iii) a Leucine at position 4, e.g., a substitution at position 4 according to Kabat numbering, e.g., a Methionine to Leucine substitution, wherein the substitution is relative to a human germline light chain framework region sequence.

In some embodiments, the TCRβV binding moiety or molecule comprises a light chain comprising a framework region, e.g., framework region 3 (FR3), comprising one, two or all (e.g., three) of: (i) a Glycine at position 66, e.g., a substitution at position 66 according to Kabat numbering, e.g., a Lysine to Glycine, or a Serine to Glycine substitution; or (ii) an Asparagine at position 69, e.g., a substitution at position 69 according to Kabat numbering, e.g., a Threonine to Asparagine substitution; or (iii) a Tyrosine at position 71, e.g., a substitution at position 71 according to Kabat numbering, e.g., a Phenylalanine to Tyrosine, or Alanine to Tyrosine substitution, wherein the substitution is relative to a human germline light chain framework region sequence.

In some embodiments, the TCRβV binding moiety or molecule binds to an outward facing region (e.g., epitope) on a TCRβV protein, e.g., as depicted by the circled area in FIG. 24A. In some embodiments, the outward facing region on the TCRβV protein comprises a structurally conserved region of TCRβV, e.g., a region of TCRβV having a similar structure across one or more TCRβV subfamilies.

In some embodiments, the method further comprises administering (e.g., sequentially, simultaneously or concurrently) a second agent, e.g., therapeutic agent, e.g., as described herein.

In some embodiments, the second agent, e.g., therapeutic agent, comprises a chemotherapeutic agent, a biologic agent, hormonal therapy), radiation, or surgery.

In some embodiments, the disease is a cancer, e.g., a solid tumor or a hematological cancer, or a metastatic lesion.

In some embodiments, the cancer antigen is BCMA or FcRH5.

In some aspects, described herein is a multifunctional polypeptide molecule comprising a first polypeptide chain, a second polypeptide chain, and at least one cytokine polypeptide or a functional fragment or a functional variant thereof, wherein the first polypeptide chain and the second polypeptide chain are non-contiguous, wherein (i) the first polypeptide chain comprises a first portion of a dimerization module linked to (A) a first TCRβV-binding moiety comprising a first heavy chain variable domain (VH) and a first light chain variable domain (VL), or a single domain antibody, or (B) a first portion of a first TCRβV-binding moiety comprising a VH of the first TCRβV-binding moiety, wherein when the first polypeptide chain comprises the first portion of the first TCRβV-binding moiety, the multifunctional polypeptide molecule further comprises a third polypeptide chain comprising a second portion of the first TCRβV-binding moiety comprising a VL of the first TCRβV-binding moiety, wherein the third polypeptide chain is non-contiguous with the first polypeptide chain and the second polypeptide chain; and (ii) the second polypeptide chain comprises a second portion of the dimerization module: wherein (a) the multifunctional poly peptide molecule comprises a single TCRβV-binding moiety and the at least one cytokine polypeptide or a functional fragment or a functional variant thereof is covalently linked to the second polypeptide chain, or (b) the multifunctional polypeptide molecule further comprises a second TCRβV-binding moiety and the at least one cytokine polypeptide or a functional fragment or a functional variant thereof is covalently linked to the first polypeptide chain, the second polypeptide chain, the third polypeptide chain when the multifunctional polypeptide molecule further comprises the third polypeptide chain, or a combination thereof.

In some embodiments, the multifunctional polypeptide molecule comprises the second TCRβV-binding moiety, and the second portion of the dimerization module is linked to: (A) a second TCRβV-binding moiety comprising a second VH and a second VL, or a single domain antibody, or (B) a first portion of a second TCRβV-binding moiety comprising a VH of the second TCRβV-binding moiety, wherein when the second polypeptide chain comprises the first portion of the second TCRβV-binding moiety, the multifunctional polypeptide molecule further comprises a fourth polypeptide chain comprising a second portion of the second TCRβV-binding moiety comprising a VL of the second TCRβV-binding moiety, wherein the fourth polypeptide chain is non-contiguous with the first polypeptide chain, the second polypeptide chain, and the third polypeptide chain: wherein the at least one cytokine polypeptide or a functional fragment or a functional variant thereof is covalently linked to the first polypeptide chain, the second polypeptide chain, the third polypeptide chain, the fourth polypeptide chain when the multifunctional polypeptide molecule further comprises the fourth polypeptide chain, or a combination thereof.

In another aspect, described herein is multifunctional polypeptide molecule comprising a first polypeptide chain, a second polypeptide chain, and at least one cytokine polypeptide or a functional fragment or a functional variant thereof, wherein the first polypeptide chain and the second polypeptide chain are non-contiguous, wherein (i) the first polypeptide chain comprises a first portion of a dimerization module linked to a first portion of a first TCRβV-binding moiety comprising a VH of the first TCRβV-binding moiety, wherein the multifunctional polypeptide molecule further comprises a third polypeptide chain comprising a second portion of the first TCRβV-binding moiety comprising a VL of the first TCRβV-binding moiety, wherein the third polypeptide chain is non-contiguous with the first polypeptide chain and the second polypeptide chain; and (ii) the second polypeptide chain comprises a second portion of the dimerization module, wherein the at least one cytokine polypeptide or a functional fragment or a functional variant thereof is covalently linked to the second polypeptide chain.

In some embodiments, the first portion of the dimerization module and the second portion of the dimerization module are dimerized.

In some embodiments, the first polypeptide chain comprises: (A) the first TCRβV-binding moiety comprising the first VH and the first VL, wherein the first TCRβV-binding moiety further comprises a first heavy chain constant domain 1 (CH1) linked to the first VH; or (B) the first portion of the first TCRβV-binding moiety comprising the VH of the first TCRβV-binding moiety, wherein the first portion of the first TCRβV-binding moiety further comprises a first CH1 linked to the VH of the first TCRβV-binding moiety.

In some embodiments, the first CH1 is linked to the C-terminus of the first VH or the C-terminus of the VH of the first TCRβV-binding moiety.

In some embodiments, the second poly peptide chain comprises: (A) the second TCRβV-binding moiety comprising the second VH and the second VL, wherein the second TCRβV-binding moiety further comprises a second CH1 linked to the second VH; or (B) the first portion of the second TCRβV-binding moiety comprising the VH of the second TCRβV-binding moiety, wherein the first portion of the second TCRβV-binding moiety further comprises a second CH1 linked to the VH of the second TCRβV-binding moiety.

In some embodiments, the second CH1 is linked to the C-terminus of the second VH or the C-terminus of the VH of the second TCRβV-binding moiety.

In some embodiments, the multifunctional polypeptide molecule comprises: (1) the first polypeptide chain comprising the first TCRβV-binding moiety that comprises the first VH and the first VL, wherein the first TCRβV-binding moiety further comprises a first light chain constant domain (CL) linked to the first VL; or (2) the first polypeptide chain comprising the first portion of the first TCRβV-binding moiety and the third polypeptide chain comprising the second portion of the first TCRβV-binding moiety, wherein the second portion of the first TCRβV-binding moiety further comprises a first CL linked to the VL of the first TCRβV-binding moiety.

In some embodiments, the first CL is linked to the C-terminus of the first VL or the C-terminus of the VL of the first TCRβV-binding moiety.

In some embodiments, the multifunctional polypeptide molecule comprises: (1) the second polypeptide chain comprising the second TCRβV-binding moiety that comprises the second VH and the second VL, wherein the second TCRβV-binding moiety further comprises a second CL linked to the second VL; or (2) the second polypeptide chain comprising the first portion of the second TCRβV-binding moiety and the fourth polypeptide chain comprising the second portion of the second TCRβV-binding moiety, wherein the second portion of the second TCRβV-binding moiety further comprises a second CL linked to the VL of the second TCRβV-binding moiety.

In some embodiments, the second CL is linked to the C-terminus of the second VL or the C-terminus of the VL of the second TCRβV-binding moiety.

In some embodiments, the first portion of the dimerization module is linked to the C-terminus of (A) the first TCRβV-binding moiety comprising the first VH and the first VL or the single domain antibody, or the C-terminus of (B) the first portion of the first TCRβV-binding moiety comprising the VH of the first TCRβV-binding moiety.

In some embodiments, the multifunctional polypeptide molecule comprises the second TCRβV-binding moiety, and the second portion of the dimerization module is linked to the C-terminus of (A) the second TCRβV-binding moiety comprising the second VH and the second VL or the single domain antibody, or the C-terminus of (B) the first portion of the second TCRβV-binding moiety comprising the VH of the second TCRβV-binding moiety.

In some embodiments, the multifunctional polypeptide molecule comprises a single TCRβV-binding moiety, and the at least one cytokine polypeptide or a functional fragment or a functional variant thereof is covalently linked to the N-terminus of the second polypeptide chain, the C-terminus of the second polypeptide chain, or a combination thereof.

In some embodiments, the at least one cytokine polypeptide or a functional fragment or a functional variant thereof is within a single contiguous polypeptide chain of the second polypeptide chain.

In some embodiments, (a) the N-terminus of the first polypeptide chain is linked to a cytokine polypeptide or a functional fragment or a functional variant thereof; the C-terminus of the first polypeptide chain is linked to a cytokine polypeptide or a functional fragment or a functional variant thereof; or a combination thereof; (b) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; (c) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; (d) the N-terminus of the fourth polypeptide chain is linked to a cytokine polypeptide or a functional fragment or a functional variant thereof; the C-terminus of the fourth polypeptide chain is linked to an cytokine polypeptide or a functional fragment or a functional variant thereof; or a combination thereof; or (e) a combination thereof.

In some embodiments. (a-1) the N-terminus of the first polypeptide chain is linked to a cytokine polypeptide or a functional fragment or a functional variant thereof; the C-terminus of the first polypeptide chain is linked to a cytokine polypeptide or a functional fragment or a functional variant thereof; or a combination thereof; and (a-2) the N-terminus of the second polypeptide chain is linked to a cytokine polypeptide or a functional fragment or a functional variant thereof; the C-terminus of the second poly peptide chain is linked to a cytokine polypeptide or a functional fragment or a functional variant thereof; or a combination thereof; (b-1) the N-terminus of the first polypeptide chain is linked to a cytokine polypeptide or a functional fragment or a functional variant thereof, the C-terminus of the first polypeptide chain is linked to a cytokine poly peptide or a functional fragment or a functional variant thereof; or a combination thereof; and (b-2) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; (c-1) the N-terminus of the first polypeptide chain is linked a cytokine polypeptide or a functional fragment or a functional variant thereof; the C-terminus of the first polypeptide chain is linked to a cytokine polypeptide or a functional fragment or a functional variant thereof; or a combination thereof; and (c-2) the N-terminus of the fourth polypeptide chain is linked to a cytokine polypeptide or a functional fragment or a functional variant thereof; the C-terminus of the fourth polypeptide chain is linked to a cytokine polypeptide or a functional fragment or a functional variant thereof; or a combination thereof; (d-1) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; and (d-2) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; (e-1) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; and (e-2) the N-terminus of the fourth polypeptide chain is linked to a cytokine polypeptide or a functional fragment or a functional variant thereof; the C-terminus of the fourth polypeptide chain is linked to a cytokine polypeptide or a functional fragment or a functional variant thereof; or a combination thereof; or (f-1) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; and (f-2) the N-terminus of the fourth polypeptide chain is linked to a cytokine polypeptide or a functional fragment or a functional variant thereof; the C-terminus of the fourth polypeptide chain is linked to a cytokine polypeptide or a functional fragment or a functional variant thereof.

In some embodiments. (a-1) the N-terminus of the first polypeptide chain is linked to a cytokine polypeptide or a functional fragment or a functional variant thereof; the C-terminus of the first polypeptide chain is linked to a cytokine polypeptide or a functional fragment or a functional variant thereof; or a combination thereof; (a-2) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; and (a-3) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; (b-1) the N-terminus of the first polypeptide chain is linked a cytokine polypeptide or a functional fragment or a functional variant thereof; the C-terminus of the first polypeptide chain is linked to a cytokine polypeptide or a functional fragment or a functional variant thereof; or a combination thereof; (b-2) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; and (b-3) the N-terminus of the fourth polypeptide chain is linked to a cytokine polypeptide or a functional fragment or a functional variant thereof; the C-terminus of the fourth polypeptide chain is linked to a cytokine polypeptide or a functional fragment or a functional variant thereof, or a combination thereof; or (c-1) the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; (c-2) the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; and (c-3) the N-terminus of the fourth polypeptide chain is linked to a cytokine poly peptide or a functional fragment or a functional variant thereof; the C-terminus of the fourth polypeptide chain is linked to a cytokine polypeptide or a functional fragment or a functional variant thereof; or a combination thereof.

In some embodiments, the N-terminus of the first polypeptide chain is linked to a cytokine polypeptide or a functional fragment or a functional variant thereof; the C-terminus of the first polypeptide chain is linked to a cytokine polypeptide or a functional fragment or a functional variant thereof; or a combination thereof; the N-terminus of the second polypeptide chain is linked to a cytokine polypeptide or a functional fragment or a functional variant thereof; the C-terminus of the second polypeptide chain is linked to a cytokine polypeptide or a functional fragment or a functional variant thereof; or a combination thereof; the N-terminus of the third polypeptide chain is linked to a cytokine polypeptide or a functional fragment or a functional variant thereof; the C-terminus of the third polypeptide chain is linked to a cytokine polypeptide or a functional fragment or a functional variant thereof; or a combination thereof; and the N-terminus of the fourth polypeptide chain is linked to a cytokine polypeptide or a functional fragment or a functional variant thereof; the C-terminus of the fourth polypeptide chain is linked to a cytokine polypeptide or a functional fragment or a functional variant thereof; or a combination thereof.

In some embodiments, the cytokine polypeptide or a functional fragment or a functional variant thereof is within a single contiguous polypeptide chain of the first polypeptide chain, the second polypeptide chain, the third cytokine poly peptide, or the fourth cytokine polypeptide to which the cytokine polypeptide or a functional fragment or a functional variant thereof is linked.

In some embodiments, the multifunctional polypeptide molecule further comprises: (i) a linker between the first portion of the dimerization module and the first TCRβV-binding moiety comprising the first VH and the first VL or the single domain antibody, or the first portion of the first TCRβV-binding moiety comprising the VH of the first TCRβV-binding moiety; (ii) a linker between the second portion of the dimerization module and the second TCRβV-binding moiety comprising the second VH and the second VL or the single domain antibody, or the first portion of the second TCRβV-binding moiety comprising the VH of the second TCRβV-binding moiety; (iii) a linker between the first VH and the first VL; (iv) a linker between the second VH and the second VL; (v) a linker between the first CH1 and the first VH, or the VH of the first TCRβV-binding moiety; (vi) a linker between the second CH1 and the second VH, or the VH of the second TCRβV-binding moiety; (vii) a linker between the first CL and the first VL, or the VL of the first TCRβV-binding moiety; (vii) a linker between the second CL and the second VL, or the VL of the second TCRβV-binding moiety; (viii) a linker between the at least one cytokine polypeptide or a functional fragment or a functional variant thereof and the first polypeptide chain, a linker between the at least one cytokine polypeptide or a functional fragment or a functional variant thereof and the second polypeptide chain, a linker between the at least one cytokine polypeptide or a functional fragment or a functional variant thereof and the third polypeptide chain, a linker between the at least one cytokine polypeptide or a functional fragment or a functional variant thereof and the fourth polypeptide chain, or a combination thereof; or (ix) a combination thereof.

In some embodiments, the linker is selected from the group consisting of a cleavable linker, a non-cleavable linker, a peptide linker, a flexible linker, a rigid linker, a helical linker, and a non-helical linker.

In some embodiments, the linker is the peptide linker and the linker comprises the sequence of SEQ ID NO: 3308 or SEQ ID NO: 3643.

In some embodiments, the multifunctional polypeptide molecule is an isolated multifunctional polypeptide molecule.

In some embodiments, the multifunctional polypeptide molecule comprises: (i) the first polypeptide chain comprising the first portion of the dimerization module linked to the C-terminus of the first portion of the first TCRβV-binding moiety; (ii) the second polypeptide chain comprising the second portion of the dimerization module. (iii) the third polypeptide chain comprising the second portion of the first TCRβV-binding moiety; and (iv) a cytokine polypeptide or a functional fragment or a functional variant thereof covalently linked to the N-terminus of the second polypeptide chain, wherein the multifunctional polypeptide molecule comprises a single TCRβV-binding moiety.

In some embodiments, the multifunctional polypeptide molecule comprises: (i) the first poly peptide chain comprising the first portion of the dimerization module linked to the C-terminus of the first portion of the first TCRβV-binding moiety; (ii) the second polypeptide chain comprising the second portion of the dimerization module linked to the C-terminus of the first portion of the second TCRβV-binding moiety; (iii) the third poly peptide chain comprising the second portion of the first TCRβV-binding moiety; (iv) the fourth polypeptide chain comprising the second portion of the second TCRβV-binding moiety; (v) a cytokine polypeptide or a functional fragment or a functional variant thereof covalently linked to the C-terminus of the third polypeptide chain, and (vi) a cytokine polypeptide or a functional fragment or a functional variant thereof covalently linked to the C-terminus of the fourth polypeptide chain.

In some embodiments, the multifunctional polypeptide molecule comprises: (i) the first polypeptide chain comprising the first portion of the dimerization module linked to the C-terminus of the first portion of the first TCRβV-binding moiety; (ii) the second polypeptide chain comprising the second portion of the dimerization module linked to the C-terminus of the first portion of the second TCRβV-binding moiety. (iii) the third poly peptide chain comprising the second portion of the first TCRβV-binding moiety; (iv) the fourth polypeptide chain comprising the second portion of the second TCRβV-binding moiety; and (v) a cytokine polypeptide or a functional fragment or a functional variant thereof covalently linked to the C-terminus of the third polypeptide chain or the C-terminus of the fourth polypeptide chain, but not to both.

In some embodiments, the multifunctional polypeptide molecule comprises: (i) the first poly peptide chain comprising the first portion of the dimerization module linked to the C-terminus of the first portion of the first TCRβV-binding moiety; (ii) the second polypeptide chain comprising the second portion of the dimerization module linked to the C-terminus of the first portion of the second TCRβV-binding moiety. (iii) the third polypeptide chain comprising the second portion of the first TCRβV-binding moiety; (iv) the fourth polypeptide chain comprising the second portion of the second TCRβV-binding moiety; and (v) a cytokine polypeptide or a functional fragment or a functional variant thereof covalently linked to the C-terminus of the first polypeptide chain or the C-terminus of the second polypeptide chain, but not to both.

In some embodiments, the first TCRβV-binding moiety, the second TCRβV-binding moiety, or a combination thereof comprises any one selected from the group consisting of a Fab, a F(ab′)2, an Fv, a single chain Fv (scFv), a single domain antibody, a diabody (dAb), a camelid antibody, and a combination thereof.

In some embodiments, the first TCRβV-binding moiety, the second TCRβV-binding moiety, or a combination thereof comprises the Fab or the scFv.

In some embodiments, the TCRβV-binding moiety is the sole antigen-binding moiety of the multifunctional polypeptide molecule.

In some embodiments, the multifunctional polypeptide molecule comprises two or more of the at least one cytokine polypeptides.

In some embodiments, the at least one cytokine polypeptide comprises interleukin-2 (IL-2) or a fragment thereof.

In some embodiments, the at least one cytokine polypeptide comprises a sequence having at least 75% sequence identity to the sequence of SEQ ID NO: 2191.

In some embodiments, the variant is an IL-2 variant comprising a substitution mutation.

In some embodiments, the variant is an IL-2 variant comprising C125A mutation.

In some embodiments, the variant comprises a sequence having at least 75% sequence identity to the sequence of SEQ ID NO: 2270.

In some embodiments, the first portion of the dimerization module comprises a first immunoglobulin constant regions (Fc regions) and the second portion of the dimerization module comprises a second Fc region.

In some embodiments, the first Fc region, the second Fc region, or a combination thereof is selected from the group consisting of an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from the group consisting of a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRβV-binding moiety, the second TCRβV-binding moiety, or a combination thereof binds to one or more of a TCRβV subfamily selected from the group consisting of: (i) TCRβ V2 subfamily comprising TCRβ V2*01; (ii) TCRβ V3 subfamily comprising TCRβ V3-1*01; (iii) TCRβ V4 subfamily comprising one or more selected from TCRβ V4-1, TCRβ V4-2, and TCRβ V4-3; (iv) TCRβ V5 subfamily comprising one or more selected from TCRβ V5-6*01, TCRβ V5-4*01, TCRβ V5-1*01, and TCRβ V5-8*01; (v) the TCRβ V6 subfamily comprising one or more selected from TCRβ V6-4*01. TCRβ V6-4*02, TCRβ V6-9*01, TCRβ V6-8*01, TCRβ V6-5*01, TCRβ V6-6*02, TCRβ V6-6*01, TCRB V6-2*01, TCRβ V6-3*01, and TCRβ V6-1*01; (vi) TCRβ V9 subfamily; (vii) TCRβ V10 subfamily comprising one or more selected from TCRβ V10-1*01, TCRβ V10-1*02, TCRβ V10-3*01, and TCRβ V10-2*01; (viii) TCRβ V11 subfamily comprising TCRβ V11-2; (ix) TCRβ V12 subfamily comprising one or more selected from TCRβ V12-4*01, TCRβ V12-3*01, and TCRβ V12-5*01: (x) TCRβ V13 subfamily comprising TCRβ V13*01: (xi) TCRβ V16 subfamily comprising TCRβ V16*01: (xii) TCRβ V19 subfamily comprising one or more selected from TCRβ V19*01 and TCRβ V19*02; (xiii) TCRβ V21 subfamily: (xiv) TCRβ V23 subfamily: (xv) TCRβ V27 subfamily; and (xvi) TCRβ V28 subfamily.

In some embodiments, the multifunctional polypeptide molecule comprises the first TCRβV-binding moiety and the second TCRβV-binding moiety, and the first TCRβV-binding moiety and the second TCRβV-binding moiety are same.

In some embodiments, the multifunctional polypeptide molecule comprises the first TCRβV-binding moiety and the second TCRβV-binding moiety, and the first TCRβV-binding moiety and the second TCRβV-binding moiety are different.

In some embodiments, the first TCRβV-binding moiety and the second TCRβV-binding moiety binds: (i) one or more of a TCRβ V6 subfamily member and one or more of a TCRβ V10 subfamily member, respectively. (ii) one or more of a TCRβ V6 subfamily member and one or more of a TCRβ V5 subfamily member, respectively; (iii) one or more of a TCRβ V6 subfamily member and one or more of a TCRβ V12 subfamily member, respectively; (iv) one or more of a TCRβ V10 subfamily member and one or more of a TCRβ V5 subfamily member, respectively; (v) one or more of a TCRβ V10 subfamily member and one or more of a TCRβ V12 subfamily member, respectively; or (vi) one or more of a TCRβ V5 subfamily member and one or more of a TCRβ V12 subfamily member, respectively.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75% sequence identity to any one of the CDR1, CDR2, and CDR3 sequences listed in Table 1; (ii) a LC CDR1, a LC CDR2, and a LC CDR3 of an amino acid sequence having at least 75% sequence identity to any one of the CDR1, CDR2, and CDR3 the sequences listed in Table 1; or (iii) a combination thereof.

In some embodiments, the first TCRβV-binding moiety, the second TCRβV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75% sequence identity to a non-murine germline FR1, a non-murine germline FR2, a non-murine germline FR3, and a non-murine germline FR4; (ii) a VL comprising a FR comprising a FR1, a FR2, a FR3, and a FR4 that have at least 75% sequence identity to a non-murine germline FR1, a non-murine germline FR2, a non-murine germline FR3, and a non-murine germline FR4; or (iii) a combination thereof.

In some embodiments, the VH comprises the FR3 comprising (i) a Threonine at position 73 according to Kabat numbering; (ii) a Glycine a position 94 according to Kabat numbering; or (iii) a combination thereof.

In some embodiments, the VL comprises the FR1 comprising a Phenylalanine at position 10 according to Kabat numbering.

In some embodiments, the VL comprises the FR2 comprising (i) a Histidine at position 36 according to Kabat numbering; (ii) an Alanine at position 46 according to Kabat numbering; or (iii) a combination thereof.

In some embodiments, the VL comprises the FR3 comprising a Phenylalanine at position 87 according to Kabat numbering.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75% sequence identity to any one of the CDR1, CDR2, and CDR3 sequences listed in Table 2; (ii) a LC CDR1, a LC CDR2, and a LC CDR3 of an amino acid sequence having at least 75% sequence identity to any one of the CDR1, CDR2, and CDR3 sequences listed in Table 2; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75% sequence identity to a FR1, a FR2, a FR3, and a FR4 of a humanized B-H LC of Table 2; (ii) a VL comprising a FR comprising a FR1, a FR2, a FR3, and a FR4 that have at least 75% sequence identity to a FR1, a FR2, a FR3, and a FR4 of a humanized B-H LC of Table 2; or (iii) a combination thereof.

In some embodiments, the first TCRβV-binding moiety, the second TCRβV-binding moiety, or a combination thereof comprises: (i) a VH comprising a sequence having at least 75% sequence identity to the VH sequence of a humanized Antibody B-H listed in Table 2; (ii) a VL comprising a sequence having at least 75% sequence identity to the VL sequence of a humanized Antibody B-H listed in Table 2; or (iii) a combination thereof.

In some embodiments, the first polypeptide chain, the second polypeptide chain, or a combination thereof comprises a heavy chain constant region having a sequence having at least 75% sequence identity to any one of the sequences listed in Table 3, 21, or 22 or a combination thereof.

In some embodiments, the first polypeptide chain, the second polypeptide chain, or a combination thereof comprises a heavy chain constant region of an IgM or a fragment thereof.

In some embodiments, the heavy chain constant region of the IgM comprises a sequence having at least 75% sequence identity to the sequence of SEQ ID NO: 73.

In some embodiments, the first polypeptide chain, the second poly peptide chain, or a combination thereof comprises a heavy chain constant region of an IgJ or a fragment thereof.

In some embodiments, the heavy chain constant region of the IgJ comprises a sequence having at least 75% sequence identity to the sequence of SEQ ID NO: 76.

In some embodiments, the first polypeptide chain, the second polypeptide chain, a combination thereof comprises a heavy chain constant region of an IgGA1 or a fragment thereof.

In some embodiments, the heavy chain constant region of the IgGA1 comprises a sequence having at least 75% sequence identity to the sequence of SEQ ID NO. 74.

In some embodiments, the first polypeptide chain, the second poly peptide chain, or a combination thereof comprises a heavy chain constant region of an IgGA2 or a fragment thereof.

In some embodiments, the heavy chain constant region of the IgGA2 comprises a sequence having at least 75% sequence identity to the sequence of SEQ ID NO: 75.

In some embodiments, the first polypeptide chain, the second polypeptide chain, or a combination thereof comprises a heavy chain constant region of an IgG1 or a fragment thereof.

In some embodiments, the heavy chain constant region of the IgG1 comprises a sequence having at least 75% sequence identity to the sequence of SEQ ID NO: 41 or SEQ ID NO. 3645.

In some embodiments, the first polypeptide chain, the second polypeptide chain, the third polypeptide chain, the fourth polypeptide chain, or a combination thereof comprises a light chain constant region having a sequence having at least 75% sequence identity to any one of the sequences listed in Table 3, 21, or 22 or a combination thereof.

In some embodiments, the first polypeptide chain, the second polypeptide chain, the third polypeptide chain, the fourth polypeptide chain, or a combination thereof comprises a light chain constant region of a kappa chain or a fragment thereof.

In some embodiments, the light chain constant region of a kappa chain comprises a light chain constant region sequence listed in Table 3, 21, or 22.

In some embodiments, the light chain constant region of the kappa chain comprises a sequence having at least 75% sequence identity to the sequence of SEQ ID NO: 39 or SEQ ID NO: 3644.

In some embodiments, the first TCRβV-binding moiety, the second TCRβV-binding moiety, or a combination thereof comprises: (i) a HC CDR1, a HC CDR2 and a HC CDR3 comprising amino acid sequences having at least 75% sequence identity to CDR1, CDR2, and CDR3 sequences of a VH disclosed in Tables 1, 2, 10, 11, 12 or 13; (ii) a LC CDR1, a LC CDR2, and a LC CDR3 comprising an amino acid sequence having at least 75% sequence identity to CDR1, CDR2, and CDR3 sequences of a VL disclosed in Tables 1, 2, 10, 11, 12 or 13; or (iii) a combination thereof.

In some embodiments, the first TCRβV-binding moiety, the second TCRβV-binding moiety, or a combination thereof comprises a light chain comprising a FR1 comprising: (i) an Aspartic Acid at position 1 according to Kabat numbering; (ii) an Asparagine at position 2 according to Kabat numbering; (iii) a Leucine at position 4 according to Kabat numbering; or (iv) a combination thereof.

In some embodiments, the first TCRβV-binding moiety, the second TCRβV-binding moiety, or a combination thereof comprises a light chain comprising a FR3 comprising: (i) a Glycine at position 66 according to Kabat numbering; (ii) an Asparagine at position 69 according to Kabat numbering; (iii) a Tyrosine at position 71 according to Kabat numbering; or (iv) a combination thereof.

In some embodiments, the first TCRβV-binding moiety, the second TCRβV-binding moiety, or a combination thereof binds to an outward facing region on a TCRβV protein.

In some embodiments, the outward facing region on the TCRβV protein comprises a structurally conserved region of TCRβV having a similar structure across one or more TCRβV subfamilies.

In some embodiments, the first polypeptide chain, the second polypeptide chain, or a combination thereof comprises (i) a first sequence selected from the group consisting of SEQ ID NOS: 80, 83, 86, 89, 92, 95, 98, 101, 104, 107, 110, 110, 113, 116, 119, 122, 125, 128, 131, 134, 137, 140, 143, 146, 149, 153, 156, 159, 162, 165, 168, 171, 174, 177, 180, 183, 186, 189, 192, 195, 198, 201, 204, 206, 208, 210, 212, 214, 216, 218, 220, 222, 224, 1309, 1326, 1327, 1328, 1329, 1330, 1331, 1332, 1333, 1334, 1335, 1336, 1337, 1338, 1339, 1340, 1341, 1342, 3281, and 3642; and (ii) a second sequence selected from the group consisting of SEQ ID NOS: 40, 41, 42, 73, 74, 75, 76, 3645, 3646, 3647, 3648, and 3649; wherein the first sequence is linked to the second sequence.

In some embodiments, the first polypeptide chain, the second polypeptide chain, or a combination thereof further comprises a third sequence selected from the group consisting of SEQ ID NO: 2191 and SEQ ID NO: 2270, wherein the third sequence is linked to the first sequence, the second sequence, or a combination thereof.

In some embodiments, the third sequence is linked to the N-terminus of the first sequence.

In some embodiments, the third sequence is linked to the C-terminus of the second sequence.

In some embodiments, the first polypeptide chain, the second poly peptide chain, or a combination thereof comprises (i) a first sequence selected from the group consisting of SEQ ID NOS: 1, 9, 15, 23, 25, 82, 85, 88, 91, 94, 97, 100, 103, 106, 109, 112, 115, 118, 121, 124, 127, 130, 133, 136, 139, 142, 145, 148, 151, 155, 158, 161, 164, 167, 170, 173, 176, 179, 182, 185, 188, 191, 194, 197, 200, 203, 205, 207, 209, 211, 213, 215, 217, 219, 221, 223, 225, 1100, 1310, 1311, 1312, 1344, 1346, 1348, 1350, 1356, 1360, 1362, 1370, and 3438; and (ii) a second sequence selected from the group consisting of SEQ ID NOS: 40, 41, 42, 73, 74, 75, 76, 3645, 3646, 3647, 3648, and 3649; wherein the first sequence is linked to the second sequence.

In some embodiments, the first polypeptide chain, the second polypeptide chain, or a combination thereof further comprises a third sequence selected from the group consisting of SEQ ID NO: 2191 and SEQ ID NO: 2270, wherein the third sequence is linked to the first sequence, the second sequence, or a combination thereof.

In some embodiments, the third sequence is linked to the N-terminus of the first sequence.

In some embodiments, the third sequence is linked to the C-terminus of the second sequence.

In some embodiments, the third polypeptide chain, the fourth polypeptide chain, or a combination thereof comprises (i) a fourth sequence selected from the group consisting of SEQ ID NOS: 2, 10, 11, 16, 26, 27, 28, 29, 30, 81, 84, 87, 90, 93, 96, 99, 102, 105, 108, 111, 114, 117, 120, 123, 126, 129, 132, 135, 138, 141, 144, 147, 150, 154, 157, 160, 163, 166, 169, 172, 175, 178, 181, 184, 187, 190, 193, 196, 199, 202, 1101, 1313, 1314, 1347, 1349, 1351, 1353, 1357, 1361, 1365, 1367, 1369, and 3279; and (ii) a fifth sequence selected from the group consisting of SEQ ID NOS: 39 and 3644, wherein the fourth sequence is linked to the fifth sequence.

In some embodiments, the third polypeptide chain, the fourth polypeptide chain, or a combination thereof further comprises the third sequence, wherein the third sequence is linked to the fourth sequence, the fifth sequence, or a combination thereof.

In some embodiments, the third sequence is linked to the N-terminus of the fourth sequence.

In some embodiments, the third sequence is linked to the C-terminus of the fifth sequence.

In some embodiments, the first polypeptide chain, the second polypeptide chain, or a combination thereof comprises: a first sequence of SEQ ID NO: 1 linked to a second sequence of SEQ ID NO: 40; a first sequence of SEQ ID NO: 1 linked to a second sequence of SEQ ID NO: 42; a first sequence of SEQ ID NO: 1 linked to a second sequence of SEQ ID NO: 74; a first sequence of SEQ ID NO: 1 linked to a second sequence of SEQ ID NO: 3645; a first sequence of SEQ ID NO: 1 linked to a second sequence of SEQ ID NO: 3646; a first sequence of SEQ ID NO: 1 linked to a second sequence of SEQ ID NO: 3648; a first sequence of SEQ ID NO: 1 linked to a second sequence of SEQ ID NO: 3649; a first sequence of SEQ ID NO: 9 linked to a second sequence of SEQ ID NO: 40; a first sequence of SEQ ID NO: 9 linked to a second sequence of SEQ ID NO. 42; a first sequence of SEQ ID NO. 9 linked to a second sequence of SEQ ID NO: 74; a first sequence of SEQ ID NO: 9 linked to a second sequence of SEQ ID NO: 3645; a first sequence of SEQ ID NO: 9 linked to a second sequence of SEQ ID NO: 3646; a first sequence of SEQ ID NO: 9 linked to a second sequence of SEQ ID NO: 3648; a first sequence of SEQ ID NO: 9 linked to a second sequence of SEQ ID NO: 3649; a first sequence of SEQ ID NO: 25 linked to a second sequence of SEQ ID NO: 40; a first sequence of SEQ ID NO: 25 linked to a second sequence of SEQ ID NO: 42; a first sequence of SEQ ID NO: 25 linked to a second sequence of SEQ ID NO: 74; a first sequence of SEQ ID NO: 25 linked to a second sequence of SEQ ID NO: 3645; a first sequence of SEQ ID NO: 25 linked to a second sequence of SEQ ID NO: 3646; a first sequence of SEQ ID NO: 25 linked to a second sequence of SEQ ID NO: 3648; a first sequence of SEQ ID NO: 25 linked to a second sequence of SEQ ID NO: 3649; a first sequence of SEQ ID NO: 82 linked to a second sequence of SEQ ID NO: 40; a first sequence of SEQ ID NO: 82 linked to a second sequence of SEQ ID NO: 42; a first sequence of SEQ ID NO: 82 linked to a second sequence of SEQ ID NO: 74; a first sequence of SEQ ID NO: 82 linked to a second sequence of SEQ ID NO: 3645; a first sequence of SEQ ID NO: 82 linked to a second sequence of SEQ ID NO: 3646; a first sequence of SEQ ID NO: 82 linked to a second sequence of SEQ ID NO: 3648; a first sequence of SEQ ID NO: 82 linked to a second sequence of SEQ ID NO: 3649; a first sequence of SEQ ID NO: 91 linked to a second sequence of SEQ ID NO: 40; a first sequence of SEQ ID NO: 91 linked to a second sequence of SEQ ID NO: 42; a first sequence of SEQ ID NO: 91 linked to a second sequence of SEQ ID NO: 74, a first sequence of SEQ ID NO: 91 linked to a second sequence of SEQ ID NO: 3645; a first sequence of SEQ ID NO: 91 linked to a second sequence of SEQ ID NO: 3646; a first sequence of SEQ ID NO: 91 linked to a second sequence of SEQ ID NO: 3648; a first sequence of SEQ ID NO: 91 linked to a second sequence of SEQ ID NO: 3649; a first sequence of SEQ ID NO: 103 linked to a second sequence of SEQ ID NO: 40; a first sequence of SEQ ID NO: 103 linked to a second sequence of SEQ ID NO: 42; a first sequence of SEQ ID NO: 103 linked to a second sequence of SEQ ID NO: 74; a first sequence of SEQ ID NO: 103 linked to a second sequence of SEQ ID NO: 3645; a first sequence of SEQ ID NO: 103 linked to a second sequence of SEQ ID NO: 3646; a first sequence of SEQ ID NO: 103 linked to a second sequence of SEQ ID NO: 3648; a first sequence of SEQ ID NO. 103 linked to a second sequence of SEQ ID NO: 3649; a first sequence of SEQ ID NO: 118 linked to a second sequence of SEQ ID NO: 40; a first sequence of SEQ ID NO: 118 linked to a second sequence of SEQ ID NO: 42; a first sequence of SEQ ID NO: 118 linked to a second sequence of SEQ ID NO: 74; a first sequence of SEQ ID NO: 118 linked to a second sequence of SEQ ID NO: 3645; a first sequence of SEQ ID NO: 118 linked to a second sequence of SEQ ID NO: 3646; a first sequence of SEQ ID NO: 118 linked to a second sequence of SEQ ID NO: 3648; a first sequence of SEQ ID NO: 118 linked to a second sequence of SEQ ID NO: 3649; a first sequence of SEQ ID NO: 130 linked to a second sequence of SEQ ID NO: 40; a first sequence of SEQ ID NO: 130 linked to a second sequence of SEQ ID NO: 42; a first sequence of SEQ ID NO: 130 linked to a second sequence of SEQ ID NO: 74; a first sequence of SEQ ID NO: 130 linked to a second sequence of SEQ ID NO: 3645; a first sequence of SEQ ID NO: 130 linked to a second sequence of SEQ ID NO: 3646; a first sequence of SEQ ID NO. 130 linked to a second sequence of SEQ ID NO: 3648; a first sequence of SEQ ID NO: 130 linked to a second sequence of SEQ ID NO: 3649; a first sequence of SEQ ID NO: 142 linked to a second sequence of SEQ ID NO: 40; a first sequence of SEQ ID NO. 142 linked to a second sequence of SEQ ID NO: 42; a first sequence of SEQ ID NO: 142 linked to a second sequence of SEQ ID NO: 74; a first sequence of SEQ ID NO: 142 linked to a second sequence of SEQ ID NO: 3645; a first sequence of SEQ ID NO: 142 linked to a second sequence of SEQ ID NO: 3646; a first sequence of SEQ ID NO: 142 linked to a second sequence of SEQ ID NO: 3648; a first sequence of SEQ ID NO: 142 linked to a second sequence of SEQ ID NO: 3649; a first sequence of SEQ ID NO: 151 linked to a second sequence of SEQ ID NO: 40: a first sequence of SEQ ID NO: 151 linked to a second sequence of SEQ ID NO: 42; a first sequence of SEQ ID NO: 151 linked to a second sequence of SEQ ID NO: 74; a first sequence of SEQ ID NO: 151 linked to a second sequence of SEQ ID NO: 3645; a first sequence of SEQ ID NO: 151 linked to a second sequence of SEQ ID NO: 3646; a first sequence of SEQ ID NO: 151 linked to a second sequence of SEQ ID NO: 3648: a first sequence of SEQ ID NO: 151 linked to a second sequence of SEQ ID NO: 3649; a first sequence of SEQ ID NO: 167 linked to a second sequence of SEQ ID NO: 40; a first sequence of SEQ ID NO: 167 linked to a second sequence of SEQ ID NO: 42; a first sequence of SEQ ID NO: 167 linked to a second sequence of SEQ ID NO: 74; a first sequence of SEQ ID NO: 167 linked to a second sequence of SEQ ID NO: 3645; a first sequence of SEQ ID NO: 167 linked to a second sequence of SEQ ID NO: 3646; a first sequence of SEQ ID NO: 167 linked to a second sequence of SEQ ID NO: 3648; a first sequence of SEQ ID NO: 167 linked to a second sequence of SEQ ID NO: 3649; a first sequence of SEQ ID NO: 182 linked to a second sequence of SEQ ID NO: 40; a first sequence of SEQ ID NO. 182 linked to a second sequence of SEQ ID NO: 42; a first sequence of SEQ ID NO: 182 linked to a second sequence of SEQ ID NO: 74; a first sequence of SEQ ID NO: 182 linked to a second sequence of SEQ ID NO: 3645; a first sequence of SEQ ID NO: 182 linked to a second sequence of SEQ ID NO: 3646; a first sequence of SEQ ID NO: 182 linked to a second sequence of SEQ ID NO: 3648; a first sequence of SEQ ID NO: 182 linked to a second sequence of SEQ ID NO: 3649; a first sequence of SEQ ID NO: 197 linked to a second sequence of SEQ ID NO: 40; a first sequence of SEQ ID NO: 197 linked to a second sequence of SEQ ID NO: 42; a first sequence of SEQ ID NO: 197 linked to a second sequence of SEQ ID NO: 74; a first sequence of SEQ ID NO: 197 linked to a second sequence of SEQ ID NO: 3645; a first sequence of SEQ ID NO: 197 linked to a second sequence of SEQ ID NO: 3646; a first sequence of SEQ ID NO: 197 linked to a second sequence of SEQ ID NO: 3648; a first sequence of SEQ ID NO: 197 linked to a second sequence of SEQ ID NO: 3649; a first sequence of SEQ ID NO: 203 linked to a second sequence of SEQ ID NO: 40; a first sequence of SEQ ID NO: 203 linked to a second sequence of SEQ ID NO: 42; a first sequence of SEQ ID NO: 203 linked to a second sequence of SEQ ID NO: 74; a first sequence of SEQ ID NO. 203 linked to a second sequence of SEQ ID NO: 3645; a first sequence of SEQ ID NO: 203 linked to a second sequence of SEQ ID NO: 3646; a first sequence of SEQ ID NO: 203 linked to a second sequence of SEQ ID NO: 3648; a first sequence of SEQ ID NO: 203 linked to a second sequence of SEQ ID NO: 3649; a first sequence of SEQ ID NO: 209 linked to a second sequence of SEQ ID NO: 40: a first sequence of SEQ ID NO: 209 linked to a second sequence of SEQ ID NO. 42; a first sequence of SEQ ID NO: 209 linked to a second sequence of SEQ ID NO: 74; a first sequence of SEQ ID NO: 209 linked to a second sequence of SEQ ID NO: 3645; a first sequence of SEQ ID NO: 209 linked to a second sequence of SEQ ID NO: 3646; a first sequence of SEQ ID NO: 209 linked to a second sequence of SEQ ID NO: 3648; a first sequence of SEQ ID NO: 209 linked to a second sequence of SEQ ID NO: 3649; a first sequence of SEQ ID NO: 215 linked to a second sequence of SEQ ID NO: 40; a first sequence of SEQ ID NO: 215 linked to a second sequence of SEQ ID NO: 42; a first sequence of SEQ ID NO: 215 linked to a second sequence of SEQ ID NO: 74; a first sequence of SEQ ID NO: 215 linked to a second sequence of SEQ ID NO: 3645; a first sequence of SEQ ID NO: 215 linked to a second sequence of SEQ ID NO: 3646; a first sequence of SEQ ID NO: 215 linked to a second sequence of SEQ ID NO: 3648; a first sequence of SEQ ID NO: 215 linked to a second sequence of SEQ ID NO: 3649; a first sequence of SEQ ID NO: 221 linked to a second sequence of SEQ ID NO: 40; a first sequence of SEQ ID NO: 221 linked to a second sequence of SEQ ID NO: 42; a first sequence of SEQ ID NO: 221 linked to a second sequence of SEQ ID NO: 74; a first sequence of SEQ ID NO: 221 linked to a second sequence of SEQ ID NO: 3645; a first sequence of SEQ ID NO. 221 linked to a second sequence of SEQ ID NO: 3646; a first sequence of SEQ ID NO: 221 linked to a second sequence of SEQ ID NO: 3648; a first sequence of SEQ ID NO: 221 linked to a second sequence of SEQ ID NO: 3649; a first sequence of SEQ ID NO: 1100 linked to a second sequence of SEQ ID NO: 40; a first sequence of SEQ ID NO: 1100 linked to a second sequence of SEQ ID NO: 42, a first sequence of SEQ ID NO: 1100 linked to a second sequence of SEQ ID NO: 74; a first sequence of SEQ ID NO: 1100 linked to a second sequence of SEQ ID NO: 3645; a first sequence of SEQ ID NO: 1100 linked to a second sequence of SEQ ID NO: 3646; a first sequence of SEQ ID NO: 1100 linked to a second sequence of SEQ ID NO: 3648; a first sequence of SEQ ID NO: 1100 linked to a second sequence of SEQ ID NO: 3649; a first sequence of SEQ ID NO: 1310 linked to a second sequence of SEQ ID NO. 40; a first sequence of SEQ ID NO. 1310 linked to a second sequence of SEQ ID NO: 42; a first sequence of SEQ ID NO: 1310 linked to a second sequence of SEQ ID NO: 74; a first sequence of SEQ ID NO: 1310 linked to a second sequence of SEQ ID NO: 3645; a first sequence of SEQ ID NO: 1310 linked to a second sequence of SEQ ID NO: 3646; a first sequence of SEQ ID NO: 1310 linked to a second sequence of SEQ ID NO: 3648; a first sequence of SEQ ID NO: 1310 linked to a second sequence of SEQ ID NO: 3649; a first sequence of SEQ ID NO: 1346 linked to a second sequence of SEQ ID NO: 40; a first sequence of SEQ ID NO: 1346 linked to a second sequence of SEQ ID NO: 42; a first sequence of SEQ ID NO: 1346 linked to a second sequence of SEQ ID NO: 74; a first sequence of SEQ ID NO: 1346 linked to a second sequence of SEQ ID NO: 3645; a first sequence of SEQ ID NO. 1346 linked to a second sequence of SEQ ID NO: 3646; a first sequence of SEQ ID NO: 1346 linked to a second sequence of SEQ ID NO: 3648; a first sequence of SEQ ID NO: 1346 linked to a second sequence of SEQ ID NO: 3649; a first sequence of SEQ ID NO: 1350 linked to a second sequence of SEQ ID NO: 40; a first sequence of SEQ ID NO: 1350 linked to a second sequence of SEQ ID NO: 42, a first sequence of SEQ ID NO: 1350 linked to a second sequence of SEQ ID NO: 74; a first sequence of SEQ ID NO: 1350 linked to a second sequence of SEQ ID NO: 3645; a first sequence of SEQ ID NO: 1350 linked to a second sequence of SEQ ID NO: 3646; a first sequence of SEQ ID NO: 1350 linked to a second sequence of SEQ ID NO: 3648; a first sequence of SEQ ID NO: 1350 linked to a second sequence of SEQ ID NO: 3649; a first sequence of SEQ ID NO: 1360 linked to a second sequence of SEQ ID NO: 40; a first sequence of SEQ ID NO: 1360 linked to a second sequence of SEQ ID NO: 42; a first sequence of SEQ ID NO: 1360 linked to a second sequence of SEQ ID NO: 74; a first sequence of SEQ ID NO: 1360 linked to a second sequence of SEQ ID NO: 3645; a first sequence of SEQ ID NO: 1360 linked to a second sequence of SEQ ID NO: 3646; a first sequence of SEQ ID NO: 1360 linked to a second sequence of SEQ ID NO: 3648; a first sequence of SEQ ID NO: 1360 linked to a second sequence of SEQ ID NO: 3649; a first sequence of SEQ ID NO: 1370 linked to a second sequence of SEQ ID NO: 40; a first sequence of SEQ ID NO: 1370 linked to a second sequence of SEQ ID NO: 42; a first sequence of SEQ ID NO: 1370 linked to a second sequence of SEQ ID NO: 74; a first sequence of SEQ ID NO: 1370 linked to a second sequence of SEQ ID NO: 3645; a first sequence of SEQ ID NO. 1370 linked to a second sequence of SEQ ID NO: 3646; a first sequence of SEQ ID NO: 1370 linked to a second sequence of SEQ ID NO: 3648; or a first sequence of SEQ ID NO: 1370 linked to a second sequence of SEQ ID NO: 3649.

In some embodiments, the first polypeptide chain, the second polypeptide chain, or a combination thereof further comprises a third sequence selected from the group consisting of SEQ ID NO: 2191 and SEQ ID NO: 2270, wherein the third sequence is linked to the first sequence, the second sequence, or a combination thereof.

In some embodiments, the third sequence is linked to the N-terminus of the first sequence.

In some embodiments, the third sequence is linked to the C-terminus of the second sequence.

In some embodiments, the third polypeptide chain, the fourth polypeptide chain, or a combination thereof comprises: a fourth sequence of SEQ ID NO: 2 linked to a fifth sequence of SEQ ID NO: 3644: a fourth sequence of SEQ ID NO: 2 linked to a fifth sequence of SEQ ID NO: 39: a fourth sequence of SEQ ID NO: 10 linked to a fifth sequence of SEQ ID NO: 3644; a fourth sequence of SEQ ID NO: 10 linked to a fifth sequence of SEQ ID NO: 39: a fourth sequence of SEQ ID NO: 16 linked to a fifth sequence of SEQ ID NO: 3644; a fourth sequence of SEQ ID NO: 16 linked to a fifth sequence of SEQ ID NO: 39: a fourth sequence of SEQ ID NO: 28 linked to a fifth sequence of SEQ ID NO: 3644: a fourth sequence of SEQ ID NO: 28 linked to a fifth sequence of SEQ ID NO: 39, a fourth sequence of SEQ ID NO: 87 linked to a fifth sequence of SEQ ID NO: 3644; a fourth sequence of SEQ ID NO: 87 linked to a fifth sequence of SEQ ID NO: 39: a fourth sequence of SEQ ID NO: 90 linked to a fifth sequence of SEQ ID NO: 3644; a fourth sequence of SEQ ID NO: 90 linked to a fifth sequence of SEQ ID NO: 39: a fourth sequence of SEQ ID NO: 96 linked to a fifth sequence of SEQ ID NO: 3644, a fourth sequence of SEQ ID NO. 96 linked to a fifth sequence of SEQ ID NO: 39: a fourth sequence of SEQ ID NO: 105 linked to a fifth sequence of SEQ ID NO: 3644: a fourth sequence of SEQ ID NO: 105 linked to a fifth sequence of SEQ ID NO: 39: a fourth sequence of SEQ ID NO 117 linked to a fifth sequence of SEQ ID NO: 3644; a fourth sequence of SEQ ID NO: 117 linked to a fifth sequence of SEQ ID NO: 39: a fourth sequence of SEQ ID NO: 120 linked to a fifth sequence of SEQ ID NO: 3644: a fourth sequence of SEQ ID NO: 120 linked to a fifth sequence of SEQ ID NO: 39; a fourth sequence of SEQ ID NO: 129 linked to a fifth sequence of SEQ ID NO: 3644: a fourth sequence of SEQ ID NO: 129 linked to a fifth sequence of SEQ ID NO: 39; a fourth sequence of SEQ ID NO: 132 linked to a fifth sequence of SEQ ID NO: 3644: a fourth sequence of SEQ ID NO: 132 linked to a fifth sequence of SEQ ID NO: 39: a fourth sequence of SEQ ID NO: 141 linked to a fifth sequence of SEQ ID NO: 3644: a fourth sequence of SEQ ID NO: 141 linked to a fifth sequence of SEQ ID NO: 39: a fourth sequence of SEQ ID NO: 150 linked to a fifth sequence of SEQ ID NO: 3644: a fourth sequence of SEQ ID NO: 150 linked to a fifth sequence of SEQ ID NO: 39: a fourth sequence of SEQ ID NO: 154 linked to a fifth sequence of SEQ ID NO: 3644; a fourth sequence of SEQ ID NO: 154 linked to a fifth sequence of SEQ ID NO: 39: a fourth sequence of SEQ ID NO: 163 linked to a fifth sequence of SEQ ID NO: 3644; a fourth sequence of SEQ ID NO: 163 linked to a fifth sequence of SEQ ID NO: 39: a fourth sequence of SEQ ID NO: 169 linked to a fifth sequence of SEQ ID NO: 3644: a fourth sequence of SEQ ID NO: 169 linked to a fifth sequence of SEQ ID NO: 39; a fourth sequence of SEQ ID NO: 175 linked to a fifth sequence of SEQ ID NO: 3644: a fourth sequence of SEQ ID NO: 175 linked to a fifth sequence of SEQ ID NO: 39; a fourth sequence of SEQ ID NO: 181 linked to a fifth sequence of SEQ ID NO: 3644: a fourth sequence of SEQ ID NO. 181 linked to a fifth sequence of SEQ ID NO: 39; a fourth sequence of SEQ ID NO: 187 linked to a fifth sequence of SEQ ID NO: 3644; a fourth sequence of SEQ ID NO. 187 linked to a fifth sequence of SEQ ID NO: 39; a fourth sequence of SEQ ID NO: 193 linked to a fifth sequence of SEQ ID NO. 3644: a fourth sequence of SEQ ID NO: 193 linked to a fifth sequence of SEQ ID NO: 39; a fourth sequence of SEQ ID NO: 202 linked to a fifth sequence of SEQ ID NO: 3644; a fourth sequence of SEQ ID NO: 202 linked to a fifth sequence of SEQ ID NO: 39; a fourth sequence of SEQ ID NO: 1101 linked to a fifth sequence of SEQ ID NO: 3644: a fourth sequence of SEQ ID NO: 1101 linked to a fifth sequence of SEQ ID NO: 39: a fourth sequence of SEQ ID NO: 1349 linked to a fifth sequence of SEQ ID NO: 3644: a fourth sequence of SEQ ID NO: 1349 linked to a fifth sequence of SEQ ID NO: 39; a fourth sequence of SEQ ID NO: 1313 linked to a fifth sequence of SEQ ID NO: 3644; a fourth sequence of SEQ ID NO: 1313 linked to a fifth sequence of SEQ ID NO: 39: a fourth sequence of SEQ ID NO: 1361 linked to a fifth sequence of SEQ ID NO: 3644: a fourth sequence of SEQ ID NO: 1361 linked to a fifth sequence of SEQ ID NO: 39; a fourth sequence of SEQ ID NO: 1367 linked to a fifth sequence of SEQ ID NO: 3644; a fourth sequence of SEQ ID NO: 1367 linked to a fifth sequence of SEQ ID NO: 39: a fourth sequence of SEQ ID NO: 1367 linked to a fifth sequence of SEQ ID NO: 3644: a fourth sequence of SEQ ID NO: 1367 linked to a fifth sequence of SEQ ID NO: 39; a fourth sequence of SEQ ID NO: 3279 linked to a fifth sequence of SEQ ID NO: 3644; or a fourth sequence of SEQ ID NO: 3279 linked to a fifth sequence of SEQ ID NO: 39.

In some embodiments, the third polypeptide chain, the fourth polypeptide chain, or a combination thereof further comprises the third sequence, wherein the third sequence is linked to the fourth sequence, the fifth sequence, or a combination thereof.

In some embodiments, the third sequence is linked to the N-terminus of the fourth sequence.

In some embodiments, the third sequence is linked to the C-terminus of the fifth sequence.

In some embodiments, the first polypeptide chain comprises: a first sequence of SEQ ID NO: 1 linked to a second sequence of SEQ ID NO: 40; a first sequence of SEQ ID NO: 1 linked to a second sequence of SEQ ID NO: 42; a first sequence of SEQ ID NO: 1 linked to a second sequence of SEQ ID NO: 74; a first sequence of SEQ ID NO: 1 linked to a second sequence of SEQ ID NO: 3645; a first sequence of SEQ ID NO: 1 linked to a second sequence of SEQ ID NO: 3646; a first sequence of SEQ ID NO: 1 linked to a second sequence of SEQ ID NO: 3648; a first sequence of SEQ ID NO: 1 linked to a second sequence of SEQ ID NO: 3649; a first sequence of SEQ ID NO: 9 linked to a second sequence of SEQ ID NO: 40; a first sequence of SEQ ID NO: 9 linked to a second sequence of SEQ ID NO: 42; a first sequence of SEQ ID NO: 9 linked to a second sequence of SEQ ID NO: 74; a first sequence of SEQ ID NO: 9 linked to a second sequence of SEQ ID NO: 3645; a first sequence of SEQ ID NO: 9 linked to a second sequence of SEQ ID NO: 3646; a first sequence of SEQ ID NO: 9 linked to a second sequence of SEQ ID NO: 3648; a first sequence of SEQ ID NO: 9 linked to a second sequence of SEQ ID NO: 3649; a first sequence of SEQ ID NO: 25 linked to a second sequence of SEQ ID NO: 40; a first sequence of SEQ ID NO: 25 linked to a second sequence of SEQ ID NO. 42; a first sequence of SEQ ID NO. 25 linked to a second sequence of SEQ ID NO: 74; a first sequence of SEQ ID NO: 25 linked to a second sequence of SEQ ID NO: 3645; a first sequence of SEQ ID NO: 25 linked to a second sequence of SEQ ID NO: 3646; a first sequence of SEQ ID NO: 25 linked to a second sequence of SEQ ID NO: 3648; a first sequence of SEQ ID NO: 25 linked to a second sequence of SEQ ID NO: 3649; a first sequence of SEQ ID NO: 82 linked to a second sequence of SEQ ID NO: 40; a first sequence of SEQ ID NO: 82 linked to a second sequence of SEQ ID NO: 42, a first sequence of SEQ ID NO: 82 linked to a second sequence of SEQ ID NO: 74; a first sequence of SEQ ID NO: 82 linked to a second sequence of SEQ ID NO: 3645; a first sequence of SEQ ID NO: 82 linked to a second sequence of SEQ ID NO: 3646; a first sequence of SEQ ID NO: 82 linked to a second sequence of SEQ ID NO: 3648; a first sequence of SEQ ID NO. 82 linked to a second sequence of SEQ ID NO: 3649; a first sequence of SEQ ID NO: 91 linked to a second sequence of SEQ ID NO: 40; a first sequence of SEQ ID NO: 91 linked to a second sequence of SEQ ID NO: 42; a first sequence of SEQ ID NO: 91 linked to a second sequence of SEQ ID NO: 74; a first sequence of SEQ ID NO: 91 linked to a second sequence of SEQ ID NO: 3645; a first sequence of SEQ ID NO: 91 linked to a second sequence of SEQ ID NO: 3646; a first sequence of SEQ ID NO: 91 linked to a second sequence of SEQ ID NO: 3648; a first sequence of SEQ ID NO: 91 linked to a second sequence of SEQ ID NO: 3649; a first sequence of SEQ ID NO: 103 linked to a second sequence of SEQ ID NO: 40: a first sequence of SEQ ID NO: 103 linked to a second sequence of SEQ ID NO: 42; a first sequence of SEQ ID NO: 103 linked to a second sequence of SEQ ID NO: 74; a first sequence of SEQ ID NO: 103 linked to a second sequence of SEQ ID NO: 3645; a first sequence of SEQ ID NO: 103 linked to a second sequence of SEQ ID NO: 3646; a first sequence of SEQ ID NO: 103 linked to a second sequence of SEQ ID NO: 3648; a first sequence of SEQ ID NO: 103 linked to a second sequence of SEQ ID NO: 3649; a first sequence of SEQ ID NO: 118 linked to a second sequence of SEQ ID NO: 40; a first sequence of SEQ ID NO: 118 linked to a second sequence of SEQ ID NO: 42; a first sequence of SEQ ID NO: 118 linked to a second sequence of SEQ ID NO: 74; a first sequence of SEQ ID NO: 118 linked to a second sequence of SEQ ID NO: 3645; a first sequence of SEQ ID NO: 118 linked to a second sequence of SEQ ID NO: 3646; a first sequence of SEQ ID NO: 118 linked to a second sequence of SEQ ID NO: 3648; a first sequence of SEQ ID NO: 118 linked to a second sequence of SEQ ID NO: 3649; a first sequence of SEQ ID NO: 130 linked to a second sequence of SEQ ID NO: 40; a first sequence of SEQ ID NO: 130 linked to a second sequence of SEQ ID NO: 42; a first sequence of SEQ ID NO. 130 linked to a second sequence of SEQ ID NO: 74; a first sequence of SEQ ID NO: 130 linked to a second sequence of SEQ ID NO: 3645; a first sequence of SEQ ID NO: 130 linked to a second sequence of SEQ ID NO: 3646; a first sequence of SEQ ID NO: 130 linked to a second sequence of SEQ ID NO: 3648; a first sequence of SEQ ID NO: 130 linked to a second sequence of SEQ ID NO: 3649: a first sequence of SEQ ID NO: 142 linked to a second sequence of SEQ ID NO. 40; a first sequence of SEQ ID NO: 142 linked to a second sequence of SEQ ID NO: 42; a first sequence of SEQ ID NO: 142 linked to a second sequence of SEQ ID NO: 74; a first sequence of SEQ ID NO: 142 linked to a second sequence of SEQ ID NO: 3645; a first sequence of SEQ ID NO. 142 linked to a second sequence of SEQ ID NO: 3646; a first sequence of SEQ ID NO: 142 linked to a second sequence of SEQ ID NO: 3648; a first sequence of SEQ ID NO: 142 linked to a second sequence of SEQ ID NO: 3649; a first sequence of SEQ ID NO: 151 linked to a second sequence of SEQ ID NO: 40; a first sequence of SEQ ID NO: 151 linked to a second sequence of SEQ ID NO: 42; a first sequence of SEQ ID NO: 151 linked to a second sequence of SEQ ID NO: 74; a first sequence of SEQ ID NO: 151 linked to a second sequence of SEQ ID NO: 3645; a first sequence of SEQ ID NO: 151 linked to a second sequence of SEQ ID NO: 3646; a first sequence of SEQ ID NO: 151 linked to a second sequence of SEQ ID NO: 3648; a first sequence of SEQ ID NO: 151 linked to a second sequence of SEQ ID NO: 3649; a first sequence of SEQ ID NO: 167 linked to a second sequence of SEQ ID NO. 40; a first sequence of SEQ ID NO: 167 linked to a second sequence of SEQ ID NO: 42; a first sequence of SEQ ID NO: 167 linked to a second sequence of SEQ ID NO: 74; a first sequence of SEQ ID NO: 167 linked to a second sequence of SEQ ID NO: 3645; a first sequence of SEQ ID NO: 167 linked to a second sequence of SEQ ID NO: 3646; a first sequence of SEQ ID NO: 167 linked to a second sequence of SEQ ID NO: 3648: a first sequence of SEQ ID NO: 167 linked to a second sequence of SEQ ID NO: 3649; a first sequence of SEQ ID NO: 182 linked to a second sequence of SEQ ID NO: 40; a first sequence of SEQ ID NO: 182 linked to a second sequence of SEQ ID NO: 42; a first sequence of SEQ ID NO: 182 linked to a second sequence of SEQ ID NO: 74; a first sequence of SEQ ID NO: 182 linked to a second sequence of SEQ ID NO: 3645; a first sequence of SEQ ID NO: 182 linked to a second sequence of SEQ ID NO: 3646; a first sequence of SEQ ID NO: 182 linked to a second sequence of SEQ ID NO: 3648; a first sequence of SEQ ID NO: 182 linked to a second sequence of SEQ ID NO: 3649; a first sequence of SEQ ID NO: 197 linked to a second sequence of SEQ ID NO: 40; a first sequence of SEQ ID NO: 197 linked to a second sequence of SEQ ID NO: 42; a first sequence of SEQ ID NO: 197 linked to a second sequence of SEQ ID NO: 74; a first sequence of SEQ ID NO: 197 linked to a second sequence of SEQ ID NO: 3645; a first sequence of SEQ ID NO: 197 linked to a second sequence of SEQ ID NO: 3646; a first sequence of SEQ ID NO: 197 linked to a second sequence of SEQ ID NO: 3648; a first sequence of SEQ ID NO: 197 linked to a second sequence of SEQ ID NO: 3649; a first sequence of SEQ ID NO: 203 linked to a second sequence of SEQ ID NO: 40; a first sequence of SEQ ID NO: 203 linked to a second sequence of SEQ ID NO: 42; a first sequence of SEQ ID NO: 203 linked to a second sequence of SEQ ID NO: 74; a first sequence of SEQ ID NO: 203 linked to a second sequence of SEQ ID NO: 3645; a first sequence of SEQ ID NO: 203 linked to a second sequence of SEQ ID NO: 3646; a first sequence of SEQ ID NO: 203 linked to a second sequence of SEQ ID NO: 3648; a first sequence of SEQ ID NO: 203 linked to a second sequence of SEQ ID NO: 3649; a first sequence of SEQ ID NO: 209 linked to a second sequence of SEQ ID NO: 40; a first sequence of SEQ ID NO. 209 linked to a second sequence of SEQ ID NO: 42; a first sequence of SEQ ID NO: 209 linked to a second sequence of SEQ ID NO: 74; a first sequence of SEQ ID NO. 209 linked to a second sequence of SEQ ID NO: 3645; a first sequence of SEQ ID NO: 209 linked to a second sequence of SEQ ID NO: 3646; a first sequence of SEQ ID NO. 209 linked to a second sequence of SEQ ID NO: 3648; a first sequence of SEQ ID NO: 209 linked to a second sequence of SEQ ID NO: 3649; a first sequence of SEQ ID NO: 215 linked to a second sequence of SEQ ID NO: 40: a first sequence of SEQ ID NO: 215 linked to a second sequence of SEQ ID NO: 42; a first sequence of SEQ ID NO: 215 linked to a second sequence of SEQ ID NO: 74; a first sequence of SEQ ID NO: 215 linked to a second sequence of SEQ ID NO: 3645; a first sequence of SEQ ID NO: 215 linked to a second sequence of SEQ ID NO: 3646; a first sequence of SEQ ID NO: 215 linked to a second sequence of SEQ ID NO: 3648; a first sequence of SEQ ID NO: 215 linked to a second sequence of SEQ ID NO: 3649; a first sequence of SEQ ID NO: 221 linked to a second sequence of SEQ ID NO: 40; a first sequence of SEQ ID NO: 221 linked to a second sequence of SEQ ID NO: 42; a first sequence of SEQ ID NO: 221 linked to a second sequence of SEQ ID NO: 74; a first sequence of SEQ ID NO: 221 linked to a second sequence of SEQ ID NO: 3645; a first sequence of SEQ ID NO: 221 linked to a second sequence of SEQ ID NO: 3646; a first sequence of SEQ ID NO: 221 linked to a second sequence of SEQ ID NO: 3648; a first sequence of SEQ ID NO: 221 linked to a second sequence of SEQ ID NO: 3649; a first sequence of SEQ ID NO: 1100 linked to a second sequence of SEQ ID NO: 40; a first sequence of SEQ ID NO: 1100 linked to a second sequence of SEQ ID NO: 42; a first sequence of SEQ ID NO: 1100 linked to a second sequence of SEQ ID NO: 74; a first sequence of SEQ ID NO: 1100 linked to a second sequence of SEQ ID NO: 3645; a first sequence of SEQ ID NO: 1100 linked to a second sequence of SEQ ID NO: 3646; a first sequence of SEQ ID NO: 1100 linked to a second sequence of SEQ ID NO: 3648; a first sequence of SEQ ID NO: 1100 linked to a second sequence of SEQ ID NO: 3649; a first sequence of SEQ ID NO: 1310 linked to a second sequence of SEQ ID NO: 40; a first sequence of SEQ ID NO: 1310 linked to a second sequence of SEQ ID NO: 42; a first sequence of SEQ ID NO: 1310 linked to a second sequence of SEQ ID NO: 74; a first sequence of SEQ ID NO: 1310 linked to a second sequence of SEQ ID NO: 3645; a first sequence of SEQ ID NO: 1310 linked to a second sequence of SEQ ID NO: 3646; a first sequence of SEQ ID NO: 1310 linked to a second sequence of SEQ ID NO: 3648; a first sequence of SEQ ID NO: 1310 linked to a second sequence of SEQ ID NO: 3649; a first sequence of SEQ ID NO: 1346 linked to a second sequence of SEQ ID NO. 40; a first sequence of SEQ ID NO. 1346 linked to a second sequence of SEQ ID NO: 42; a first sequence of SEQ ID NO: 1346 linked to a second sequence of SEQ ID NO: 74; a first sequence of SEQ ID NO: 1346 linked to a second sequence of SEQ ID NO: 3645; a first sequence of SEQ ID NO: 1346 linked to a second sequence of SEQ ID NO: 3646; a first sequence of SEQ ID NO: 1346 linked to a second sequence of SEQ ID NO: 3648; a first sequence of SEQ ID NO: 1346 linked to a second sequence of SEQ ID NO: 3649; a first sequence of SEQ ID NO: 1350 linked to a second sequence of SEQ ID NO: 40; a first sequence of SEQ ID NO. 1350 linked to a second sequence of SEQ ID NO: 42; a first sequence of SEQ ID NO: 1350 linked to a second sequence of SEQ ID NO: 74; a first sequence of SEQ ID NO: 1350 linked to a second sequence of SEQ ID NO: 3645; a first sequence of SEQ ID NO. 1350 linked to a second sequence of SEQ ID NO: 3646; a first sequence of SEQ ID NO: 1350 linked to a second sequence of SEQ ID NO: 3648; a first sequence of SEQ ID NO: 1350 linked to a second sequence of SEQ ID NO: 3649; a first sequence of SEQ ID NO: 1360 linked to a second sequence of SEQ ID NO: 40; a first sequence of SEQ ID NO: 1360 linked to a second sequence of SEQ ID NO: 42; a first sequence of SEQ ID NO: 1360 linked to a second sequence of SEQ ID NO: 74; a first sequence of SEQ ID NO. 1360 linked to a second sequence of SEQ ID NO: 3645; a first sequence of SEQ ID NO: 1360 linked to a second sequence of SEQ ID NO: 3646; a first sequence of SEQ ID NO: 1360 linked to a second sequence of SEQ ID NO: 3648; a first sequence of SEQ ID NO: 1360 linked to a second sequence of SEQ ID NO: 3649; a first sequence of SEQ ID NO: 1370 linked to a second sequence of SEQ ID NO. 40; a first sequence of SEQ ID NO. 1370 linked to a second sequence of SEQ ID NO: 42; a first sequence of SEQ ID NO: 1370 linked to a second sequence of SEQ ID NO: 74; a first sequence of SEQ ID NO: 1370 linked to a second sequence of SEQ ID NO: 3645; a first sequence of SEQ ID NO: 1370 linked to a second sequence of SEQ ID NO: 3646; a first sequence of SEQ ID NO: 1370 linked to a second sequence of SEQ ID NO: 3648; or a first sequence of SEQ ID NO: 1370 linked to a second sequence of SEQ ID NO: 3649.

In some embodiments, the second polypeptide chain comprises the sequence of SEQ ID NO: 2191 linked to the sequence of SEQ ID NO: 40; the sequence of SEQ ID NO: 2191 linked to the sequence of SEQ ID NO: 42; the sequence of SEQ ID NO: 2191 linked to the sequence of SEQ ID NO: 74; the sequence of SEQ ID NO: 2191 linked to the sequence of SEQ ID NO: 3645; the sequence of SEQ ID NO: 2191 linked to the sequence of SEQ ID NO: 3646; the sequence of SEQ ID NO: 2191 linked to the sequence of SEQ ID NO: 3648; the sequence of SEQ ID NO: 2191 linked to the sequence of SEQ ID NO: 3649; the sequence of SEQ ID NO: 2270 linked to the sequence of SEQ ID NO: 40; the sequence of SEQ ID NO: 2270 linked to the sequence of SEQ ID NO: 42; the sequence of SEQ ID NO: 2270 linked to the sequence of SEQ ID NO: 74; the sequence of SEQ ID NO. 2270 linked to the sequence of SEQ ID NO: 3645; the sequence of SEQ ID NO: 2270 linked to the sequence of SEQ ID NO: 3646; the sequence of SEQ ID NO: 2270 linked to the sequence of SEQ ID NO: 3648; or the sequence of SEQ ID NO: 2270 linked to the sequence of SEQ ID NO: 3649.

In some embodiments, the third polypeptide chain comprises: a fourth sequence of SEQ ID NO: 2 linked to a fifth sequence of SEQ ID NO: 3644; a fourth sequence of SEQ ID NO: 2 linked to a fifth sequence of SEQ ID NO: 39; a fourth sequence of SEQ ID NO: 10 linked to a fifth sequence of SEQ ID NO: 3644; a fourth sequence of SEQ ID NO: 10 linked to a fifth sequence of SEQ ID NO: 39; a fourth sequence of SEQ ID NO: 16 linked to a fifth sequence of SEQ ID NO: 3644, a fourth sequence of SEQ ID NO. 16 linked to a fifth sequence of SEQ ID NO: 39; a fourth sequence of SEQ ID NO: 28 linked to a fifth sequence of SEQ ID NO: 3644; a fourth sequence of SEQ ID NO: 28 linked to a fifth sequence of SEQ ID NO: 39; a fourth sequence of SEQ ID NO: 87 linked to a fifth sequence of SEQ ID NO: 3644; a fourth sequence of SEQ ID NO: 87 linked to a fifth sequence of SEQ ID NO: 39: a fourth sequence of SEQ ID NO: 90 linked to a fifth sequence of SEQ ID NO. 3644; a fourth sequence of SEQ ID NO: 90 linked to a fifth sequence of SEQ ID NO: 39; a fourth sequence of SEQ ID NO: 96 linked to a fifth sequence of SEQ ID NO: 3644: a fourth sequence of SEQ ID NO: 96 linked to a fifth sequence of SEQ ID NO: 39: a fourth sequence of SEQ ID NO: 105 linked to a fifth sequence of SEQ ID NO: 3644: a fourth sequence of SEQ ID NO: 105 linked to a fifth sequence of SEQ ID NO: 39: a fourth sequence of SEQ ID NO: 117 linked to a fifth sequence of SEQ ID NO. 3644; a fourth sequence of SEQ ID NO: 117 linked to a fifth sequence of SEQ ID NO: 39: a fourth sequence of SEQ ID NO: 120 linked to a fifth sequence of SEQ ID NO: 3644: a fourth sequence of SEQ ID NO: 120 linked to a fifth sequence of SEQ ID NO: 39: a fourth sequence of SEQ ID NO: 129 linked to a fifth sequence of SEQ ID NO: 3644: a fourth sequence of SEQ ID NO: 129 linked to a fifth sequence of SEQ ID NO: 39; a fourth sequence of SEQ ID NO: 132 linked to a fifth sequence of SEQ ID NO: 3644: a fourth sequence of SEQ ID NO: 132 linked to a fifth sequence of SEQ ID NO: 39; a fourth sequence of SEQ ID NO: 141 linked to a fifth sequence of SEQ ID NO: 3644: a fourth sequence of SEQ ID NO: 141 linked to a fifth sequence of SEQ ID NO: 39: a fourth sequence of SEQ ID NO: 150 linked to a fifth sequence of SEQ ID NO: 3644; a fourth sequence of SEQ ID NO: 150 linked to a fifth sequence of SEQ ID NO: 39; a fourth sequence of SEQ ID NO: 154 linked to a fifth sequence of SEQ ID NO: 3644; a fourth sequence of SEQ ID NO: 154 linked to a fifth sequence of SEQ ID NO: 39: a fourth sequence of SEQ ID NO: 163 linked to a fifth sequence of SEQ ID NO: 3644; a fourth sequence of SEQ ID NO: 163 linked to a fifth sequence of SEQ ID NO: 39: a fourth sequence of SEQ ID NO: 169 linked to a fifth sequence of SEQ ID NO: 3644; a fourth sequence of SEQ ID NO: 169 linked to a fifth sequence of SEQ ID NO: 39: a fourth sequence of SEQ ID NO: 175 linked to a fifth sequence of SEQ ID NO: 3644; a fourth sequence of SEQ ID NO: 175 linked to a fifth sequence of SEQ ID NO: 39; a fourth sequence of SEQ ID NO: 181 linked to a fifth sequence of SEQ ID NO: 3644: a fourth sequence of SEQ ID NO: 181 linked to a fifth sequence of SEQ ID NO: 39: a fourth sequence of SEQ ID NO: 187 linked to a fifth sequence of SEQ ID NO: 3644: a fourth sequence of SEQ ID NO: 187 linked to a fifth sequence of SEQ ID NO: 39: a fourth sequence of SEQ ID NO: 193 linked to a fifth sequence of SEQ ID NO 3644; a fourth sequence of SEQ ID NO: 193 linked to a fifth sequence of SEQ ID NO: 39; a fourth sequence of SEQ ID NO: 202 linked to a fifth sequence of SEQ ID NO: 3644; a fourth sequence of SEQ ID NO: 202 linked to a fifth sequence of SEQ ID NO: 39; a fourth sequence of SEQ ID NO: 1101 linked to a fifth sequence of SEQ ID NO 3644; a fourth sequence of SEQ ID NO: 1101 linked to a fifth sequence of SEQ ID NO: 39: a fourth sequence of SEQ ID NO: 1349 linked to a fifth sequence of SEQ ID NO: 3644; a fourth sequence of SEQ ID NO: 1349 linked to a fifth sequence of SEQ ID NO: 39; a fourth sequence of SEQ ID NO: 1313 linked to a fifth sequence of SEQ ID NO: 3644: a fourth sequence of SEQ ID NO: 1313 linked to a fifth sequence of SEQ ID NO: 39; a fourth sequence of SEQ ID NO: 1361 linked to a fifth sequence of SEQ ID NO: 3644: a fourth sequence of SEQ ID NO: 1361 linked to a fifth sequence of SEQ ID NO: 39; a fourth sequence of SEQ ID NO. 1367 linked to a fifth sequence of SEQ ID NO: 3644: a fourth sequence of SEQ ID NO: 1367 linked to a fifth sequence of SEQ ID NO: 39: a fourth sequence of SEQ ID NO. 1367 linked to a fifth sequence of SEQ ID NO: 3644: a fourth sequence of SEQ ID NO. 1367 linked to a fifth sequence of SEQ ID NO: 39: a fourth sequence of SEQ ID NO: 3279 linked to a fifth sequence of SEQ ID NO: 3644; or a fourth sequence of SEQ ID NO: 3279 linked to a fifth sequence of SEQ ID NO: 39.

In some embodiments, the multifunctional polypeptide molecule comprises: (i) a first polypeptide chain comprising an anti-TCRvβ antibody heavy chain variable region, and an immunoglobulin heavy chain constant region; (ii) a second polypeptide chain comprising an IL-15 receptor alpha sushi domain or a functional fragment or a functional variant thereof, an IL-15 molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-15 receptor alpha sushi domain or a functional fragment or a functional variant thereof operatively linked to an IL-15 molecule or a functional fragment or a functional variant thereof operatively linked to an immunoglobulin heavy chain constant region; and (iii) a third polypeptide chain comprising, from the N-terminus to the C-terminus, an anti-TCRvβ antibody light chain variable region operatively linked to an immunoglobulin light chain constant region.

In some embodiments, the IL-15 receptor alpha sushi domain is operatively linked to the IL-15 molecule or a functional fragment or a functional variant thereof via a linker, the IL-15 molecule or a functional fragment or a functional variant thereof is operatively linked to the immunoglobulin heavy chain constant region via a linker, or a combination thereof.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO. 3523, the sequence of SEQ ID NO: 2170, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 3523 operatively linked to the sequence of SEQ ID NO: 2170 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the sequence of SEQ ID NO: 3523 is operatively linked to the sequence of SEQ ID NO: 2170 via the sequence of SEQ ID NO: 3524, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3519; and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO. 3517; (ii) a second polypeptide chain comprising the sequence of SEQ ID NO: 3519; and (iii) a third polypeptide chain comprising to the sequence of SEQ ID NO: 3518.

In some embodiments, the multifunctional polypeptide molecule comprises: (i) a first polypeptide chain comprising an anti-TCRvβ antibody heavy chain variable region, and an immunoglobulin heavy chain constant region; (ii) a second polypeptide chain comprising an IL-15 molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-15 molecule or a functional fragment or a functional variant thereof operatively linked to an immunoglobulin heavy chain constant region; and (iii) a third polypeptide chain comprising, from the N-terminus to the C-terminus, an anti-TCRvβ antibody light chain variable region operatively linked to an immunoglobulin light chain constant region.

In some embodiments, the IL-15 molecule or a functional fragment or a functional variant thereof is operatively linked to the immunoglobulin heavy chain constant region via a linker.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 2170, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 2170 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3520; and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3520; and (iii) a third polypeptide chain comprising to the sequence of SEQ ID NO: 3518.

In some embodiments, the multifunctional polypeptide molecule comprises: (i) a first polypeptide chain comprising an anti-TCRvβ antibody heavy chain variable region, and an immunoglobulin heavy chain constant region; (ii) a second polypeptide chain comprising an IL-2 molecule or a functional fragment or a functional variant thereof or an IL-2 C125A mutant molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-2 molecule or a functional fragment or a functional variant thereof or an IL-2 C125A mutant molecule or a functional fragment or a functional variant thereof operatively linked to an immunoglobulin heavy chain constant region; and (iii) a third polypeptide chain comprising, from the N-terminus to the C-tenninus, an anti-TCRvβ antibody light chain variable region operatively linked to an immunoglobulin light chain constant region.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 2270, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 2270 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the sequence of SEQ ID NO: 2270 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3521; and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3521; and (iii) a third polypeptide chain comprising to the sequence of SEQ ID NO: 3518.

In some embodiments, the multifunctional poly peptide molecule comprises the second polypeptide chain comprising an immunoglobulin heavy chain constant region comprising L234A, L235A, and P329G mutations, the third polypeptide chain comprising an immunoglobulin light chain constant region comprising L234A, L235A, and P329G mutations, or a combination thereof.

In some embodiments, the multifunctional polypeptide molecule comprises: (i) a first polypeptide chain comprising the sequence of SEQ ID NO: 3530, and the sequence of SEQ ID NO: 3531; (ii) a second polypeptide chain comprising the sequence of SEQ ID NO: 2191, and the sequence of SEQ ID NO: 3533; and (iii) a third polypeptide chain comprising the sequence of SEQ ID NO: 3527, and the sequence of SEQ ID NO: 3528.

In some embodiments, the multifunctional poly peptide molecule comprises: (i) a first polypeptide chain comprising, from the N-terminus to the C-terminus, the sequence of SEQ ID NO: 3530 operatively linked to the sequence of SEQ ID NO: 3531; (ii) a second polypeptide chain comprising, from the N-terminus to the C-terminus, the sequence of SEQ ID NO: 2191 operatively linked to the sequence of SEQ ID NO: 3533; and (iii) a third polypeptide chain comprising, from the N-terminus to the C-terminus, the sequence of SEQ ID NO: 3527 operatively linked to the sequence of SEQ ID NO: 3528.

In some embodiments, the sequence of SEQ ID NO: 2191 is operatively linked to the sequence of SEQ ID NO: 3533 via the sequence of SEQ ID NO: 3308, or a combination thereof.

In some embodiments, the first polypeptide chain further comprises the sequence of SEQ ID NO. 3547 operatively linked to the sequence of SEQ ID NO: 3531, the second polypeptide chain further comprises the sequence of SEQ ID NO: 3534 operatively linked to the sequence of SEQ ID NO: 3533, or a combination thereof.

In some embodiments, the multifunctional polypeptide molecule comprises: (i) a first polypeptide chain comprising a sequence having at least 75% sequence identity to the sequence of SEQ ID NO: 3529 or the sequence of SEQ ID NO: 3548; (ii) a second polypeptide chain comprising a sequence having at least 75% sequence identity to the sequence of SEQ ID NO: 3532 or the sequence of SEQ ID NO: 3549; and (iii) a third polypeptide chain comprising a sequence having at least 75% sequence identity to the sequence of SEQ ID NO: 3526.

In some embodiments, the multifunctional polypeptide molecule comprises: (i) a first polypeptide chain comprising the sequence of SEQ ID NO: 3529 or the sequence of SEQ ID NO: 3548; (ii) a second polypeptide chain comprising the sequence of SEQ ID NO: 3532 or the sequence of SEQ ID NO: 3549; and (ii) a third polypeptide chain comprising to the sequence of SEQ ID NO: 3526.

In some embodiments, the multifunctional polypeptide molecule comprises: (i) a first polypeptide chain comprising an anti-TCRvβ antibody heavy chain variable region, and an immunoglobulin heavy chain constant region; (ii) a second polypeptide chain comprising an IL-7 molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-7 molecule or a functional fragment or a functional variant thereof operatively linked to an immunoglobulin heavy chain constant region; and (iii) a third polypeptide chain comprising, from the N-terminus to the C-terminus, an anti-TCRvβ antibody light chain variable region operatively linked to an immunoglobulin light chain constant region.

In some embodiments, the IL-7 molecule or a functional fragment or a functional variant thereof is operatively linked to the immunoglobulin heavy chain constant region via a linker.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 3540, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 3540 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the sequence of SEQ ID NO: 3540 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3539; and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3539; and (iii) a third polypeptide chain comprising to the sequence of SEQ ID NO: 3518.

In some embodiments, the multifunctional poly peptide molecule comprises: (i) a first polypeptide chain comprising an anti-TCRvβ antibody heavy chain variable region, and an immunoglobulin heavy chain constant region; (ii) a second polypeptide chain comprising an IL-12 molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-12 molecule or a functional fragment or a functional variant thereof operatively linked to an immunoglobulin heavy chain constant region; and (iii) a third polypeptide chain comprising, from the N-terminus to the C-terminus, an anti-TCRvβ antibody light chain variable region operatively linked to an immunoglobulin light chain constant region.

In some embodiments, the IL-12 molecule or a functional fragment or a functional variant thereof comprises an IL-12 beta subunit or a functional fragment or a functional variant thereof and a IL-12 alpha subunit or a functional fragment or a functional variant thereof.

In some embodiments, the IL-12 molecule or a functional fragment or a functional variant thereof comprises, from the N-terminus to the C-terminus, an IL-12 beta subunit or a functional fragment or a functional variant thereof operatively linked to a IL-12 alpha subunit or a functional fragment or a functional variant thereof.

In some embodiments, the IL-12 beta subunit or a functional fragment or a functional variant thereof is operatively linked to the IL-12 alpha subunit or a functional fragment or a functional variant thereof via a linker, the IL-12 alpha subunit or a functional fragment or a functional variant thereof is operatively linked to the immunoglobulin heavy chain constant region via a linker, or a combination thereof.

In some embodiments, the multifunctional polypeptide molecule comprises: (i) a first polypeptide chain comprising the sequence of SEQ ID NO. 1346, and the sequence of SEQ ID NO: 3649; (ii) a second polypeptide chain comprising the sequence of SEQ ID NO: 3542, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 3542 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the IL-12 molecule or a functional fragment or a functional variant thereof comprises the sequence of SEQ ID NO: 3543 and the sequence of SEQ ID NO: 3545.

In some embodiments, the IL-12 molecule or a functional fragment or a functional variant thereof comprises, from the N-terminus to the C-terminus, the sequence of SEQ ID NO: 3543 operatively linked to the sequence of SEQ ID NO:3545.

In some embodiments, the sequence of SEQ ID NO: 3543 is operatively linked to the sequence of SEQ ID NO: 3545 via the sequence of SEQ ID NO: 3544, the sequence of SEQ ID NO: 3545 is operatively linked to the sequence of SEQ ID NO: 3648 via the sequence of SEQ ID NO: 3308, or a combination thereof.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3541, and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3541; and (iii) a third polypeptide chain comprising to the sequence of SEQ ID NO: 3518.

In some embodiments, the multifunctional polypeptide molecule comprises: (i) a first polypeptide chain comprising an anti-TCRvβ antibody heavy chain variable region, and an immunoglobulin heavy chain constant region; (ii) a second polypeptide chain comprising an IL-21 molecule or a functional fragment or a functional variant thereof, and an immunoglobulin heavy chain constant region; and (iii) a third polypeptide chain comprising an anti-TCRvβ antibody light chain variable region, and an immunoglobulin light chain constant region.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; (ii) a second polypeptide chain comprising, from the N-terminus to the C-terminus, an IL-21 molecule or a functional fragment or a functional variant thereof operatively linked to an immunoglobulin heavy chain constant region; and (iii) a third polypeptide chain comprising, from the N-terminus to the C-terminus, an anti-TCRyβ antibody light chain variable region operatively linked to an immunoglobulin light chain constant region.

In some embodiments, the IL-21 molecule or a functional fragment or a functional variant thereof is operatively linked to the immunoglobulin heavy chain constant region via a linker, or a combination thereof.

In some embodiments, the multifunctional polypeptide molecule comprises: (i) a first polypeptide chain comprising the sequence of SEQ ID NO: 1346, and the sequence of SEQ ID NO: 3649; (ii) a second polypeptide chain comprising the sequence of SEQ ID NO: 2193, and the sequence of SEQ ID NO: 3648; and (iii) a third polypeptide chain comprising the sequence of SEQ ID NO: 1349, and the sequence of SEQ ID NO: 3644.

In some embodiments, the multifunctional polypeptide molecule comprises: (i) a first polypeptide chain comprising, from the N-terminus to the C-terminus, the sequence of SEQ ID NO: 1346 operatively linked to the sequence of SEQ ID NO: 3649; (ii) a second polypeptide chain comprising, from the N-terminus to the C-terminus, the sequence of SEQ ID NO: 2193 operatively linked to the sequence of SEQ ID NO: 3648; and (iii) a third polypeptide chain comprising, from the N-terminus to the C-terminus, the sequence of SEQ ID NO: 1349 operatively linked to the sequence of SEQ ID NO: 3644.

In some embodiments, the sequence of SEQ ID NO: 2193 is operatively linked to the sequence of SEQ ID NO: 3648 via the sequence of SEQ ID NO: 3308.

In some embodiments, the multifunctional polypeptide molecule comprises: (i) a first polypeptide chain comprising a sequence having at least 75% sequence identity to the sequence of SEQ ID NO: 3517; (ii) a second polypeptide chain comprising a sequence having at least 75% sequence identity to the sequence of SEQ ID NO: 3546; and (iii) a third polypeptide chain comprising a sequence having at least 75% sequence identity to the sequence of SEQ ID NO: 3518.

In some embodiments, the multifunctional polypeptide molecule comprises: (i) a first polypeptide chain comprising the sequence of SEQ ID NO: 3517; (ii) a second polypeptide chain comprising the sequence of SEQ ID NO: 3546; and (iii) a third polypeptide chain comprising to the sequence of SEQ ID NO: 3518.

In some embodiments, the multifunctional polypeptide molecule comprises: (i) a first polypeptide chain comprising an anti-TCRvβ antibody heavy chain variable region, and an immunoglobulin heavy chain constant region; and (ii) a second poly peptide chain comprising an anti-TCRv antibody light chain variable region, an immunoglobulin light chain constant region, and an IL-2 molecule or a functional fragment or a functional variant thereof.

In some embodiments, the multifunctional polypeptide molecule comprises: (i) a first polypeptide chain comprising, from the N-terminus to the C-terminus, an anti-TCRvβ antibody heavy chain variable region operatively linked to an immunoglobulin heavy chain constant region; and (ii) a second polypeptide chain comprising, from the N-terminus to the C-terminus, an anti-TCRvβ antibody light chain variable region operatively linked to an immunoglobulin light chain constant region operatively linked to an IL-2 molecule or a functional fragment or a functional variant thereof.

In some embodiments, the immunoglobulin light chain constant region is operatively linked to the IL-21 molecule or a functional fragment or a functional variant thereof via a linker.

In some embodiments, the multifunctional polypeptide molecule comprises two first polypeptide chains and two second polypeptide chains.

In some embodiments, the multifunctional polypeptide molecule comprises the first polypeptide chain comprising an immunoglobulin heavy chain constant region comprising L234A, L235A, and P329G mutations.

In some embodiments, the multifunctional polypeptide molecule comprises: (i) a first polypeptide chain comprising the sequence of SEQ ID NO: 3530 and the sequence of SEQ ID NO: 3537; and (ii) a second polypeptide chain comprising the sequence of SEQ ID NO: 3527, the sequence of SEQ ID NO: 3528, and the sequence of SEQ ID NO: 2191.

In some embodiments, the multifunctional polypeptide molecule comprises: (i) a first polypeptide chain comprising, from the N-terminus to the C-terminus, the sequence of SEQ ID NO: 3530 operatively linked to the sequence of SEQ ID NO: 3537; and (ii) a second polypeptide chain comprising, from the N-terminus to the C-terminus, the sequence of SEQ ID NO: 3527 operatively linked to the sequence of SEQ ID NO: 3528 operatively linked to the sequence of SEQ ID NO: 2191.

In some embodiments, the sequence of SEQ ID NO: 3528 is operatively linked to the sequence of SEQ ID NO: 2191 via the sequence of SEQ ID NO: 3309.

In some embodiments, the multifunctional polypeptide molecule comprises two first polypeptide chains and two second polypeptide chains.

In some embodiments, the multifunctional polypeptide molecule comprises: (i) a first polypeptide chain comprising a sequence having at least 75% sequence identity to the sequence of SEQ ID NO: 3536; and (ii) a second polypeptide chain comprising a sequence having at least 75% sequence identity to the sequence of SEQ ID NO: 3535.

In some embodiments, the multifunctional polypeptide molecule comprises: (i) a first polypeptide chain comprising the sequence of SEQ ID NO. 3536; and (ii) a second polypeptide chain comprising the sequence of SEQ ID NO: 3535.

In another aspect, described herein is an antibody comprising an anti-T cell receptor beta variable chain (TCRβV) binding domain comprising: (i) a heavy chain variable region (VH) comprising a heavy chain complementarity determining region 1 (HC CDR1), a heavy chain complementarity determining region 2 (HC CDR2), and a heavy chain complementarity determining region 3 (HC CDR3) comprising an amino acid sequence having at least 75% sequence identity to SEQ ID NO: 3650, SEQ ID NO: 3651, and SEQ ID NO: 5, respectively; (ii) a light chain variable region (VL) comprising a light chain complementarity determining region 1 (LC CDR1), a light chain complementarity determining region 2 (LC CDR2), and a light chain complementarity determining region 3 (LC CDR3) comprising an amino acid sequence having at least 75% sequence identity to SEQ ID NO: 3655, SEQ ID NO: 3653, and SEQ ID NO: 8, respectively; or (iii) a combination thereof.

In some embodiments, the TCRβV binding domain comprising: (i) a VH comprising a HC CDR1, a HC CDR2, and a HC CDR3 comprising the amino acid sequence of SEQ ID NO: 3650, SEQ ID NO: 3651, and SEQ ID NO: 5, respectively; (ii) a VL comprising a LC CDR1, a LC CDR2, and a LC CDR3 comprising the amino acid sequence of SEQ ID NO: 3655, SEQ ID NO: 3653, and SEQ ID NO: 8, respectively; or (iii) a combination thereof.

In some embodiments, the TCRβV binding domain comprising: (i) a VH comprising an amino acid sequence having at least 75% sequence identity to SEQ ID NO: 1346; (ii) a VL comprising an amino acid sequence having at least 75% sequence identity to SEQ ID NO: 1349; or (iii) a combination thereof.

In some embodiments, the TCRβV binding domain comprising: (i) a VH comprising the amino acid sequence of SEQ ID NO. 1346; (ii) a VL comprising the amino acid sequence of SEQ ID NO: 1349; or (iii) a combination thereof.

In an aspect, described herein is a nucleic acid molecule comprising a nucleotide sequence encoding the multifunctional polypeptide molecule as described herein or the antibody as described herein.

In some embodiments, the nucleic acid molecule is an isolated nucleic acid molecule.

In an aspect, described herein is a vector comprising one or more of the nucleic acid molecules as described herein.

In an aspect, described herein is a cell comprising the nucleic acid molecules as described herein, or the vector as described herein.

In an aspect, described herein is a pharmaceutical composition comprising the multifunctional polypeptide molecule as described herein, the antibody as described herein, the nucleic acid molecules as described herein, the vector as described herein, or the cell as described herein, and a pharmaceutically acceptable carrier, excipient, or diluent.

In an aspect, described herein is a method of treating a condition or disease in a subject in need therefor comprising administering to the subject a therapeutically effective amount of the multifunctional polypeptide molecule as described herein, the antibody as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer.

In some embodiments, the cancer is a solid tumor, a hematological cancer, a metastatic cancer, a soft tissue tumor, or a combination thereof.

In some embodiments, the cancer is the solid tumor, and the solid tumor is selected from the group consisting of melanoma, pancreatic cancer, breast cancer, colorectal cancer, lung cancer, skin cancer, ovarian cancer, liver cancer, and a combination thereof.

In some embodiments, the cancer is the hematological cancer, and the hematological cancer is selected from the group consisting of Hodgkin's lymphoma. Non-Hodgkin's lymphoma, acute myeloid leukemia (AML), chronic myeloid leukemia, myelodysplastic syndrome, multiple myeloma. T-cell lymphoma, acute lymphocytic leukemia, and a combination thereof.

In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof.

In some embodiments, the T-cell lymphoma is peripheral T-cell lymphoma.

In some embodiments, the cancer is characterized by a cancer antigen present on the cancer.

In some embodiments, the cancer antigen is a tumor antigen, a stromal antigen, or a hematological antigen.

In some embodiments, the cancer antigen is selected from the group consisting of BCMA, CD19, CD20, CD22. FcRH5, PDL1. CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7. Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1. CA-125, BAGE, GAGE, NY-ESO-1, β-catenin. CDK4, CDC27, a actinin-4, TRP 1/gp75. TRP2, gp 100, Melan-A/MARTI, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Integrins, carbohydrates, IGF1R, EPHA3, TRAILR1, TRAILR2, RANKL, FAP, TGF-beta, hyaluronic acid, collagen, tenascin C, and tenascin W.

In some embodiments, the method further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the antibody as described herein, the nucleic acid molecules as described herein, the vector as described herein, the cell as described herein, the pharmaceutical composition as described herein, sequentially, simultaneously, or concurrently.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 and a sequence having at least 50%, 55%, 60%, 65%, 70%, 75%, 80%, 85%, 90%, 95%, 96%, 97%, 98%, 99%, 99.5%. 99.7%, or 99.9% sequence identity to the sequence of SEQ ID NO: 3792; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 and a sequence having at least 50%, 55%, 60%, 65%, 70%, 75%, 80%, 85%, 90%, 95%, 96%, 97%, 98%, 99%, 99.5%. 99.7%, or 99.9% sequence identity to the sequence of SEQ ID NO: 3794; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3792; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3794; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3794 via the sequence of SEQ ID NO: 3308.

In some embodiments, the multifunctional molecule comprises: (i) a first polypeptide chain comprising the sequence of SEQ ID NO: 1346 and the sequence of SEQ ID NO: 3792; (ii) a second polypeptide chain comprising the sequence of SEQ ID NO: 2270 and the sequence of SEQ ID NO: 3794; (iii) a third polypeptide chain comprising the sequence of SEQ ID NO: 1349 and the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising the sequence of SEQ ID NO: 1346 operatively linked to the sequence of SEQ ID NO: 3792; (ii) a second polypeptide chain comprising the sequence of SEQ ID NO: 2270 operatively linked to the sequence of SEQ ID NO: 3794; (iii) a third polypeptide chain comprising the sequence of SEQ ID NO: 1349 operatively linked to the sequence of SEQ ID NO: 3644, or (iv) a combination thereof. In some embodiments, the second polypeptide chain comprises the sequence of SEQ ID NO: 2270 operatively linked to the sequence of SEQ ID NO: 3794 via the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3791; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3793; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3518, or (iv) a combination thereof.

In some embodiments, the multifunctional molecule comprises: (i) a first polypeptide chain comprising the sequence of SEQ ID NO: 3791; (ii) a second polypeptide chain comprising the sequence of SEQ ID NO: 3793; (iii) a third polypeptide chain comprising a sequence the sequence of SEQ ID NO: 3518, or (iv) a combination thereof.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1113 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3794; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3797 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1113 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3794; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3797 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3794 via the sequence of SEQ ID NO: 3308.

In some embodiments, the multifunctional molecule comprises: (i) a first polypeptide chain comprising the sequence of SEQ ID NO: 1113 and the sequence of SEQ ID NO: 3649; (ii) a second polypeptide chain comprising the sequence of SEQ ID NO: 2270 and the sequence of SEQ ID NO: 3794; (iii) a third polypeptide chain comprising the sequence of SEQ ID NO: 3797 and the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising the sequence of SEQ ID NO: 1113 operatively linked to the sequence of SEQ ID NO: 3649; (ii) a second polypeptide chain comprising the sequence of SEQ ID NO: 2270 operatively linked to the sequence of SEQ ID NO: 3794; (iii) a third polypeptide chain comprising the sequence of SEQ ID NO: 3797 operatively linked to the sequence of SEQ ID NO: 3644, or (iv) a combination thereof. In some embodiments, the second polypeptide chain comprises the sequence of SEQ ID NO: 2270 operatively linked to the sequence of SEQ ID NO: 3794 via the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3795; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3793; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3796, or (iv) a combination thereof.

In some embodiments, the multifunctional molecule comprises: (i) a first polypeptide chain comprising the sequence of SEQ ID NO: 3791; (ii) a second polypeptide chain comprising the sequence of SEQ ID NO: 3795; (iii) a third polypeptide chain comprising a sequence the sequence of SEQ ID NO: 3796, or (iv) a combination thereof.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3792; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3799 and a sequence having at least 50%, 55%, 60%, 65%, 70%, 75%, 80%, 85%, 90%, 95%, 96%, 97%, 98%, 99%, 99.5%. 99.7%, or 99.9% sequence identity to the sequence of SEQ ID NO: 3648; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to a sequence having at least 50%, 55%, 60%, 65%, 70%, 75%, 80%, 85%, 90%, 95%, 96%, 97% 98%, 99%, 99.5%, 99.7%, or 99.9% sequence identity to the sequence of SEQ ID NO: 3792; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3799 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3644, or (iv) a combination thereof. In some embodiments, the second polypeptide chain comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3799 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 via the sequence of SEQ ID NO: 3308.

In some embodiments, the multifunctional molecule comprises: (i) a first polypeptide chain comprising the sequence of SEQ ID NO: 1346 and the sequence of SEQ ID NO: 3792; (ii) a second polypeptide chain comprising the sequence of SEQ ID NO: 3799 and the sequence of SEQ ID NO: 3648; (iii) a third polypeptide chain comprising the sequence of SEQ ID NO: 1349 and the sequence of SEQ ID NO: 3644, or (iv) a combination thereof. In some embodiments, the second polypeptide chain further r comprises the sequence of SEQ ID NO: 3308.

In some embodiments, the multifunctional molecule comprises: (i) a first polypeptide chain comprising the sequence of SEQ ID NO: 1346 operatively linked to the sequence of SEQ ID NO: 3792; (ii) a second polypeptide chain comprising the sequence of SEQ ID NO: 3799 operatively linked to the sequence of SEQ ID NO: 3648; (iii) a third polypeptide chain comprising the sequence of SEQ ID NO: 1349 operatively linked to the sequence of SEQ ID NO: 3644, or (iv) a combination thereof. In some embodiments, the second polypeptide chain comprises the sequence of SEQ ID NO: 3799 operatively linked to the sequence of SEQ ID NO: 3648 via the sequence of SEQ ID NO: 3308.

In some embodiments, the multifunctional molecule comprises: (i) a first polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3791; (ii) a second polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3798; (iii) a third polypeptide chain comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3518, or (iv) a combination thereof.

In some embodiments, the multifunctional molecule comprises: (i) a first polypeptide chain comprising the sequence of SEQ ID NO: 3791; (ii) a second polypeptide chain comprising the sequence of SEQ ID NO: 3791; (iii) a third polypeptide chain comprising a sequence the sequence of SEQ ID NO: 3518, or (iv) a combination thereof.

In some embodiments, the TCRβV-binding moiety comprises: (i) a VH comprising a HC CDR1, a HC CDR2 and a HC CDR3 of SEQ ID NO: 1102, SEQ ID NO. 1103, and SEQ ID NO: 1104, respectively: a HC CDR1, a HC CDR2 and a HC CDR3 of SEQ ID NO: 1105, SEQ ID NO: 1106, and SEQ ID NO: 1104, respectively; or a HC CDR1, a HC CDR2 and a HC CDR3 of SEQ ID NO: 3640, SEQ ID NO: 1103, and SEQ ID NO: 1104, respectively; (ii) a VL comprising a LC CDR1, a LC CDR2, and a LC of SEQ ID NO: SEQ ID NO: 1107, SEQ ID NO: 1108, and SEQ ID NO: 1109, respectively: a LC CDR1, a LC CDR2, and a LC of SEQ ID NO:1110, SEQ ID NO: 1108, and SEQ ID NO: 1109, respectively; or a LC CDR1, a LC CDR2, and a LC of SEQ ID NO:1107, SEQ ID NO: 1108, and SEQ ID NO: 1109, respectively; or (iii) a combination thereof.

In some embodiments, the TCRβV-binding moiety comprises a VH comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1113: a VL comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3797; or a combination thereof.

In some embodiments, the TCRβV-binding moiety comprises a VH comprising the sequence of SEQ ID NO: 1113: a VL comprising the sequence of SEQ ID NO: 3797; or a combination thereof.

In some embodiments, an antibody or antigen binding fragment thereof comprises: (i) a VH comprising a HC CDR1, a HC CDR2 and a HC CDR3 of SEQ ID NO: 1102, SEQ ID NO: 1103, and SEQ ID NO: 1104, respectively: a HC CDR1, a HC CDR2 and a HC CDR3 of SEQ ID NO: 1105, SEQ ID NO: 1106, and SEQ ID NO: 1104, respectively; or a HC CDR1, a HC CDR2 and a HC CDR3 of SEQ ID NO: 3640, SEQ ID NO: 1103, and SEQ ID NO: 1104, respectively; (ii) a VL comprising a LC CDR1, a LC CDR2, and a LC of SEQ ID NO: SEQ ID NO: 1107, SEQ ID NO: 1108, and SEQ ID NO: 1109, respectively; a LC CDR1, a LC CDR2, and a LC of SEQ ID NO:1110, SEQ ID NO: 1108, and SEQ ID NO: 1109, respectively; or a LC CDR1, a LC CDR2, and a LC of SEQ ID NO: 1107, SEQ ID NO: 1108, and SEQ ID NO: 1109, respectively; or (iii) a combination thereof.

In some embodiments, an antibody or antigen binding fragment thereof comprises a VH comprising a sequence having at least 50%, 55%, 60%, 65%, 70%, 75%, 80%, 85%, 90%, 95%, 96%, 97%, 98%, 99%. 99.5%, 99.7%, or 99.9% sequence identity to the sequence of SEQ ID NO: 1113; a VL comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3797; or a combination thereof.

In some embodiments, an antibody or antigen binding fragment thereof comprises a VH comprising the sequence of SEQ ID NO: 1113: a VL comprising the sequence of SEQ ID NO: 3797; or a combination thereof.

In some embodiments, the tumor-associated antigen binding moiety comprises a VH comprising a HC CDR1, a HC CDR2 and a HC CDR3 comprising the sequences of SEQ ID NO: 524, SEQ ID NO: 525, and SEQ ID NO: 526 or sequences thereof with no more than 1, 2, 3, or 4 mutations, such as substitutions, additions, or deletions, respectively, or a VH comprising a HC CDR1, a HC CDR2 and a HC CDR3 comprising the sequences of SEQ ID NO: 538, SEQ ID NO: 539, and SEQ ID NO: 540 or sequences thereof with no more than 1, 2, 3, or 4 mutations, such as substitutions, additions, or deletions, respectively.

In some embodiments, the tumor-associated antigen binding moiety comprises a VL comprising a LC CDR1, a LC CDR2 and a LC CDR3 comprising the sequences of SEQ ID NO: 1168, SEQ ID NO: 1295, and SEQ ID NO: 528 or sequences thereof with no more than 1, 2, 3, or 4 mutations, such as substitutions, additions, or deletions, respectively, or a VL comprising a LC CDR1, a LC CDR2 and a LC CDR3 comprising the sequences of SEQ ID NO: 562, SEQ ID NO: 563, and SEQ ID NO: 564 or sequences thereof with no more than 1, 2, 3, or 4 mutations, such as substitutions, additions, or deletions, respectively.

In some embodiments, the tumor-associated antigen binding moiety comprises a VH comprising a HC CDR1, a HC CDR2 and a HC CDR3 comprising the sequences of SEQ ID NO: 524, SEQ ID NO: 525, and SEQ ID NO: 526 or sequences thereof with no more than 1, 2, 3, or 4 mutations, such as substitutions, additions, or deletions, respectively, and a VL comprising a LC CDR1, a LC CDR2 and a LC CDR3 comprising the sequences of SEQ ID NO: 1168, SEQ ID NO: 1295, and SEQ ID NO: 528 or sequences thereof with no more than 1, 2, 3, or 4 mutations, such as substitutions, additions, or deletions, respectively.

In some embodiments, the tumor-associated antigen binding moiety comprises a VH comprising a HC CDR1, a HC CDR2 and a HC CDR3 comprising the sequences of SEQ ID NO: 538, SEQ ID NO: 539, and SEQ ID NO: 540 or sequences thereof with no more than 1, 2, 3, or 4 mutations, such as substitutions, additions, or deletions, respectively, and a VL comprising a LC CDR1, a LC CDR2 and a LC CDR3 comprising the sequences of SEQ ID NO: 562, SEQ ID NO: 563, and SEQ ID NO: 564 or sequences thereof with no more than 1, 2, 3, or 4 mutations, such as substitutions, additions, or deletions, respectively.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 523 or the sequence of SEQ ID NO: 537.

In some embodiments, the tumor-associated antigen binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 527 or the sequence of SEQ ID NO: 561.

In some embodiments, the tumor-associated antigen binding moiety comprises a VH comprising the sequence of SEQ ID NO: 523 or the sequence of SEQ ID NO: 537.

In some embodiments, the tumor-associated antigen binding moiety comprises a VL comprising the sequence of SEQ ID NO: 527 or the sequence of SEQ ID NO: 561.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 523 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 527.

In some embodiments, the tumor-associated antigen binding moiety comprises a VH comprising the sequence of SEQ ID NO: 523 and a VL comprising the sequence of SEQ ID NO: 527.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 537 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 561.

In some embodiments, the tumor-associated antigen binding moiety comprises a VH comprising the sequence of SEQ ID NO: 537 and a VL comprising the sequence of SEQ ID NO: 561.

In some embodiments, the tumor-associated antigen binding moiety comprises a VH comprising a HC CDR1, a HC CDR2 and a HC CDR3 comprising the sequences of SEQ ID NO: 581, SEQ ID NO: 582, and SEQ ID NO: 583, respectively.

In some embodiments, the tumor-associated antigen binding moiety comprises a VL comprising a LC CDR1, a LC CDR2 and a LC CDR3 comprising the sequences of SEQ ID NO: 586, SEQ ID NO: 587, and SEQ ID NO: 588, respectively.

In some embodiments, the tumor-associated antigen binding moiety comprises a VH comprising a HC CDR1, a HC CDR2 and a HC CDR3 comprising the sequences of SEQ ID NO: 581, SEQ ID NO: 582, and SEQ ID NO: 583, respectively, and a VL comprising a LC CDR1, a LC CDR2 and a LC CDR3 comprising the sequences of SEQ ID NO: 586, SEQ ID NO: 587, and SEQ ID NO: 588, respectively.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 580.

In some embodiments, the tumor-associated antigen binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 585.

In some embodiments, the tumor-associated antigen binding moiety comprises a VH comprising the sequence of SEQ ID NO: 580.

In some embodiments, the tumor-associated antigen binding moiety comprises a VL comprising the sequence of SEQ ID NO: 585.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 580 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 585.

In some embodiments, the tumor-associated antigen binding moiety comprises a VH comprising the sequence of SEQ ID NO. 580 and a VL comprising the sequence of SEQ ID NO: 585.

In some embodiments, the TCRβV-binding moiety comprises a VH comprising a HC CDR1, a HC CDR2 and a HC CDR3 comprising the sequences of SEQ ID NO: 3650, SEQ ID NO: 3651, and SEQ ID NO: 5, respectively, or a VH comprising a HC CDR1, a HC CDR2 and a HC CDR3 comprising the sequences of SEQ ID NO: 542, SEQ ID NO: 543, and SEQ ID NO: 544, respectively.

In some embodiments, the TCRβV-binding moiety comprises a VL comprising a LC CDR1, a LC CDR2 and a LC CDR3 comprising the sequences of SEQ ID NO: 3655, SEQ ID NO: 3653, and SEQ ID NO: 8, respectively, or a VL comprising a LC CDR1, a LC CDR2 and a LC CDR3 comprising the sequences of SEQ ID NO: 545, SEQ ID NO: 546, and SEQ ID NO: 547, respectively.

In some embodiments, the TCRβV-binding moiety comprises a VH comprising a HC CDR1, a HC CDR2 and a HC CDR3 comprising the sequences of SEQ ID NO: 3650, SEQ ID NO: 3651, and SEQ ID NO: 5, respectively, and a VL comprising a LC CDR1, a LC CDR2 and a LC CDR3 comprising the sequences of SEQ ID NO: 3655, SEQ ID NO: 3653, and SEQ ID NO: 8, respectively.

In some embodiments, the TCRβV-binding moiety comprises a VH comprising a HC CDR1, a HC CDR2 and a HC CDR3 comprising the sequences of SEQ ID NO: 542, SEQ ID NO: 543, and SEQ ID NO: 544, respectively, and a VL comprising a LC CDR1, a LC CDR2 and a LC CDR3 comprising the sequences of SEQ ID NO: 545, SEQ ID NO: 546, and SEQ ID NO: 547, respectively.

In some embodiments, the TCRβV-binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 1346 or the sequence of SEQ ID NO: 541.

In some embodiments, the TCRβV-binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 1349 or the sequence of SEQ ID NO: 3527.

In some embodiments, the TCRβV-binding moiety comprises a VH comprising the sequence of SEQ ID NO: 1346 or the sequence of SEQ ID NO: 541.

In some embodiments, the TCRβV-binding moiety comprises a VL comprising the sequence of SEQ ID NO: 1349 or the sequence of SEQ ID NO: 3527.

In some embodiments, the TCRβV-binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 1346 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 1349.

In some embodiments, the TCRβV-binding moiety comprises a VH comprising the sequence of SEQ ID NO: 1346 and a VL comprising the sequence of SEQ ID NO: 1349.

In some embodiments, the TCRβV-binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 541 and a VL comprising a sequence having at least 70% sequence identity the sequence of SEQ ID NO: 3527.

In some embodiments, the TCRβV-binding moiety comprises a VH comprising the sequence of SEQ ID NO: 541 and a VL comprising the sequence of SEQ ID NO: 3527.

In some embodiments, the TCRβV-binding moiety comprises an scFv comprising the sequence of SEQ ID NO: 1346 operatively linked to the sequence of SEQ ID NO: 1349 via a linker comprising the sequence of SEQ ID NO: 3801.

In some embodiments, the TCRβV-binding moiety comprises an scFv comprising the sequence of SEQ ID NO: 541 operatively linked to the sequence of SEQ ID NO: 3527 via a linker comprising the sequence of SEQ ID NO: 3801.

In some embodiments, the TCRβV-binding moiety comprises an scFv comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 1331 or the sequence of SEQ ID NO: 1376.

In some embodiments, the TCRβV-binding moiety comprises an scFv comprising the sequence of SEQ ID NO: 1331 or the sequence of SEQ ID NO: 1376.

In some embodiments, the multifunctional molecule consists of a first polypeptide chain, a second polypeptide chain, and a third polypeptide chain, wherein:
(i) the first polypeptide chain comprises the sequence of SEQ ID NO: 523, and the sequence of SEQ ID NO: 3649;
(ii) the second polypeptide chain comprises the sequence of SEQ ID NO: 1331, the sequence of SEQ ID NO: 2270, and the sequence of SEQ ID NO: 3648, and
(iii) the third polypeptide chain comprises the sequence of SEQ ID NO: 527, and the sequence of SEQ ID NO: 3644.

In some embodiments, the multifunctional molecule consists of a first polypeptide chain, a second polypeptide chain, and a third polypeptide chain, wherein:
(i) the first polypeptide chain comprises the sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO. 2270 operatively linked to the sequence of SEQ ID NO: 3648; and
(iii) the third polypeptide chain comprises the sequence of SEQ ID NO: 527 operatively linked to the sequence of SEQ ID NO: 3644.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3800; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 501;
(ii) the second polypeptide chain comprises the sequence of SEQ ID NO: 3800; and
(iii) the third polypeptide chain comprises the sequence of SEQ ID NO: 502.

In some embodiments, the multifunctional molecule consists of a first polypeptide chain, a second polypeptide chain, and a third polypeptide chain, wherein:
(i) the first polypeptide chain comprises a first sequence of SEQ ID NO: 523, a second sequence of SEQ ID NO: 523, and the sequence of SEQ ID NO: 3649;
(ii) the second polypeptide chain comprises the sequence of SEQ ID NO: 1331, the sequence of SEQ ID NO: 2270, and the sequence of SEQ ID NO: 3648; and
(iii) the third polypeptide chain comprises the sequence of SEQ ID NO: 527, and the sequence of SEQ ID NO: 3644.

In some embodiments, the multifunctional molecule consists of a first polypeptide chain, a second polypeptide chain, and a third polypeptide chain, wherein:
(i) the first polypeptide chain comprises the first sequence of SEQ ID NO: 523 operatively linked to the second sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO: 2270 operatively linked to the sequence of SEQ ID NO: 3648; and
(iii) the third polypeptide chain comprises the sequence of SEQ ID NO: 527 operatively linked to the sequence of SEQ ID NO. 3644.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 590;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3800; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 590;
(ii) the second polypeptide chain comprises the sequence of SEQ ID NO: 3800; and
(iii) the third polypeptide chain comprises the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 537, and the sequence of SEQ ID NO: 559;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 561, and the sequence of SEQ ID NO: 558.

In some embodiments, the multifunctional molecule consists of a first polypeptide chain, a second polypeptide chain, and a third polypeptide chain, wherein:
(i) the first polypeptide chain comprises the sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 509;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 511; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 509;
(ii) the second polypeptide chain comprises the sequence of SEQ ID NO: 511; and
(iii) the third polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises a first sequence of SEQ ID NO: 537, a second sequence of SEQ ID NO: 537, and the sequence of SEQ ID NO: 559;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 561, and the sequence of SEQ ID NO: 558.

In some embodiments, the multifunctional molecule consists of a first polypeptide chain, a second polypeptide chain, and a third polypeptide chain, wherein:
(i) the first polypeptide chain comprises the first sequence of SEQ ID NO: 537 operatively linked to the second sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 512;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 511; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 512;
(ii) the second polypeptide chain comprises the sequence of SEQ ID NO: 511; and
(iii) the third polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 580, and the sequence of SEQ ID NO: 584;
(ii) the second polypeptide chain comprises the sequence of SEQ ID NO: 1376, the sequence of SEQ ID NO: 2191, and the sequence of SEQ ID NO: 3533; and
(iii) the third polypeptide chain comprises the sequence of SEQ ID NO: 585, and the sequence of SEQ ID NO: 3528.

In some embodiments, the multifunctional molecule consists of a first polypeptide chain, a second polypeptide chain, and a third polypeptide chain, wherein:
(i) the first polypeptide chain comprises the sequence of SEQ ID NO: 580 operatively linked to the sequence of SEQ ID NO: 584;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 585 operatively linked to the sequence of SEQ ID NO: 3528.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 519;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 511; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 520.

In some embodiments, the multifunctional molecule consists of a first polypeptide chain, a second polypeptide chain, and a third polypeptide chain, wherein:
(i) the first polypeptide chain comprises the sequence of SEQ ID NO: 519;
(ii) the second polypeptide chain comprises the sequence of SEQ ID NO: 511; and
(iii) the third polypeptide chain comprises the sequence of SEQ ID NO: 520.

In some embodiment, the multifunctional molecule as provided herein is Tristar molecule comprising a tumor-associated antigen binding moiety, at least one cytokine molecule or a functional fragment or functional variant thereof, and a TCRβV-binding moiety covalently linked to the at least one cytokine molecule or a functional fragment or functional variant thereof.

In some embodiments, the tumor-associated antigen binding moiety comprises an antibody or antigen binding fragment thereof, wherein the antigen binding fragment comprises any one selected from the group consisting of a Fab, a F(ab′)2, an Fv, a single chain Fv (scFv), a single domain antibody, a diabody (dAb), a camelid antibody, and a combination thereof.

In some embodiments, the tumor-associated antigen binding moiety comprises a VH comprising a HC CDR1, a HC CDR2 and a HC CDR3 comprising any one of the HC CDR1 sequences, any one of the HC CDR2 sequences, and any one of the HC CDR3 sequences listed in Table 22 or sequences thereof with no more than 1, 2, 3, or 4 mutations, such as substitutions, additions, or deletions. In some embodiments, the tumor-associated antigen binding moiety comprises a VL comprising a LC CDR1, a LC CDR2 and a LC CDR3 comprising any one of the LC CDR1 sequences, any one of the LC CDR2 sequences, and any one of the LC CDR3 sequences listed in Table 22 or sequences thereof with no more than 1, 2, 3, or 4 mutations, such as substitutions, additions, or deletions.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VH sequences listed in Table 22. In some embodiments, the tumor-associated antigen binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VL sequences listed in Table 22. In some embodiments, the tumor-associated antigen binding moiety comprises a VH comprising any one of the VH sequences listed in Table 22. In some embodiments, the tumor-associated antigen binding moiety comprises a VL comprising any one of the VL sequences listed in Table 22.

In some embodiments, the TCRβV-binding moiety comprises an antibody or antigen binding fragment thereof, wherein the antigen binding fragment comprises any one selected from the group consisting of a Fab, a F(ab′)2, an Fv, a single chain Fv (scFv), a single domain antibody, a diabody (dAb), a camelid antibody, and a combination thereof.

In some embodiments, the TCRβV-binding moiety comprises a VH comprising a HC CDR1, a HC CDR2 and a HC CDR3 comprising any one of the HC CDR1 sequences, any one of the HC CDR2 sequences, and any one of the HC CDR3 sequences listed in Table 22 or sequences thereof with no more than 1, 2, 3, or 4 mutations, such as substitutions, additions, or deletions. In some embodiments, the TCRβV-binding moiety comprises a VL comprising a LC CDR1, a LC CDR2 and a LC CDR3 comprising any one of the LC CDR1 sequences, any one of the LC CDR2 sequences, and any one of the LC CDR3 sequences listed in Table 22 or sequences thereof with no more than 1, 2, 3, or 4 mutations, such as substitutions, additions, or deletions.

In some embodiments, the TCRβV-binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VH sequences listed in Table 22. In some embodiments, the TCRβV-binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VL sequences listed in Table 22. In some embodiments, the TCRβV-binding moiety comprises a VH comprising any one of the VH sequences listed in Table 22. In some embodiments, the TCRβV-binding moiety comprises a VL comprising any one of the VL sequences listed in Table 22.

In some embodiments, the at least one cytokine molecule or a functional fragment or functional variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the cytokine sequences listed in Table 22. In some embodiments, the at least one cytokine molecule or a functional fragment or functional variant thereof comprises any one of the cytokine sequences listed in Table 22.

In some embodiments, the multifunctional molecule comprises one or more components comprising sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the components listed in Table 22, or any combination thereof.

In some embodiments, the multifunctional molecule comprises one or more polypeptide chains comprising sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the polypeptide chain sequences listed in Table 22, or any combination thereof.

In some embodiments, the multifunctional molecule comprises one or more components listed in Table 22, or any combination thereof. In some embodiments, the multifunctional molecule comprises one or more polypeptide chains listed in Table 22, or any combination thereof.

In another aspect, provided herein is a multifunctional molecule that is a T-cell engager (TCE) comprising a tumor-associated antigen binding moiety and a TCRβV-binding moiety. In some embodiments, the tumor-associated antigen binding moiety comprises a VH comprising a HC CDR1, a HC CDR2 and a HC CDR3 comprising any one of the HC CDR1 sequences, any one of the HC CDR2 sequences, and any one of the HC CDR3 sequences listed in Table 22 or sequences thereof with no more than 1, 2, 3, or 4 mutations, such as substitutions, additions, or deletions.

In some embodiments, the tumor-associated antigen binding moiety comprises a VL comprising a LC CDR1, a LC CDR2 and a LC CDR3 comprising any one of the LC CDR1 sequences, any one of the LC CDR2 sequences, and any one of the LC CDR3 sequences listed in Table 22 or sequences thereof with no more than 1, 2, 3, or 4 mutations, such as substitutions, additions, or deletions.

In some embodiments, the tumor-associated antigen binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VH sequences listed in Table 22. In some embodiments, the tumor-associated antigen binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VL sequences listed in Table 22. In some embodiments, the tumor-associated antigen binding moiety comprises a VH comprising any one of the VH sequences listed in Table 22. In some embodiments, the tumor-associated antigen binding moiety comprises a VL comprising any one of the VL sequences listed in Table 22.

In some embodiments, the TCRβV-binding moiety comprises an antibody or antigen binding fragment thereof, wherein the antigen binding fragment comprises any one selected from the group consisting of a Fab, a F(ab′)2, an Fv, a single chain Fv (scFv), a single domain antibody, a diabody (dAb), a camelid antibody, and a combination thereof.

In some embodiments, the TCRβV-binding moiety comprises a VH comprising a HC CDR1, a HC CDR2 and a HC CDR3 comprising any one of the HC CDR1 sequences, any one of the HC CDR2 sequences, and any one of the HC CDR3 sequences listed in Table 22 or sequences thereof with no more than 1, 2, 3, or 4 mutations, such as substitutions, additions, or deletions. In some embodiments, the TCRβV-binding moiety comprises a VL comprising a LC CDR1, a LC CDR2 and a LC CDR3 comprising any one of the LC CDR1 sequences, any one of the LC CDR2 sequences, and any one of the LC CDR3 sequences listed in Table 22 or sequences thereof with no more than 1, 2, 3, or 4 mutations, such as substitutions, additions, or deletions.

In some embodiments, the TCRβV-binding moiety comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VH sequences listed in Table 22. In some embodiments, the TCRβV-binding moiety comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the VL sequences listed in Table 22. In some embodiments, the TCRβV-binding moiety comprises a VH comprising any one of the VH sequences listed in Table 22. In some embodiments, the TCRβV-binding moiety comprises a VL comprising any one of the VL sequences listed in Table 22.

In some embodiments, the multifunctional molecule comprises one or more components comprising sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the components listed in Table 22, or any combination thereof.

In some embodiments, the multifunctional molecule comprises one or more polypeptide chains comprising sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of the polypeptide chain sequences listed in Table 22, or any combination thereof.

In some embodiments, the multifunctional molecule comprises one or more components listed in Table 22, or any combination thereof. In some embodiments, the multifunctional molecule comprises one or more polypeptide chains listed in Table 22, or any combination thereof.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 503; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502.

In some embodiments, the multifunctional molecule consists of a first polypeptide chain, a second polypeptide chain, and a third polypeptide chain, wherein:
(i) the first polypeptide chain comprises the sequence of SEQ ID NO: 501;
(ii) the second polypeptide chain comprises the sequence of SEQ ID NO: 503; and
(iii) the third polypeptide chain comprises the sequence of SEQ ID NO: 502.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 504;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 504;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 504;
(ii) the second polypeptide chain comprises the sequence of SEQ ID NO: 504;
(iii) the third polypeptide chain comprises the sequence of SEQ ID NO: 502; and
(iv) the fourth polypeptide chain comprises the sequence of SEQ ID NO: 502.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 508;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 506;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 507.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 508;
(ii) the second polypeptide chain comprises the sequence of SEQ ID NO: 506;
(iii) the third polypeptide chain comprises the sequence of SEQ ID NO: 502; and
(iv) the fourth polypeptide chain comprises the sequence of SEQ ID NO: 507.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 509;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 513; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, and a third polypeptide chain, wherein:
(i) the first polypeptide chain comprises the sequence of SEQ ID NO: 509.
(ii) the second polypeptide chain comprises the sequence of SEQ ID NO: 513; and
(iii) the third polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 512;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 513.

In some embodiments, the multifunctional molecule consists of a first polypeptide chain, a second polypeptide chain, and a third polypeptide chain, wherein:
(i) the first polypeptide chain comprises the sequence of SEQ ID NO: 512;
(ii) the second polypeptide chain comprises the sequence of SEQ ID NO: 513; and
(iii) the third polypeptide chain comprises the sequence of SEQ ID NO: 510.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 514;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 514;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, a third polypeptide chain, and a fourth polypeptide chain wherein.
(i) the first polypeptide chain comprises the sequence of SEQ ID NO: 514;
(ii) the second polypeptide chain comprises the sequence of SEQ ID NO: 514;
(iii) the third polypeptide chain comprises the sequence of SEQ ID NO: 510; and
(iv) the fourth polypeptide chain comprises the sequence of SEQ ID NO: 510.

In some embodiments, the multifunctional molecule consists of a first polypeptide chain, a second polypeptide chain, and a third polypeptide chain, wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 519;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 513; and
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 520.

In some embodiments, the multifunctional molecule consists of a first polypeptide chain, a second polypeptide chain, and a third polypeptide chain, wherein:
(i) the first polypeptide chain comprises the sequence of SEQ ID NO: 519;
(ii) the second polypeptide chain comprises the sequence of SEQ ID NO: 520; and
(iii) the third polypeptide chain comprises the sequence of SEQ ID NO: 513.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 521;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 521;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 520; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 520.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 521;
(ii) the second polypeptide chain comprises the sequence of SEQ ID NO: 521;
(iii) the third polypeptide chain comprises the sequence of SEQ ID NO: 520; and
(iv) the fourth polypeptide chain comprises the sequence of SEQ ID NO: 520.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 505;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 506;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 502; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 507.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 505;
(ii) the second polypeptide chain comprises the sequence of SEQ ID NO: 506;
(iii) the third polypeptide chain comprises the sequence of SEQ ID NO: 502; and
(iv) the fourth polypeptide chain comprises the sequence of SEQ ID NO: 507.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 515;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 517;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 516; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 515;
(ii) the second polypeptide chain comprises the sequence of SEQ ID NO: 517;
(iii) the third polypeptide chain comprises the sequence of SEQ ID NO: 516; and
(iv) the fourth polypeptide chain comprises the sequence of SEQ ID NO: 510.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 515;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 518;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 516; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 510.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 515;
(ii) the second polypeptide chain comprises the sequence of SEQ ID NO: 518;
(iii) the third polypeptide chain comprises the sequence of SEQ ID NO: 516; and
(iv) the fourth polypeptide chain comprises the sequence of SEQ ID NO: 510.

In some embodiments, the multifunctional molecule consists of a first polypeptide chain, a second polypeptide chain, a third polypeptide chain, and a fourth polypeptide chain wherein:
(i) the first polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 515;
(ii) the second polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 522;
(iii) the third polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 516; and
(iv) the fourth polypeptide chain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 512.

In some embodiments, the multifunctional molecule consists of a first polypeptide chain, a second polypeptide chain, a third polypeptide chain, and a fourth polypeptide chain wherein:
(i) the first polypeptide chain comprises the sequence of SEQ ID NO: 515.
(ii) the second polypeptide chain comprises the sequence of SEQ ID NO: 522;
(iii) the third polypeptide chain comprises the sequence of SEQ ID NO: 516; and
(iv) the fourth polypeptide chain comprises the sequence of SEQ ID NO: 520.

In some embodiments, the multispecific (e.g., bi-, tri-, quad-specific) or multifunctional molecule comprises a T cell engager that binds to, e.g., activates a costimulatory molecule expressed by a T cell. In some embodiments, the T cell engager that binds to, e.g., activates, a costimulatory molecule expressed by a T cell is a cytokine, such as IL-2, IL-15, IL-15-sushi, or IL21. In some embodiments, the T cell engager that binds to, e.g., activates, a costimulatory molecule expressed by a T cell binds to, e.g., activates, CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager that binds to, e.g., activates, a costimulatory molecule expressed by a T cell is a CD40 ligand (CD40L), a CD70 ligand; an scFv or antigen binding domain of an antibody molecule that binds to CD40, an scFv or antigen binding domain of an antibody molecule that binds to CD70; an scFv or antigen binding domain of an antibody molecule that binds to OX40; an OX40 ligand (OX40L); a 41BB ligand (41BBL); or an scFv or antigen binding domain of an antibody molecule that binds to a CD2.

In some embodiments, the multispecific (e.g., bi-, tri-, quad-specific) or multifunctional molecule comprises at least two T cell engagers, wherein one of the T cell engagers binds to, e.g., activates a costimulatory molecule expressed by a T cell, and one of the T cell engagers binds to, e.g., activates TCRB, e.g., a TCRβV region. For example, the multispecific (e.g., bi-, tri-, quad-specific) or multifunctional molecule can comprise at least two T cell engagers, wherein one of the T cell engagers is an scFv or antigen binding domain of an antibody molecule that binds to, e.g., activates TCRB, e.g., a TCRβV region; and wherein one of the T cell engagers is a cytokine, such as IL-2, IL-15, IL-15-sushi, or IL21, a CD40 ligand (CD40L), a CD70 ligand; an scFv or antigen binding domain of an antibody molecule that binds to CD40, an scFv or antigen binding domain of an antibody molecule that binds to CD70; an scFv or antigen binding domain of an antibody molecule that binds to OX40; an OX40 ligand (OX40L): a 41BB ligand (41BBL); or an scFv or antigen binding domain of an antibody molecule that binds to a CD2, that binds to, e.g., activates a costimulatory molecule expressed by a T cell.

In some embodiments, the TCRβV-binding moiety comprises a KD to a TCRβV that is less than 0.001 pM, 0.005 pM, 0.01 pM, 0.05 pM, 0.1 pM, 0.5 pM, 1 pM, 5 pM, 10 pM, 50 pM, 100 pM, 500 pM, 1 nM, 5 nM, 10 nM, 50 nM, 100 nM, 500 nM, 1 pM, 5 μM, or is less than 10 μM.

In some embodiments, the TCRβV-binding moiety comprises a KD to a TCRβV that is more than 0.001 pM, 0.005 pM, 0.01 pM, 0.05 pM, 0.1 pM, 0.5 pM, 1 pM, 5 pM, 10 pM, 50 pM, 100 pM, 500 pM, 1 nM, 5 nM, 10 nM, 50 nM, 100 nM, 500 nM, 1 μM, 5 μM, or more than 10 μM.

In some embodiments, the TCRβV-binding moiety comprises a KD to a TCRβV that is from 0.001 pM-10 μM, 0.005 pM-10 pM, 0.01 pM-10 pM, 0.05 pM-10 pM, 0.1 pM-10 μM. 0.5 pM-10 pM, 1 pM-10 AM, 5 pM-10 pM, 10 pM-10 μM. 50 pM-10 pM, 100 pM-10 pM, 500 pM-10 pM, 1 nM-10 μM. 5 nM-10 AM, 10 nM-10 AM, 50 nM-10 μM. 100 nM-10 μM, 500 nM-10 μM, IuM-10 μM, or from 5 pM-10 u.M.

In some embodiments, the TCRβV-binding moiety comprises a KD to a TCRβV that is from 0.001 pM-10 μM, 0.001 pM-5 μM. 0.001 pM-luM. 0.001 pM-500 nM, 0.001 pM-100 nM, 0.001 pM-50 nM, 0.001 pM-10 nM, 0.001 pM-5 nM, 0.001 pM-1 nM, 0.001 pM-500 pM, 0.001 pM-100 pM, 0.001 pM-50 pM, 0.001 pM-10 pM, 0.001 pM-5 pM, 0.001 pM-1 pM, 0.001 pM-0.5 pM, 0.001 pM-0.1 pM, 0.001 pM-0.05 pM, or from 0.001 pM-0.01 pM.

In some embodiments, the TCRβV-binding moiety comprises a KD to a TCRβV that is from 0.001 pM-0.005 pM, 0.005 pM-0.01 pM, 0.01 pM-0.05 pM, 0.05 pM-0.1 pM, 0.1 pM-0.5 pM, 0.5 pM-1 pM, 1 pM-5 pM, 5 pM-10 pM, 10 pM-50 pM, 50 pM-100 pM, 100 pM-500 pM, 500 pM-1 nM, 1 nM-5 nM, 5 nM-10 nM, 10 nM-50 nM, 50 nM-100 nM, 100 nM-500 nM, 500 nM-1 μM, IuM-SuM, or from 5 μM-10 μM.

In some embodiments, the TCRβV-binding moiety comprises a KD to a TCRβV that is from 0.001 pM-10 μM, 0.005 pM-5 μM, 0.01 pM-1000 M, 0.05 pM-500 nM, 0.1 pM-100 nM, 0.5 pM-50 nM, 1 pM-10 nM, 5 pM-5 nM, 10 pM-1 nM, or from 50 pM-500 pM.

In some embodiments, the TCRβV-binding moiety comprises a to a TCRβV that from 10 pM-10 μM, 50 pM-5 pM, 100 pM-1000 nM, 500 pM-100 nM, or from 1 nM-10 nM.

In another aspect, provided herein is the use of the multifunctional molecule as provided herein, the polynucleotide as provided herein, the vector as provided herein, the cell as provided herein, the pharmaceutical composition as provided herein, or any combination thereof for a method of treating a condition or disease in a subject in need thereof, wherein the method comprises administering to the subject a therapeutically effective amount of the multifunctional molecule as provided herein, the polynucleotide as provided herein, the vector as provided herein, the cell as provided herein, the pharmaceutical composition as provided herein, or any combination thereof, thereby treating the condition or disease in the subject.

In another aspect, provided herein is the use of the multifunctional molecule as provided herein, the polynucleotide as provided herein, the vector as provided herein, the cell as provided herein, the pharmaceutical composition as provided herein, or any combination thereof for treating a condition or disease in a subject in need thereof, wherein the use of the multifunctional molecule as provided herein, the polynucleotide as provided herein, the vector as provided herein, the cell as provided herein, the pharmaceutical composition as provided herein, or any combination thereof, or any combination thereof is formulated for administration to the subject in a therapeutically effective amount.

In some embodiments, the therapeutically effective amount is an amount sufficient to treat the condition or disease in the subject.

In another aspect, provided herein is the multifunctional molecule as provided herein, the polynucleotide as provided herein, the vector as provided herein, the cell as provided herein, the pharmaceutical composition as provided herein, or any combination thereof for use as a medicament.

In another aspect, provided herein is the multifunctional molecule as provided herein, the polynucleotide as provided herein, the vector as provided herein, the cell as provided herein, the pharmaceutical composition as provided herein, or any combination thereof for use in the manufacture of a medicament for treating a condition or disease in a subject in need thereof.

In some embodiments, the multifunctional molecule as provided herein, the polynucleotide as provided herein, the vector as provided herein, the cell as provided herein, the pharmaceutical composition as provided herein, or any combination thereof is formulated for administration to the subject in a therapeutically effective amount.

In some embodiments, the therapeutically effective amount is an amount sufficient to treat the condition or disease in the subject.

In another aspect, provided herein a composition comprising a first agent comprising a TCRβV-binding moiety as provided herein and at least one cytokine molecule or a functional fragment or functional variant thereof, and a second agent comprising a TCRβV-binding moiety as provided herein and a tumor-associated antigen binding moiety as provided herein. In some embodiments, the TCRβV-binding moiety in the first agent is same as the TCRβV-binding moiety in the second agent. In some embodiments, the TCRβV-binding moiety in the first agent is different from the TCRβV-binding moiety in the second agent. In some embodiments, the first agent and/or the second agent is a polypeptide molecule. In some embodiments, the first agent and/or the second agent is a multifunctional molecule. In some embodiments, the first agent and/or the second agent is a multispecific molecule.

In another aspect, provided herein a method of administering a first agent comprising a TCRβV-binding moiety as provided herein and at least one cytokine molecule or a functional fragment or functional variant thereof, and a second agent comprising a TCRβV-binding moiety as provided herein and a tumor-associated antigen binding moiety as provided herein to treat a disease or condition in a subject in need thereof. In some embodiments, the TCRβV-binding moiety in the first agent is same as the TCRβV-binding moiety in the second agent. In some embodiments, the TCRβV-binding moiety in the first agent is different from the TCRβV-binding moiety in the second agent. In some embodiments, the first agent is administered to the subject prior to the second agent. In some embodiments, the first agent is administered concurrently with the second agent to the subject. In some embodiments, the first agent is administered to the subject after the second agent (for example, see, FIG. 138 and FIG. 139).

What is claimed is:

1.-142. (canceled)

143. A multispecific molecule comprising:
(a) a tumor-associated antigen binding moiety;
(b) at least one cytokine molecule or a functional fragment or variant thereof; and
(c) a TCRβV-binding moiety, wherein the TCRβV-binding moiety is covalently linked to the at least one cytokine molecule or a functional fragment or variant thereof.

144. The multispecific molecule of claim 143, wherein the multispecific molecule comprises a first polypeptide chain comprising c, and a second polypeptide chain comprising an Fc domain;
wherein the first polypeptide chain and the second polypeptide chain are non-contiguous; and
wherein the tumor-associated antigen binding moiety is linked to the Fc domain of the first polypeptide chain, and the at least one cytokine molecule or a functional fragment or variant thereof is linked to the Fc domain of the second polypeptide chain.

145. The multispecific molecule of claim 144, wherein the Fc domain of the first polypeptide chain and the Fc domain of the second polypeptide chain are dimerized.

146. The multispecific molecule of claim 143, wherein the tumor-associated antigen binding moiety and the TCRβV-binding moiety comprises an antibody or antigen binding fragment thereof,
wherein the antigen binding fragment comprises any one selected from the group consisting of a Fab, a F(ab′)2, an Fv, a single chain Fv (scFv), a single domain antibody, a diabody (dAb), a camelid antibody, and any combination thereof.

147. The multispecific molecule of claim 143, wherein the tumor-associated antigen binding moiety comprises a Fab and the TCRβV-binding moiety comprises a scFv.

148. The multispecific molecule of claim 143, wherein the tumor-associated antigen binding moiety binds to a cancer antigen selected from the group consisting of CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100/pmel17), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1/LAGE-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, Survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RUI), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), FcRH5, PDL1, CD47, prostate specific membrane antigen (PMSA), prostate-specific antigen (PSA), Ron Kinase, c-Met, Immature laminin receptor, TAG-72, BING-4, Calcium-activated chloride channel 2, Cyclin-B1, 9D7, Ep-CAM, EphA3, SAP-1, PRAME, SSX-2, Melan-A/MART-1, TRP1/gp75, MC1R, β-catenin, BRCA1/2, CDK4, CML66, Fibronectin, Ras, TGF-B receptor, AFP, ETA, MAGE, CA-125, BAGE, GAGE, CDC27, a actinin-4, gangliosides, MART-2, MUC2, MUM1, MUM2, MUM3, NA88-1, NPM, OA1, OGT, RCC, RU11, RU12, SAGE, TRG, TSTA, L1-CAM, gpA33, GM2, VEGFR, Integrins, carbohydrates, TRAILR1, TRAILR2, RANKL, TGF-beta, hyaluronic acid, collagen, tenascin C, tenascin W, and immunoglobulin lambda-like polypeptide 1 (IGLL1).

149. The multispecific molecule of claim 143, wherein the tumor-associated antigen binding moiety binds to MSLN, CD20, or HER2.

150. The multispecific molecule of claim 143, wherein the tumor-associated antigen binding moiety comprises:
(i) a VH comprising a HC CDR1 comprising the sequence of SEQ ID NO: 524, a HC CDR2 comprising the sequence of SEQ ID NO: 525, and a HC CDR3 comprising the sequence of SEQ ID NO: 526, and a VL comprising a LC CDR1 comprising the sequence of SEQ ID NO: 1168, a LC CDR2 comprising the sequence of SEQ ID NO: 1295, and a LC CDR3 comprising the sequence of SEQ ID NO: 528;
(ii) a VH comprising a HC CDR1 comprising the sequence of SEQ ID NO: 538, a HC CDR2 comprising the sequence of SEQ ID NO: 539, and a HC CDR3 comprising the sequence of SEQ ID NO: 540, and a VL comprising a LC CDR1 comprising the sequence of SEQ ID NO: 562, a LC CDR2 comprising the sequence of SEQ ID NO: 563, and a LC CDR3 comprising the sequence of SEQ ID NO: 564; or
(iii) a VH comprising a HC CDR1 comprising the sequence of SEQ ID NO: 581, a HC CDR2 comprising the sequence of SEQ ID NO: 582, and a HC CDR3 comprising the sequence of SEQ ID NO: 583, and a VL comprising a LC CDR1 comprising the sequence of SEQ ID NO: 586, a LC CDR2 comprising the sequence of SEQ ID NO: 587, and a LC CDR3 comprising the sequence of SEQ ID NO: 588.

151. The multispecific molecule of claim 143, wherein the tumor-associated antigen binding moiety comprises:
(i) a VH comprising a sequence having at least 70% sequence identity to the sequence of SEQ ID NO: 523 and a VL comprising a sequence having at least 70% sequence identity to the sequence of SEQ ID NO: 527,
wherein the VH comprises a HC CDR1 comprising the sequence of SEQ ID NO: 524, a HC CDR2 comprising the sequence of SEQ ID NO: 525, and a HC CDR3 comprising the sequence of SEQ ID NO: 526, and the VL comprises a LC CDR1 comprising the sequence of SEQ ID NO: 1168, a LC CDR2 comprising the sequence of SEQ ID NO: 1295, and a LC CDR3 comprising the sequence of SEQ ID NO: 528;
(ii) a VH comprising a sequence having at least 70% sequence identity to the sequence of SEQ ID NO: 537 and a VL comprising a sequence having at least 70% sequence identity to the sequence of SEQ ID NO: 561,
wherein the VH comprises a HC CDR1 comprising the sequence of SEQ ID NO: 538, a HC CDR2 comprising the sequence of SEQ ID NO: 539, and a HC CDR3 comprising the sequence of SEQ ID NO: 540, and the VL comprises a LC CDR1 comprising the sequence of SEQ ID NO: 562, a LC CDR2 comprising the sequence of SEQ ID NO: 563, and a LC CDR3 comprising the sequence of SEQ ID NO: 564; or
(iii) a VH comprising a sequence having at least 70% sequence identity to the sequence of SEQ ID NO: 580 and a VL comprising a sequence having at least 70% sequence identity to the sequence of SEQ ID NO: 585,
wherein the VH comprises a HC CDR1 comprising the sequence of SEQ ID NO: 581, a HC CDR2 comprising the sequence of SEQ ID NO: 582, and a HC CDR3 comprising the sequence of SEQ ID NO: 583, and the VL comprises a LC CDR1 comprising the sequence of SEQ ID NO: 586, a LC CDR2 comprising the sequence of SEQ ID NO: 587, and a LC CDR3 comprising the sequence of SEQ ID NO: 588.

152. The multispecific molecule of claim 143, wherein the tumor-associated antigen binding moiety comprises:
(i) a VH comprising the sequence of SEQ ID NO: 523 and a VL comprising the sequence of SEQ ID NO: 527;
(ii) a VH comprising the sequence of SEQ ID NO: 537 and a VL comprising the sequence of SEQ ID NO: 561; or
(iii) a VH comprising the sequence of SEQ ID NO: 580 and a VL comprising the sequence of SEQ ID NO: 585.

153. The multispecific molecule of claim 143, wherein the TCRβV-binding moiety binds to one or more of a TCRβV subfamily selected from the group consisting of TCRβ V2 subfamily, TCRβ V3 subfamily, TCRβ V4 subfamily, TCRβ V5 subfamily, TCRβ V6 subfamily, TCRB V9 subfamily, TCRβ V10 subfamily, TCRβ V11 subfamily, TCRβ V12 subfamily, TCRβ V13 subfamily, TCRβ V16 subfamily, TCRβ V19, TCRβ V20 subfamily, TCRβ V21 subfamily, TCRβ V23 subfamily, TCRβ V27 subfamily, and TCRβ V28 subfamily.

154. The multispecific molecule of claim 143, wherein the TCRβV-binding moiety binds to TCRβV5 subfamily, TCRβV6 subfamily, TCRβV10 subfamily, TCRβV12 subfamily, or TCRβV20 subfamily.

155. The multispecific molecule of claim 143, wherein the TCRβV-binding moiety comprises:
(i) a VH comprising a HC CDR 1 comprising the sequence of SEQ ID NO: 3650, a HC CDR2 comprising the sequence of SEQ ID NO: 3651, and a HC CDR3 comprising the sequence of SEQ ID NO: 5, and a VL comprising a LC CDR1 comprising the sequence of SEQ ID NO: 3655, a LC CDR2 comprising the sequence of SEQ ID NO: 3653, and a LC CDR3 comprising the sequence of SEQ ID NO: 8; or
(ii) a VH comprising a HC CDR1 comprising the sequence of SEQ ID NO: 542, a HC CDR2 comprising the sequence of SEQ ID NO: 543, and a HC CDR3 comprising the sequence of SEQ ID NO: 544, and a VL comprising a LC CDR1 comprising the sequence of SEQ ID NO: 545, a LC CDR2 comprising the sequence of SEQ ID NO: 546, and a LC CDR3 comprising the sequence of SEQ ID NO: 547.

156. The multispecific molecule of claim 143, wherein the TCRβV-binding moiety comprises:
(i) a VH comprising a sequence having at least 70% sequence identity to the sequence of SEQ ID NO: 1346 and a VL comprising a sequence having at least 70% sequence identity to the sequence of SEQ ID NO: 1349,
wherein the VH comprises a HC CDR1 comprising the sequence of SEQ ID NO: 3650, a HC CDR2 comprising the sequence of SEQ ID NO: 3651, and a HC CDR3 comprising the sequence of SEQ ID NO: 5, and the VL comprises a LC CDR1 comprising the sequence of SEQ ID NO: 3655, a LC CDR2 comprising the sequence of SEQ ID NO: 3653, and a LC CDR3 comprising the sequence of SEQ ID NO: 8; or
(ii) a VH comprising a sequence having at least 70% sequence identity to the sequence of SEQ ID NO: 541 and a VL comprising a sequence having at least 70% sequence identity the sequence of SEQ ID NO: 3527,
wherein the VH comprises a HC CDR1 comprising the sequence of SEQ ID NO: 542, a HC CDR2 comprising the sequence of SEQ ID NO: 543, and a HC CDR3 comprising the sequence of SEQ ID NO: 544, and the VL comprises a LC CDR1 comprising the sequence of SEQ ID NO: 545, a LC CDR2 comprising the sequence of SEQ ID NO: 546, and a LC CDR3 comprising the sequence of SEQ ID NO: 547.

157. The multispecific molecule of claim 143, wherein the TCRβV-binding moiety comprises:
(i) a VH comprising the sequence of SEQ ID NO: 1346 and a VL comprising the sequence of SEQ ID NO: 1349; or
(ii) a VH comprising the sequence of SEQ ID NO: 541 and a VL comprising the sequence of SEQ ID NO: 3527.

158. The multispecific molecule of claim 143, wherein the TCRβV-binding moiety comprises an scFv comprising:
(i) the sequence of SEQ ID NO: 1346 operatively linked to the sequence of SEQ ID NO: 1349; or
(ii) the sequence of SEQ ID NO: 541 operatively linked to the sequence of SEQ ID NO: 3527.

159. The multispecific molecule of claim 143, wherein the TCRβV-binding moiety comprises an scFv comprising:
(i) the sequence of SEQ ID NO: 1346 operatively linked to the sequence of SEQ ID NO: 1349 via a linker comprising the sequence of SEQ ID NO: 3801; or
(ii) the sequence of SEQ ID NO: 541 operatively linked to the sequence of SEQ ID NO: 3527 via a linker comprising the sequence of SEQ ID NO: 3801.

160. The multispecific molecule of claim 143, wherein the TCRβV-binding moiety comprises:
(i) an scFv comprising a sequence having at least 70% sequence identity to the sequence of SEQ ID NO: 1331,
wherein a VH of the scFv comprises a HC CDR1 comprising the sequence of SEQ ID NO: 3650, a HC CDR2 comprising the sequence of SEQ ID NO: 3651, and a HC CDR3 comprising the sequence of SEQ ID NO: 5, and a VL of the scFv comprises a LC CDR1 comprising the sequence of SEQ ID NO: 3655, a LC CDR2 comprising the sequence of SEQ ID NO: 3653, and a LC CDR3 comprising the sequence of SEQ ID NO: 8; or
(ii) an scFv comprising a sequence having at least 70% sequence identity to the sequence of SEQ ID NO: 1376,
wherein a VH of the scFv comprises a HC CDR1 comprising the sequence of SEQ ID NO: 542, a HC CDR2 comprising the sequence of SEQ ID NO: 543, and a HC CDR3 comprising the sequence of SEQ ID NO: 544, and a VL of the scFv comprises a LC CDR1 comprising the sequence of SEQ ID NO: 545, a LC CDR2 comprising the sequence of SEQ ID NO: 546, and a LC CDR3 comprising the sequence of SEQ ID NO: 547.

161. The multispecific molecule of claim 143, wherein the TCRβV-binding moiety comprises an scFv comprising the sequence of SEQ ID NO: 1331 or an scFv comprising the sequence of SEQ ID NO: 1376.

162. The multispecific molecule of claim 143, wherein the at least one cytokine molecule or a functional fragment or variant thereof comprises IL-2 or a functional fragment or variant thereof comprising a sequence having at least 90% sequence identity to the sequence of SEQ ID NO: 2270 or the sequence of SEQ ID NO: 2191.

163. The multispecific molecule of claim 144, wherein the at least one cytokine molecule or a functional fragment or variant thereof comprises IL-15 or a functional fragment or variant thereof comprising a sequence having at least 90% sequence identity to the sequence of SEQ ID NO: 2170 or the sequence of SEQ ID NO: 3799; (iv) IL-7 or a functional fragment or variant thereof comprising a sequence having at least 75% sequence identity to the sequence of SEQ ID NO: 3540; or (v) IL-21 or a functional fragment or variant thereof comprising a sequence having at least 75% sequence identity to the sequence of SEQ ID NO: 2193.

164. The multispecific molecule of claim 163, wherein the at least one cytokine molecule or a functional fragment or variant thereof further comprises the sequence of SEQ ID NO: 3523 linked to the sequence having at least 90% sequence identity to the sequence of SEQ ID NO: 2170 or to the sequence having at least 90% sequence identity to the he sequence of SEQ ID NO: 3799.

165. The multispecific molecule of claim 143, wherein the at least one cytokine molecule or a functional fragment or variant thereof comprises IL-12 or a functional fragment or variant thereof comprising a sequence having at least 75% sequence identity to the sequence of SEQ ID NO: 3542.

166. The multispecific molecule of claim 143, wherein the at least one cytokine molecule or a functional fragment or variant thereof comprises IL-7 or a functional fragment or variant thereof comprising a sequence having at least 90% sequence identity to the sequence of SEQ ID NO: 3540.

167. The multispecific molecule of claim 143, wherein the at least one cytokine molecule or a functional fragment or variant thereof comprises IL-21 or a functional fragment or variant thereof comprising a sequence having at least 90% sequence identity to the sequence of SEQ ID NO: 2193.

168. The multispecific molecule of claim 164, wherein the Fc domain of the first polypeptide chain and the Fc domain of the second polypeptide chain is selected from the group consisting of a human IgG1 Fc region or a fragment thereof, a human IgG2 Fc region or a fragment thereof, and a human IgG4 Fc region or a fragment thereof.

169. The multispecific molecule of claim 164, wherein the Fc domain of the first polypeptide chain and the Fc domain of the second polypeptide chain comprises:
(i) an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface; and/or
(ii) an Asn297Ala (N297A) mutation or a Leu234Ala/Leu235Ala (LALA) mutation.

170. The multispecific molecule of claim 164, wherein
(i) the Fc domain of the first polypeptide chain comprises a sequence having at least 90% sequence identity to the sequence of SEQ ID NO: 3649 or SEQ ID NO: 3792, and the Fc domain of the second polypeptide chain comprises a sequence having at least 90% sequence identity to the sequence of SEQ ID NO: 3648 or SEQ ID NO: 3794, or
(ii) the Fc domain of the first polypeptide chain comprises a sequence having at least 90% sequence identity to the sequence of SEQ ID NO: 3648 or SEQ ID NO: 3794, and the Fc domain of the second polypeptide chain comprises a sequence having at least 90% sequence identity to the sequence of SEQ ID NO: 3649 or SEQ ID NO: 3792.

171. The multispecific molecule of claim 164, wherein
(i) the Fc domain of the first polypeptide chain comprises a sequence having the sequence of SEQ ID NO: 3649 or SEQ ID NO: 3792, and the Fc domain of the second polypeptide chain comprises the sequence of SEQ ID NO: 3648 or SEQ ID NO: 3794, or
(ii) the Fc domain of the first polypeptide chain comprises the sequence of SEQ ID NO: 3648 or SEQ ID NO: 3794, and the Fc domain of the second polypeptide chain comprises the sequence of SEQ ID NO: 3649 or SEQ ID NO: 3792.

172. The multispecific molecule of claim 143, wherein the multispecific molecule consists of a first polypeptide chain, a second polypeptide chain, and a third polypeptide chain, wherein:
(A)(i) the first polypeptide chain comprises the sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO: 2270 operatively linked to the sequence of SEQ ID NO: 3648; and
(iii) the third polypeptide chain comprises the sequence of SEQ ID NO: 527 operatively linked to the sequence of SEQ ID NO: 3644;
(B)(i) the first polypeptide chain comprises a first sequence comprising the sequence of SEQ ID NO: 523 operatively linked to a second sequence comprising the sequence of SEQ ID NO: 523 operatively linked to the sequence of SEQ ID NO: 3649;
(ii) the second polypeptide chain comprises the sequence of SEQ ID NO: 1331 operatively linked to the sequence of SEQ ID NO: 2270 operatively linked to the sequence of SEQ ID NO: 3648; and
(iii) the third polypeptide chain comprises the sequence of SEQ ID NO: 527 operatively linked to the sequence of SEQ ID NO: 3644;
(C)(i) the first polypeptide chain comprises the sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558;
(D)(i) the first polypeptide chain comprises a first sequence comprising the sequence of SEQ ID NO: 537 operatively linked to a second sequence comprising the sequence of SEQ ID NO: 537 operatively linked to the sequence of SEQ ID NO: 559;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 561 operatively linked to the sequence of SEQ ID NO: 558; or
(E)(i) the first polypeptide chain comprises the sequence of SEQ ID NO: 580 operatively linked to the sequence of SEQ ID NO: 584;
(ii) the second polypeptide chain comprises the sequence of SEQ ID NO: 1376 operatively linked to the sequence of SEQ ID NO: 2191 operatively linked to the sequence of SEQ ID NO: 3533; and
(iii) the third polypeptide chain comprises the sequence of SEQ ID NO: 585 operatively linked to the sequence of SEQ ID NO: 3528.

173. The multispecific molecule of claim 150, wherein the multispecific molecule consists of a first polypeptide chain, a second polypeptide chain, and a third polypeptide chain, wherein:
(A)(i) the first polypeptide chain comprises a sequence having at least 70% sequence identity to the sequence of SEQ ID NO: 501;
(ii) the second polypeptide chain comprises a sequence having at least 70% sequence identity to the sequence of SEQ ID NO: 3800; and
(iii) the third polypeptide chain comprises a sequence having at least 70% sequence identity to the sequence of SEQ ID NO: 502;
(B)(i) the first polypeptide chain comprises a sequence having at least 70% sequence identity to the sequence of SEQ ID NO: 590;
(ii) the second polypeptide chain comprises a sequence having at least 70% sequence identity to the sequence of SEQ ID NO: 3800; and
(iii) the third polypeptide chain comprises a sequence having at least 70% sequence identity to the sequence of SEQ ID NO: 502;
(C)(i) the first polypeptide chain comprises a sequence having at least 70% sequence identity to the sequence of SEQ ID NO: 509;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 510;
(D)(i) the first polypeptide chain comprises a sequence having at least 70% sequence identity to the sequence of SEQ ID NO: 512;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 510; or
(E)(i) the first polypeptide chain comprises a sequence having at least 70% sequence identity to the sequence of SEQ ID NO: 519;
(ii) the second polypeptide chain comprises a sequence having at least 70% sequence identity to the sequence of SEQ ID NO: 511; and
(iii) the third polypeptide chain comprises a sequence having at least 70% sequence identity to the sequence of SEQ ID NO: 520.

174. The multispecific molecule of claim 143, wherein the multispecific molecule consists of a first polypeptide chain, a second polypeptide chain, and a third polypeptide chain, wherein:
(A)(i) the first polypeptide chain comprises the sequence of SEQ ID NO: 501;
(ii) the second polypeptide chain comprises the sequence of SEQ ID NO: 3800; and
(iii) the third polypeptide chain comprises the sequence of SEQ ID NO: 502;
(B)(i) the first polypeptide chain comprises the sequence of SEQ ID NO: 590;
(ii) the second polypeptide chain comprises the sequence of SEQ ID NO: 3800; and
(iii) the third polypeptide chain comprises the sequence of SEQ ID NO: 502;
(C)(i) the first polypeptide chain comprises the sequence of SEQ ID NO: 509;
(ii) the second polypeptide chain comprises the sequence of SEQ ID NO: 511; and
(iii) the third polypeptide chain comprises the sequence of SEQ ID NO: 510;
(D)(i) the first polypeptide chain comprises the sequence of SEQ ID NO: 512;
(ii) the second polypeptide chain comprises the sequence of SEQ ID NO: 511; and
(iii) the third polypeptide chain comprises the sequence of SEQ ID NO: 510; or
(E)(i) the first polypeptide chain comprises the sequence of SEQ ID NO: 519;
(ii) the second polypeptide chain comprises the sequence of SEQ ID NO: 511; and
(iii) the third polypeptide chain comprises the sequence of SEQ ID NO: 520.

175. A pharmaceutical composition comprising the multispecific molecule of claim 143, and a pharmaceutically acceptable carrier, excipient, or diluent.

176. A method of treating a condition or disease in a subject in need thereof comprising administering to the subject a therapeutically effective amount of the multispecific molecule of claim 143, thereby treating the condition or disease in the subject, wherein the condition or disease is cancer.

177. The method of claim 176, wherein the cancer is a solid tumor, a hematological cancer, a metastatic cancer, a soft tissue tumor, or any combination thereof.

178. The method of claim 176, wherein:
(i) the cancer is a solid tumor selected from the group consisting of melanoma, pancreatic cancer, breast cancer, colorectal cancer, lung cancer, skin cancer, ovarian cancer, liver cancer, and any combination thereof; or
(ii) the cancer is a hematological cancer selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and any combination thereof.

179. An antibody molecule or an antigen binding domain comprises:
(i) a VH comprising a HC CDR1 comprising the sequence of SEQ ID NO: 524, a HC CDR2 comprising the sequence of SEQ ID NO: 525, and a HC CDR3 comprising the sequence of SEQ ID NO: 526, and a VL comprising a LC CDR1 comprising the sequence of SEQ ID NO: 1168, a LC CDR2 comprising the sequence of SEQ ID NO: 1295, and a LC CDR3 comprising the sequence of SEQ ID NO: 528;
(ii) a VH comprising a HC CDR1 comprising the sequence of SEQ ID NO: 538, a HC CDR2 comprising the sequence of SEQ ID NO: 539, and a HC CDR3 comprising the sequence of SEQ ID NO: 540, and a VL comprising a LC CDR1 comprising the sequence of SEQ ID NO: 562, a LC CDR2 comprising the sequence of SEQ ID NO: 563, and a LC CDR3 comprising the sequence of SEQ ID NO: 564; or
(iii) a VH comprising a HC CDR1 comprising the sequence of SEQ ID NO: 581, a HC CDR2 comprising the sequence of SEQ ID NO: 582, and a HC CDR3 comprising the sequence of SEQ ID NO: 583, and a VL comprising a LC CDR1 comprising the sequence of SEQ ID NO: 586, a LC CDR2 comprising the sequence of SEQ ID NO: 587, and a LC CDR3 comprising the sequence of SEQ ID NO: 588.
